(19) United States
(12) Patent Application Publication
(10) Pub. No.: US 2025/0171462 A1
(43) Pub. Date: May 29, 2025

(54) COMPOUNDS, COMPOSITIONS, AND METHODS

(71) Applicant: NICO Therapeutics, Inc., Chicago, IL (US)

(72) Inventors: Lydia A. Auch, San francisco, CA (US); Alex L. Bagdasarian, South San Francisco, CA (US); Cyril Bucher, San Mateo, CA (US); Robert A. Craig, II, South San Francisco, CA (US); Javier de Vicente Fidalgo, Foster City, CA (US); Anthony A. Estrada, Danville, CA (US); Brian M. Fox, Redwood City, CA (US); Benjamin J. Huffman, Brisbane, CA (US); Katrina W. Lexa, San Francisco, CA (US); Takashi Miyamoto, Burlingame, CA (US); Maksim Osipov, Redwood City, CA (US); Arun Thottumkara, San Francisco, CA (US)

(21) Appl. No.: 18/842,014

(22) PCT Filed: Mar. 1, 2023
(86) PCT No.: PCT/US2023/063523

§ 371 Date: Aug. 27, 2024

Related U.S. Application Data
(60) Provisional application No. 63/345,869, filed on May 25, 2022.
(60) Provisional application No. 63/315,944, filed on Mar. 2, 2022.

(51) Int. Cl. C07D 498/04; A61K 31/4439; A61K 31/444; A61K 31/497; A61K 31/4995; A61K 31/501; A61K 31/506; A61K 31/53; A61K 31/5377; A61K 31/553; C07D 213/40; C07D 237/08; C07D 239/26; C07D 401/14; C07D 403/12; C07D 403/14; C07D 413/14; C07D 471/08; C07D 487/08

(52) U.S. Cl. CPC C07D 498/04; A61K 31/4439; A61K 31/444; A61K 31/497; A61K 31/4995; A61K 31/501; A61K 31/506; A61K 31/53; A61K 31/5377; A61K 31/553; C07D 213/40; C07D 237/08; C07D 239/26; C07D 401/14; C07D 403/12; C07D 403/14; C07D 413/14; C07D 471/08; C07D 487/08

(57) ABSTRACT

The present disclosure relates generally to small molecule inhibitors of Sterile Alpha and TIR Motif containing 1 (SARM1) protein, or a pharmaceutically acceptable salt, isotopically enriched analog, stereoisomer, mixture of stereoisomers, or prodrug thereof, methods of making and intermediates thereof, and methods of using thereof.

### CROSS-REFERENCE TO RELATED APPLICATIONS

This application claims the benefit under 35 U.S.C. § 119(e) of U.S. Provisional Application Ser. No. 63/315,944, filed Mar. 2, 2022, and U.S. Provisional Application Ser. No. 63/345,869, filed May 25, 2022 the contents of which are hereby incorporated by reference in their entirety.

### FIELD

The present disclosure relates generally to small molecule modulators of Sterile Alpha and TIR Motif containing 1 (SARM1) protein, and their use as therapeutic agents.

### BACKGROUND

Neurodegenerative diseases are a class of progressive neurological disorders, in which nerve cells malfunction and ultimately die. The degradation of neurons in those suffering from a neurodegenerative disease can present as a wide variety of symptoms, including changes in mood and behavior, agitation, sensory disturbances, motor and cognitive difficulties, and memory loss, which can progress to inability to move or speak, dementia, and ultimately death.

Axonal degeneration has been identified as an important pathology in most neurodegenerative diseases. Axons are vulnerable to both mechanical injury (Wallerian degeneration) and disease (Wallerian-like degeneration).

In healthy axons, SARM1's N-terminus interacts with the TIR domain, preventing TIR dimerization and subsequent enzymatic cleavage of NAD⁺. However, under neuronal injury or disease conditions, SARM1's N-terminus-TIR domain interaction is disrupted, allowing TIR multimerization to occur, followed by a rapid loss of NAD⁺ and associated axon degeneration.

### DESCRIPTION

Provided herein are compounds, or a pharmaceutically acceptable salt, isotopically enriched analog, stereoisomer, mixture of stereoisomers, or prodrug thereof, that are useful in treating and/or preventing diseases mediated, at least in part, by SARM1.

In certain embodiments, provided are compounds that inhibit SARM1.

In another embodiment, provided is a pharmaceutical composition comprising a compound as described herein, or a pharmaceutically acceptable salt, isotopically enriched analog, stereoisomer, mixture of stereoisomers, or prodrug thereof, and a pharmaceutically acceptable carrier.

In another embodiment, provided is a method for treating a disease or condition mediated, at least in part, by SARM1, the method comprising administering to a subject in need thereof a therapeutically effective amount of a compound disclosed herein, or a pharmaceutically acceptable salt, isotopically enriched analog, stereoisomer, mixture of stereoisomers, or prodrug thereof.

The disclosure also provides compositions, including pharmaceutical compositions, kits that include the compounds, or a pharmaceutically acceptable salt, isotopically enriched analog, stereoisomer, mixture of stereoisomers, or prodrug thereof, methods of using (or administering) and making the compounds, or a pharmaceutically acceptable salt, isotopically enriched analog, stereoisomer, mixture of stereoisomers, or prodrug thereof, and intermediates thereof.

The disclosure further provides compounds, or a pharmaceutically acceptable salt, isotopically enriched analog, stereoisomer, mixture of stereoisomers, or prodrug thereof, or compositions thereof for use in a method of treating a disease, disorder, or condition that is mediated, at least in part, by SARM1.

Moreover, the disclosure provides uses of the compounds, or a pharmaceutically acceptable salt, isotopically enriched analog, stereoisomer, mixture of stereoisomers, or prodrug thereof, or compositions thereof in the manufacture of a medicament for the treatment of a disease, disorder, or condition that is mediated, at least in part, by SARM1.

The description herein sets forth exemplary embodiments of the present technology. It should be recognized, however, that such description is not intended as a limitation on the scope of the present disclosure but is instead provided as a description of exemplary embodiments.

### 1. Definitions

A dash (“-”) that is not between two letters or symbols is used to indicate a point of attachment for a substituent. For example, —C(O)NH₂ is attached through the carbon atom. A dash at the front or end of a chemical group is a matter of convenience; chemical groups may be depicted with or without one or more dashes without losing their ordinary meaning. A wavy line or a dashed line drawn through a line in a structure indicates a specified point of attachment of a group. Unless chemically or structurally required, no directionality or stereochemistry is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount ±10%. In other embodiments, the term “about” includes the indicated amount ±5%. In certain other embodiments, the term “about” includes the indicated amount ±1%. Also, to the term “about X” includes description of “X”. Also, the singular forms “a” and “the” include plural references unless the context clearly dictates otherwise. Thus, e.g., reference to “the compound” includes a plurality of such compounds and reference to “the assay” includes reference to one or more assays and equivalents thereof known to those skilled in the art.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl) or 1 to 4 carbon atoms (i.e., C₁₋₄ alkyl). Examples of alkyl groups include, e.g., methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂), and tert-butyl (i.e., —C(CH₃)₃); and “propyl” includes n-propyl (i.e., —(CH₂)₂CH₃) and isopropyl (i.e., —CH(CH₃)₂).

Certain commonly used alternative chemical names may be used. For example, a divalent group such as a divalent “alkyl” group, a divalent “aryl” group, a divalent heteroaryl group, etc., may also be referred to as an “alkylene” group or an “alkylenyl” group (for example, methylenyl, ethylenyl, and propylenyl), an “arylene” group or an “arylenyl” group (for example, phenylenyl or napthylenyl, or quinolinyl for heteroarylene), respectively. Also, unless indicated explicitly otherwise, where combinations of groups are referred to herein as one moiety, e.g., arylalkyl or aralkyl, the last mentioned group contains the atom by which the moiety is attached to the rest of the molecule.

“Alkenyl” refers to an alkyl group containing at least one (e.g., 1-3, or 1) carbon-carbon double bond and having from 2 to 20 carbon atoms (i.e., C₂₋₂₀ alkenyl), 2 to 12 carbon atoms (i.e., C₂₋₁₂ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include, e.g., ethenyl, propenyl, butadienyl (including 1,2-butadienyl and 1,3-butadienyl).

“Alkynyl” refers to an alkyl group containing at least one (e.g., 1-3, or 1) carbon-carbon triple bond and having from 2 to 20 carbon atoms (i.e., C₂₋₂₀ alkynyl), 2 to 12 carbon atoms (i.e., C₂₋₁₂ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O—”. Examples of alkoxy groups include, e.g., methoxy, ethoxy, n-propoxy, iso-propoxy, n-butoxy, tert-butoxy, sec-butoxy, n-pentoxy, n-hexoxy, and 1,2-dimethylbutoxy.

“Alkoxyalkyl” refers to the group “alkyl-O-alkyl”.

“Alkylthio” refers to the group “alkyl-S—”. “Alkylsulfinyl” refers to the group “alkyl-S(O)—”. “Alkylsulfonyl” refers to the group “alkyl-S(O)₂—”. “Alkylsulfonylalkyl” refers to -alkyl-S(O)₂-alkyl.

“Acyl” refers to a group —C(O)R^{y}, wherein R^{y} is hydrogen, alkyl, alkenyl, alkynyl, cycloalkyl, heterocyclyl, aryl, heteroalkyl, or heteroaryl; each of which may be optionally substituted, as defined herein. Examples of acyl include, e.g., formyl, acetyl, cyclohexylcarbonyl, cyclohexylmethyl-carbonyl, and benzoyl.

“Amido” refers to both a “C-amido” group which refers to the group —C(O)NR^{y}R^{z} and an “N-amido” group which refers to the group —NR^{y}C(O)R^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein, or R^{y} and R^{z} are taken together to form a cycloalkyl or heterocyclyl; each of which may be optionally substituted, as defined herein.

“Amino” refers to the group —NR^{y}R^{z} wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Amidino” refers to —C(NR^{y})(NR^{z}₂), wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, e.g., phenyl, naphthyl, fluorenyl, and anthryl. Aryl, however, does not encompass or overlap in any way with heteroaryl defined below. If one or more aryl groups are fused with a heteroaryl, the resulting ring system is heteroaryl regardless of point of attachment. If one or more aryl groups are fused with a heterocyclyl, the resulting ring system is heterocyclyl regardless of point of attachment. If one or more aryl groups are fused with a cycloalkyl, the resulting ring system is cycloalkyl regardless of point of attachment.

“Arylalkyl” or “Aralkyl” refers to the group “aryl-alkyl-”.

“Carbamoyl” refers to both an “O-carbamoyl” group which refers to the group —O—C(O)NR^{y}R^{z} and an “N-carbamoyl” group which refers to the group —NR^{y}C(O)OR^{z}, wherein R^{y} and R^{z} are independently hydrogen, alkyl, alkenyl, alkynyl, cycloalkyl, heterocyclyl, aryl, heteroalkyl, or heteroaryl; each of which may be optionally substituted, as defined herein.

“Carboxyl ester” or “ester” refer to both —OC(O)R^{x} and —C(O)OR^{x}, wherein R^{x} is alkyl, alkenyl, alkynyl, cycloalkyl, heterocyclyl, aryl, heteroalkyl, or heteroaryl; each of which may be optionally substituted, as defined herein.

“Cyanoalkyl” refers to refers to an alkyl group as defined above, wherein one or more (e.g., 1 or 2) hydrogen atoms are replaced by a cyano (—CN) group.

“Cycloalkyl” refers to a saturated or partially unsaturated cyclic alkyl group having a single ring or multiple rings including fused, bridged, and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl), 3 to 14 ring carbon atoms (i.e., C₃₋₁₂ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Monocyclic groups include, for example, cyclopropyl, cyclobutyl, cyclopentyl, cyclohexyl, cycloheptyl, and cyclooctyl. Polycyclic groups include, for example, bicyclo[2.2.1]heptanyl, bicyclo[2.2.2]octanyl, adamantyl, norbornyl, decalinyl, 7,7-dimethyl-bicyclo[2.2.1]heptanyl, and the like.

Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom, for example spiro[2.5]octanyl, spiro[4.5]decanyl, or spiro[5.5]undecanyl.

“Cycloalkylalkyl” refers to the group “cycloalkyl-alkyl-”.

“Imino” refers to a group —C(NR^{y})R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

“Imido” refers to a group —C(O)NR^{y}C(O)R^{z}, wherein R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

“Halogen” or “halo” refers to atoms occupying group VIIA of the periodic table, such as fluoro, chloro, bromo, or iodo.

“Haloalkyl” refers to an unbranched or branched alkyl group as defined above, wherein one or more (e.g., 1 to 6 or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Haloalkoxy” refers to an alkoxy group as defined above, wherein one or more (e.g., 1 to 6 or 1 to 3) hydrogen atoms are replaced by a halogen.

“Haloalkoxyalkyl” refers to an alkoxyalkyl group as defined above, wherein one or more (e.g., 1 to 6 or 1 to 3) hydrogen atoms are replaced by a halogen.

“Hydroxyalkyl” refers to an alkyl group as defined above, wherein one or more (e.g., 1 to 6 or 1 to 3) hydrogen atoms are replaced by a hydroxy group.

“Heteroalkyl” refers to an alkyl group in which one or more of the carbon atoms (and any associated hydrogen atoms), excluding any terminal carbon atom(s), are each independently replaced with the same or different heteroatomic group, provided the point of attachment to the remainder of the molecule is through a carbon atom. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR^{y}—, —O—, —S—, —S(O)—, —S(O)₂—, and the like, wherein R is hydrogen, alkyl, alkenyl, alkynyl, cycloalkyl, heterocyclyl, aryl, heteroalkyl, or heteroaryl; each of which may be optionally substituted, as defined herein. Examples of heteroalkyl groups include, e.g., ethers (e.g., —CH₂OCH₃, —CH(CH₃)OCH₃, —CH₂CH₂OCH₃, —CH₂CH₂OCH₂CH₂OCH₃, etc.), thioethers (e.g., —CH₂SCH₃, —CH(CH₃)SCH₃, —CH₂CH₂SCH₃, —CH₂CH₂SCH₂CH₂SCH₃, etc.), sulfones (e.g., —CH₂S(O)₂CH₃, —CH(CH₃)S(O)₂CH₃, —CH₂CH₂S(O)₂CH₃, —CH₂CH₂S(O)₂CH₂CH₂OCH₃, etc.), and amines (e.g., —CH₂NR^{y}CH₃, —CH(CH₃)NR^{y}CH₃, —CH₂CH₂NR^{y}CH₃, —CH₂CH₂NR^{y}CH₂CH₂NR^{y}CH₃, etc., where R^{y} is hydrogen, alkyl, alkenyl, alkynyl, cycloalkyl, heterocyclyl, aryl, heteroalkyl, or heteroaryl; each of which may be optionally substituted, as defined herein). As used herein, heteroalkyl includes 2 to 10 carbon atoms, 2 to 8 carbon atoms, or 2 to 4 carbon atoms; and 1 to 3 heteroatoms, 1 to 2 heteroatoms, or 1 heteroatom.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen, and sulfur. In certain instances, heteroaryl includes 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen, and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl, and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl, and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heteroarylalkyl” refers to the group “heteroaryl-alkyl-”.

“Heterocyclyl” refers to a saturated or partially unsaturated cyclic alkyl group, with one or more ring heteroatoms independently selected from nitrogen, oxygen, and sulfur. The term “heterocyclyl” includes heterocycloalkenyl groups (i.e., the heterocyclyl group having at least one double bond), bridged-heterocyclyl groups, fused-heterocyclyl groups, and spiro-heterocyclyl groups. A heterocyclyl may be a single ring or multiple rings wherein the multiple rings may be fused, bridged, or spiro, and may comprise one or more (e.g., 1 to 3) oxo (═O) or N-oxide (—O—) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to a cycloalkyl, an aryl, or heteroaryl ring, regardless of the attachment to the remainder of the molecule. As used herein, heterocyclyl has 2 to 20 ring carbon atoms (i.e., C₂₋₂₀ heterocyclyl), 2 to 12 ring carbon atoms (i.e., C₂12 heterocyclyl), 2 to 10 ring carbon atoms (i.e., C₂₋₂₀ heterocyclyl), 2 to 8 ring carbon atoms (i.e., C₂₋₈ heterocyclyl), 3 to 12 ring carbon atoms (i.e., C₃₋₁₂ heterocyclyl), 3 to 8 ring carbon atoms (i.e., C₃_8 heterocyclyl), or 3 to 6 ring carbon atoms (i.e., C₃₋₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur, or oxygen. Examples of heterocyclyl groups include, e.g., azetidinyl, azepinyl, benzodioxolyl, benzo[b][1,4]dioxepinyl, 1,4-benzodioxanyl, benzopyranyl, benzodioxinyl, benzopyranonyl, benzofuranonyl, dioxolanyl, dihydropyranyl, hydropyranyl, thienyl[1,3]dithianyl, decahydroisoquinolyl, furanonyl, imidazolinyl, imidazolidinyl, indolinyl, indolizinyl, isoindolinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, oxiranyl, oxetanyl, phenothiazinyl, phenoxazinyl, piperidinyl, piperazinyl, 4-piperidonyl, pyrrolidinyl, pyrazolidinyl, quinuclidinyl, thiazolidinyl, tetrahydrofuryl, tetrahydropyranyl, trithianyl, tetrahydroquinolinyl, thiophenyl (i.e., thienyl), thiomorpholinyl, thiamorpholinyl, 1-oxo-thiomorpholinyl, and 1,1-dioxo-thiomorpholinyl. The term “heterocyclyl” also includes “spiroheterocyclyl” when there are two positions for substitution on the same carbon atom. Examples of the spiro-heterocyclyl rings include, e.g., bicyclic and tricyclic ring systems, such as oxabicyclo[2.2.2]octanyl, 2-oxa-7-azaspiro[3.5]nonanyl, 2-oxa-6-azaspiro[3.4]octanyl, and 6-oxa-1-azaspiro[3.3]heptanyl. Examples of the fused-heterocyclyl rings include, but are not limited to, 1,2,3,4-tetrahydroisoquinolinyl, 4,5,6,7-tetrahydrothieno[2,3-c]pyridinyl, indolinyl, and isoindolinyl, where the heterocyclyl can be bound via either ring of the fused system.

“Heterocyclylalkyl” refers to the group “heterocyclyl-alkyl-.”

“Oxime” refers to the group —CR^{y}(═NOH) wherein R^{y} is hydrogen, alkyl, alkenyl, alkynyl, cycloalkyl, heterocyclyl, aryl, heteroalkyl, or heteroaryl; each of which may be optionally substituted, as defined herein.

“Sulfonyl” refers to the group —S(O)₂R^{y}, where R^{y} is hydrogen, alkyl, alkenyl, alkynyl, cycloalkyl, heterocyclyl, aryl, heteroalkyl, or heteroaryl; each of which may be optionally substituted, as defined herein. Examples of sulfonyl are methylsulfonyl, ethylsulfonyl, phenylsulfonyl, and toluenesulfonyl.

“Sulfinyl” refers to the group —S(O)R^{y}, where R^{y} is hydrogen, alkyl, alkenyl, alkynyl, cycloalkyl, heterocyclyl, aryl, heteroalkyl, or heteroaryl; each of which may be optionally substituted, as defined herein. Examples of sulfinyl are methylsulfinyl, ethylsulfinyl, phenylsulfinyl, and toluenesulfinyl.

“Sulfonamido” refers to the groups —SO₂NR^{y}R^{z} and —NR^{y}SO₂R^{z}, where R^{y} and R^{z} are each independently hydrogen, alkyl, alkenyl, alkynyl, cycloalkyl, heterocyclyl, aryl, heteroalkyl, or heteroaryl; each of which may be optionally substituted, as defined herein.

The terms “optional” or “optionally” means that the subsequently described event or circumstance may or may not occur and that the description includes instances where said event or circumstance occurs and instances in which it does not. Also, the term “optionally substituted” refers to any one or more (e.g., 1 to 5 or 1 to 3) hydrogen atoms on the designated atom or group may or may not be replaced by a moiety other than hydrogen.

The term “substituted” used herein means any of the above groups (i.e., alkyl, alkenyl, alkynyl, alkylene, alkoxy, haloalkyl, haloalkoxy, cycloalkyl, aryl, heterocyclyl, heteroaryl, and/or heteroalkyl) wherein at least one (e.g., 1 to 5 or 1 to 3) hydrogen atom is replaced by a bond to a non-hydrogen atom such as, but not limited to alkyl, alkenyl, alkynyl, alkoxy, alkylthio, acyl, amido, amino, amidino, aryl, aralkyl, azido, carbamoyl, carboxyl, carboxyl ester, cyano, cycloalkyl, cycloalkylalkyl, guanadino, halo, haloalkyl, haloalkoxy, hydroxyalkyl, heteroalkyl, heteroaryl, heteroarylalkyl, heterocyclyl, heterocyclylalkyl, —NHNH₂, =NNH₂, imino, imido, hydroxy, oxo, oxime, nitro, sulfonyl, sulfinyl, alkylsulfonyl, alkylsulfinyl, thiocyanate, —S(O)OH, —S(O)₂OH, sulfonamido, thiol, thioxo, N-oxide, or —Si(R^{y})₃, wherein each R^{y} is independently hydrogen, alkyl, alkenyl, alkynyl, heteroalkyl, cycloalkyl, aryl, heteroaryl, or heterocyclyl.

In certain embodiments, “substituted” includes any of the above alkyl, alkenyl, alkynyl, cycloalkyl, heterocyclyl, aryl, or heteroaryl groups in which one or more (e.g., 1 to 5 or 1 to 3) hydrogen atoms are independently replaced with deuterium, halo, cyano, nitro, azido, oxo, alkyl, alkenyl, alkynyl, haloalkyl, cycloalkyl, heterocyclyl, aryl, heteroaryl, —NR^{g}R^{h}, —NR^{g}C(O)R^{h}, —NR^{g}C(O)NR^{g}R^{h}, —NR^{g}C(O)OR^{h}, —NR^{g}S(O)₁₋₂R^{h}, —C(O)R^{g}, —C(O)OR^{g}, —OC(O)OR^{g}, —OC(O)R^{g}, —C(O)NR^{g}R^{h}, —OC(O)NR^{g}R^{h}, —OR^{g}, —SR^{g}, —S(O)R^{g}, —S(O)₂R^{g}, —OS(O)₁₋₂R^{g}, —S(O)₁₋₂OR^{g}, —NR^{g}S(O)₁₋₂NR^{g}R^{h}, ═NSO₂R^{g}, ═NOR^{g}, —S(O)₁₋₂NR^{g}R^{h}, —SF₅, —SCF₃, or —OCF₃. In certain embodiments, “substituted” also means any of the above groups in which one or more (e.g., 1 to 5 or 1 to 3) hydrogen atoms are replaced with —C(O)R^{g}, —C(O)OR^{g}, —C(O)NR^{g}R^{h}, —CH₂SO₂R^{g}, or —CH₂SO₂NR^{g}R^{h}. In the foregoing, R^{g} and R^{h} are the same or different and independently hydrogen, alkyl, alkenyl, alkynyl, alkoxy, thioalkyl, aryl, aralkyl, cycloalkyl, cycloalkylalkyl, haloalkyl, heterocyclyl, heterocyclylalkyl, heteroaryl, and/or heteroarylalkyl. In certain embodiments, “substituted” also means any of the above groups in which one or more (e.g., 1 to 5 or 1 to 3) hydrogen atoms are replaced by a bond to an amino, cyano, hydroxy, imino, nitro, oxo, thioxo, halo, alkyl, alkoxy, alkylamino, thioalkyl, aryl, aralkyl, cycloalkyl, cycloalkylalkyl, haloalkyl, heterocyclyl, N-heterocyclyl, heterocyclylalkyl, heteroaryl, and/or heteroarylalkyl, or two of R^{g} and R^{h} and Rⁱ are taken together with the atoms to which they are attached to form a heterocyclyl ring optionally substituted with oxo, halo, or alkyl optionally substituted with oxo, halo, amino, hydroxy, or alkoxy.

Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein.

In certain embodiments, as used herein, the phrase “one or more” refers to one to five. In certain embodiments, as used herein, the phrase “one or more” refers to one to three.

Any compound or structure given herein, is also intended to represent unlabeled forms as well as isotopically labeled forms of the compounds. These forms of compounds may also be referred to as “isotopically enriched analogs.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine, and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I, and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The term “isotopically enriched analogs” includes “deuterated analogs” of compounds described herein in which one or more hydrogens is/are replaced by deuterium, such as a hydrogen on a carbon atom. Such compounds exhibit increased resistance to metabolism and are thus useful for increasing the half-life of any compound when administered to a mammal, particularly a human. See, for example, Foster, “Deuterium Isotope Effects in Studies of Drug Metabolism,” Trends Pharmacol. Sci. 5(12):524-527 (1984). Such compounds are synthesized by means well known in the art, for example by employing starting materials in which one or more hydrogens have been replaced by deuterium.

Deuterium labelled or substituted therapeutic compounds of the disclosure may have improved DMPK (drug metabolism and pharmacokinetics) properties, relating to distribution, metabolism, and excretion (ADME). Substitution with heavier isotopes such as deuterium may afford certain therapeutic advantages resulting from greater metabolic stability, for example increased in vivo half-life, reduced dosage requirements, and/or an improvement in therapeutic index. An ¹⁸F, ³H, or ¹¹C labeled compound may be useful for PET or SPECT or other imaging studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. It is understood that deuterium in this context is regarded as a substituent in a compound described herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

In many cases, the compounds of this disclosure are capable of forming acid and/or base salts by virtue of the presence of amino, and/or carboxyl groups, or groups similar thereto.

Provided are also a pharmaceutically acceptable salt, isotopically enriched analog, deuterated analog, stereoisomer, mixture of stereoisomers, and prodrugs of the compounds described herein. “Pharmaceutically acceptable” or “physiologically acceptable” refer to compounds, salts, compositions, dosage forms, and other materials which are useful in preparing a pharmaceutical composition that is suitable for veterinary or human pharmaceutical use.

The term “pharmaceutically acceptable salt” of a given compound refers to salts that retain the biological effectiveness and properties of the given compound and which are not biologically or otherwise undesirable. “Pharmaceutically acceptable salts” or “physiologically acceptable salts” include, for example, salts with inorganic acids, and salts with organic acids. In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare nontoxic pharmaceutically acceptable addition salts.

Pharmaceutically acceptable acid addition salts may be prepared from inorganic or organic acids. Salts derived from inorganic acids include, e.g., hydrochloric acid, hydrobromic acid, sulfuric acid, nitric acid, phosphoric acid, and the like. Salts derived from organic acids include, e.g., acetic acid, propionic acid, gluconic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Likewise, pharmaceutically acceptable base addition salts can be prepared from inorganic or organic bases. Salts derived from inorganic bases include, by way of example only, sodium, potassium, lithium, aluminum, ammonium, calcium, and magnesium salts. Salts derived from organic bases include, but are not limited to, salts of primary, secondary, and tertiary amines, such as alkyl amines (i.e., NH₂(alkyl)), dialkyl amines (i.e., HN(alkyl)₂), trialkyl amines (i.e., N(alkyl)₃), substituted alkyl amines (i.e., NH₂(substituted alkyl)), di(substituted alkyl) amines (i.e., HN(substituted alkyl)₂), tri(substituted alkyl) amines (i.e., N(substituted alkyl)₃), alkenyl amines (i.e., NH₂(alkenyl)), dialkenyl amines (i.e., HN(alkenyl)₂), trialkenyl amines (i.e., N(alkenyl)₃), substituted alkenyl amines (i.e., NH₂(substituted alkenyl)), di(substituted alkenyl) amines (i.e., HN(substituted alkenyl)₂), tri(substituted alkenyl) amines (i.e., N(substituted alkenyl)₃, mono-, di- or tri-cycloalkyl amines (i.e., NH₂(cycloalkyl), HN(cycloalkyl)₂, N(cycloalkyl)₃), mono-, di- or tri-arylamines (i.e., NH₂(aryl), HN(aryl)₂, N(aryl)₃), or mixed amines, etc. Specific examples of suitable amines include, by way of example only, isopropylamine, trimethyl amine, diethyl amine, tri(iso-propyl) amine, tri(n-propyl) amine, ethanolamine, 2-dimethylaminoethanol, piperazine, piperidine, morpholine, N-ethylpiperidine, and the like.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

The compounds of the disclosure, or their pharmaceutically acceptable salts include an asymmetric center and may thus give rise to enantiomers, diastereomers, and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents, or resolved using conventional techniques, for example, chromatography and/or fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers.

A “stereoisomer” refers to a compound made up of the same atoms bonded by the same bonds but having different three-dimensional structures, which are not interchangeable. The present disclosure contemplates various stereoisomers, or mixtures thereof, and includes “enantiomers,” which refers to two stereoisomers whose molecules are nonsuperimposable mirror images of one another.

“Diastereomers” are stereoisomers that have at least two asymmetric atoms, but which are not mirror-images of each other.

Relative centers of the compounds as depicted herein are indicated graphically using the “thick bond” style (bold or parallel lines) and absolute stereochemistry is depicted using wedge bonds (bold or parallel lines).

“Prodrugs” means any compound which releases an active parent drug according to a structure described herein in vivo when such prodrug is administered to a mammalian subject. Prodrugs of a compound described herein are prepared by modifying functional groups present in the compound described herein in such a way that the modifications may be cleaved in vivo to release the parent compound. Prodrugs may be prepared by modifying functional groups present in the compounds in such a way that the modifications are cleaved, either in routine manipulation or in vivo, to the parent compounds. Prodrugs include compounds described herein wherein a hydroxy, amino, carboxyl, or sulfhydryl group in a compound described herein is bonded to any group that may be cleaved in vivo to regenerate the free hydroxy, amino, or sulfhydryl group, respectively. Examples of prodrugs include, but are not limited to esters (e.g., acetate, formate, and benzoate derivatives), amides, guanidines, carbamates (e.g., N,N-dimethylaminocarbonyl) of hydroxy functional groups in compounds described herein, and the like. Preparation, selection, and use of prodrugs is discussed in T. Higuchi and V. Stella, “Pro-drugs as Novel Delivery Systems,” Vol. 14 of the A.C.S. Symposium Series; “Design of Prodrugs,” ed. H. Bundgaard, Elsevier, 1985; and in Bioreversible Carriers in Drug Design, ed. Edward B. Roche, American Pharmaceutical Association and Pergamon Press, 1987, each of which are hereby incorporated by reference in their entirety.

### 2. Compounds

Provided herein are compounds that are inhibitors of SARM1. In certain embodiments, provided is a compound of Formula I:

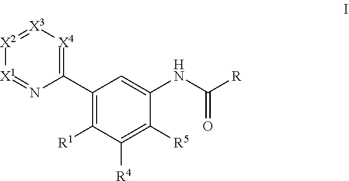

or a pharmaceutically acceptable salt, isotopically enriched analog, stereoisomer, tautomer, mixture of stereoisomers thereof, wherein:
each of X¹, X², X³, and X⁴ are independently N or CR⁶; provided no more than two of X¹, X², X³, and X⁴ are N;
R is tert-butyl, C₁₋₆ alkyl substituted with one to five R⁸, —NR²R³, —O—R⁷, C₃₋₁₀ cycloalkyl, or heterocyclyl; wherein the C₃₋₁₀ cycloalkyl or heterocyclyl is independently optionally substituted with one to five Z¹;
R¹ is halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R² is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R³ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
or R² and R³ together form a heterocyclyl, which may further be independently optionally substituted with one to five Z¹;
R⁴ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R⁵ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁶ is independently hydrogen, halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R⁷ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁸ is independently halo, cyano, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₃₋₁₀ cycloalkyl, heterocyclyl, aryl, or heteroaryl is independently optionally substituted with one to five Z¹;
each R¹¹ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z¹ is independently halo, cyano, —NO₂, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, —N(R¹²)₂, —OR¹², —SR¹², —C(O)R¹², —C(O)OR¹², —S(O)R¹², —S(O)₂R¹², —C(O)N(R¹²)₂, —NR¹²C(O)R¹², —NR¹²S(O)R¹², —NR¹²S(O)₂R¹², —S(O)N(R¹²)₂, —S(O)₂N(R¹²)₂, —NR¹²C(O)N(R¹²)₂, —NR¹²S(O)N(R¹²)₂, —NR¹²S(O)₂N(R¹²)₂, —OC(O)N(R¹²)₂, or —NR¹²C(O)OR¹²; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with one to five Z^{1a};
each R¹² is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1a} is independently halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —SR¹³, —C(O)R¹³, —C(O)OR¹³, —S(O)R¹³, —S(O)₂R¹³, —C(O)N(R¹³)₂, —NR¹³C(O)R¹³, —NR¹³S(O)R¹³, —NR¹³S(O)₂R¹³, —S(O)N(R¹³)₂, —S(O)₂N(R¹³)₂, —NR¹³C(O)N(R¹³)₂, —NR¹³S(O)N(R¹³)₂, —NR¹³S(O)₂N(R¹³)₂, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1b} is independently halo, cyano, —OH, —SH, —NH₂, —NO₂, C₁₋₆ alkyl, C₁₋₆ haloalkyl, C₂₋₆ alkenyl, C₂₋₆ alkynyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five halo, cyano, —OH, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl.

In certain embodiments, the compound is not N-[3-[6-ethoxy-5-(tetrahydro-2H-pyran-4-yl)-3-pyridazinyl]-4-methylphenyl]-2,3-dihydro-1H-isoindole-5-carboxamide (CAS No. 1887220-28-1), (3R)-1-[2-[4-(4-acetylphenyl)-1-piperazinyl]-2-oxoethyl]-N-[4-hydroxy-3-(2-pyridinyl)phenyl]-3-pyrrolidinecarboxamide (CAS No. 947607-02-5), 2,3-dihydro-5-methoxy-N-[4-methyl-3-(2-pyridinyl)phenyl]-6-(trifluoromethyl)-1H-indole-1-carboxamide (CAS No. 1349207-35-7), N-[3-[3-chloro-5-(trifluoromethyl)-2-pyridinyl]-4-methoxyphenyl]hexahydro-1,3-dimethyl-4,6-dioxo-2-thioxo-5-pyrimidinecarboxamide (CAS No. 102767-13-5), 1-(1,3-benzodioxol-5-yl)-N-[4-methyl-3-(2-pyridinyl)phenyl]-cyclopropanecarboxamide (CAS No. 945235-04-1), N-[4-[(4-propyl-1-piperazinyl)carbonyl]-3-(2-pyridinyl)phenyl]cyclopropanecarboxamide (CAS No. 2736488-37-0), dibutyl 4,4′-(((2-(pyridin-2-yl)-1,4-phenylene)bis(azanediyl))bis(carbonyl))bis(cyclohexane-1-carboxylate) (CAS No. 1443361-68-9), N-[3-(2-amino-4-pyrimidinyl)-4-fluorophenyl]-1-pyrrolidinecarboxamide (CAS No. 2080411-18-1), N-[3-(3-amino-1,2,4-triazin-5-yl)-4-fluorophenyl]-1-pyrrolidinecarboxamide (CAS No. 2080411-25-0), rel-N-[3,4-difluoro-5-[6-[[(3aR,6aS)-octahydro-2-[(tetrahydro-2H-pyran-4-yl)methyl]cyclopenta[c]pyrrol-5-yl]amino]-3-pyridazinyl]phenyl]-1-hydroxycyclobutanecarboxamide (CAS No. 2850202-28-5), rel-N-[3,4-difluoro-5-[6-[[(3aR,6aS)-octahydro-2-[(tetrahydro-2H-pyran-4-yl)methyl]cyclopenta[c]pyrrol-5-yl]amino]-3-pyridazinyl]phenyl]-3,3-difluorocyclobutanecarboxamide (CAS No. 2850202-29-6), rel-N-[3,4-difluoro-5-[6-[[(3aR,6aS)-octahydro-2-[(tetrahydro-2H-pyran-4-yl)methyl]cyclopenta[c]pyrrol-5-yl]amino]-3-pyridazinyl]phenyl]-1-fluorocyclopropanecarboxamide (CAS No. 2850202-27-4), or rel-N-[3,4-difluoro-5-[6-[[(3aR,6aS)-octahydro-2-[(tetrahydro-2H-pyran-4-yl)methyl]cyclopenta[c]pyrrol-5-yl]amino]-3-pyridazinyl]phenyl]-1-(trifluoromethyl)cyclopropanecarboxamide (CAS No. 2850202-26-3).

In certain embodiments, when X² is N, R¹ is methyl, R⁴ is hydrogen or fluoro, and R⁵ is hydrogen, then neither of X¹ and X³ are C-morpholino. In other words, in certain embodiments, when X² is N, R¹ is methyl, R⁴ is hydrogen or fluoro, and R⁵ is hydrogen, then neither of X¹ and X³ are CR⁶ where R⁶ is morpholino.

In certain embodiments, when R is —O—R⁷, then R¹ is methyl.

In certain embodiments, when R is substituted C₁₋₆ alkyl, then R¹ is substituted or unsubstituted C₁₋₆ alkyl.

In certain embodiments, when R is substituted or unsubstituted heterocyclyl, the substituted or unsubstituted heterocyclyl is other than a substituted or unsubstituted pyridin-2(1H)-onyl.

In certain embodiments, the moiety

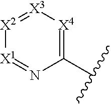

is not 4,5-diethyl-6-hydroxypyrimidine-2-yl or 4,5-diethyl-1,6-dihydro-6-oxo-2-pyrimidinyl.

In certain embodiments, provided is a compound of Formula I:

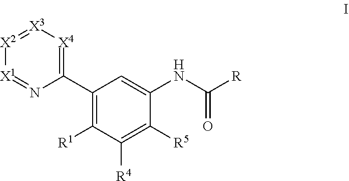

or a pharmaceutically acceptable salt, isotopically enriched analog, stereoisomer, tautomer, mixture of stereoisomers thereof, wherein:
each of X¹, X², X³, and X⁴ are independently N or CR⁶; provided no more than two of X¹, X², X³, and X⁴ are N;
R is tert-butyl, C₁₋₆ alkyl substituted with one to five R⁸, —NR²R³, —O—R⁷, C₃₋₁₀ cycloalkyl, or heterocyclyl; and the C₃₋₁₀ cycloalkyl or heterocyclyl is independently optionally substituted with one to five Z¹;
R¹ is halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R² is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R³ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
or R² and R³ together form a heterocyclyl, which may further be independently optionally substituted with one to five Z¹;
R⁴ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R⁵ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁶ is independently hydrogen, halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R⁷ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁸ is independently halo, cyano, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₃₋₁₀ cycloalkyl, heterocyclyl, aryl, or heteroaryl is independently optionally substituted with one to five Z¹;
each R¹¹ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z¹ is independently halo, cyano, —NO₂, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, —N(R¹²)₂, —OR¹², —SR¹², —C(O)R¹², —C(O)OR¹², —S(O)R¹², —S(O)₂R¹², —C(O)N(R¹²)₂, —NR¹²C(O)R¹², —NR¹²S(O)R¹², —NR¹²S(O)₂R¹², —S(O)N(R¹²)₂, —S(O)₂N(R¹²)₂, —NR¹²C(O)N(R¹²)₂, —NR¹²S(O)N(R¹²)₂, —NR¹²S(O)₂N(R¹²)₂, —OC(O)N(R¹²)₂, or —NR¹²C(O)OR¹²; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with one to five Z^{1a};
each R¹² is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1a} is independently halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —SR¹³, —C(O)R¹³, —C(O)OR¹³, —S(O)R¹³, —S(O)₂R¹³, —C(O)N(R¹³)₂, —NR¹³C(O)R¹³, —NR¹³S(O)R¹³, —NR¹³S(O)₂R¹³, —S(O)N(R¹³)₂, —S(O)₂N(R¹³)₂, —NR¹³C(O)N(R¹³)₂, —NR¹³S(O)N(R¹³)₂, —NR¹³S(O)₂N(R¹³)₂, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1b} is independently halo, cyano, —OH, —SH, —NH₂, —NO₂, C₁₋₆ alkyl, C₁₋₆ haloalkyl, C₂₋₆ alkenyl, C₂₋₆ alkynyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five halo, cyano, —OH, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl;
provided that:
i) the compound is not N-[3-[6-ethoxy-5-(tetrahydro-2H-pyran-4-yl)-3-pyridazinyl]-4-methylphenyl]-2,3-dihydro-1H-isoindole-5-carboxamide, (3R)-1-[2-[4-(4-acetylphenyl)-1-piperazinyl]-2-oxoethyl]-N-[4-hydroxy-3-(2-pyridinyl)phenyl]-3-pyrrolidinecarboxamide, 2,3-dihydro-5-methoxy-N-[4-methyl-3-(2-pyridinyl)phenyl]-6-(trifluoromethyl)-1H-indole-1-carboxamide, N-[3-[3-chloro-5-(trifluoromethyl)-2-pyridinyl]-4-methoxyphenyl]hexahydro-1,3-dimethyl-4,6-dioxo-2-thioxo-5-pyrimidinecarboxamide, (3R,5aS,6R,8aS,9S,11R,11aR)—N-[4-chloro-3-(2-pyridinyl)phenyl]octahydro-3,6,9-trimethyl-3,11-epoxy-3H,11H-furo[3,4-j]-1,2-benzodioxepin-9-carboxamide, 1-(1,3-benzodioxol-5-yl)-N-[4-methyl-3-(2-pyridinyl)phenyl]-cyclopropanecarboxamide, N-[4-[(4-propyl-1-piperazinyl)carbonyl]-3-(2-pyridinyl)phenyl]-cyclopropanecarboxamide, dibutyl 4,4′-(((2-(pyridin-2-yl)-1,4-phenylene)bis(azanediyl))bis(carbonyl))bis(cyclohexane-1-carboxylate), N-[3-(2-amino-4-pyrimidinyl)-4-fluorophenyl]-1-pyrrolidinecarboxamide, N-[3-(3-amino-1,2,4-triazin-5-yl)-4-fluorophenyl]-1-pyrrolidinecarboxamide, 2-[4-methyl-6-(4-morpholinyl)-1,3,5-triazin-2-yl]-4-[(4-morpholinylcarbonyl)amino]benzenesulfonic acid, N-[4-fluoro-3-[2-[3-(hydroxymethyl)phenyl]-6-(4-morpholinyl)-4-pyrimidinyl]phenyl]-1-piperazinecarboxamide, 2,3-dihydro-5-methoxy-N-[4-methyl-3-(2-pyridinyl)phenyl]-6-(trifluoromethyl)-1H-indole-1-carboxamide, rel-N-[3,4-difluoro-5-[6-[[(3aR,6aS)-octahydro-2-[(tetrahydro-2H-pyran-4-yl)methyl]cyclopenta[c]pyrrol-5-yl]amino]-3-pyridazinyl]phenyl]-1-hydroxycyclobutanecarboxamide, rel-N-[3,4-difluoro-5-[6-[[(3aR,6aS)-octahydro-2-[(tetrahydro-2H-pyran-4-yl)methyl]cyclopenta[c]pyrrol-5-yl]amino]-3-pyridazinyl]phenyl]-3,3-difluorocyclobutanecarboxamide, rel-N-[3,4-difluoro-5-[6-[[(3aR,6aS)-octahydro-2-[(tetrahydro-2H-pyran-4-yl)methyl]cyclopenta[c]pyrrol-5-yl]amino]-3-pyridazinyl]phenyl]-1-fluorocyclopropanecarboxamide, or rel-N-[3,4-difluoro-5-[6-[[(3aR,6aS)-octahydro-2-[(tetrahydro-2H-pyran-4-yl)methyl]cyclopenta[c]pyrrol-5-yl]amino]-3-pyridazinyl]phenyl]-1-(trifluoromethyl)cyclopropanecarboxamide;
ii) when X² is N, R¹ is methyl, R⁴ is hydrogen or fluoro, and R⁵ is hydrogen, then neither of X¹ and X³ are C-morpholino;
iii) when R is —O—R⁷, then R¹ is methyl;
iv) when R is substituted C₁₋₆ alkyl, then R¹ is substituted or unsubstituted C₁₋₆ alkyl; and
v) when R is substituted or unsubstituted heterocyclyl, the substituted or unsubstituted heterocyclyl is other than a substituted or unsubstituted pyridin-2(1H)-onyl.

In certain embodiments, provided is a compound of Formula I:

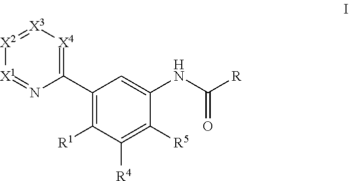

or a pharmaceutically acceptable salt, isotopically enriched analog, stereoisomer, tautomer, mixture of stereoisomers thereof, wherein:
each of X¹, X², X³, and X⁴ are independently N or CR⁶; provided no more than two of X¹, X², X³, and X⁴ are N;
R is tert-butyl, C₁₋₆ alkyl, —NR²R³, —O—R⁷, C₃₋₁₀ cycloalkyl, or heterocyclyl; wherein the C₁₋₆ alkyl is substituted with one to five R⁸, and the C₃₋₁₀ cycloalkyl or heterocyclyl is independently optionally substituted with one to five Z¹;
R¹ is halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R² is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R³ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
or R² and R³ together form a heterocyclyl, which may further be independently optionally substituted with one to five Z¹;
R⁴ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R⁵ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁶ is independently hydrogen, halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R⁷ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁸ is independently halo, cyano, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₃₋₁₀ cycloalkyl, heterocyclyl, aryl, or heteroaryl is independently optionally substituted with one to five Z¹;
each R¹¹ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z¹ is independently halo, cyano, —NO₂, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, —N(R¹²)₂, —OR¹², —SR¹², —C(O)R¹², —C(O)OR¹², —S(O)R¹², —S(O)₂R¹², —C(O)N(R¹²)₂, —NR¹²C(O)R¹², —NR¹²S(O)R¹², —NR¹²S(O)₂R¹², —S(O)N(R¹²)₂, —S(O)₂N(R¹²)₂, —NR¹²C(O)N(R¹²)₂, —NR¹²S(O)N(R¹²)₂, —NR¹²S(O)₂N(R¹²)₂, —OC(O)N(R¹²)₂, or —NR¹²C(O)OR¹²; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with one to five Z^{1a};
each R¹² is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1a} is independently halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —SR¹³, —C(O)R¹³, —C(O)OR¹³, —S(O)R¹³, —S(O)₂R¹³, —C(O)N(R¹³)₂, —NR¹³C(O)R¹³, —NR¹³S(O)R¹³, —NR¹³S(O)₂R¹³, —S(O)N(R¹³)₂, —S(O)₂N(R¹³)₂, —NR¹³C(O)N(R¹³)₂, —NR¹³S(O)N(R¹³)₂, —NR¹³S(O)₂N(R¹³)₂, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1b} is independently halo, cyano, —OH, —SH, —NH₂, —NO₂, C₁₋₆ alkyl, C₁₋₆ haloalkyl, C₂₋₆ alkenyl, C₂₋₆ alkynyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five halo, cyano, —OH, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl;
provided that:
i) the compound is not N-[3-[6-ethoxy-5-(tetrahydro-2H-pyran-4-yl)-3-pyridazinyl]-4-methylphenyl]-2,3-dihydro-1H-isoindole-5-carboxamide, (3R)-1-[2-[4-(4-acetylphenyl)-1-piperazinyl]-2-oxoethyl]-N-[4-hydroxy-3-(2-pyridinyl)phenyl]-3-pyrrolidinecarboxamide, 2,3-dihydro-5-methoxy-N-[4-methyl-3-(2-pyridinyl)phenyl]-6-(trifluoromethyl)-1H-indole-1-carboxamide, N-[3-[3-chloro-5-(trifluoromethyl)-2-pyridinyl]-4-methoxyphenyl]hexahydro-1,3-dimethyl-4,6-dioxo-2-thioxo-5-pyrimidinecarboxamide, (3R,5aS,6R,8aS,9S,11R,11aR)—N-[4-chloro-3-(2-pyridinyl)phenyl]octahydro-3,6,9-trimethyl-3,11-epoxy-3H,11H-furo[3,4-j]-1,2-benzodioxepin-9-carboxamide, 1-(1,3-benzodioxol-5-yl)-N-[4-methyl-3-(2-pyridinyl)phenyl]-cyclopropanecarboxamide, N-[4-[(4-propyl-1-piperazinyl)carbonyl]-3-(2-pyridinyl)phenyl]-cyclopropanecarboxamide, dibutyl 4,4′-(((2-(pyridin-2-yl)-1,4-phenylene)bis(azanediyl))bis(carbonyl))bis(cyclohexane-1-carboxylate), N-[3-(2-amino-4-pyrimidinyl)-4-fluorophenyl]-1-pyrrolidinecarboxamide, N-[3-(3-amino-1,2,4-triazin-5-yl)-4-fluorophenyl]-1-pyrrolidinecarboxamide, 2-[4-methyl-6-(4-morpholinyl)-1,3,5-triazin-2-yl]-4-[(4-morpholinylcarbonyl)amino]benzenesulfonic acid, N-[4-fluoro-3-[2-[3-(hydroxymethyl)phenyl]-6-(4-morpholinyl)-4-pyrimidinyl]phenyl]-1-piperazinecarboxamide, or 2,3-dihydro-5-methoxy-N-[4-methyl-3-(2-pyridinyl)phenyl]-6-(trifluoromethyl)-1H-indole-1-carboxamide;
ii) when X² is N, R¹ is methyl, R⁴ is hydrogen or fluoro, and R⁵ is hydrogen, then neither of X¹ and X³ are C-morpholino;
iii) when R is —O—R⁷, then R¹ is methyl;
iv) when R is substituted C₁₋₆ alkyl, then R¹ is substituted or unsubstituted C₁₋₆ alkyl; and
v) when R is substituted or unsubstituted heterocyclyl, the substituted or unsubstituted heterocyclyl is other than a substituted or unsubstituted pyridin-2(1H)-onyl.

In certain embodiments, R is tert-butyl, C₁₋₆ alkyl substituted with one to five R⁸, C₃₋₁₀ cycloalkyl, or heterocyclyl; wherein C₃₋₁₀ cycloalkyl or heterocyclyl is independently optionally substituted with one to five Z¹.

In certain embodiments, R is tert-butyl or C₁₋₆ alkyl substituted with one to five R⁸.

In certain embodiments, R is tert-butyl.

In certain embodiments, R is C₁₋₆ alkyl substituted with one to five R⁸.

In certain embodiments, each R⁸ is independently halo, C₃₋₁₀ cycloalkyl, heterocyclyl, or —OR¹¹.

In certain embodiments, each R⁸ is independently halo, C₃₋₁₀ cycloalkyl, heterocyclyl, or —OH.

In certain embodiments, R is C₃₋₁₀ cycloalkyl or heterocyclyl; wherein the C₃₋₁₀ cycloalkyl or heterocyclyl is independently optionally substituted with one to five Z¹.

In certain embodiments, R is C₃₋₁₀ cycloalkyl optionally substituted with one to five Z¹.

In certain embodiments, R is heterocyclyl optionally substituted with one to five Z¹.

In certain embodiments, the compound is represented by Formula II:

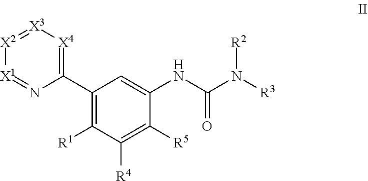

wherein each of X¹, X², X³, X⁴, R¹, R², R³, R⁴, and R⁵ are independently as defined herein.

In certain embodiments, provided is a compound of Formula II:

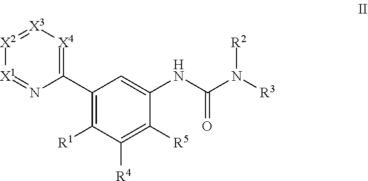

or a pharmaceutically acceptable salt, isotopically enriched analog, stereoisomer, tautomer, mixture of stereoisomers thereof, wherein:
each of X¹, X², X³, and X⁴ are independently N or CR⁶; provided no more than two of X¹, X², X³, and X⁴ are N;
R¹ is halo, cyano, —NO₂, C₁₋₆ alkyl, C₂₋₆alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R² is C₁₋₆ alkyl, C₂₋₆alkenyl, C₂₋₆alkynyl, C₃₋₁₀ cycloalkyl, heterocyclyl, aryl, or heteroaryl; wherein the C₁₋₆ alkyl, C₂₋₆alkenyl, C₂₋₆ alkynyl, C₃₋₁₀ cycloalkyl, heterocyclyl, aryl, or heteroaryl is independently optionally substituted with one to five Z¹;
R³ is C₁₋₆ alkyl, C₂₋₆alkenyl, C₂₋₆alkynyl, C₃₋₁₀ cycloalkyl, heterocyclyl, aryl, or heteroaryl; wherein the C₁₋₆ alkyl, C₂₋₆alkenyl, C₂₋₆ alkynyl, C₃₋₁₀ cycloalkyl, heterocyclyl, aryl, or heteroaryl is independently optionally substituted with one to five Z¹;
or R² and R³ together form a heterocyclyl, which may further be independently optionally substituted with one to five Z¹;
R⁴ is hydrogen, halo, cyano, C₁₋₆ alkyl, C₂₋₆alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R⁵ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁶ is independently hydrogen, halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
each R¹¹ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z¹ is independently halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹²)₂, —OR¹², —SR¹², —C(O)R¹², —C(O)OR¹², —S(O)R¹², —S(O)₂R¹², —C(O)N(R¹²)₂, —NR¹²C(O)R¹², —NR¹²S(O)R¹², —NR¹²S(O)₂R¹², —S(O)N(R¹²)₂, —S(O)₂N(R¹²)₂, —NR¹²C(O)N(R¹²)₂, —NR¹²S(O)N(R¹²)₂, —NR¹²S(O)₂N(R¹²)₂, —OC(O)N(R¹²)₂, or —NR¹²C(O)OR¹²; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each R¹² is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1a} is independently halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —SR¹³, —C(O)R¹³, —C(O)OR¹³, —S(O)R¹³, —S(O)₂R¹³, —C(O)N(R¹³)₂, —NR¹³C(O)R¹³, —NR³S(O)R¹³, —NR¹³S(O)₂R¹³, —S(O)N(R¹³)₂, —S(O)₂N(R¹³)₂, —NR¹³C(O)N(R¹³)₂, —NR¹³S(O)N(R¹³)₂, —NR¹³S(O)₂N(R¹³)₂, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1b} is independently halo, cyano, —OH, —SH, —NH₂, —NO₂, C₁₋₆ alkyl, C₁₋₆ haloalkyl, C₂₋₆ alkenyl, C₂₋₆ alkynyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five halo, cyano, —OH, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy,
C₁₋₆ haloalkoxy, C₃₋₁₀ cycloalkyl, heterocyclyl, aryl, or heteroaryl; provided that the compound is not N-[3-(2-amino-4-pyrimidinyl)-4-fluorophenyl]-1-pyrrolidinecarboxamide, N-[3-(3-amino-1,2,4-triazin-5-yl)-4-fluorophenyl]-1-pyrrolidinecarboxamide, 2-[4-methyl-6-(4-morpholinyl)-1,3,5-triazin-2-yl]-4-[(4-morpholinylcarbonyl)amino]benzenesulfonic acid, N-[4-fluoro-3-[2-[3-(hydroxymethyl)phenyl]-6-(4-morpholinyl)-4-pyrimidinyl]phenyl]-1-piperazinecarboxamide, or 2,3-dihydro-5-methoxy-N-[4-methyl-3-(2-pyridinyl)phenyl]-6-(trifluoromethyl)-1H-indole-1-carboxamide, and when X² is N, R¹ is methyl, R⁴ is hydrogen and R⁵ is hydrogen, then neither of X¹ and X³ are C-morpholino.

In certain embodiments, the compound is represented by Formula III:

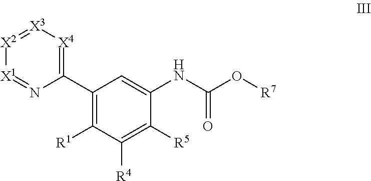

wherein each of X¹, X², X³, X⁴, R¹, R⁴, R⁵ and R⁷ are independently as defined herein.

In certain embodiments, R⁷ is C₁₋₆ alkyl.

In certain embodiments, X¹ is N.

In certain embodiments, X², X³, and X⁴ are CR⁶.

In certain embodiments, X² is N.

In certain embodiments, X¹, X³, and X⁴ are CR⁶.

In certain embodiments, X³ is N.

In certain embodiments, X¹, X², and X⁴ are CR⁶.

In certain embodiments, X⁴ is N.

In certain embodiments, X¹, X², and X³ are CR⁶.

In certain embodiments, X¹ and X³ are N.

In certain embodiments, X² and X⁴ are CR⁶.

In certain embodiments, X¹ and X⁴ are N.

In certain embodiments, X² and X³ are CR⁶.

In certain embodiments, X² and X⁴ are N.

In certain embodiments, X¹ and X³ are CR⁶.

In certain embodiments, X³ and X⁴ are N.

In certain embodiments, X¹ and X² are CR⁶.

In certain embodiments, R⁴ is hydrogen, fluoro, chloro, bromo, cyano, or methyl.

In certain embodiments, R⁴ is hydrogen.

In certain embodiments, R⁵ is hydrogen or fluoro.

In certain embodiments, R⁵ is hydrogen, fluoro, or chloro.

In certain embodiments, R⁴ and R⁵ are hydrogen. In certain embodiments, the compound is represented by Formula IA:

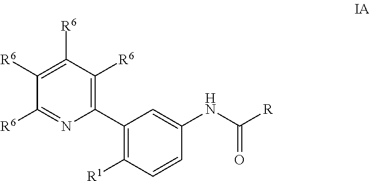

wherein each R, R¹, and R⁶ are independently as defined herein.

In certain embodiments, the compound is represented by Formula IIA:

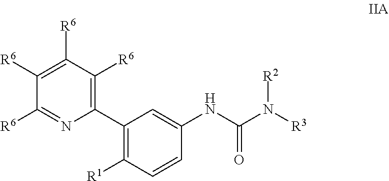

wherein each R¹, R², R³, and R⁶ are independently as defined herein.

In certain embodiments, the compound is represented by Formula IIIA:

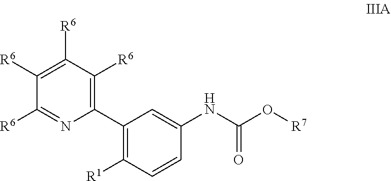

wherein each R¹, R⁶, and R⁷ are independently as defined herein.

In certain embodiments, the compound is represented by Formula IIB:

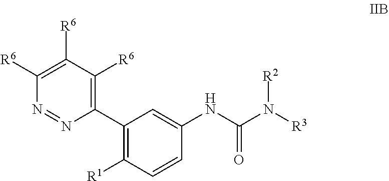

wherein each of R¹, R², R³ and R⁶ are independently as defined herein.

In certain embodiments, the compound is represented by Formula IIB-1:

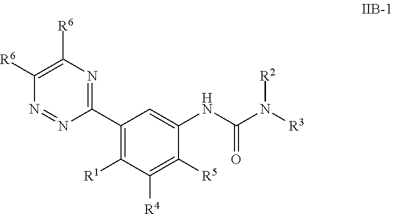

wherein each of R¹, R², R³ and R⁶ are independently as defined herein.

In certain embodiments, the compound is represented by Formula IIB-2:

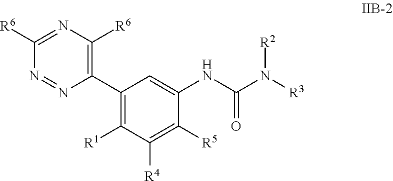

wherein each of R¹, R², R³ and R⁶ are independently as defined herein.

In certain embodiments, the compound is represented by Formula IIC:

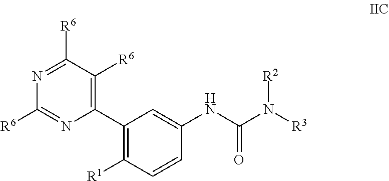

wherein each of R¹, R², R³ and R⁶ are independently as defined herein.

In certain embodiments, the compound is represented by Formula IID:

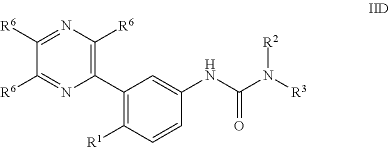

wherein each of R¹, R², R³ and R⁶ are independently as defined herein.

In certain embodiments, the compound is represented by Formula IIE:

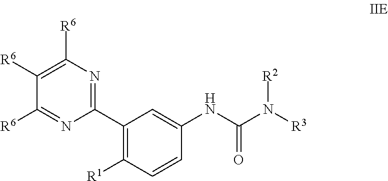

wherein each of R¹, R², R³ and R⁶ are independently as defined herein.

In certain embodiments, the compound is represented by Formula IIE:

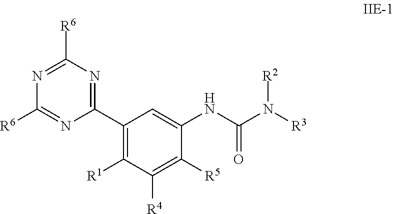

wherein each of R¹, R², R³ and R⁶ are independently as defined herein.

In certain embodiments, the compound is represented by Formula IIE:

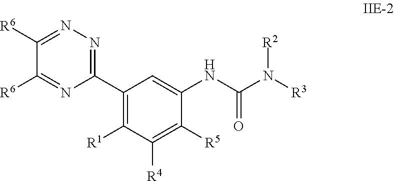

wherein each of R¹, R², R³ and R⁶ are independently as defined herein.

In certain embodiments, provided is a compound of Formula IV:

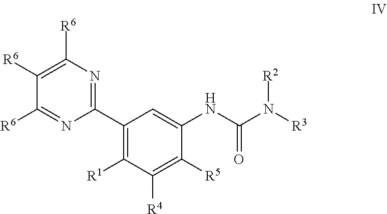

or a pharmaceutically acceptable salt, isotopically enriched analog, stereoisomer, tautomer, mixture of stereoisomers thereof, wherein:
R¹ is hydrogen, halo, cyano, —NO₂, C₁₋₆ alkyl, C₂₋₆alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆alkynyl, C₃₋₁₀ cycloalkyl, heterocyclyl, aryl, or heteroaryl, is independently optionally substituted with one to five Z¹;
R² is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R³ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
or R² and R³ together form a heterocyclyl, which may further be independently optionally substituted with one to five Z¹;
R⁴ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R⁵ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁶ is independently hydrogen, halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
each R¹¹ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z¹ is independently halo, cyano, —NO₂, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, —N(R¹²)₂, —OR¹², —SR¹², —C(O)R¹², —C(O)OR¹², —S(O)R¹², —S(O)₂R¹², —C(O)N(R¹²)₂, —NR¹²C(O)R¹², —NR¹²S(O)R¹², —NR¹²S(O)₂R¹², —S(O)N(R¹²)₂, —S(O)₂N(R¹²)₂, —NR¹²C(O)N(R¹²)₂, —NR¹²S(O)N(R¹²)₂, —NR¹²S(O)₂N(R¹²)₂, —OC(O)N(R¹²)₂, or —NR¹²C(O)OR¹²; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with one to five Z^{1a};
each R¹² is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1a} is independently halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —SR¹³, —C(O)R¹³, —C(O)OR¹³, —S(O)R¹³, —S(O)₂R¹³, —C(O)N(R¹³)₂, —NR¹³C(O)R¹³, —NR¹³S(O)R¹³, —NR¹³S(O)₂R¹³, —S(O)N(R¹³)₂, —S(O)₂N(R¹³)₂, —NR¹³C(O)N(R¹³)₂, —NR¹³S(O)N(R¹³)₂, —NR¹³S(O)₂N(R¹³)₂, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1b} is independently halo, cyano, —OH, —SH, —NH₂, —NO₂, C₁₋₆ alkyl, C₁₋₆ haloalkyl, C₂₋₆ alkenyl, C₂₋₆ alkynyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five halo, cyano, —OH, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl;
provided that:
a) the moiety

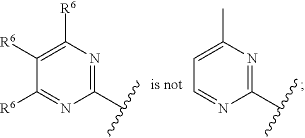

and
b) the compound is not N-[3-[4-[3-(hydroxymethyl)phenyl]-6-(4-morpholinyl)-2-pyrimidinyl]phenyl]-1-piperazinecarboxamide, N-[3-[4-(1H-indazol-5-ylamino)-2-pyrimidinyl]phenyl]-4-morpholinecarboxamide, N-[3-ethyl-5-(2-pyrimidinyl)phenyl]-7,8-dihydro-4-(2-methylphenyl)-2-(3-pyridinyl)pyrido[4,3-d]pyrimidine-6(5H)-carboxamide, or 1,1-dimethylethyl 5-[[(1,1-dimethylethoxy)carbonyl][2-[3-[(4-morpholinylcarbonyl)amino]phenyl]-4-pyrimidinyl]amino]-1H-indazole-1-carboxylate.

In certain embodiments, each R⁶ is independently hydrogen, halo, cyano, C₁₋₆ alkyl, C₁₋₆ haloalkyl, C₃₋₁₀ cycloalkyl, —O—C₁₋₆ alkyl, or —O—C₁₋₆ haloalkyl.

In certain embodiments, each R⁶ is independently hydrogen, halo, cyano, C₁₋₆ alkyl, C₁₋₆ haloalkyl, C₃₋₁₀ cycloalkyl, or —O—C₁₋₆ alkyl.

In certain embodiments, each R⁶ is independently hydrogen, fluoro, chloro, cyano, methyl, ethyl, trifluoromethyl, cyclopropyl, methoxy, 2-methoxyethoxymethyl, fluoromethoxy, or difluoromethoxy.

In certain embodiments, each R⁶ is independently hydrogen, fluoro, chloro, cyano, methyl, ethyl, trifluoromethyl, cyclopropyl, or methoxy.

In certain embodiments, each R⁶ is independently hydrogen, fluoro, cyano, methyl, trifluoromethyl, cyclopropyl, or methoxy.

In certain embodiments, no R⁶ is hydroxy.

In certain embodiments, R¹ is halo, cyano, C₁₋₆ alkyl, C₃₋₁₀ cycloalkyl, aryl, or —OR¹¹; wherein the C₁₋₆ alkyl, C₃₋₁₀ cycloalkyl, or aryl is optionally substituted with one to five Z¹.

In certain embodiments, R¹ is halo, cyano, C₁₋₆ alkyl, C₃₋₁₀ cycloalkyl, or —OR¹¹; wherein the C₁₋₆ alkyl or C₃₋₁₀ cycloalkyl is optionally substituted with one to five Z¹.

In certain embodiments, R¹ is fluoro, chloro, bromo, cyano, methyl, ethyl, isopropyl, —CH₂F, —CHF₂, —CF₃, —CH₂CN, —OCF₃, cyclopropyl optionally substituted with methyl, or phenyl.

In certain embodiments, R¹ is fluoro, chloro, cyano, methyl, ethyl, —CF₃, —CH₂CN, —OCF₃, or cyclopropyl.

In certain embodiments, R¹ is fluoro, chloro, cyano, methyl, ethyl, isopropyl, —CHF₂, —CF₃, —CH₂CN, —OCF₃, cyclopropyl optionally substituted with methyl, or phenyl.

In certain embodiments, R is C₁₋₆ alkyl substituted with one to five R⁸. In certain embodiments, R is C₁₋₂ alkyl substituted with one to five R⁸.

In certain embodiments, each R⁸ is independently halo, C₃₋₁₀ cycloalkyl, heterocyclyl, or —OR¹¹.

In certain embodiments, each R⁸ is independently halo, C₃₋₁₀ cycloalkyl, heterocyclyl, or —OH.

In certain embodiments, R is:

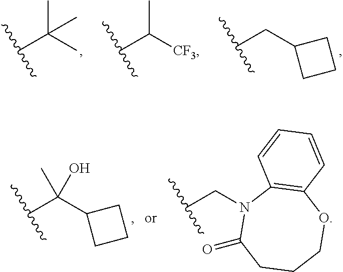

In certain embodiments, R is C₃₋₁₀ cycloalkyl optionally substituted with one to five Z¹. In certain embodiments, R is C₃₋₇ cycloalkyl optionally substituted with one to five Z¹.

In certain embodiments, R is:

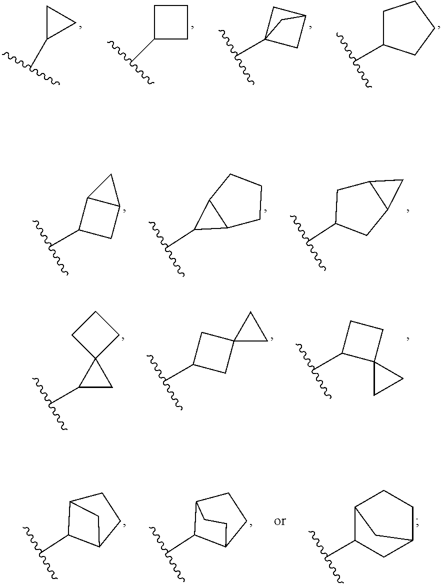

wherein each is independently optionally substituted with one to five Z¹.

In certain embodiments, R is:

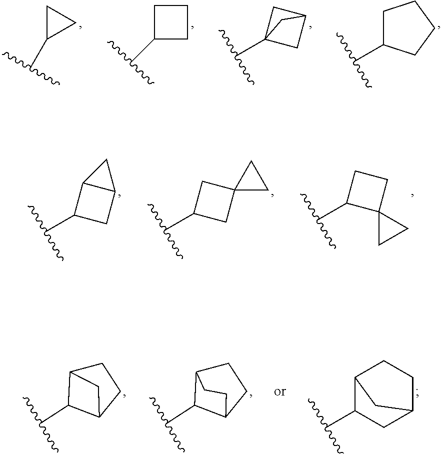

wherein each is independently optionally substituted with one to five Z¹.

In certain embodiments, each Z¹ is independently halo, C₁₋₆ alkyl, C₁₋₆ haloalkyl, C₁₋₆ haloalkoxy, or hydroxy.

In certain embodiments, R is:

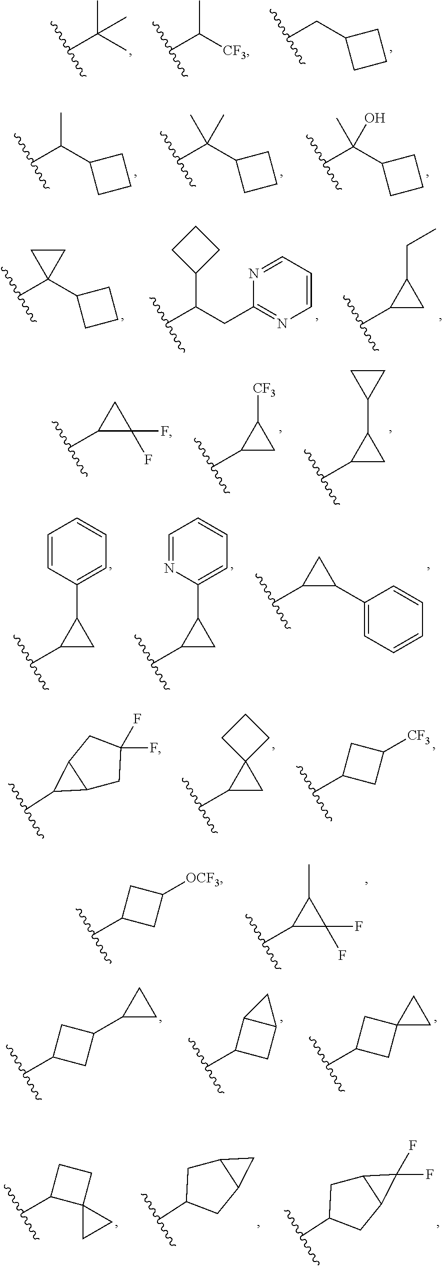

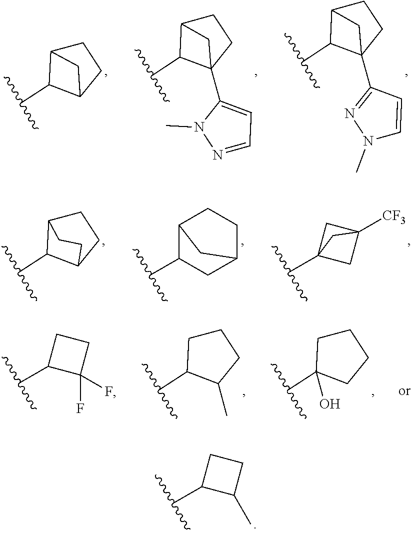

In certain embodiments, R is:

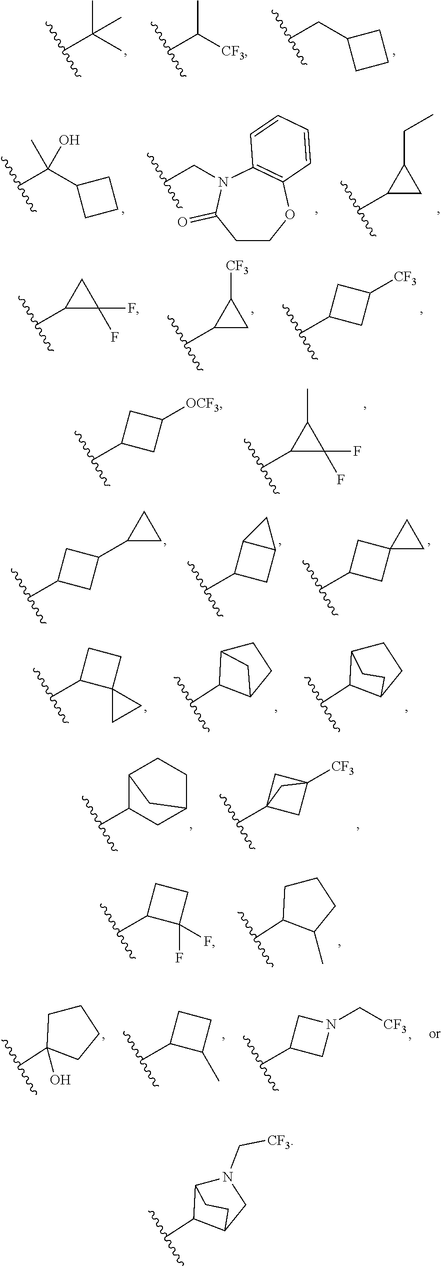

In certain embodiments, R is:

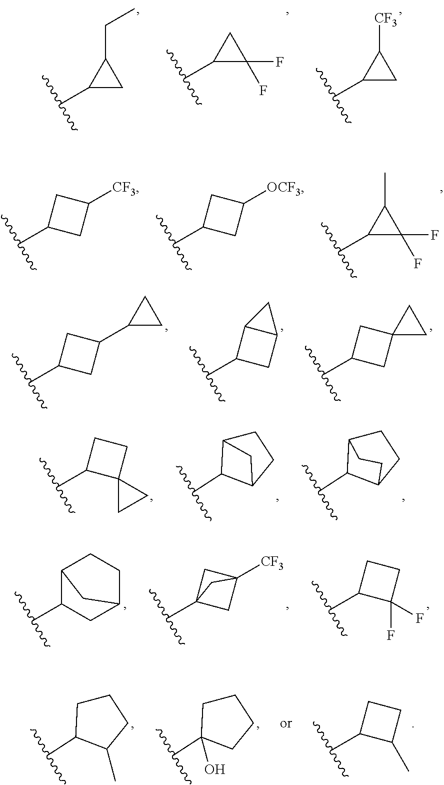

In certain embodiments, R is heterocyclyl optionally substituted with one to five Z¹. In certain embodiments, the heterocyclyl is bonded to the carbonyl moiety of the amide via a carbon atom.

In certain embodiments, R is a 4- to 7-membered heterocyclyl optionally substituted with one to five Z¹.

In certain embodiments, R is:

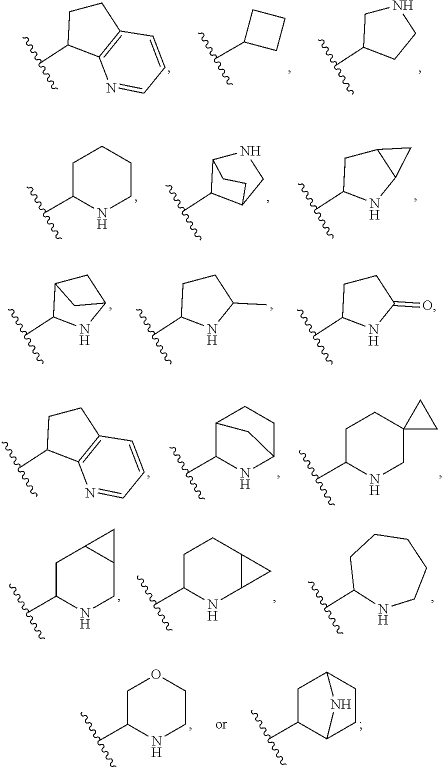

wherein each is independently optionally substituted with one to five Z¹.

In certain embodiments, R is:

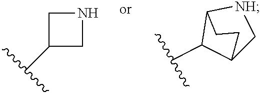 [SMILES: *C1CNC1 ; *C1C2CCC1NC2]

wherein each is independently optionally substituted with one to five Z¹.

In certain embodiments, each Z¹ is independently C₁₋₆alkyl, C₁₋₆ haloalkyl, —OR¹¹, —C(O)R¹¹, C₃₋₁₀ cycloalkyl, aryl, or heteroaryl.

In certain embodiments, each Z¹ is independently C₁₋₆ haloalkyl.

In certain embodiments, R is:

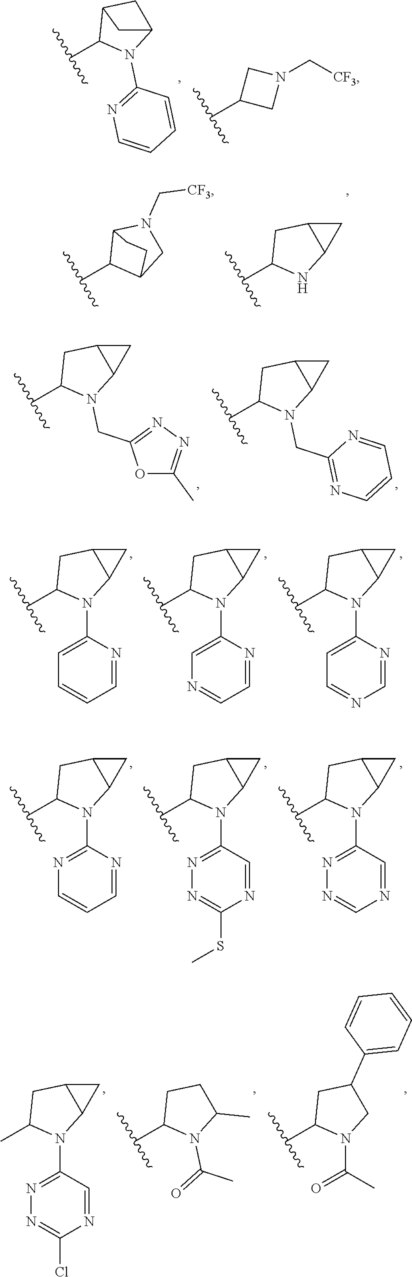

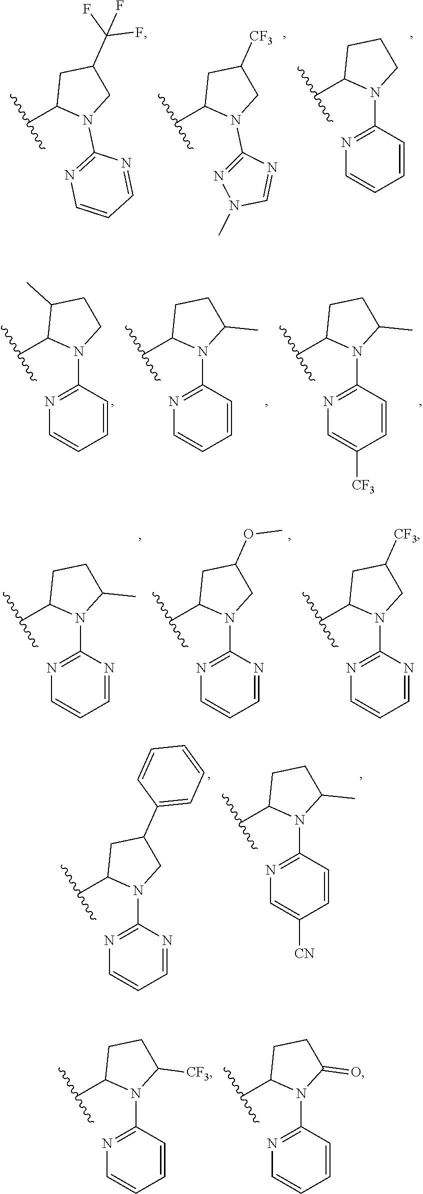

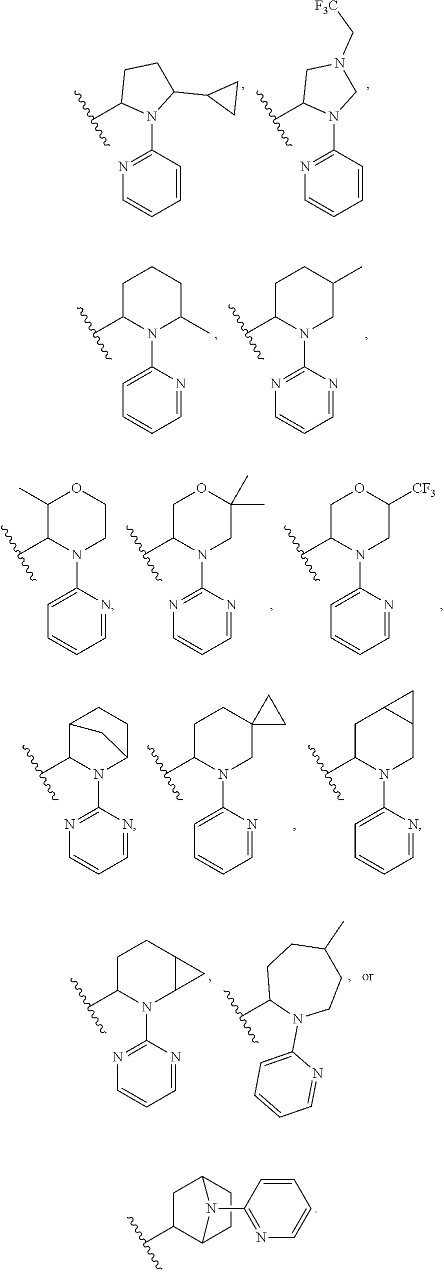

In certain embodiments, R is:

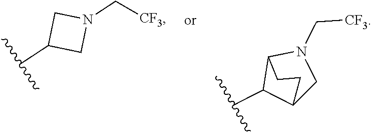 [SMILES: *C1CN(CC(F)(F)F)C1 ; *C1C2CCC1N(CC(F)(F)F)C2]

In certain embodiments, R is —O—R⁷, wherein R⁷ is C₁₋₆alkyl, optionally substituted with one to five Z¹.

In certain embodiments, Z1 is independently C₃₋₁₀ cycloalkyl or aryl.

In certain embodiments, R or the moiety

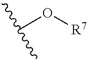

is:

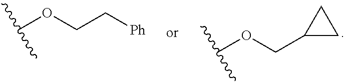 [SMILES: *OCCc1ccccc1 ; *OCC1CC1]

In certain embodiments, R or the moiety

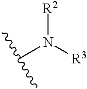

of the Formulas described herein is:

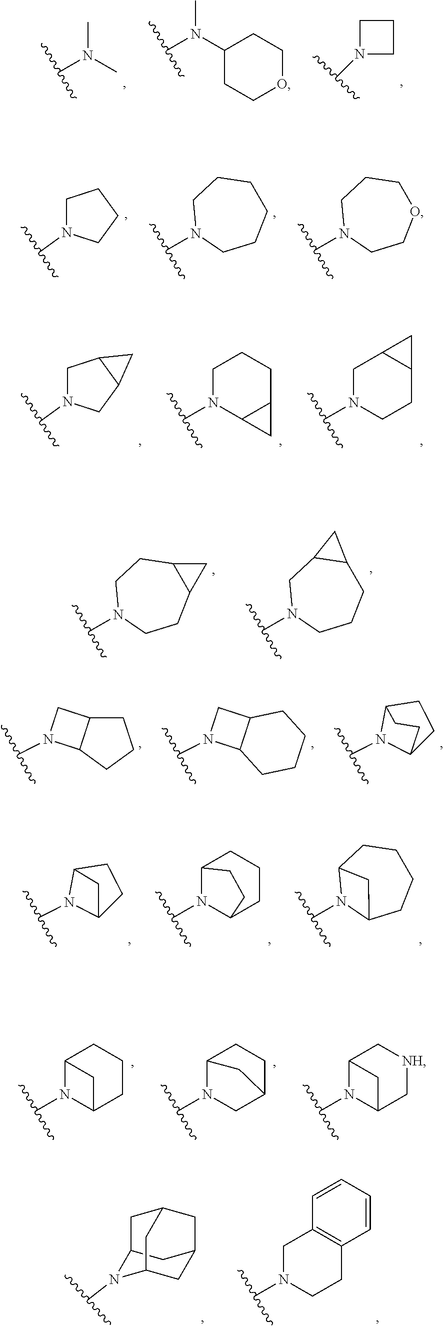

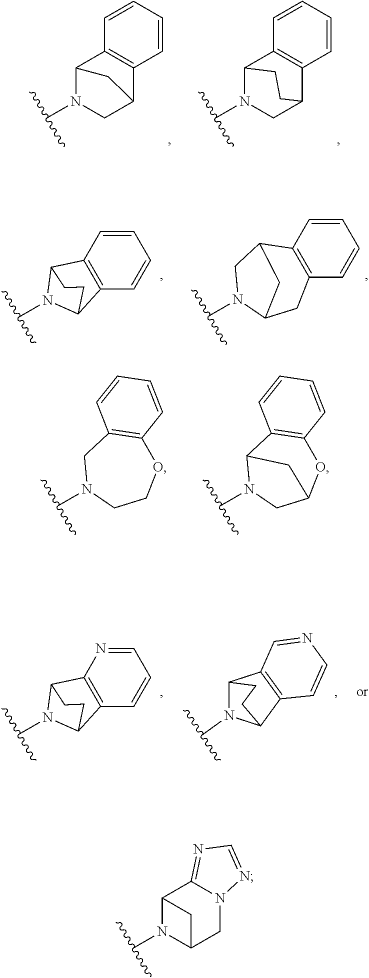

wherein each is independently optionally substituted with one to five Z¹.

In certain embodiments, R² and R³ together form a heterocyclyl, which may further be independently optionally substituted with one to five Z¹.

In certain embodiments, R² and R³ together form a heterocyclyl, which may further be independently optionally substituted with halo, —OH, —C(O)O—C₁₋₆ alkyl, —O—C₁₋₆ alkyl, —O—C₁₋₆haloalkyl, C₁₋₆ alkyl, C₁₋₆haloalkyl, C₃₋₁₀ cycloalkyl, aryl, or heteroaryl; wherein the C₁₋₆alkyl or heteroaryl is optionally substituted with one to five Z^{1a}.

n embodiments, R² and R² and R³ together form a heterocyclyl, which may further be independently optionally substituted with C₁₋₆ alkyl, C₁₋₆ haloalkyl, C₃₋₁₀ cycloalkyl, —OH, —O—C₁₋₆ alkyl, —O—C₁₋₆ haloalkyl, aryl, or —C(O)O—C₁₋₆ alkyl.

In certain embodiments, the compound is represented by Formula IIF:

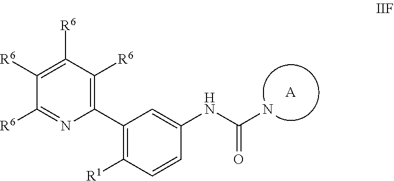

wherein ring A is a 4-15 membered heterocyclyl, which may further be independently optionally substituted with one to five Z¹, and each R¹ and R⁶ are independently as defined herein.

In certain embodiments, the compound is represented by Formula IIG:

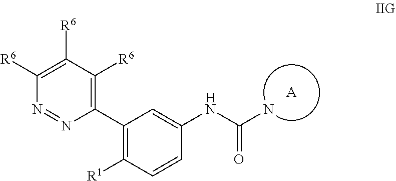

wherein ring A is a 4-15 membered heterocyclyl, which may further be independently optionally substituted with one to five Z¹, and each R¹ and R⁶ are independently as defined herein.

In certain embodiments, the compound is represented by Formula IIG-1:

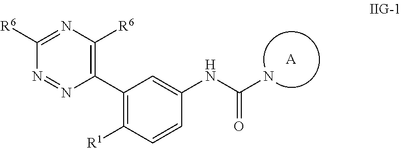

wherein ring A is a 4-15 membered heterocyclyl, which may further be independently optionally substituted with one to five Z¹, and each R¹ and R⁶ are independently as defined herein.

In certain embodiments, the compound is represented by Formula IIG-2:

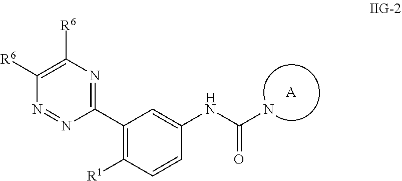

wherein ring A is a 4-15 membered heterocyclyl, which may further be independently optionally substituted with one to five Z¹, and each R¹ and R⁶ are independently as defined herein.

In certain embodiments, the compound is represented by Formula IIH:

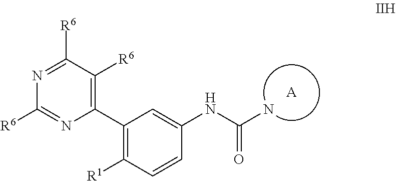

wherein ring A is a 4-15 membered heterocyclyl, which may further be independently optionally substituted with one to five Z¹, and each R¹ and R⁶ are independently as defined herein.

In certain embodiments, the compound is represented by Formula IIJ:

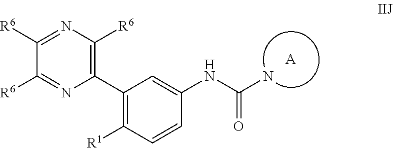

wherein ring A is a 4-15 membered heterocyclyl, which may further be independently optionally substituted with one to five Z¹, and each R¹ and R⁶ are independently as defined herein.

In certain embodiments, the compound is represented by Formula IIK:

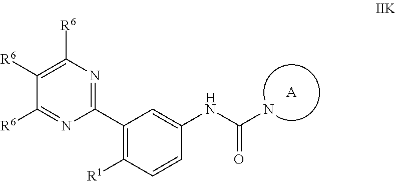

wherein ring A is a 4-15 membered heterocyclyl, which may further be independently optionally substituted with one to five Z¹, and each R¹ and R⁶ are independently as defined herein.

In certain embodiments, the compound is represented by Formula IIK:

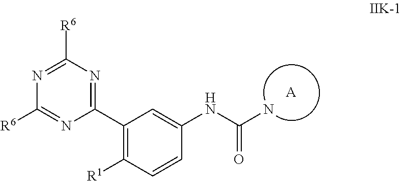

wherein ring A is a 4-15 membered heterocyclyl, which may further be independently optionally substituted with one to five Z¹, and each R¹ and R⁶ are independently as defined herein.

In certain embodiments, the compound is represented by Formula IIK:

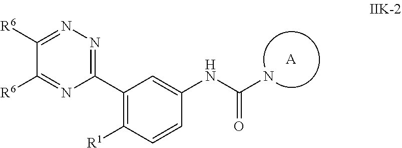

wherein ring A is a 4-15 membered heterocyclyl, which may further be independently optionally substituted with one to five Z¹, and each R¹ and R⁶ are independently as defined herein.

In certain embodiments, R, the moiety

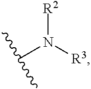

or ring A of the Formulas described herein is:

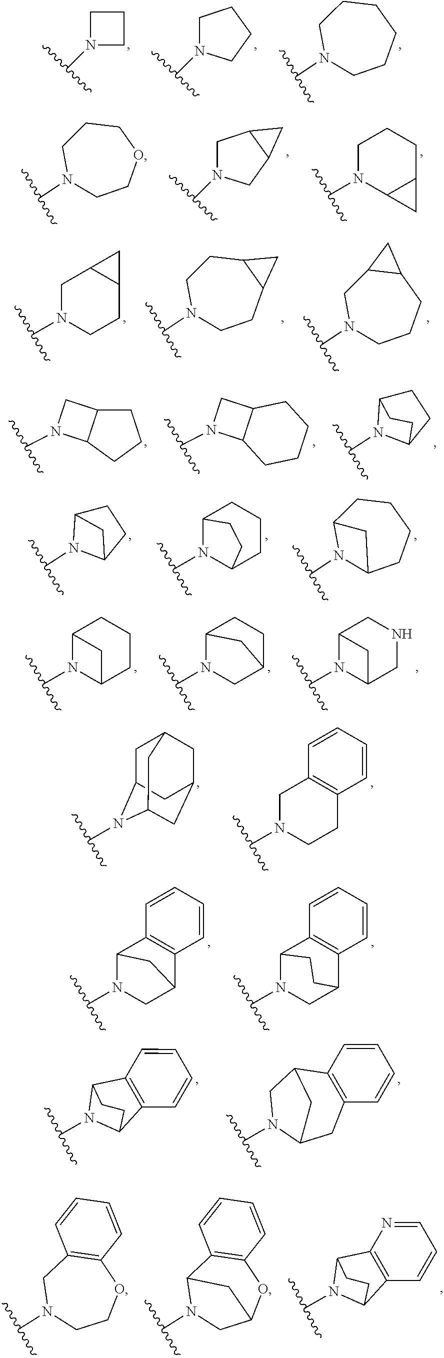

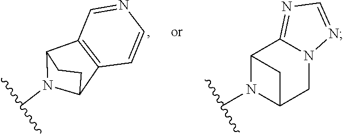 [SMILES: *N1C2CCC1c1cnccc12 ; *N1C2CC1c1ncnn1C2]

wherein each is independently optionally substituted with one to five Z¹.

In certain embodiments, R, the moiety

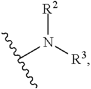

or ring A of the Formulas described herein is:

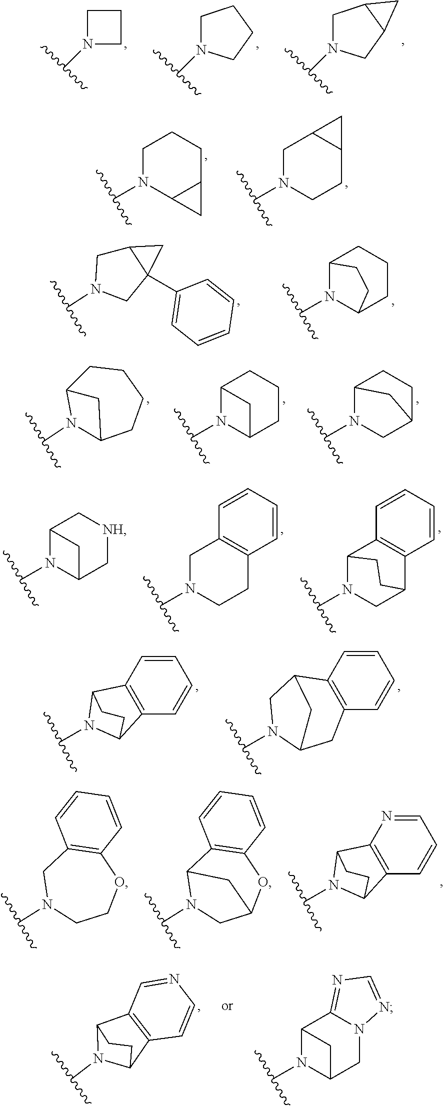

wherein each is independently optionally substituted with one to five Z¹.

In certain embodiments, each Z¹ is independently halo, cyano, C₁₋₆ alkyl, C₁₋₆ haloalkyl, heteroaryl, —OR¹², or —C(O)OR¹²; wherein each C₁₋₆ alkyl, C₁₋₆ haloalkyl, heteroaryl is independently optionally substituted with one to five hydroxy, methoxy, or methyl.

In certain embodiments, each Z¹ is independently halo, cyano, C₁₋₆ alkyl, C₁₋₆haloalkyl, heteroaryl, C₁₋₆ alkoxy, or —C(O)O—C₁₋₆ alkyl; wherein each C₁₋₆ alkyl, C₁₋₆ haloalkyl, heteroaryl is independently optionally substituted with one to five hydroxy, methoxy, or methyl.

In certain embodiments, R, the moiety

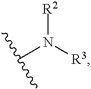

or ring A of the Formulas described herein is:

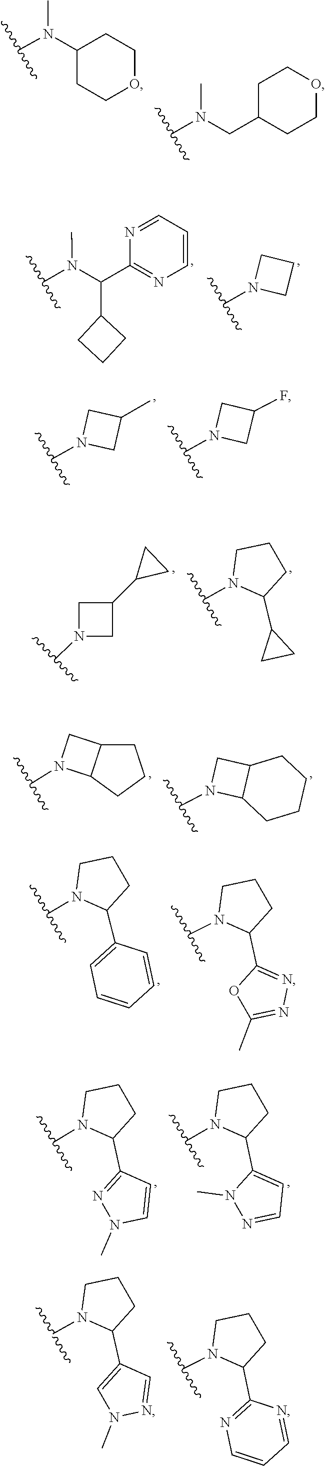

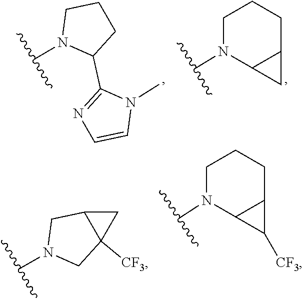

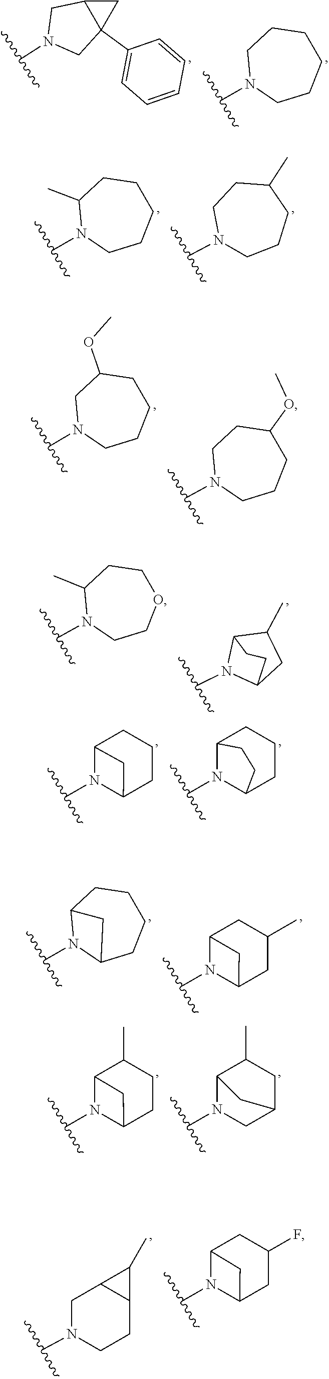

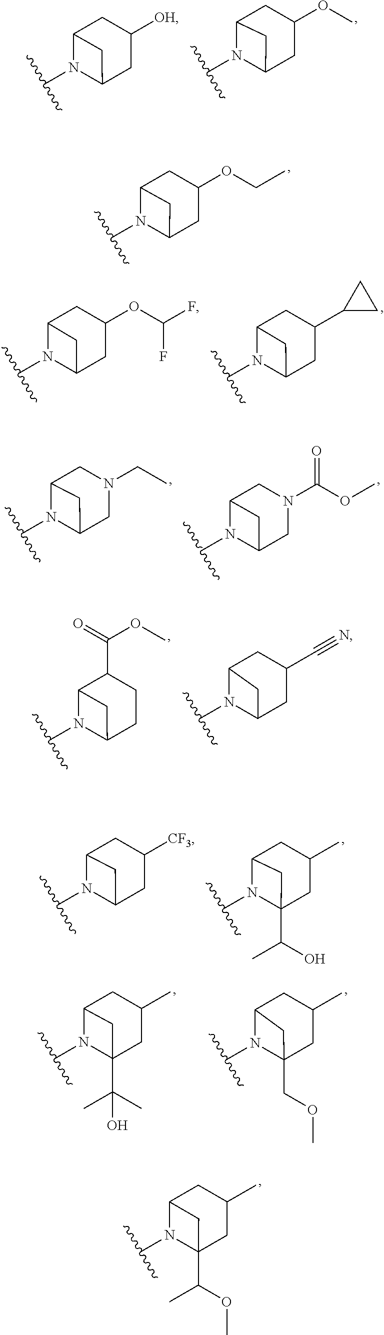

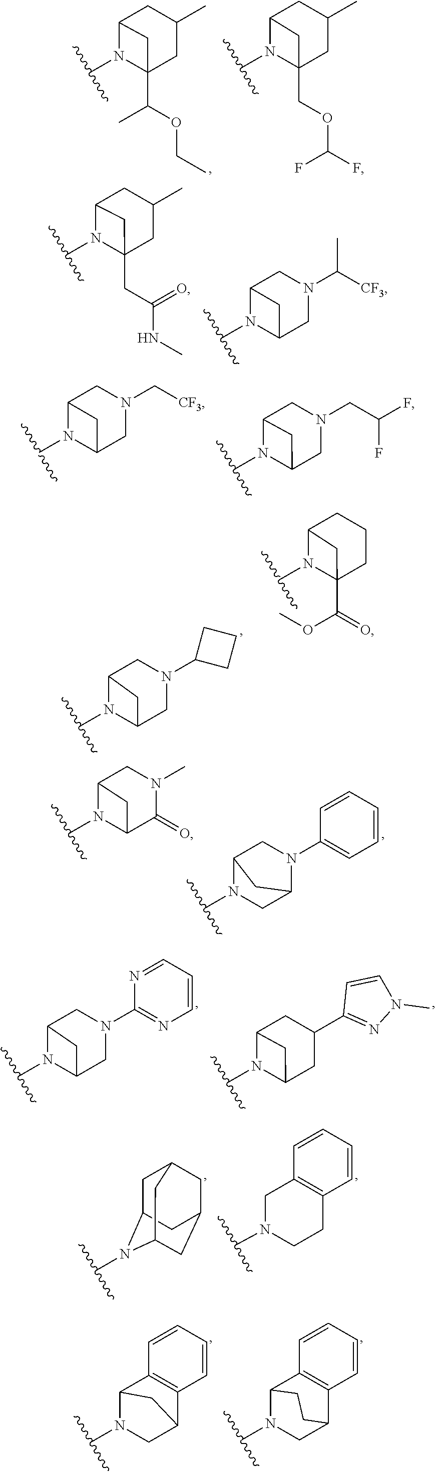

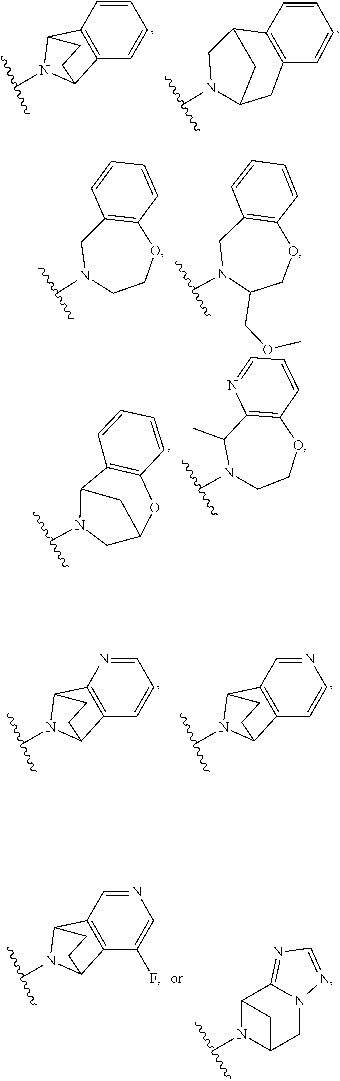

In certain embodiments, R, the moiety

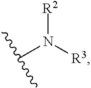

or ring A of the Formulas described herein is:

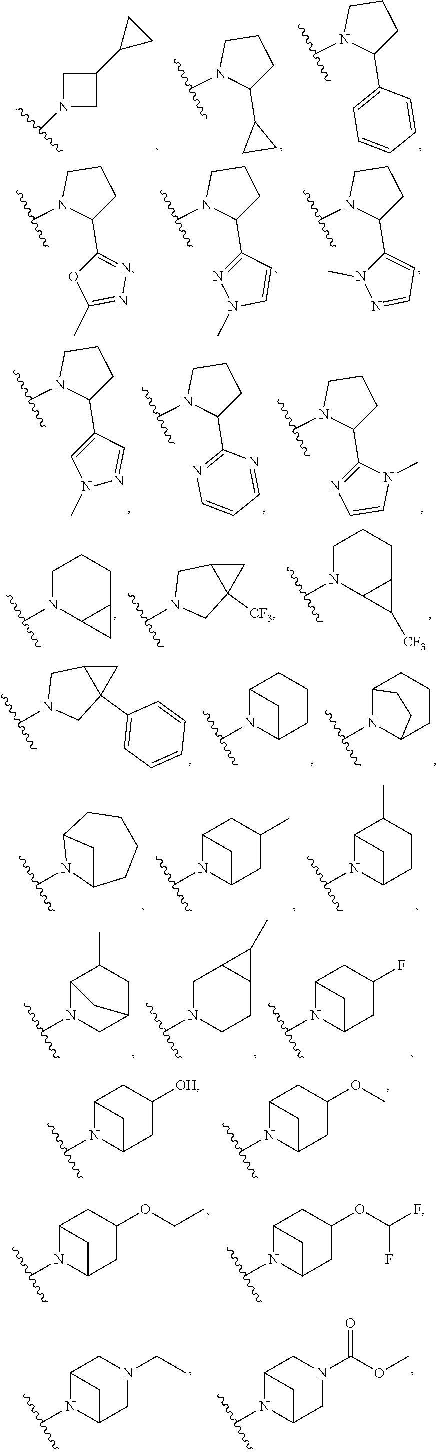

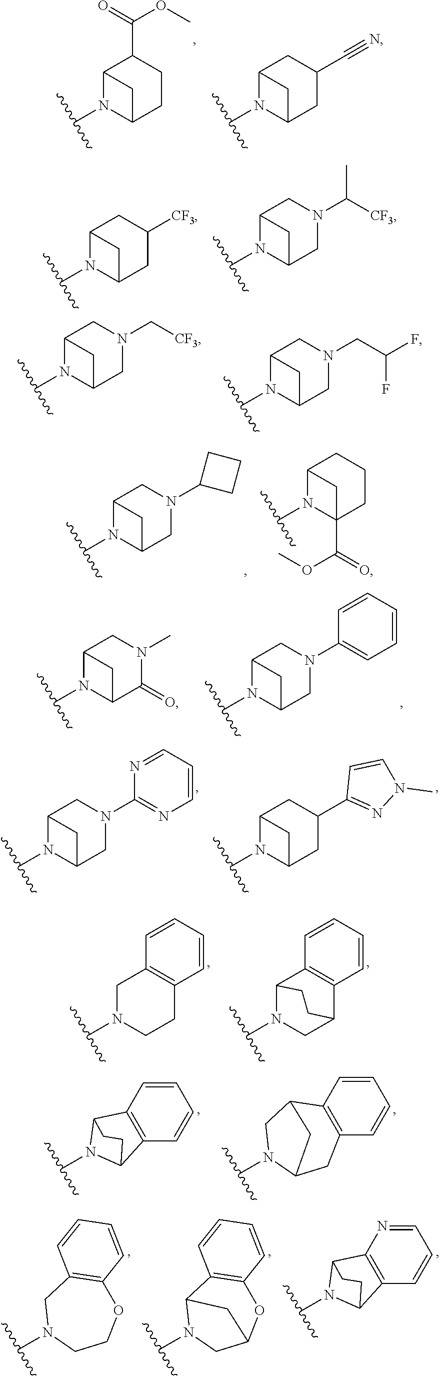

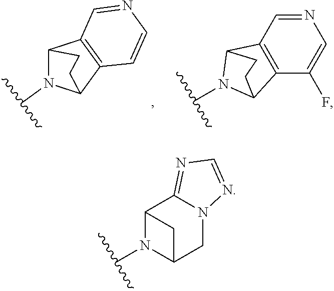 [SMILES: *N1C2CCC1c1cnccc12 ; *N1C2CCC1c1c(F)cncc12 ; *N1C2CC1c1ncnn1C2]

In certain embodiments, R, the moiety

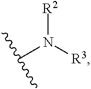

or ring A of the Formulas described herein is:

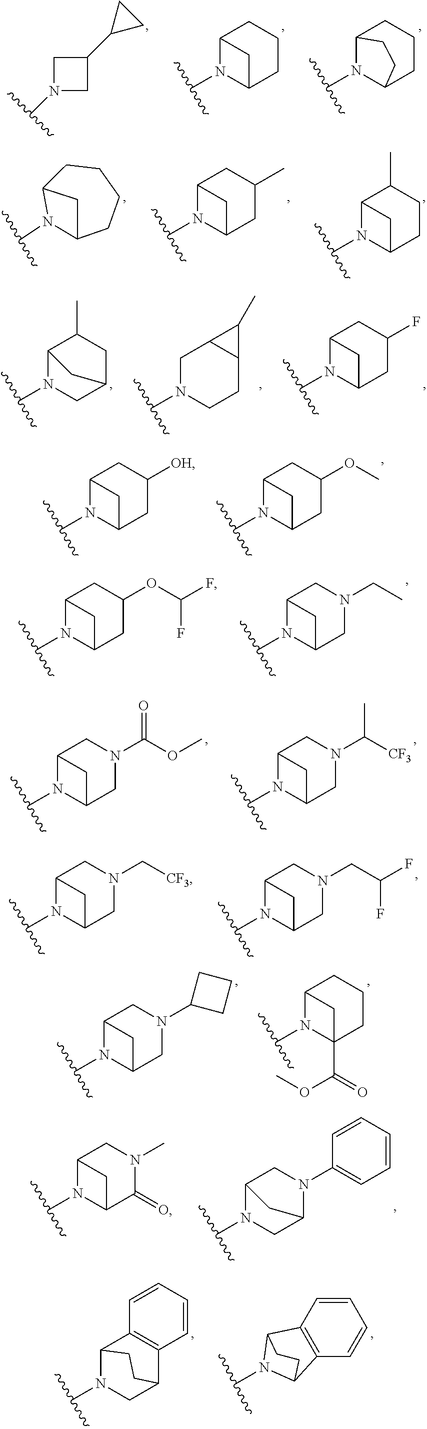

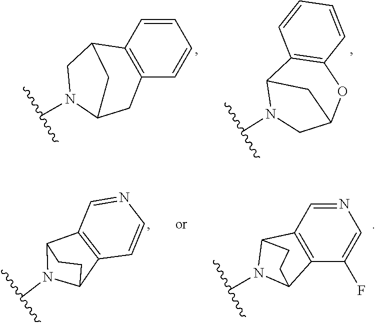 [SMILES: *N1CC2CC1Cc1ccccc12 ; *N1CC2CC1c1ccccc1O2 ; *N1C2CCC1c1cnccc12 ; *N1C2CCC1c1c(F)cncc12]

In certain embodiments, provided is a compound selected from Table 1, or a pharmaceutically acceptable salt, isotopically enriched analog, prodrug, stereoisomer, or a mixture of stereoisomers thereof:

TABLE 1

| Ex. | Structure |
|---|---|
| 1 | 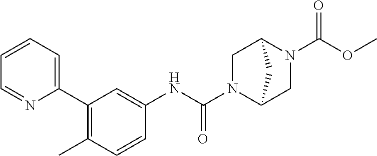 [SMILES: COC(=O)N1C[C@@H]2C[C@H]1CN2C(=O)Nc1ccc(C)c(-c2ccccn2)c1] |
| 2 | 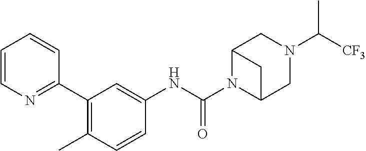 [SMILES: Cc1ccc(NC(=O)N2C3CC2CN(C(C)C(F)(F)F)C3)cc1-c1ccccn1] |
| | mixture of enantiomers |
| 3 | 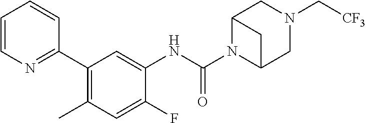 [SMILES: Cc1cc(F)c(NC(=O)N2C3CC2CN(CC(F)(F)F)C3)cc1-c1ccccn1] |
| 4 | 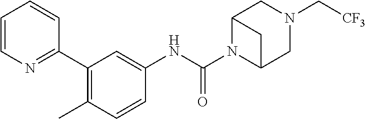 [SMILES: Cc1ccc(NC(=O)N2C3CC2CN(CC(F)(F)F)C3)cc1-c1ccccn1] |
| 5 | 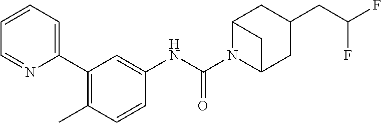 [SMILES: Cc1ccc(NC(=O)N2C3CC(CC(F)F)CC2C3)cc1-c1ccccn1] |
| 6 | 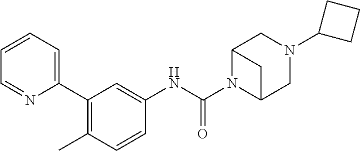 [SMILES: Cc1ccc(NC(=O)N2C3CC2CN(C2CCC2)C3)cc1-c1ccccn1] |
| 7 | 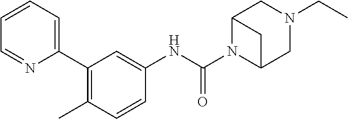 [SMILES: CCN1CC2CC(C1)N2C(=O)Nc1ccc(C)c(-c2ccccn2)c1] |
| 8 | 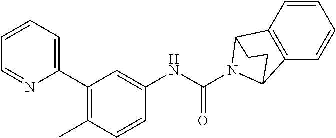 [SMILES: Cc1ccc(NC(=O)N2C3CCC2c2ccccc23)cc1-c1ccccn1] |
| 9 | 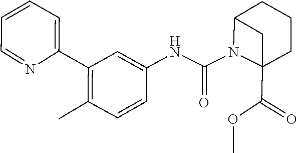 [SMILES: COC(=O)C12CCCC(C1)N2C(=O)Nc1ccc(C)c(-c2ccccn2)c1] |
| | mixture of enantiomers |
| 10 | 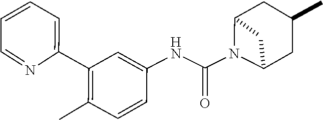 [SMILES: Cc1ccc(NC(=O)N2[C@@H]3C[C@H](C)C[C@H]2C3)cc1-c1ccccn1] |
| 11 | 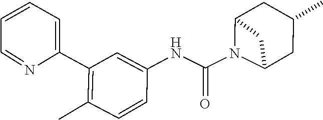 [SMILES: Cc1ccc(NC(=O)N2[C@@H]3C[C@@H](C)C[C@H]2C3)cc1-c1ccccn1] |
| 12 | 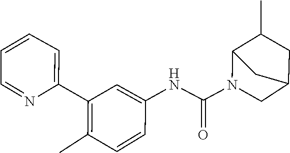 [SMILES: Cc1ccc(NC(=O)N2CC3CC(C)C2C3)cc1-c1ccccn1] |
| | single diastereomer |
| 13 | 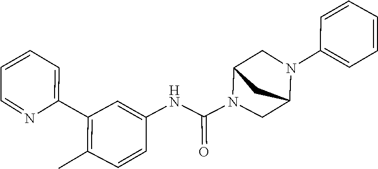 [SMILES: Cc1ccc(NC(=O)N2C[C@@H]3C[C@H]2CN3c2ccccc2)cc1-c1ccccn1] |
| 14 | 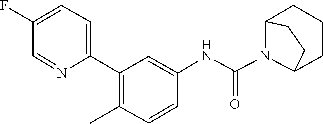 [SMILES: Cc1ccc(NC(=O)N2C3CCCC2CC3)cc1-c1ccc(F)cn1] |
| 15 | 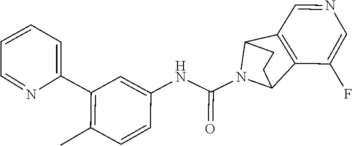 [SMILES: Cc1ccc(NC(=O)N2C3CCC2c2c(F)cncc23)cc1-c1ccccn1] |
| | mixture of enantiomers |
| 16 | 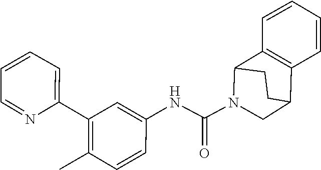 [SMILES: Cc1ccc(NC(=O)N2CC3CCC2c2ccccc23)cc1-c1ccccn1] |
| | mixture of enantiomers |
| 17 | 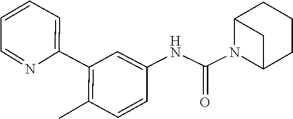 [SMILES: Cc1ccc(NC(=O)N2C3CCCC2C3)cc1-c1ccccn1] |
| 18 | 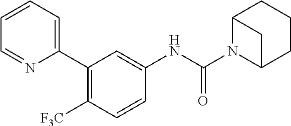 [SMILES: O=C(Nc1ccc(C(F)(F)F)c(-c2ccccn2)c1)N1C2CCCC1C2] |
| 19 | 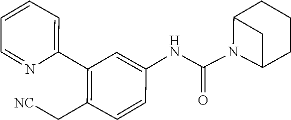 [SMILES: N#CCc1ccc(NC(=O)N2C3CCCC2C3)cc1-c1ccccn1] |
| 20 | 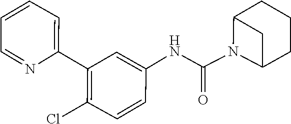 [SMILES: O=C(Nc1ccc(Cl)c(-c2ccccn2)c1)N1C2CCCC1C2] |
| 21 | 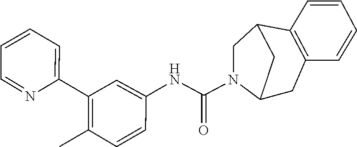 [SMILES: Cc1ccc(NC(=O)N2CC3CC2Cc2ccccc23)cc1-c1ccccn1] |
| | mixture of enantiomers |
| 22 | 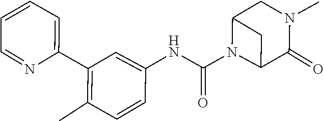 [SMILES: Cc1ccc(NC(=O)N2C3CC2C(=O)N(C)C3)cc1-c1ccccn1] |
| | mixture of enantiomers |
| 23 | 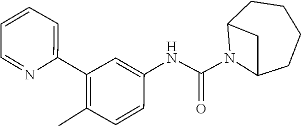 [SMILES: Cc1ccc(NC(=O)N2C3CCCCC2C3)cc1-c1ccccn1] |
| 24 | 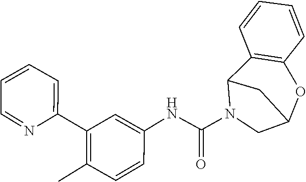 [SMILES: Cc1ccc(NC(=O)N2CC3CC2c2ccccc2O3)cc1-c1ccccn1] |
| | mixture of enantiomers |
| 25 | 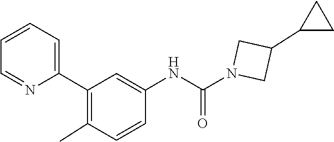 [SMILES: Cc1ccc(NC(=O)N2CC(C3CC3)C2)cc1-c1ccccn1] |
| 26 | 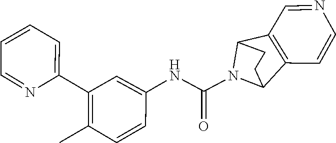 [SMILES: Cc1ccc(NC(=O)N2C3CCC2c2cnccc23)cc1-c1ccccn1] |
| | mixture of enantiomers |
| 27 | 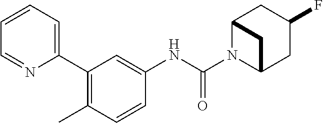 |
| 28 | 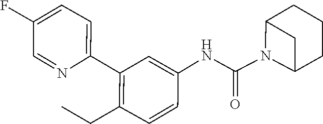 [SMILES: CCc1ccc(NC(=O)N2C3CCCC2C3)cc1-c1ccc(F)cn1] |
| 29 | 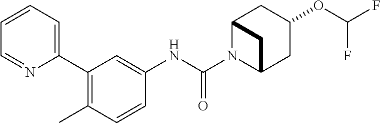 |
| 30 | 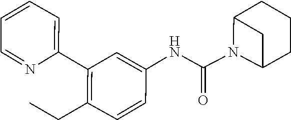 [SMILES: CCc1ccc(NC(=O)N2C3CCCC2C3)cc1-c1ccccn1] |
| 31 | 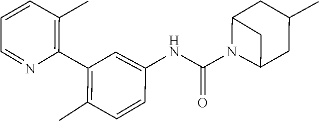 [SMILES: Cc1ccc(NC(=O)N2C3CC(C)CC2C3)cc1-c1ncccc1C] |
| | ~9:1 mixture of cis and trans isomers |
| 32 | 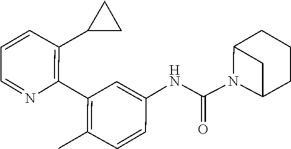 [SMILES: Cc1ccc(NC(=O)N2C3CCCC2C3)cc1-c1ncccc1C1CC1] |
| 33 | 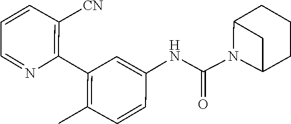 [SMILES: Cc1ccc(NC(=O)N2C3CCCC2C3)cc1-c1ncccc1C#N] |
| 34 | 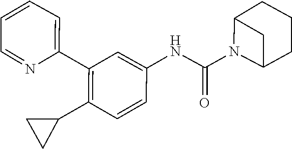 [SMILES: O=C(Nc1ccc(C2CC2)c(-c2ccccn2)c1)N1C2CCCC1C2] |
| 35 | 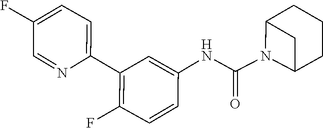 [SMILES: O=C(Nc1ccc(F)c(-c2ccc(F)cn2)c1)N1C2CCCC1C2] |
| 36 | 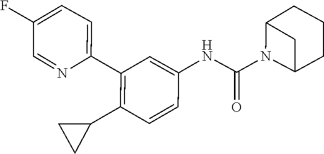 [SMILES: O=C(Nc1ccc(C2CC2)c(-c2ccc(F)cn2)c1)N1C2CCCC1C2] |
| 37 | 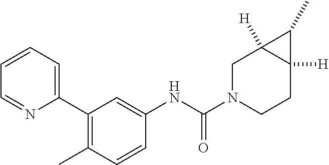 [SMILES: Cc1ccc(NC(=O)N2CC[C@@H]3[C@@H](C)[C@@H]3C2)cc1-c1ccccn1] |
| | mixture of enantiomers |
| 38 | 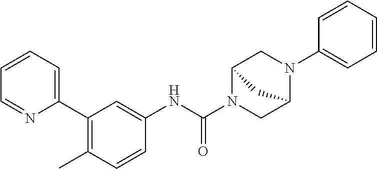 [SMILES: Cc1ccc(NC(=O)N2C[C@H]3C[C@@H]2CN3c2ccccc2)cc1-c1ccccn1] |
| 39 | 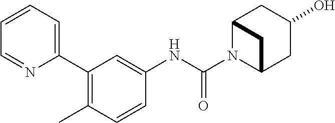 |
| 40 | 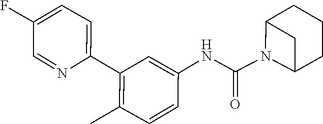 [SMILES: Cc1ccc(NC(=O)N2C3CCCC2C3)cc1-c1ccc(F)cn1] |
| 41 | 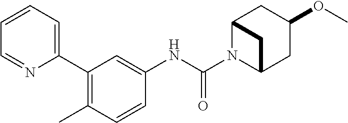 |
| 42 | 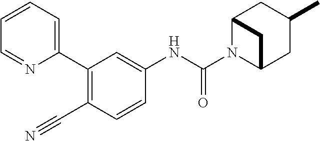 |
| 43 | 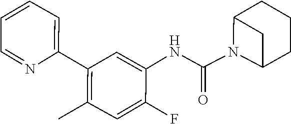 [SMILES: Cc1cc(F)c(NC(=O)N2C3CCCC2C3)cc1-c1ccccn1] |
| 44 | 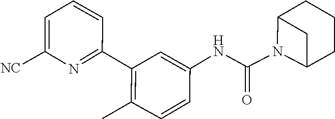 [SMILES: Cc1ccc(NC(=O)N2C3CCCC2C3)cc1-c1cccc(C#N)n1] |
| 45 | 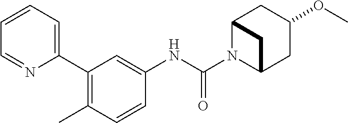 |
| 46 | 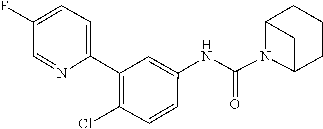 [SMILES: O=C(Nc1ccc(Cl)c(-c2ccc(F)cn2)c1)N1C2CCCC1C2] |
| 47 | 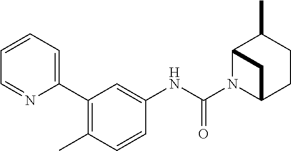 |
| | mixture of cis enantiomers, first eluting peak |
| 48 | 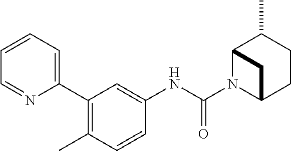 |
| | single trans enantiomer, second eluting peak |
| 49 | 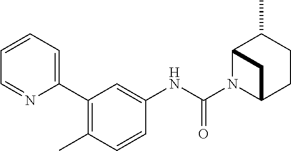 |
| | single trans enantiomer, third eluting peak |
| 50 | 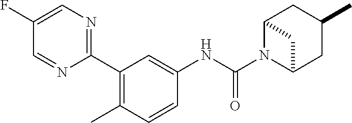 |
| 51 | 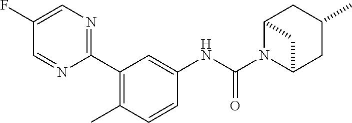 |
| 52 | 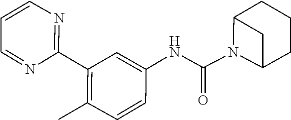 [SMILES: Cc1ccc(NC(=O)N2C3CCCC2C3)cc1-c1ncccn1] |
| 53 | 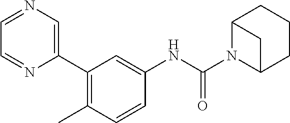 [SMILES: Cc1ccc(NC(=O)N2C3CCCC2C3)cc1-c1cnccn1] |
| 54 | 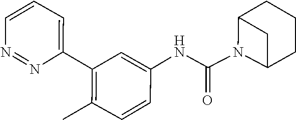 [SMILES: Cc1ccc(NC(=O)N2C3CCCC2C3)cc1-c1cccnn1] |
| 55 | 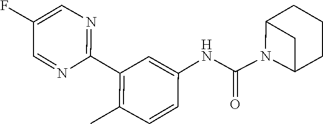 [SMILES: Cc1ccc(NC(=O)N2C3CCCC2C3)cc1-c1ncc(F)cn1] |
| 56 | 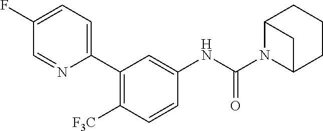 [SMILES: O=C(Nc1ccc(C(F)(F)F)c(-c2ccc(F)cn2)c1)N1C2CCCC1C2] |
| 57 | 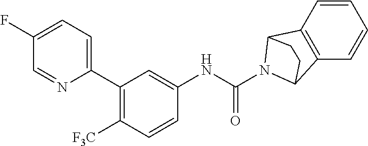 [SMILES: O=C(Nc1ccc(C(F)(F)F)c(-c2ccc(F)cn2)c1)N1C2CCC1c1ccccc12] |
| 58 | 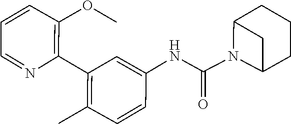 [SMILES: COc1cccnc1-c1cc(NC(=O)N2C3CCCC2C3)ccc1C] |
| 59 | 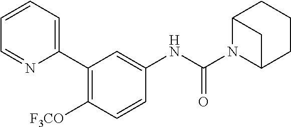 [SMILES: O=C(Nc1ccc(OC(F)(F)F)c(-c2ccccn2)c1)N1C2CCCC1C2] |
| 60 | 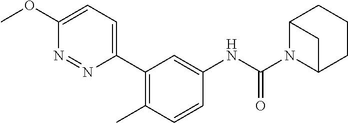 [SMILES: COc1ccc(-c2cc(NC(=O)N3C4CCCC3C4)ccc2C)nn1] |
| 61 | 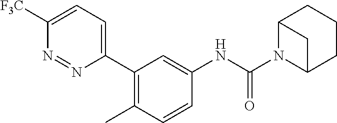 [SMILES: Cc1ccc(NC(=O)N2C3CCCC2C3)cc1-c1ccc(C(F)(F)F)nn1] |
| 62 | 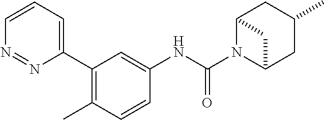 |
| 63 | 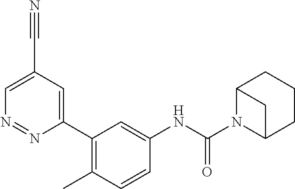 [SMILES: Cc1ccc(NC(=O)N2C3CCCC2C3)cc1-c1cc(C#N)cnn1] |
| 64 | 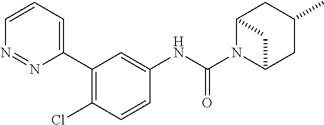 |
| 65 | 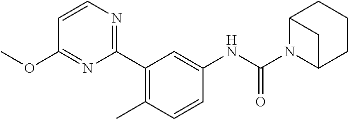 [SMILES: COc1ccnc(-c2cc(NC(=O)N3C4CCCC3C4)ccc2C)n1] |
| 66 | 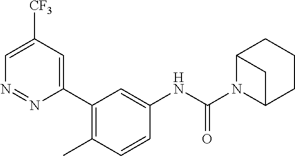 [SMILES: Cc1ccc(NC(=O)N2C3CCCC2C3)cc1-c1cc(C(F)(F)F)cnn1] |
| 67 | 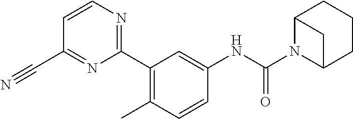 [SMILES: Cc1ccc(NC(=O)N2C3CCCC2C3)cc1-c1nccc(C#N)n1] |
| 68 | 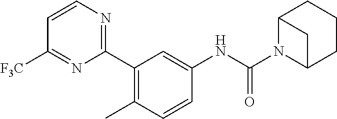 [SMILES: Cc1ccc(NC(=O)N2C3CCCC2C3)cc1-c1nccc(C(F)(F)F)n1] |
| 69 | 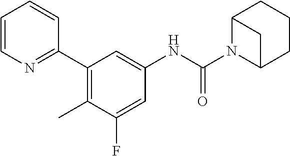 [SMILES: Cc1c(F)cc(NC(=O)N2C3CCCC2C3)cc1-c1ccccn1] |
| 70 | 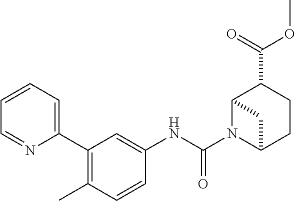 |
| | mixture of enantiomers |
| 71 | 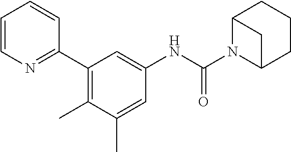 [SMILES: Cc1cc(NC(=O)N2C3CCCC2C3)cc(-c2ccccn2)c1C] |
| 72 | 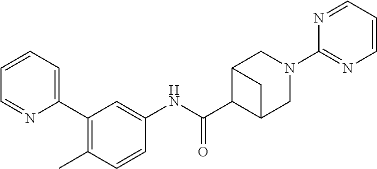 [SMILES: Cc1ccc(NC(=O)C2C3CC2CN(c2ncccn2)C3)cc1-c1ccccn1] |
| 73 | 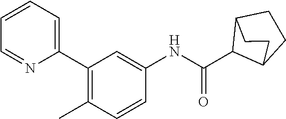 [SMILES: Cc1ccc(NC(=O)C2C3CCC2CC3)cc1-c1ccccn1] |
| 74 | 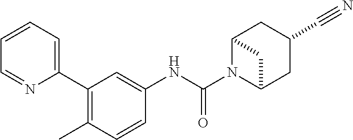 |
| 75 | 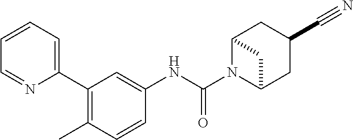 |
| 76 | 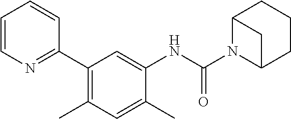 [SMILES: Cc1cc(C)c(-c2ccccn2)cc1NC(=O)N1C2CCCC1C2] |
| 77 | 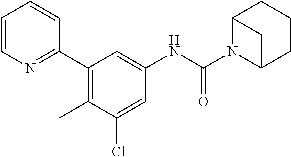 [SMILES: Cc1c(Cl)cc(NC(=O)N2C3CCCC2C3)cc1-c1ccccn1] |
| 78 | 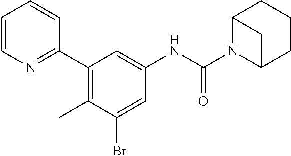 [SMILES: Cc1c(Br)cc(NC(=O)N2C3CCCC2C3)cc1-c1ccccn1] |
| 79 | 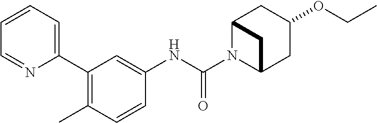 |
| 80 | 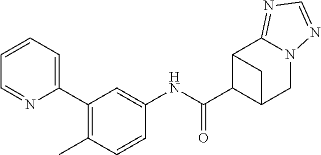 [SMILES: Cc1ccc(NC(=O)C2C3CC2c2ncnn2C3)cc1-c1ccccn1] |
| | mixture of enantiomers |
| 81 | 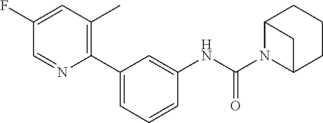 [SMILES: Cc1cc(F)cnc1-c1cccc(NC(=O)N2C3CCCC2C3)c1] |
| 82 | 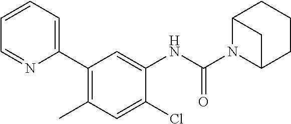 [SMILES: Cc1cc(Cl)c(NC(=O)N2C3CCCC2C3)cc1-c1ccccn1] |
| 83 | 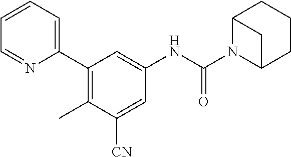 [SMILES: Cc1c(C#N)cc(NC(=O)N2C3CCCC2C3)cc1-c1ccccn1] |
| 84 | 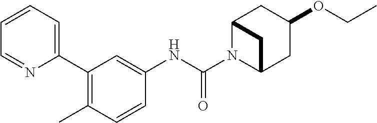 |
| 85 | 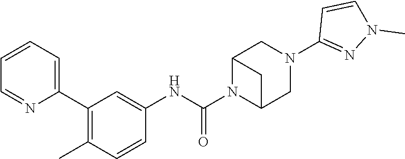 [SMILES: Cc1ccc(NC(=O)N2C3CC2CN(c2ccn(C)n2)C3)cc1-c1ccccn1] |
| 86 | 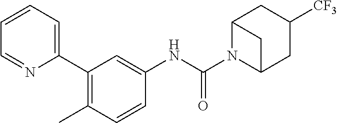 [SMILES: Cc1ccc(NC(=O)N2C3CC2CC(C(F)(F)F)C3)cc1-c1ccccn1] |
| | Single unknown diastereomer |
| 87 | 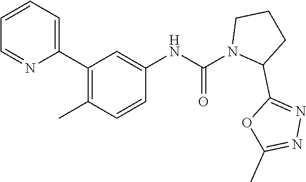 [SMILES: Cc1nnc(C2CCCN2C(=O)Nc2ccc(C)c(-c3ccccn3)c2)o1] |
| 88 | 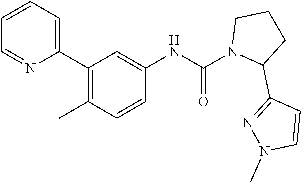 [SMILES: Cc1ccc(NC(=O)N2CCCC2c2ccn(C)n2)cc1-c1ccccn1] |
| | mixture of enantiomers |
| 89 | 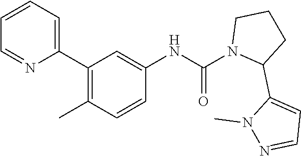 [SMILES: Cc1ccc(NC(=O)N2CCCC2c2ccnn2C)cc1-c1ccccn1] |
| | mixture of enantiomers |
| 90 | 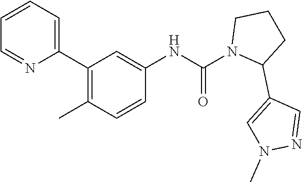 [SMILES: Cc1ccc(NC(=O)N2CCCC2c2cnn(C)c2)cc1-c1ccccn1] |
| | mixture of enantiomers |
| 91 | 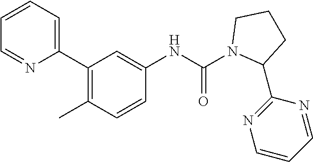 [SMILES: Cc1ccc(NC(=O)N2CCCC2c2ncccn2)cc1-c1ccccn1] |
| | mixture of enantiomers |
| 92 | 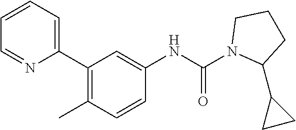 [SMILES: Cc1ccc(NC(=O)N2CCCC2C2CC2)cc1-c1ccccn1] |
| | mixture of enantiomers |
| 93 | 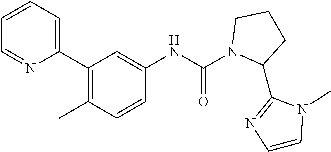 [SMILES: Cc1ccc(NC(=O)N2CCCC2c2nccn2C)cc1-c1ccccn1] |
| | mixture of enantiomers |
| 94 | 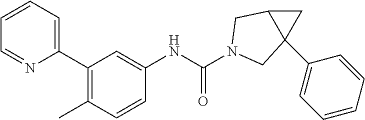 [SMILES: Cc1ccc(NC(=O)N2CC3CC3(c3ccccc3)C2)cc1-c1ccccn1] |
| | mixture of enantiomers |
| 95 | 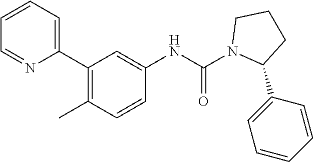 [SMILES: Cc1ccc(NC(=O)N2CCC[C@@H]2c2ccccc2)cc1-c1ccccn1] |
| 96 | 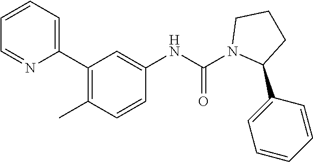 [SMILES: Cc1ccc(NC(=O)N2CCC[C@H]2c2ccccc2)cc1-c1ccccn1] |
| 97 | 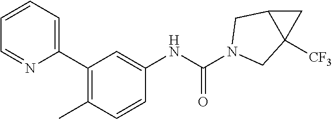 [SMILES: Cc1ccc(NC(=O)N2CC3CC3(C(F)(F)F)C2)cc1-c1ccccn1] |
| | mixture of enantiomers |
| 98 | 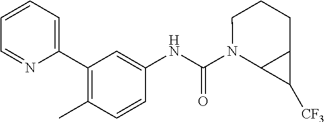 [SMILES: Cc1ccc(NC(=O)N2CCCC3C2C3C(F)(F)F)cc1-c1ccccn1] |
| | mixture of enantiomers |
| 99 | 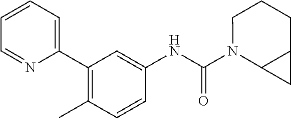 [SMILES: Cc1ccc(NC(=O)N2CCCC3CC32)cc1-c1ccccn1] |
| | mixture of enantiomers |
| 100 | 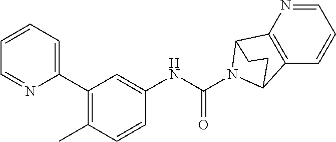 [SMILES: Cc1ccc(NC(=O)N2C3CCC2c2ncccc23)cc1-c1ccccn1] |
| | mixture of enantiomers |
| 101 | 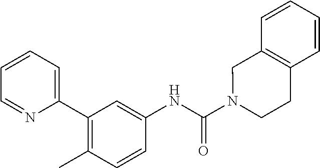 [SMILES: Cc1ccc(NC(=O)N2CCc3ccccc3C2)cc1-c1ccccn1] |
| 102 | 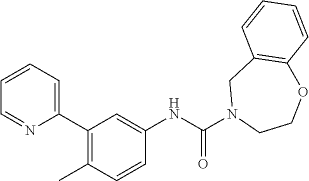 [SMILES: Cc1ccc(NC(=O)N2CCOc3ccccc3C2)cc1-c1ccccn1] |
| 103 | 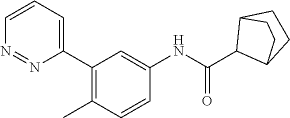 [SMILES: Cc1ccc(NC(=O)C2C3CCC2CC3)cc1-c1cccnn1] |
| 104 | 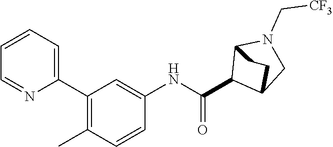 [SMILES: Cc1ccc(NC(=O)[C@@H]2[C@@H]3CC[C@H]2N(CC(F)(F)F)C3)cc1-c1ccccn1] |
| | trans-, single unknown enantiomer, second |
| | eluting peak |
| 105 | 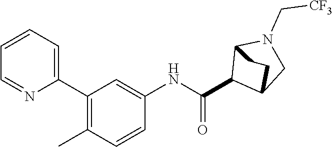 [SMILES: Cc1ccc(NC(=O)[C@@H]2[C@@H]3CC[C@H]2N(CC(F)(F)F)C3)cc1-c1ccccn1] |
| | trans-, single unknown enantiomer, first eluting |
| | peak |
| 106 | 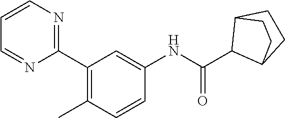 [SMILES: Cc1ccc(NC(=O)C2C3CCC2CC3)cc1-c1ncccn1] |
| 107 | 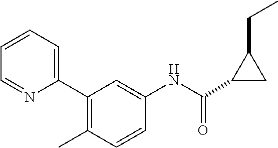 [SMILES: CC[C@H]1C[C@@H]1C(=O)Nc1ccc(C)c(-c2ccccn2)c1] |
| | trans-, single unknown enantiomer |
| 108 | 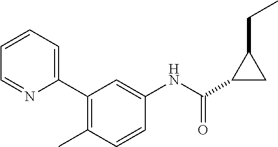 [SMILES: CC[C@H]1C[C@@H]1C(=O)Nc1ccc(C)c(-c2ccccn2)c1] |
| | trans-, single unknown enantiomer |
| 109 | 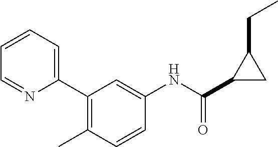 [SMILES: CC[C@H]1C[C@H]1C(=O)Nc1ccc(C)c(-c2ccccn2)c1] |
| | cis-, single unknown enantiomer |
| 110 | 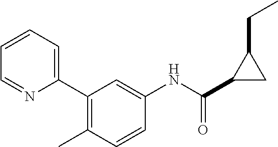 [SMILES: CC[C@H]1C[C@H]1C(=O)Nc1ccc(C)c(-c2ccccn2)c1] |
| | cis-, single unknown enantiomer |
| 111 | 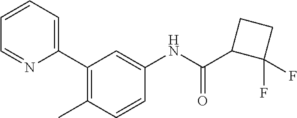 [SMILES: Cc1ccc(NC(=O)C2CCC2(F)F)cc1-c1ccccn1] |
| | mixture of enantiomers |
| 112 | 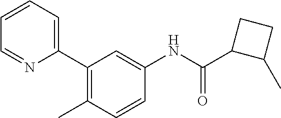 [SMILES: Cc1ccc(NC(=O)C2CCC2C)cc1-c1ccccn1] |
| | mixture of enantiomers |
| 113 | 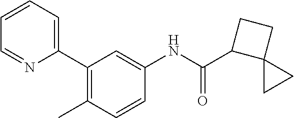 [SMILES: Cc1ccc(NC(=O)C2CCC23CC3)cc1-c1ccccn1] |
| | mixture of enantiomers |
| 114 | 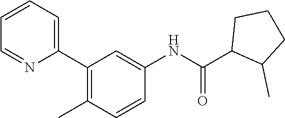 [SMILES: Cc1ccc(NC(=O)C2CCCC2C)cc1-c1ccccn1] |
| | mixture of enantiomers |
| 115 | 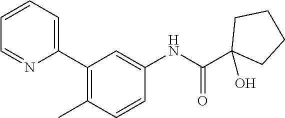 [SMILES: Cc1ccc(NC(=O)C2(O)CCCC2)cc1-c1ccccn1] |
| 116 | 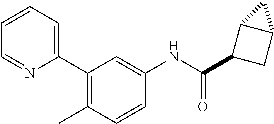 |
| | mixture of enantiomers |
| 117 | 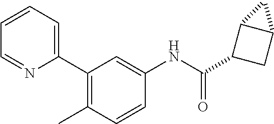 |
| | mixture of enantiomers |
| 118 | 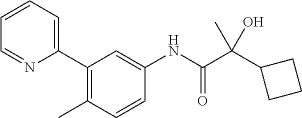 [SMILES: Cc1ccc(NC(=O)C(C)(O)C2CCC2)cc1-c1ccccn1] |
| | mixture of enantiomers |
| 119 | 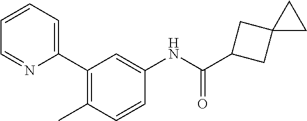 [SMILES: Cc1ccc(NC(=O)C2CC3(CC3)C2)cc1-c1ccccn1] |
| 120 | 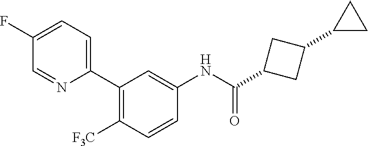 [SMILES: O=C(Nc1ccc(C(F)(F)F)c(-c2ccc(F)cn2)c1)[C@H]1C[C@@H](C2CC2)C1] |
| 121 | 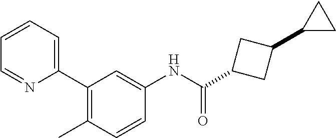 [SMILES: Cc1ccc(NC(=O)[C@H]2C[C@H](C3CC3)C2)cc1-c1ccccn1] |
| 122 | 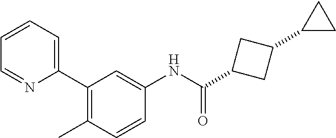 [SMILES: Cc1ccc(NC(=O)[C@H]2C[C@@H](C3CC3)C2)cc1-c1ccccn1] |
| 123 | 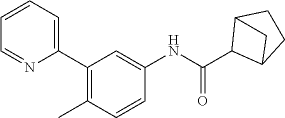 [SMILES: Cc1ccc(NC(=O)C2C3CCC2C3)cc1-c1ccccn1] |
| 124 | 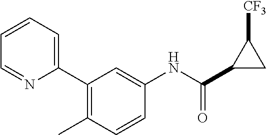 [SMILES: Cc1ccc(NC(=O)[C@@H]2C[C@@H]2C(F)(F)F)cc1-c1ccccn1] |
| | mixture of enantiomers |
| 125 | 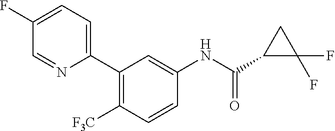 [SMILES: O=C(Nc1ccc(C(F)(F)F)c(-c2ccc(F)cn2)c1)[C@@H]1CC1(F)F] |
| 126 | 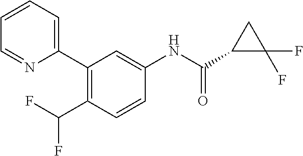 [SMILES: O=C(Nc1ccc(C(F)F)c(-c2ccccn2)c1)[C@@H]1CC1(F)F] |
| 127 | 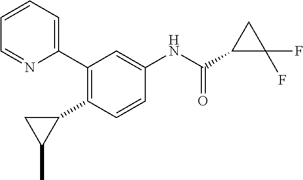 |
| | mixture of diastereomers |
| 128 | 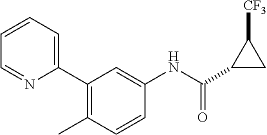 [SMILES: Cc1ccc(NC(=O)[C@H]2C[C@@H]2C(F)(F)F)cc1-c1ccccn1] |
| | mixture of enantiomers |
| 129 | 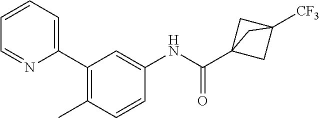 [SMILES: Cc1ccc(NC(=O)C23CC(C(F)(F)F)(C2)C3)cc1-c1ccccn1] |
| 130 | 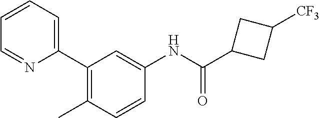 [SMILES: Cc1ccc(NC(=O)C2CC(C(F)(F)F)C2)cc1-c1ccccn1] |
| | mixture of diastereomers |
| 131 | 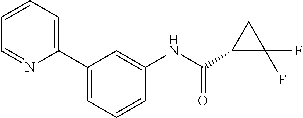 [SMILES: O=C(Nc1cccc(-c2ccccn2)c1)[C@@H]1CC1(F)F] |
| 132 | 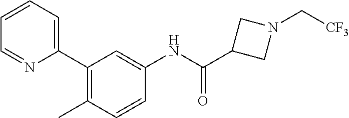 [SMILES: Cc1ccc(NC(=O)C2CN(CC(F)(F)F)C2)cc1-c1ccccn1] |
| 133 | 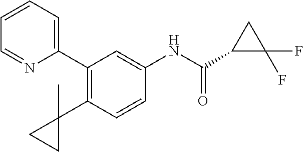 [SMILES: CC1(c2ccc(NC(=O)[C@@H]3CC3(F)F)cc2-c2ccccn2)CC1] |
| 134 | 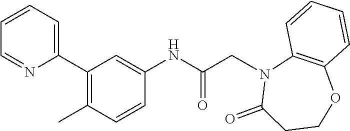 [SMILES: Cc1ccc(NC(=O)CN2C(=O)CCOc3ccccc32)cc1-c1ccccn1] |
| 135 | 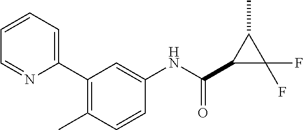 [SMILES: Cc1ccc(NC(=O)[C@H]2[C@H](C)C2(F)F)cc1-c1ccccn1] |
| | mixture of enantiomers |
| 136 | 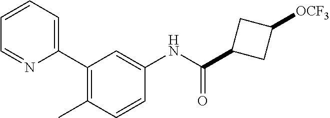 |
| 137 | 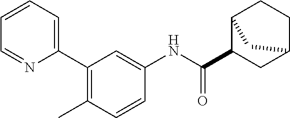 |
| | trans-, single unknown enantiomer, fourth eluting |
| | peak |
| 138 | 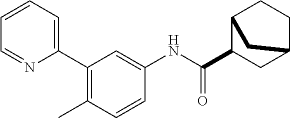 |
| | cis-, single unknown enantiomer, second eluting |
| | peak |
| 139 | 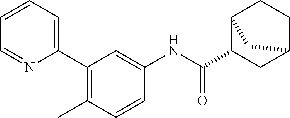 |
| | cis-, single unknown enantiomer, third eluting |
| | peak |
| 140 | 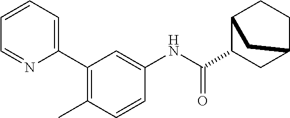 |
| | trans-, single unknown enantiomer, first eluting |
| | peak |
| 141 | 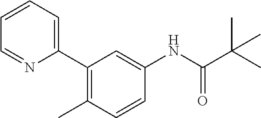 [SMILES: Cc1ccc(NC(=O)C(C)(C)C)cc1-c1ccccn1] |
| 142 | 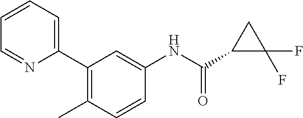 [SMILES: Cc1ccc(NC(=O)[C@@H]2CC2(F)F)cc1-c1ccccn1] |
| 143 | 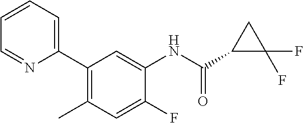 [SMILES: Cc1cc(F)c(NC(=O)[C@@H]2CC2(F)F)cc1-c1ccccn1] |
| 144 | 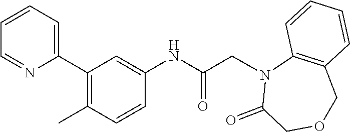 [SMILES: Cc1ccc(NC(=O)CN2C(=O)COCc3ccccc32)cc1-c1ccccn1] |
| 145 | 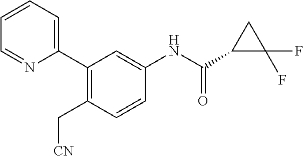 [SMILES: N#CCc1ccc(NC(=O)[C@@H]2CC2(F)F)cc1-c1ccccn1] |
| 146 | 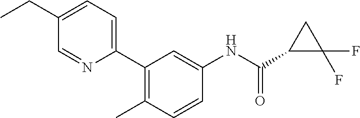 [SMILES: CCc1ccc(-c2cc(NC(=O)[C@@H]3CC3(F)F)ccc2C)nc1] |
| 147 | 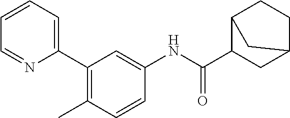 [SMILES: Cc1ccc(NC(=O)C2CC3CCC2C3)cc1-c1ccccn1] |
| | mixture of diastereomers |
| 148 | 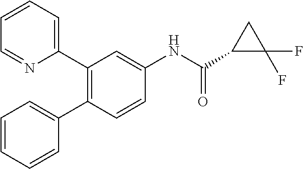 [SMILES: O=C(Nc1ccc(-c2ccccc2)c(-c2ccccn2)c1)[C@@H]1CC1(F)F] |
| 149 | 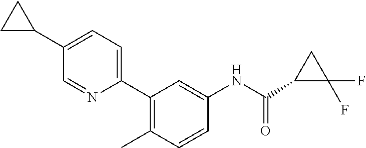 [SMILES: Cc1ccc(NC(=O)[C@@H]2CC2(F)F)cc1-c1ccc(C2CC2)cn1] |
| 150 | 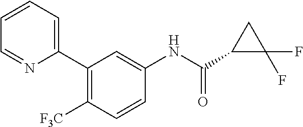 [SMILES: O=C(Nc1ccc(C(F)(F)F)c(-c2ccccn2)c1)[C@@H]1CC1(F)F] |
| 151 | 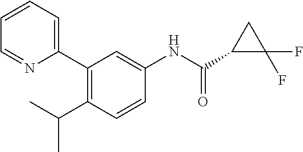 [SMILES: CC(C)c1ccc(NC(=O)[C@@H]2CC2(F)F)cc1-c1ccccn1] |
| 152 | 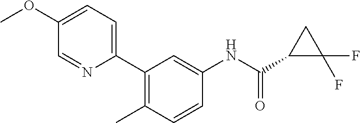 [SMILES: COc1ccc(-c2cc(NC(=O)[C@@H]3CC3(F)F)ccc2C)nc1] |
| 153 | 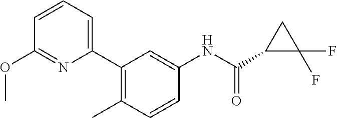 [SMILES: COc1cccc(-c2cc(NC(=O)[C@@H]3CC3(F)F)ccc2C)n1] |
| 154 | 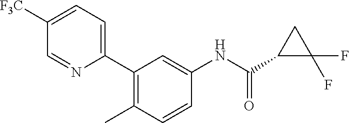 [SMILES: Cc1ccc(NC(=O)[C@@H]2CC2(F)F)cc1-c1ccc(C(F)(F)F)cn1] |
| 155 | 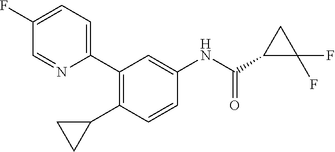 [SMILES: O=C(Nc1ccc(C2CC2)c(-c2ccc(F)cn2)c1)[C@@H]1CC1(F)F] |
| 156 | 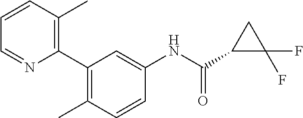 [SMILES: Cc1ccc(NC(=O)[C@@H]2CC2(F)F)cc1-c1ncccc1C] |
| 157 | 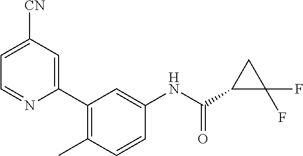 [SMILES: Cc1ccc(NC(=O)[C@@H]2CC2(F)F)cc1-c1cc(C#N)ccn1] |
| 158 | 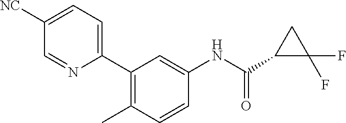 [SMILES: Cc1ccc(NC(=O)[C@@H]2CC2(F)F)cc1-c1ccc(C#N)cn1] |
| 159 | 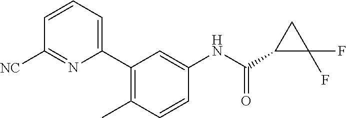 [SMILES: Cc1ccc(NC(=O)[C@@H]2CC2(F)F)cc1-c1cccc(C#N)n1] |
| 160 | 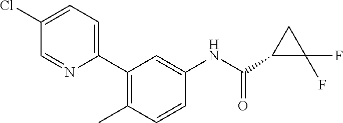 [SMILES: Cc1ccc(NC(=O)[C@@H]2CC2(F)F)cc1-c1ccc(Cl)cn1] |
| 161 | 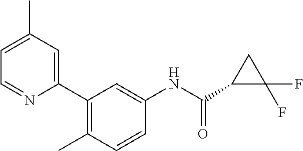 [SMILES: Cc1ccnc(-c2cc(NC(=O)[C@@H]3CC3(F)F)ccc2C)c1] |
| 162 | 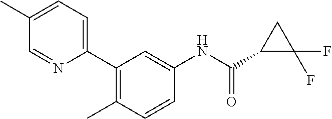 [SMILES: Cc1ccc(-c2cc(NC(=O)[C@@H]3CC3(F)F)ccc2C)nc1] |
| 163 | 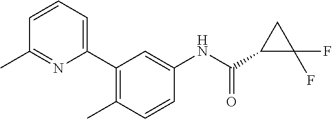 [SMILES: Cc1cccc(-c2cc(NC(=O)[C@@H]3CC3(F)F)ccc2C)n1] |
| 164 | 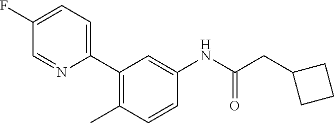 [SMILES: Cc1ccc(NC(=O)CC2CCC2)cc1-c1ccc(F)cn1] |
| 165 | 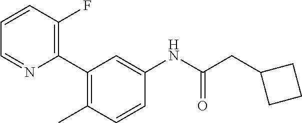 [SMILES: Cc1ccc(NC(=O)CC2CCC2)cc1-c1ncccc1F] |
| 166 | 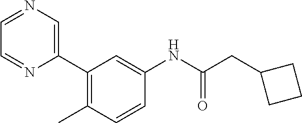 [SMILES: Cc1ccc(NC(=O)CC2CCC2)cc1-c1cnccn1] |
| 167 | 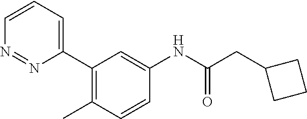 [SMILES: Cc1ccc(NC(=O)CC2CCC2)cc1-c1cccnn1] |
| 168 | 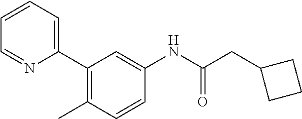 [SMILES: Cc1ccc(NC(=O)CC2CCC2)cc1-c1ccccn1] |
| 169 | 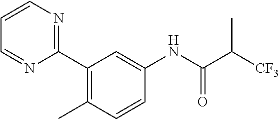 [SMILES: Cc1ccc(NC(=O)C(C)C(F)(F)F)cc1-c1ncccn1] |
| | mixture of enantiomers |
| 170 | 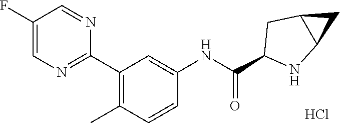 [SMILES: Cc1ccc(NC(=O)[C@H]2C[C@H]3C[C@H]3N2)cc1-c1ncc(F)cn1 ; Cl] |
| 171 | 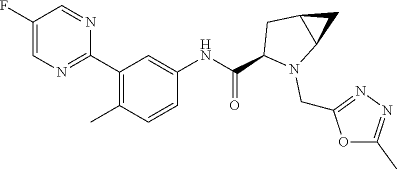 [SMILES: Cc1nnc(CN2[C@@H](C(=O)Nc3ccc(C)c(-c4ncc(F)cn4)c3)C[C@H]3C[C@H]32)o1] |
| 172 | 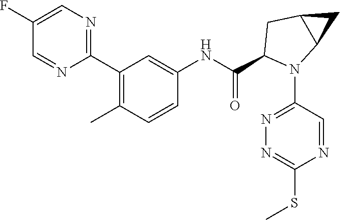 [SMILES: CSc1ncc(N2[C@@H](C(=O)Nc3ccc(C)c(-c4ncc(F)cn4)c3)C[C@H]3C[C@H]32)nn1] |
| 173 | 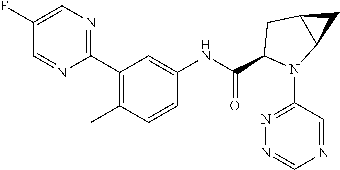 [SMILES: Cc1ccc(NC(=O)[C@H]2C[C@H]3C[C@H]3N2c2cncnn2)cc1-c1ncc(F)cn1] |
| 174 | 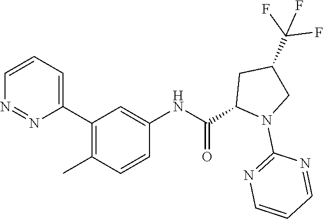 [SMILES: Cc1ccc(NC(=O)[C@@H]2C[C@H](C(F)(F)F)CN2c2ncccn2)cc1-c1cccnn1] |
| 175 | 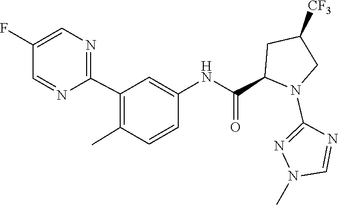 [SMILES: Cc1ccc(NC(=O)[C@H]2C[C@@H](C(F)(F)F)CN2c2ncn(C)n2)cc1-c1ncc(F)cn1] |
| 176 | 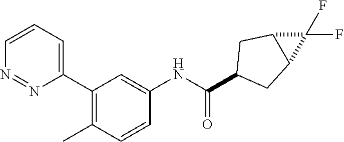 [SMILES: Cc1ccc(NC(=O)[C@@H]2C[C@@H]3[C@H](C2)C3(F)F)cc1-c1cccnn1] |
| 177 | 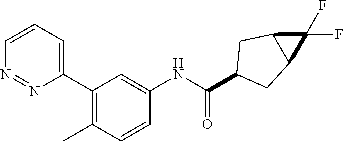 [SMILES: Cc1ccc(NC(=O)[C@H]2C[C@@H]3[C@H](C2)C3(F)F)cc1-c1cccnn1] |
| 178 | 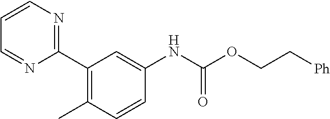 [SMILES: Cc1ccc(NC(=O)OCCc2ccccc2)cc1-c1ncccn1] |
| 179 | 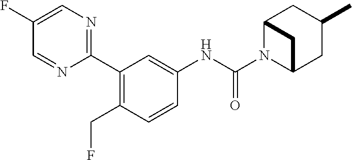 |
| 180 | 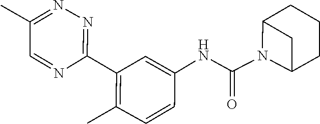 [SMILES: Cc1cnc(-c2cc(NC(=O)N3C4CCCC3C4)ccc2C)nn1] |
| 181 | 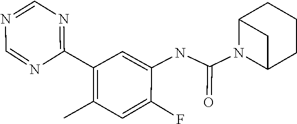 |
| 182 | 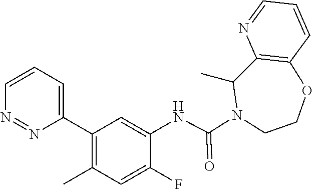 [SMILES: Cc1cc(F)c(NC(=O)N2CCOc3cccnc3C2C)cc1-c1cccnn1] |
| 183 | 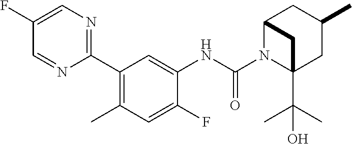 |
| | mixture of enantiomers |
| 184 | 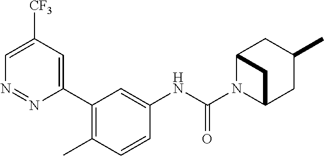 |
| 185 | 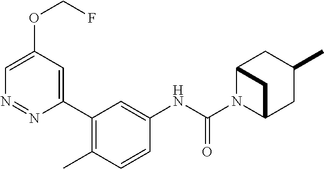 |
| 186 | 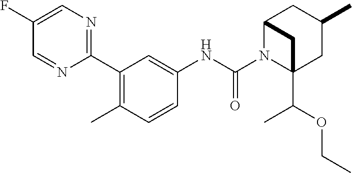 |
| | single unknown enantiomer, first eluting |
| 187 | 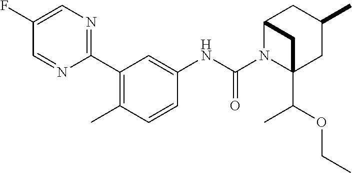 |
| | single unknown enantiomer, second eluting |
| 188 | 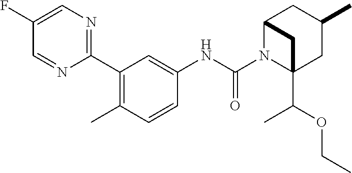 |
| | single unknown enantiomer, first eluting |
| 189 | 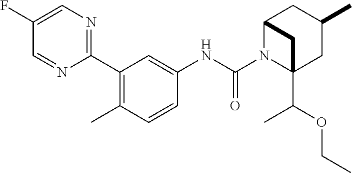 |
| | single unknown enantiomer, second eluting |
| 190 | 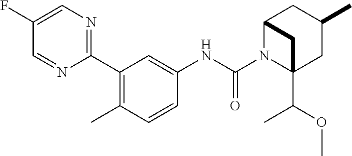 |
| | mixture of enantiomers |
| 191 | 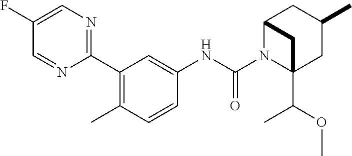 |
| | single unknown enantiomer, first eluting peak |
| 192 | 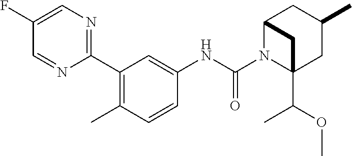 |
| | single unknown enantiomer, second eluting peak |
| 193 | 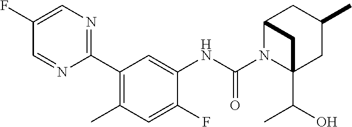 |
| | single unknown enantiomer, first eluting peak |
| 194 | 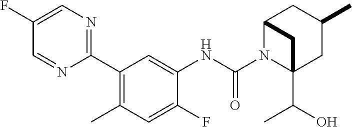 |
| | single unknown enantiomer, second eluting peak |
| 195 | 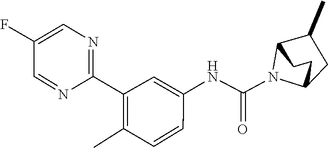 |
| | single unknown enantiomer, first eluting peak |
| 196 | 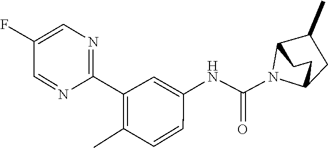 |
| | single unknown enantiomer, second eluting peak |
| 197 | 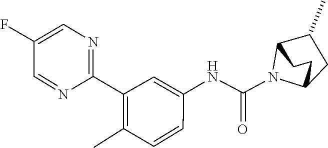 |
| | single unknown enantiomer, first eluting peak |
| 198 | 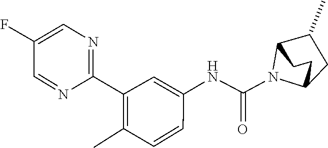 |
| | single unknown enantiomer, second eluting peak |
| 199 | 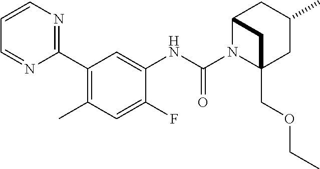 |
| | single unknown enantiomer, first eluting peak |
| 200 | 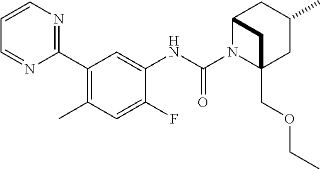 |
| | single unknown enantiomer, second eluting peak |
| 201 | 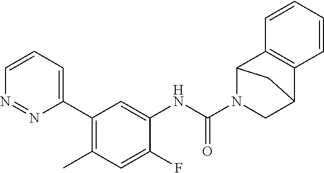 [SMILES: Cc1cc(F)c(NC(=O)N2CC3CC2c2ccccc23)cc1-c1cccnn1] |
| | single unknown enantiomer, first eluting peak |
| 202 | 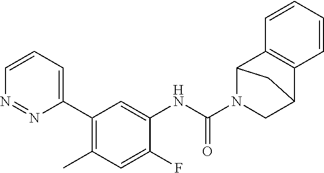 [SMILES: Cc1cc(F)c(NC(=O)N2CC3CC2c2ccccc23)cc1-c1cccnn1] |
| | single unknown enantiomer, second eluting peak |
| 203 | 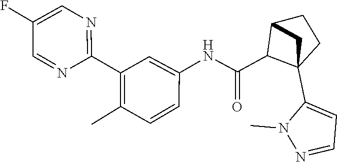 [SMILES: Cc1ccc(NC(=O)C2[C@@H]3CC[C@@]2(c2ccnn2C)C3)cc1-c1ncc(F)cn1] |
| | single unknown enantiomer, first eluting peak |
| 204 | 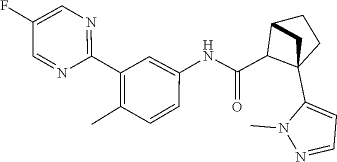 [SMILES: Cc1ccc(NC(=O)C2[C@@H]3CC[C@@]2(c2ccnn2C)C3)cc1-c1ncc(F)cn1] |
| | single unknown enantiomer, second eluting peak |
| 205 | 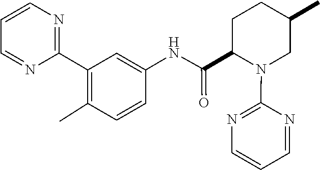 [SMILES: Cc1ccc(NC(=O)[C@H]2CC[C@@H](C)CN2c2ncccn2)cc1-c1ncccn1] |
| | single unknown enantiomer, first eluting peak |
| 206 | 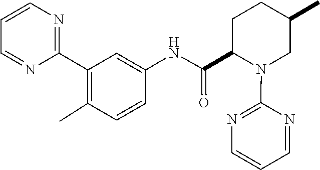 [SMILES: Cc1ccc(NC(=O)[C@H]2CC[C@@H](C)CN2c2ncccn2)cc1-c1ncccn1] |
| | single unknown enantiomer, second eluting peak |
| 207 | 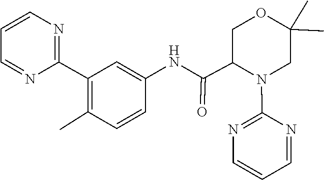 [SMILES: Cc1ccc(NC(=O)C2COC(C)(C)CN2c2ncccn2)cc1-c1ncccn1] |
| | single unknown enantiomer, first eluting peak |
| 208 | 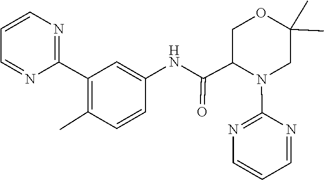 [SMILES: Cc1ccc(NC(=O)C2COC(C)(C)CN2c2ncccn2)cc1-c1ncccn1] |
| | single unknown enantiomer, second eluting peak |
| 209 | 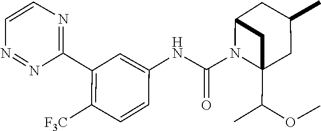 |
| | single unknown enantiomer, first eluting peak |
| 210 | 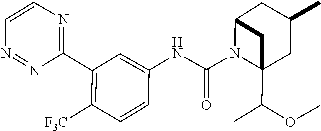 |
| | single unknown enantiomer, second eluting peak |
| 211 | 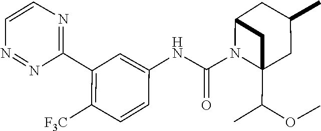 |
| | single unknown enantiomer, first eluting peak |
| 212 | 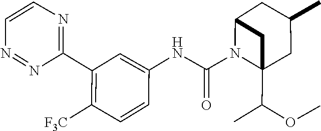 |
| | single unknown enantiomer, second eluting peak |
| 213 | 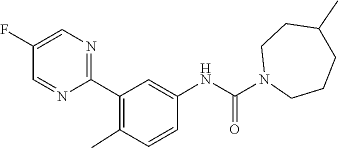 [SMILES: Cc1ccc(NC(=O)N2CCCC(C)CC2)cc1-c1ncc(F)cn1] |
| | mixture of enantiomers |
| 214 | 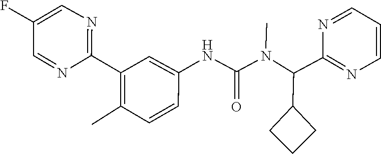 [SMILES: Cc1ccc(NC(=O)N(C)C(c2ncccn2)C2CCC2)cc1-c1ncc(F)cn1] |
| | mixture of enantiomers |
| 215 | 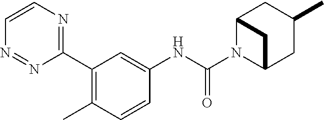 |
| 216 | 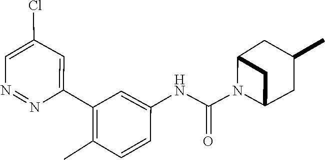 |
| 217 | 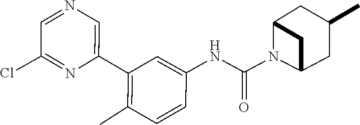 |
| 218 | 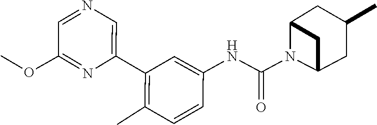 |
| 219 | 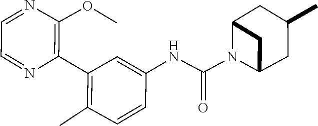 |
| 220 | 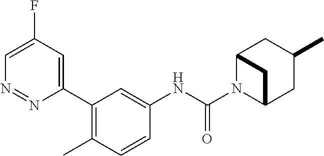 |
| 221 | 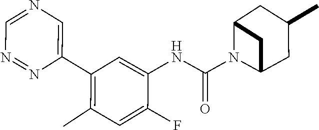 |
| 222 | 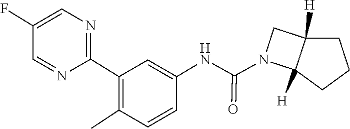 |
| | mixture of enantiomers |
| 223 | 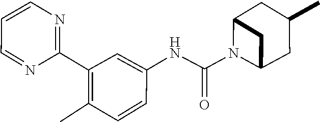 |
| 224 | 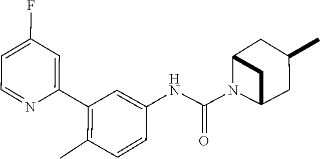 |
| 225 | 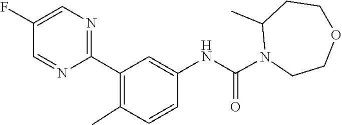 [SMILES: Cc1ccc(NC(=O)N2CCOCCC2C)cc1-c1ncc(F)cn1] |
| | mixture of enantiomers |
| 226 | 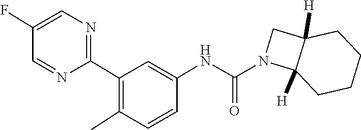 [SMILES: Cc1ccc(NC(=O)N2C[C@@H]3CCCC[C@@H]32)cc1-c1ncc(F)cn1] |
| | mixture of enantiomers |
| 227 | 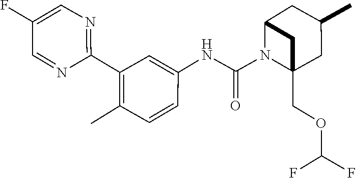 |
| | mixture of enantiomers |
| 228 | 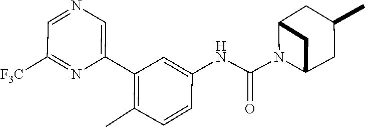 |
| 229 | 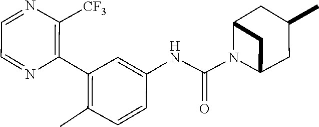 |
| 230 | 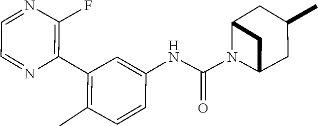 |
| 231 | 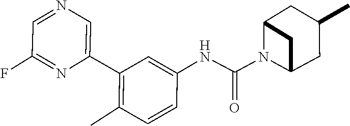 |
| 232 | 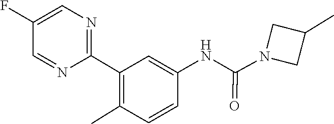 [SMILES: Cc1ccc(NC(=O)N2CC(C)C2)cc1-c1ncc(F)cn1] |
| 233 | 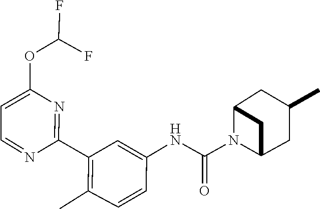 |
| 234 | 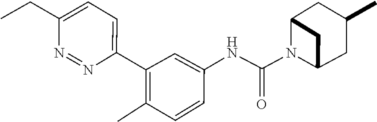 |
| 235 | 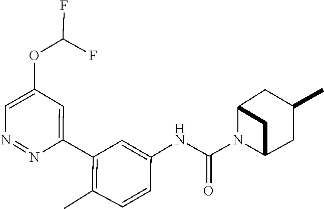 |
| 236 | 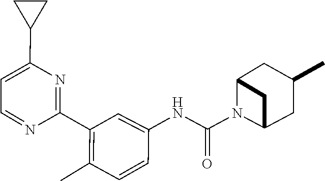 |
| 237 | 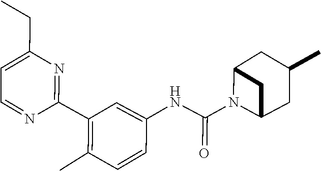 |
| 238 | 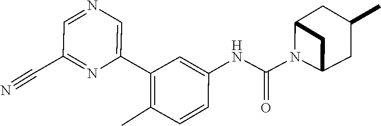 |
| 239 | 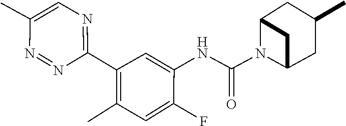 |
| 240 | 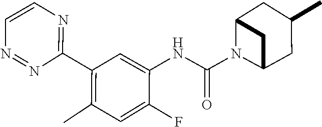 |
| 241 | 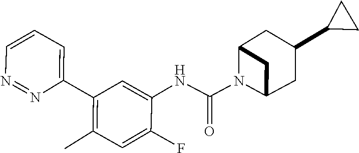 |
| 242 | 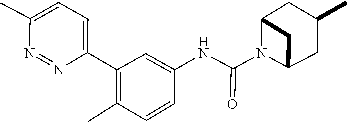 |
| 243 | 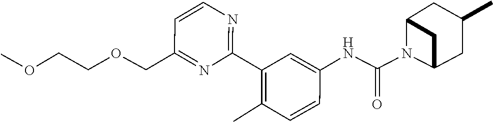 |
| 244 | 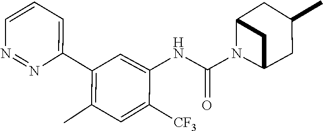 |
| 245 | 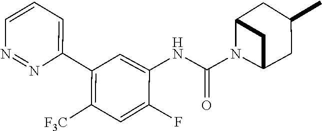 |
| 246 | 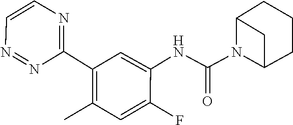 [SMILES: Cc1cc(F)c(NC(=O)N2C3CCCC2C3)cc1-c1nccnn1] |
| 247 | 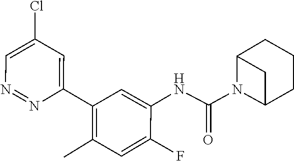 [SMILES: Cc1cc(F)c(NC(=O)N2C3CCCC2C3)cc1-c1cc(Cl)cnn1] |
| 248 | 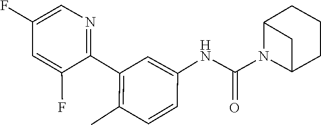 [SMILES: Cc1ccc(NC(=O)N2C3CCCC2C3)cc1-c1ncc(F)cc1F] |
| 249 | 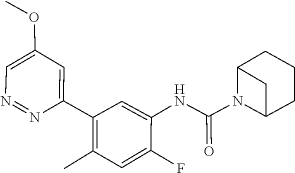 [SMILES: COc1cnnc(-c2cc(NC(=O)N3C4CCCC3C4)c(F)cc2C)c1] |
| 250 | 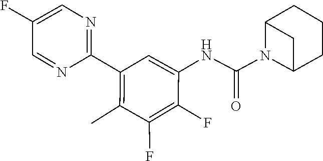 [SMILES: Cc1c(-c2ncc(F)cn2)cc(NC(=O)N2C3CCCC2C3)c(F)c1F] |
| 251 | 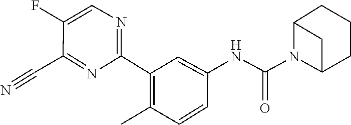 [SMILES: Cc1ccc(NC(=O)N2C3CCCC2C3)cc1-c1ncc(F)c(C#N)n1] |
| 252 | 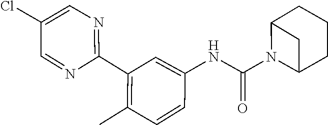 [SMILES: Cc1ccc(NC(=O)N2C3CCCC2C3)cc1-c1ncc(Cl)cn1] |
| 253 | 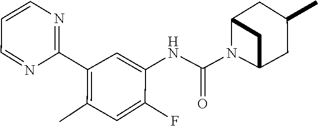 |
| 254 | 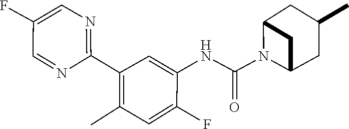 |
| 255 | 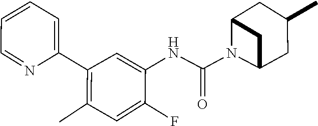 |
| 256 | 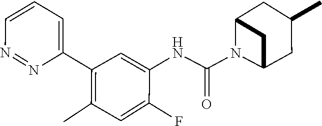 |
| 257 | 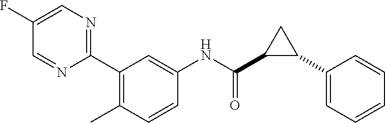 [SMILES: Cc1ccc(NC(=O)[C@H]2C[C@@H]2c2ccccc2)cc1-c1ncc(F)cn1] |
| | mixture of enantiomers |
| 258 | 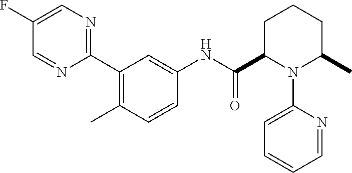 |
| | mixture of enantiomers |
| 259 | 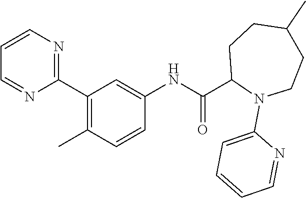 [SMILES: Cc1ccc(NC(=O)C2CCC(C)CCN2c2ccccn2)cc1-c1ncccn1] |
| | mixture of diastereomers |
| 260 | 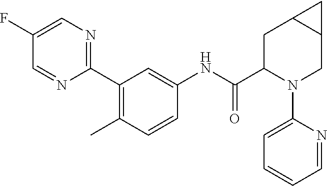 [SMILES: Cc1ccc(NC(=O)C2CC3CC3CN2c2ccccn2)cc1-c1ncc(F)cn1] |
| | mixture of diastereomers |
| 261 | 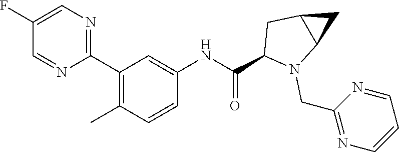 [SMILES: Cc1ccc(NC(=O)[C@H]2C[C@H]3C[C@H]3N2Cc2ncccn2)cc1-c1ncc(F)cn1] |
| 262 | 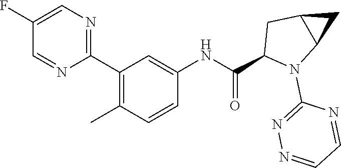 [SMILES: Cc1ccc(NC(=O)[C@H]2C[C@H]3C[C@H]3N2c2nccnn2)cc1-c1ncc(F)cn1] |
| 263 | 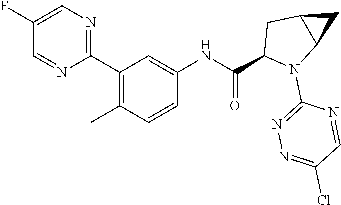 [SMILES: Cc1ccc(NC(=O)[C@H]2C[C@H]3C[C@H]3N2c2ncc(Cl)nn2)cc1-c1ncc(F)cn1] |
| 264 | 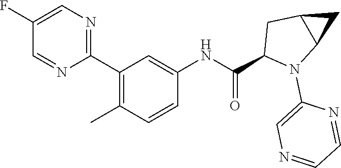 [SMILES: Cc1ccc(NC(=O)[C@H]2C[C@H]3C[C@H]3N2c2cnccn2)cc1-c1ncc(F)cn1] |
| 265 | 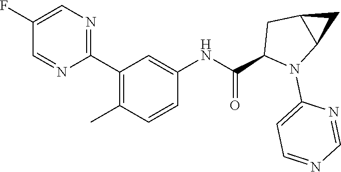 [SMILES: Cc1ccc(NC(=O)[C@H]2C[C@H]3C[C@H]3N2c2ccncn2)cc1-c1ncc(F)cn1] |
| 266 | 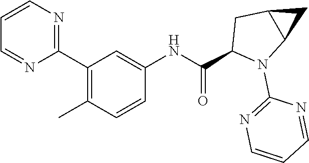 [SMILES: Cc1ccc(NC(=O)[C@H]2C[C@H]3C[C@H]3N2c2ncccn2)cc1-c1ncccn1] |
| 267 | 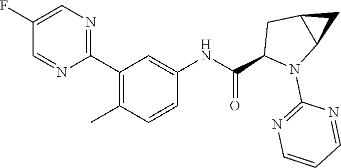 [SMILES: Cc1ccc(NC(=O)[C@H]2C[C@H]3C[C@H]3N2c2ncccn2)cc1-c1ncc(F)cn1] |
| 268 | 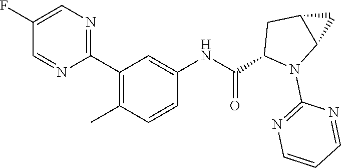 [SMILES: Cc1ccc(NC(=O)[C@@H]2C[C@@H]3C[C@@H]3N2c2ncccn2)cc1-c1ncc(F)cn1] |
| 269 | 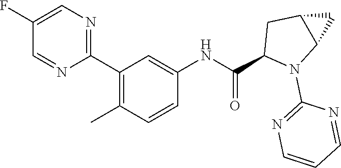 [SMILES: Cc1ccc(NC(=O)[C@H]2C[C@@H]3C[C@@H]3N2c2ncccn2)cc1-c1ncc(F)cn1] |
| 270 | 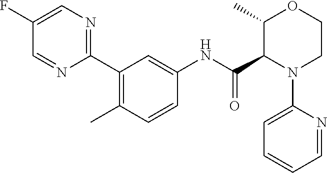 [SMILES: Cc1ccc(NC(=O)[C@H]2[C@H](C)OCCN2c2ccccn2)cc1-c1ncc(F)cn1] |
| 271 | 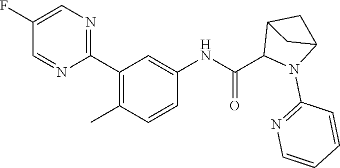 [SMILES: Cc1ccc(NC(=O)C2C3CC(C3)N2c2ccccn2)cc1-c1ncc(F)cn1] |
| | mixture of enantiomers |
| 272 | 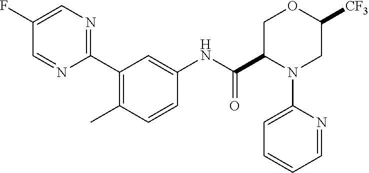 |
| | mixture of enantiomers |
| 273 | 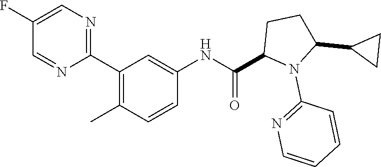 [SMILES: Cc1ccc(NC(=O)[C@H]2CC[C@@H](C3CC3)N2c2ccccn2)cc1-c1ncc(F)cn1] |
| | mixture of enantiomers |
| 274 | 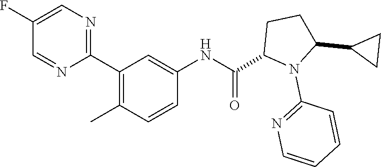 [SMILES: Cc1ccc(NC(=O)[C@@H]2CC[C@@H](C3CC3)N2c2ccccn2)cc1-c1ncc(F)cn1] |
| | mixture of enantiomers |
| 275 | 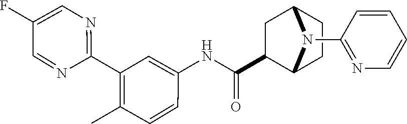 |
| | mixture of enantiomers |
| 276 | 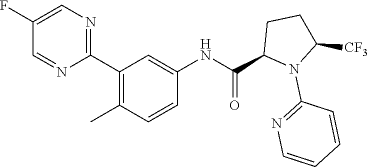 [SMILES: Cc1ccc(NC(=O)[C@H]2CC[C@@H](C(F)(F)F)N2c2ccccn2)cc1-c1ncc(F)cn1] |
| 277 | 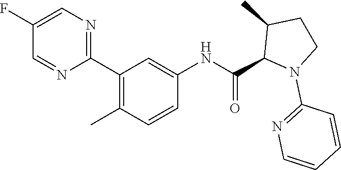 [SMILES: Cc1ccc(NC(=O)[C@H]2[C@@H](C)CCN2c2ccccn2)cc1-c1ncc(F)cn1] |
| 278 | 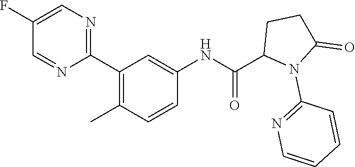 [SMILES: Cc1ccc(NC(=O)C2CCC(=O)N2c2ccccn2)cc1-c1ncc(F)cn1] |
| | mixture of enantiomers |
| 279 | 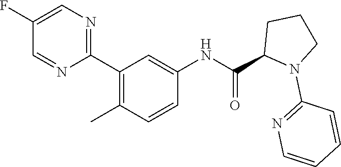 [SMILES: Cc1ccc(NC(=O)[C@H]2CCCN2c2ccccn2)cc1-c1ncc(F)cn1] |
| 280 | 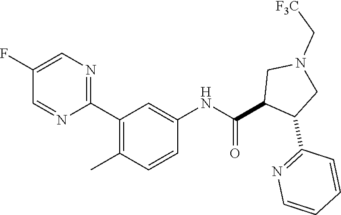 |
| | mixture of enantiomers |
| 281 | 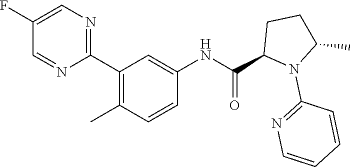 [SMILES: Cc1ccc(NC(=O)[C@H]2CC[C@H](C)N2c2ccccn2)cc1-c1ncc(F)cn1] |
| 282 | 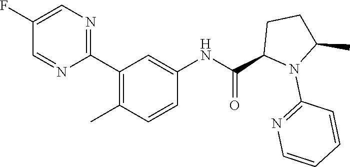 [SMILES: Cc1ccc(NC(=O)[C@H]2CC[C@@H](C)N2c2ccccn2)cc1-c1ncc(F)cn1] |
| 283 | 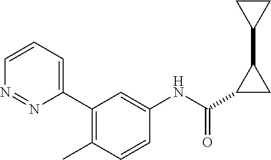 [SMILES: Cc1ccc(NC(=O)[C@H]2C[C@@H]2C2CC2)cc1-c1cccnn1] |
| | mixture of enantiomers |
| 284 | 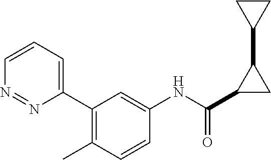 [SMILES: Cc1ccc(NC(=O)[C@@H]2C[C@@H]2C2CC2)cc1-c1cccnn1] |
| | mixture of enantiomers |
| 285 | 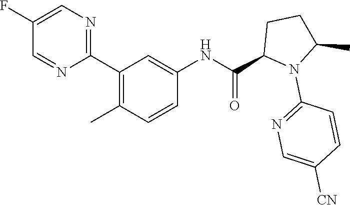 [SMILES: Cc1ccc(NC(=O)[C@H]2CC[C@@H](C)N2c2ccc(C#N)cn2)cc1-c1ncc(F)cn1] |
| 286 | 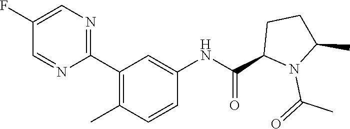 [SMILES: CC(=O)N1[C@H](C)CC[C@@H]1C(=O)Nc1ccc(C)c(-c2ncc(F)cn2)c1] |
| 287 | 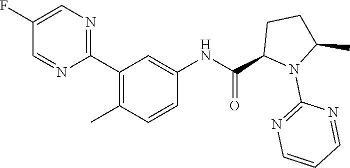 [SMILES: Cc1ccc(NC(=O)[C@H]2CC[C@@H](C)N2c2ncccn2)cc1-c1ncc(F)cn1] |
| 288 | 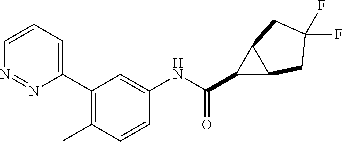 |
| 289 | 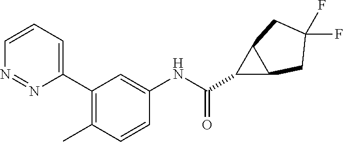 |
| 290 | 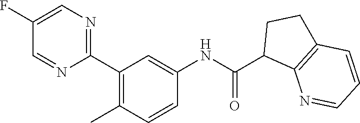 [SMILES: Cc1ccc(NC(=O)C2CCc3cccnc32)cc1-c1ncc(F)cn1] |
| | mixture of enantiomers |
| 291 | 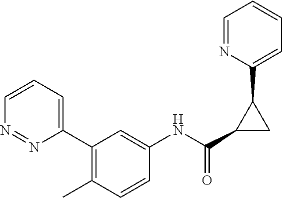 [SMILES: Cc1ccc(NC(=O)[C@@H]2C[C@@H]2c2ccccn2)cc1-c1cccnn1] |
| 292 | 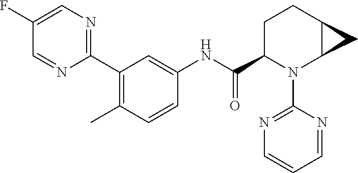 [SMILES: Cc1ccc(NC(=O)[C@H]2CC[C@H]3C[C@H]3N2c2ncccn2)cc1-c1ncc(F)cn1] |
| 293 | 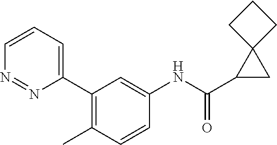 [SMILES: Cc1ccc(NC(=O)C2CC23CCC3)cc1-c1cccnn1] |
| | mixture of enantiomers |
| 294 | 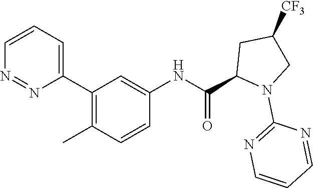 [SMILES: Cc1ccc(NC(=O)[C@H]2C[C@@H](C(F)(F)F)CN2c2ncccn2)cc1-c1cccnn1] |
| 295 | 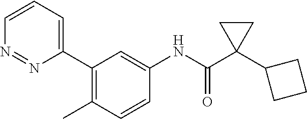 [SMILES: Cc1ccc(NC(=O)C2(C3CCC3)CC2)cc1-c1cccnn1] |
| 296 | 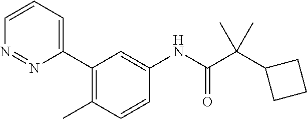 [SMILES: Cc1ccc(NC(=O)C(C)(C)C2CCC2)cc1-c1cccnn1] |
| 297 | 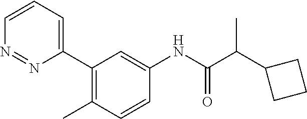 [SMILES: Cc1ccc(NC(=O)C(C)C2CCC2)cc1-c1cccnn1] |
| | mixture of enantiomers |
| 298 | 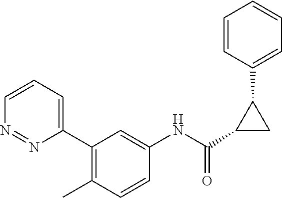 [SMILES: Cc1ccc(NC(=O)[C@H]2C[C@H]2c2ccccc2)cc1-c1cccnn1] |
| 299 | 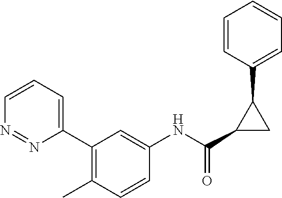 [SMILES: Cc1ccc(NC(=O)[C@@H]2C[C@@H]2c2ccccc2)cc1-c1cccnn1] |
| 300 | 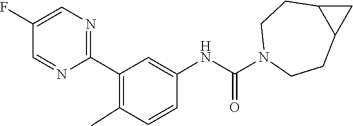 [SMILES: Cc1ccc(NC(=O)N2CCC3CC3CC2)cc1-c1ncc(F)cn1] |
| 301 | 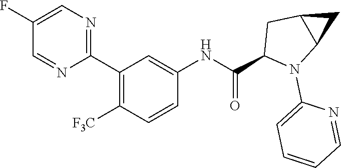 [SMILES: O=C(Nc1ccc(C(F)(F)F)c(-c2ncc(F)cn2)c1)[C@H]1C[C@H]2C[C@H]2N1c1ccccn1] |
| 302 | 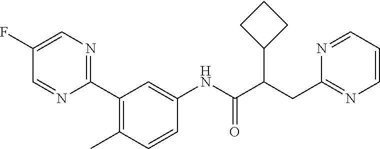 [SMILES: Cc1ccc(NC(=O)C(Cc2ncccn2)C2CCC2)cc1-c1ncc(F)cn1] |
| | mixture of enantiomers |
| 303 | 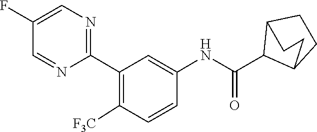 [SMILES: O=C(Nc1ccc(C(F)(F)F)c(-c2ncc(F)cn2)c1)C1C2CCC1CC2] |
| 304 | 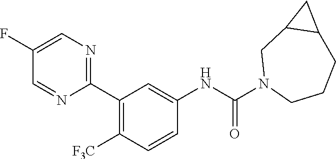 [SMILES: O=C(Nc1ccc(C(F)(F)F)c(-c2ncc(F)cn2)c1)N1CCCC2CC2C1] |
| | mixture of enantiomers |
| 305 | 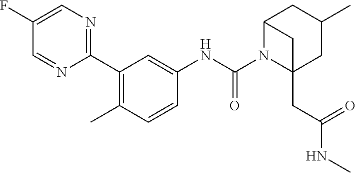 [SMILES: CNC(=O)CC12CC(C)CC(C1)N2C(=O)Nc1ccc(C)c(-c2ncc(F)cn2)c1] |
| | mixture of diastereomers |
| 306 | 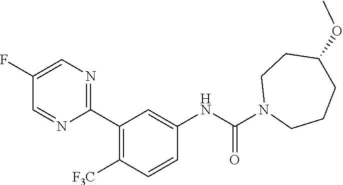 [SMILES: CO[C@@H]1CCCN(C(=O)Nc2ccc(C(F)(F)F)c(-c3ncc(F)cn3)c2)CC1] |
| 307 | 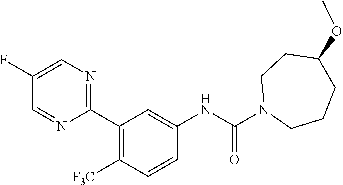 [SMILES: CO[C@H]1CCCN(C(=O)Nc2ccc(C(F)(F)F)c(-c3ncc(F)cn3)c2)CC1] |
| 308 | 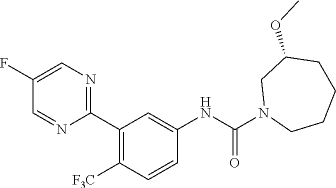 [SMILES: CO[C@@H]1CCCCN(C(=O)Nc2ccc(C(F)(F)F)c(-c3ncc(F)cn3)c2)C1] |
| 309 | 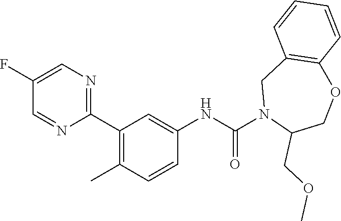 [SMILES: COCC1COc2ccccc2CN1C(=O)Nc1ccc(C)c(-c2ncc(F)cn2)c1] |
| | mixture of enantiomers |
| 310 | 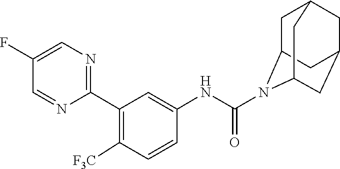 [SMILES: O=C(Nc1ccc(C(F)(F)F)c(-c2ncc(F)cn2)c1)N1C2CC3CC(C2)CC1C3] |
| 311 | 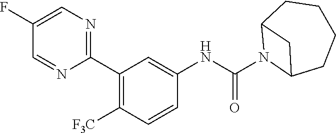 [SMILES: O=C(Nc1ccc(C(F)(F)F)c(-c2ncc(F)cn2)c1)N1C2CCCCC1C2] |
| 312 | 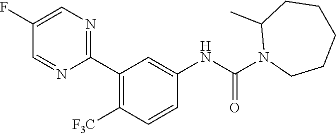 [SMILES: CC1CCCCCN1C(=O)Nc1ccc(C(F)(F)F)c(-c2ncc(F)cn2)c1] |
| | mixture of enantiomers |
| 313 | 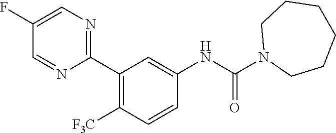 [SMILES: O=C(Nc1ccc(C(F)(F)F)c(-c2ncc(F)cn2)c1)N1CCCCCC1] |
| 314 | 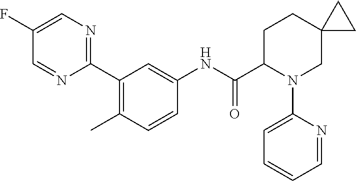 [SMILES: Cc1ccc(NC(=O)C2CCC3(CC3)CN2c2ccccn2)cc1-c1ncc(F)cn1] |
| | mixture of enantiomers |
| 315 | 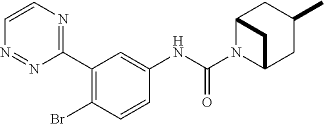 |
| 316 | 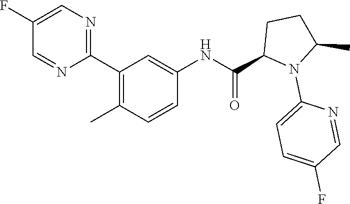 [SMILES: Cc1ccc(NC(=O)[C@H]2CC[C@@H](C)N2c2ccc(F)cn2)cc1-c1ncc(F)cn1] |
| 317 | 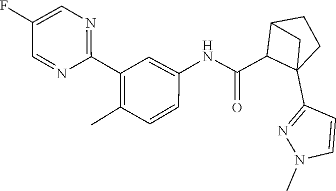 [SMILES: Cc1ccc(NC(=O)C2C3CCC2(c2ccn(C)n2)C3)cc1-c1ncc(F)cn1] |
| | mixture of diastereomers |
| 318 | 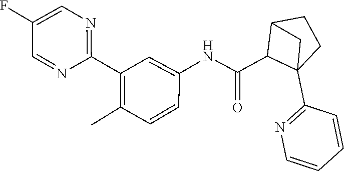 [SMILES: Cc1ccc(NC(=O)C2C3CCC2(c2ccccn2)C3)cc1-c1ncc(F)cn1] |
| | mixture of diastereomers |
| 319 | 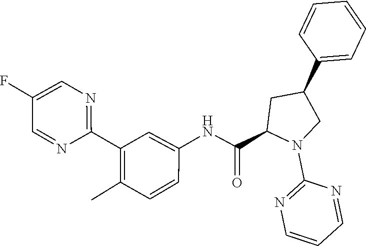 [SMILES: Cc1ccc(NC(=O)[C@H]2C[C@@H](c3ccccc3)CN2c2ncccn2)cc1-c1ncc(F)cn1] |
| 320 | 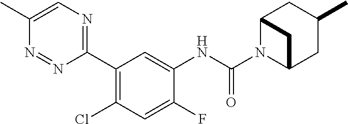 |
| 321 | 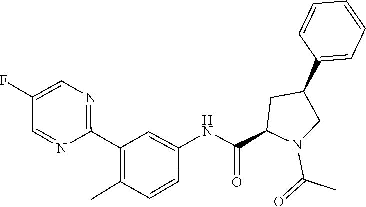 [SMILES: CC(=O)N1C[C@H](c2ccccc2)C[C@@H]1C(=O)Nc1ccc(C)c(-c2ncc(F)cn2)c1] |
| 322 | 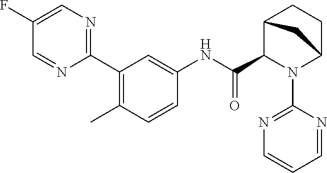 [SMILES: Cc1ccc(NC(=O)[C@H]2[C@@H]3CC[C@@H](C3)N2c2ncccn2)cc1-c1ncc(F)cn1] |
| 323 | 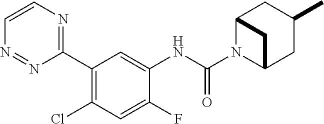 |
| 324 | 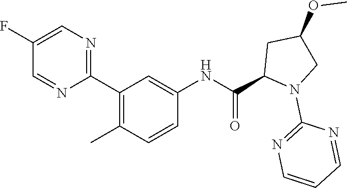 [SMILES: CO[C@@H]1C[C@H](C(=O)Nc2ccc(C)c(-c3ncc(F)cn3)c2)N(c2ncccn2)C1] |
| 325 | 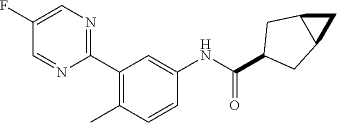 |
| 326 | 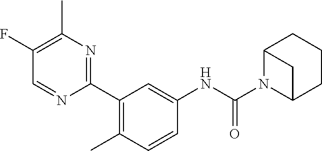 [SMILES: Cc1ccc(NC(=O)N2C3CCCC2C3)cc1-c1ncc(F)c(C)n1] |
| 327 | 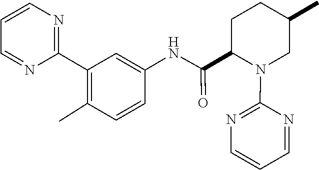 [SMILES: Cc1ccc(NC(=O)[C@H]2CC[C@@H](C)CN2c2ncccn2)cc1-c1ncccn1] |
| | mixture of enantiomers |
| 328 | 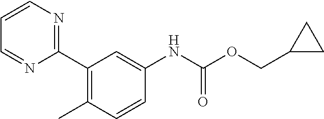 [SMILES: Cc1ccc(NC(=O)OCC2CC2)cc1-c1ncccn1] |

In certain embodiments, provided is a compound selected from Table 2 or a pharmaceutically acceptable salt thereof.

TABLE 2
Structure

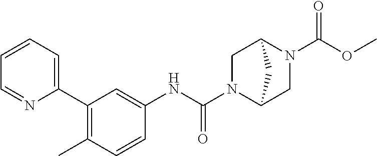 [SMILES: COC(=O)N1C[C@@H]2C[C@H]1CN2C(=O)Nc1ccc(C)c(-c2ccccn2)c1]

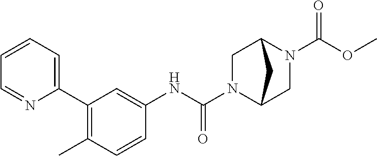 [SMILES: COC(=O)N1C[C@H]2C[C@@H]1CN2C(=O)Nc1ccc(C)c(-c2ccccn2)c1]

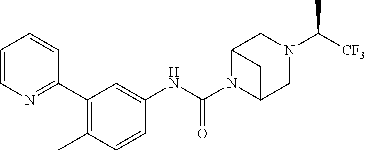 [SMILES: Cc1ccc(NC(=O)N2C3CC2CN([C@@H](C)C(F)(F)F)C3)cc1-c1ccccn1]

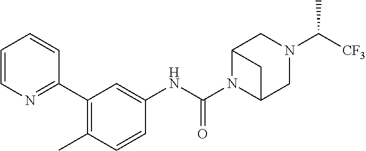 [SMILES: Cc1ccc(NC(=O)N2C3CC2CN([C@H](C)C(F)(F)F)C3)cc1-c1ccccn1]

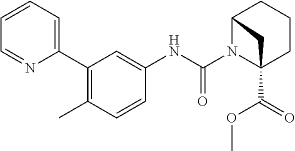

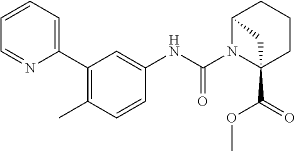

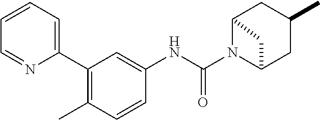 [SMILES: Cc1ccc(NC(=O)N2[C@@H]3C[C@H](C)C[C@H]2C3)cc1-c1ccccn1]

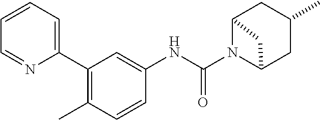 [SMILES: Cc1ccc(NC(=O)N2[C@@H]3C[C@@H](C)C[C@H]2C3)cc1-c1ccccn1]

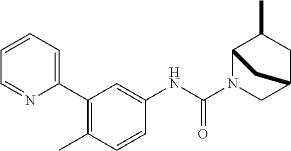

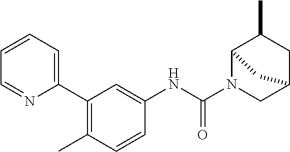

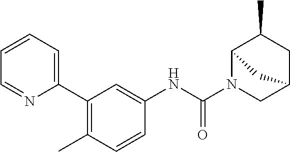 [SMILES: Cc1ccc(NC(=O)N2C[C@H]3C[C@@H]2[C@@H](C)C3)cc1-c1ccccn1]

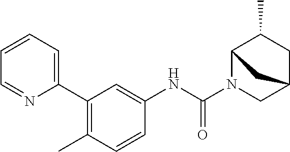 [SMILES: Cc1ccc(NC(=O)N2C[C@H]3C[C@@H](C)[C@@H]2C3)cc1-c1ccccn1]

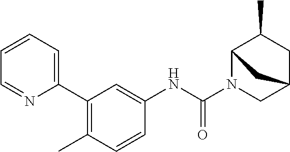 [SMILES: Cc1ccc(NC(=O)N2C[C@H]3C[C@H](C)[C@@H]2C3)cc1-c1ccccn1]

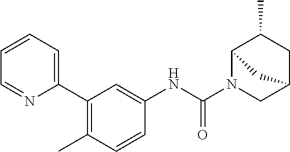 [SMILES: Cc1ccc(NC(=O)N2C[C@@H]3C[C@@H](C)[C@H]2C3)cc1-c1ccccn1]

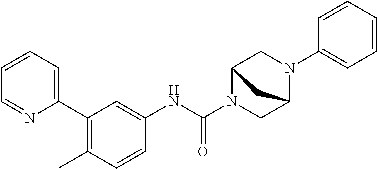 [SMILES: Cc1ccc(NC(=O)N2C[C@@H]3C[C@H]2CN3c2ccccc2)cc1-c1ccccn1]

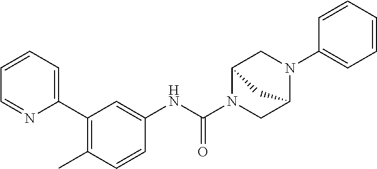 [SMILES: Cc1ccc(NC(=O)N2C[C@H]3C[C@@H]2CN3c2ccccc2)cc1-c1ccccn1]

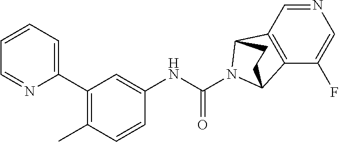 [SMILES: Cc1ccc(NC(=O)N2[C@@H]3CC[C@H]2c2cncc(F)c23)cc1-c1ccccn1]

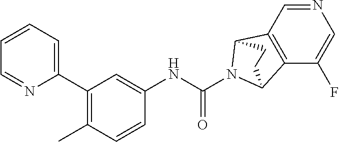 [SMILES: Cc1ccc(NC(=O)N2[C@@H]3CC[C@H]2c2c(F)cncc23)cc1-c1ccccn1]

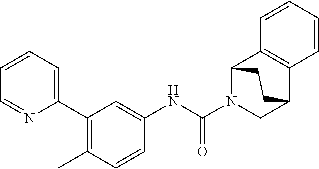 [SMILES: Cc1ccc(NC(=O)N2C[C@@H]3CC[C@H]2c2ccccc23)cc1-c1ccccn1]

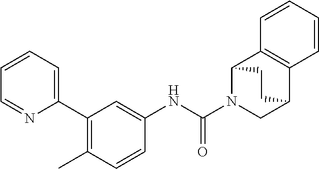 [SMILES: Cc1ccc(NC(=O)N2C[C@H]3CC[C@@H]2c2ccccc23)cc1-c1ccccn1]

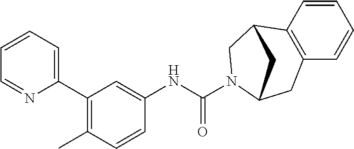 [SMILES: Cc1ccc(NC(=O)N2C[C@H]3C[C@@H]2Cc2ccccc23)cc1-c1ccccn1]

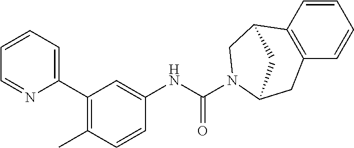 [SMILES: Cc1ccc(NC(=O)N2C[C@@H]3C[C@H]2Cc2ccccc23)cc1-c1ccccn1]

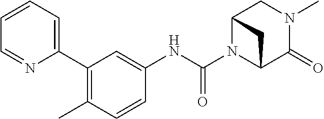 [SMILES: Cc1ccc(NC(=O)N2[C@H]3C[C@@H]2C(=O)N(C)C3)cc1-c1ccccn1]

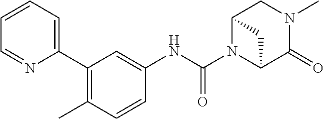 [SMILES: Cc1ccc(NC(=O)N2[C@@H]3C[C@H]2C(=O)N(C)C3)cc1-c1ccccn1]

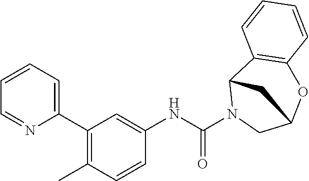 [SMILES: Cc1ccc(NC(=O)N2C[C@@H]3C[C@H]2c2ccccc2O3)cc1-c1ccccn1]

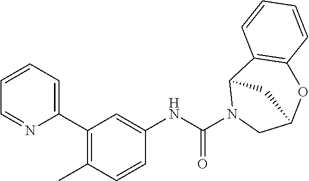 [SMILES: Cc1ccc(NC(=O)N2C[C@H]3C[C@@H]2c2ccccc2O3)cc1-c1ccccn1]

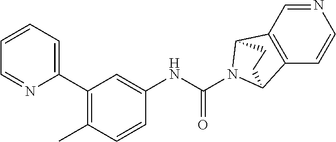 [SMILES: Cc1ccc(NC(=O)N2[C@@H]3CC[C@H]2c2ccncc23)cc1-c1ccccn1]

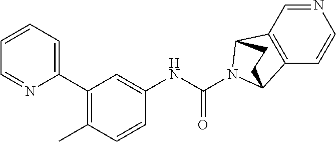 [SMILES: Cc1ccc(NC(=O)N2[C@@H]3CC[C@H]2c2cnccc23)cc1-c1ccccn1]

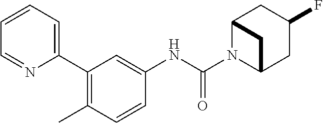

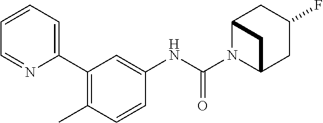

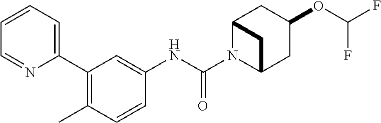

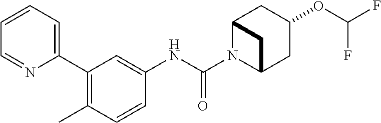

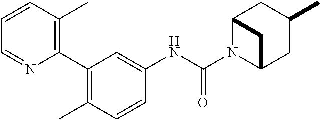

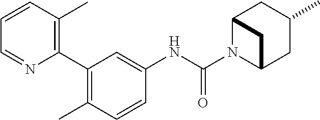

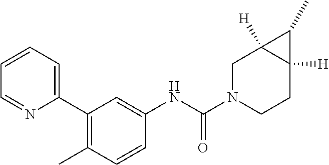 [SMILES: Cc1ccc(NC(=O)N2CC[C@@H]3[C@@H](C)[C@@H]3C2)cc1-c1ccccn1]

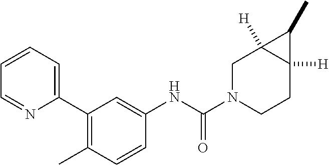 [SMILES: Cc1ccc(NC(=O)N2CC[C@@H]3[C@H](C)[C@@H]3C2)cc1-c1ccccn1]

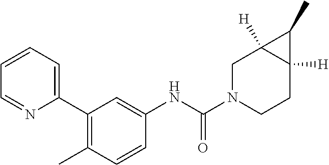 [SMILES: Cc1ccc(NC(=O)N2CC[C@@H]3[C@H](C)[C@@H]3C2)cc1-c1ccccn1]

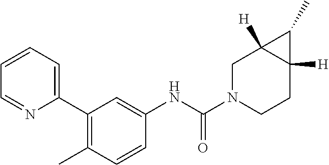 [SMILES: Cc1ccc(NC(=O)N2CC[C@H]3[C@@H](C)[C@H]3C2)cc1-c1ccccn1]

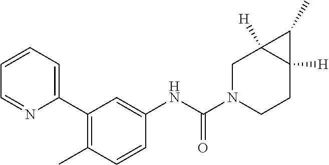 [SMILES: Cc1ccc(NC(=O)N2CC[C@@H]3[C@@H](C)[C@@H]3C2)cc1-c1ccccn1]

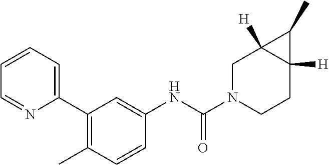 [SMILES: Cc1ccc(NC(=O)N2CC[C@H]3[C@H](C)[C@H]3C2)cc1-c1ccccn1]

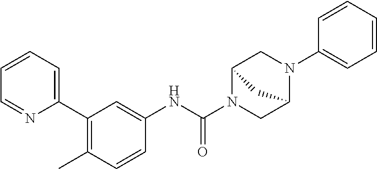 [SMILES: Cc1ccc(NC(=O)N2C[C@H]3C[C@@H]2CN3c2ccccc2)cc1-c1ccccn1]

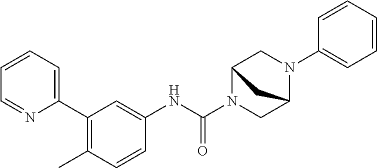 [SMILES: Cc1ccc(NC(=O)N2C[C@@H]3C[C@H]2CN3c2ccccc2)cc1-c1ccccn1]

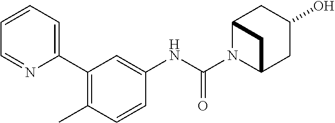

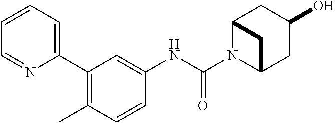

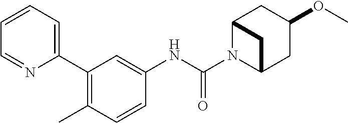

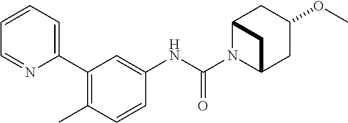

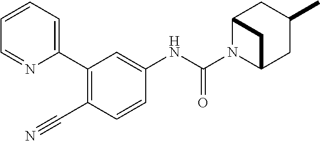

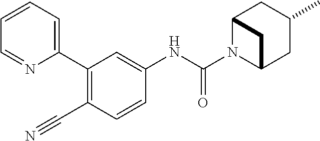

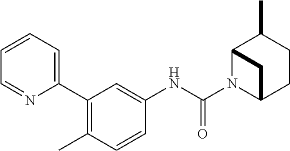

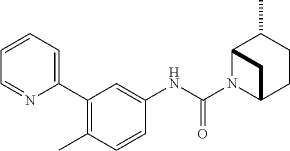

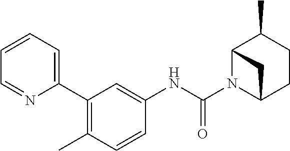 [SMILES: Cc1ccc(NC(=O)N2[C@H]3CC[C@H](C)[C@@H]2C3)cc1-c1ccccn1]

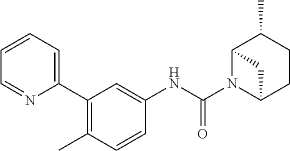 [SMILES: Cc1ccc(NC(=O)N2[C@@H]3CC[C@@H](C)[C@H]2C3)cc1-c1ccccn1]

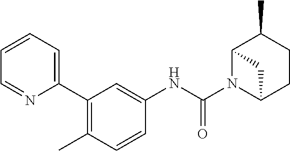 [SMILES: Cc1ccc(NC(=O)N2[C@@H]3CC[C@H](C)[C@H]2C3)cc1-c1ccccn1]

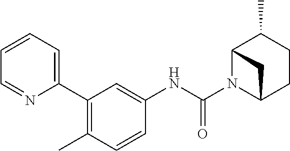 [SMILES: Cc1ccc(NC(=O)N2[C@H]3CC[C@@H](C)[C@@H]2C3)cc1-c1ccccn1]

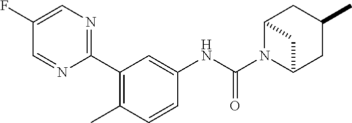

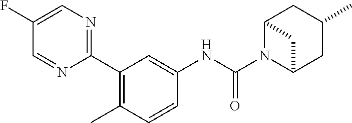

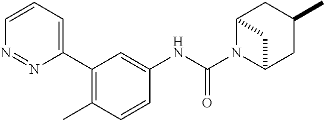

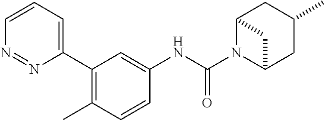

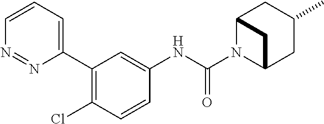

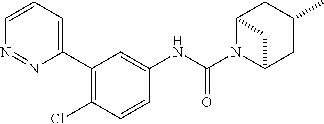

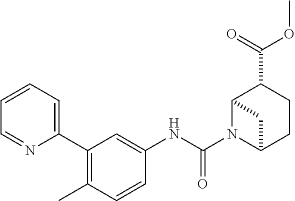

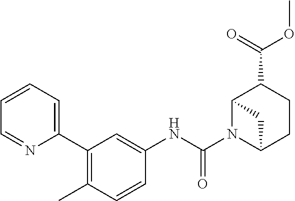

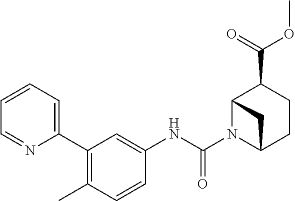 [SMILES: COC(=O)[C@H]1CC[C@H]2C[C@@H]1N2C(=O)Nc1ccc(C)c(-c2ccccn2)c1]

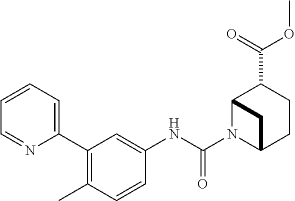

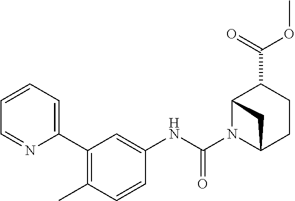 [SMILES: COC(=O)[C@@H]1CC[C@H]2C[C@@H]1N2C(=O)Nc1ccc(C)c(-c2ccccn2)c1]

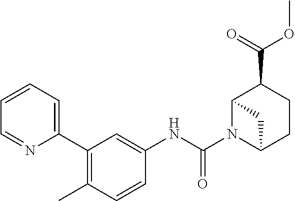 [SMILES: COC(=O)[C@H]1CC[C@@H]2C[C@H]1N2C(=O)Nc1ccc(C)c(-c2ccccn2)c1]

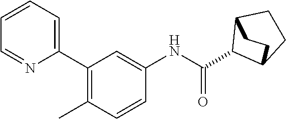

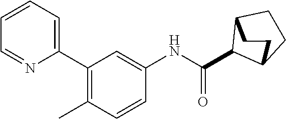

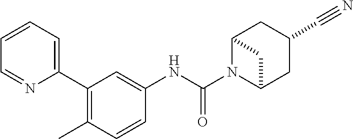

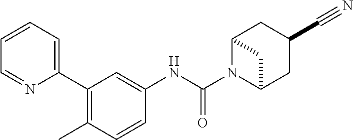

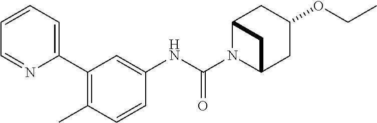

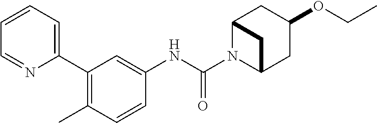

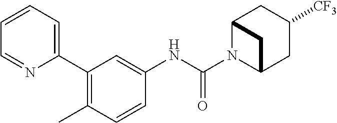

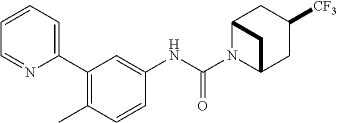

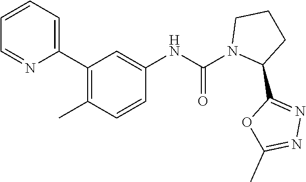 [SMILES: Cc1nnc([C@@H]2CCCN2C(=O)Nc2ccc(C)c(-c3ccccn3)c2)o1]

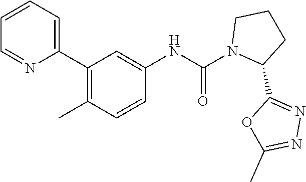 [SMILES: Cc1nnc([C@H]2CCCN2C(=O)Nc2ccc(C)c(-c3ccccn3)c2)o1]

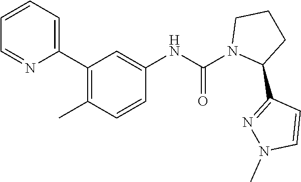 [SMILES: Cc1ccc(NC(=O)N2CCC[C@H]2c2ccn(C)n2)cc1-c1ccccn1]

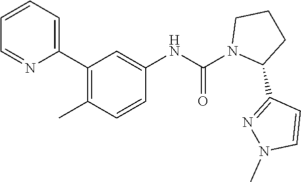 [SMILES: Cc1ccc(NC(=O)N2CCC[C@@H]2c2ccn(C)n2)cc1-c1ccccn1]

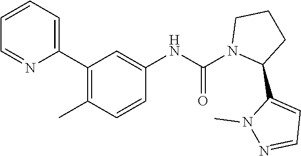 [SMILES: Cc1ccc(NC(=O)N2CCC[C@H]2c2ccnn2C)cc1-c1ccccn1]

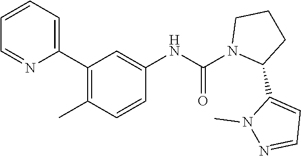 [SMILES: Cc1ccc(NC(=O)N2CCC[C@@H]2c2ccnn2C)cc1-c1ccccn1]

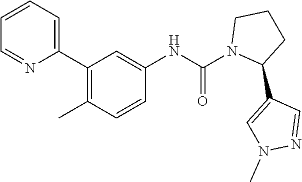 [SMILES: Cc1ccc(NC(=O)N2CCC[C@H]2c2cnn(C)c2)cc1-c1ccccn1]

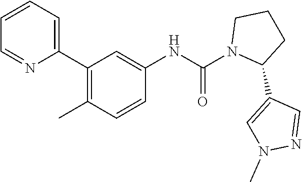 [SMILES: Cc1ccc(NC(=O)N2CCC[C@@H]2c2cnn(C)c2)cc1-c1ccccn1]

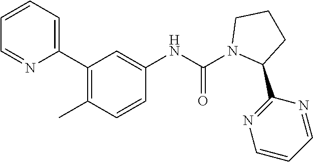 [SMILES: Cc1ccc(NC(=O)N2CCC[C@H]2c2ncccn2)cc1-c1ccccn1]

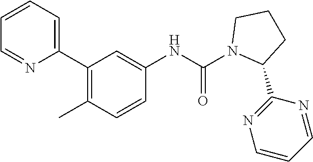 [SMILES: Cc1ccc(NC(=O)N2CCC[C@@H]2c2ncccn2)cc1-c1ccccn1]

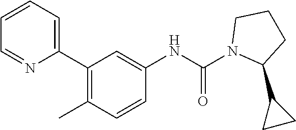 [SMILES: Cc1ccc(NC(=O)N2CCC[C@H]2C2CC2)cc1-c1ccccn1]

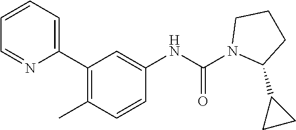 [SMILES: Cc1ccc(NC(=O)N2CCC[C@@H]2C2CC2)cc1-c1ccccn1]

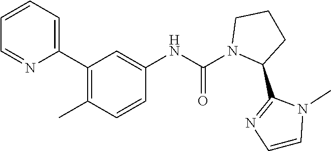 [SMILES: Cc1ccc(NC(=O)N2CCC[C@H]2c2nccn2C)cc1-c1ccccn1]

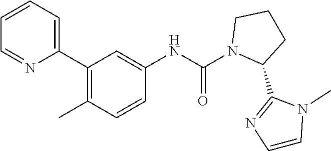 [SMILES: Cc1ccc(NC(=O)N2CCC[C@@H]2c2nccn2C)cc1-c1ccccn1]

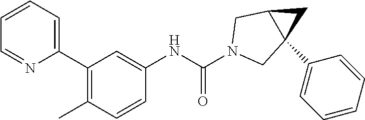 [SMILES: Cc1ccc(NC(=O)N2C[C@H]3C[C@@]3(c3ccccc3)C2)cc1-c1ccccn1]

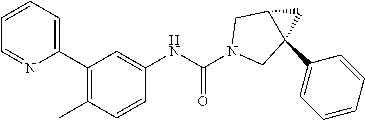 [SMILES: Cc1ccc(NC(=O)N2C[C@@H]3C[C@]3(c3ccccc3)C2)cc1-c1ccccn1]

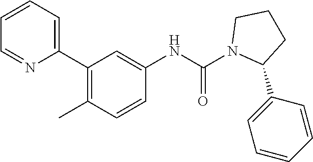 [SMILES: Cc1ccc(NC(=O)N2CCC[C@@H]2c2ccccc2)cc1-c1ccccn1]

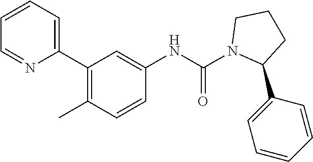 [SMILES: Cc1ccc(NC(=O)N2CCC[C@H]2c2ccccc2)cc1-c1ccccn1]

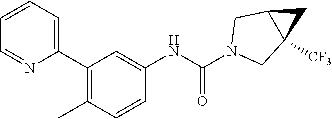 [SMILES: Cc1ccc(NC(=O)N2C[C@H]3C[C@@]3(C(F)(F)F)C2)cc1-c1ccccn1]

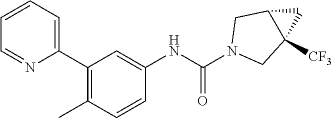 [SMILES: Cc1ccc(NC(=O)N2C[C@@H]3C[C@]3(C(F)(F)F)C2)cc1-c1ccccn1]

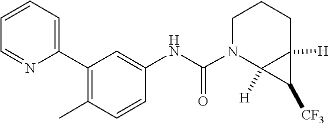 [SMILES: Cc1ccc(NC(=O)N2CCC[C@@H]3[C@H]2[C@@H]3C(F)(F)F)cc1-c1ccccn1]

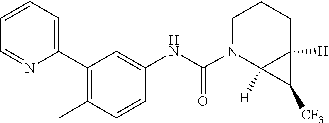 [SMILES: Cc1ccc(NC(=O)N2CCC[C@@H]3[C@H]2[C@@H]3C(F)(F)F)cc1-c1ccccn1]

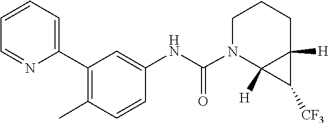 [SMILES: Cc1ccc(NC(=O)N2CCC[C@H]3[C@@H]2[C@H]3C(F)(F)F)cc1-c1ccccn1]

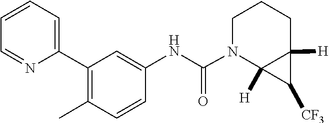 [SMILES: Cc1ccc(NC(=O)N2CCC[C@H]3[C@@H]2[C@@H]3C(F)(F)F)cc1-c1ccccn1]

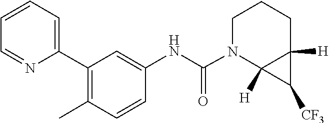 [SMILES: Cc1ccc(NC(=O)N2CCC[C@H]3[C@@H]2[C@@H]3C(F)(F)F)cc1-c1ccccn1]

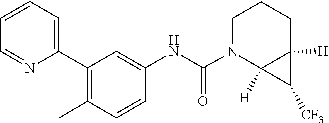 [SMILES: Cc1ccc(NC(=O)N2CCC[C@@H]3[C@H]2[C@H]3C(F)(F)F)cc1-c1ccccn1]

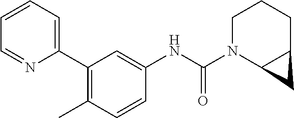 [SMILES: Cc1ccc(NC(=O)N2CCC[C@H]3C[C@H]32)cc1-c1ccccn1]

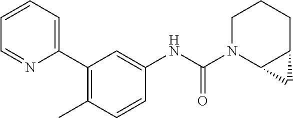 [SMILES: Cc1ccc(NC(=O)N2CCC[C@@H]3C[C@@H]32)cc1-c1ccccn1]

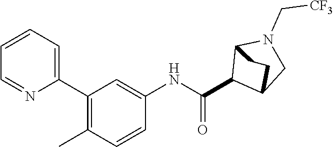 [SMILES: Cc1ccc(NC(=O)[C@@H]2[C@@H]3CC[C@H]2N(CC(F)(F)F)C3)cc1-c1ccccn1]

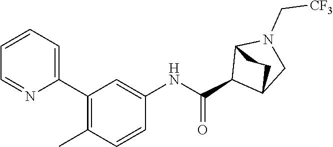 [SMILES: Cc1ccc(NC(=O)[C@@H]2[C@@H]3CC[C@H]2N(CC(F)(F)F)C3)cc1-c1ccccn1]

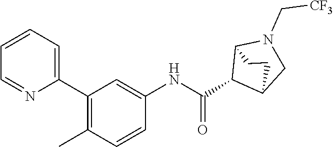 [SMILES: Cc1ccc(NC(=O)[C@H]2[C@H]3CC[C@@H]2N(CC(F)(F)F)C3)cc1-c1ccccn1]

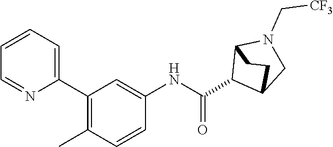 [SMILES: Cc1ccc(NC(=O)[C@H]2[C@@H]3CC[C@H]2N(CC(F)(F)F)C3)cc1-c1ccccn1]

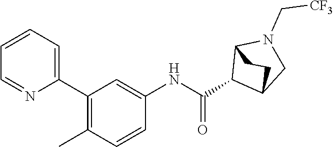 [SMILES: Cc1ccc(NC(=O)[C@H]2[C@@H]3CC[C@H]2N(CC(F)(F)F)C3)cc1-c1ccccn1]

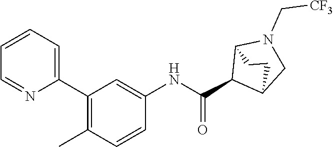 [SMILES: Cc1ccc(NC(=O)[C@@H]2[C@H]3CC[C@@H]2N(CC(F)(F)F)C3)cc1-c1ccccn1]

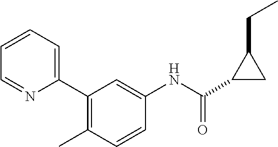 [SMILES: CC[C@H]1C[C@@H]1C(=O)Nc1ccc(C)c(-c2ccccn2)c1]

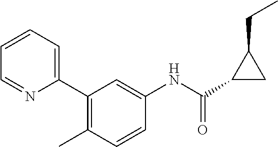 [SMILES: CC[C@H]1C[C@@H]1C(=O)Nc1ccc(C)c(-c2ccccn2)c1]

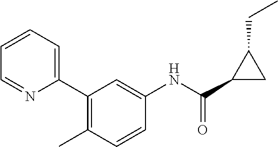 [SMILES: CC[C@@H]1C[C@H]1C(=O)Nc1ccc(C)c(-c2ccccn2)c1]

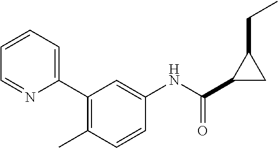 [SMILES: CC[C@H]1C[C@H]1C(=O)Nc1ccc(C)c(-c2ccccn2)c1]

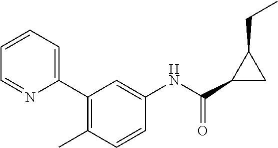 [SMILES: CC[C@H]1C[C@H]1C(=O)Nc1ccc(C)c(-c2ccccn2)c1]

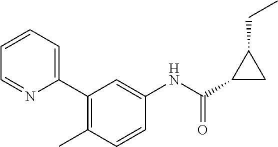 [SMILES: CC[C@@H]1C[C@@H]1C(=O)Nc1ccc(C)c(-c2ccccn2)c1]

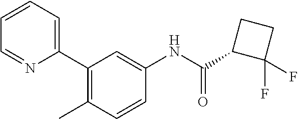 [SMILES: Cc1ccc(NC(=O)[C@@H]2CCC2(F)F)cc1-c1ccccn1]

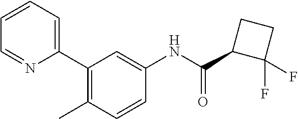 [SMILES: Cc1ccc(NC(=O)[C@H]2CCC2(F)F)cc1-c1ccccn1]

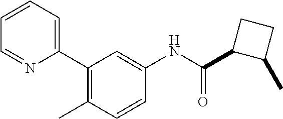

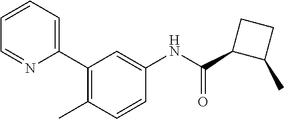 [SMILES: Cc1ccc(NC(=O)[C@H]2CC[C@H]2C)cc1-c1ccccn1]

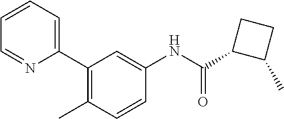 [SMILES: Cc1ccc(NC(=O)[C@@H]2CC[C@@H]2C)cc1-c1ccccn1]

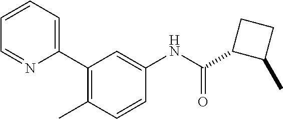

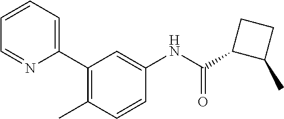 [SMILES: Cc1ccc(NC(=O)[C@@H]2CC[C@H]2C)cc1-c1ccccn1]

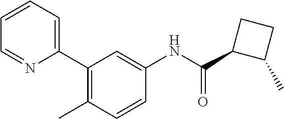 [SMILES: Cc1ccc(NC(=O)[C@H]2CC[C@@H]2C)cc1-c1ccccn1]

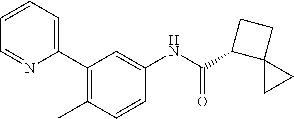 [SMILES: Cc1ccc(NC(=O)[C@@H]2CCC23CC3)cc1-c1ccccn1]

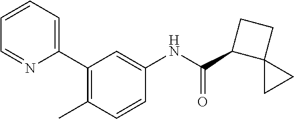 [SMILES: Cc1ccc(NC(=O)[C@H]2CCC23CC3)cc1-c1ccccn1]

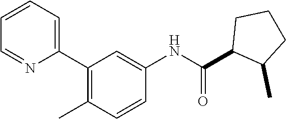

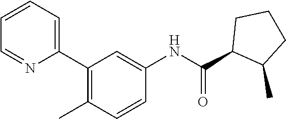 [SMILES: Cc1ccc(NC(=O)[C@H]2CCC[C@H]2C)cc1-c1ccccn1]

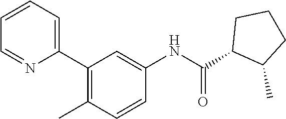 [SMILES: Cc1ccc(NC(=O)[C@@H]2CCC[C@@H]2C)cc1-c1ccccn1]

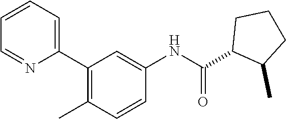

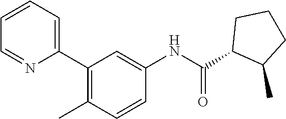 [SMILES: Cc1ccc(NC(=O)[C@@H]2CCC[C@H]2C)cc1-c1ccccn1]

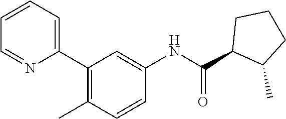 [SMILES: Cc1ccc(NC(=O)[C@H]2CCC[C@@H]2C)cc1-c1ccccn1]

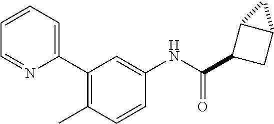

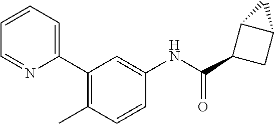 [SMILES: Cc1ccc(NC(=O)[C@@H]2C[C@H]3C[C@H]32)cc1-c1ccccn1]

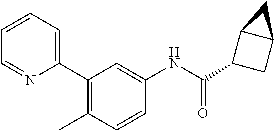 [SMILES: Cc1ccc(NC(=O)[C@H]2C[C@@H]3C[C@@H]32)cc1-c1ccccn1]

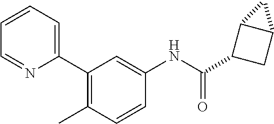

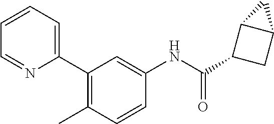 [SMILES: Cc1ccc(NC(=O)[C@H]2C[C@H]3C[C@H]32)cc1-c1ccccn1]

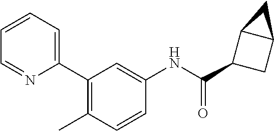 [SMILES: Cc1ccc(NC(=O)[C@@H]2C[C@@H]3C[C@@H]32)cc1-c1ccccn1]

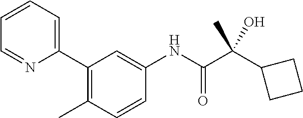 [SMILES: Cc1ccc(NC(=O)[C@](C)(O)C2CCC2)cc1-c1ccccn1]

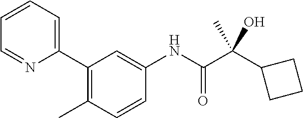 [SMILES: Cc1ccc(NC(=O)[C@@](C)(O)C2CCC2)cc1-c1ccccn1]

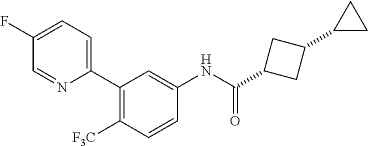 [SMILES: O=C(Nc1ccc(C(F)(F)F)c(-c2ccc(F)cn2)c1)[C@H]1C[C@@H](C2CC2)C1]

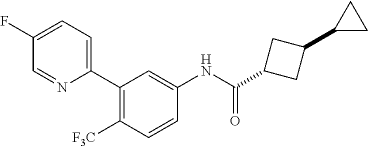 [SMILES: O=C(Nc1ccc(C(F)(F)F)c(-c2ccc(F)cn2)c1)[C@H]1C[C@H](C2CC2)C1]

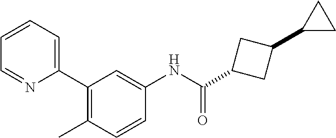 [SMILES: Cc1ccc(NC(=O)[C@H]2C[C@H](C3CC3)C2)cc1-c1ccccn1]

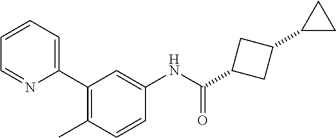 [SMILES: Cc1ccc(NC(=O)[C@H]2C[C@@H](C3CC3)C2)cc1-c1ccccn1]

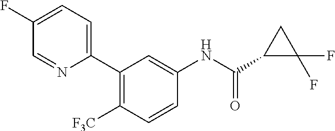 [SMILES: O=C(Nc1ccc(C(F)(F)F)c(-c2ccc(F)cn2)c1)[C@@H]1CC1(F)F]

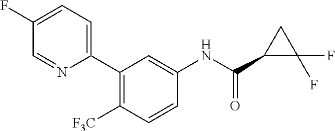 [SMILES: O=C(Nc1ccc(C(F)(F)F)c(-c2ccc(F)cn2)c1)[C@H]1CC1(F)F]

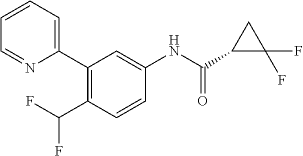 [SMILES: O=C(Nc1ccc(C(F)F)c(-c2ccccn2)c1)[C@@H]1CC1(F)F]

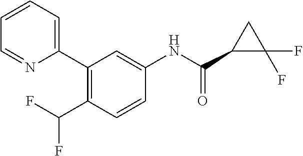 [SMILES: O=C(Nc1ccc(C(F)F)c(-c2ccccn2)c1)[C@H]1CC1(F)F]

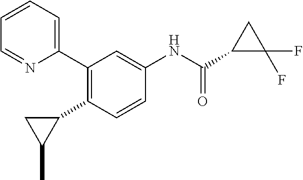 [SMILES: C[C@H]1C[C@@H]1c1ccc(NC(=O)[C@@H]2CC2(F)F)cc1-c1ccccn1]

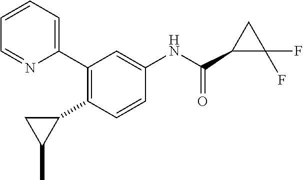 [SMILES: C[C@H]1C[C@@H]1c1ccc(NC(=O)[C@H]2CC2(F)F)cc1-c1ccccn1]

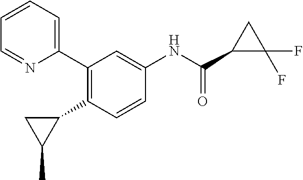 [SMILES: C[C@H]1C[C@@H]1c1ccc(NC(=O)[C@H]2CC2(F)F)cc1-c1ccccn1]

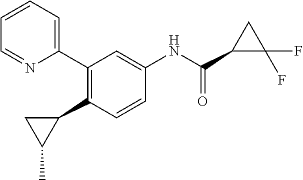 [SMILES: C[C@@H]1C[C@H]1c1ccc(NC(=O)[C@H]2CC2(F)F)cc1-c1ccccn1]

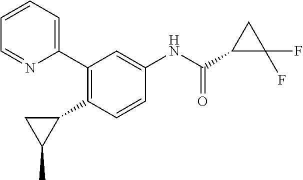 [SMILES: C[C@H]1C[C@@H]1c1ccc(NC(=O)[C@@H]2CC2(F)F)cc1-c1ccccn1]

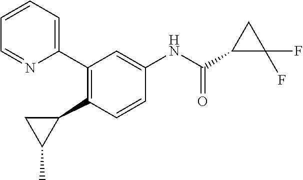 [SMILES: C[C@@H]1C[C@H]1c1ccc(NC(=O)[C@@H]2CC2(F)F)cc1-c1ccccn1]

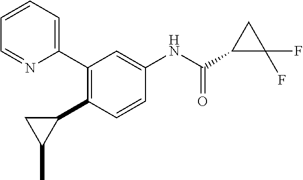 [SMILES: C[C@H]1C[C@H]1c1ccc(NC(=O)[C@@H]2CC2(F)F)cc1-c1ccccn1]

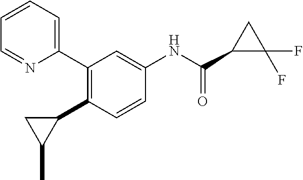 [SMILES: C[C@H]1C[C@H]1c1ccc(NC(=O)[C@H]2CC2(F)F)cc1-c1ccccn1]

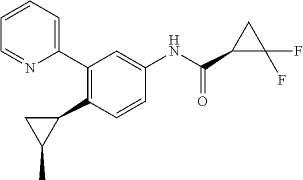 [SMILES: C[C@H]1C[C@H]1c1ccc(NC(=O)[C@H]2CC2(F)F)cc1-c1ccccn1]

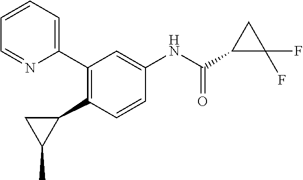 [SMILES: C[C@H]1C[C@H]1c1ccc(NC(=O)[C@@H]2CC2(F)F)cc1-c1ccccn1]

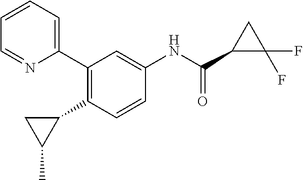 [SMILES: C[C@@H]1C[C@@H]1c1ccc(NC(=O)[C@H]2CC2(F)F)cc1-c1ccccn1]

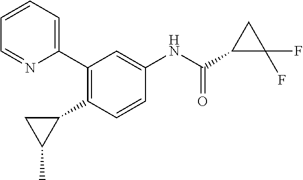 [SMILES: C[C@@H]1C[C@@H]1c1ccc(NC(=O)[C@@H]2CC2(F)F)cc1-c1ccccn1]

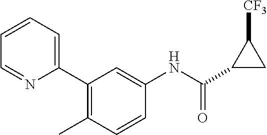 [SMILES: Cc1ccc(NC(=O)[C@H]2C[C@@H]2C(F)(F)F)cc1-c1ccccn1]

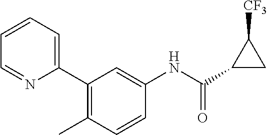 [SMILES: Cc1ccc(NC(=O)[C@H]2C[C@@H]2C(F)(F)F)cc1-c1ccccn1]

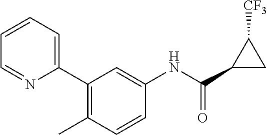 [SMILES: Cc1ccc(NC(=O)[C@@H]2C[C@H]2C(F)(F)F)cc1-c1ccccn1]

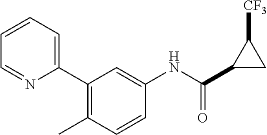 [SMILES: Cc1ccc(NC(=O)[C@@H]2C[C@@H]2C(F)(F)F)cc1-c1ccccn1]

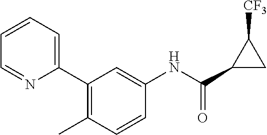 [SMILES: Cc1ccc(NC(=O)[C@@H]2C[C@@H]2C(F)(F)F)cc1-c1ccccn1]

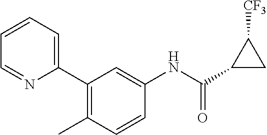 [SMILES: Cc1ccc(NC(=O)[C@H]2C[C@H]2C(F)(F)F)cc1-c1ccccn1]

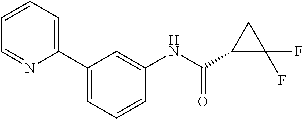 [SMILES: O=C(Nc1cccc(-c2ccccn2)c1)[C@@H]1CC1(F)F]

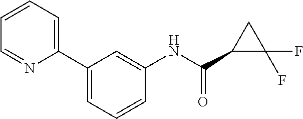 [SMILES: O=C(Nc1cccc(-c2ccccn2)c1)[C@H]1CC1(F)F]

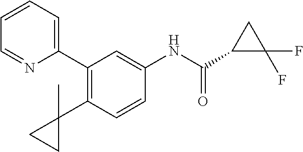 [SMILES: CC1(c2ccc(NC(=O)[C@@H]3CC3(F)F)cc2-c2ccccn2)CC1]

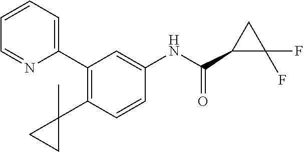 [SMILES: CC1(c2ccc(NC(=O)[C@H]3CC3(F)F)cc2-c2ccccn2)CC1]

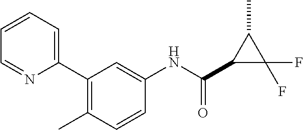

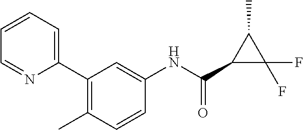 [SMILES: Cc1ccc(NC(=O)[C@H]2[C@H](C)C2(F)F)cc1-c1ccccn1]

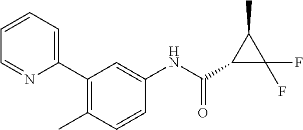 [SMILES: Cc1ccc(NC(=O)[C@@H]2[C@@H](C)C2(F)F)cc1-c1ccccn1]

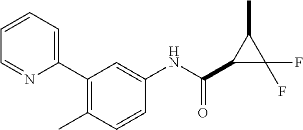

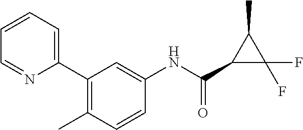 [SMILES: Cc1ccc(NC(=O)[C@H]2[C@@H](C)C2(F)F)cc1-c1ccccn1]

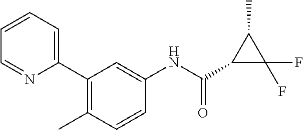 [SMILES: Cc1ccc(NC(=O)[C@@H]2[C@H](C)C2(F)F)cc1-c1ccccn1]

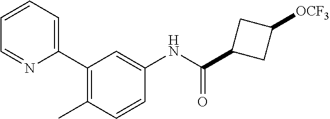

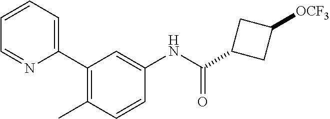

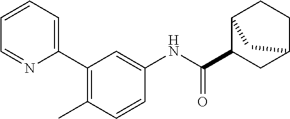

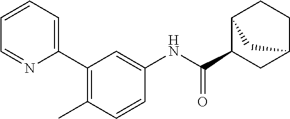 [SMILES: Cc1ccc(NC(=O)[C@@H]2C[C@@H]3CC[C@H]2C3)cc1-c1ccccn1]

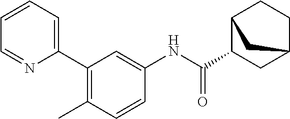 [SMILES: Cc1ccc(NC(=O)[C@H]2C[C@H]3CC[C@@H]2C3)cc1-c1ccccn1]

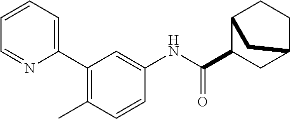

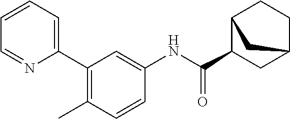 [SMILES: Cc1ccc(NC(=O)[C@@H]2C[C@H]3CC[C@@H]2C3)cc1-c1ccccn1]

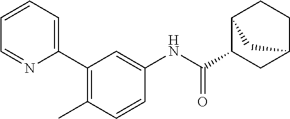 [SMILES: Cc1ccc(NC(=O)[C@H]2C[C@@H]3CC[C@H]2C3)cc1-c1ccccn1]

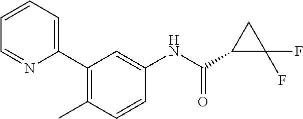 [SMILES: Cc1ccc(NC(=O)[C@@H]2CC2(F)F)cc1-c1ccccn1]

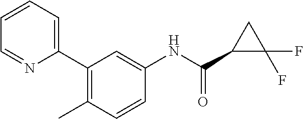 [SMILES: Cc1ccc(NC(=O)[C@H]2CC2(F)F)cc1-c1ccccn1]

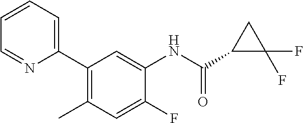 [SMILES: Cc1cc(F)c(NC(=O)[C@@H]2CC2(F)F)cc1-c1ccccn1]

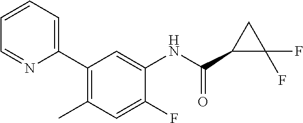 [SMILES: Cc1cc(F)c(NC(=O)[C@H]2CC2(F)F)cc1-c1ccccn1]

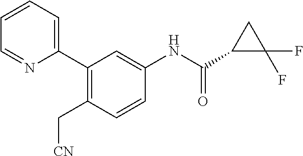 [SMILES: N#CCc1ccc(NC(=O)[C@@H]2CC2(F)F)cc1-c1ccccn1]

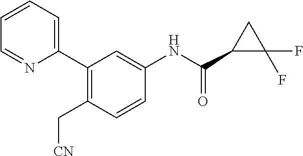 [SMILES: N#CCc1ccc(NC(=O)[C@H]2CC2(F)F)cc1-c1ccccn1]

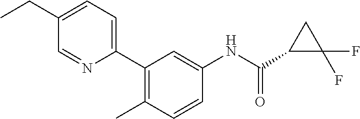 [SMILES: CCc1ccc(-c2cc(NC(=O)[C@@H]3CC3(F)F)ccc2C)nc1]

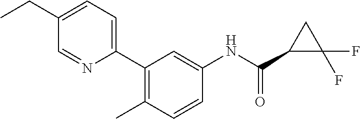 [SMILES: CCc1ccc(-c2cc(NC(=O)[C@H]3CC3(F)F)ccc2C)nc1]

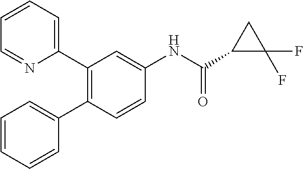 [SMILES: O=C(Nc1ccc(-c2ccccc2)c(-c2ccccn2)c1)[C@@H]1CC1(F)F]

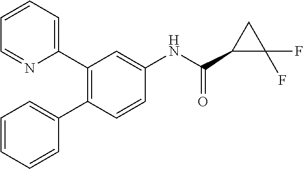 [SMILES: O=C(Nc1ccc(-c2ccccc2)c(-c2ccccn2)c1)[C@H]1CC1(F)F]

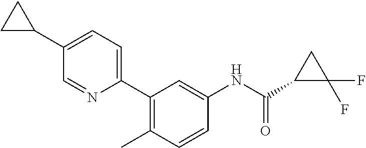 [SMILES: Cc1ccc(NC(=O)[C@@H]2CC2(F)F)cc1-c1ccc(C2CC2)cn1]

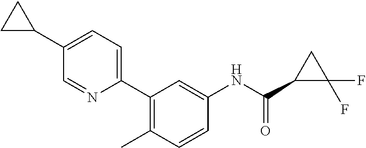 [SMILES: Cc1ccc(NC(=O)[C@H]2CC2(F)F)cc1-c1ccc(C2CC2)cn1]

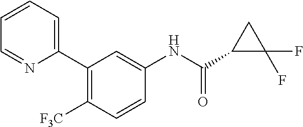 [SMILES: O=C(Nc1ccc(C(F)(F)F)c(-c2ccccn2)c1)[C@@H]1CC1(F)F]

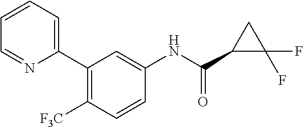 [SMILES: O=C(Nc1ccc(C(F)(F)F)c(-c2ccccn2)c1)[C@H]1CC1(F)F]

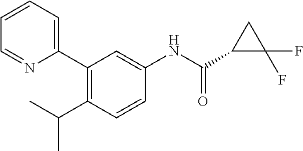 [SMILES: CC(C)c1ccc(NC(=O)[C@@H]2CC2(F)F)cc1-c1ccccn1]

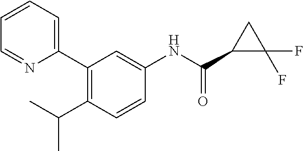 [SMILES: CC(C)c1ccc(NC(=O)[C@H]2CC2(F)F)cc1-c1ccccn1]

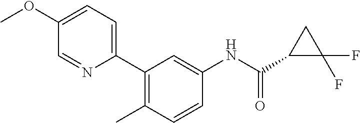 [SMILES: COc1ccc(-c2cc(NC(=O)[C@@H]3CC3(F)F)ccc2C)nc1]

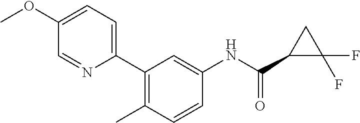 [SMILES: COc1ccc(-c2cc(NC(=O)[C@H]3CC3(F)F)ccc2C)nc1]

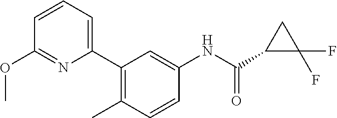 [SMILES: COc1cccc(-c2cc(NC(=O)[C@@H]3CC3(F)F)ccc2C)n1]

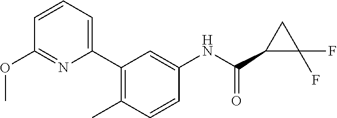 [SMILES: COc1cccc(-c2cc(NC(=O)[C@H]3CC3(F)F)ccc2C)n1]

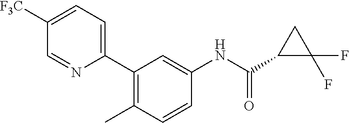 [SMILES: Cc1ccc(NC(=O)[C@@H]2CC2(F)F)cc1-c1ccc(C(F)(F)F)cn1]

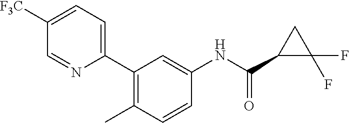 [SMILES: Cc1ccc(NC(=O)[C@H]2CC2(F)F)cc1-c1ccc(C(F)(F)F)cn1]

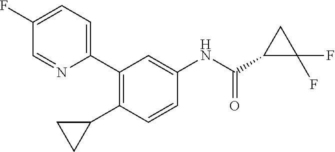 [SMILES: O=C(Nc1ccc(C2CC2)c(-c2ccc(F)cn2)c1)[C@@H]1CC1(F)F]

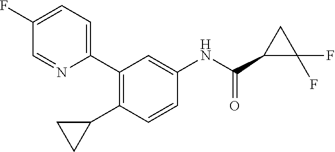 [SMILES: O=C(Nc1ccc(C2CC2)c(-c2ccc(F)cn2)c1)[C@H]1CC1(F)F]

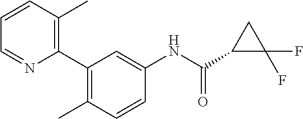 [SMILES: Cc1ccc(NC(=O)[C@@H]2CC2(F)F)cc1-c1ncccc1C]

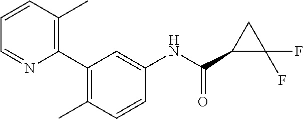 [SMILES: Cc1ccc(NC(=O)[C@H]2CC2(F)F)cc1-c1ncccc1C]

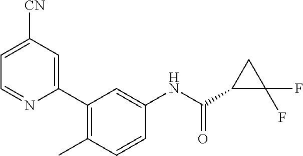 [SMILES: Cc1ccc(NC(=O)[C@@H]2CC2(F)F)cc1-c1cc(C#N)ccn1]

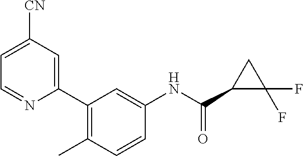 [SMILES: Cc1ccc(NC(=O)[C@H]2CC2(F)F)cc1-c1cc(C#N)ccn1]

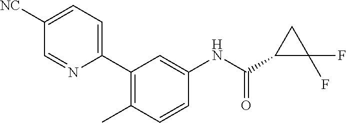 [SMILES: Cc1ccc(NC(=O)[C@@H]2CC2(F)F)cc1-c1ccc(C#N)cn1]

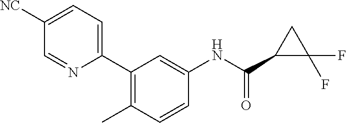 [SMILES: Cc1ccc(NC(=O)[C@H]2CC2(F)F)cc1-c1ccc(C#N)cn1]

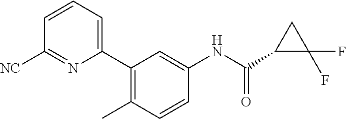 [SMILES: Cc1ccc(NC(=O)[C@@H]2CC2(F)F)cc1-c1cccc(C#N)n1]

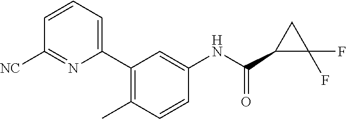 [SMILES: Cc1ccc(NC(=O)[C@H]2CC2(F)F)cc1-c1cccc(C#N)n1]

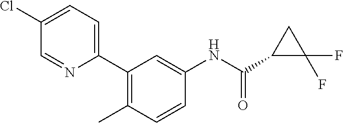 [SMILES: Cc1ccc(NC(=O)[C@@H]2CC2(F)F)cc1-c1ccc(Cl)cn1]

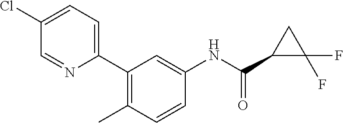 [SMILES: Cc1ccc(NC(=O)[C@H]2CC2(F)F)cc1-c1ccc(Cl)cn1]

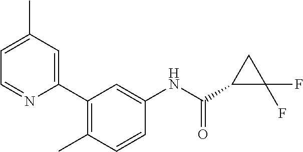 [SMILES: Cc1ccnc(-c2cc(NC(=O)[C@@H]3CC3(F)F)ccc2C)c1]

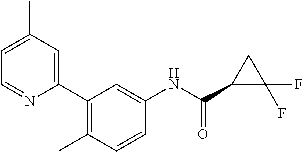 [SMILES: Cc1ccnc(-c2cc(NC(=O)[C@H]3CC3(F)F)ccc2C)c1]

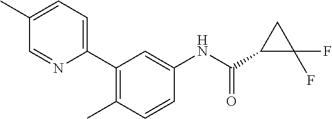 [SMILES: Cc1ccc(-c2cc(NC(=O)[C@@H]3CC3(F)F)ccc2C)nc1]

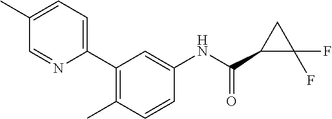 [SMILES: Cc1ccc(-c2cc(NC(=O)[C@H]3CC3(F)F)ccc2C)nc1]

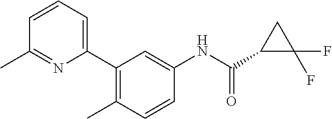 [SMILES: Cc1cccc(-c2cc(NC(=O)[C@@H]3CC3(F)F)ccc2C)n1]

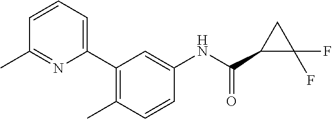 [SMILES: Cc1cccc(-c2cc(NC(=O)[C@H]3CC3(F)F)ccc2C)n1]

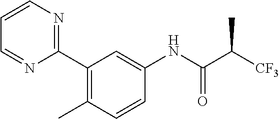 [SMILES: Cc1ccc(NC(=O)[C@@H](C)C(F)(F)F)cc1-c1ncccn1]

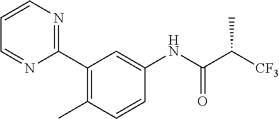 [SMILES: Cc1ccc(NC(=O)[C@H](C)C(F)(F)F)cc1-c1ncccn1]

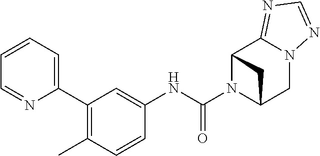 [SMILES: Cc1ccc(NC(=O)N2[C@@H]3C[C@H]2c2ncnn2C3)cc1-c1ccccn1]

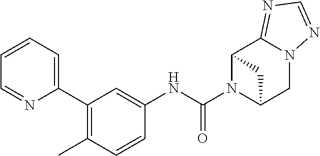 [SMILES: Cc1ccc(NC(=O)N2[C@H]3C[C@@H]2c2ncnn2C3)cc1-c1ccccn1]

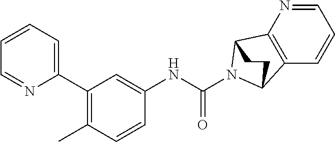 [SMILES: Cc1ccc(NC(=O)N2[C@@H]3CC[C@H]2c2ncccc23)cc1-c1ccccn1]

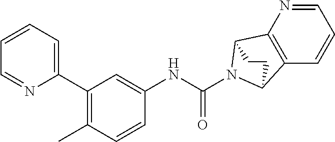 [SMILES: Cc1ccc(NC(=O)N2[C@@H]3CC[C@H]2c2cccnc23)cc1-c1ccccn1]

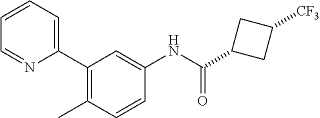

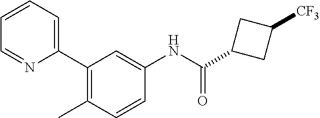

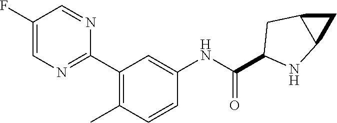 [SMILES: Cc1ccc(NC(=O)[C@H]2C[C@H]3C[C@H]3N2)cc1-c1ncc(F)cn1]

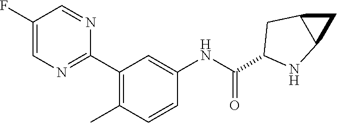 [SMILES: Cc1ccc(NC(=O)[C@@H]2C[C@H]3C[C@H]3N2)cc1-c1ncc(F)cn1]

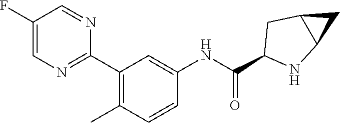 [SMILES: Cc1ccc(NC(=O)[C@H]2C[C@H]3C[C@H]3N2)cc1-c1ncc(F)cn1]

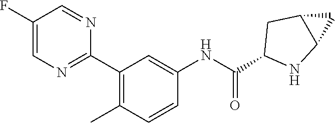 [SMILES: Cc1ccc(NC(=O)[C@@H]2C[C@@H]3C[C@@H]3N2)cc1-c1ncc(F)cn1]

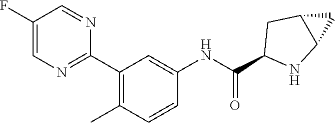 [SMILES: Cc1ccc(NC(=O)[C@H]2C[C@@H]3C[C@@H]3N2)cc1-c1ncc(F)cn1]

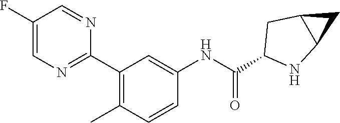 [SMILES: Cc1ccc(NC(=O)[C@@H]2C[C@H]3C[C@H]3N2)cc1-c1ncc(F)cn1]

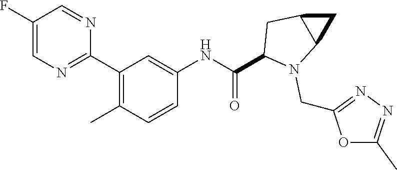 [SMILES: Cc1nnc(CN2[C@@H](C(=O)Nc3ccc(C)c(-c4ncc(F)cn4)c3)C[C@H]3C[C@H]32)o1]

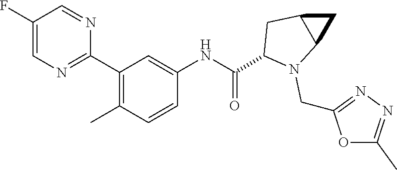 [SMILES: Cc1nnc(CN2[C@@H]3C[C@@H]3C[C@H]2C(=O)Nc2ccc(C)c(-c3ncc(F)cn3)c2)o1]

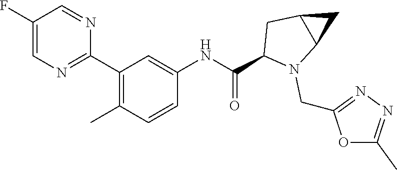 [SMILES: Cc1nnc(CN2[C@@H](C(=O)Nc3ccc(C)c(-c4ncc(F)cn4)c3)C[C@H]3C[C@H]32)o1]

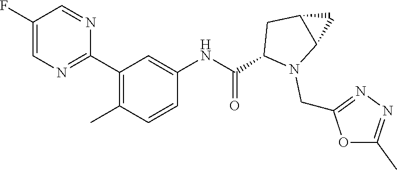 [SMILES: Cc1nnc(CN2[C@H](C(=O)Nc3ccc(C)c(-c4ncc(F)cn4)c3)C[C@@H]3C[C@@H]32)o1]

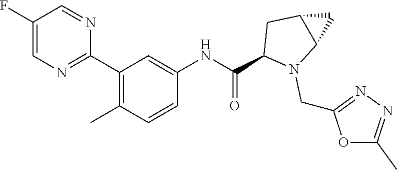 [SMILES: Cc1nnc(CN2[C@@H](C(=O)Nc3ccc(C)c(-c4ncc(F)cn4)c3)C[C@@H]3C[C@@H]32)o1]

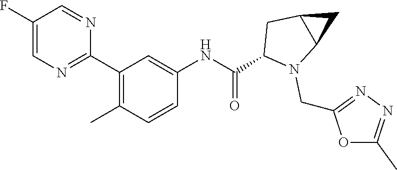 [SMILES: Cc1nnc(CN2[C@@H]3C[C@@H]3C[C@H]2C(=O)Nc2ccc(C)c(-c3ncc(F)cn3)c2)o1]

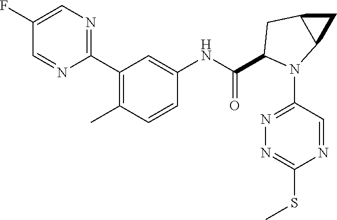 [SMILES: CSc1ncc(N2[C@@H](C(=O)Nc3ccc(C)c(-c4ncc(F)cn4)c3)C[C@H]3C[C@H]32)nn1]

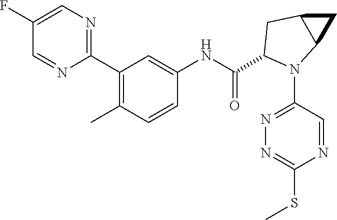 [SMILES: CSc1ncc(N2[C@@H]3C[C@@H]3C[C@H]2C(=O)Nc2ccc(C)c(-c3ncc(F)cn3)c2)nn1]

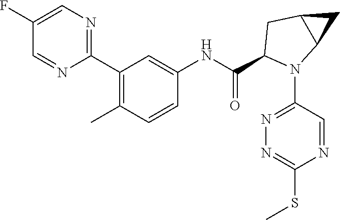 [SMILES: CSc1ncc(N2[C@@H](C(=O)Nc3ccc(C)c(-c4ncc(F)cn4)c3)C[C@H]3C[C@H]32)nn1]

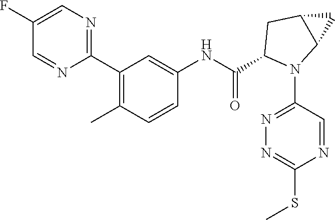 [SMILES: CSc1ncc(N2[C@H](C(=O)Nc3ccc(C)c(-c4ncc(F)cn4)c3)C[C@@H]3C[C@@H]32)nn1]

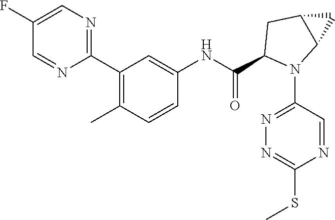 [SMILES: CSc1ncc(N2[C@@H](C(=O)Nc3ccc(C)c(-c4ncc(F)cn4)c3)C[C@@H]3C[C@@H]32)nn1]

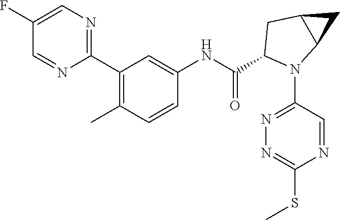 [SMILES: CSc1ncc(N2[C@@H]3C[C@@H]3C[C@H]2C(=O)Nc2ccc(C)c(-c3ncc(F)cn3)c2)nn1]

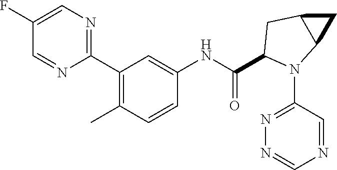 [SMILES: Cc1ccc(NC(=O)[C@H]2C[C@H]3C[C@H]3N2c2cncnn2)cc1-c1ncc(F)cn1]

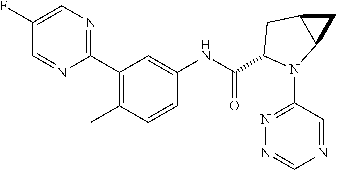 [SMILES: Cc1ccc(NC(=O)[C@@H]2C[C@H]3C[C@H]3N2c2cncnn2)cc1-c1ncc(F)cn1]

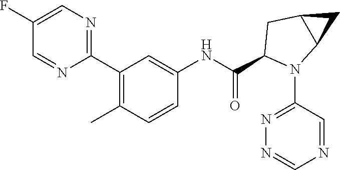 [SMILES: Cc1ccc(NC(=O)[C@H]2C[C@H]3C[C@H]3N2c2cncnn2)cc1-c1ncc(F)cn1]

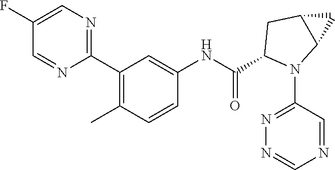 [SMILES: Cc1ccc(NC(=O)[C@@H]2C[C@@H]3C[C@@H]3N2c2cncnn2)cc1-c1ncc(F)cn1]

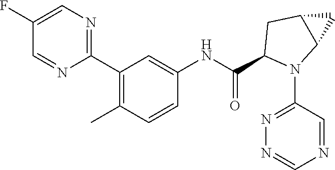 [SMILES: Cc1ccc(NC(=O)[C@H]2C[C@@H]3C[C@@H]3N2c2cncnn2)cc1-c1ncc(F)cn1]

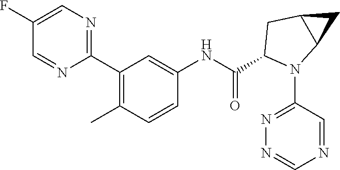 [SMILES: Cc1ccc(NC(=O)[C@@H]2C[C@H]3C[C@H]3N2c2cncnn2)cc1-c1ncc(F)cn1]

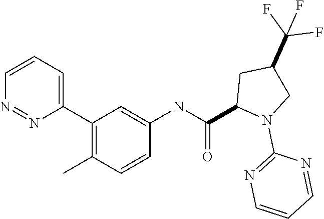

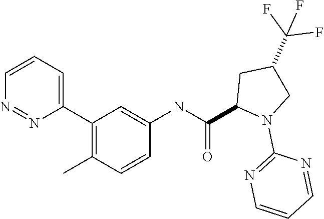

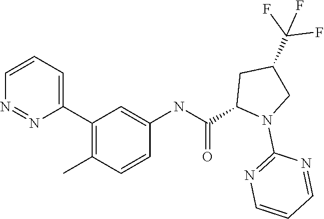

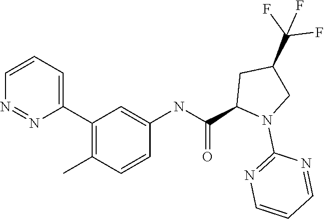

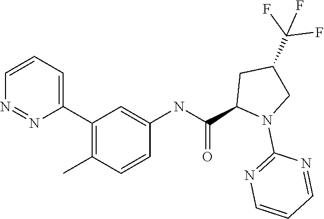

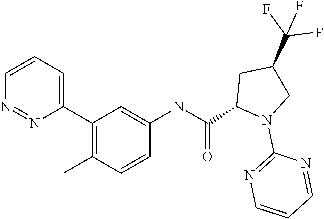

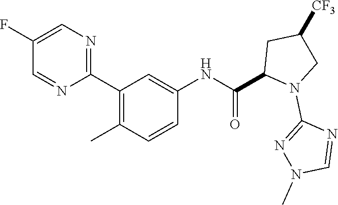 [SMILES: Cc1ccc(NC(=O)[C@H]2C[C@@H](C(F)(F)F)CN2c2ncn(C)n2)cc1-c1ncc(F)cn1]

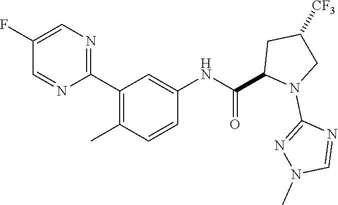 [SMILES: Cc1ccc(NC(=O)[C@H]2C[C@H](C(F)(F)F)CN2c2ncn(C)n2)cc1-c1ncc(F)cn1]

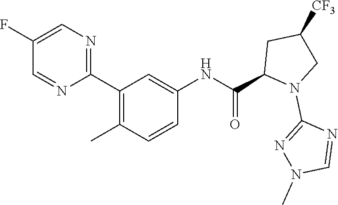 [SMILES: Cc1ccc(NC(=O)[C@H]2C[C@@H](C(F)(F)F)CN2c2ncn(C)n2)cc1-c1ncc(F)cn1]

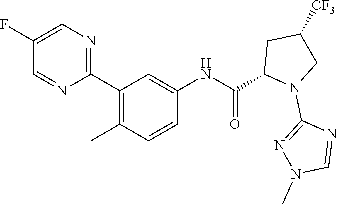 [SMILES: Cc1ccc(NC(=O)[C@@H]2C[C@H](C(F)(F)F)CN2c2ncn(C)n2)cc1-c1ncc(F)cn1]

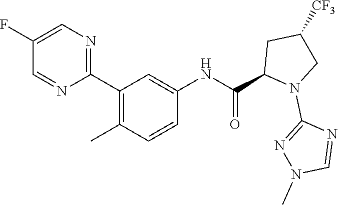 [SMILES: Cc1ccc(NC(=O)[C@H]2C[C@H](C(F)(F)F)CN2c2ncn(C)n2)cc1-c1ncc(F)cn1]

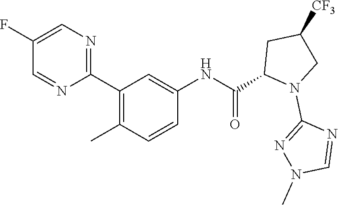 [SMILES: Cc1ccc(NC(=O)[C@@H]2C[C@@H](C(F)(F)F)CN2c2ncn(C)n2)cc1-c1ncc(F)cn1]

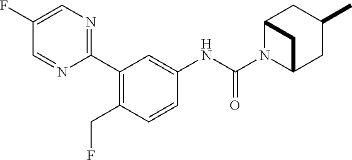

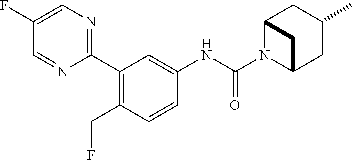

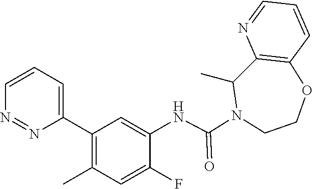 [SMILES: Cc1cc(F)c(NC(=O)N2CCOc3cccnc3C2C)cc1-c1cccnn1]

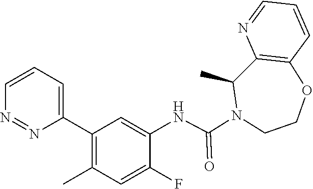 [SMILES: Cc1cc(F)c(NC(=O)N2CCOc3cccnc3[C@@H]2C)cc1-c1cccnn1]

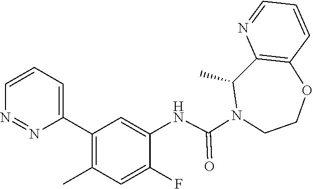 [SMILES: Cc1cc(F)c(NC(=O)N2CCOc3cccnc3[C@H]2C)cc1-c1cccnn1]

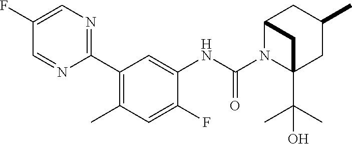

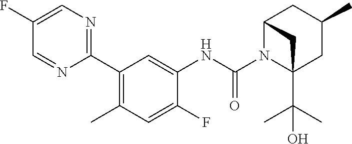 [SMILES: Cc1cc(F)c(NC(=O)N2[C@@H]3C[C@@H](C)C[C@@]2(C(C)(C)O)C3)cc1-c1ncc(F)cn1]

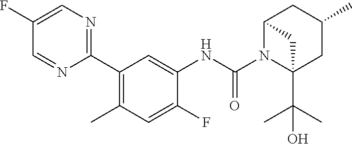 [SMILES: Cc1cc(F)c(NC(=O)N2[C@H]3C[C@H](C)C[C@]2(C(C)(C)O)C3)cc1-c1ncc(F)cn1]

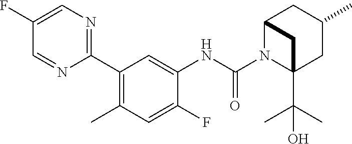

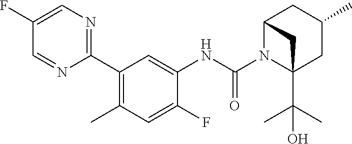 [SMILES: Cc1cc(F)c(NC(=O)N2[C@@H]3C[C@H](C)C[C@@]2(C(C)(C)O)C3)cc1-c1ncc(F)cn1]

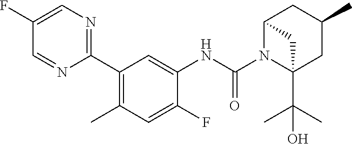 [SMILES: Cc1cc(F)c(NC(=O)N2[C@H]3C[C@@H](C)C[C@]2(C(C)(C)O)C3)cc1-c1ncc(F)cn1]

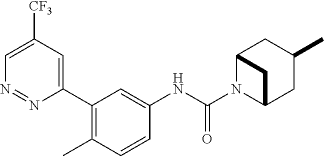

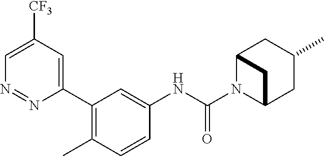

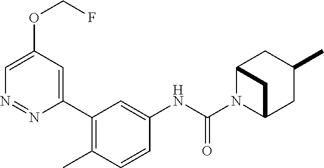

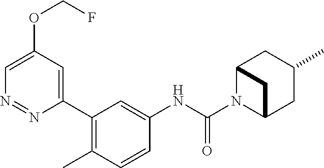

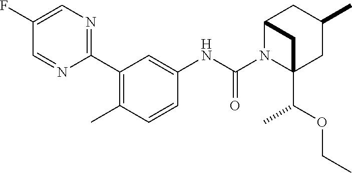

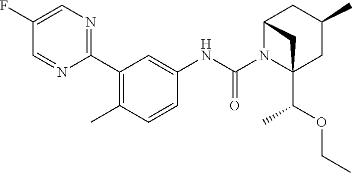 [SMILES: CCO[C@H](C)[C@]12C[C@H](C)C[C@H](C1)N2C(=O)Nc1ccc(C)c(-c2ncc(F)cn2)c1]

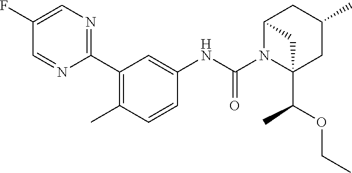 [SMILES: CCO[C@@H](C)[C@@]12C[C@@H](C)C[C@@H](C1)N2C(=O)Nc1ccc(C)c(-c2ncc(F)cn2)c1]

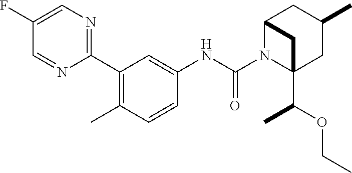

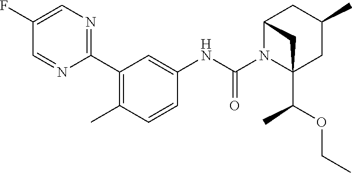 [SMILES: CCO[C@@H](C)[C@]12C[C@H](C)C[C@H](C1)N2C(=O)Nc1ccc(C)c(-c2ncc(F)cn2)c1]

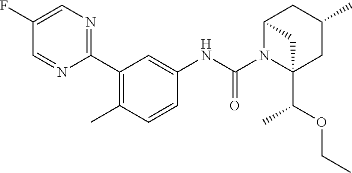 [SMILES: CCO[C@H](C)[C@@]12C[C@@H](C)C[C@@H](C1)N2C(=O)Nc1ccc(C)c(-c2ncc(F)cn2)c1]

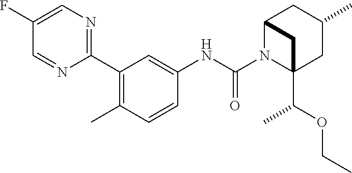

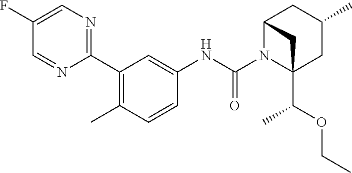 [SMILES: CCO[C@H](C)[C@]12C[C@@H](C)C[C@H](C1)N2C(=O)Nc1ccc(C)c(-c2ncc(F)cn2)c1]

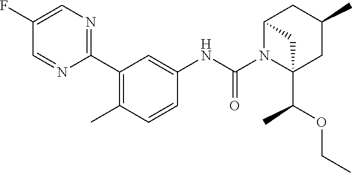 [SMILES: CCO[C@@H](C)[C@@]12C[C@H](C)C[C@@H](C1)N2C(=O)Nc1ccc(C)c(-c2ncc(F)cn2)c1]

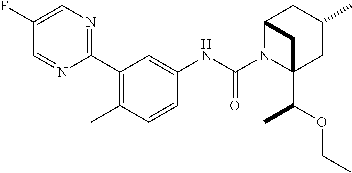

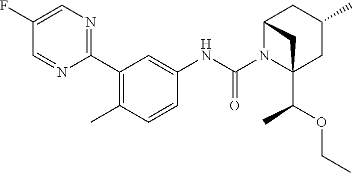 [SMILES: CCO[C@@H](C)[C@]12C[C@@H](C)C[C@H](C1)N2C(=O)Nc1ccc(C)c(-c2ncc(F)cn2)c1]

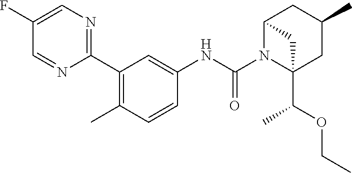 [SMILES: CCO[C@H](C)[C@@]12C[C@H](C)C[C@@H](C1)N2C(=O)Nc1ccc(C)c(-c2ncc(F)cn2)c1]

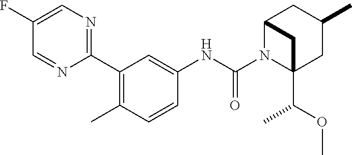

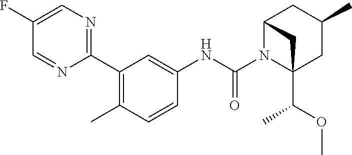 [SMILES: CO[C@H](C)[C@]12C[C@H](C)C[C@H](C1)N2C(=O)Nc1ccc(C)c(-c2ncc(F)cn2)c1]

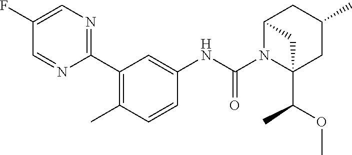 [SMILES: CO[C@@H](C)[C@@]12C[C@@H](C)C[C@@H](C1)N2C(=O)Nc1ccc(C)c(-c2ncc(F)cn2)c1]

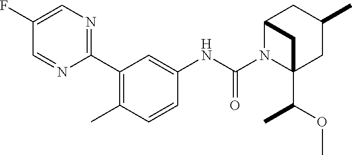

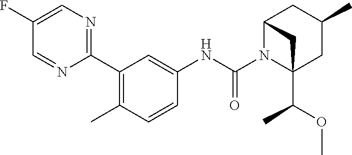 [SMILES: CO[C@@H](C)[C@]12C[C@H](C)C[C@H](C1)N2C(=O)Nc1ccc(C)c(-c2ncc(F)cn2)c1]

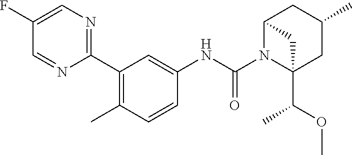 [SMILES: CO[C@H](C)[C@@]12C[C@@H](C)C[C@@H](C1)N2C(=O)Nc1ccc(C)c(-c2ncc(F)cn2)c1]

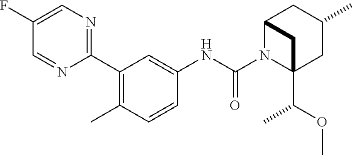

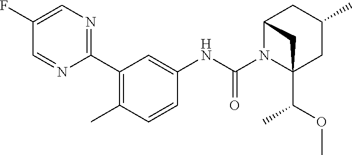 [SMILES: CO[C@H](C)[C@]12C[C@@H](C)C[C@H](C1)N2C(=O)Nc1ccc(C)c(-c2ncc(F)cn2)c1]

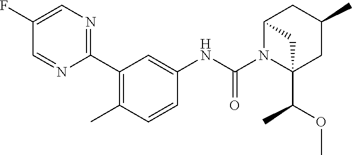 [SMILES: CO[C@@H](C)[C@@]12C[C@H](C)C[C@@H](C1)N2C(=O)Nc1ccc(C)c(-c2ncc(F)cn2)c1]

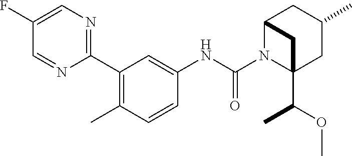

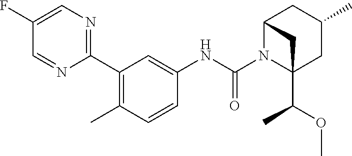 [SMILES: CO[C@@H](C)[C@]12C[C@@H](C)C[C@H](C1)N2C(=O)Nc1ccc(C)c(-c2ncc(F)cn2)c1]

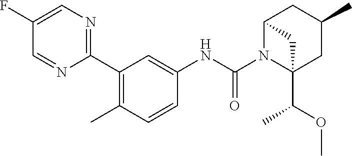 [SMILES: CO[C@H](C)[C@@]12C[C@H](C)C[C@@H](C1)N2C(=O)Nc1ccc(C)c(-c2ncc(F)cn2)c1]

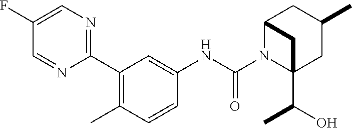

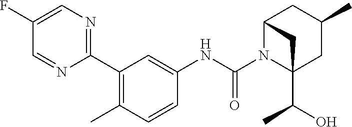 [SMILES: Cc1ccc(NC(=O)N2[C@@H]3C[C@@H](C)C[C@@]2([C@H](C)O)C3)cc1-c1ncc(F)cn1]

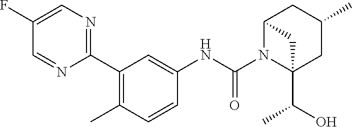 [SMILES: Cc1ccc(NC(=O)N2[C@H]3C[C@H](C)C[C@]2([C@@H](C)O)C3)cc1-c1ncc(F)cn1]

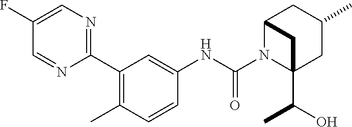

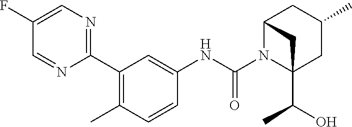 [SMILES: Cc1ccc(NC(=O)N2[C@@H]3C[C@H](C)C[C@@]2([C@H](C)O)C3)cc1-c1ncc(F)cn1]

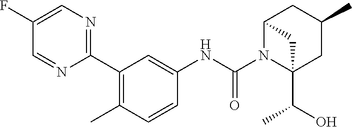 [SMILES: Cc1ccc(NC(=O)N2[C@H]3C[C@@H](C)C[C@]2([C@@H](C)O)C3)cc1-c1ncc(F)cn1]

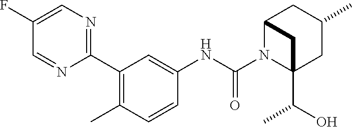

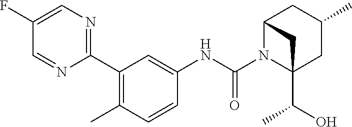 [SMILES: Cc1ccc(NC(=O)N2[C@@H]3C[C@H](C)C[C@@]2([C@@H](C)O)C3)cc1-c1ncc(F)cn1]

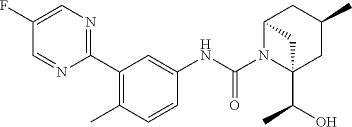 [SMILES: Cc1ccc(NC(=O)N2[C@H]3C[C@@H](C)C[C@]2([C@H](C)O)C3)cc1-c1ncc(F)cn1]

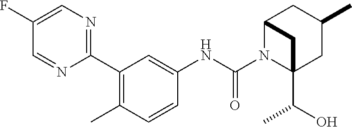

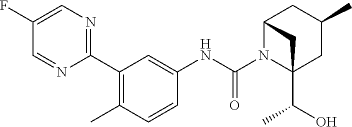 [SMILES: Cc1ccc(NC(=O)N2[C@@H]3C[C@@H](C)C[C@@]2([C@@H](C)O)C3)cc1-c1ncc(F)cn1]

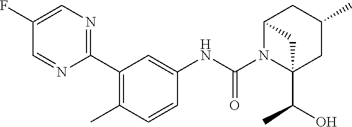 [SMILES: Cc1ccc(NC(=O)N2[C@H]3C[C@H](C)C[C@]2([C@H](C)O)C3)cc1-c1ncc(F)cn1]

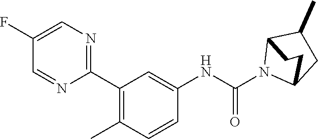 [SMILES: Cc1ccc(NC(=O)N2[C@@H]3CC[C@H]2[C@@H](C)C3)cc1-c1ncc(F)cn1]

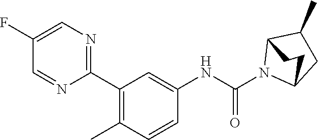 [SMILES: Cc1ccc(NC(=O)N2[C@@H]3CC[C@H]2[C@@H](C)C3)cc1-c1ncc(F)cn1]

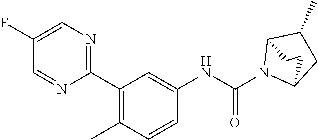 [SMILES: Cc1ccc(NC(=O)N2[C@H]3CC[C@@H]2[C@H](C)C3)cc1-c1ncc(F)cn1]

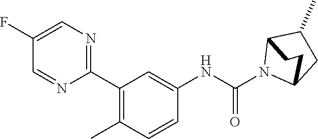 [SMILES: Cc1ccc(NC(=O)N2[C@@H]3CC[C@H]2[C@H](C)C3)cc1-c1ncc(F)cn1]

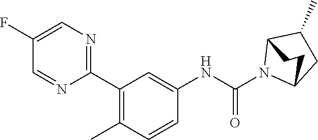 [SMILES: Cc1ccc(NC(=O)N2[C@@H]3CC[C@H]2[C@H](C)C3)cc1-c1ncc(F)cn1]

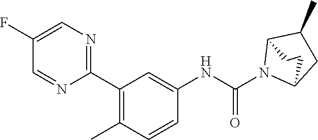 [SMILES: Cc1ccc(NC(=O)N2[C@H]3CC[C@@H]2[C@@H](C)C3)cc1-c1ncc(F)cn1]

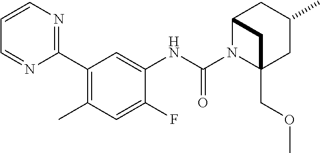

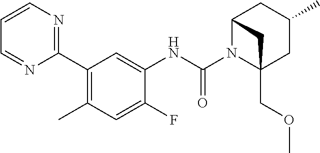 [SMILES: COC[C@]12C[C@@H](C)C[C@H](C1)N2C(=O)Nc1cc(-c2ncccn2)c(C)cc1F]

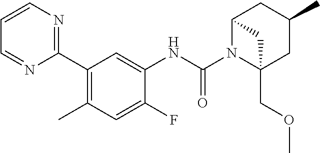 [SMILES: COC[C@@]12C[C@H](C)C[C@@H](C1)N2C(=O)Nc1cc(-c2ncccn2)c(C)cc1F]

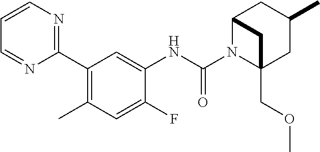

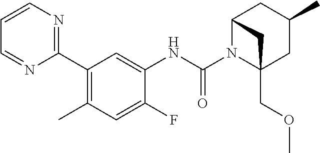 [SMILES: COC[C@]12C[C@H](C)C[C@H](C1)N2C(=O)Nc1cc(-c2ncccn2)c(C)cc1F]

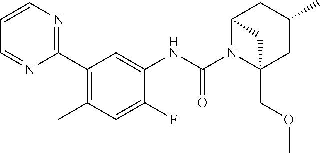 [SMILES: COC[C@@]12C[C@@H](C)C[C@@H](C1)N2C(=O)Nc1cc(-c2ncccn2)c(C)cc1F]

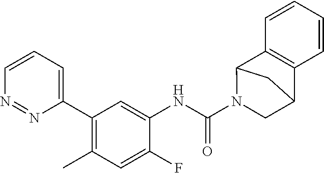 [SMILES: Cc1cc(F)c(NC(=O)N2CC3CC2c2ccccc23)cc1-c1cccnn1]

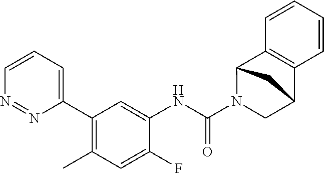 [SMILES: Cc1cc(F)c(NC(=O)N2C[C@@H]3C[C@H]2c2ccccc23)cc1-c1cccnn1]

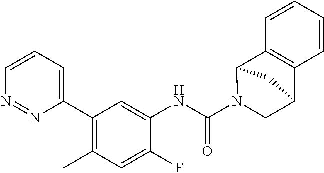 [SMILES: Cc1cc(F)c(NC(=O)N2C[C@H]3C[C@@H]2c2ccccc23)cc1-c1cccnn1]

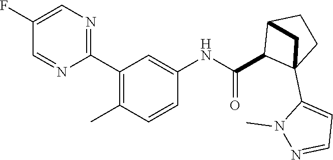

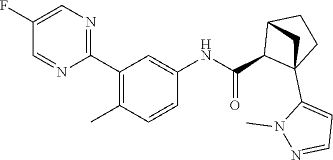 [SMILES: Cc1ccc(NC(=O)[C@H]2[C@@H]3CC[C@@]2(c2ccnn2C)C3)cc1-c1ncc(F)cn1]

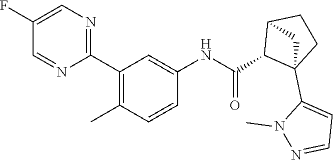 [SMILES: Cc1ccc(NC(=O)[C@@H]2[C@H]3CC[C@]2(c2ccnn2C)C3)cc1-c1ncc(F)cn1]

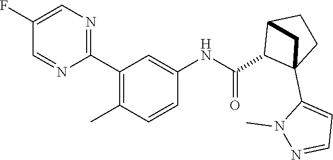

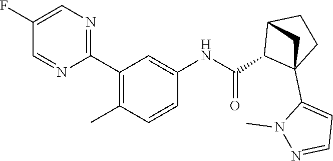 [SMILES: Cc1ccc(NC(=O)[C@@H]2[C@@H]3CC[C@@]2(c2ccnn2C)C3)cc1-c1ncc(F)cn1]

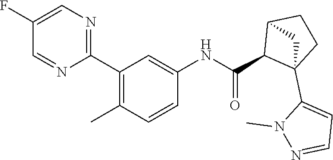 [SMILES: Cc1ccc(NC(=O)[C@H]2[C@H]3CC[C@]2(c2ccnn2C)C3)cc1-c1ncc(F)cn1]

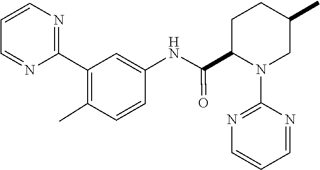 [SMILES: Cc1ccc(NC(=O)[C@H]2CC[C@@H](C)CN2c2ncccn2)cc1-c1ncccn1]

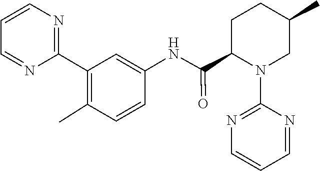 [SMILES: Cc1ccc(NC(=O)[C@H]2CC[C@@H](C)CN2c2ncccn2)cc1-c1ncccn1]

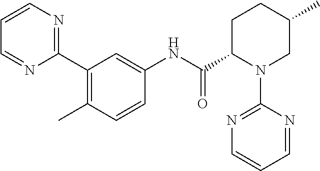 [SMILES: Cc1ccc(NC(=O)[C@@H]2CC[C@H](C)CN2c2ncccn2)cc1-c1ncccn1]

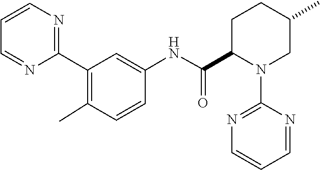 [SMILES: Cc1ccc(NC(=O)[C@H]2CC[C@H](C)CN2c2ncccn2)cc1-c1ncccn1]

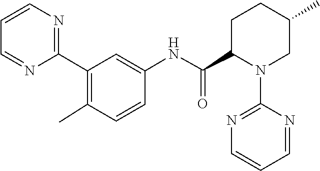

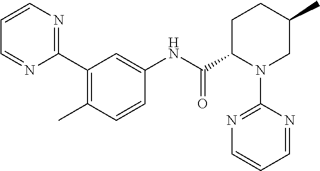 [SMILES: Cc1ccc(NC(=O)[C@@H]2CC[C@@H](C)CN2c2ncccn2)cc1-c1ncccn1]

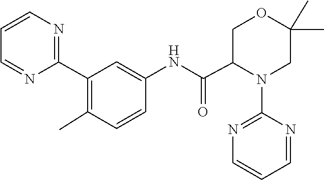 [SMILES: Cc1ccc(NC(=O)C2COC(C)(C)CN2c2ncccn2)cc1-c1ncccn1]

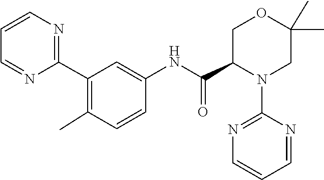 [SMILES: Cc1ccc(NC(=O)[C@H]2COC(C)(C)CN2c2ncccn2)cc1-c1ncccn1]

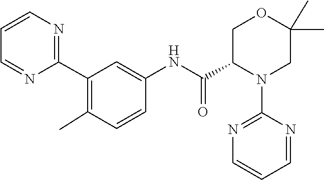 [SMILES: Cc1ccc(NC(=O)[C@@H]2COC(C)(C)CN2c2ncccn2)cc1-c1ncccn1]

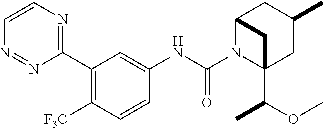

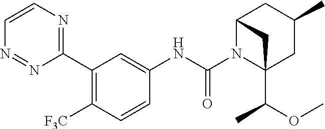 [SMILES: CO[C@@H](C)[C@]12C[C@H](C)C[C@H](C1)N2C(=O)Nc1ccc(C(F)(F)F)c(-c2nccnn2)c1]

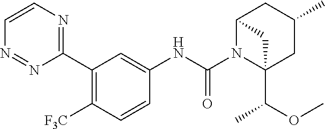 [SMILES: CO[C@H](C)[C@@]12C[C@@H](C)C[C@@H](C1)N2C(=O)Nc1ccc(C(F)(F)F)c(-c2nccnn2)c1]

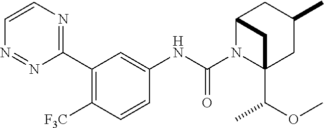

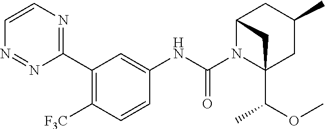 [SMILES: CO[C@H](C)[C@]12C[C@H](C)C[C@H](C1)N2C(=O)Nc1ccc(C(F)(F)F)c(-c2nccnn2)c1]

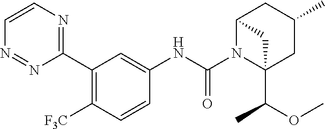 [SMILES: CO[C@@H](C)[C@@]12C[C@@H](C)C[C@@H](C1)N2C(=O)Nc1ccc(C(F)(F)F)c(-c2nccnn2)c1]

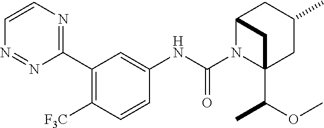

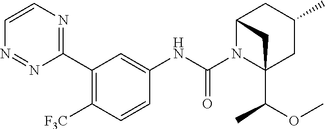 [SMILES: CO[C@@H](C)[C@]12C[C@@H](C)C[C@H](C1)N2C(=O)Nc1ccc(C(F)(F)F)c(-c2nccnn2)c1]

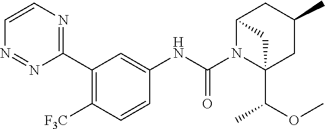 [SMILES: CO[C@H](C)[C@@]12C[C@H](C)C[C@@H](C1)N2C(=O)Nc1ccc(C(F)(F)F)c(-c2nccnn2)c1]

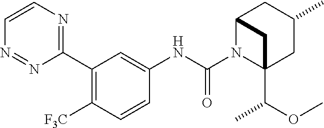

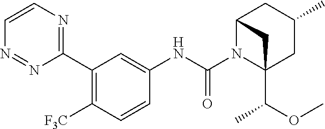 [SMILES: CO[C@H](C)[C@]12C[C@@H](C)C[C@H](C1)N2C(=O)Nc1ccc(C(F)(F)F)c(-c2nccnn2)c1]

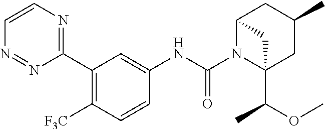 [SMILES: CO[C@@H](C)[C@@]12C[C@H](C)C[C@@H](C1)N2C(=O)Nc1ccc(C(F)(F)F)c(-c2nccnn2)c1]

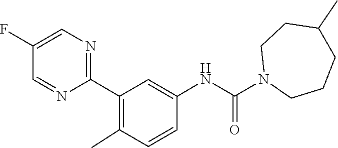 [SMILES: Cc1ccc(NC(=O)N2CCCC(C)CC2)cc1-c1ncc(F)cn1]

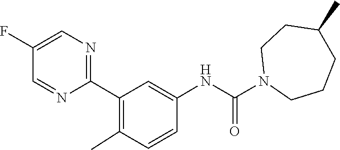 [SMILES: Cc1ccc(NC(=O)N2CCC[C@H](C)CC2)cc1-c1ncc(F)cn1]

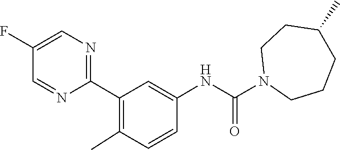 [SMILES: Cc1ccc(NC(=O)N2CCC[C@@H](C)CC2)cc1-c1ncc(F)cn1]

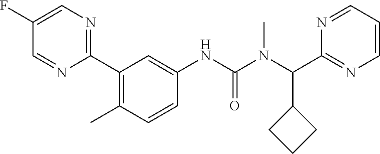 [SMILES: Cc1ccc(NC(=O)N(C)C(c2ncccn2)C2CCC2)cc1-c1ncc(F)cn1]

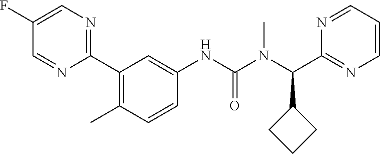 [SMILES: Cc1ccc(NC(=O)N(C)[C@@H](c2ncccn2)C2CCC2)cc1-c1ncc(F)cn1]

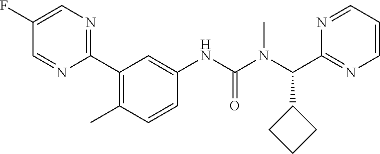 [SMILES: Cc1ccc(NC(=O)N(C)[C@H](c2ncccn2)C2CCC2)cc1-c1ncc(F)cn1]

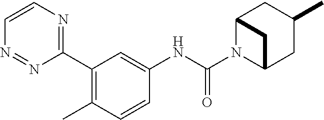

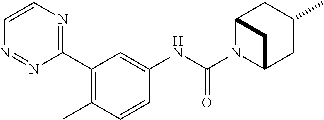

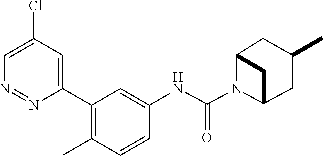

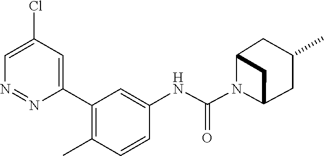

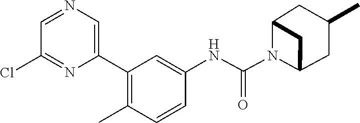

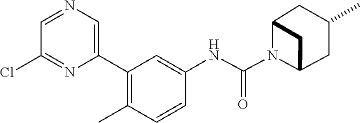

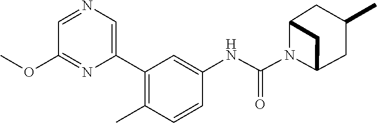

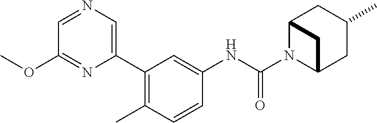

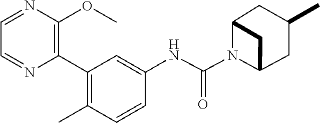

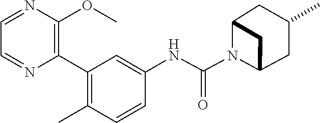

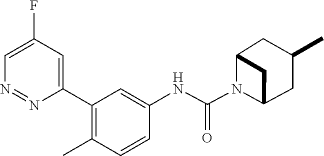

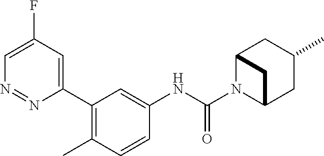

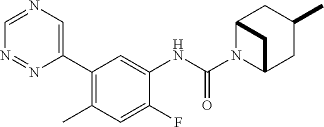

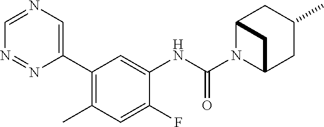

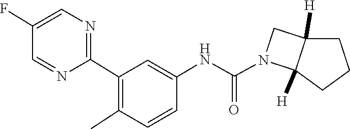 [SMILES: Cc1ccc(NC(=O)N2C[C@@H]3CCC[C@@H]32)cc1-c1ncc(F)cn1]

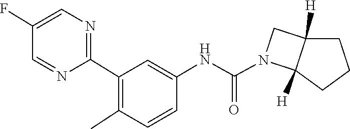 [SMILES: Cc1ccc(NC(=O)N2C[C@@H]3CCC[C@@H]32)cc1-c1ncc(F)cn1]

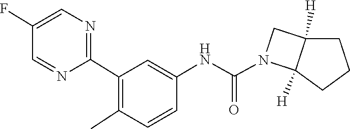 [SMILES: Cc1ccc(NC(=O)N2C[C@H]3CCC[C@H]32)cc1-c1ncc(F)cn1]

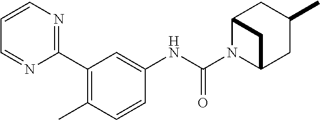

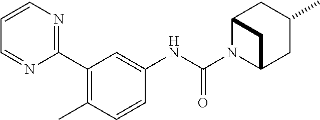

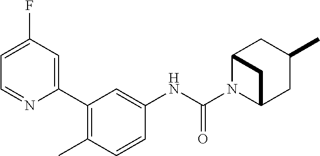

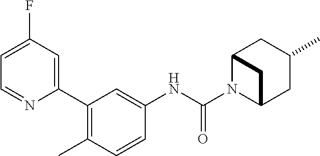

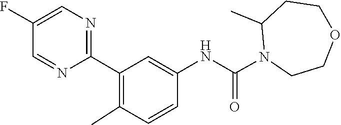 [SMILES: Cc1ccc(NC(=O)N2CCOCCC2C)cc1-c1ncc(F)cn1]

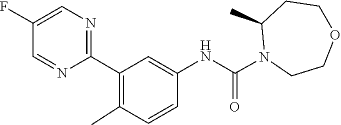 [SMILES: Cc1ccc(NC(=O)N2CCOCC[C@@H]2C)cc1-c1ncc(F)cn1]

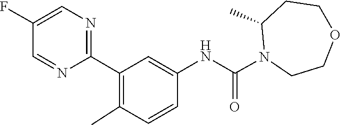 [SMILES: Cc1ccc(NC(=O)N2CCOCC[C@H]2C)cc1-c1ncc(F)cn1]

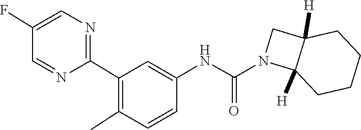 [SMILES: Cc1ccc(NC(=O)N2C[C@@H]3CCCC[C@@H]32)cc1-c1ncc(F)cn1]

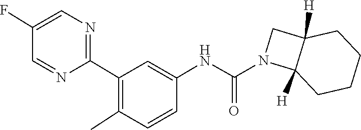 [SMILES: Cc1ccc(NC(=O)N2C[C@@H]3CCCC[C@@H]32)cc1-c1ncc(F)cn1]

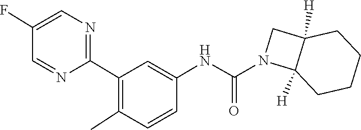 [SMILES: Cc1ccc(NC(=O)N2C[C@H]3CCCC[C@H]32)cc1-c1ncc(F)cn1]

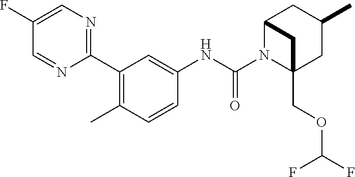

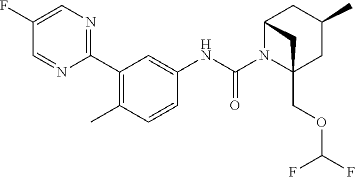 [SMILES: Cc1ccc(NC(=O)N2[C@@H]3C[C@@H](C)C[C@@]2(COC(F)F)C3)cc1-c1ncc(F)cn1]

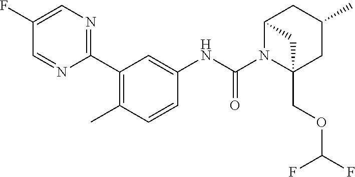 [SMILES: Cc1ccc(NC(=O)N2[C@H]3C[C@H](C)C[C@]2(COC(F)F)C3)cc1-c1ncc(F)cn1]

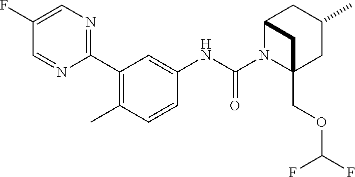

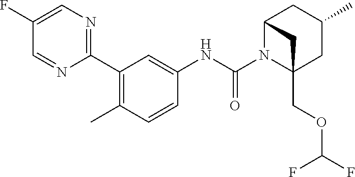 [SMILES: Cc1ccc(NC(=O)N2[C@@H]3C[C@H](C)C[C@@]2(COC(F)F)C3)cc1-c1ncc(F)cn1]

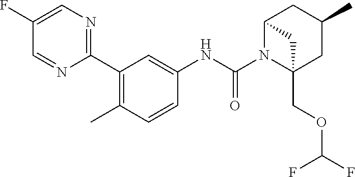 [SMILES: Cc1ccc(NC(=O)N2[C@H]3C[C@@H](C)C[C@]2(COC(F)F)C3)cc1-c1ncc(F)cn1]

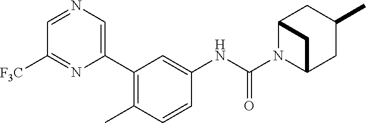

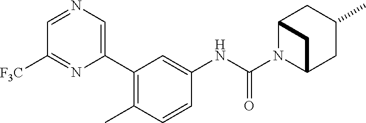

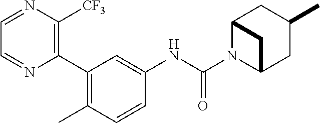

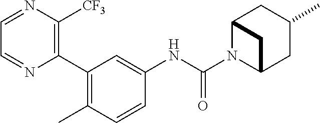

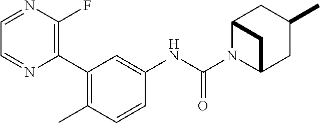

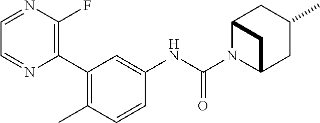

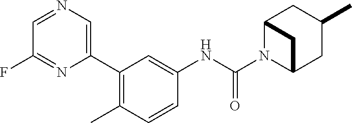

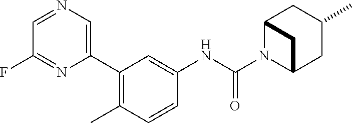

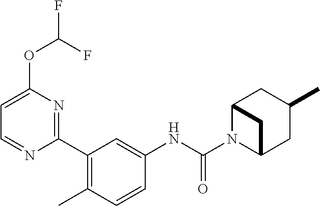

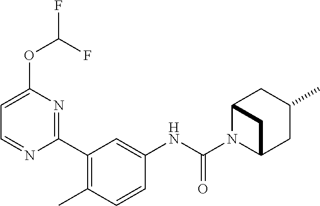

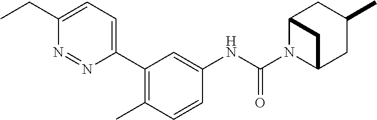

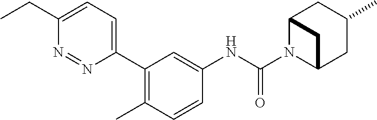

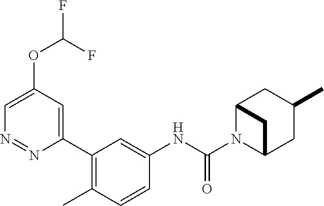

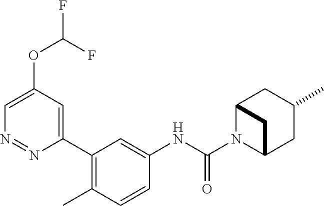

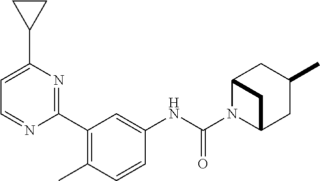

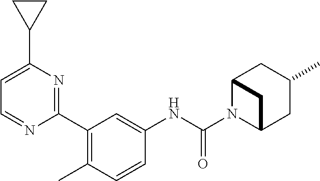

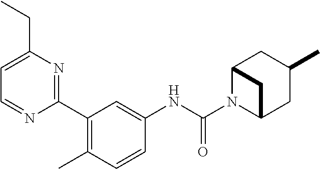

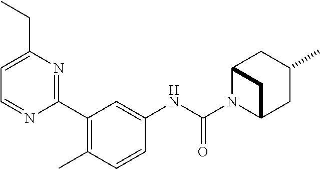

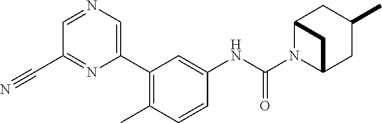

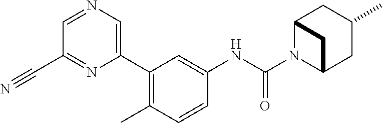

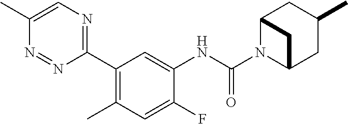

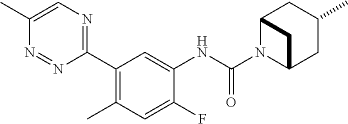

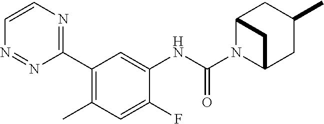 [SMILES: Cc1cc(F)c(NC(=O)N2[C@@H]3C[C@@H](C)C[C@H]2C3)cc1-c1nccnn1]

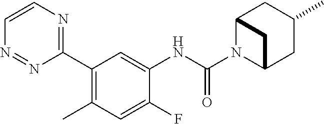 [SMILES: Cc1cc(F)c(NC(=O)N2[C@@H]3C[C@H](C)C[C@H]2C3)cc1-c1nccnn1]

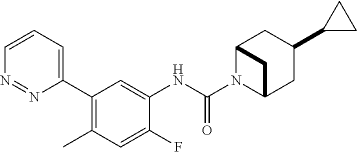

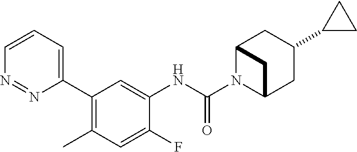

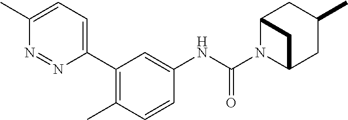

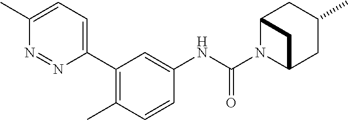

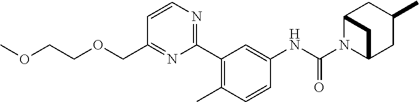

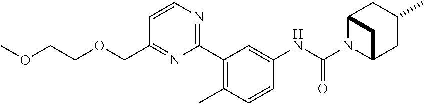

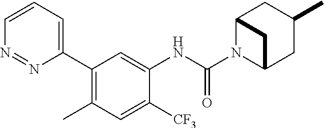

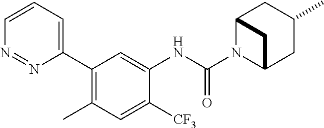

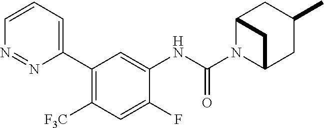

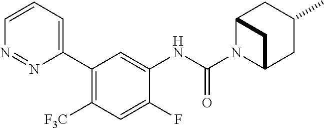

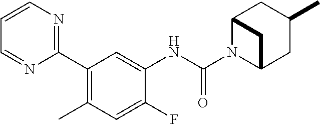

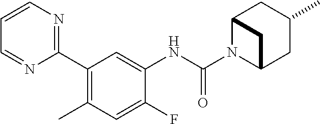

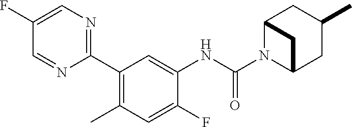

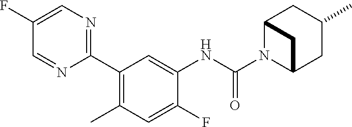

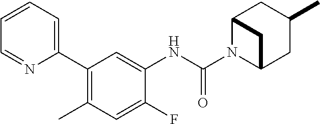

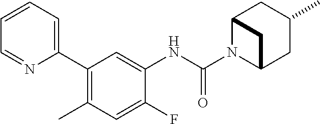

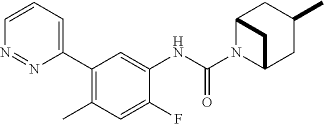 [SMILES: Cc1cc(F)c(NC(=O)N2[C@@H]3C[C@@H](C)C[C@H]2C3)cc1-c1cccnn1]

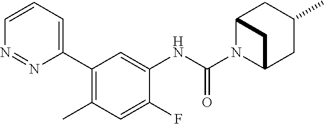 [SMILES: Cc1cc(F)c(NC(=O)N2[C@@H]3C[C@H](C)C[C@H]2C3)cc1-c1cccnn1]

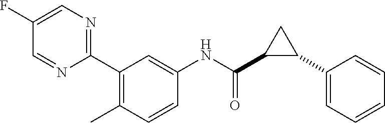 [SMILES: Cc1ccc(NC(=O)[C@H]2C[C@@H]2c2ccccc2)cc1-c1ncc(F)cn1]

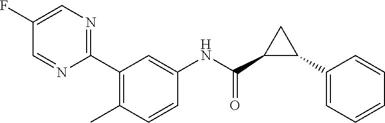 [SMILES: Cc1ccc(NC(=O)[C@H]2C[C@@H]2c2ccccc2)cc1-c1ncc(F)cn1]

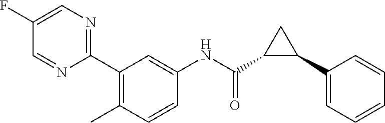 [SMILES: Cc1ccc(NC(=O)[C@@H]2C[C@H]2c2ccccc2)cc1-c1ncc(F)cn1]

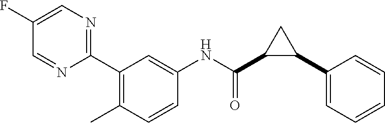 [SMILES: Cc1ccc(NC(=O)[C@H]2C[C@H]2c2ccccc2)cc1-c1ncc(F)cn1]

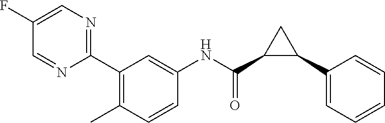 [SMILES: Cc1ccc(NC(=O)[C@H]2C[C@H]2c2ccccc2)cc1-c1ncc(F)cn1]

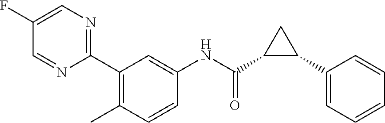 [SMILES: Cc1ccc(NC(=O)[C@@H]2C[C@@H]2c2ccccc2)cc1-c1ncc(F)cn1]

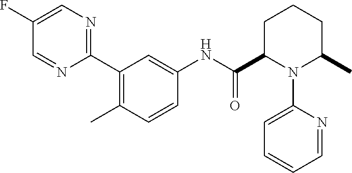

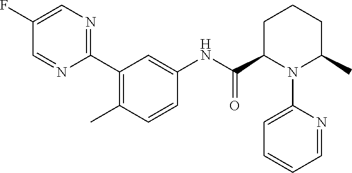 [SMILES: Cc1ccc(NC(=O)[C@H]2CCC[C@@H](C)N2c2ccccn2)cc1-c1ncc(F)cn1]

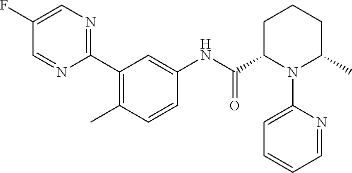 [SMILES: Cc1ccc(NC(=O)[C@@H]2CCC[C@H](C)N2c2ccccn2)cc1-c1ncc(F)cn1]

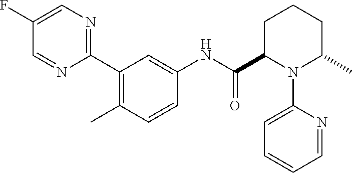

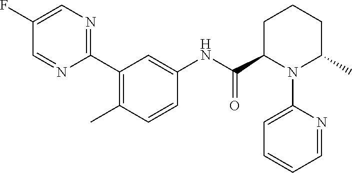 [SMILES: Cc1ccc(NC(=O)[C@H]2CCC[C@H](C)N2c2ccccn2)cc1-c1ncc(F)cn1]

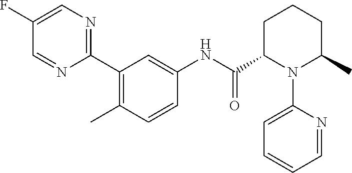 [SMILES: Cc1ccc(NC(=O)[C@@H]2CCC[C@@H](C)N2c2ccccn2)cc1-c1ncc(F)cn1]

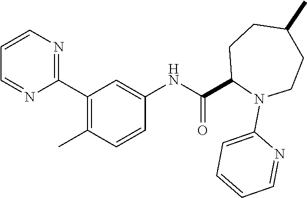

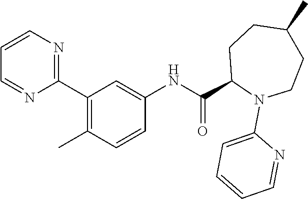 [SMILES: Cc1ccc(NC(=O)[C@H]2CC[C@@H](C)CCN2c2ccccn2)cc1-c1ncccn1]

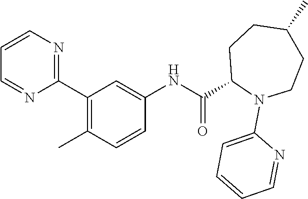 [SMILES: Cc1ccc(NC(=O)[C@@H]2CC[C@H](C)CCN2c2ccccn2)cc1-c1ncccn1]

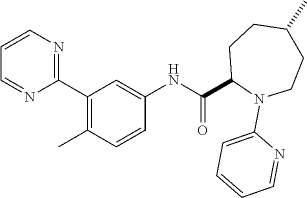

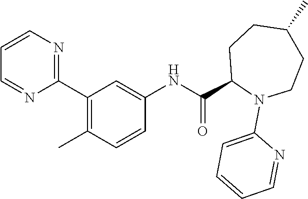 [SMILES: Cc1ccc(NC(=O)[C@H]2CC[C@H](C)CCN2c2ccccn2)cc1-c1ncccn1]

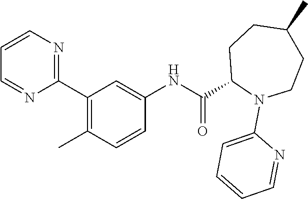 [SMILES: Cc1ccc(NC(=O)[C@@H]2CC[C@@H](C)CCN2c2ccccn2)cc1-c1ncccn1]

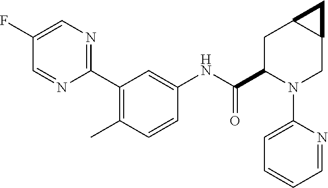

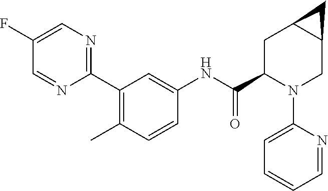 [SMILES: Cc1ccc(NC(=O)[C@H]2C[C@H]3C[C@H]3CN2c2ccccn2)cc1-c1ncc(F)cn1]

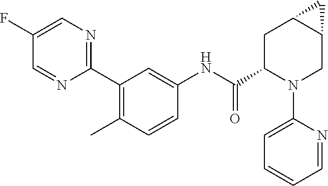 [SMILES: Cc1ccc(NC(=O)[C@@H]2C[C@@H]3C[C@@H]3CN2c2ccccn2)cc1-c1ncc(F)cn1]

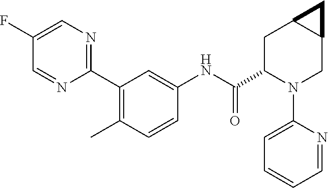

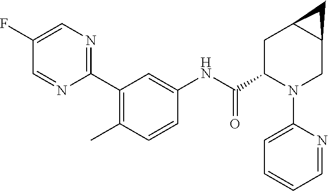 [SMILES: Cc1ccc(NC(=O)[C@@H]2C[C@H]3C[C@H]3CN2c2ccccn2)cc1-c1ncc(F)cn1]

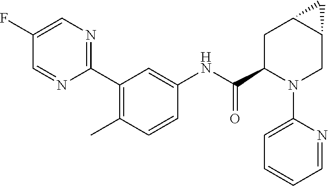 [SMILES: Cc1ccc(NC(=O)[C@H]2C[C@@H]3C[C@@H]3CN2c2ccccn2)cc1-c1ncc(F)cn1]

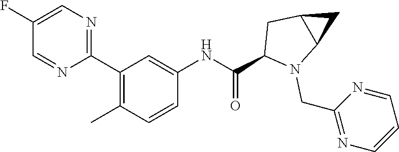 [SMILES: Cc1ccc(NC(=O)[C@H]2C[C@H]3C[C@H]3N2Cc2ncccn2)cc1-c1ncc(F)cn1]

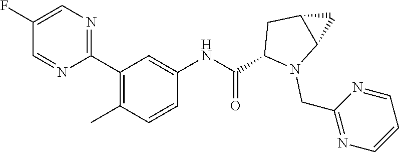 [SMILES: Cc1ccc(NC(=O)[C@@H]2C[C@@H]3C[C@@H]3N2Cc2ncccn2)cc1-c1ncc(F)cn1]

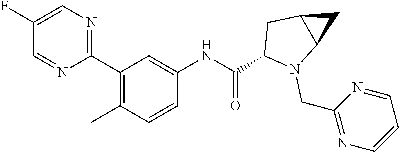 [SMILES: Cc1ccc(NC(=O)[C@@H]2C[C@H]3C[C@H]3N2Cc2ncccn2)cc1-c1ncc(F)cn1]

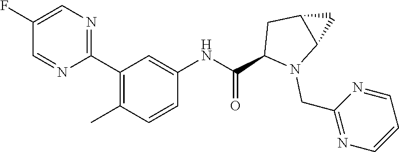 [SMILES: Cc1ccc(NC(=O)[C@H]2C[C@@H]3C[C@@H]3N2Cc2ncccn2)cc1-c1ncc(F)cn1]

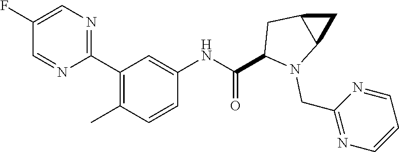 [SMILES: Cc1ccc(NC(=O)[C@H]2C[C@H]3C[C@H]3N2Cc2ncccn2)cc1-c1ncc(F)cn1]

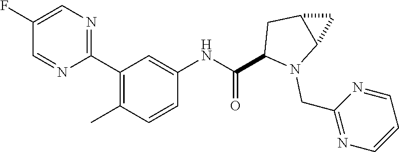 [SMILES: Cc1ccc(NC(=O)[C@H]2C[C@@H]3C[C@@H]3N2Cc2ncccn2)cc1-c1ncc(F)cn1]

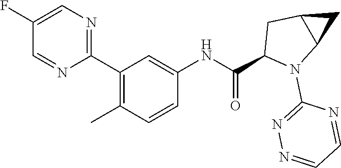 [SMILES: Cc1ccc(NC(=O)[C@H]2C[C@H]3C[C@H]3N2c2nccnn2)cc1-c1ncc(F)cn1]

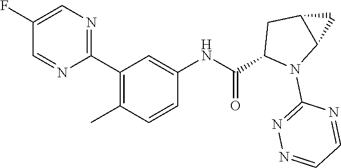 [SMILES: Cc1ccc(NC(=O)[C@@H]2C[C@@H]3C[C@@H]3N2c2nccnn2)cc1-c1ncc(F)cn1]

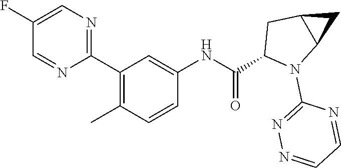 [SMILES: Cc1ccc(NC(=O)[C@@H]2C[C@H]3C[C@H]3N2c2nccnn2)cc1-c1ncc(F)cn1]

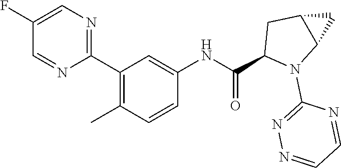 [SMILES: Cc1ccc(NC(=O)[C@H]2C[C@@H]3C[C@@H]3N2c2nccnn2)cc1-c1ncc(F)cn1]

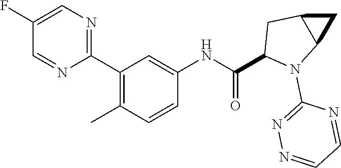 [SMILES: Cc1ccc(NC(=O)[C@H]2C[C@H]3C[C@H]3N2c2nccnn2)cc1-c1ncc(F)cn1]

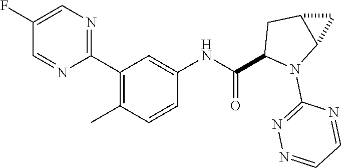 [SMILES: Cc1ccc(NC(=O)[C@H]2C[C@@H]3C[C@@H]3N2c2nccnn2)cc1-c1ncc(F)cn1]

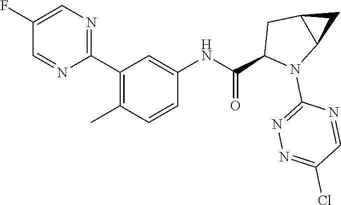 [SMILES: Cc1ccc(NC(=O)[C@H]2C[C@H]3C[C@H]3N2c2ncc(Cl)nn2)cc1-c1ncc(F)cn1]

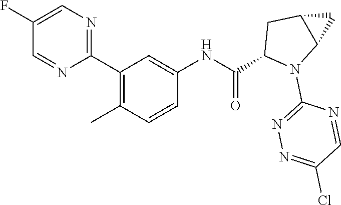 [SMILES: Cc1ccc(NC(=O)[C@@H]2C[C@@H]3C[C@@H]3N2c2ncc(Cl)nn2)cc1-c1ncc(F)cn1]

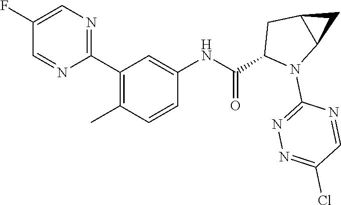 [SMILES: Cc1ccc(NC(=O)[C@@H]2C[C@H]3C[C@H]3N2c2ncc(Cl)nn2)cc1-c1ncc(F)cn1]

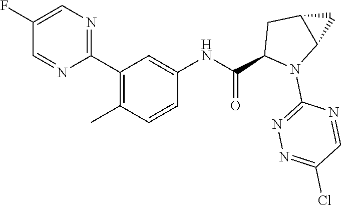 [SMILES: Cc1ccc(NC(=O)[C@H]2C[C@@H]3C[C@@H]3N2c2ncc(Cl)nn2)cc1-c1ncc(F)cn1]

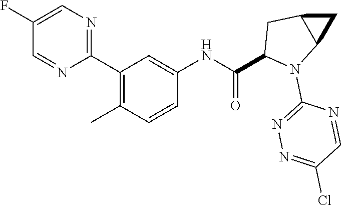 [SMILES: Cc1ccc(NC(=O)[C@H]2C[C@H]3C[C@H]3N2c2ncc(Cl)nn2)cc1-c1ncc(F)cn1]

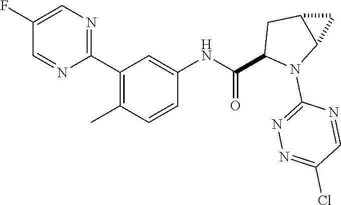 [SMILES: Cc1ccc(NC(=O)[C@H]2C[C@@H]3C[C@@H]3N2c2ncc(Cl)nn2)cc1-c1ncc(F)cn1]

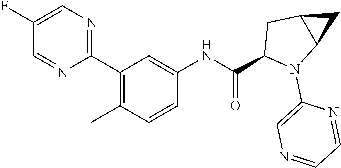 [SMILES: Cc1ccc(NC(=O)[C@H]2C[C@H]3C[C@H]3N2c2cnccn2)cc1-c1ncc(F)cn1]

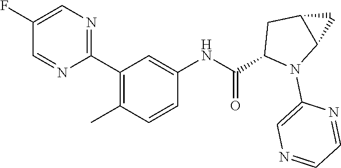 [SMILES: Cc1ccc(NC(=O)[C@@H]2C[C@@H]3C[C@@H]3N2c2cnccn2)cc1-c1ncc(F)cn1]

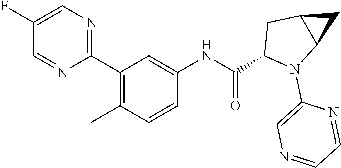 [SMILES: Cc1ccc(NC(=O)[C@@H]2C[C@H]3C[C@H]3N2c2cnccn2)cc1-c1ncc(F)cn1]

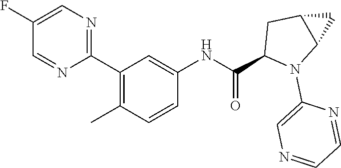 [SMILES: Cc1ccc(NC(=O)[C@H]2C[C@@H]3C[C@@H]3N2c2cnccn2)cc1-c1ncc(F)cn1]

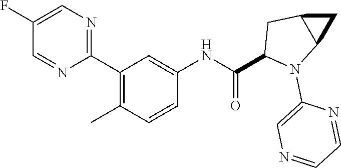 [SMILES: Cc1ccc(NC(=O)[C@H]2C[C@H]3C[C@H]3N2c2cnccn2)cc1-c1ncc(F)cn1]

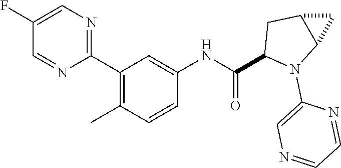 [SMILES: Cc1ccc(NC(=O)[C@H]2C[C@@H]3C[C@@H]3N2c2cnccn2)cc1-c1ncc(F)cn1]

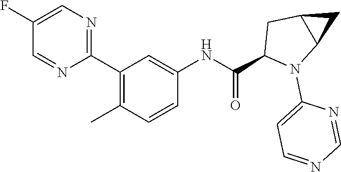 [SMILES: Cc1ccc(NC(=O)[C@H]2C[C@H]3C[C@H]3N2c2ccncn2)cc1-c1ncc(F)cn1]

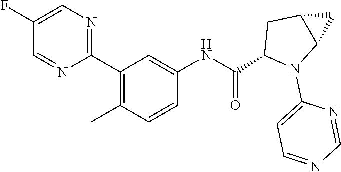 [SMILES: Cc1ccc(NC(=O)[C@@H]2C[C@@H]3C[C@@H]3N2c2ccncn2)cc1-c1ncc(F)cn1]

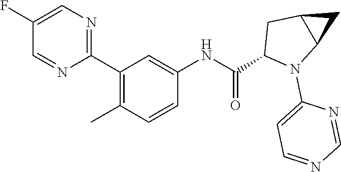 [SMILES: Cc1ccc(NC(=O)[C@@H]2C[C@H]3C[C@H]3N2c2ccncn2)cc1-c1ncc(F)cn1]

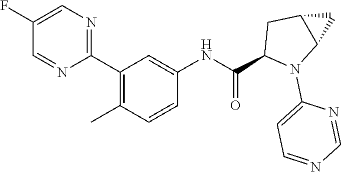 [SMILES: Cc1ccc(NC(=O)[C@H]2C[C@@H]3C[C@@H]3N2c2ccncn2)cc1-c1ncc(F)cn1]

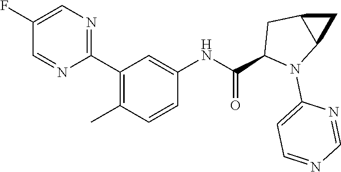 [SMILES: Cc1ccc(NC(=O)[C@H]2C[C@H]3C[C@H]3N2c2ccncn2)cc1-c1ncc(F)cn1]

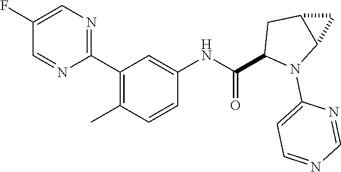 [SMILES: Cc1ccc(NC(=O)[C@H]2C[C@@H]3C[C@@H]3N2c2ccncn2)cc1-c1ncc(F)cn1]

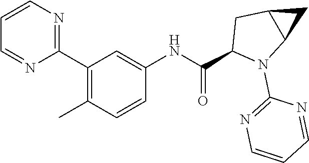 [SMILES: Cc1ccc(NC(=O)[C@H]2C[C@H]3C[C@H]3N2c2ncccn2)cc1-c1ncccn1]

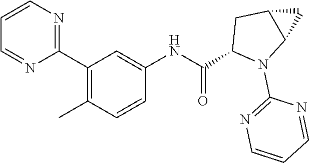 [SMILES: Cc1ccc(NC(=O)[C@@H]2C[C@@H]3C[C@@H]3N2c2ncccn2)cc1-c1ncccn1]

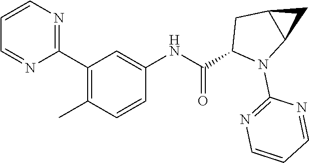 [SMILES: Cc1ccc(NC(=O)[C@@H]2C[C@H]3C[C@H]3N2c2ncccn2)cc1-c1ncccn1]

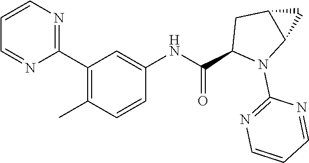 [SMILES: Cc1ccc(NC(=O)[C@H]2C[C@@H]3C[C@@H]3N2c2ncccn2)cc1-c1ncccn1]

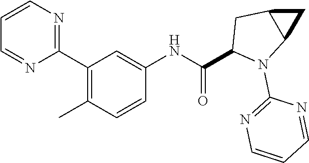 [SMILES: Cc1ccc(NC(=O)[C@H]2C[C@H]3C[C@H]3N2c2ncccn2)cc1-c1ncccn1]

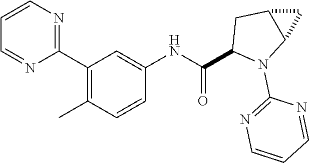 [SMILES: Cc1ccc(NC(=O)[C@H]2C[C@@H]3C[C@@H]3N2c2ncccn2)cc1-c1ncccn1]

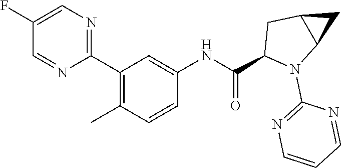 [SMILES: Cc1ccc(NC(=O)[C@H]2C[C@H]3C[C@H]3N2c2ncccn2)cc1-c1ncc(F)cn1]

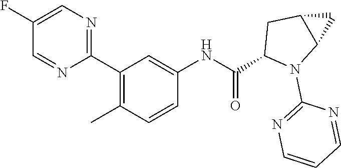 [SMILES: Cc1ccc(NC(=O)[C@@H]2C[C@@H]3C[C@@H]3N2c2ncccn2)cc1-c1ncc(F)cn1]

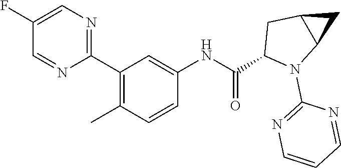 [SMILES: Cc1ccc(NC(=O)[C@@H]2C[C@H]3C[C@H]3N2c2ncccn2)cc1-c1ncc(F)cn1]

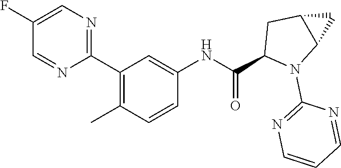 [SMILES: Cc1ccc(NC(=O)[C@H]2C[C@@H]3C[C@@H]3N2c2ncccn2)cc1-c1ncc(F)cn1]

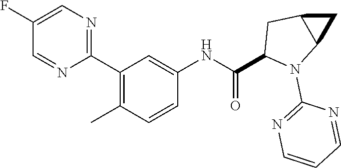 [SMILES: Cc1ccc(NC(=O)[C@H]2C[C@H]3C[C@H]3N2c2ncccn2)cc1-c1ncc(F)cn1]

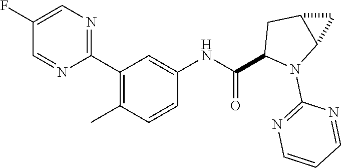 [SMILES: Cc1ccc(NC(=O)[C@H]2C[C@@H]3C[C@@H]3N2c2ncccn2)cc1-c1ncc(F)cn1]

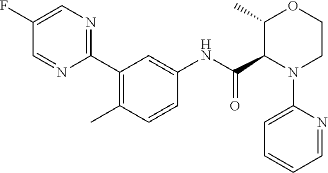 [SMILES: Cc1ccc(NC(=O)[C@H]2[C@H](C)OCCN2c2ccccn2)cc1-c1ncc(F)cn1]

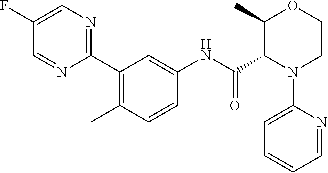 [SMILES: Cc1ccc(NC(=O)[C@@H]2[C@@H](C)OCCN2c2ccccn2)cc1-c1ncc(F)cn1]

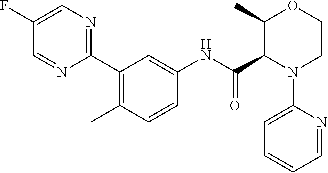 [SMILES: Cc1ccc(NC(=O)[C@H]2[C@@H](C)OCCN2c2ccccn2)cc1-c1ncc(F)cn1]

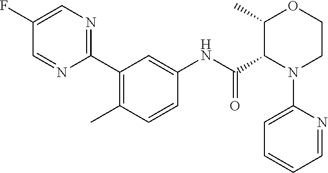 [SMILES: Cc1ccc(NC(=O)[C@@H]2[C@H](C)OCCN2c2ccccn2)cc1-c1ncc(F)cn1]

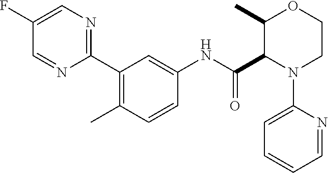

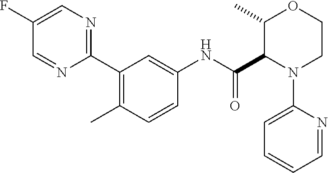

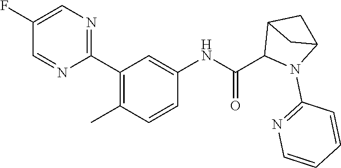 [SMILES: Cc1ccc(NC(=O)C2C3CC(C3)N2c2ccccn2)cc1-c1ncc(F)cn1]

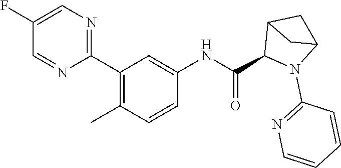 [SMILES: Cc1ccc(NC(=O)[C@H]2C3CC(C3)N2c2ccccn2)cc1-c1ncc(F)cn1]

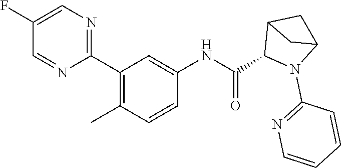 [SMILES: Cc1ccc(NC(=O)[C@@H]2C3CC(C3)N2c2ccccn2)cc1-c1ncc(F)cn1]

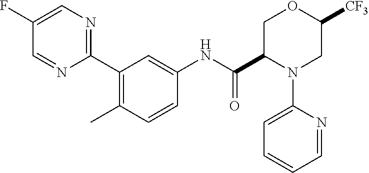

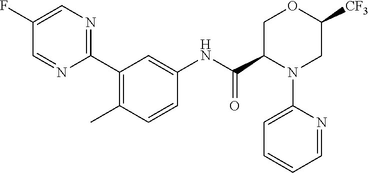 [SMILES: Cc1ccc(NC(=O)[C@H]2CO[C@@H](C(F)(F)F)CN2c2ccccn2)cc1-c1ncc(F)cn1]

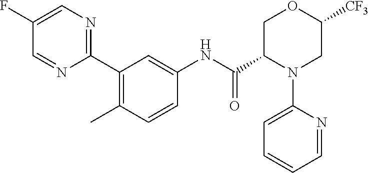 [SMILES: Cc1ccc(NC(=O)[C@@H]2CO[C@H](C(F)(F)F)CN2c2ccccn2)cc1-c1ncc(F)cn1]

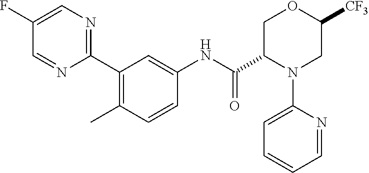

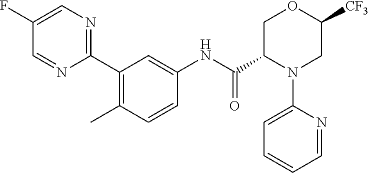 [SMILES: Cc1ccc(NC(=O)[C@@H]2CO[C@@H](C(F)(F)F)CN2c2ccccn2)cc1-c1ncc(F)cn1]

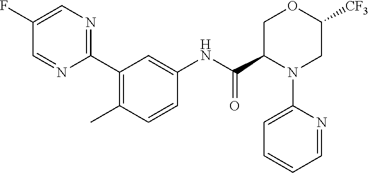 [SMILES: Cc1ccc(NC(=O)[C@H]2CO[C@H](C(F)(F)F)CN2c2ccccn2)cc1-c1ncc(F)cn1]

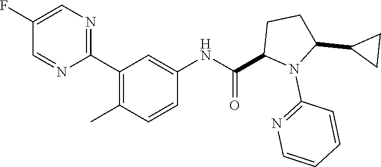 [SMILES: Cc1ccc(NC(=O)[C@H]2CC[C@@H](C3CC3)N2c2ccccn2)cc1-c1ncc(F)cn1]

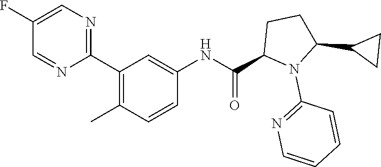 [SMILES: Cc1ccc(NC(=O)[C@H]2CC[C@@H](C3CC3)N2c2ccccn2)cc1-c1ncc(F)cn1]

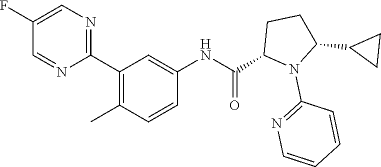 [SMILES: Cc1ccc(NC(=O)[C@@H]2CC[C@H](C3CC3)N2c2ccccn2)cc1-c1ncc(F)cn1]

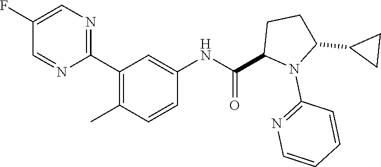 [SMILES: Cc1ccc(NC(=O)[C@H]2CC[C@H](C3CC3)N2c2ccccn2)cc1-c1ncc(F)cn1]

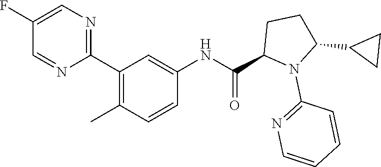 [SMILES: Cc1ccc(NC(=O)[C@H]2CC[C@H](C3CC3)N2c2ccccn2)cc1-c1ncc(F)cn1]

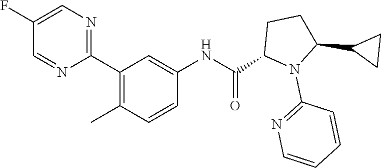 [SMILES: Cc1ccc(NC(=O)[C@@H]2CC[C@@H](C3CC3)N2c2ccccn2)cc1-c1ncc(F)cn1]

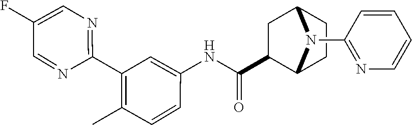

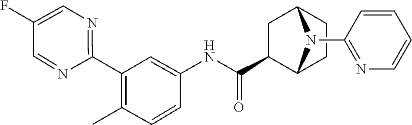 [SMILES: Cc1ccc(NC(=O)[C@H]2C[C@@H]3CC[C@H]2N3c2ccccn2)cc1-c1ncc(F)cn1]

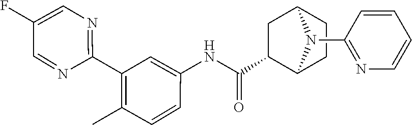 [SMILES: Cc1ccc(NC(=O)[C@@H]2C[C@H]3CC[C@@H]2N3c2ccccn2)cc1-c1ncc(F)cn1]

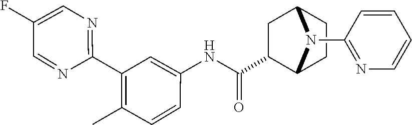

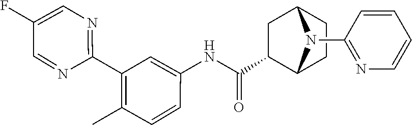 [SMILES: Cc1ccc(NC(=O)[C@@H]2C[C@@H]3CC[C@H]2N3c2ccccn2)cc1-c1ncc(F)cn1]

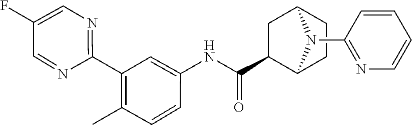 [SMILES: Cc1ccc(NC(=O)[C@H]2C[C@H]3CC[C@@H]2N3c2ccccn2)cc1-c1ncc(F)cn1]

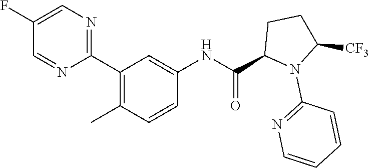 [SMILES: Cc1ccc(NC(=O)[C@H]2CC[C@@H](C(F)(F)F)N2c2ccccn2)cc1-c1ncc(F)cn1]

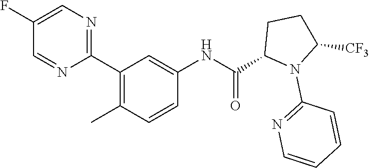 [SMILES: Cc1ccc(NC(=O)[C@@H]2CC[C@H](C(F)(F)F)N2c2ccccn2)cc1-c1ncc(F)cn1]

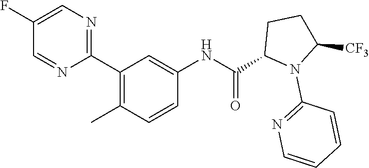 [SMILES: Cc1ccc(NC(=O)[C@@H]2CC[C@@H](C(F)(F)F)N2c2ccccn2)cc1-c1ncc(F)cn1]

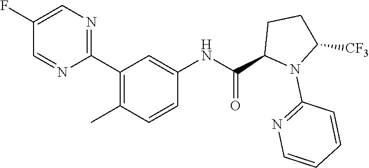 [SMILES: Cc1ccc(NC(=O)[C@H]2CC[C@H](C(F)(F)F)N2c2ccccn2)cc1-c1ncc(F)cn1]

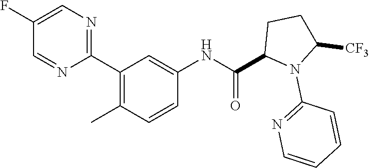 [SMILES: Cc1ccc(NC(=O)[C@H]2CC[C@@H](C(F)(F)F)N2c2ccccn2)cc1-c1ncc(F)cn1]

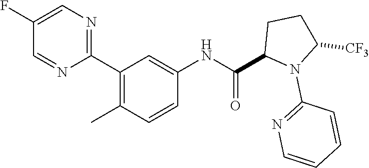 [SMILES: Cc1ccc(NC(=O)[C@H]2CC[C@H](C(F)(F)F)N2c2ccccn2)cc1-c1ncc(F)cn1]

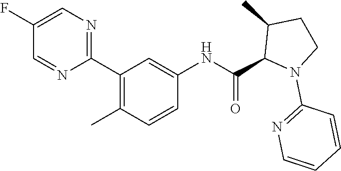 [SMILES: Cc1ccc(NC(=O)[C@H]2[C@@H](C)CCN2c2ccccn2)cc1-c1ncc(F)cn1]

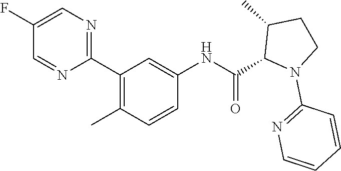 [SMILES: Cc1ccc(NC(=O)[C@@H]2[C@H](C)CCN2c2ccccn2)cc1-c1ncc(F)cn1]

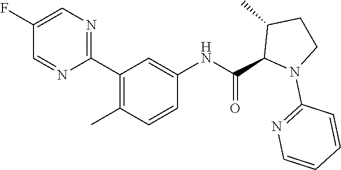 [SMILES: Cc1ccc(NC(=O)[C@H]2[C@H](C)CCN2c2ccccn2)cc1-c1ncc(F)cn1]

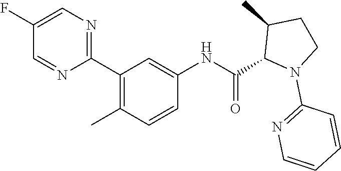 [SMILES: Cc1ccc(NC(=O)[C@@H]2[C@@H](C)CCN2c2ccccn2)cc1-c1ncc(F)cn1]

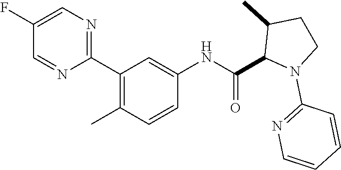 [SMILES: Cc1ccc(NC(=O)[C@H]2[C@@H](C)CCN2c2ccccn2)cc1-c1ncc(F)cn1]

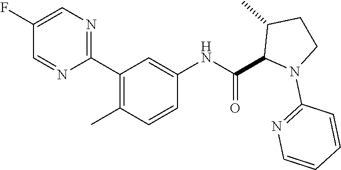 [SMILES: Cc1ccc(NC(=O)[C@H]2[C@H](C)CCN2c2ccccn2)cc1-c1ncc(F)cn1]

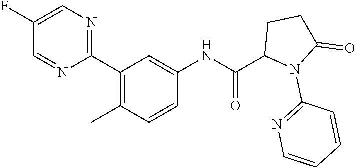 [SMILES: Cc1ccc(NC(=O)C2CCC(=O)N2c2ccccn2)cc1-c1ncc(F)cn1]

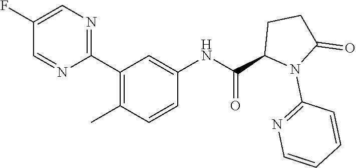 [SMILES: Cc1ccc(NC(=O)[C@H]2CCC(=O)N2c2ccccn2)cc1-c1ncc(F)cn1]

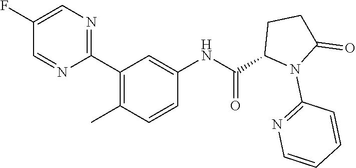 [SMILES: Cc1ccc(NC(=O)[C@@H]2CCC(=O)N2c2ccccn2)cc1-c1ncc(F)cn1]

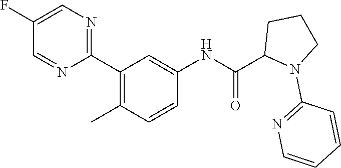 [SMILES: Cc1ccc(NC(=O)C2CCCN2c2ccccn2)cc1-c1ncc(F)cn1]

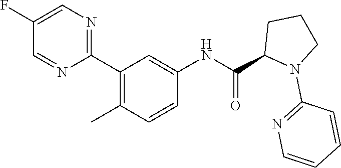 [SMILES: Cc1ccc(NC(=O)[C@H]2CCCN2c2ccccn2)cc1-c1ncc(F)cn1]

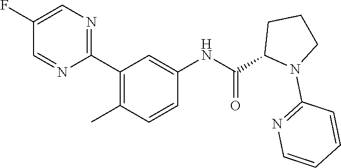 [SMILES: Cc1ccc(NC(=O)[C@@H]2CCCN2c2ccccn2)cc1-c1ncc(F)cn1]

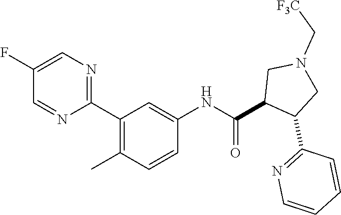

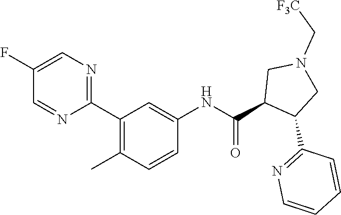 [SMILES: Cc1ccc(NC(=O)[C@H]2CN(CC(F)(F)F)C[C@@H]2c2ccccn2)cc1-c1ncc(F)cn1]

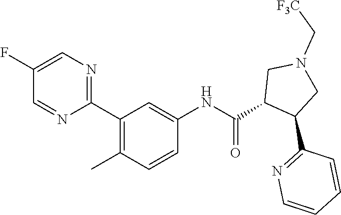 [SMILES: Cc1ccc(NC(=O)[C@@H]2CN(CC(F)(F)F)C[C@H]2c2ccccn2)cc1-c1ncc(F)cn1]

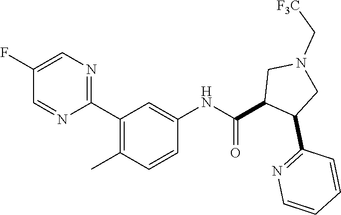

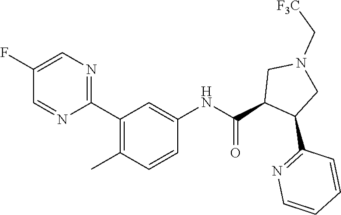 [SMILES: Cc1ccc(NC(=O)[C@H]2CN(CC(F)(F)F)C[C@H]2c2ccccn2)cc1-c1ncc(F)cn1]

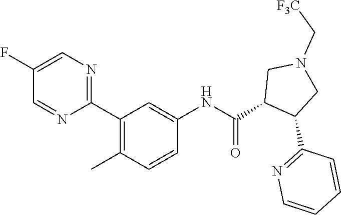 [SMILES: Cc1ccc(NC(=O)[C@@H]2CN(CC(F)(F)F)C[C@@H]2c2ccccn2)cc1-c1ncc(F)cn1]

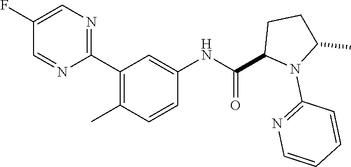 [SMILES: Cc1ccc(NC(=O)[C@H]2CC[C@H](C)N2c2ccccn2)cc1-c1ncc(F)cn1]

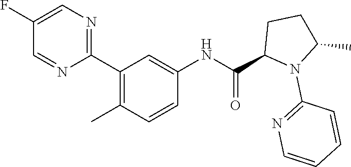 [SMILES: Cc1ccc(NC(=O)[C@H]2CC[C@H](C)N2c2ccccn2)cc1-c1ncc(F)cn1]

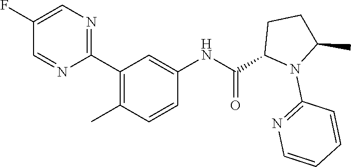 [SMILES: Cc1ccc(NC(=O)[C@@H]2CC[C@@H](C)N2c2ccccn2)cc1-c1ncc(F)cn1]

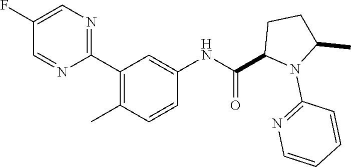 [SMILES: Cc1ccc(NC(=O)[C@H]2CC[C@@H](C)N2c2ccccn2)cc1-c1ncc(F)cn1]

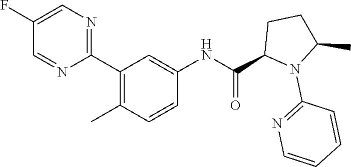 [SMILES: Cc1ccc(NC(=O)[C@H]2CC[C@@H](C)N2c2ccccn2)cc1-c1ncc(F)cn1]

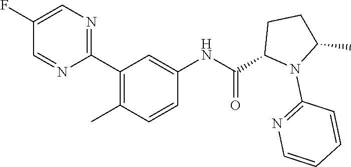 [SMILES: Cc1ccc(NC(=O)[C@@H]2CC[C@H](C)N2c2ccccn2)cc1-c1ncc(F)cn1]

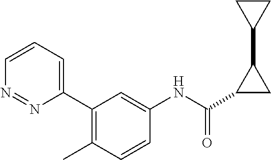 [SMILES: Cc1ccc(NC(=O)[C@H]2C[C@@H]2C2CC2)cc1-c1cccnn1]

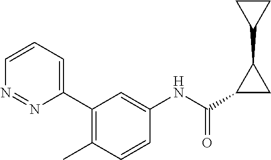 [SMILES: Cc1ccc(NC(=O)[C@H]2C[C@@H]2C2CC2)cc1-c1cccnn1]

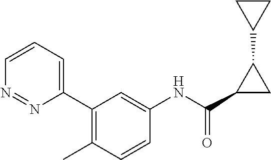 [SMILES: Cc1ccc(NC(=O)[C@@H]2C[C@H]2C2CC2)cc1-c1cccnn1]

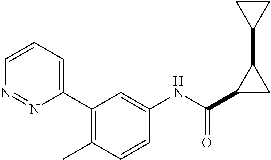 [SMILES: Cc1ccc(NC(=O)[C@@H]2C[C@@H]2C2CC2)cc1-c1cccnn1]

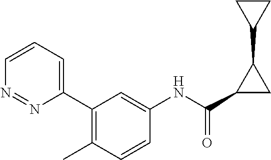 [SMILES: Cc1ccc(NC(=O)[C@@H]2C[C@@H]2C2CC2)cc1-c1cccnn1]

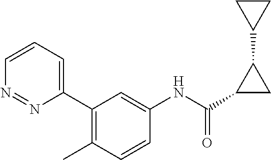 [SMILES: Cc1ccc(NC(=O)[C@H]2C[C@H]2C2CC2)cc1-c1cccnn1]

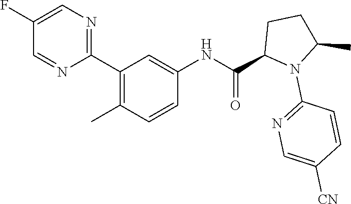 [SMILES: Cc1ccc(NC(=O)[C@H]2CC[C@@H](C)N2c2ccc(C#N)cn2)cc1-c1ncc(F)cn1]

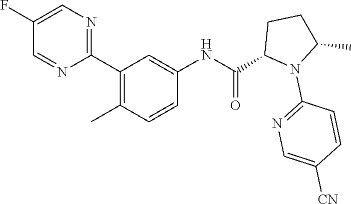 [SMILES: Cc1ccc(NC(=O)[C@@H]2CC[C@H](C)N2c2ccc(C#N)cn2)cc1-c1ncc(F)cn1]

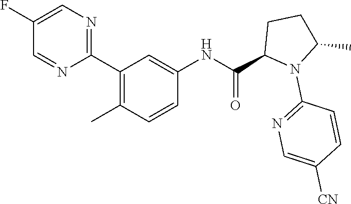 [SMILES: Cc1ccc(NC(=O)[C@H]2CC[C@H](C)N2c2ccc(C#N)cn2)cc1-c1ncc(F)cn1]

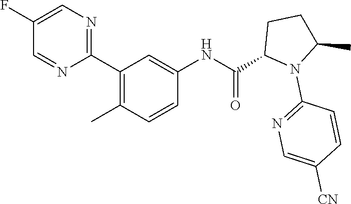 [SMILES: Cc1ccc(NC(=O)[C@@H]2CC[C@@H](C)N2c2ccc(C#N)cn2)cc1-c1ncc(F)cn1]

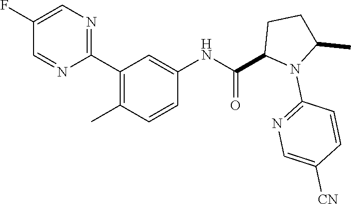 [SMILES: Cc1ccc(NC(=O)[C@H]2CC[C@@H](C)N2c2ccc(C#N)cn2)cc1-c1ncc(F)cn1]

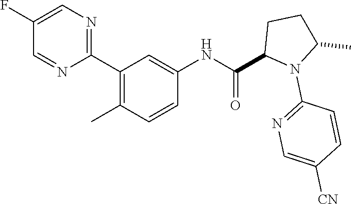 [SMILES: Cc1ccc(NC(=O)[C@H]2CC[C@H](C)N2c2ccc(C#N)cn2)cc1-c1ncc(F)cn1]

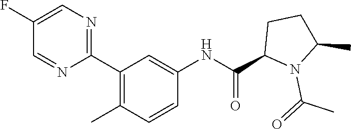 [SMILES: CC(=O)N1[C@H](C)CC[C@@H]1C(=O)Nc1ccc(C)c(-c2ncc(F)cn2)c1]

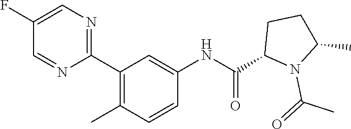 [SMILES: CC(=O)N1[C@@H](C)CC[C@H]1C(=O)Nc1ccc(C)c(-c2ncc(F)cn2)c1]

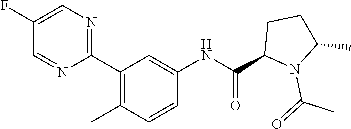 [SMILES: CC(=O)N1[C@@H](C(=O)Nc2ccc(C)c(-c3ncc(F)cn3)c2)CC[C@@H]1C]

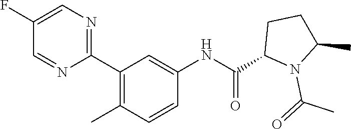 [SMILES: CC(=O)N1[C@H](C)CC[C@H]1C(=O)Nc1ccc(C)c(-c2ncc(F)cn2)c1]

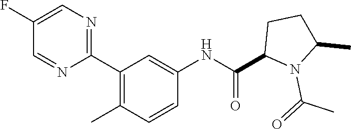 [SMILES: CC(=O)N1[C@H](C)CC[C@@H]1C(=O)Nc1ccc(C)c(-c2ncc(F)cn2)c1]

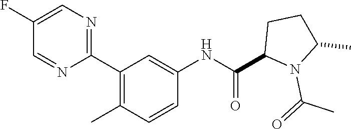 [SMILES: CC(=O)N1[C@@H](C(=O)Nc2ccc(C)c(-c3ncc(F)cn3)c2)CC[C@@H]1C]

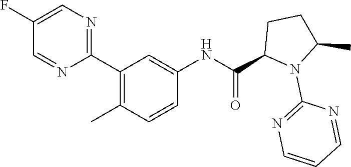 [SMILES: Cc1ccc(NC(=O)[C@H]2CC[C@@H](C)N2c2ncccn2)cc1-c1ncc(F)cn1]

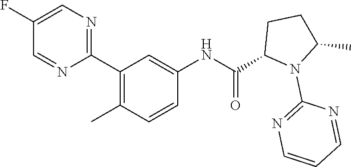 [SMILES: Cc1ccc(NC(=O)[C@@H]2CC[C@H](C)N2c2ncccn2)cc1-c1ncc(F)cn1]

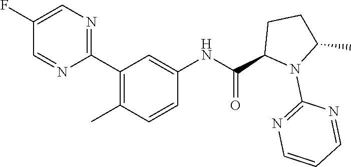 [SMILES: Cc1ccc(NC(=O)[C@H]2CC[C@H](C)N2c2ncccn2)cc1-c1ncc(F)cn1]

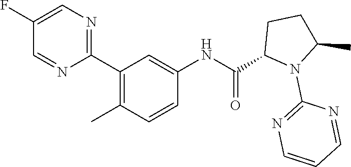 [SMILES: Cc1ccc(NC(=O)[C@@H]2CC[C@@H](C)N2c2ncccn2)cc1-c1ncc(F)cn1]

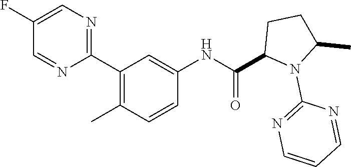 [SMILES: Cc1ccc(NC(=O)[C@H]2CC[C@@H](C)N2c2ncccn2)cc1-c1ncc(F)cn1]

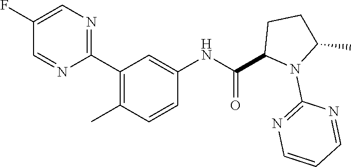 [SMILES: Cc1ccc(NC(=O)[C@H]2CC[C@H](C)N2c2ncccn2)cc1-c1ncc(F)cn1]

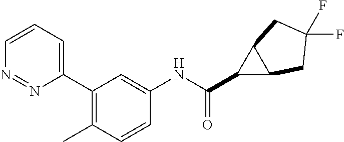

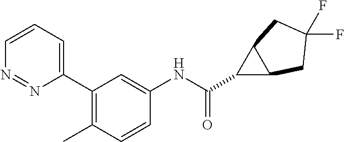

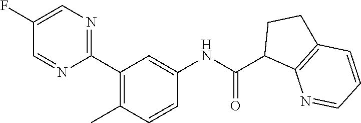 [SMILES: Cc1ccc(NC(=O)C2CCc3cccnc32)cc1-c1ncc(F)cn1]

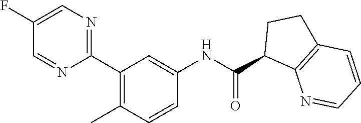 [SMILES: Cc1ccc(NC(=O)[C@H]2CCc3cccnc32)cc1-c1ncc(F)cn1]

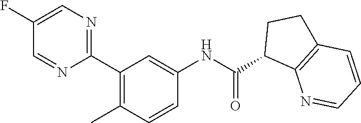 [SMILES: Cc1ccc(NC(=O)[C@@H]2CCc3cccnc32)cc1-c1ncc(F)cn1]

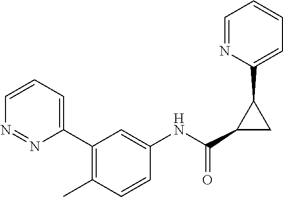 [SMILES: Cc1ccc(NC(=O)[C@@H]2C[C@@H]2c2ccccn2)cc1-c1cccnn1]

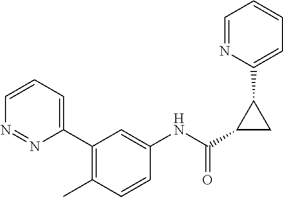 [SMILES: Cc1ccc(NC(=O)[C@H]2C[C@H]2c2ccccn2)cc1-c1cccnn1]

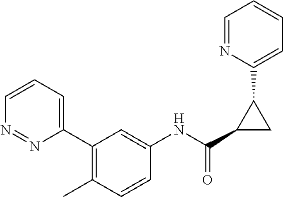 [SMILES: Cc1ccc(NC(=O)[C@@H]2C[C@H]2c2ccccn2)cc1-c1cccnn1]

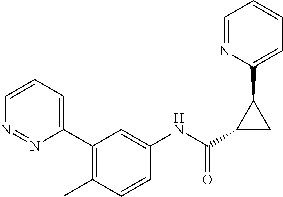 [SMILES: Cc1ccc(NC(=O)[C@H]2C[C@@H]2c2ccccn2)cc1-c1cccnn1]

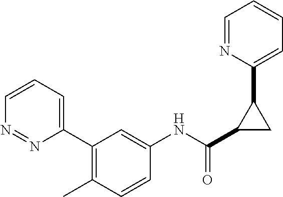 [SMILES: Cc1ccc(NC(=O)[C@@H]2C[C@@H]2c2ccccn2)cc1-c1cccnn1]

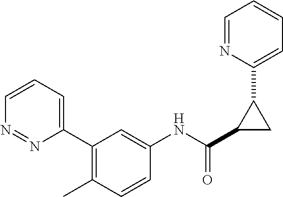 [SMILES: Cc1ccc(NC(=O)[C@@H]2C[C@H]2c2ccccn2)cc1-c1cccnn1]

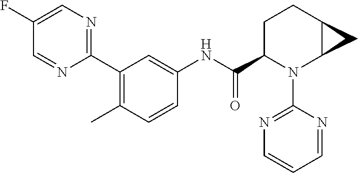 [SMILES: Cc1ccc(NC(=O)[C@H]2CC[C@H]3C[C@H]3N2c2ncccn2)cc1-c1ncc(F)cn1]

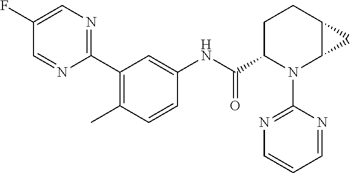 [SMILES: Cc1ccc(NC(=O)[C@@H]2CC[C@@H]3C[C@@H]3N2c2ncccn2)cc1-c1ncc(F)cn1]

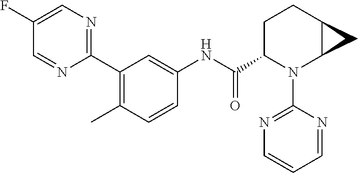 [SMILES: Cc1ccc(NC(=O)[C@@H]2CC[C@H]3C[C@H]3N2c2ncccn2)cc1-c1ncc(F)cn1]

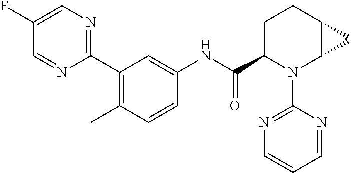 [SMILES: Cc1ccc(NC(=O)[C@H]2CC[C@@H]3C[C@@H]3N2c2ncccn2)cc1-c1ncc(F)cn1]

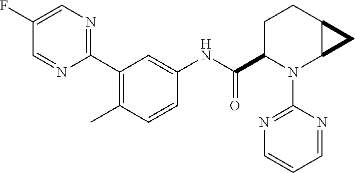

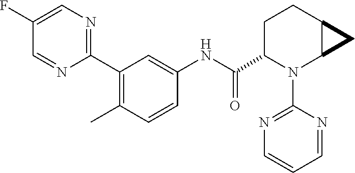

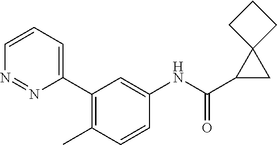 [SMILES: Cc1ccc(NC(=O)C2CC23CCC3)cc1-c1cccnn1]

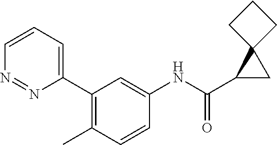 [SMILES: Cc1ccc(NC(=O)[C@@H]2CC23CCC3)cc1-c1cccnn1]

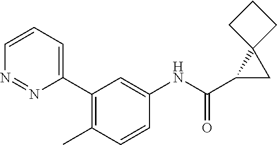 [SMILES: Cc1ccc(NC(=O)[C@H]2CC23CCC3)cc1-c1cccnn1]

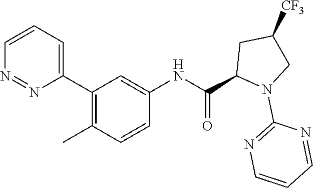 [SMILES: Cc1ccc(NC(=O)[C@H]2C[C@@H](C(F)(F)F)CN2c2ncccn2)cc1-c1cccnn1]

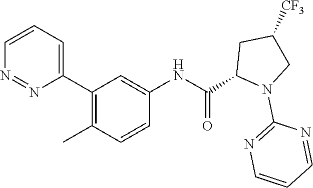 [SMILES: Cc1ccc(NC(=O)[C@@H]2C[C@H](C(F)(F)F)CN2c2ncccn2)cc1-c1cccnn1]

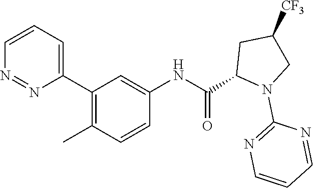 [SMILES: Cc1ccc(NC(=O)[C@@H]2C[C@@H](C(F)(F)F)CN2c2ncccn2)cc1-c1cccnn1]

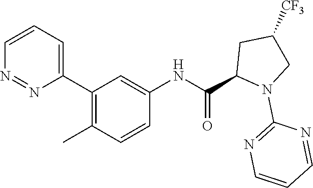 [SMILES: Cc1ccc(NC(=O)[C@H]2C[C@H](C(F)(F)F)CN2c2ncccn2)cc1-c1cccnn1]

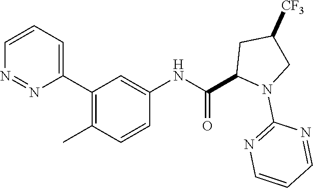 [SMILES: Cc1ccc(NC(=O)[C@H]2C[C@@H](C(F)(F)F)CN2c2ncccn2)cc1-c1cccnn1]

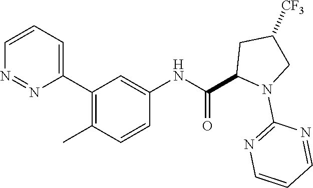 [SMILES: Cc1ccc(NC(=O)[C@H]2C[C@H](C(F)(F)F)CN2c2ncccn2)cc1-c1cccnn1]

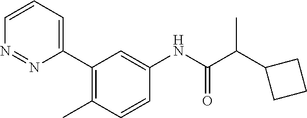 [SMILES: Cc1ccc(NC(=O)C(C)C2CCC2)cc1-c1cccnn1]

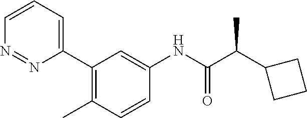 [SMILES: Cc1ccc(NC(=O)[C@@H](C)C2CCC2)cc1-c1cccnn1]

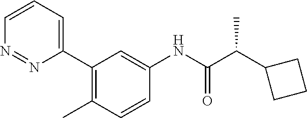 [SMILES: Cc1ccc(NC(=O)[C@H](C)C2CCC2)cc1-c1cccnn1]

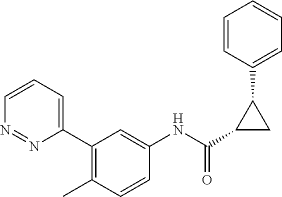 [SMILES: Cc1ccc(NC(=O)[C@H]2C[C@H]2c2ccccc2)cc1-c1cccnn1]

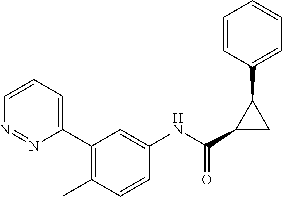 [SMILES: Cc1ccc(NC(=O)[C@@H]2C[C@@H]2c2ccccc2)cc1-c1cccnn1]

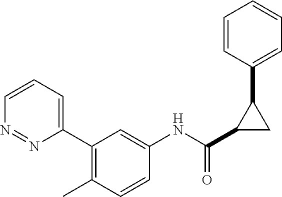 [SMILES: Cc1ccc(NC(=O)[C@@H]2C[C@@H]2c2ccccc2)cc1-c1cccnn1]

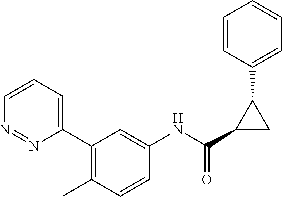 [SMILES: Cc1ccc(NC(=O)[C@@H]2C[C@H]2c2ccccc2)cc1-c1cccnn1]

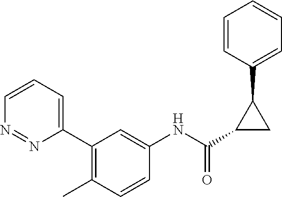 [SMILES: Cc1ccc(NC(=O)[C@H]2C[C@@H]2c2ccccc2)cc1-c1cccnn1]

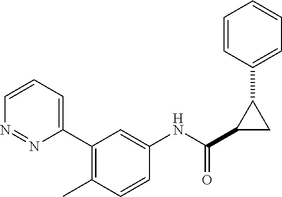 [SMILES: Cc1ccc(NC(=O)[C@@H]2C[C@H]2c2ccccc2)cc1-c1cccnn1]

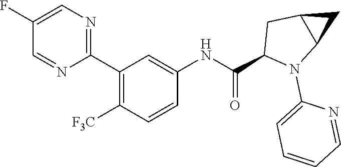 [SMILES: O=C(Nc1ccc(C(F)(F)F)c(-c2ncc(F)cn2)c1)[C@H]1C[C@H]2C[C@H]2N1c1ccccn1]

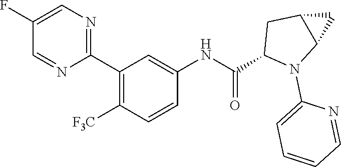 [SMILES: O=C(Nc1ccc(C(F)(F)F)c(-c2ncc(F)cn2)c1)[C@@H]1C[C@@H]2C[C@@H]2N1c1ccccn1]

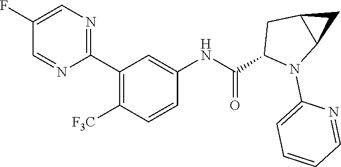 [SMILES: O=C(Nc1ccc(C(F)(F)F)c(-c2ncc(F)cn2)c1)[C@@H]1C[C@H]2C[C@H]2N1c1ccccn1]

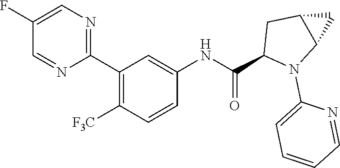 [SMILES: O=C(Nc1ccc(C(F)(F)F)c(-c2ncc(F)cn2)c1)[C@H]1C[C@@H]2C[C@@H]2N1c1ccccn1]

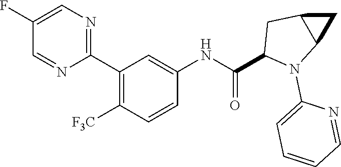 [SMILES: O=C(Nc1ccc(C(F)(F)F)c(-c2ncc(F)cn2)c1)[C@H]1C[C@H]2C[C@H]2N1c1ccccn1]

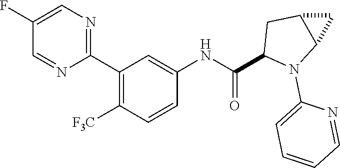

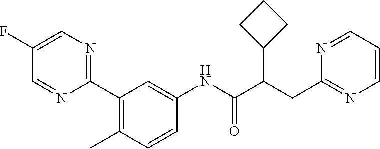 [SMILES: Cc1ccc(NC(=O)C(Cc2ncccn2)C2CCC2)cc1-c1ncc(F)cn1]

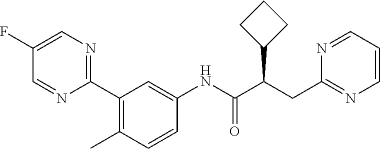 [SMILES: Cc1ccc(NC(=O)[C@H](Cc2ncccn2)C2CCC2)cc1-c1ncc(F)cn1]

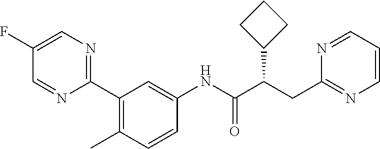 [SMILES: Cc1ccc(NC(=O)[C@@H](Cc2ncccn2)C2CCC2)cc1-c1ncc(F)cn1]

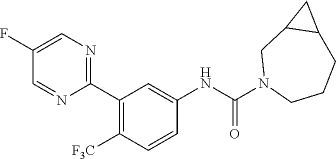 [SMILES: O=C(Nc1ccc(C(F)(F)F)c(-c2ncc(F)cn2)c1)N1CCCC2CC2C1]

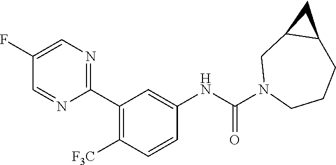 [SMILES: O=C(Nc1ccc(C(F)(F)F)c(-c2ncc(F)cn2)c1)N1CCC[C@@H]2C[C@@H]2C1]

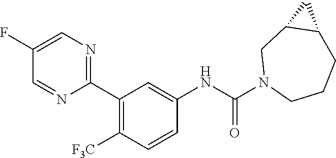 [SMILES: O=C(Nc1ccc(C(F)(F)F)c(-c2ncc(F)cn2)c1)N1CCC[C@H]2C[C@H]2C1]

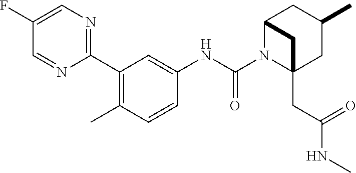

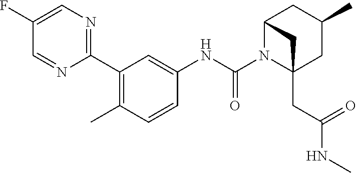 [SMILES: CNC(=O)C[C@]12C[C@H](C)C[C@H](C1)N2C(=O)Nc1ccc(C)c(-c2ncc(F)cn2)c1]

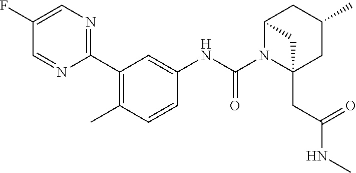 [SMILES: CNC(=O)C[C@@]12C[C@@H](C)C[C@@H](C1)N2C(=O)Nc1ccc(C)c(-c2ncc(F)cn2)c1]

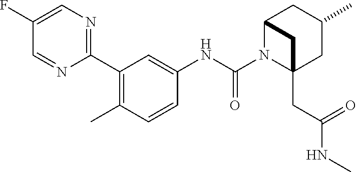

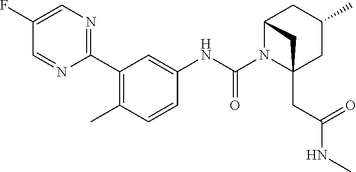 [SMILES: CNC(=O)C[C@]12C[C@@H](C)C[C@H](C1)N2C(=O)Nc1ccc(C)c(-c2ncc(F)cn2)c1]

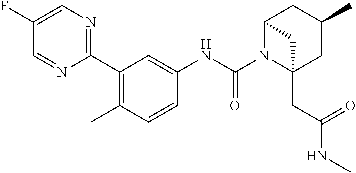 [SMILES: CNC(=O)C[C@@]12C[C@H](C)C[C@@H](C1)N2C(=O)Nc1ccc(C)c(-c2ncc(F)cn2)c1]

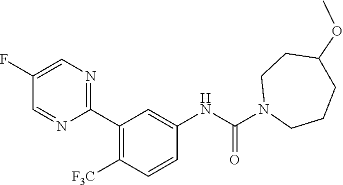 [SMILES: COC1CCCN(C(=O)Nc2ccc(C(F)(F)F)c(-c3ncc(F)cn3)c2)CC1]

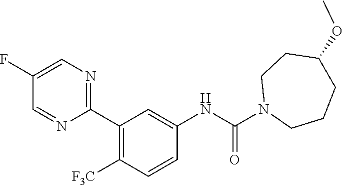 [SMILES: CO[C@@H]1CCCN(C(=O)Nc2ccc(C(F)(F)F)c(-c3ncc(F)cn3)c2)CC1]

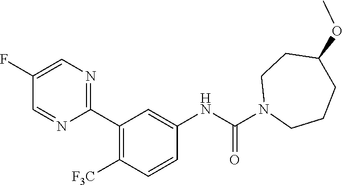 [SMILES: CO[C@H]1CCCN(C(=O)Nc2ccc(C(F)(F)F)c(-c3ncc(F)cn3)c2)CC1]

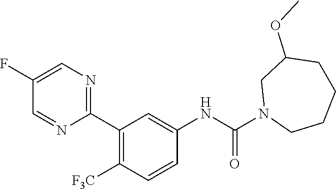 [SMILES: COC1CCCCN(C(=O)Nc2ccc(C(F)(F)F)c(-c3ncc(F)cn3)c2)C1]

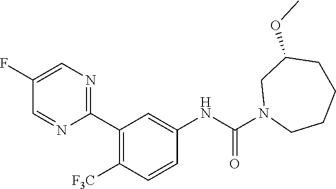 [SMILES: CO[C@@H]1CCCCN(C(=O)Nc2ccc(C(F)(F)F)c(-c3ncc(F)cn3)c2)C1]

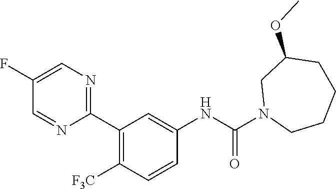 [SMILES: CO[C@H]1CCCCN(C(=O)Nc2ccc(C(F)(F)F)c(-c3ncc(F)cn3)c2)C1]

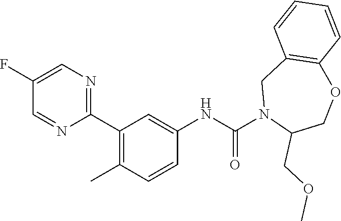 [SMILES: COCC1COc2ccccc2CN1C(=O)Nc1ccc(C)c(-c2ncc(F)cn2)c1]

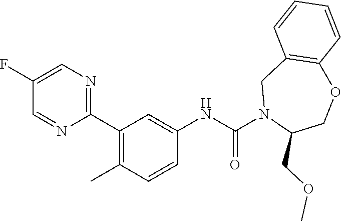 [SMILES: COC[C@@H]1COc2ccccc2CN1C(=O)Nc1ccc(C)c(-c2ncc(F)cn2)c1]

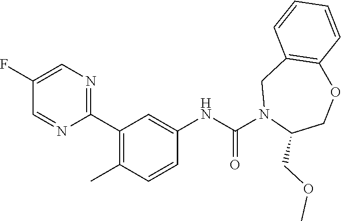 [SMILES: COC[C@H]1COc2ccccc2CN1C(=O)Nc1ccc(C)c(-c2ncc(F)cn2)c1]

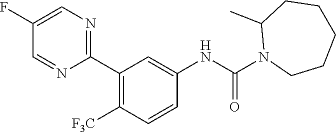 [SMILES: CC1CCCCCN1C(=O)Nc1ccc(C(F)(F)F)c(-c2ncc(F)cn2)c1]

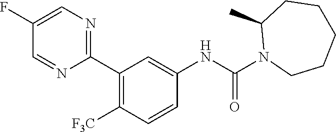 [SMILES: C[C@H]1CCCCCN1C(=O)Nc1ccc(C(F)(F)F)c(-c2ncc(F)cn2)c1]

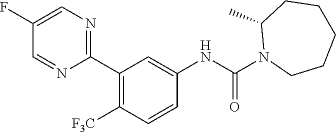 [SMILES: C[C@@H]1CCCCCN1C(=O)Nc1ccc(C(F)(F)F)c(-c2ncc(F)cn2)c1]

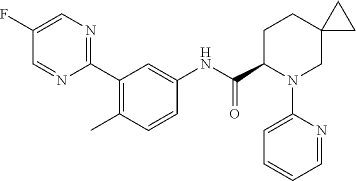 [SMILES: Cc1ccc(NC(=O)[C@H]2CCC3(CC3)CN2c2ccccn2)cc1-c1ncc(F)cn1]

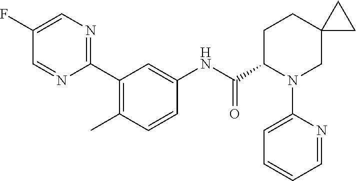 [SMILES: Cc1ccc(NC(=O)[C@@H]2CCC3(CC3)CN2c2ccccn2)cc1-c1ncc(F)cn1]

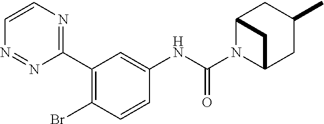

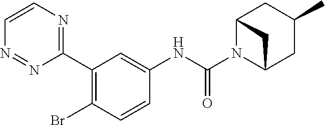 [SMILES: C[C@@H]1C[C@@H]2C[C@H](C1)N2C(=O)Nc1ccc(Br)c(-c2nccnn2)c1]

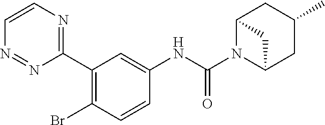 [SMILES: C[C@@H]1C[C@@H]2C[C@H](C1)N2C(=O)Nc1ccc(Br)c(-c2nccnn2)c1]

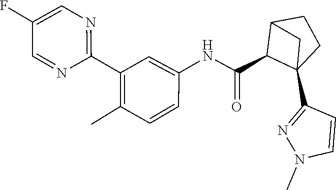 [SMILES: Cc1ccc(NC(=O)[C@H]2C3CC[C@@]2(c2ccn(C)n2)C3)cc1-c1ncc(F)cn1]

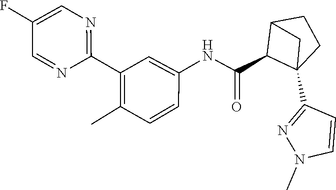 [SMILES: Cc1ccc(NC(=O)[C@H]2C3CC[C@]2(c2ccn(C)n2)C3)cc1-c1ncc(F)cn1]

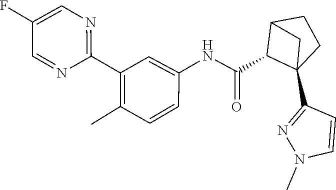 [SMILES: Cc1ccc(NC(=O)[C@@H]2C3CC[C@@]2(c2ccn(C)n2)C3)cc1-c1ncc(F)cn1]

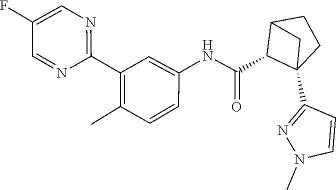 [SMILES: Cc1ccc(NC(=O)[C@@H]2C3CC[C@]2(c2ccn(C)n2)C3)cc1-c1ncc(F)cn1]

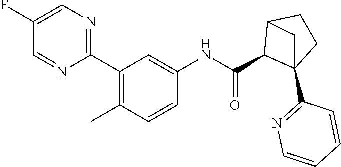 [SMILES: Cc1ccc(NC(=O)[C@H]2C3CC[C@@]2(c2ccccn2)C3)cc1-c1ncc(F)cn1]

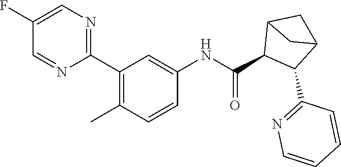 [SMILES: Cc1ccc(NC(=O)[C@H]2C3CC(C3)[C@@H]2c2ccccn2)cc1-c1ncc(F)cn1]

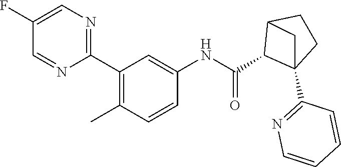 [SMILES: Cc1ccc(NC(=O)[C@@H]2C3CC[C@]2(c2ccccn2)C3)cc1-c1ncc(F)cn1]

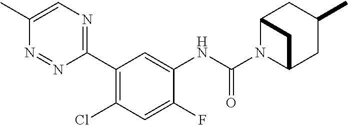

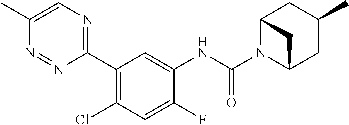

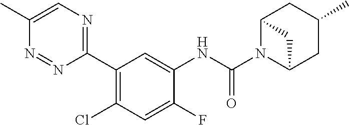

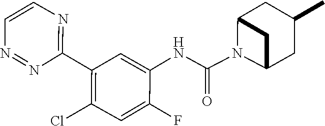 [SMILES: C[C@@H]1C[C@@H]2C[C@H](C1)N2C(=O)Nc1cc(-c2nccnn2)c(Cl)cc1F]

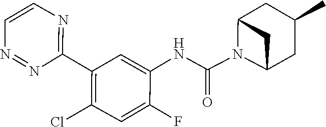 [SMILES: C[C@@H]1C[C@@H]2C[C@H](C1)N2C(=O)Nc1cc(-c2nccnn2)c(Cl)cc1F]

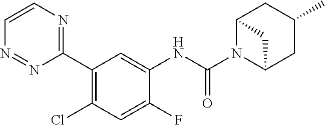 [SMILES: C[C@@H]1C[C@@H]2C[C@H](C1)N2C(=O)Nc1cc(-c2nccnn2)c(Cl)cc1F]

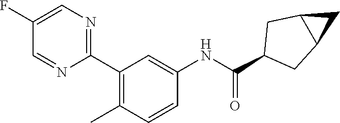 [SMILES: Cc1ccc(NC(=O)[C@@H]2C[C@@H]3C[C@@H]3C2)cc1-c1ncc(F)cn1]

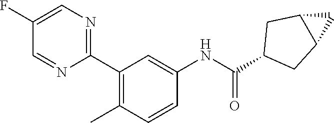 [SMILES: Cc1ccc(NC(=O)[C@@H]2C[C@@H]3C[C@@H]3C2)cc1-c1ncc(F)cn1]

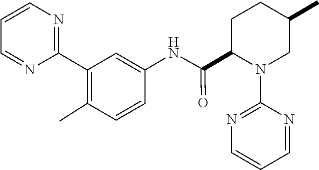 [SMILES: Cc1ccc(NC(=O)[C@H]2CC[C@@H](C)CN2c2ncccn2)cc1-c1ncccn1]

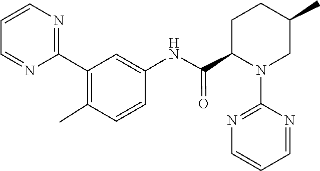 [SMILES: Cc1ccc(NC(=O)[C@H]2CC[C@@H](C)CN2c2ncccn2)cc1-c1ncccn1]

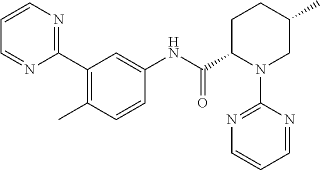 [SMILES: Cc1ccc(NC(=O)[C@@H]2CC[C@H](C)CN2c2ncccn2)cc1-c1ncccn1]

### 3. Methods

“Treatment” or “treating” is an approach for obtaining beneficial or desired results including clinical results. Beneficial or desired clinical results may include one or more of the following: a) inhibiting the disease or condition (e.g., decreasing one or more symptoms resulting from the disease or condition, and/or diminishing the extent of the disease or condition); b) slowing or arresting the development of one or more clinical symptoms associated with the disease or condition (e.g., stabilizing the disease or condition, preventing or delaying the worsening or progression of the disease or condition, and/or preventing or delaying the spread (e.g., metastasis) of the disease or condition); and/or c) relieving the disease, that is, causing the regression of clinical symptoms (e.g., ameliorating the disease state, providing partial or total remission of the disease or condition, enhancing effect of another medication, delaying the progression of the disease, increasing the quality of life, and/or prolonging survival.

“Prevention” or “preventing” means any treatment of a disease or condition that causes the clinical symptoms of the disease or condition not to develop. Compounds may, in certain embodiments, be administered to a subject (including a human) who is at risk or has a family history of the disease or condition.

“Subject” refers to an animal, such as a mammal (including a human), that has been or will be the object of treatment, observation or experiment. The methods described herein may be useful in human therapy, and/or veterinary applications. In certain embodiments, the subject is a mammal. In certain embodiments, the subject is a human.

The term “therapeutically effective amount” or “effective amount” of a compound described herein or a pharmaceutically acceptable salt, isotopically enriched analog, stereoisomer, mixture of stereoisomers, or prodrug thereof means an amount sufficient to effect treatment when administered to a subject, to provide a therapeutic benefit such as amelioration of symptoms or slowing of disease progression. For example, a therapeutically effective amount may be an amount sufficient to decrease a symptom of a disease or condition of as described herein. The therapeutically effective amount may vary depending on the subject, and disease or condition being treated, the weight and age of the subject, the severity of the disease or condition, and the manner of administering, which can readily be determined by one of ordinary skill in the art.

The methods described herein may be applied to cell populations in vivo or ex vivo. “In vivo” means within a living individual, as within an animal or human. In this context, the methods described herein may be used therapeutically in an individual. “Ex vivo” means outside of a living individual. Examples of ex vivo cell populations include in vitro cell cultures and biological samples including fluid or tissue samples obtained from individuals. Such samples may be obtained by methods well known in the art. Exemplary biological fluid samples include blood, cerebrospinal fluid, urine, and saliva. In this context, the compounds and compositions described herein may be used for a variety of purposes, including therapeutic and experimental purposes. For example, the compounds and compositions described herein may be used ex vivo to determine the optimal schedule and/or dosing of administration of a compound of the present disclosure for a given indication, cell type, individual, and other parameters. Information gleaned from such use may be used for experimental purposes or in the clinic to set protocols for in vivo treatment. Other ex vivo uses for which the compounds and compositions described herein may be suited are described below or will become apparent to those skilled in the art.

The compounds may be further characterized to examine the safety or tolerance dosage in human or non-human subjects. Such properties may be examined using commonly known methods to those skilled in the art.

In certain embodiments, provided are compounds, or a pharmaceutically acceptable salt, isotopically enriched analog, stereoisomer, mixture of stereoisomers, or prodrug thereof, that inhibit the activity of Sterile Alpha and TIR Motif containing 1 (SARM1) protein. In certain embodiments, the compounds provided herein, or a pharmaceutically acceptable salt, isotopically enriched analog, stereoisomer, mixture of stereoisomers, or prodrug thereof, inhibits SARM1.

In certain methods, uses and compositions provided herein, the compound is a compound of Formula I:

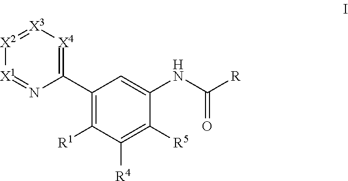

or a pharmaceutically acceptable salt, isotopically enriched analog, stereoisomer, tautomer, mixture of stereoisomers thereof, wherein:
each of X¹, X², X³, and X⁴ are independently N or CR⁶; provided no more than two of X¹, X², X³, and X⁴ are N;
R is C₁₋₆ alkyl, —NR²R³, —O—R⁷, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹;
R¹ is hydrogen, halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R² is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R³ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
or R² and R³ together form a heterocyclyl, which may further be independently optionally substituted with one to five Z¹;
R⁴ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R⁵ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁶ is independently hydrogen, halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R⁷ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁸ is independently halo, cyano, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₃₋₁₀ cycloalkyl, heterocyclyl, aryl, or heteroaryl is independently optionally substituted with one to five Z¹;
each R¹¹ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z¹ is independently halo, cyano, —NO₂, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, —N(R¹²)₂, —OR¹², —SR¹², —C(O)R¹², —C(O)OR¹², —S(O)R¹², —S(O)₂R¹², —C(O)N(R¹²)₂, —NR¹²C(O)R¹², —NR¹²S(O)R¹², —NR¹²S(O)₂R¹², —S(O)N(R¹²)₂, —S(O)₂N(R¹²)₂, —NR¹²C(O)N(R¹²)₂, —NR¹²S(O)N(R¹²)₂, —NR¹²S(O)₂N(R¹²)₂, —OC(O)N(R¹²)₂, or —NR¹²C(O)OR¹²; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with one to five Z^{1a};
each R¹² is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1a} is independently halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —SR¹³, —C(O)R¹³, —C(O)OR¹³, —S(O)R¹³, —S(O)₂R¹³, —C(O)N(R¹³)₂, —NR¹³C(O)R¹³, —NR¹³S(O)R¹³, —NR¹³S(O)₂R¹³, —S(O)N(R¹³)₂, —S(O)₂N(R¹³)₂, —NR¹³C(O)N(R¹³)₂, —NR¹³S(O)N(R¹³)₂, —NR¹³S(O)₂N(R¹³)₂, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1b} is independently halo, cyano, —OH, —SH, —NH₂, —NO₂, C₁₋₆ alkyl, C₁₋₆ haloalkyl, C₂₋₆ alkenyl, C₂₋₆ alkynyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five halo, cyano, —OH, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl.

In certain methods, uses and compositions provided herein, the compound is a compound of Formula II, or a pharmaceutically acceptable salt, isotopically enriched analog, stereoisomer, tautomer, mixture of stereoisomers thereof, wherein:

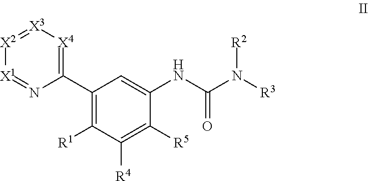

or a pharmaceutically acceptable salt, isotopically enriched analog, stereoisomer, tautomer, mixture of stereoisomers thereof, wherein:
each of X¹, X², X³, and X⁴ are independently N or CR⁶; provided no more than two of X¹, X², X³, and X⁴ are N;
R¹ is halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R² is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R³ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
or R² and R³ together form a heterocyclyl, which may further be independently optionally substituted with one to five Z¹;
R⁴ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R⁵ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁶ is independently hydrogen, halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
each R¹¹ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z¹ is independently halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹²)₂, —OR¹², —SR¹², —C(O)R¹², —C(O)OR¹², —S(O)R¹², —S(O)₂R¹², —C(O)N(R¹²)₂, —NR¹²C(O)R¹², —NR¹²S(O)R¹², —NR¹²S(O)₂R¹², —S(O)N(R¹²)₂, —S(O)₂N(R¹²)₂, —NR¹²C(O)N(R¹²)₂, —NR¹²S(O)N(R¹²)₂, —NR¹²S(O)₂N(R¹²)₂, —OC(O)N(R¹²)₂, or —NR¹²C(O)OR¹²; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each R¹² is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1a} is independently halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —SR¹³, —C(O)R¹³, —C(O)OR¹³, —S(O)R¹³, —S(O)₂R¹³, —C(O)N(R¹³)₂, —NR¹³C(O)R¹³, —NR¹³S(O)R¹³, —NR¹³S(O)₂R¹³, —S(O)N(R¹³)₂, —S(O)₂N(R¹³)₂, —NR¹³C(O)N(R¹³)₂, —NR¹³S(O)N(R¹³)₂, —NR¹³S(O)₂N(R¹³)₂, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1b} is independently halo, cyano, —OH, —SH, —NH₂, —NO₂, C₁₋₆ alkyl, C₁₋₆ haloalkyl, C₂₋₆ alkenyl, C₂₋₆ alkynyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five halo, cyano, —OH, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl;

In certain embodiments, the compound is not N-[3-(2-amino-4-pyrimidinyl)-4-fluorophenyl]-1-pyrrolidinecarboxamide.

In certain embodiments, the compound is not N-[3-(3-amino-1,2,4-triazin-5-yl)-4-fluorophenyl]-1-pyrrolidinecarboxamide.

In certain embodiments, the compound is not 2-[4-methyl-6-(4-morpholinyl)-1,3,5-triazin-2-yl]-4-[(4-morpholinylcarbonyl)amino]benzenesulfonic acid.

In certain embodiments, the compound is not N-[4-fluoro-3-[2-[3-(hydroxymethyl)phenyl]-6-(4-morpholinyl)-4-pyrimidinyl]phenyl]-1-piperazinecarboxamide.

In certain embodiments, the compound is not or 2,3-dihydro-5-methoxy-N-[4-methyl-3-(2-pyridinyl)phenyl]-6-(trifluoromethyl)-1H-indole-1-carboxamide.

In certain embodiments, when X² is N, R¹ is methyl, R⁴ is hydrogen, and R⁵ is hydrogen, then neither of X¹ and X³ are C-morpholino.

In certain embodiments, provided is a method of inhibiting SARM1 activity comprising contacting a cell with an effective amount of a compound disclosed herein, or a pharmaceutically acceptable salt, isotopically enriched analog, stereoisomer, mixture of stereoisomers, or prodrug thereof. The inhibiting can be in vitro or in vivo.

In certain embodiments, provided is a compound as disclosed herein, or a pharmaceutically acceptable salt, isotopically enriched analog, stereoisomer, mixture of stereoisomers, or prodrug thereof, for use in inhibiting SARM1 activity (e.g., in vitro or in vivo).

In certain embodiments, the present disclosure provides use of a compound as disclosed herein, or a pharmaceutically acceptable salt, isotopically enriched analog, stereoisomer, mixture of stereoisomers, or prodrug thereof, in the manufacture of a medicament for inhibiting SARM1 activity (e.g., in vitro or in vivo).

In certain embodiments, provided is a compound as disclosed herein, or a pharmaceutically acceptable salt, isotopically enriched analog, stereoisomer, mixture of stereoisomers, or prodrug thereof, for inhibiting NADase activity of SARM1. In certain embodiments, provided is a method of inhibiting SARM1 NADase activity and/or treating a neurodegenerative or neurological disease or disorder in a subject in need thereof, comprising administering to a subject in need thereof a therapeutically effective amount of a compound disclosed herein, or a pharmaceutically acceptable salt, isotopically enriched analog, stereoisomer, mixture of stereoisomers, or prodrug thereof, to the subject.

In certain embodiments, provided is a method for treating a disease or condition mediated, at least in part, by SARM1, comprising administering to a subject in need thereof a therapeutically effective amount of a compound disclosed herein, or a pharmaceutically acceptable salt, isotopically enriched analog, stereoisomer, mixture of stereoisomers, or prodrug thereof to a subject in need thereof.

In certain embodiments, provided is a method of treating axonal degeneration in a subject in need thereof, comprising administering to a subject in need thereof a therapeutically effective amount of a compound disclosed herein, or a pharmaceutically acceptable salt, isotopically enriched analog, stereoisomer, mixture of stereoisomers, or prodrug thereof, to the subject. In certain embodiments, the compound, or a pharmaceutically acceptable salt, isotopically enriched analog, stereoisomer, mixture of stereoisomers, or prodrug thereof, inhibits axonal degeneration, including axonal degeneration that results from reduction or depletion of NAD+. In certain embodiments, the compound, or a pharmaceutically acceptable salt, isotopically enriched analog, stereoisomer, mixture of stereoisomers, or prodrug thereof, prevents an axon distal to an axonal injury from degenerating.

In certain embodiments, provided is a method for treating degradation of a peripheral nervous system neuron or a portion thereof, comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating degeneration of a central nervous system neuron or a portion thereof, comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, the treating comprises reducing one or more symptoms or features of neurodegeneration.

In certain embodiments, provided is a method for inhibiting axon degeneration, the method comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a neurodegenerative or neurological disease or disorder, the method comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a method for treating a neurodegenerative or neurological disease or disorder associated with axonal degeneration, axonal damage, axonopathy, a demyelinating disease, a central pontine myelinolysis, a nerve injury disease or disorder, a metabolic disease, a mitochondrial disease, metabolic axonal degeneration, axonal damage resulting from traumatic axonal injury (TAI) (see Ziogas et al., J. Neuroscience, 2018, 38(16):4031-4032 and WO2020191257), a leukoencephalopathy or a leukodystrophy, the method comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, provided is a compound as disclosed herein, or a pharmaceutically acceptable salt, isotopically enriched analog, stereoisomer, mixture of stereoisomers, or prodrug thereof, for use in treating a disease or condition mediated, at least in part, by SARM1 in a subject in need thereof.

In certain embodiments, provided is a compound as disclosed herein, or a pharmaceutically acceptable salt, isotopically enriched analog, stereoisomer, mixture of stereoisomers, or prodrug thereof, for use in inhibiting axon degeneration in a subject in need thereof.

In certain embodiments, the present disclosure provides use of a compound as disclosed herein, or a pharmaceutically acceptable salt, isotopically enriched analog, stereoisomer, mixture of stereoisomers, or prodrug thereof in the manufacture of a medicament for inhibiting axon degeneration in a subject in need thereof.

In certain embodiments, the present disclosure provides use of a compound as disclosed herein, or a pharmaceutically acceptable salt, isotopically enriched analog, stereoisomer, mixture of stereoisomers, or prodrug thereof in the manufacture of a medicament for treating a neurodegenerative or neurological disease or disorder, such as a disease or disorder associated with axonal degeneration, axonal damage, axonopathy, a demyelinating disease, a central pontine myelinolysis, a nerve injury disease or disorder, a metabolic disease, a mitochondrial disease, metabolic axonal degeneration, axonal damage resulting from traumatic axonal injury (TAI), a leukoencephalopathy or a leukodystrophy.

In certain embodiments, the disease or condition is an acute condition. In certain embodiments, the disease or condition is a chronic condition.

In certain embodiments, the disease or condition is characterized by axonal degeneration in the central nervous system, the peripheral nervous system, the optic nerve, the cranial nerves, or a combination thereof.

In certain embodiments, the disease or condition is or comprises acute injury to the central nervous system, such as, but not limited to, injury to the spinal cord and/or traumatic brain injury (TBI).

In certain embodiments, the disease or condition is or comprises a chronic injury to the central nervous system, such as, but not limited to, injury to the spinal cord, traumatic brain injury (TBI), and/or traumatic axonal injury (TAI). In certain embodiments, the disease or condition is or comprises chronic traumatic encephalopathy (CTE).

In certain embodiments, the disease or condition is a chronic condition affecting the central nervous system, such as, but not limited to, Parkinson's disease (see, e.g., Sajadi, A., et al. Curr. Biology. 2004, 14, 326-330; and Hasbani, D. M., et al. Exp. Neurology. 2006, 202, 93-99), amyotrophic lateral sclerosis (see, e.g., White, M. A., et al. Acta Neuropath. Comm. 2019, 7(1), 166), multiple sclerosis, Huntington disease, or Alzheimer's disease.

In certain embodiments, the disease or condition is an acute peripheral neuropathy. In certain embodiments, the disease or condition is chemotherapy-induced peripheral neuropathy (CIPN). See, e.g., Geisler, S., et al. Brain. 2016, 139, 3092-3108; Turkiew, E., et al. J. Peripher. Nerv. Syst. 2017, 22, 162-171; Geisler, S., et al. JCI Insight. 2019, 4(17), e129920; and Cetinkaya-Fisgin, A., et al. Sci. Rep. 2020, 21889. Chemotherapy-induced peripheral neuropathy (CIPN), an example of an acute peripheral neuropathy, can be associated with various drugs, such as, but not limited to, thalidomide, epothilones (e.g., ixabepilone), taxanes (e.g., paclitaxel and docetaxel), vinca alkaloids (e.g., vinblastine, vinorelbine, vincristine, and vindesine), proteasome inhibitors (e.g., bortezomib), or platinum-based drugs (e.g., cisplatin, oxaliplatin, and carboplatin).

In certain embodiments, the disease or condition is a chronic condition affecting the peripheral nervous system, such as, but not limited to, diabetic neuropathy, HIV neuropathy, Charcot Marie Tooth disease, or amyotrophic lateral sclerosis.

In certain embodiments, the disease or condition is glaucoma (see, e.g., Ko, K. W., et al. J. Cell Bio. 2020, 219(8), e201912047).

In certain embodiments, the disease or condition is an acute condition affecting the optic nerve, such as, but not limited to, diabetic optic neuropathy, acute optic neuropathy (AON) or acute angle closure glaucoma.

In certain embodiments, the disease or condition is a chronic condition affecting the optic nerve, such as, but not limited to, diabetic optic neuropathy, Leber's congenital amaurosis, Leber's hereditary optic neuropathy (LHON), primary open angle glaucoma, or autosomal dominant optic atrophy.

In certain embodiments, the disease or condition is associated with retinal degeneration. In certain embodiments, the disease or condition is Leber congenital amaurosis, such as Leber congenital amaurosis type 9 (LCA9) (see, e.g., Sasaki, Y., et al. eLife. 2020, 9, e62027.)

In certain embodiments, one or more compounds and/or compositions as described herein are useful, for example, to treat one or more neurodegenerative diseases, disorders or conditions selected from the group consisting of neuropathies or axonopathies. In certain embodiments, one or more compounds and/or compositions as described herein are useful, for example to treat a neuropathy or axonopathy associated with axonal degeneration. In certain embodiments, a neuropathy associated with axonal degeneration is a hereditary or congenital neuropathy or axonopathy. In certain embodiments, a neuropathy associated with axonal degeneration results from a de novo or somatic mutation. In certain embodiments, a neuropathy associated with axonal degeneration is selected from a list contained herein. In certain embodiments, a neuropathy or axonopathy is associated with axonal degeneration, including, but not limited to Parkinson's disease, Alzheimer's disease, herpes infection, diabetes, amyotrophic lateral sclerosis, a demyelinating disease, ischemia, stroke, chemical injury, thermal injury, or AIDS.

In certain embodiments, one or more compounds or compositions as described herein is characterized that, when administered to a population of subjects, reduces one or more symptoms or features of neurodegeneration. For example, in certain embodiments, a relevant symptom or feature may be selected from the group consisting of extent, rate, and/or timing of neuronal disruption. In certain embodiments, neuronal disruption may be or comprise axonal degradation, loss of synapses, loss of dendrites, loss of synaptic density, loss of dendritic arborization, loss of axonal branching, loss of neuronal density, loss of myelination, loss of neuronal cell bodies, loss of synaptic potentiation, loss of action-potential potentiation, loss of cytoskeletal stability, loss of axonal transport, loss of ion channel synthesis and turnover, loss of neurotransmitter synthesis, loss of neurotransmitter release and reuptake capabilities, loss of axon-potential propagation, neuronal hyperexitability, and/or neuronal hypoexcitability. In certain embodiments, neuronal disruption is characterized by an inability to maintain an appropriate resting neuronal membrane potential. In certain embodiments, neuronal disruption is characterized by the appearance of inclusion bodies, plaques, and/or neurofibrillary tangles. In certain embodiments, neuronal disruption is characterized by the appearance of stress granules. In certain embodiments, neuronal disruption is characterized by the intracellular activation of one or more members of the cysteine-aspartic protease (Caspase) family. In certain embodiments, neuronal disruption is characterized by a neuron undergoing programed cell death (e.g. apoptosis, pyroptosis, ferropoptosis, and/or necrosis) and/or inflammation.

In certain embodiments, the neurodegenerative or neurological disease or disorder is associated with axonal degeneration, axonal damage, axonopathy, a demyelinating disease, a central pontine myelinolysis, a nerve injury disease or disorder, a metabolic disease, a mitochondrial disease, metabolic axonal degeneration, axonal damage resulting from a leukoencephalopathy or a leukodystrophy. In certain embodiments, the neurodegenerative or neurological disease or disorder is spinal cord injury, stroke, multiple sclerosis, progressive multifocal leukoencephalopathy, congenital hypomyelination, encephalomyelitis, acute disseminated encephalomyelitis, central pontine myelolysis, osmotic hyponatremia, hypoxic demyelination, ischemic demyelination, adrenoleukodystrophy, Alexander's disease, Niemann-Pick disease, Pelizaeus Merzbacher disease, periventricular leukomalacia, globoid cell leukodystrophy (Krabbe's disease), Wallerian degeneration, optic neuritis, transverse myelitis, amyotrophic lateral sclerosis (ALS, Lou Gehrig's disease), Huntington's disease, Alzheimer's disease, Parkinson's disease, Tay-Sacks disease, Gaucher's disease, Hurler Syndrome, traumatic brain injury (TBI), traumatic axonal injury (TAI), post radiation injury, neurologic complications of chemotherapy (e.g., chemotherapy induced peripheral neuropathy, CIPN), neuropathy, acute ischemic optic neuropathy, vitamin B12 deficiency, isolated vitamin E deficiency syndrome, Bassen-Kornzweig syndrome, retinal degeneration, glaucoma, retinitis pigmentosa, traumatic optic injury, Leber's hereditary optic atrophy (neuropathy), Leber congenital amaurosis (e.g., Leber congenital amaurosis type 9 (LCA9)), neuromyelitis optica, metachromatic leukodystrophy, acute hemorrhagic leukoencephalitis, trigeminal neuralgia, Bell's palsy, cerebral ischemia, multiple system atrophy, traumatic glaucoma, tropical spastic paraparesis human T-lymphotropic virus 1 (HTLV-1) associated myelopathy, west Nile virus encephalopathy, La Crosse virus encephalitis, Bunyavirus encephalitis, pediatric viral encephalitis, essential tremor, Charcot-Marie-Tooth disease, motor neuron disease, spinal muscular atrophy (SMA), hereditary sensory and autonomic neuropathy (HSAN), adrenomyeloneuropathy, progressive supra nuclear palsy (PSP), Friedrich's ataxia, hereditary ataxias, noise induced hearing loss, congenital hearing loss, Lewy Body Dementia, frontotemporal dementia, amyloidosis, diabetic neuropathy, HIV neuropathy, enteric neuropathies and axonopathies, Guillain-Barre syndrome, severe acute motor axonal neuropathy (AMAN), Creutzfeldt-Jakob disease, transmissible spongiform encephalopathy, spinocerebellar ataxias, pre-eclampsia, hereditary spastic paraplegias, spastic paraparesis, familial spastic paraplegia, French settlement disease, Strumpell-Lorrain disease, or non-alcoholic steatohepatitis (NASH).

In certain embodiments, the present disclosure provides inhibitors of SARM1 activity for treatment of neurodegenerative or neurological diseases or disorders that involve axon degeneration or axonopathy. The present disclosure also provides methods of using inhibitors of SARM1 activity to treat, prevent or ameliorate axonal degeneration, axonopathies and neurodegenerative or neurological diseases or disorders that involve axonal degeneration. In certain embodiments, the present disclosure provides a method for inhibiting axon degeneration, the method comprising administering to a subject in need thereof a therapeutically effective amount of a compound disclosed herein, or a pharmaceutically acceptable salt, isotopically enriched analog, stereoisomer, mixture of stereoisomers, or prodrug thereof.

In certain embodiments, the present disclosure provides methods of treating neurodegenerative or neurological diseases or disorders related to axonal degeneration, axonal damage, axonopathies, demyelinating diseases, central pontine myelinolysis, nerve injury diseases or disorders, metabolic diseases, mitochondrial diseases, metabolic axonal degeneration, axonal damage resulting from a leukoencephalopathy or a leukodystrophy.

In certain embodiments, neuropathies and axonopathies include any disease or condition involving neurons and/or supporting cells, such as for example, glia, muscle cells or fibroblasts, and, in particular, those diseases or conditions involving axonal damage. Axonal damage can be caused by traumatic injury or by non-mechanical injury due to diseases, conditions, or exposure to toxic molecules or drugs. The result of such damage can be degeneration or dysfunction of the axon and loss of functional neuronal activity. Disease and conditions producing or associated with such axonal damage are among a large number of neuropathic diseases and conditions. Such neuropathies can include peripheral neuropathies, central neuropathies, or combination thereof. Furthermore, peripheral neuropathic manifestations can be produced by diseases focused primarily in the central nervous systems and central nervous system manifestations can be produced by essentially peripheral or systemic diseases.

In certain embodiments, a peripheral neuropathy may involve damage to the peripheral nerves, and/or can be caused by diseases of the nerves or as the result of systemic illnesses. Some such diseases include diabetes, uremia, infectious diseases such as AIDS or leprosy, nutritional deficiencies, vascular or collagen disorders such as atherosclerosis, or autoimmune diseases such as systemic lupus erythematosus, scleroderma, sarcoidosis, rheumatoid arthritis, and polyarteritis nodosa. In certain embodiments, peripheral nerve degeneration results from traumatic (mechanical) damage to nerves as well as chemical or thermal damage to nerves. Such conditions that injure peripheral nerves include compression or entrapment injuries such as glaucoma, carpal tunnel syndrome, direct trauma, penetrating injuries, contusions, fracture or dislocated bones; pressure involving superficial nerves (ulna, radial, or peroneal) which can result from prolonged use of crutches or staying in one position for too long, or from a tumor; intraneural hemorrhage; ischemia; exposure to cold or radiation or certain medicines or toxic substances such as herbicides or pesticides. In particular, the nerve damage can result from chemical injury due to a cytotoxic anticancer agent such as, for example, taxol, cisplatinin, a proteasome inhibitor, or a vinca alkaloid such as vincristine. Typical symptoms of such peripheral neuropathies include weakness, numbness, paresthesia (abnormal sensations such as burning, tickling, pricking or tingling) and pain in the arms, hands, legs and/or feet. In certain embodiments, a neuropathy is associated with mitochondrial dysfunction. Such neuropathies can exhibit decreased energy levels, i.e., decreased levels of NAD and ATP.

In certain embodiments, peripheral neuropathy is a metabolic and endocrine neuropathy which includes a wide spectrum of peripheral nerve disorders associated with systemic diseases of metabolic origin. These diseases include, for example, diabetes mellitus, hypoglycemia, uremia, hypothyroidism, hepatic failure, polycythemia, amyloidosis, acromegaly, porphyria, a disorder of lipid/glycolipid metabolism, a nutritional/vitamin deficiency, or a mitochondrial disorder. The common hallmark of these diseases is involvement of peripheral nerves by alteration of the structure or function of myelin and axons due to metabolic pathway dysregulation.

In certain embodiments, neuropathies include optic neuropathies such as glaucoma, retinal ganglion degeneration such as those associated with retinitis pigmentosa and outer retinal neuropathies, optic nerve neuritis and/or degeneration including that associated with multiple sclerosis, traumatic injury to the optic nerve which can include, for example, injury during tumor removal, hereditary optic neuropathies such as Kjer's disease and Leber's hereditary optic neuropathy (LHON), ischemic optic neuropathies, such as those secondary to giant cell arteritis, metabolic optic neuropathies such as neurodegenerative diseases including Leber's neuropathy, nutritional deficiencies such as deficiencies in vitamins B12 or folic acid, and toxicities such as due to ethambutol or cyanide, neuropathies caused by adverse drug reactions and neuropathies caused by vitamin deficiency. Ischemic optic neuropathies also include non-arteritic anterior ischemic optic neuropathy.

In certain embodiments, neurodegenerative diseases that are associated with neuropathy or axonopathy in the central nervous system include a variety of diseases. Such diseases include those involving progressive dementia such as, for example, Alzheimer's disease, senile dementia, Pick's disease, and Huntington's disease, central nervous system diseases affecting muscle function such as, for example, Parkinson's disease, motor neuron diseases and progressive ataxias such as amyotrophic lateral sclerosis, demyelinating diseases such as, for example multiple sclerosis, viral encephalitides such as, for example, those caused by enteroviruses, arboviruses, and herpes simplex virus, and prion diseases.

Mechanical injuries such as glaucoma or traumatic injuries to the head and spine can also cause nerve injury and degeneration in the brain and spinal cord. In addition, ischemia and stroke as well as conditions such as nutritional deficiency and chemical toxicity such as with chemotherapeutic agents can cause central nervous system neuropathies.

In certain embodiments, the present disclosure provides a method of treating a neuropathy or axonopathy associated with axonal degeneration. In certain embodiments, a neuropathy or axonopathy associated with axonal degeneration can be any of a number of neuropathies or axonopathies such as, for example, those that are hereditary or congenital or associated with Parkinson's disease, Alzheimer's disease, Herpes infection, diabetes, amyotrophic lateral sclerosis, a demyelinating disease, ischemia or stroke, chemical injury, thermal injury, and AIDS. In addition, neurodegenerative diseases not mentioned above as well as a subset of the above mentioned diseases can also be treated with the methods of the present disclosure. Such subsets of diseases can include Parkinson's disease or Alzheimer's disease.

In certain embodiments, the present methods comprise administering an effective amount of a compound and/or composition as described herein (e.g., a compound of Formula I) to a subject in need thereof. In some such embodiments, the subject is at risk of developing a condition characterized by axonal degeneration. In certain embodiments, the subject has a condition characterized by axonal degeneration. In certain embodiments, the subject has been diagnosed with a condition characterized by axonal degeneration. In certain embodiments, the subject is at risk of developing a condition characterized by axonal degeneration. In certain embodiments, the subject is identified as being at risk of axonal degeneration, e.g., based on the subject's genotype, a diagnosis of a condition associated with axonal degeneration, and/or exposure to an agent and/or a condition that induces axonal degeneration.

In certain embodiments, the subject is at risk of developing a neurodegenerative disorder. In certain embodiments, the subject is elderly. In certain embodiments, the subject is known to have a genetic risk factor for neurodegeneration. In certain embodiments, the subject has a family history of neurodegenerative disease. In certain embodiments, the subject expresses one or more copies of a known genetic risk factor for neurodegeneration. In certain embodiments, the subject is drawn from a population with a high incidence of neurodegeneration. In certain embodiments, the subject has a hexanucleotide repeat expansion in chromosome 9 open reading frame 72. In certain embodiments, the subject has one or more copies of the ApoE4 allele.

In certain embodiments, a neurodegenerative disease, disorder or condition may be or comprise a traumatic neuronal injury. In certain embodiments, a traumatic neuronal injury is blunt force trauma, a closed-head injury, an open head injury, exposure to a concussive and/or explosive force, a penetrating injury in to the brain cavity or innervated region of the body. In certain embodiments, a traumatic neuronal injury is a force which causes the axons to deform, stretch, crush or sheer. In certain embodiments, the disease or disorder is a traumatic brain injury (TBI).

In certain embodiments, the subject has engaged, or engages, in an activity identified as a risk factor for neuronal degradation, e.g., a contact sport or occupations with a high chance for traumatic neuronal injury or TBI.

In certain embodiments, provided is a method of treating a neurodegenerative disease, disorder or condition comprising administering to a patient in need thereof, a compound as described herein, and one or more of a DLK inhibitor or a NAMPT inhibitor. In certain embodiments, provided is a combination therapy comprising a compound as described herein and a DLK inhibitor and/or a NAMPT inhibitor. In certain embodiments, provided is a combination therapy comprising a compound as described herein, a DLK inhibitor, and one or more additional therapeutic agents. In certain embodiments, provided is a combination therapy comprising a compound as described herein, a NAMPT inhibitor, and one or more additional therapeutic agents. In certain embodiments, provided is a combination therapy comprising a compound as described herein, a DLK inhibitor, a NAMPT inhibitor and one or more additional therapeutic agents.

In certain embodiments, the DLK inhibitor is a small molecule, a polypeptide, a peptide fragment, a nucleic acid (e.g., a siRNA, an antisense oligonucleotide, a micro-RNA, or an aptamer), an antibody, a dominant-negative inhibitor, or a ribozyme. In certain embodiments, the DLK inhibitor is a small molecule. In certain embodiments, the DLK inhibitor is a siRNA. In certain embodiments, the DLK inhibitor is an antisense oligonucleotide. In certain embodiments, the DLK inhibitor is a polypeptide. In certain embodiments, a DLK inhibitor is a peptide fragment. In certain embodiments, a DLK inhibitor is a nucleic acid. In certain embodiments, a DLK inhibitor is an antisense oligonucleotide.

Exemplary DLK inhibitors are provided in WO2013174780, WO2014111496, WO2014177524, WO2014177060, WO2015091889, WO2016142310, US20180057507, WO2018107072, WO2019241244, WO2020168111, and CN104387391A, which are hereby incorporated by reference in their entirety.

In certain embodiments, the NAMPT inhibitor is a small molecule, a polypeptide, a peptide fragment, a nucleic acid (e.g., a siRNA, an antisense oligonucleotide, a micro-RNA, or an aptamer), an antibody, a dominant-negative inhibitor, or a ribozyme. In certain embodiments, the NAMPT inhibitor is a small molecule. In some embodiments, the NAMPT inhibitor is a siRNA. In some embodiments, the NAMPT inhibitor is an antisense oligonucleotide. In certain embodiments, the NAMPT inhibitor is a polypeptide. In some embodiments, a NAMPT inhibitor is a peptide fragment. In certain embodiments, a NAMPT inhibitor is a nucleic acid. In some embodiments, a NAMPT inhibitor is an antisense oligonucleotide.

In certain embodiments, a NAMPT inhibitor prevents the formation of nicotinamide mononucleotide (NMN). In certain embodiments, inhibition of NAMPT inhibits the mammalian NAD+ salvage pathway.

In certain embodiments, the provided is a composition comprising a compound as described herein, formulated for use in administering to a subject in combination with a DLK inhibitor and/or a NAMPT inhibitor.

In certain embodiments, the provided is a composition comprising a compound as described herein, for use in combination with a DLK inhibitor and/or a NAMPT inhibitor. In certain embodiments, such compositions are pharmaceutical compositions that include at least one pharmaceutically acceptable carrier, diluent or excipient.

In certain embodiments, the subject may be a subject who has received, is receiving, or has been prescribed, a chemotherapy associated with peripheral neuropathy. Examples of chemotherapeutic agents include, but not limited to, thalidomide, epothilones (e.g., ixabepilone), taxanes (e.g., paclitaxel and docetaxel), vinca alkaloids (e.g., vinblastine, vinorelbine, vincristine, and vindesine), proteasome inhibitors (e.g., bortezomib), platinum-based drugs (e.g., cisplatin, oxaliplatin, and carboplatin).

In certain embodiments, SARM1 inhibition as described herein may be utilized in combination with one or more other therapies to treat a relevant disease, disorder, or condition. In certain embodiments, dosing of a SARM1 inhibitor is altered when utilized in combination therapy as compared with when administered as monotherapy; alternatively or additionally, a therapy that is administered in combination with SARM1 inhibition as described herein is administered according to a regimen or protocol that differs from its regimen or protocol when administered alone or in combination with one or more therapies other than SARM1 inhibition. In certain embodiments, compositions which comprise an additional therapeutic agent, that additional therapeutic agent and a provided compound may act synergistically. In certain embodiments, one or both therapies utilized in a combination regimen is administered at a lower level or less frequently than when it is utilized as monotherapy.

In certain embodiments, a compound, or a pharmaceutically acceptable salt, isotopically enriched analog, stereoisomer, mixture of stereoisomers, or prodrug thereof, or composition provided herein is administered in combination with a NAD⁺ or a NAD⁺ precursor (e.g., nicotinamide riboside (NR), nicotinic acid (NA), nicotinic acid riboside (NaR), nicotinamide (NAM), nicotinamide mononucleotide (NMN), nicotinic acid mononucleotide (NaMN), tryptophan (TRP), nicotinic acid adenine dinucleotide (NAAD), or vitamin B₃).

In certain embodiments, provided is a compound as disclosed herein, or a pharmaceutically acceptable salt, isotopically enriched analog, stereoisomer, mixture of stereoisomers, or prodrug thereof, for use in inhibiting sterile alpha and TIR motif-containing protein 1 (SARM1) activity (e.g., in vitro or in vivo).

In certain embodiments, the present disclosure provides use of a compound as disclosed herein, or a pharmaceutically acceptable salt, isotopically enriched analog, stereoisomer, mixture of stereoisomers, or prodrug thereof, in the manufacture of a medicament for inhibiting sterile alpha and TIR motif-containing protein 1 (SARM1) activity (e.g., in vitro or in vivo) and supplementing axonal NAD⁺ levels.

Axonal degeneration has been associated with various types of neurodegenerative diseases, being recognized as an important indicator of disease progression, and an interesting target for the therapeutic treatment of these diseases. Similarly, axonal degeneration is also observed in those with traumatic brain injuries and peripheral neuropathies.

In certain embodiments, provided is a method for treating a disease or condition mediated, at least in part, by SARM1, comprising administering to a subject in need thereof a therapeutically effective amount of a compound disclosed herein, or a pharmaceutically acceptable salt, isotopically enriched analog, stereoisomer, mixture of stereoisomers, or prodrug thereof, in combination with NAD⁺ or a NAD⁺ precursor (e.g., NR, NA, NaR, NAM, NMN, NaMN, TRP, NAAD, or vitamin B₃).

In certain embodiments, the present disclosure provides use of a compound as disclosed herein, or a pharmaceutically acceptable salt, isotopically enriched analog, stereoisomer, mixture of stereoisomers, or prodrug thereof, in combination with NAD⁺ or a NAD⁺ precursor (e.g., NR, NA, NaR, NAM, NMN, NaMN, TRP, NAAD, or vitamin B₃), in the manufacture of a medicament for treating or preventing a neurodegenerative disease in a subject in need thereof.

In certain embodiments, provided is a method for treating any disease caused by SARM1 activity, comprising administering to a subject in need thereof a therapeutically effective amount of a compound disclosed herein, or a pharmaceutically acceptable salt, isotopically enriched analog, stereoisomer, mixture of stereoisomers, or prodrug thereof, in combination with NAD⁺ or a NAD⁺ precursor (e.g., NR, NA, NaR, NAM, NMN, NaMN, TRP, NAAD, or vitamin B₃).

In certain embodiments, the disease or condition may be a disease or condition of the central nervous system, and/or may be caused by or associated with a pathogen or traumatic injury. It will be appreciated that these general embodiments defined according to broad categories of diseases, disorders and conditions are not mutually exclusive.

In certain embodiments, provided is a method for treating a neurodegenerative disease, comprising administering to a subject in need thereof a therapeutically effective amount of a compound disclosed herein, or a pharmaceutically acceptable salt, isotopically enriched analog, stereoisomer, mixture of stereoisomers, or prodrug thereof, in combination with NAD⁺ or a NAD⁺ precursor (e.g., NR, NA, NaR, NAM, NMN, NaMN, TRP, NAAD, or vitamin B₃).

Other embodiments include use of the presently disclosed compounds in therapy.

### 4. Kits

Provided herein are also kits that include a compound of the disclosure, or a pharmaceutically acceptable salt, isotopically enriched analog, stereoisomer, mixture of stereoisomers, or prodrug thereof, and suitable packaging. In certain embodiments, a kit further includes instructions for use. In one aspect, a kit includes a compound of the disclosure, or a pharmaceutically acceptable salt, isotopically enriched analog, stereoisomer, mixture of stereoisomers, or prodrug thereof, and a label and/or instructions for use of the compounds in the treatment of the indications, including the diseases or conditions, described herein.

Provided herein are also articles of manufacture that include a compound described herein or a pharmaceutically acceptable salt, isotopically enriched analog, stereoisomer, mixture of stereoisomers, or prodrug thereof in a suitable container. The container may be a vial, jar, ampoule, preloaded syringe, or intravenous bag.

### 5. Pharmaceutical Compositions and Modes of Administration

Compounds provided herein are usually administered in the form of pharmaceutical compositions. Thus, provided herein are also pharmaceutical compositions that contain one or more of the compounds described herein, or a pharmaceutically acceptable salt, stereoisomer, mixture of stereoisomers, or prodrug thereof, and one or more pharmaceutically acceptable vehicles selected from carriers, adjuvants, and excipients. Suitable pharmaceutically acceptable vehicles may include, for example, inert solid diluents and fillers, diluents, including sterile aqueous solution and various organic solvents, permeation enhancers, solubilizers, and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G.S. Banker & C.T. Rhodes, Eds.).

The pharmaceutical compositions may be administered in either single or multiple doses. The pharmaceutical composition may be administered by various methods including, for example, rectal, buccal, intranasal, and transdermal routes. In certain embodiments, the pharmaceutical composition may be administered by intra-arterial injection, intravenously, intraperitoneally, parenterally, intramuscularly, subcutaneously, orally, topically, or as an inhalant.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound described herein or a pharmaceutically acceptable salt, isotopically enriched analog, stereoisomer, mixture of stereoisomers, or prodrug thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include, e.g., lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound described herein or a pharmaceutically acceptable salt, isotopically enriched analog, stereoisomer, mixture of stereoisomers, or prodrug thereof can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Another formulation for use in the methods disclosed herein employ transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound described herein or a pharmaceutically acceptable salt, isotopically enriched analog, stereoisomer, mixture of stereoisomers, or prodrug thereof. When referring to these preformulation compositions as homogeneous, the active ingredient may be dispersed evenly throughout the composition so that the composition may be readily subdivided into equally effective unit dosage forms such as tablets, pills, and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described herein. In certain embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, in one embodiment, orally or nasally, from devices that deliver the formulation in an appropriate manner.

The amount of the compound in a pharmaceutical composition or formulation can vary within the full range employed by those skilled in the art. Typically, the formulation will contain, on a weight percent (wt %) basis, from about 0.01-99.99 wt % of a compound of this disclosure based on the total formulation, with the balance being one or more suitable pharmaceutical excipients. In one embodiment, the compound is present at a level of about 1-80 wt %. Representative pharmaceutical formulations are described below.

### Formulation Example 1—Tablet Formulation

The following ingredients are mixed intimately and pressed into single scored tablets.

| | Quantity per |
|---|---|
| Ingredient | tablet, mg |
| compound of this disclosure | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule

| | Quantity per |
|---|---|
| Ingredient | capsule, mg |
| compound of this disclosure | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compound of this disclosure | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| coloring | 0.5 | mg |

| | |
|---|---|
| distilled water | q.s. to 100 mL |

### Formulation Example 4—Injectable Formulation

The following ingredients are mixed to form an injectable formulation.

### Formulation Example 5—Suppository Formulation

| Ingredient | Amount |
|---|---|
| compound of this disclosure | 0.2 mg-20 mg |
| sodium acetate buffer | 2.0 mL |
| solution, 0.4M | |
| HCl (1N) or NaOH (1N) | q.s. to suitable pH |
| water (distilled, sterile) | q.s. to 20 mL |

A suppository of total weight 2.5 g is prepared by mixing the compound of this disclosure with Witepsol® H-15 (triglycerides of saturated vegetable fatty acid; Riches-Nelson, Inc., New York), and has the following composition:

| Ingredient | Amount |
|---|---|
| compound of this disclosure | 500 mg |
| Witepsol ® H-15 | balance |

The specific dose level of a compound of the present application for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound described herein per kilogram of the subject's body weight (mg/kg). Dosages of between about 0.1 and 150 mg/kg may be appropriate. In certain embodiments, about 0.1 and 100 mg/kg may be appropriate. In other embodiments a dosage of between 0.5 and 60 mg/kg may be appropriate. In certain embodiments, a dosage of from about 0.0001 to about 100 mg per kg of body weight per day, from about 0.001 to about 50 mg of compound per kg of body weight, or from about 0.01 to about 10 mg of compound per kg of body weight may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

### 7. Synthesis of the Compounds

The compounds may be prepared using the methods disclosed herein and routine modifications thereof, which will be apparent given the disclosure herein and methods well known in the art.

Conventional and well-known synthetic methods may be used in addition to the teachings herein. The synthesis of typical compounds described herein may be accomplished as described in the following examples. If available, reagents and starting materials may be purchased commercially, e.g., from Sigma Aldrich or other chemical suppliers.

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups (“PG”) may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein. For example, protecting groups for alcohols, such as hydroxy, include silyl ethers (including trimethylsilyl (TMS), tert-butyldimethylsilyl (TBDMS), tri-iso-propylsilyloxymethyl (TOM), and triisopropylsilyl (TIPS) ethers), which can be removed by acid or fluoride ion, such as NaF, TBAF (tetra-n-butylammonium fluoride), HF-Py, or HF-NEt₃. Other protecting groups for alcohols include acetyl, removed by acid or base, benzoyl, removed by acid or base, benzyl, removed by hydrogenation, methoxyethoxymethyl ether, removed by acid, dimethoxytrityl, removed by acid, methoxymethyl ether, removed by acid, tetrahydropyranyl or tetrahydrofuranyl, removed by acid, and trityl, removed by acid. Examples of protecting groups for amines include carbobenzyloxy, removed by hydrogenolysis p-methoxybenzyl carbonyl, removed by hydrogenolysis, tert-butyloxycarbonyl, removed by concentrated strong acid (such as HCl or CF₃COOH), or by heating to greater than about 80° C., 9-fluorenylmethyloxycarbonyl, removed by base, such as piperidine, acetyl, removed by treatment with a base, benzoyl, removed by treatment with a base, benzyl, removed by hydrogenolysis, carbamate group, removed by acid and mild heating, p-methoxybenzyl, removed by hydrogenolysis, 3,4-dimethoxybenzyl, removed by hydrogenolysis, p-methoxyphenyl, removed by ammonium cerium(IV) nitrate, tosyl, removed by concentrated acid (such as HBr or H₂SO₄) and strong reducing agents (sodium in liquid ammonia or sodium naphthalenide), troc (trichloroethyl chloroformate), removed by Zn insertion in the presence of acetic acid, and sulfonamides (Nosyl & Nps), removed by samarium iodide or tributyltin hydride.

Furthermore, the compounds of this disclosure may contain one or more chiral centers. Accordingly, if desired, such compounds can be prepared or isolated as pure stereoisomers, i.e., as individual enantiomers or diastereomers or as stereoisomer-enriched mixtures. All such stereoisomers (and enriched mixtures) are included within the scope of this disclosure, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989) organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### General Synthesis

Scheme I illustrates a general methods which can be employed for the synthesis of compounds described herein (e.g., Formula I), where each X¹, X², X³, X⁴, R, R¹, R⁴, and R⁵ are each independently as defined herein, LG is a leaving group (e.g., halo, alkoxy, —OCCl₃, imidazolyl, 4-nitrobenzyloxy-O—, —S—C₁₋₆ alkyl, —Sn(C₁₋₆ alkyl)₃, etc.), and each R^{5′} are independently —OH, —O-alkyl, or together with the boron atom to which they are attached form a cyclic boronic ester.

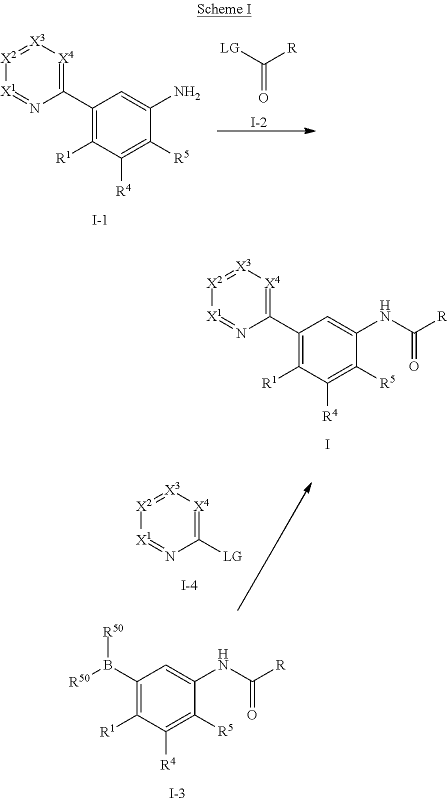

In Scheme I, compounds of Formula I can be prepared by contacting compound I-1 with compound I-2 under suitable coupling reaction conditions, followed by optional functionalization or deprotection when required. Alternatively, compounds of Formula I can be prepared by contacting compound I-3 with compound I-4 under suitable coupling reaction conditions, such as in the presence of a palladium catalyst (e.g., Pd(dppf)Cl₂) and a base, followed by optional functionalization or deprotection when required. Upon each reaction completion, each of the intermediate or final compounds can be recovered, and optionally purified, by conventional techniques such as neutralization, extraction, precipitation, chromatography, filtration and the like.

Further derivatization of the compound provided by the steps outlined in Scheme I, or any intermediate, provides additional compounds of Formula I. It should be understood that any of the compounds or intermediates shown in Scheme I may be prepared using traditional methods or purchased from commercial sources. In addition, any of the intermediates or any product obtained by the process outlined in Scheme I can be derivatized at any step to provide various compounds of Formula I. In certain embodiments, the various substituents of the compounds or intermediates as used in Scheme I are as defined for Formula I.

Scheme II illustrates a general methods which can be employed for the synthesis of compounds described herein (e.g., Formula II), where each X¹, X², X³, X⁴, R¹, R², R³, R⁴, and R⁵ are each independently as defined herein, LG is a leaving group (e.g., halo, alkoxy, —OCCl₃, imidazolyl, 4-nitrobenzyloxy-O—, —S—C₁₋₆ alkyl, —Sn(C₁₋₆ alkyl)₃, etc.), and each R⁵⁰ are independently —OH, —O-alkyl, or together with the boron atom to which they are attached form a cyclic boronic ester.

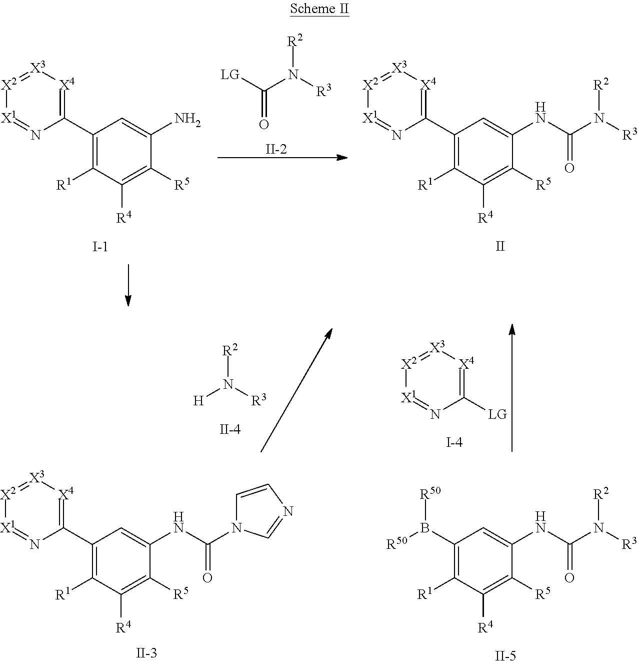

In Scheme II, compounds of Formula II can be prepared by contacting compound I-1 with compound II-2 under suitable coupling reaction conditions, followed by optional functionalization or deprotection when required. Alternatively, compounds of Formula II can be prepared by contacting compound II-3 with compound II-4, or a salt thereof, under suitable coupling reaction conditions, followed by optional functionalization or deprotection when required. Alternatively, compounds of Formula II can be prepared by contacting compound II-5 with compound I-4 under suitable coupling reaction conditions, such as in the presence of a palladium catalyst (e.g., Pd(dppf)Cl₂) and a base, followed by optional functionalization or deprotection when required. Upon each reaction completion, each of the intermediate or final compounds can be recovered, and optionally purified, by conventional techniques such as neutralization, extraction, precipitation, chromatography, filtration and the like.

Further derivatization of the compound provided by the steps outlined in Scheme II, or any intermediate, provides additional compounds of Formula II. It should be understood that any of the compounds or intermediates shown in Scheme I may be prepared using traditional methods or purchased from commercial sources. In addition, any of the intermediates or any product obtained by the process outlined in Scheme II can be derivatized at any step to provide various compounds of Formula II. In certain embodiments, the various substituents of the compounds or intermediates as used in Scheme I are as defined for Formula II.

For example, compounds I-1 and II-5 can be prepared according to Scheme III below according to similar procedures as described in Scheme II, where each X¹, X², X³, X⁴, R¹, R², R³, R⁴, and R⁵ are each independently as defined herein, X is a leaving group (e.g., halo, Sn(C₁₋₆ alkyl)₃, etc.), and each R⁵⁰ are independently —OH, —O-alkyl, or together with the boron atom to which they are attached form a cyclic boronic ester.

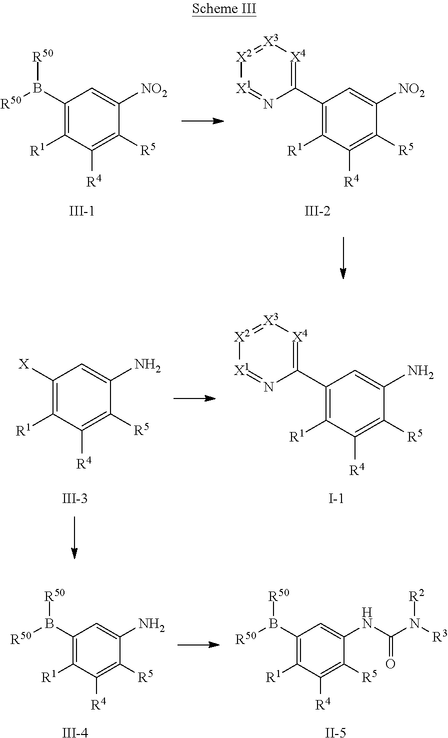

Scheme IV illustrates a general methods which can be employed for the synthesis of compounds described herein (e.g., Formula II and Formula III), where each X¹, X², X³, X⁴, R¹, R², R³, R⁴, R⁵, and R⁷ are each independently as defined herein, each LG is independently a leaving group (e.g., halo, alkoxy, —OCCl₃, imidazolyl, 4-nitrobenzyloxy-O—, —S—C₁₋₆ alkyl, —Sn(C₁₋₆ alkyl)₃, etc.).

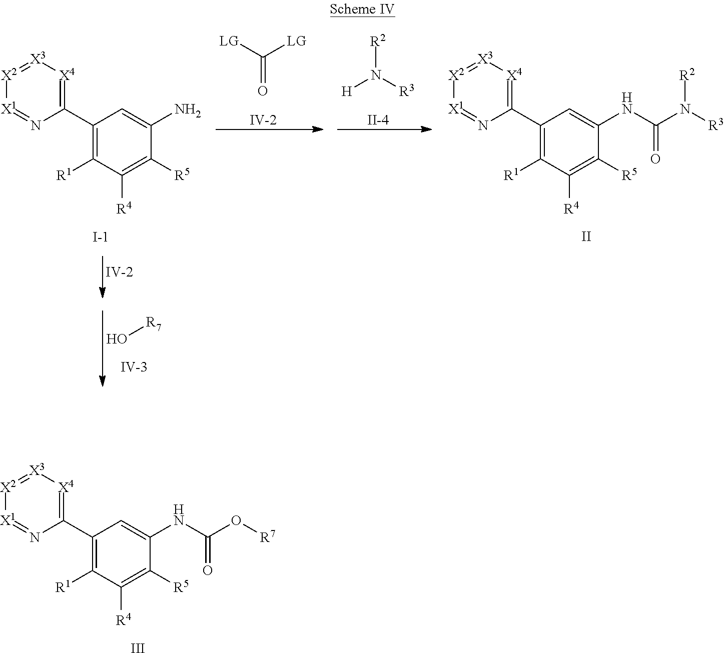

In Scheme IV, compounds of Formula II can be prepared by contacting compound I-1 with compound IV-2 under suitable coupling reaction conditions to provide an acylated intermediate, followed by contacting the acylated intermediate with compound II-4 or a salt thereof. Compounds of Formula III can be prepared by contacting compound I-1 with compound IV-2 under suitable coupling reaction conditions to provide the acylated intermediate, followed by contacting the acylated intermediate with compound IV-3.

In certain embodiments, the acylated intermediate is a compound of Formula IV-1:

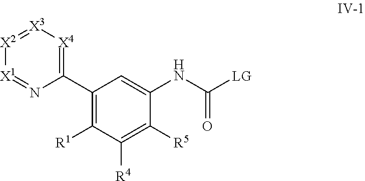

wherein X¹, X², X³, X⁴, R¹, R⁴, R⁵, and LG are each independently as defined herein.

Upon each reaction completion, each of the intermediate or final compounds can be recovered, and optionally purified, by conventional techniques such as neutralization, extraction, precipitation, chromatography, filtration and the like.

It should be understood that any of the compounds or intermediates shown in Scheme II, III, or IV may be prepared using traditional methods or purchased from commercial sources. In addition, any of the intermediates or any product obtained by the process outlined in Scheme II or III can be derivatized at any step to provide various compounds of Formula I. In certain embodiments, the various substituents of the compounds or intermediates as used in Scheme II, III, or IV are as defined for Formula I.

In certain embodiments, provided is a process for providing a compound of Formula I, comprising:
1) contacting a compound of Formula I-1:

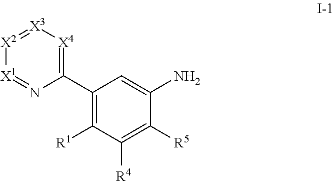

with a compound of Formula IV-2:

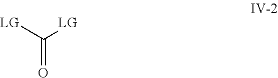

under conditions sufficient to provide an acylated intermediate; and
2) contacting the acylated intermediate with a compound of Formula H—R under conditions sufficient to provide the compound of Formula I; wherein X¹, X², X³, X⁴, R, R¹, R⁴, and R⁵ are each independently as defined herein, and each LG is independently a leaving group. In certain embodiments, each LG is independently selected from halo, alkoxy, —OCCl₃, imidazolyl, 4-nitrobenzyloxy-O—, and —S—C₁₋₆ alkyl.

In certain embodiments, the compound of Formula H—R is —O—R⁷, where R⁷ is as defined herein.

In certain embodiments, the compound of Formula H—R is —NR²R³, where R² and R³ are each independently as defined herein.

In certain embodiments, the acylated intermediate is a compound of Formula IV-1.

In certain embodiments, provided is a process for providing a compound of Formula I, comprising contacting a compound of Formula I-1:

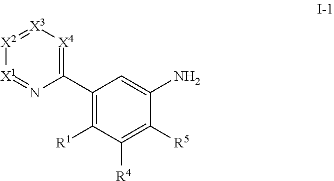

with a compound of Formula I-2:

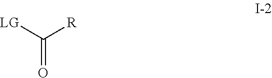

under conditions sufficient to provide a compound of Formula I; wherein X¹, X², X³, X⁴, R, R¹, R⁴, and R⁵ are each independently as defined herein, and LG is a leaving group. In certain embodiments, LG is halo, alkoxy, —OCCl₃, imidazolyl, 4-nitrobenzyloxy-O—, and —S—C₁₋₆ alkyl.

In certain embodiments, provided is a process for providing a compound of Formula I, comprising contacting a compound of Formula I-3:

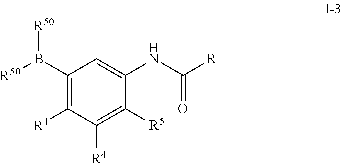

with a compound of Formula I-4:

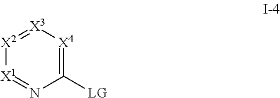

under conditions sufficient to provide a compound of Formula I; wherein X¹, X², X³, X⁴, R, R¹, R⁴, and R⁵ are each independently as defined herein, LG is a leaving group, and each R⁵⁰ is independently —OH, C₁₋₆ alkoxy, or two R⁵⁰ together with the boron atom to which they are attached form a cyclic boronic ester. In certain embodiments, LG is halo, —S—C₁₋₆ alkyl, or —Sn(C₁₋₆ alkyl)₃.

### EXAMPLES

The following examples are included to demonstrate specific embodiments of the disclosure. It should be appreciated by those skilled in the art that the techniques disclosed in the examples which follow represent techniques to function well in the practice of the disclosure, and thus can be considered to constitute specific modes of its practice. However, those of skill in the art should, in light of the present disclosure, appreciate that many changes can be made in the specific embodiments which are disclosed and still obtain a like or similar result without departing from the spirit and scope of the disclosure.

### General Experimental Methods

All solvents used were commercially available and were used without further purification. Reactions were typically run using anhydrous solvents under an inert atmosphere of nitrogen.

NMR Spectroscopy: ¹H Nuclear magnetic resonance (NMR) spectroscopy was carried out using a Bruker Avance III equipped with a BBFO 300 MHz probe operating at 300 MHz or one of the following instruments: a Bruker Avance 400 instrument equipped with probe DUAL 400 MHz S1, a Bruker Avance 400 instrument equipped with probe 6 S1 400 MHz 5 mm ¹H-¹³C ID, a Bruker Avance III 400 instrument with nanobay equipped with probe Broadband BBFO 5 mm direct, a Bruker Mercury Plus 400 NMR spectrometer equipped with a Bruker 400 BBO probe operating at 400 MHz. All deuterated solvents contained typically 0.03% to 0.05% v/v tetramethylsilane, which was used as the reference signal (set at δ 0.00 for both ¹H and ¹³C). In certain cases, ¹H Nuclear magnetic resonance (NMR) spectroscopy was carried out using a Bruker Advance 400 instrument operating at 400 MHz using the stated solvent at around room temperature unless otherwise stated. In all cases, NMR data were consistent with the proposed structures. Characteristic chemical shifts (6) are given in parts-per-million using conventional abbreviations for designation of major peaks: e.g. s, singlet; d, doublet; t, triplet; q, quartet; dd, doublet of doublets; dt, doublet of triplets; br, broad.

Thin Layer Chromatography: Where thin layer chromatography (TLC) has been used it refers to silica gel TLC using silica gel F254 (Merck) plates, Rf is the distance travelled by the compound divided by the distance travelled by the solvent on a TLC plate. Column chromatography was performed using an automatic flash chromatography system over silica gel cartridges or in the case of reverse phase chromatography over C18 cartridges. Alternatively, thin layer chromatography (TLC) was performed on Alugram® (Silica gel 60 F254) from Mancherey-Nagel and UV was typically used to visualize the spots. Additional visualization methods were also employed in some cases. In these cases the TLC plate was developed with iodine (generated by adding approximately 1 g of I₂ to 10 g silica gel and thoroughly mixing), ninhydrin (available commercially from Aldrich), or Magic Stain (generated by thoroughly mixing 25 g (NH₄)₆Mo₇O₂₄·4H₂O, 5 g (NH₄)₂Ce(IV)(NO₃)₆ in 450 mL water and 50 mL concentrated H₂SO₄) to visualize the compound.

Liquid Chromatography-Mass Spectrometry and HPLC Analysis: HPLC analysis was performed on Shimadzu 20AB HPLC system with a photodiode array detector and Luna-C18(2) 2.0×50 mm, 5 μm column at a flow rate of 1.2 mL/min with a gradient solvent Mobile phase A (MPA, H₂O+0.037% (v/v) TFA): Mobile phase B (MPB, ACN+0.018% (v/v) TFA) (0.01 min, 10% MPB; 4 min, 80% MPB; 4.9 min, 80% MPB; 4.92 min, 10% MPB; 5.5 min, 10% MPB). LCMS was detected under 220 and 254 nm or used evaporative light scattering (ELSD) detection as well as positive electrospray ionization (MS). Semi-preparative HPLC was performed by either acidic or neutral conditions. Acidic: Luna C18 100×30 mm, 5 m; MPA: HCl/H₂O=0.04%, or formic acid/H₂O=0.2% (v/v); MPB: ACN. Neutral: Waters Xbridge 150×25, 5 m; MPA: 10 mM NH₄HCO₃ in H₂O; MPB: ACN. Gradient for both conditions: 10% of MPB to 80% of MPB over 12 min at a flow rate of 20 mL/min, then 100% MPB over 2 min, 10% MPB over 2 min, UV detector. SFC analysis was performed on Thar analytical SFC system with a UV/Vis detector and series of chiral columns including AD, AS-H, OJ, OD, AY and IC, 4.6×100 mm, 3 μm column at a flow rate of 4 mL/min with a gradient solvent Mobile phase A (MPA, CO₂): Mobile phase B (MPB, MeOH+0.05% (v/v) IPA) (0.01 min, 10% MPB; 3 min, 40% MPB; 3.5 min, 40% MPB; 3.56-5 min, 10% MPB). SFC preparative was performed on Thar 80 preparative SFC system with a UV/Vis detector and series of chiral preparative columns including AD-H, AS-H, OJ-H, OD-H, AY-H and IC—H, 30×250 mm, 5 m column at a flow rate of 65 mL/min with a gradient solvent Mobile phase A (MPA, CO₂): Mobile phase B (MPB, MeOH+0.1% (v/v) NH₃H₂O) (0.01 min, 10% MPB; 5 min, 40% MPB; 6 min, 40% MPB; 6.1-10 min, 10% MPB). LC-MS data were also collected using an UPLC-MS Acquity™ system equipped with PDA detector and coupled to a Waters single quadrupole mass spectrometer operating in alternated positive and negative electrospray ionization mode. The column used was a Cortecs UPLC C18, 1.6 μm, 2.1×50 mm. A linear gradient was applied, starting at 95% A (A: 0.1% formic acid in water) and ending at 95% B (B: 0.1% formic acid in MeCN) over 2.0 min with a total run time of 2.5 min. The column temperature was at 40° C. with the flow rate of 0.8 mL/min.

### Intermediate 1

### Methyl-6-azabicyclo[3.1.1]heptane

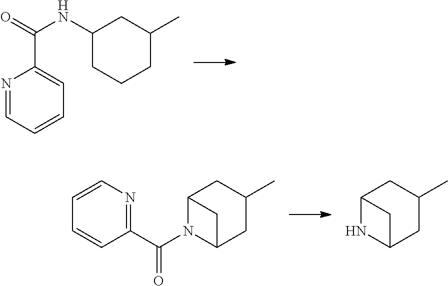

Methyl-6-azabicyclo[3.1.1]heptan-6-yl)(pyridin-2-yl)methanone: To a mixture of N-(3-methylcyclohexyl)picolinamide (20 g, 91.62 mmol) in 1,1,2,2-tetrachloroethane (300 mL) was added AgOAc (45.88 g, 274.86 mmol), benzoquinone (4.95 g, 45.81 mmol), Na₃PO₄ (45 g, 274.86 mmol), 1,2,3,4,5-pentafluoro-6-iodo-benzene (269 g, 916.20 mmol) and Pd(OAc)₂ (2.06 g, 9.16 mmol) at 25° C. under N₂. The mixture was stirred at 145° C. for 12 h. The reaction mixture was filtered through a celite pad and the filtrate was concentrated under reduced pressure. The resulting residue was purified by silica gel column chromatography (PE:EtOAc=5:1 to 1:1) to give the titled compound. LCMS: m/z=217.10 [M+H]⁺.

3-Methyl-6-azabicyclo[3.1.1]heptane: To a mixture of (3-methyl-6-azabicyclo[3.1.1]heptan-6-yl)(pyridin-2-yl)methanone (2.7 g, 12.48 mmol) in EtOH (50 mL) was added NaOH (4.99 g, 124.84 mmol) at 25° C. under N₂. The mixture was stirred at 90° C. for 4 h. The reaction mixture was concentrated under reduced pressure (water pump, below 35° C.) to give a mixture containing the product, NaOH and the sodium salt of the acid. The mixture was stirred in DCM (20 mL) and filtered through a celite pad. The filtrate was concentrated under reduce pressure (water pump, below 35° C.). The work-up procedure was repeated 2-3 times or until the concentrated residue contained no solids to give the titled compound as a 10:1 mixture of cis and trans isomers. LCMS: m/z=112.2 [M+H]⁺.

### Intermediate 2

### trans-3-Methyl-6-azabicyclo[3.1.1]heptane

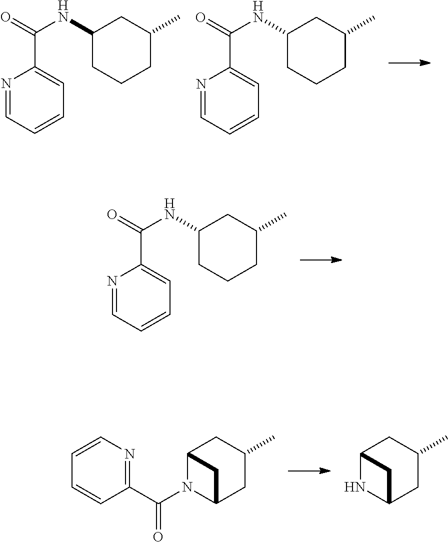

N-(cis-3-methylcyclohexyl)picolinamide: A mixture of N-(trans-3-methylcyclohexyl)picolinamide and N-(cis-3-methylcyclohexyl)picolinamide was purified by prep-HPLC (column: Phenomenex luna C18 250×100 mm×15 m; mobile phase: A: 10 mM TFA in water, B: MeCN; B in A: 40%-70%, over 20 min) to provide the separated cis and trans isomers. The first eluting peak was concentrated under reduced pressure, adjusted to pH=7-8 with sat. aq. NaHCO₃ solution and extracted with DCM (3×500 mL). The combined organic layers were dried over anhydrous Na₂SO₄, filtered and concentrated under reduced pressure to give the titled compound as a colorless oil. LCMS: m/z=219.2 [M+H]⁺.

(trans-3-Methyl-6-azabicyclo[3.1.1]heptan-6-yl)(pyridin-2-yl)methanone To a mixture of N-(cis-3-methylcyclohexyl)picolinamide (10 g, 27.49 mmol) in 1,1,2,2-tetrachloroethane (300 mL) was added AgOAc (22.94 g, 137.43 mmol), benzoquinone (2.48 g, 22.90 mmol), Na₃PO₄ (22.53 g, 137.43 mmol), 1,2,3,4,5-pentafluoro-6-iodo-benzene (134.66 g, 458.10 mmol) and Pd(OAc)₂ (2.06 g, 9.16 mmol) at 25° C. under N₂. The mixture was stirred at 145° C. for 12 h. The reaction mixture was filtered through a celite pad and the filtrate was concentrated under reduced pressure. The resulting residue was purified by silica gel column chromatography (PE:EtOAc=5:1 to 1:1) to give the titled compound as a brown oil. LCMS: m/z=217.0 [M+H]⁺.

trans-3-Methyl-6-azabicyclo[3.1.1]heptane: To a mixture of (trans-3-methyl-6-azabicyclo[3.1.1]heptan-6-yl)(pyridin-2-yl)methanone (1.2 g, 5.55 mmol) in EtOH (15 mL) was added NaOH (2.22 g, 55.48 mmol) at 25° C. under N₂. The mixture was stirred at 90° C. for 4 h. The reaction mixture was concentrated under reduced pressure (water pump, below 35° C.) to give a mixture containing the product, NaOH and the sodium salt of the acid. The mixture was stirred in DCM (20 mL) and filtered through a celite pad. The filtrate was concentrated under reduce pressure (water pump, below 35° C.). The work-up procedure was repeated 2-3 times or until the concentrated residue contained no solids to give the titled compound as a brown syrup as the free base. TFA was added and the mixture was stirred for 0.5 h at 20° C. before concentrating under reduced pressure to give the TFA salt. LCMS: m/z=112.2 [M+H]⁺.

### Intermediate 3

### cis-3-Methyl-6-azabicyclo[3.1.1]heptane

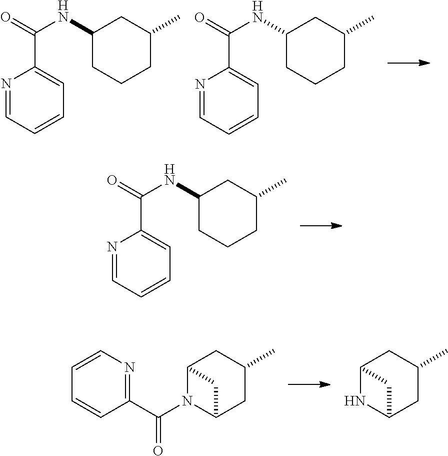

N-(trans-3-methylcyclohexyl)picolinamide: A mixture of N-(trans-3-methylcyclohexyl)picolinamide and N-(cis-3-methylcyclohexyl)picolinamide was purified by prep-HPLC (column: Phenomenex luna C18 250×100 mm×15 m; mobile phase: A: 10 mM TFA in water, B: MeCN; B in A: 40%-70%, over 20 min) to provide the separated cis and trans isomers. The second eluting peak was concentrated under reduced pressure, adjusted to pH=7-8 with sat. aq. NaHCO₃ solution and extracted with DCM (3×300 mL). The combined organic layers were dried over anhydrous Na₂SO₄, filtered and concentrated under reduced pressure to provide the titled compound as a colorless oil. LCMS: m/z=219.2 [M+H]⁺.

(cis-3-Methyl-6-azabicyclo[3.1.1]heptan-6-yl)(pyridin-2-yl)methanone To a mixture of N-(trans-3-methylcyclohexyl)picolinamide (6 g, 27.49 mmol) in 1,1,2,2-tetrachloroethane (200 mL) was added AgOAc (13.76 g, 82.46 mmol), benzoquinone (1.49 g, 13.74 mmol), Na₃PO₄ (13.52 g, 82.46 mmol), 1,2,3,4,5-pentafluoro-6-iodo-benzene (80.80 g, 274.86 mmol) and Pd(OAc)₂ (1.23 g, 5.50 mmol) at 25° C. under N₂. The mixture was stirred at 145° C. for 12 h. The reaction mixture was filtered through a celite pad and the filtrate was concentrated under reduced pressure. The resulting residue was purified by silica gel column chromatography (PE:EtOAc=5:1 to 1:1) to give the titled compound as a brown oil. LCMS: m/z=217.0 [M+H]⁺.

cis-3-Methyl-6-azabicyclo[3.1.1]heptane: To a mixture of (cis-3-methyl-6-azabicyclo[3.1.1]heptan-6-yl)(pyridin-2-yl)methanone (2.4 g, 11.1 mmol) in EtOH (30 mL) was added NaOH (4.44 g, 111 mmol) at 25° C. under N₂. The mixture was stirred at 90° C. for 4 h. The reaction mixture was concentrated under reduced pressure (water pump, below 35° C.) to give a mixture containing the product, NaOH and the sodium salt of the acid. The mixture was stirred in DCM (20 mL) and filtered through a celite pad. The filtrate was concentrated under reduce pressure (water pump, below 35° C.). The work-up procedure was repeated 2-3 times or until the concentrated residue contained no solids to give the titled compound as the free base. TFA was added and the mixture was stirred for 0.5 h at 20° C. before concentrating under reduced pressure to give the TFA salt. LCMS: m/z=112.2 [M+H]⁺.

### Intermediate 4

### 2-methyl-6-azabicyclo[3.1.1]heptane

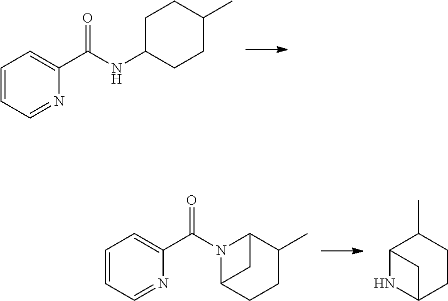

(2-Methyl-6-azabicyclo[3.1.1]heptan-6-yl)(pyridin-2-yl)methanone: To a mixture of N-(4-methylcyclohexyl)picolinamide (5 g, 22.90 mmol) in 1,2-dichloroethane (50 mL) was added AgOAc (11.47 g, 68.71 mmol), benzoquinone (1.24 g, 11.45 mmol), Na₃PO₄ (11.27 g, 68.71 mmol), 1,2,3,4,5-pentafluoro-6-iodo-benzene (67.33 g, 229.05 mmol) and Pd(OAc)₂ (514 mg, 2.29 mmol) at 25° C. under N₂. The mixture was stirred at 140° C. for 12 h. The mixture was filtered and the filtrate was concentrated under reduced pressure. The resulting residue was purified by silica gel column chromatography (PE:EtOAc=10:1 to 1:1) to give the titled compound. LCMS: m/z=217.2 [M+H]⁺.

2-Methyl-6-azabicyclo[3.1.1]heptane: To a mixture of (2-methyl-6-azabicyclo[3.1.1]heptan-6-yl)(pyridin-2-yl)methanone (1 g, 4.62 mmol) in EtOH (15 mL) was added NaOH (1.85 g, 46.24 mmol) at 25° C. under N₂. The mixture was stirred at 90° C. for 12 h. The reaction mixture was concentrated under reduced pressure. The resulting residue was slurried in DCM (10 mL), filtered and the filtrate was concentrated under reduced pressure. The work-up procedure was repeated 2-3 times to give the titled compound. LCMS: m/z=112.2 [M+H]⁺.

### Intermediate 5

### trans-3-Methoxy-6-azabicyclo[3.1.1]heptane

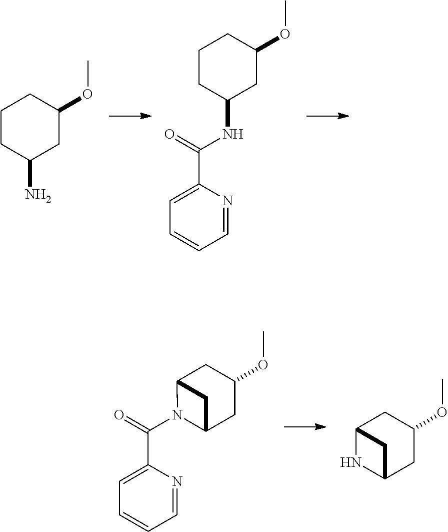

N-(cis-3-Methoxycyclohexyl)picolinamide: To a solution of 3-methoxycyclohexanamine (7.9 g, 47.69 mmol) in DCM (150 mL) was added pyridine-2-carboxylic acid (7.04 g, 57.23 mmol), TEA (14.48 g, 143.06 mmol), DMAP (583 mg, 4.77 mmol) and EDCI (13.71 g, 71.53 mmol) at 0° C. under N₂. The mixture was stirred at 25° C. for 12 h. The mixture was filtered through a celite pad. The filtrate was diluted with H₂O (100 mL), the organic layer was separated, washed with brine (50 mL), dried over anhydrous Na₂SO₄, filtered and concentrated under reduced pressure. The resulting residue was purified by silica gel column chromatography (PE:EtOAc=1:1 to 0:1) to give the titled compound. LCMS: m/z=235.2 [M+H]⁺.

(trans-3-Methoxy-6-azabicyclo[3.1.1]heptan-6-yl)(pyridin-2-yl) methanone: To a solution of N-(3-methoxycyclohexyl)pyridine-2-carboxamide (5.1 g, 21.77 mmol) in 1,1,2,2-tetrachloroethane (150 mL) was added Na₃PO₄ (10.71 g, 65.30 mmol), BQ (1.18 g, 10.88 mmol), AgOAc (10.90 g, 65.30 mmol), 1,2,3,4,5-pentafluoro-6-iodo-benzene (63.99 g, 217.68 mmol) and Pd(OAc)₂ (489 mg, 2.18 mmol) at 25° C. under N₂. The mixture was stirred at 140° C. for 12 h. The mixture was filtered through a celite pad and the filtrate was concentrated under reduced pressure. The resulting residue was purified by silica gel column chromatography (PE:EtOAc=1:1 to 0:1) to give the titled compound. LCMS: m/z=233.1 [M+H]⁺.

trans-3-Methoxy-6-azabicyclo[3.1.1]heptane: To a solution of (trans-3-methoxy-6-azabicyclo[3.1.1]heptan-6-yl)(pyridin-2-yl)methanone (100 mg, 0.43 mmol) in EtOH (2 mL) was added NaOH (172 mg, 4.31 mmol) at 25° C. under N₂. The mixture was stirred at 90° C. for 12 h. The reaction mixture was concentrated under reduced pressure. The reaction mixture was slurried with DCM (10 mL), then filtered through a celite pad, the filtrate was concentrated under reduced pressure to give the titled compound. LCMS: m/z=128.2 [M+H]⁺.

### Intermediate 6

### 3-methyl-3,6-diazabicyclo[3.1.1]heptan-2-one

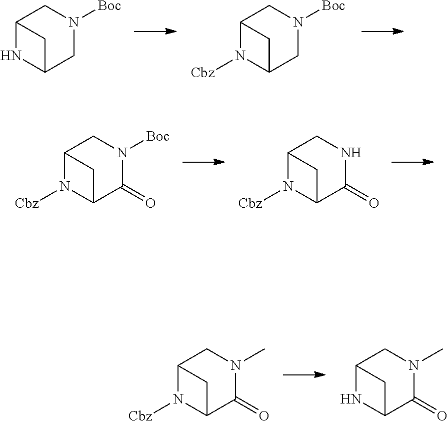

6-Benzyl 3-tert-butyl 3,6-diazabicyclo[3.1.1]heptane-3,6-dicarboxylate: To a solution of tert-butyl 3,6-diazabicyclo[3.1.1]heptane-3-carboxylate (500 mg, 2.52 mmol) in DCM (5 mL) was added TEA (510 mg, 5.04 mmol) followed by the dropwise addition of CbzCl (559 mg, 3.28 mmol) at 0° C. under N₂. The reaction mixture was stirred at 20° C. for 2 h. The reaction mixture was diluted with H₂O (10 mL) and extracted with EtOAc (3×10 mL). The combined organic layers were washed with brine (10 mL), dried over anhydrous Na₂SO₄, filtered and concentrated under reduced pressure. The resulting residue was purified by silica gel column chromatography (PE:EtOAc=1:0 to 1:1) to give the titled compound. LCMS: m/z=233.1 [M-Boc+H]⁺.

6-Benzyl 3-tert-butyl 2-oxo-3,6-diazabicyclo[3.1.1]heptane-3,6-dicarboxylate: To a solution of 6-benzyl 3-tert-butyl 3,6-diazabicyclo[3.1.1]heptane-3,6-dicarboxylate (2.1 g, 6.32 mmol) in EtOAc (12 mL) and H₂O (4 mL) was added RuCl₃ (655 mg, 3.16 mmol) and NaIO₄ (4.05 g, 18.95 mmol) at 0° C. under N₂. The mixture was stirred at 20° C. for 2 h. The reaction mixture was diluted with H₂O (20 mL) and extracted with EtOAc (3×15 mL). The combined organic layers were washed with brine (15 mL), dried over anhydrous Na₂SO₄, filtered and concentrated under reduced pressure. The resulting residue was purified by silica gel column chromatography (PE:EtOAc=10:1 to 3:1) to give the titled compound. LCMS: m/z=247.0 [M-Boc+H]⁺.

Benzyl 2-oxo-3,6-diazabicyclo[3.1.1]heptane-6-carboxylate: To a solution of 6-benzyl 3-tert-butyl 2-oxo-3,6-diazabicyclo[3.1.1]heptane-3,6-dicarboxylate (1.4 g, 4.04 mmol) in EtOAc (5 mL) was added HCl/EtOAc (12 mL, 4 M) at 20° C. The mixture was stirred at 20° C. for 1 h and was then concentrated under reduced pressure to give the titled compound. LCMS: m/z=247.0 [M+H]⁺.

Benzyl 3-methyl-2-oxo-3,6-diazabicyclo[3.1.1]heptane-6-carboxylate: To a solution of benzyl 2-oxo-3,6-diazabicyclo[3.1.1]heptane-6-carboxylate (700 mg, 2.84 mmol) in THF (8 mL) was added NaH (159 mg, 3.98 mmol, 60% purity) and MeI (605 mg, 4.26 mmol, 0.26 mL) at 0° C. under N₂. The mixture was stirred at 20° C. for 2 h. The reaction mixture was quenched by the addition of H₂O (10 mL) and extracted with EtOAc (3×10 mL). The combined organic layers were washed with brine (10 mL), dried over anhydrous Na₂SO₄, filtered and concentrated under reduced pressure. The resulting residue was purified by silica gel column chromatography (PE:EtOAc=1:0 to 0:1) to give the titled compound. LCMS: m/z=261.0 [M+H]⁺.

3-Methyl-3,6-diazabicyclo[3.1.1]heptan-2-one: To a solution of benzyl 3-methyl-2-oxo-3,6-diazabicyclo[3.1.1]heptane-6-carboxylate (450 mg, 1.73 mmol) in MeOH (8 mL) was added Pd/C (450 mg, 10% on carbon) under N₂. The suspension was degassed under vacuum and purged with H₂ several times. The mixture was stirred under H₂ (15 psi) at 20° C. for 2 h. The reaction mixture was filtered through a celite pad and the filtrate was concentrated under reduced pressure to give the titled compound. LCMS: m/z=127.0 [M+H]⁺.

### Intermediate 7

### 2,3,4,5-tetrahydro-2,5-methanobenzo[f][1,4]oxazepine

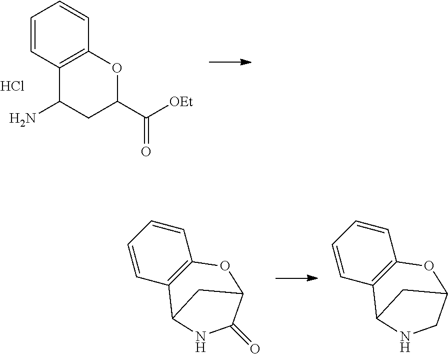 [SMILES: Cl ; CCOC(=O)C1CC(N)c2ccccc2O1 ; O=C1NC2CC1Oc1ccccc12 ; c1ccc2c(c1)OC1CNC2C1]

4,5-dihydro-2,5-methanobenzo[f][1,4]oxazepin-3(2H)-one: To a mixture of ethyl 4-aminochroman-2-carboxylate hydrochloride (900 mg, 3.49 mmol) in toluene (30 mL) was added TEA (1.06 g, 10.48 mmol) at 20° C. under N₂. The reaction mixture was stirred at 110° C. for 16 h. The mixture was concentrated under reduced pressure and the resulting residue was diluted with H₂O (10 mL) and extracted with EtOAc (3×10 mL). The combined organic layers were dried over anhydrous Na₂SO₄, filtered, and concentrated under reduced pressure. The resulting residue was triturated with MTBE, collected by filtration and the solid was dried under reduced pressure to give the titled compound. LCMS: m/z=176.1 [M+H]⁺.

2,3,4,5-tetrahydro-2,5-methanobenzo[f][1,4]oxazepine: To a mixture of 4,5-dihydro-2,5-methanobenzo[f][1,4]oxazepin-3(2H)-one (395 mg, 2.25 mmol) in THF (15 mL) was added LiAlH₄ (428 mg, 11.27 mmol) at 0° C. under N₂. The mixture was stirred at 70° C. for 16 h. The mixture was quenched with Na₂SO₄·10H₂O until bubbling ceased and was stirred for 30 min. The mixture was filtered through a celite pad and the filtrate was concentrated under reduced pressure to give the titled compound. LCMS: m/z=162.2 [M+H]⁺.

### Intermediate 8

### cis-3-Fluoro-6-azabicyclo[3.1.1]heptane trifluoroacetate

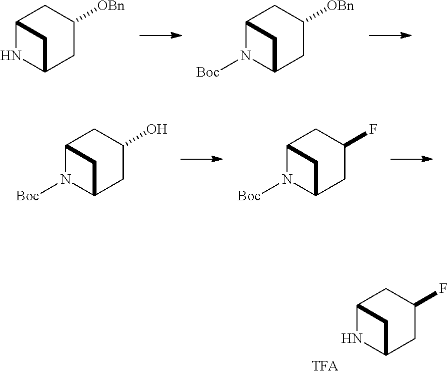

trans-tert-Butyl 3-(benzyloxy)-6-azabicyclo[3.1.1]heptane-6-carboxylate: To a solution of trans-3-(benzyloxy)-6-azabicyclo[3.1.1]heptane (5.8 g, 28.53 mmol) in 1,4-dioxane (20 mL) was added aq. NaOH (50 mL, 2 M) and Boc₂O (12.45 g, 57.06 mmol) at 0° C. The mixture was stirred at 20° C. for 12 h. The reaction mixture was diluted with H₂O (50 mL) and extracted with MTBE (3×50 mL). The combined organic layers were washed with brine (50 mL), dried over anhydrous Na₂SO₄, filtered and concentrated under reduced pressure. The resulting residue was purified by silica gel column chromatography (PE:MTBE=100:1 to 3:1) to give the titled compound. LCMS: m/z=248.2 [M-tBu+H]⁺.

trans-tert-Butyl 3-hydroxy-6-azabicyclo[3.1.1]heptane-6-carboxylate: To a solution of trans-tert-butyl 3-(benzyloxy)-6-azabicyclo[3.1.1]heptane-6-carboxylate (3.55 g, 11.70 mmol) in MeOH (50 mL) was added Pd/C (2 g, 10% on carbon) at 25° C. under Ar. The suspension was degassed and purged with H₂ three times. The mixture was stirred at 30° C. for 12 h under H₂ (50 psi). The reaction mixture was filtered through a celite pad and the filtrate was concentrated under reduced pressure to give the titled compound. LCMS: m/z=158.2 [M-tBu+H]⁺.

cis-tert-Butyl 3-fluoro-6-azabicyclo[3.1.1]heptane-6-carboxylate: To a solution of trans-tert-butyl 3-hydroxy-6-azabicyclo[3.1.1]heptane-6-carboxylate (110 mg, 0.09 mmol) in DCM (2 mL) was added DAST (166 mg, 0.19 mmol) at 0° C. under N₂. The mixture was stirred at 0° C. for 2 h. The reaction mixture was adjusted to pH=7-8 with sat. aq. NaHCO₃ and extracted with DCM (3×5 mL). The combined organic layers were dried over anhydrous Na₂SO₄, filtered and concentrated under reduced pressure to give the titled compound. LCMS: m/z=160.2 [M-tBu+H]⁺.

cis-3-Fluoro-6-azabicyclo[3.1.1]heptane trifluoroacetate: To a solution of cis-tert-butyl 3-fluoro-6-azabicyclo[3.1.1]heptane-6-carboxylate (30 mg, 0.14 mmol) in DCM (3 mL) was added TFA (1.54 g, 14 mmol, 1 mL) at 20° C. The mixture was stirred at 20° C. for 2 h. The reaction mixture was concentrated under reduced pressure to give the titled compound. The material was used directly in the next step.

### Intermediate 9

### trans-3-(Difluoromethoxy)-6-azabicyclo[3.1.1]heptane trifluoroacetate

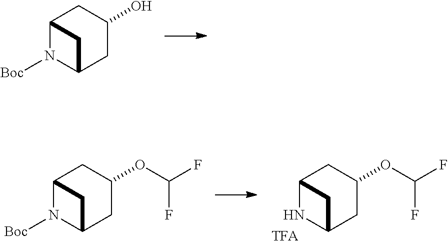

trans-tert-Butyl 3-(difluoromethoxy)-6-azabicyclo[3.1.1]heptane-6-carboxylate: To a solution of trans-tert-butyl 3-hydroxy-6-azabicyclo[3.1.1]heptane-6-carboxylate (100 mg, 0.47 mmol) in DCM (1.5 mL) and H₂O (1.5 mL) was added KHF₂ (220 mg, 2.81 mmol) and (bromodifluoromethyl)trimethylsilane (286 mg, 1.41 mmol) at 20° C. The mixture was stirred at 20° C. for 12 h. The reaction mixture was diluted with H₂O (3 mL) and extracted with DCM (3×5 mL). The combined organic layers were washed with brine (5 mL), dried over anhydrous Na₂SO₄, filtered and concentrated under reduced pressure to give the titled compound. LCMS: m/z=208.1 [M-tBu]⁺.

trans-3-(Difluoromethoxy)-6-azabicyclo[3.1.1]heptane trifluoroacetate: To a solution of trans-tert-butyl 3-(difluoromethoxy)-6-azabicyclo[3.1.1]heptane-6-carboxylate (75 mg, 0.28 mmol) in DCM (1.5 mL) was added TFA (2.3 g, 20.26 mmol, 1.50 mL) at 20° C. The mixture was stirred at 20° C. for 2 h. The reaction mixture was concentrated under reduced pressure to give the titled compound. LCMS: m/z=164.2 [M+H]⁺.

### Intermediate 10

### cis-3-Methoxy-6-azabicyclo[3.1.1]heptane

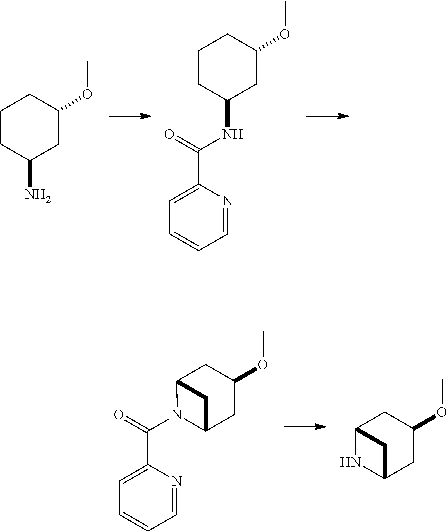

N-(trans-3-methoxycyclohexyl)picolinamide: To a mixture of trans-3-methoxycyclohexanamine hydrochloride (2 g, 12.07 mmol) and picolinic acid (1.78 g, 14.49 mmol) in DCM (50 mL) was added TEA (1.83 g, 18.11 mmol), DMAP (147.49 mg, 1.21 mmol) and EDCI (3.47 g, 18.11 mmol) at 0° C. under N₂. The mixture was stirred at 25° C. for 16 h. The reaction mixture was diluted with H₂O (20 mL), the organic layer was separated and the aqueous layer was extracted with DCM (3×10 mL). The combined organic layers were washed with brine (20 mL), dried over anhydrous Na₂SO₄, filtered and concentrated under reduced pressure. The resulting residue was purified by silica gel column chromatography (PE:EtOAc=5:1 to 3:1) to give the titled compound. LCMS: m/z=235.1 [M+H]⁺.

(cis-3-methoxy-6-azabicyclo[3.1.1]heptan-6-yl)(pyridin-2-yl)methanone: To a mixture of N-((1S,3S)-3-methoxycyclohexyl)picolinamide (1.6 g, 6.83 mmol) in 1,1,2,2-tetrachloroethane (50 mL) was added AgOAc (3.42 g, 20.49 mmol), benzoquinone (369 mg, 3.41 mmol), Na₃PO₄ (3.36 g, 20.49 mmol), 1,2,3,4,5-pentafluoro-6-iodo-benzene (20.07 g, 68.29 mmol) and Pd(OAc)₂ (307 mg, 1.37 mmol) at 25° C. under N₂. The mixture was stirred at 140° C. for 12 h. The reaction mixture was filtered through a celite pad and the filtrate was concentrated under reduced pressure. The resulting residue was purified by silica gel column chromatography (PE:EtOAc=5:1 to 1:1) to give the titled compound. LCMS: m/z=232.9 [M+H]⁺.

cis-3-Methoxy-6-azabicyclo[3.1.1]heptane: To a mixture of (cis-3-methoxy-6-azabicyclo[3.1.1]heptan-6-yl)(pyridin-2-yl)methanone (270 mg, 1.16 mmol) in EtOH (5 mL) was added NaOH (465 mg, 11.62 mmol) at 25° C. The mixture was stirred at 90° C. for 4 h before concentrating under reduced pressure. The resulting residue was slurried in DCM (20 mL), filtered and the filtrate was concentrated under reduce pressure. The work up was repeated for three times to give the titled compound. The material was used directly in the next step.

### Intermediate 11

### trans-1,6,7-trimethyl-3-azabicyclo[4.1.0]heptane hydrochloride

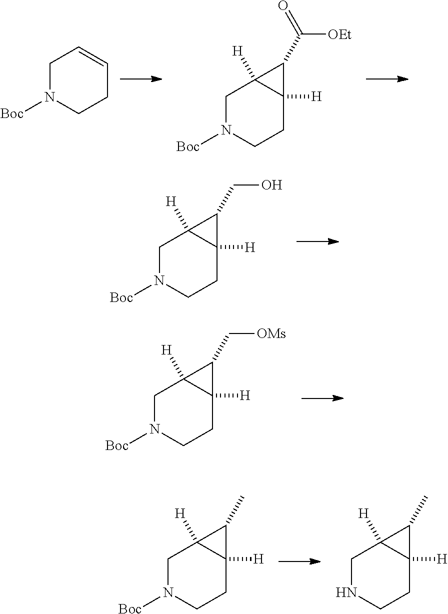

trans-3-tert-butyl 7-ethyl 3-azabicyclo[4.1.0]heptane-3,7-dicarboxylate: To a mixture of tert-butyl 5,6-dihydropyridine-1(2H)-carboxylate (10 g, 54.57 mmol) and R^{h}(OAc)₂ (603 mg, 2.73 mmol) in DCE (300 mL) was added dropwise ethyl 2-diazoacetate (18.68 g, 163.71 mmol) in DCE (200 mL) at 80° C. under N₂ and stirred at 80° C. for 16 h. The mixture was filtered through a celite pad and the filtrate was concentrated under reduced pressure. The resulting residue was purified by silica gel column chromatography (PE:EtOAc=10:1 to 8:1) to give the titled compound.

trans-tert-butyl 7-(hydroxymethyl)-3-azabicyclo[4.1.0]heptane-3-carboxylate: To a mixture of trans-3-tert-butyl 7-ethyl 3-azabicyclo[4.1.0]heptane-3,7-dicarboxylate (3 g, 11.14 mmol) in DCM (60 mL) was added DIBAL-H (22.28 mmol, 1 M in Tol., 22.28 mL) at 0° C. under N₂ and stirred at 25° C. for 16 h. The mixture was diluted with H₂O (20 mL), potassium sodium tartrate (10 g) was added and the mixture was stirred for 30 min. The mixture was filtered through a celite pad and the filtrate was extracted with DCM (3×20 mL). The combined organic layers were dried over anhydrous Na₂SO₄, filtered and concentrated under reduced pressure. The resulting residue was purified by silica gel column chromatography (PE:EtOAc=3:1 to 1:1) to give the titled compound.

trans-tert-butyl 7-(((methylsulfonyl)oxy)methyl)-3-azabicyclo[4.1.0]heptane-3-carboxylate: To a mixture of trans-tert-butyl 7-(hydroxymethyl)-3-azabicyclo[4.1.0]heptane-3-carboxylate (562 mg, 2.47 mmol) and TEA (500 mg, 4.95 mmol, 0.69 mL) in DCM (10 mL) was added MsCl (312 mg, 2.72 mmol, 0.21 mL) at 0° C. under N₂ and stirred at 25° C. for 30 min. The mixture was poured into water (15 mL) and extracted with DCM (3×5 mL). The combined organic layers were washed with sat. aq. NaHCO₃ (5 mL), dried over anhydrous Na₂SO₄, filtered and concentrated under reduced pressure to give the titled compound.

trans-tert-butyl 7-methyl-3-azabicyclo[4.1.0]heptane-3-carboxylate: To a mixture of trans-tert-butyl 7-(((methylsulfonyl)oxy)methyl)-3-azabicyclo[4.1.0]heptane-3-carboxylate (559 mg, 1.83 mmol) in THF (15 mL) was added LiBHEt₃ (3.66 mmol, 1 M in THF, 3.66 mL) at 0° C. under N₂ and stirred at 25° C. for 16 h. The mixture was poured into sat. aq. NH₄Cl (15 mL) and extracted with EtOAc (3×5 mL). The combined organic layers were dried over anhydrous Na₂SO₄, filtered and concentrated under reduced pressure. The resulting residue was purified by silica gel column chromatography (PE:EtOAc=7:1 to 5:1) to give the titled compound.

trans-7-methyl-3-azabicyclo[4.1.0]heptane hydrochloride: A solution of trans-tert-butyl 7-methyl-3-azabicyclo[4.1.0]heptane-3-carboxylate (60 mg, 0.28 mmol) in HCl/EtOAc (20 mmol, 4 M, 5 mL) was stirred at 25° C. for 2 h. The mixture was concentrated under reduced pressure to give the titled compound.

### Intermediate 12

### trans-6-Azabicyclo[3.1.1]heptan-3-ol

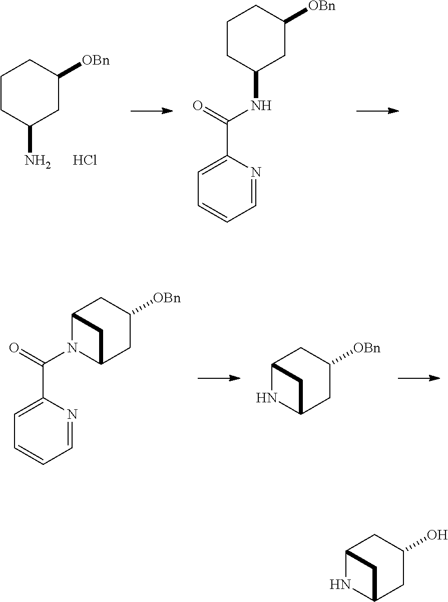 [SMILES: N[C@H]1CCC[C@@H](OCc2ccccc2)C1 ; Cl ; O=C(N[C@H]1CCC[C@@H](OCc2ccccc2)C1)c1ccccn1 ; O=C(c1ccccn1)N1[C@@H]2C[C@H](OCc3ccccc3)C[C@H]1C2 ; c1ccc(CO[C@@H]2C[C@H]3C[C@@H](C2)N3)cc1 ; O[C@H]1C[C@@H]2C[C@H](C1)N2]

N-(cis-3-(benzyloxy)cyclohexyl)picolinamide: To a mixture of cis-3-(benzyloxy)cyclohexanamine hydrochloride (61.5 g, 254.39 mmol) and picolinic acid (37.58 g, 305.27 mmol) in EtOAc (400 mL) was added TEA (102.97 g, 1.02 mol, 141.63 mL), followed by T3P (242.82 g, 381.58 mmol, 50% solution in EtOAc) at 0° C. under N₂. The mixture was stirred at 25° C. for 12 h. The reaction mixture was diluted with H₂O (1 L) and extracted with EtOAc (3×300 mL). The combined organic layers were dried over anhydrous Na₂SO₄, filtered and concentrated under reduced pressure. The resulting residue was purified by silica gel column chromatography (PE:EtOAc=3:1 to 1:1) to give the titled compound. LCMS: m/z=311.0 [M+H]⁺.

(trans-3-(Benzyloxy)-6-azabicyclo[3.1.1]heptan-6-yl)(pyridin-2-yl)methanone: To a solution of N-(cis-3-(benzyloxy)cyclohexyl)picolinamide (39 g, 125.58 mmol) in 1,1,2,2-tetrachloroethane (500 mL) was added benzoquinone (6.79 g, 62.79 mmol), AgOAc (62.79 g, 376.74 mmol), Na₃PO₄ (61.62 g, 376.74 mmol), 1,2,3,4,5-pentafluoro-6-iodo-benzene (369.33 g, 1.26 mol) and Pd(OAc)₂ (2.81 g, 12.48 mmol) at 20° C. under N₂. The mixture was stirred at 140° C. for 12 h. The reaction mixture was filtered through a celite pad and the filtrate was concentrated under reduced pressure. The resulting residue was purified by silica gel column chromatography (EtOAc) to give the titled compound. The material was used directly in the next step.

trans-3-(Benzyloxy)-6-azabicyclo[3.1.1]heptane: To a mixture of (trans-3-(benzyloxy)-6-azabicyclo[3.1.1]heptan-6-yl)(pyridin-2-yl)methanone (13 g, 42.16 mmol) in EtOH (130 mL) was added NaOH (16.86 g, 421.57 mmol) at 20° C. under N₂. The mixture was stirred at 90° C. for 12 h. The reaction mixture was concentrated under reduced pressure, slurried in DCM (100 mL), filtered through a celite pad and the filtrate was concentrated under reduced pressure. This workup was repeated for 2-3 times to give the titled compound. LCMS: m/z=204.3 [M+H]⁺.

trans-6-Azabicyclo[3.1.1]heptan-3-ol: To a solution of trans-3-(benzyloxy)-6-azabicyclo[3.1.1]heptane (200 mg, 0.98 mmol) in MeOH (20 mL) was added Pd/C (50 mg, 10% purity) at 25° C. under N₂. The suspension was degassed and purged with H₂ for 3 times. The mixture was stirred under H₂ (50 psi) at 30° C. for 12 h. The mixture was filtered through a celite pad and the filtrate was concentrated under reduced pressure to give the titled compound. LCMS: m/z=114.2 [M+H]⁺.

### Intermediate 13

### 4-Fluoro-5,6,7,8-tetrahydro-5,8-epiminoisoquinoline hydrochloride

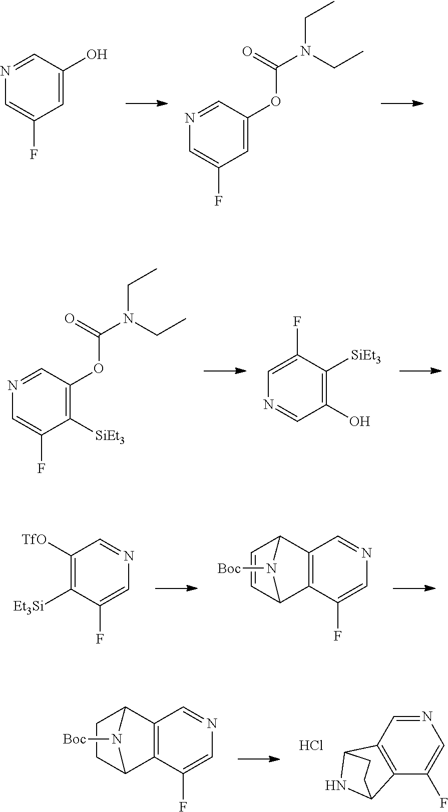

5-Fluoropyridin-3-yl diethylcarbamate: To a mixture of 5-fluoropyridin-3-ol (40 g, 353.70 mmol) in toluene (400 mL) was added TEA (42.95 g, 424.44 mmol) at 0° C. and stirred for 0.5 h, then diethylcarbamic chloride (57.55 g, 424.44 mmol) in toluene (120 mL) was added to the mixture at 0° C. under N₂. The mixture was stirred at 120° C. for 2 h. The reaction mixture was filtered and the filtrate was concentrated under reduced pressure. The resulting residue was purified by silica gel column chromatography (PE:EtOAc=5:1 to 1:1) to give the titled compound. LCMS: m/z=213.0 [M+H]⁺.

5-Fluoro-4-(triethylsilyl)pyridin-3-yl diethylcarbamate: To a mixture of 5-fluoropyridin-3-yl diethylcarbamate (10 g, 47.12 mmol) in THF (100 mL) was added dropwise LDA (25.92 mL, 2 M in THF) at −78° C. under N₂. The mixture was stirred at −78° C. for 30 min, then chloro(triethyl)silane (7.81 g, 51.83 mmol, 8.82 mL) was added to the mixture at −78° C. The reaction mixture was stirred at 25° C. for another 2 h. The reaction mixture was poured into sat. aq. NH₄Cl (50 mL) and extracted with EtOAc (3×30 mL). The combined organic layers were washed with brine (30 mL), dried over anhydrous Na₂SO₄, filtered and concentrated under reduced pressure. The resulting residue was purified by silica gel column chromatography (PE:MTBE=10:1 to 3:1) to give the titled compound. LCMS: m/z=327.1 [M+H]⁺.

5-Fluoro-4-(triethylsilyl)pyridin-3-ol: To a mixture of 5-fluoro-4-(triethylsilyl)pyridin-3-yl diethylcarbamate (2 g, 6.13 mmol) in THF (40 mL) was added LiAlH₄ (581 mg, 15.31 mmol) at 0° C. under N₂. The mixture was stirred at 25° C. for 2 h. The reaction mixture was added with Na₂SO₄·10H₂O at 0° C., the mixture was filtered through a celite pad. The filtrate was concentrated under reduced pressure. The resulting residue was purified by silica gel column chromatography (PE:EtOAc=2:1 to 1:1) to give the titled compound. LCMS: m/z=228.0 [M+H]⁺.

5-Fluoro-4-(triethylsilyl)pyridin-3-yl trifluoromethanesulfonate: To a mixture of 5-fluoro-4-(triethylsilyl)pyridin-3-ol (1.4 g, 6.16 mmol) in pyridine (20 mL) was added Tf₂O (3.82 g, 13.54 mmol) at 0° C. under N₂. The mixture was stirred at 25° C. for 12 h. The reaction mixture was diluted with H₂O (20 mL) and extracted with EtOAc (3×20 mL). The combined organic layers were dried over anhydrous Na₂SO₄, filtered and concentrated under reduced pressure. The resulting residue was purified by silica gel column chromatography (PE:EtOAc=5:1 to 1:1) to give the titled compound. LCMS: m/z=360.0 [M+H]⁺.

tert-Butyl 4-fluoro-5,8-dihydro-5,8-epiminoisoquinoline-9-carboxylate: To a mixture of 5-fluoro-4-(triethylsilyl)pyridin-3-yl trifluoromethanesulfonate (1.15 g, 3.20 mmol) in MeCN (25 mL) was added CsF (972 mg, 6.40 mmol) and tert-butyl 1H-pyrrole-1-carboxylate (2.67 g, 16.00 mmol) at 25° C. under N₂. The mixture was stirred at 25° C. for 4 h. The reaction mixture was diluted with H₂O (10 mL) and extracted with EtOAc (3×15 mL). The combined organic layers were dried over anhydrous Na₂SO₄, filtered and concentrated under reduced pressure. The resulting residue was purified by silica gel column chromatography (PE:EtOAc=3:1 to 1:1) to give the titled compound. LCMS: m/z=263.0 [M+H]⁺.

tert-Butyl 4-fluoro-5,6,7,8-tetrahydro-5,8-epiminoisoquinoline-9-carboxylate: To a solution of tert-butyl 4-fluoro-5,8-dihydro-5,8-epiminoisoquinoline-9-carboxylate (750 mg, 2.86 mmol) in EtOAc (10 mL) was added Pd/C (300 mg, 10% purity) under H₂. The suspension was degassed under vacuum and purged with H₂ several times. The mixture was stirred under H₂ (30 psi) at 25° C. for 12 h. The reaction mixture was filtered through a celite pad. The filtrate was concentrated under reduced pressure to give the titled compound. LCMS: m/z=265.1 [M+H]⁺.

4-Fluoro-5,6,7,8-tetrahydro-5,8-epiminoisoquinoline hydrochloride: To a mixture of tert-butyl 4-fluoro-5,6,7,8-tetrahydro-5,8-epiminoisoquinoline-9-carboxylate (700 mg, 2.65 mmol) in EtOAc (2 mL) was added HCl/EtOAc (10 mL, 4 M) at 25° C. under N₂. The mixture was stirred at 25° C. for 2 h. The reaction mixture was concentrated under reduced pressure to give the titled compound. The material was used directly in the next step.

### Intermediate 14

### 4-Methyl-3-(pyridin-2-yl)aniline

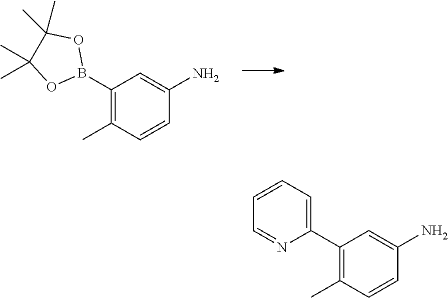 [SMILES: Cc1ccc(N)cc1B1OC(C)(C)C(C)(C)O1 ; Cc1ccc(N)cc1-c1ccccn1]

4-Methyl-3-(pyridin-2-yl)aniline: To a solution of 4-methyl-3-(4,4,5,5-tetramethyl-1,3,2-dioxaborolan-2-yl)aniline (20 g, 85.80 mmol) in DMF (200 mL) and H₂O (20 mL) was added 2-bromopyridine (20.34 g, 128.70 mmol), K₂CO₃ (35.58 g, 257.38 mmol) and Pd(PPh₃)₄ (9.92 g, 8.58 mmol) at 25° C. under N₂. The mixture was stirred at 100° C. for 16 h. The reaction mixture was filtered through a celite pad, the filtrate was diluted with H₂O (200 mL) and extracted with EtOAc (3×100 mL). The combined organic layers were washed with brine (3×50 mL), dried over anhydrous Na₂SO₄, filtered and concentrated under reduced pressure. The resulting residue was adjusted to pH=3 by 1M HCl and extracted with EtOAc (3×50 mL). The organic layers were discarded, the aqueous phase was adjusted to pH=7-8 with sat. aq. NaHCO₃, then extracted with EtOAc (3×50 mL). The combined organic layers were dried over Na₂SO₄, filtered and concentrated under reduced pressure. The resulting residue was purified by silica gel column chromatography (PE:EtOAc=4:1 to 1:1) to give the titled compound. LCMS: m/z=185.0 [M+H]⁺.

### Intermediate 15

### 3-(5-Fluoropyridin-2-yl)-4-methylaniline

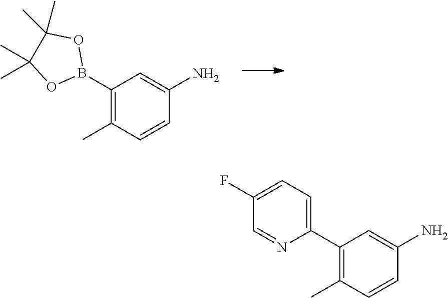 [SMILES: Cc1ccc(N)cc1B1OC(C)(C)C(C)(C)O1 ; Cc1ccc(N)cc1-c1ccc(F)cn1]

3-(5-Fluoropyridin-2-yl)-4-methylaniline: To a mixture of 4-methyl-3-(4,4,5,5-tetramethyl-1,3,2-dioxaborolan-2-yl)aniline (5 g, 21.45 mmol) and 2-bromo-5-fluoro-pyridine (5.66 g, 32.17 mmol) in DMF (50 mL) and H₂O (5 mL) was added K₂CO₃ (8.89 g, 64.35 mmol) and Pd(PPh₃)₄ (2.48 g, 2.14 mmol) at 25° C. under N₂. The mixture was stirred at 100° C. for 12 h. The reaction mixture was diluted with H₂O (100 mL) and extracted with EtOAc (3×50 mL). The combined organic layers were washed with brine (2×20 mL), dried over anhydrous Na₂SO₄, filtered and concentrated under reduced pressure. The resulting residue was purified by silica gel column chromatography (PE:MTBE=10:1 to 1:1) to give the titled compound. LCMS: m/z=203.0 [M+H]⁺.

### Intermediate 16

### 3-(Pyridin-2-yl)-4-(trifluoromethyl)aniline

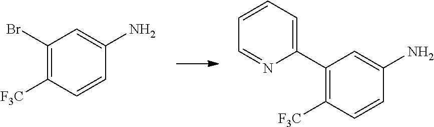 [SMILES: Nc1ccc(C(F)(F)F)c(Br)c1 ; Nc1ccc(C(F)(F)F)c(-c2ccccn2)c1]

3-(Pyridin-2-yl)-4-(trifluoromethyl)aniline: To a solution of 3-bromo-4-(trifluoromethyl)aniline (400 mg, 1.67 mmol) in DMF (5 mL) was added 2-(tributylstannyl)pyridine (736 mg, 2.00 mmol) and Pd(PPh₃)₄ (192 mg, 0.16 mmol) at 20° C. under N₂. The mixture was stirred at 110° C. for 12 h. The reaction mixture was diluted with H₂O (10 mL) and extracted with EtOAc (3×5 mL). The combined organic layers were washed with brine (10 mL), dried over anhydrous Na₂SO₄, filtered and concentrated under reduced pressure. The resulting residue was purified by silica gel column chromatography (PE:EtOAc=10:1 to 1:1) to give the titled compound. LCMS: m/z=239.1 [M+H]⁺.

### Intermediate 17

### 2-(4-Amino-2-(pyridin-2-yl)phenyl)acetonitrile

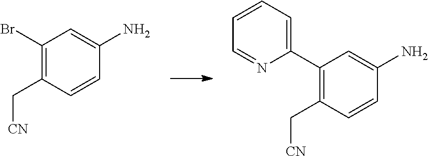 [SMILES: N#CCc1ccc(N)cc1Br ; N#CCc1ccc(N)cc1-c1ccccn1]

2-(4-Amino-2-(pyridin-2-yl)phenyl)acetonitrile: To a mixture of 2-(4-amino-2-bromophenyl)acetonitrile (210 mg, 0.99 mmol) and 2-(tributylstannyl)pyridine (440 mg, 1.19 mmol) in DMF (8 mL) was added Pd(PPh₃)₄ (115 mg, 0.10 mmol) at 20° C. under N₂. The mixture was stirred at 130° C. for 12 h. The mixture was diluted with H₂O (10 mL) and extracted with EtOAc (3×10 mL). The combined organic layers were washed with brine (10 mL), dried over anhydrous Na₂SO₄, filtered and concentrated under reduced pressure. The resulting residue was purified by silica gel column chromatography (PE:EtOAc=3:1 to 1:1) to give the titled compound. LCMS: m/z=210.1 [M+H]⁺.

### Intermediate 18

### N-(4-Ethyl-3-(5-fluoropyridin-2-yl)phenyl)-1-imidazole-1-carboxamide

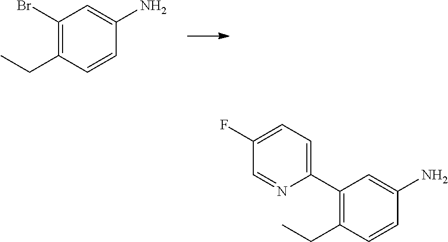

N-(4-Ethyl-3-(5-fluoropyridin-2-yl)phenyl)-1H-imidazole-1-carboxamide: To a mixture of 3-bromo-4-ethyl-aniline (200 mg, 1.00 mmol) and tributyl-(5-fluoro-2-pyridyl)stannane (463 mg, 1.20 mmol) in DMF (4 mL) was added Pd(t-Bu₃P)₂ (51 mg, 0.10 mmol) at 25° C. under N₂. The mixture was stirred at 110° C. for 12 h. The reaction mixture was diluted with H₂O (10 mL) and extracted with EtOAc (3×5 mL). The combined organic layers were washed with brine (5 mL), dried over anhydrous Na₂SO₄, filtered and concentrated under reduced pressure. The resulting residue was purified by silica gel column chromatography (PE:EtOAc=100:1 to 3:1) to give the titled compound. LCMS: m/z=217.1 [M+H]⁺.

### Intermediate 19

### 3-(3-Methoxypyridin-2-yl)-4-methylaniline

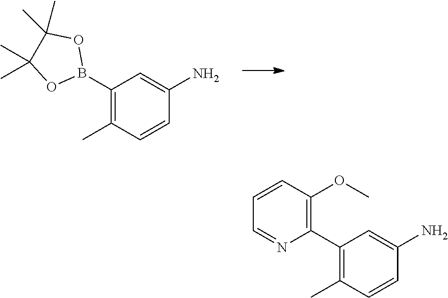 [SMILES: Cc1ccc(N)cc1B1OC(C)(C)C(C)(C)O1 ; COc1cccnc1-c1cc(N)ccc1C]

3-(3-Methoxypyridin-2-yl)-4-methylaniline: To a mixture of 4-methyl-3 (4,4,5,5-tetramethyl-1,3,2-dioxaborolan-2-yl)aniline (1 g, 4.29 mmol) and 2-chloro-3-methoxypyridine (924 mg, 6.43 mmol) in 1,4-dioxane (10 mL) and H₂O (1 mL) was added K₂CO₃ (1.78 g, 12.87 mmol) and Pd(dppf)Cl₂ (314 mg, 0.43 mmol) at 25° C. under N₂. The mixture was stirred at 100° C. for 12 h. The reaction mixture was diluted with H₂O (15 mL) and extracted with EtOAc (3×10 mL). The combined organic layers were washed with brine (5 mL), dried over anhydrous Na₂SO₄, filtered and concentrated under reduced pressure. The resulting residue was purified by silica gel column chromatography (PE:EtOAc=10:1 to 1:1) to give the titled compound. LCMS: m/z=215.1 [M+H]⁺.

### Intermediate 20

### 4-Ethyl-3-(pyridin-2-yl)aniline

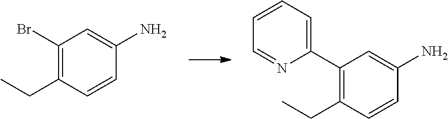

4-Ethyl-3-(pyridin-2-yl)aniline: To a mixture of 3-bromo-4-ethylaniline (200 mg, 1 mmol) and 2-(tributylstannyl)pyridine (441.61 mg, 1.20 mmol) in DMF (4 mL) was added Pd(t-Bu₃P)₂ (51.09 mg, 0.1 mmol) at 20° C. under N₂. The mixture was stirred at 110° C. for 16 h. The reaction mixture was diluted with H₂O (15 mL) and extracted with EtOAc (3×10 mL). The combined organic layers were washed with brine (3×5 mL), dried over anhydrous Na₂SO₄, filtered and concentrated under reduced pressure. The resulting residue was purified by silica gel column chromatography (PE:EtOAc=10:1 to 3:1) to give the titled compound. LCMS: m/z=199.0 [M+H]⁺.

### Intermediate 21

### 4-Amino-2-(pyridin-2-yl)benzonitrile

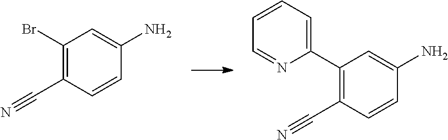 [SMILES: N#Cc1ccc(N)cc1Br ; N#Cc1ccc(N)cc1-c1ccccn1]

4-Amino-2-(pyridin-2-yl)benzonitrile: To a solution of 4-amino-2-bromo-benzonitrile (1 g, 5.08 mmol) and tributyl(2-pyridyl)stannane (2.24 g, 6.09 mmol) in DMF (20 mL) was added Pd(t-Bu₃P)₂ (259 mg, 0.51 mmol) at 25° C. under N₂. The mixture was stirred at 100° C. for 12 h and then diluted with H₂O (30 mL) and extracted with EtOAc (4×10 mL). The combined organic layers were washed with brine (30 mL), dried over anhydrous Na₂SO₄, filtered and concentrated under reduced pressure. The resulting residue was purified by silica gel column chromatography (PE:EtOAc=10:1 to 1:1) to give the titled compound. LCMS: m/z=196.2 [M+H]⁺.

### Intermediate 22

### 3-(3-Cyclopropylpyridin-2-yl)-4-methylaniline

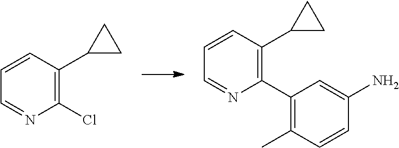 [SMILES: Clc1ncccc1C1CC1 ; Cc1ccc(N)cc1-c1ncccc1C1CC1]

3-(3-Cyclopropylpyridin-2-yl)-4-methylanilide: To a solution of 4-methyl-3-(4,4,5,5-tetramethyl-1,3,2-dioxaborolan-2-yl)aniline and 2-chloro-3-cyclopropyl-pyridine in 1,4-dioxane (10 mL) and H₂O (1 mL) was added K₂CO₃ (1.35 g, 9.77 mmol) and Pd(dppf)Cl₂ (238 mg, 3.20 mmol) under N₂. The mixture was stirred at 100° C. for 12 h. The reaction mixture was diluted with H₂O (10 mL) and extracted with EtOAc (3×10 mL). The organic layers were combined, washed with brine (10 mL), filtered and concentrated under reduced pressure. The resulting residue was purified by silica gel column chromatography (PE:EtOAc=10:1 to 1:1) to give the titled compound. LCMS: m/z=225.2 [M+H]⁺.

### Intermediate 23

### 2-(5-Amino-2-methylphenyl)nicotinonitrile

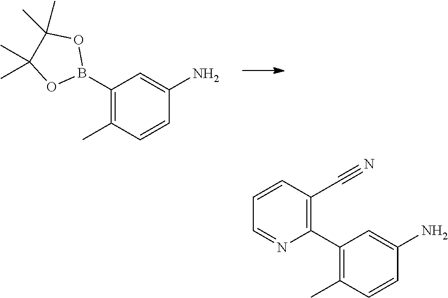 [SMILES: Cc1ccc(N)cc1B1OC(C)(C)C(C)(C)O1 ; Cc1ccc(N)cc1-c1ncccc1C#N]

2-(5-Amino-2-methylphenyl)nicotinonitrile: To a mixture of 4-methyl-3-(4,4,5,5-tetramethyl-1,3,2-dioxaborolan-2-yl)aniline (1 g, 4.29 mmol) and 2-chloronicotinonitrile (892 mg, 6.43 mmol) in 1,4-dioxane (10 mL) and H₂O (1 mL) was added K₂CO₃ (1.78 g, 12.87 mmol) and Pd(dppf)Cl₂ (314 mg, 0.43 mmol) at 25° C. under N₂. The mixture was stirred at 100° C. for 12 h. The mixture was diluted with H₂O (10 mL) and extracted with EtOAc (3×15 mL). The combined organic layers were dried over anhydrous Na₂SO₄, filtered and concentrated under reduced pressure. The resulting residue was purified by silica gel column chromatography (PE:EtOAc=3:1 to 1:1) to give the titled compound. LCMS: m/z=210.1 [M+H]⁺.

### Intermediate 24

### 4-Cyclopropyl-3-(pyridin-2-yl)aniline

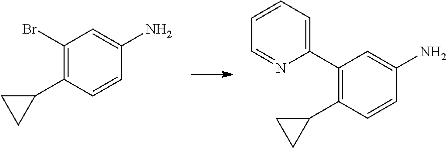 [SMILES: Nc1ccc(C2CC2)c(Br)c1 ; Nc1ccc(C2CC2)c(-c2ccccn2)c1]

4-Cyclopropyl-3-(pyridin-2-yl)aniline: To a solution of 3-bromo-4-cyclopropyl-aniline (440 mg, 2.07 mmol) in DMF (5 mL) was added tributyl(2-pyridyl)stannane (914 mg, 2.48 mmol) and Pd(t-Bu₃P)₂ (106 mg, 0.21 mmol) at 25° C. under N₂. The mixture was stirred at 110° C. for 12 h. The reaction mixture was diluted with H₂O (20 mL) and extracted with EtOAc (3×15 mL). The combined organic layers were washed with brine (15 mL), dried over anhydrous Na₂SO₄, filtered and concentrated under reduced pressure. The resulting residue was purified by silica gel column chromatography (PE:EtOAc=5:1 to 1:2) to give the titled compound. LCMS: m/z=211.1 [M+H]⁺.

### Intermediate 25

### 4-Chloro-3-(5-fluoropyridin-2-yl)aniline

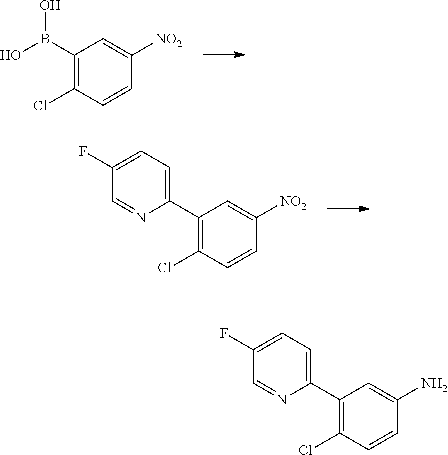 [SMILES: O=[N+]([O-])c1ccc(Cl)c(B(O)O)c1 ; O=[N+]([O-])c1ccc(Cl)c(-c2ccc(F)cn2)c1 ; Nc1ccc(Cl)c(-c2ccc(F)cn2)c1]

2-(2-Chloro-5-nitrophenyl)-5-fluoropyridine: To a solution of (2-chloro-5-nitrophenyl)boronic acid (1 g, 4.97 mmol) in DMF (15 mL) and H₂O (1.5 mL) was added 2-bromo-5-fluoropyridine (1.31 g, 7.45 mmol), K₂CO₃ (1.37 g, 9.93 mmol) and Pd(PPh₃)₄ (574 mg, 0.45 mmol) at 20° C. under N₂. The mixture was stirred at 100° C. for 12 h. The reaction mixture was diluted with H₂O (30 mL) and extracted with EtOAc (3×20 mL). The combined organic layers were washed with brine (2×10 mL), dried over anhydrous Na₂SO₄, filtered and concentrated under reduced pressure. The resulting residue was purified by silica gel column chromatography (PE:EtOAc=1:1 to 0:1) to give the titled compound. LCMS: m/z=253.0, 255.1 [M+H]⁺.

4-Chloro-3-(5-fluoropyridin-2-yl)aniline: To a solution of 2-(2-chloro-5-nitrophenyl)-5-fluoropyridine (1 g, 3.95 mmol) in EtOH (5 mL) and H₂O (1 mL) was added NH₄Cl (423 mg, 7.92 mmol) and Fe powder (1.10 g, 19.79 mmol) at 20° C. The mixture was stirred at 70° C. for 1 h. The reaction was filtered through a celite pad. The filtrate was diluted with H₂O (5 mL) and extracted with EtOAc (3×5 mL). The combined organic layers were washed with brine (5 mL), dried over anhydrous Na₂SO₄, filtered and concentrated under reduced pressure. The resulting residue was purified by silica gel column chromatography (PE:EtOAc=100:1 to 3:1) to give the titled compound. LCMS: m/z=223.1, 225.1 [M+H]⁺.

### Intermediate 26

### 4-Cyclopropyl-3-(5-fluoropyridin-2-yl)aniline

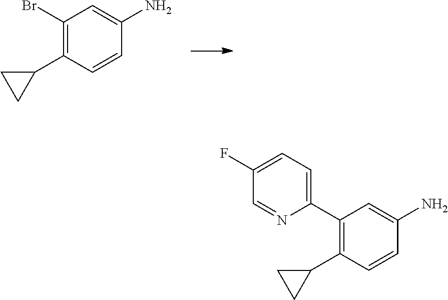 [SMILES: Nc1ccc(C2CC2)c(Br)c1 ; Nc1ccc(C2CC2)c(-c2ccc(F)cn2)c1]

4-Cyclopropyl-3-(5-fluoropyridin-2-yl)aniline: To a solution of 5-fluoro-2-(tributylstannyl)pyridine (3.06 g, 7.92 mmol) in DMF (8 mL) was added 3-bromo-4-cyclopropylaniline (1.3 g, 6.60 mmol) and Pd(t-Bu₃P)₂ (337 mg, 0.66 mmol) at 20° C. under N₂. The mixture was stirred at 100° C. for 16 h. The reaction mixture was diluted with H₂O (20 mL) and extracted with EtOAc (3×10 mL). The combined organic layers were washed with brine (2×10 mL), dried over anhydrous Na₂SO₄, filtered and concentrated under reduced pressure. The resulting residue was purified by silica gel column chromatography (PE:EtOAc=7:3 to 1:1) to give the titled compound. LCMS: m/z=229.0 [M+H]⁺.

### Intermediate 27

### N-(3-(5-Fluoropyridin-2-yl)-4-(trifluoromethyl)phenyl)-6-azabicyclo[3.1.1]heptane-6-carboxamide

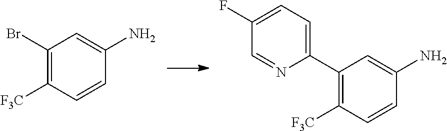 [SMILES: Nc1ccc(C(F)(F)F)c(Br)c1 ; Nc1ccc(C(F)(F)F)c(-c2ccc(F)cn2)c1]

N-(3-(5-Fluoropyridin-2-yl)-4-(trifluoromethyl)phenyl)-6-azabicyclo[3.1.1]heptane-6-carboxamide: To a solution of 3-bromo-4-(trifluoromethyl)aniline (3.00 g, 12.50 mmol) in DMF (50 mL) was added tributyl-(5-fluoro-2-pyridyl)stannane (5.79 g, 15.00 mmol), Pd(t-Bu₃P)₂ (639 mg, 1.25 mmol) at 25° C. under N₂. The mixture was stirred at 110° C. for 12 h. The reaction mixture was diluted with H₂O (100 mL) and extracted with EtOAc (3×50 mL). The combined organic layers were washed with brine (2×30 mL), dried over anhydrous Na₂SO₄, filtered and concentrated under reduced pressure. The resulting residue was purified by silica gel column chromatography (PE:EtOAc=10:1 to 3:1) to give the titled compound. LCMS: m/z=257.0 [M+H]⁺.

### Intermediate 28

### 6-(5-Amino-2-methylphenyl)picolinonitrile

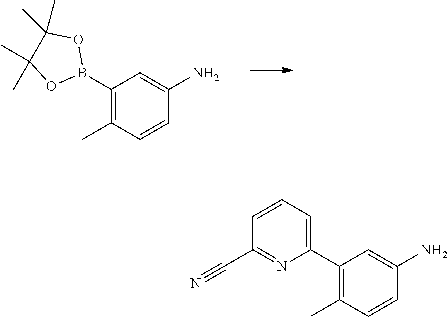 [SMILES: Cc1ccc(N)cc1B1OC(C)(C)C(C)(C)O1 ; Cc1ccc(N)cc1-c1cccc(C#N)n1]

6-(5-Amino-2-methylphenyl)picolinonitrile: To a solution of 4-methyl-3-(4,4,5,5-tetramethyl-1,3,2-dioxaborolan-2-yl)aniline (1 g, 4.29 mmol) in 1,4-dioxane (25 mL) and H₂O (2.5 mL) was added 6-bromopyridine-2-carbonitrile (0.94 g, 5.15 mmol), K₂CO₃ (1.19 g, 8.58 mmol) and Pd(dppf)Cl₂ (314 mg, 0.43 mmol) at 25° C. under N₂. The mixture was stirred at 100° C. for 4 h. The reaction mixture was filtered through a celite pad. The filtrate was concentrated under reduced pressure. The resulting residue was purified by silica gel column chromatography (PE:EtOAc=5:1 to 0:1) to give the titled compound. LCMS: m/z=210 [M+H]⁺.

### Intermediate 29

### (1S,4S)—N-(4-Methyl-3-(171yridine-2-yl)phenyl)-2,5-diazabicyclo[2.2.1]heptane-2-carboxamide hydrochloride

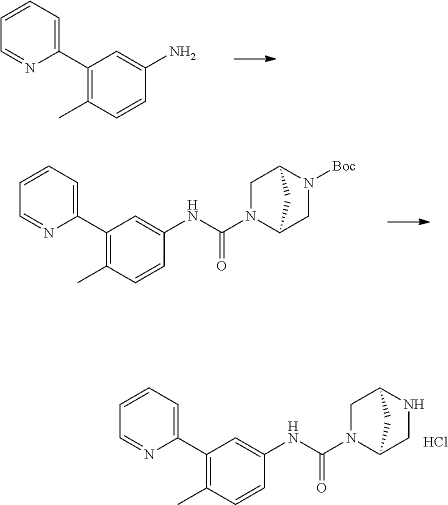 [SMILES: Cc1ccc(N)cc1-c1ccccn1 ; Cc1ccc(NC(=O)N2C[C@@H]3C[C@H]2CN3C(=O)OC(C)(C)C)cc1-c1ccccn1 ; Cc1ccc(NC(=O)N2C[C@@H]3C[C@H]2CN3)cc1-c1ccccn1 ; Cl]

(1S,4S)-tert-Butyl 5-((4-methyl-3-(pyridin-2-yl)phenyl)carbamoyl)-2,5-diazabicyclo[2.2.1]heptane-2-carboxylate: To a solution of CDI (370 mg, 2.28 mmol) in DCM (10 mL) was added dropwise a solution of 4-methyl-3-pyrimidin-2-yl-aniline (350 mg, 0.27 mmol) in DCM (10 mL) over 10 min under N₂ at −20° C. The mixture was stirred at 20° C. for 2 h. Then a mixture of tert-butyl (1S,4S)-2,5-diazabicyclo[2.2.1]heptane-2-carboxylate (250 mg, 1.26 mmol) and TEA (383 mg, 3.78 mmol) in DCM (2 mL) was added to the reaction solution. The mixture was stirred at 50° C. for an additional 0.5 h. The reaction mixture was diluted with H₂O (10 mL) and extracted with DCM (3×5 mL), the organic layers were combined, washed with brine (5 mL), dried over anhydrous Na₂SO₄, filtered and concentrated under reduced pressure. The resulting residue was purified by silica gel column chromatography (PE:EtOAc=1:2 to 0:1) to give the titled compound. LCMS: m/z=409.3 [M+H]⁺.

(1S,4S)—N-(4-Methyl-3-(pyridin-2-yl)phenyl)-2,5-diazabicyclo[2.2.1]heptane-2-carboxamide hydrochloride: To a solution of (1S,4S)-tert-butyl 5-((4-methyl-3-(pyridin-2-yl)phenyl)carbamoyl)-2,5-diazabicyclo[2.2.1]heptane-2-carboxylate (500.00 mg, 1.22 mmol) in EtOAc (5 mL) was added HCl/EtOAc (10 mL, 4 M in EtOAc), the solution was stirred at 25° C. for 1 h. The reaction mixture was concentrated under reduced pressure to give the titled compound. LCMS: m/z=309.1 [M+H]⁺.

### Intermediate 30

### (1R,4R)—N-(4-Methyl-3-(pyridin-2-yl)phenyl)-2,5-diazabicyclo[2.2.1]heptane-2-carboxamide hydrochloride

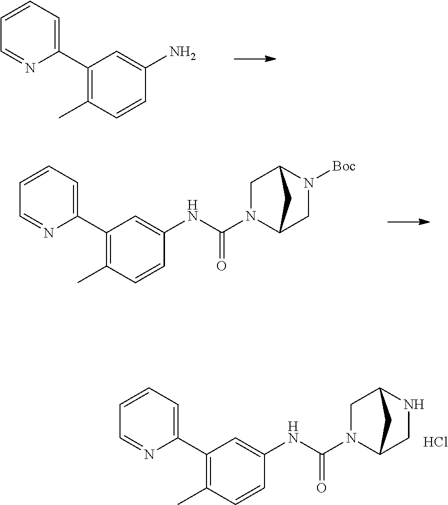 [SMILES: Cc1ccc(N)cc1-c1ccccn1 ; Cc1ccc(NC(=O)N2C[C@H]3C[C@@H]2CN3C(=O)OC(C)(C)C)cc1-c1ccccn1 ; Cc1ccc(NC(=O)N2C[C@H]3C[C@@H]2CN3)cc1-c1ccccn1 ; Cl]

(1R,4R)-tert-Butyl 5-((4-methyl-3-(pyridin-2-yl)phenyl)carbamoyl)-2,5-diazabicyclo[2.2.1]heptane-2-carboxylate: To a solution of CDI (370 mg, 2.28 mmol) in DCM (10 mL) was added dropwise a solution of 4-methyl-3-(pyridin-2-yl)aniline (350 mg, 1.9 mmol) in DCM (10 mL) over 10 min under N₂ at −20° C. The mixture was stirred at 20° C. for 2 h. Then a mixture of (1R,4R)-tert-butyl 2,5-diazabicyclo[2.2.1]heptane-2-carboxylate (214 mg, 1.08 mmol) and TEA (383 mg, 3.78 mmol) in DCM (2 mL) was added to the reaction solution. The mixture was stirred at 50° C. for an additional 2 h. The reaction mixture was diluted with H₂O (5 mL) and extracted with DCM (3×5 mL), the organic layers were combined, dried over anhydrous Na₂SO₄, filtered and concentrated under reduced pressure. The resulting residue was purified by silica gel column chromatography (PE:EtOAc=1:2 to 0:1) to give the titled compound. LCMS: m/z=409.3 [M+H]⁺.

(1R,4R)—N-(4-Methyl-3-(pyridin-2-yl)phenyl)-2,5-diazabicyclo[2.2.1]heptane-2-carboxamide hydrochloride: To a solution of (1R,4R)-tert-butyl 5-((4-methyl-3-(pyridin-2-yl)phenyl)carbamoyl)-2,5-diazabicyclo[2.2.1]heptane-2-carboxylate (500 mg, 1.22 mmol) in EtOAc (5 mL) was added HCl/EtOAc (10 mL, 4 M in EtOAc), the mixture was stirred at 25° C. for 1 h. The reaction mixture was concentrated under reduced pressure to give the titled compound. LCMS: m/z=309.1 [M+H]⁺.

### Intermediate 31

### N-[4-Methyl-3-(2-pyridyl)phenyl]-3,6-diazabicyclo[3.1.1]heptane-6-carboxamide trifluoroacetate

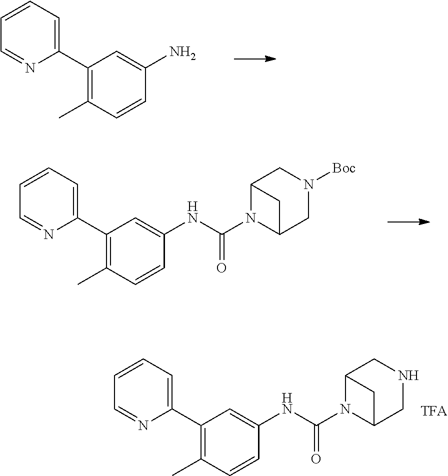 [SMILES: Cc1ccc(N)cc1-c1ccccn1 ; Cc1ccc(NC(=O)N2C3CC2CN(C(=O)OC(C)(C)C)C3)cc1-c1ccccn1 ; Cc1ccc(NC(=O)N2C3CNCC2C3)cc1-c1ccccn1 ; "TFA"]

tert-Butyl 6-((4-methyl-3-(pyridin-2-yl)phenyl)carbamoyl)-3,6-diazabicyclo[3.1.1]heptane-3-carboxylate: To a solution of CDI (2 g, 1.31 mmol) in DCM (100 mL) was added dropwise a solution of 4-methyl-3-(pyridin-2-yl)aniline (2 g, 10.86 mmol) in DCM (100 mL) over 30 min under N₂ at −20° C. The mixture was stirred at 20° C. for 2 h. Then a mixture of tert-butyl 3,6-diazabicyclo[3.1.1]heptane-3-carboxylate (1.57 g, 7.90 mmol) and TEA (2.18 g, 21.56 mmol) in DCM (10 mL) was added to the reaction solution. The mixture was stirred at 50° C. for 2 h. The reaction mixture was diluted with H₂O (20 mL) and extracted with DCM (3×10 mL). The organic layers were combined, washed with brine (20 mL), dried over anhydrous Na₂SO₄, filtered and concentrated under reduced pressure. The resulting residue was purified by silica gel column chromatography (PE:EtOAc=5:1 to 0:1) to give the titled compound. LCMS: m/z=409.2 [M+H]⁺.

N-[4-Methyl-3-(2-pyridyl)phenyl]-3,6-diazabicyclo[3.1.1]heptane-6-carboxamide trifluoroacetate: To a mixture of tert-butyl 6-((4-methyl-3-(pyridin-2-yl)phenyl)carbamoyl)-3,6-diazabicyclo[3.1.1]heptane-3-carboxylate (300 mg, 0.73 mmol) in DCM (3 mL) was added TFA (1 mL) at 25° C. The mixture was stirred at 25° C. for 12 h. The mixture was concentrated under reduced pressure to give the titled compound. LCMS: m/z=309.2 [M+H]⁺.

### Intermediate 32

### N-[4-Methyl-3-(2-pyridyl)phenyl]imidazole-1-carboxamide

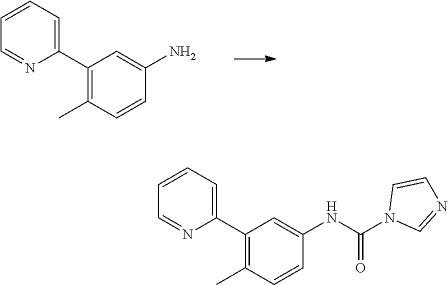 [SMILES: Cc1ccc(N)cc1-c1ccccn1 ; Cc1ccc(NC(=O)n2ccnc2)cc1-c1ccccn1]

N-[4-Methyl-3-(2-pyridyl)phenyl]imidazole-1-carboxamide To a solution of 4-methyl-3-(2-pyridyl)aniline (125.0 mg, 0.68 mmol) in DCM (6.8 mL) was added CDI (132.02 mg, 0.81 mmol). This was stirred for 12 hours at room temperature. The reaction mixture was concentrated and then to The resulting residue was added diethyl ether followed by concentration to give the titled compound. The material was used directly in the following step.

### Intermediate 33

### N-(2-Fluoro-4-methyl-5-(pyridin-2-yl)phenyl)-3,6-diazabicyclo[3.1.1]heptane-6-carboxamide trifluoroacetate

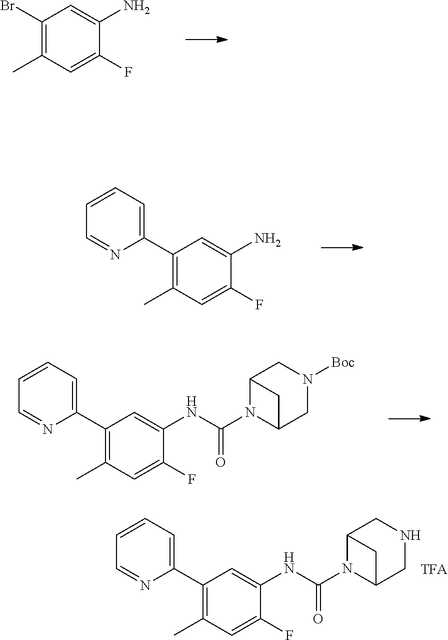

2-Fluoro-4-methyl-5-(pyridin-2-yl)aniline: To a solution of 5-bromo-2-fluoro-4-methylaniline (3 g, 14.70 mmol) in DMF (100 mL) was added 2-(tributylstannyl)pyridine (6.50 g, 17.64 mmol), Pd(PPh₃)₄ (1.70 g, 1.47 mmol) at 20° C. under N₂. The mixture was stirred at 110° C. for 12 h. The reaction was filtered through a celite pad. The filtrate was diluted with H₂O (100 mL) and extracted with EtOAc (3×100 mL). The combined organic layers were washed with brine (2×60 mL), dried over anhydrous Na₂SO₄, filtered and concentrated under reduced pressure. The resulting residue was purified by silica gel column chromatography (PE:EtOAc=20:1 to 3:1) to give the titled compound. LCMS: m/z=203.2 [M+H]⁺.

tert-Butyl 6-((2-fluoro-4-methyl-5-(pyridin-2-yl)phenyl)carbamoyl)-3,6-diazabicyclo[3.1.1]heptane-3-carboxylate: To a solution of CDI (289 mg, 1.78 mmol) in DCM (10 mL) was added dropwise a solution of 2-fluoro-4-methyl-5-(pyridin-2-yl)aniline (300 mg, 1.48 mmol) in DCM (5 mL) over 10 min under N₂ at −20° C. The mixture was stirred at 20° C. for 2 h. Then a mixture of tert-butyl 3,6-diazabicyclo[3.1.1]heptane-3-carboxylate (535 mg, 2.70 mmol) and TEA (190 mg, 1.89 mmol) in DCM (10 mL) was added to the reaction solution. The mixture was stirred at 50° C. for 2 h before being concentrated under reduced pressure. The resulting residue was purified by silica gel column chromatography (PE:EtOAc=100:1 to 1:1) to give the titled compound. LCMS: m/z=427.2 [M+H]⁺.

N-(2-Fluoro-4-methyl-5-(pyridin-2-yl)phenyl)-3,6-diazabicyclo[3.1.1]heptane-6-carboxamide trifluoroacetate: To a solution of tert-butyl 6-((2-fluoro-4-methyl-5-(pyridin-2-yl)phenyl)carbamoyl)-3,6-diazabicyclo[3.1.1]heptane-3-carboxylate (200 mg, 0.47 mmol) in DCM (4 mL) was added TFA (2.31 g, 20.26 mmol, 1.5 mL) at 25° C. The mixture was stirred at 25° C. for 2 h. The reaction was concentrated under reduced pressure to give the titled compound. LCMS: m/z=327.2 [M+H]⁺.

### Intermediate 34

### trans-3-methyl-N-(4-methyl-3-(4,4,5,5-tetramethyl-1,3,2-dioxaborolan-2-yl)phenyl)-6-azabicyclo[3.1.1]heptane-6-carboxamide

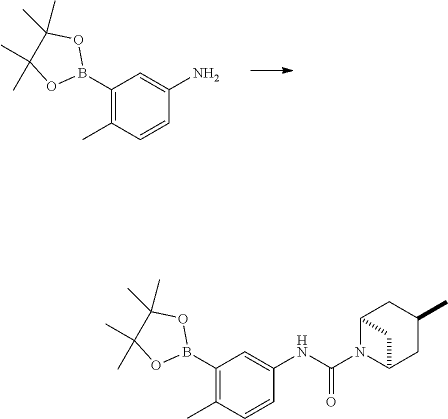

To a mixture of triphosgene (127 mg, 0.43 mmol) in THF (20 mL) was added TEA (260 mg, 2.57 mmol) and 4-methyl-3-(4,4,5,5-tetramethyl-1,3,2-dioxaborolan-2-yl)aniline (200 mg, 0.86 mmol) at 0° C. under N₂. The mixture was stirred at 20° C. for 1 h. Then trans-3-methyl-6-azabicyclo[3.1.1]heptane (129 mg, 1.16 mmol) and TEA (260 mg, 2.57 mmol) was added to the reaction solution. The reaction solution was stirred at 20° C. for 1 h. The reaction solution was diluted with H₂O (10 mL) and extracted with EtOAc (3×10 mL). The organic layers were combined, dried over anhydrous Na₂SO₄, filtered and concentrated under reduced pressure. The resulting residue was purified by silica gel column chromatography (PE:EtOAc=5:1 to 3:1) to give the titled compound. LCMS: m/z=371.2 [M+H]⁺.

### Intermediate 35

cis-3-methyl-N-(4-methyl-3-(4,4,5,5-tetramethyl-1,3,2-dioxaborolan-2-yl)phenyl)-6-azabicyclo[3.1.1]heptane-6-carboxamide

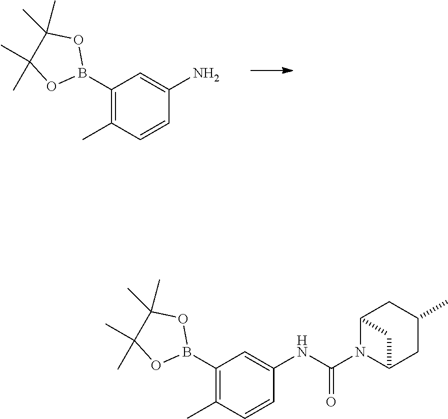

To a mixture of triphosgene (32 mg, 0.10 mmol) in THF (1.5 mL) was added TEA (65 mg, 0.64 mmol) and 4-methyl-3-(4,4,5,5-tetramethyl-1,3,2-dioxaborolan-2-yl)aniline (50 mg, 0.21 mmol) at 0° C. under N₂. The mixture was stirred at 20° C. for 1 h. Then cis-3-methyl-6-azabicyclo[3.1.1]heptane trifluoroacetate (56 mg, 0.25 mmol) and TEA (39 mg, 0.39 mmol) was added to the reaction solution. The reaction solution was stirred at 20° C. for 1 h. The reaction solution was diluted with H₂O (2 mL) and extracted with EtOAc (3×2 mL). The organic layers were combined, dried over anhydrous Na₂SO₄, filtered and concentrated under reduced pressure. The resulting residue was purified by prep-TLC (SiO₂, PE:EtOAc=1:1) to give the titled compound. LCMS: m/z=371.2 [M+H]⁺.

### Intermediate 36

### 4-methyl-3-(pyrimidin-2-yl)aniline

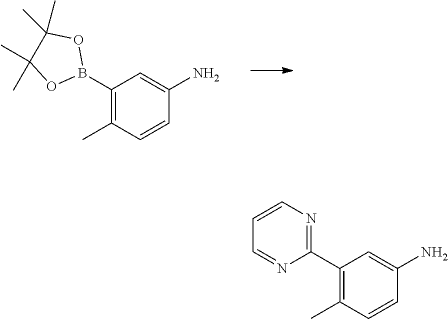 [SMILES: Cc1ccc(N)cc1B1OC(C)(C)C(C)(C)O1 ; Cc1ccc(N)cc1-c1ncccn1]

To a solution of 2-bromopyrimidine (2 g, 12.58 mmol), 4-methyl-3-(4,4,5,5-tetramethyl-1,3,2-dioxaborolan-2-yl)aniline (3.52 g, 15.10 mmol) in 1,4-dioxane (40 mL) and H₂O (4 mL) was added K₂CO₃ (5.22 g, 37.74 mmol) and Pd(dppf)Cl₂ (920 mg, 1.26 mmol) under N₂ at 25° C. The mixture was stirred at 100° C. for 12 h. The reaction mixture was diluted with H₂O (60 mL) and extracted with EtOAc (3×10 mL). The combined organic layers were washed with brine (20 mL), dried over anhydrous Na₂SO₄, filtered and concentrated under reduced pressure. The resulting residue was purified by silica gel column chromatography (PE:EtOAc=10:1 to 1:1) to give the titled compound. LCMS=186.1 [M+H]⁺.

### Intermediate 37

### 4-methyl-3-(pyrazin-2-yl)aniline

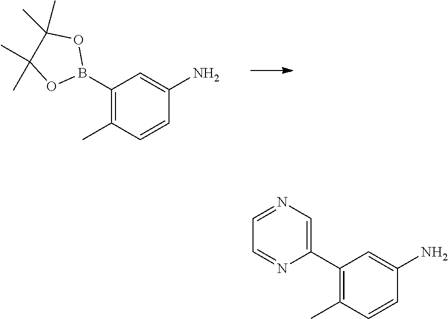 [SMILES: Cc1ccc(N)cc1B1OC(C)(C)C(C)(C)O1 ; Cc1ccc(N)cc1-c1cnccn1]

To a solution of 4-methyl-3-(4,4,5,5-tetramethyl-1,3,2-dioxaborolan-2-yl)aniline (1 g, 4.29 mmol) and 2-chloropyrazine (409 mg, 3.57 mmol) in 1,4-dioxane (20 mL) and H₂O (2 mL) was added K₂CO₃ (1.48 g, 10.72 mmol) and Pd(dppf)Cl₂ (261 mg, 0.35 mmol) under N₂ at 25° C. The mixture was stirred at 100° C. for 12 h. The reaction mixture was diluted with H₂O (30 mL) and extracted with EtOAc (3×10 mL). The combined organic layers were washed with brine (30 mL), dried over anhydrous Na₂SO₄, filtered and concentrated under reduced pressure. The resulting residue was purified by silica gel column chromatography (PE:EtOAc=10:1 to 1:1) to give the titled compound. LCMS=186.1 [M+H]⁺.

### Intermediate 38

### 4-methyl-3-(pyridazin-3-yl)aniline

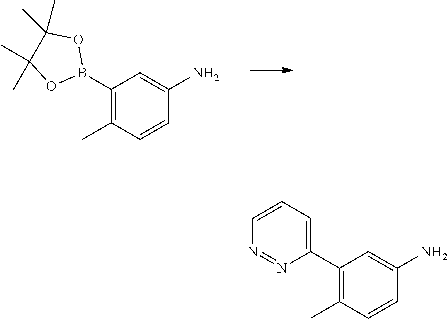 [SMILES: Cc1ccc(N)cc1B1OC(C)(C)C(C)(C)O1 ; Cc1ccc(N)cc1-c1cccnn1]

To a solution of 4-methyl-3-(4,4,5,5-tetramethyl-1,3,2-dioxaborolan-2-yl)aniline (800 mg, 3.43 mmol) in 1,4-dioxane (30 mL) and H₂O (6 mL) was added 3-bromopyridazine (818 mg, 5.15 mmol), Pd(dppf)Cl₂ (251 mg, 0.34 mmol) and K₂CO₃ (949 mg, 6.86 mmol) at 25° C. under N₂. The mixture was stirred at 105° C. for 12 h. The reaction mixture was filtered through a celite pad and the filtrate was concentrated under reduced pressure. The resulting residue was purified by silica gel column chromatography (PE:EtOAc=10:1 to 0:1) to give the titled compound. LCMS: m/z=186.0 [M+H]⁺.

### Intermediate 39

### 3-(5-fluoropyrimidin-2-yl)-4-methylaniline

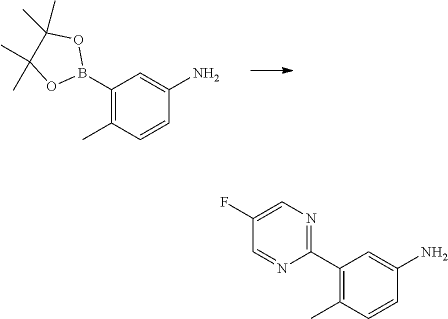 [SMILES: Cc1ccc(N)cc1B1OC(C)(C)C(C)(C)O1 ; Cc1ccc(N)cc1-c1ncc(F)cn1]

To a mixture of 4-methyl-3-(4,4,5,5-tetramethyl-1,3,2-dioxaborolan-2-yl)aniline (1 g, 4.29 mmol) and 2-bromo-5-fluoropyrimidine (1.14 g, 6.43 mmol) in DMF (20 mL) and H₂O (2 mL) was added K₂CO₃ (1.78 g, 12.87 mmol) and Pd(PPh₃)₄ (496 mg, 0.43 mmol) at 25° C. under N₂. The mixture was stirred at 100° C. for 12 h. The reaction mixture was diluted with H₂O (30 mL) and extracted with EtOAc (3×10 mL). The combined organic layers were washed with brine (3×10 mL), dried over anhydrous Na₂SO₄, filtered and concentrated under reduced pressure. The resulting residue was purified by silica gel column chromatography (PE:EtOAc=10:1 to 2:1) to give the titled compound. LCMS: m/z=204.1 [M+H]⁺.

### Intermediate 40

### 3-fluoro-4-methyl-5-(pyridin-2-yl)aniline

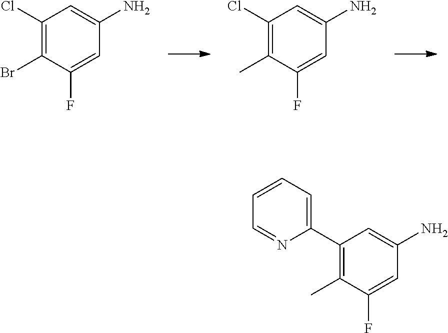

3-chloro-5-fluoro-4-methylaniline: To a mixture of 4-bromo-3-chloro-5-fluoroaniline (1 g, 4.46 mmol) and 2,4,6-trimethyl-1,3,5,2,4,6-trioxatriborinane (503 mg, 4.01 mmol) in 1,4-dioxane (20 mL) and H₂O (2 mL) was added K₂CO₃ (1.54 g, 11.14 mmol) and Pd(dppf)Cl₂ (326 mg, 0.45 mmol) at 25° C. under N₂. The mixture was stirred at 100° C. for 16 h. The mixture was diluted with H₂O (15 mL) and extracted with EtOAc (3×10 mL). The combined organic layers were washed with brine (5 mL), dried over anhydrous Na₂SO₄, filtered and concentrated under reduced pressure. The resulting residue was purified by silica gel column chromatography (PE:EtOAc=10:1 to 1:1) to give the titled compound. LCMS: m/z=160.1 [M+H]⁺.

3-fluoro-4-methyl-5-(pyridin-2-yl)aniline: To a mixture of 3-chloro-5-fluoro-4-methylaniline (300 mg, 1.88 mmol) and tributyl(2-pyridyl)stannane (1.04 g, 2.82 mmol) in DMF (5 mL) was added Pd(t-Bu₃P)₂ (96 mg, 0.19 mmol) at 25° C. under N₂. The mixture was stirred at 110° C. for 16 h. The resulting residue was diluted with H₂O (5 mL) and extracted with EtOAc (3×5 mL). The combined organic layers were washed with brine (5 mL), dried over anhydrous Na₂SO₄, filtered and concentrated under reduced pressure. The resulting residue was purified by silica gel column chromatography (PE:EtOAc=10:1 to 1:1) to give the titled compound. LCMS: m/z=203.3 [M+H]⁺.

### Intermediate 41

### cis-6-((4-methyl-3-(pyridin-2-yl)phenyl)carbamoyl)-6-azabicyclo[3.1.1]heptane-2-carboxylic acid

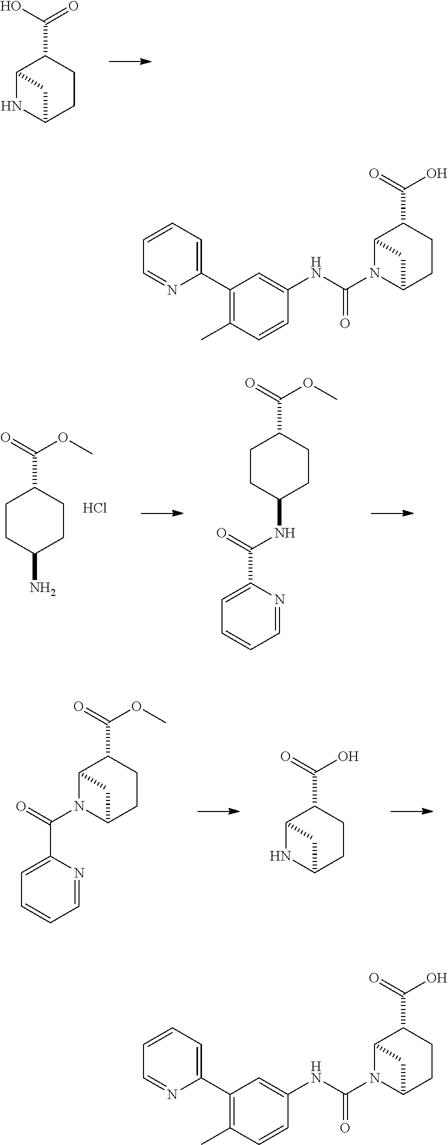

trans-methyl 4-(picolinamido)cyclohexanecarboxylate: To a solution of trans-methyl 4-aminocyclohexanecarboxylate hydrochloride (10 g, 51.63 mmol) in DCM (200 mL) was added pyridine-2-carboxylic acid (7.63 g, 61.96 mmol), EDCI (14.85 g, 77.44 mmol) and DMAP (631 mg, 5.16 mmol) at 0° C. under N2. The mixture was stirred at 20° C. for 12 h, diluted with H₂O (100 mL) and extracted with DCM (3×80 mL). The combined organic layers were dried over anhydrous Na₂SO₄, filtered and concentrated under reduced pressure. The resulting residue was purified by silica gel column chromatography (PE:EtOAc=5:1 to 0:1) to give the titled compound. LCMS: m/z=263.1 [M+H]⁺.

cis-methyl 6-picolinoyl-6-azabicyclo[3.1.1]heptane-2-carboxylate: To a solution of trans-methyl 4-(picolinamido)cyclohexanecarboxylate (3 g, 11.44 mmol) in 1,1,2,2-tetrachloroethane (90 mL) was added 1,2,3,4,5-pentafluoro-6-iodo-benzene (33.62 g, 114.37 mmol), Na₃PO4 (5.63 g, 34.31 mmol), benzoquinone (618 mg, 5.72 mmol), AgOAc (5.73 g, 34.31 mmol) and Pd(OAc)₂ (514 mg, 2.29 mmol) at 25° C. under N₂. The mixture was stirred at 140° C. for 12 h. The reaction mixture was filtered through a celite pad and the filtrate was concentrated under reduced pressure. The resulting residue was purified by silica gel column chromatography (PE:EtOAc=5:1 to 0:1) to give the titled compound. LCMS: m/z=261.1 [M+H]⁺.

cis-6-azabicyclo[3.1.1]heptane-2-carboxylic acid: To a solution of cis-methyl 6-(pyridine-2-carbonyl)-6-azabicyclo[3.1.1]heptane-2-carboxylate (1.6 g, 6.15 mmol) in EtOH (20 mL) was added NaOH (2.46 g, 61.47 mmol) at 25° C. The mixture was stirred at 90° C. for 12 h. The reaction mixture was concentrated under reduced pressure. The resulting residue was adjusted to pH=7 with 2 M HCl and the mixture was lyophilized to give the titled compound. LCMS: m/z=142.1 [M+H]⁺.

cis-6-((4-methyl-3-(pyridin-2-yl)phenyl)carbamoyl)-6-azabicyclo[3.1.1]heptane-2-carboxylic acid: To a solution of CDI (211 mg, 1.30 mmol) in DCM (5 mL) was added dropwise 4-methyl-3-(pyridin-2-yl)aniline (200 mg, 1.09 mmol) in DCM (5 mL) at −20° C. under N₂. The mixture was stirred at −20° C. for 1 h. Then cis-6-azabicyclo[3.1.1]heptane-2-carboxylic acid (152 mg, 1.08 mmol) and TEA (218 mg, 2.16 mmol) in THF (2 mL) was added. The mixture was stirred at 30° C. for 12 h and then concentrated under reduced pressure. The resulting residue was purified by prep-HPLC (column: Waters Xbridge Prep OBD C18 150×40 mm×10 m; mobile phase: A: 10 mM NH₄HCO₃ in water, B: MeCN; B % in A: 1%-30%, over 8 min) to give the titled compound. LCMS: m/z=352.2 [M+H]⁺.

### Intermediate 42

### 2,4-dimethyl-5-(pyridin-2-yl)aniline

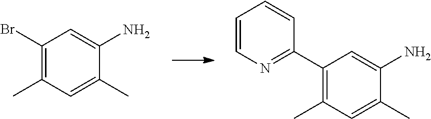

To a mixture of 5-bromo-2,4-dimethyl-aniline (300 mg, 1.50 mmol) and tributyl(2-pyridyl)stannane (717 mg, 1.95 mmol) in DMF (2 mL) was added Pd(t-Bu₃P)₂ (77 mg, 149.94 mmol) at 20° C. under N₂. The mixture was stirred at 110° C. for 12 h. The reaction mixture was diluted with H₂O (10 mL) and extracted with EtOAc (3×10 mL). The combined organic layers were dried over anhydrous Na₂SO₄, filtered and concentrated under reduced pressure. The resulting residue was purified by silica gel column chromatography (PE:EtOAc=3:1 to 1:1) to give the titled compound. LCMS: m/z=199.2 [M+H]⁺.

### Intermediate 43

### 3-chloro-4-methyl-5-(pyridin-2-yl)aniline

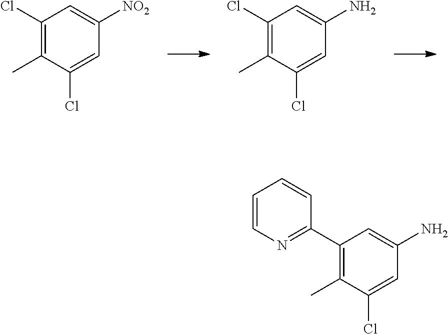

3,5-dichloro-4-methylaniline: To a solution of 1,3-dichloro-2-methyl-5-nitrobenzene (1 g, 4.85 mmol) in EtOH (10 mL) and H₂O (2 mL) was added NH₄Cl (519 mg, 9.71 mmol) and Fe (1.36 g, 24.27 mmol) at 20° C. under N₂. The mixture was stirred at 70° C. for 1 h. The reaction mixture was filtered through a celite pad. The filtrate was concentrated under reduced pressure, diluted with H₂O (10 mL) and extracted with EtOAc (3×10 mL). The combined organic layers were washed with brine (20 mL), dried over anhydrous Na₂SO₄, filtered and concentrated under reduced pressure. The resulting residue was purified by silica gel column chromatography (PE:EtOAc=100:1 to 9:1) to give the titled compound. LCMS: m/z=176.2[M+H]⁺.

3-chloro-4-methyl-5-(pyridin-2-yl)aniline: To a solution of 2-(tributylstannyl)pyridine (669 mg, 1.82 mmol) in DMF (5 mL) was added 3,5-dichloro-4-methylaniline (400 mg, 2.27 mmol) and Pd(t-Bu₃P)₂ (116 mg, 0.23 mmol) at 20° C. under N₂. The mixture was stirred at 110° C. for 12 h. The reaction mixture was filtered through a celite pad. The filtrate was diluted with H₂O (10 mL) and extracted with EtOAc (3×10 mL). The combined organic layers were washed with brine (20 mL), dried over anhydrous Na₂SO₄, filtered and concentrated under reduced pressure. The resulting residue was purified by silica gel column chromatography (PE:EtOAc=1:1 to 2:3) to give the titled compound. LCMS: m/z=219.1 [M+H]⁺.

### Intermediate 44

### 4-chloro-3-(pyridazin-3-yl)aniline

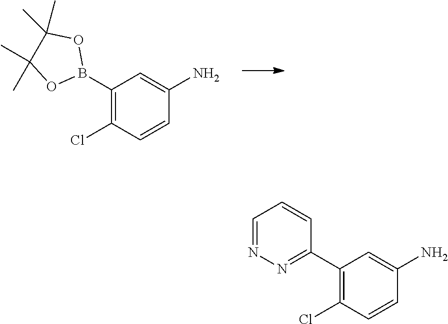 [SMILES: CC1(C)OB(c2cc(N)ccc2Cl)OC1(C)C ; Nc1ccc(Cl)c(-c2cccnn2)c1]

To a solution of 4-chloro-3-(4,4,5,5-tetramethyl-1,3,2-dioxaborolan-2-yl)aniline (200 mg, 0.78 mmol) and 3-bromopyridazine (150 mg, 0.94 mmol) in 1,4-dioxane (3 mL) was added K₃PO₄ (418 mg, 1.97 mmol) and chloro(2-dicyclohexylphosphino-2′,4′,6′-triisopropyl-1,1′-biphenyl)[2-(2′-amino-1,1′-biphenyl)palladium(II) (62 mg, 0.07 mmol) at 25° C. under N₂. The mixture was stirred at 80° C. for 12 h. The reaction mixture was diluted with H₂O (5 mL) and extracted with EtOAc (3×5 mL). The combined organic layers were washed with brine (5 mL), dried over anhydrous Na₂SO₄, filtered and concentrated under reduced pressure. The resulting residue was purified by prep-TLC (SiO₂, DCM:MeOH=10:1) to give the titled compound. LCMS=206.2 [M+H]⁺.

### Intermediate 45

### 3-bromo-4-methyl-5-(pyridin-2-yl)aniline

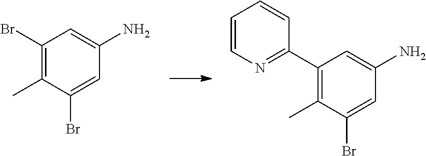

To a solution of 2-(tributylstannyl)pyridine (3.78 g, 10.27 mmol) in DMF (20 mL) was added 3,5-dibromo-4-methylaniline (3.40 g, 12.83 mmol) and Pd(PPh₃)₄ (1.48 g, 1.28 mmol) at 20° C. under N₂. The mixture was stirred at 110° C. for 12 h. The reaction mixture was filtered through a celite pad. The filtrate was diluted with H₂O (50 mL) and extracted with EtOAc (3×20 mL). The combined organic layers were washed with brine (2×20 mL), dried over anhydrous Na₂SO₄, filtered and concentrated under reduced pressure. The resulting residue was purified by silica gel column chromatography (PE:EtOAc=1:1 to 1:2) to give the titled compound. LCMS: m/z=263.1, 265.0 [M+H]⁺.

### Intermediate 46

### trans-3-ethoxy-6-azabicyclo[3.1.1]heptane trifluoroacetate

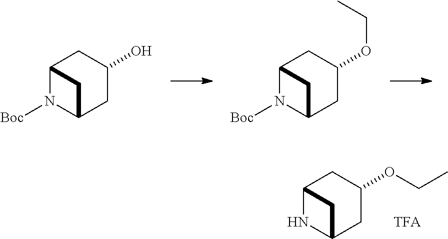

trans-tert-butyl 3-ethoxy-6-azabicyclo[3.1.1]heptane-6-carboxylate: To a solution of trans-tert-butyl 3-hydroxy-6-azabicyclo[3.1.1]heptane-6-carboxylate (50 mg, 0.23 mmol) in MeCN (2 mL) was added iodoethane (0.59 mg, 3.75 mmol) and Ag₂O (136 mg, 0.59 mmol) at 20° C. under N₂. The mixture was stirred at 80° C. for 12 h. The reaction mixture was concentrated under reduced pressure to give the crude titled compound. LCMS: m/z=186.2 [M-t-Bu+H]⁺.

trans-3-ethoxy-6-azabicyclo[3.1.1]heptane trifluoroacetate: To a solution of trans-tert-butyl 3-hydroxy-6-azabicyclo[3.1.1]heptane-6-carboxylate (50 mg, 0.21 mmol) in DCM (2 mL) was added TFA (1 mL) at 20° C. under N₂. The mixture was stirred at 20° C. for 2 h. The reaction mixture was concentrated under reduced pressure to give the titled compound. LCMS: m/z=142.2 [M+H]⁺.

### Intermediate 47

### N-(4-methyl-3-(4,4,5,5-tetramethyl-1,3,2-dioxaborolan-2-yl)phenyl)-6-azabicyclo[3.1.1]heptane-6-carboxamide

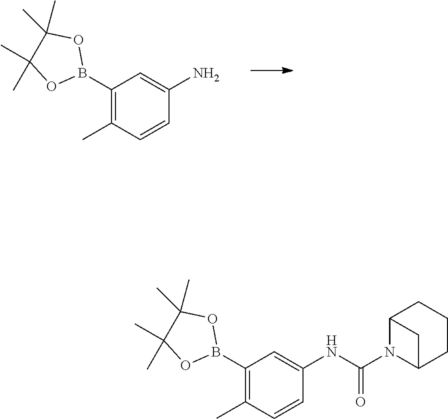 [SMILES: Cc1ccc(N)cc1B1OC(C)(C)C(C)(C)O1 ; Cc1ccc(NC(=O)N2C3CCCC2C3)cc1B1OC(C)(C)C(C)(C)O1]

To a dry round bottom flask containing triphosgene (636 mg, 2.1 mmol) was added DCM (5 mL) and the resulting solution was cooled to 0° C. A solution of 4-methyl-3-(4,4,5,5-tetramethyl-1,3,2-dioxaborolan-2-yl)aniline (500 mg, 2.1 mmol) and triethylamine (0.9 mL, 6.4 mmol) in DCM (5 mL) was added dropwise. The reaction mixture was allowed to warm from 0° C. to room temperature over 2 h followed by concentrating the reaction mixture in vacuo. The resulting residue was taken up in DCM (10.8 mL) and to that mixture was added 6-azabicyclo[3.1.1]heptane hydrochloride (315 mg, 2.4 mmol) followed by triethylamine (0.9 mL, 6.4 mmol). The reaction mixture was stirred at room temperature for 4 h. The reaction was cooled to 0° C. and quenched dropwise with sat. aq. NaHCO₃ (10 mL). The reaction was extracted with DCM (3×20 mL), dried over Na₂SO₄, filtered and concentrated in vacuo. Diethyl ether was added to the crude product and the solid was collected by filtration to afford the titled compound which was used directly in the next step. LCMS: m/z=357.2 [M+H]⁺.

### Intermediate 48

### 3-(1-methyl-1H-pyrazol-3-yl)-3,6-diazabicyclo[3.1.1]heptane trifluoroacetate

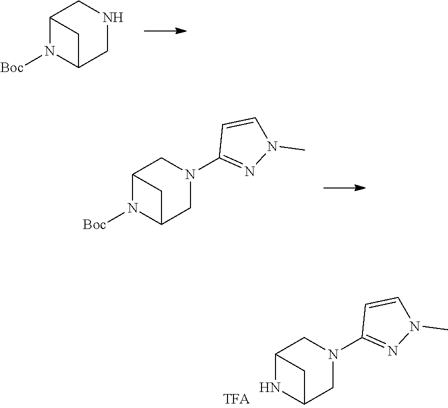 [SMILES: CC(C)(C)OC(=O)N1C2CNCC1C2 ; Cn1ccc(N2CC3CC(C2)N3C(=O)OC(C)(C)C)n1 ; Cn1ccc(N2CC3CC(C2)N3)n1]

tert-butyl 3-(1-methyl-1H-pyrazol-3-yl)-3,6-diazabicyclo[3.1.1]heptane-6-carboxylate: To a solution of 3-iodo-1-methyl-1H-pyrazole (2.31 g, 11.10 mmol) in toluene (15 mL) was added tert-butyl 3,6-diazabicyclo[3.1.1]heptane-6-carboxylate (2.00 g, 10.09 mmol), t-BuONa (1.94 g, 20.18 mmol), Xphos (962 mg, 2.02 mmol) and Pd₂(dba)₃ (924 mg, 1.01 mmol) at 20° C. under N₂. The mixture was stirred at 110° C. for 12 h, filtered through a celite pad and the filtrate was concentrated under reduced pressure. The resulting residue was purified by silica gel column chromatography (PE:EtOAc=1:2 to 1:4) to give the titled compound. LCMS: m/z=279.2 [M+H]⁺.

3-(1-methyl-1H-pyrazol-3-yl)-3,6-diazabicyclo[3.1.1]heptane trifluoroacetate: To a solution of tert-butyl 3-(1-methyl-1H-pyrazol-3-yl)-3,6-diazabicyclo[3.1.1]heptane-6-carboxylate (300 mg, 1.08 mmol) in DCM (5 mL) was added TFA (5 mL) at 20° C. under N₂. The mixture was stirred at 20° C. for 2 h. The reaction mixture was concentrated under reduced pressure to give the titled compound. LCMS: m/z=179.2 [M+H]⁺.

### Intermediate 49

### 2-(5-amino-2-methyl-phenyl)-5-fluoro-pyrimidine-4-carbonitrile

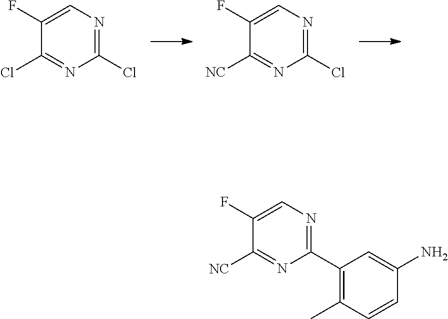 [SMILES: Fc1cnc(Cl)nc1Cl ; N#Cc1nc(Cl)ncc1F ; Cc1ccc(N)cc1-c1ncc(F)c(C#N)n1]

2-chloro-5-fluoropyrimidine-4-carbonitrile: To a solution of 2,4-dichloro-5-fluoro-pyrimidine (5 g, 29.95 mmol) and DABCO (336 mg, 2.99 mmol) in DMF (50 mL) and H₂O (10 mL) was added NaCN (1.61 g, 32.94 mmol) at 0° C. The mixture was stirred at 0° C. for 1 h. The reaction mixture was diluted with H₂O (10 mL) and extracted with EtOAc (3×10 mL). The combined organic layers were washed with brine (3×10 mL), dried over anhydrous Na₂SO₄, filtered and concentrated under reduced pressure. The residue was purified by silica gel chromatography (PE:EtOAc=1:0 to 0:1) to give the titled compound.

2-(5-amino-2-methylphenyl)-5-fluoropyrimidine-4-carbonitrile: To a mixture of 4-methyl-3-(4,4,5,5-tetramethyl-1,3,2-dioxaborolan-2-yl)aniline (500 mg, 2.14 mmol) in 1,4-dioxane (5 mL) and H₂O (0.5 mL) at 20° C. under N₂ was added 2-chloro-5-fluoro-pyrimidine-4-carbonitrile (675 mg, 4.29 mmol), Pd(dppf)Cl₂ (156 mg, 0.22 mmol) and K₂CO₃ (889 mg, 6.43 mmol). The reaction was heated to 100° C. and stirred for 2 h. The reaction was diluted with H₂O (5 mL) and extracted with EtOAc (3×5 mL). The combined organic layers were washed with brine (5 mL), dried over anhydrous Na₂SO₄, filtered and concentrated under reduced pressure. The resulting residue was purified by silica gel chromatography (PE:EtOAc=3:1 to 0:1) and further purified by prep-TLC (SiO₂, PE:EtOAc=1:1) to give the titled compound. LCMS: m/z=229.2 [M+H]⁺.

### Intermediate 50

### 2,3-difluoro-5-(5-fluoropyrimidin-2-yl)-4-methylaniline

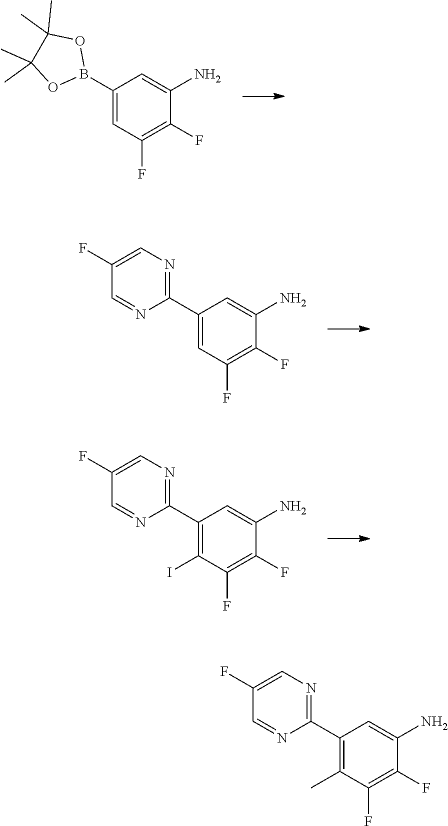 [SMILES: CC1(C)OB(c2cc(N)c(F)c(F)c2)OC1(C)C ; Nc1cc(-c2ncc(F)cn2)cc(F)c1F ; Nc1cc(-c2ncc(F)cn2)c(I)c(F)c1F ; Cc1c(-c2ncc(F)cn2)cc(N)c(F)c1F]

2,3-difluoro-5-(5-fluoropyrimidin-2-yl)aniline: To a solution of 2,3-difluoro-5-(4,4,5,5-tetramethyl-1,3,2-dioxaborolan-2-yl)aniline (500 mg, 1.96 mmol) in 1,4-dioxane (10 mL) and H₂O (1 mL) was added 2-bromo-5-fluoro-pyrimidine (416 mg, 2.35 mmol) K₂CO₃ (541 mg, 3.92 mmol) and Pd(dppf)Cl₂ (143 mg, 0.20 mmol) at 20° C. under N₂. The mixture was stirred at 100° C. for 5 h. The reaction mixture was filtered through a celite pad and the filtrate was concentrated under reduced pressure. The residue was purified by silica gel column chromatography (PE:EtOAc=10:1 to 1:1) to give the titled compound. LCMS: m/z=226.1 [M+H]⁺.

2,3-difluoro-5-(5-fluoropyrimidin-2-yl)-4-iodoaniline: To a solution of 2,3-difluoro-5-(5-fluoropyrimidin-2-yl)aniline (270 mg, 1.20 mmol) in MeCN (10 mL) at 0° C. under N₂. was added NIS (270 mg, 1.2 mmol). The mixture was warmed to 20° C. and stirred for 12 h. The reaction mixture was diluted with H₂O (10 mL) and extracted with EtOAc (3×15 mL). The combined organic layers were washed with brine (10 mL), dried over anhydrous Na₂SO₄, filtered and concentrated under reduced pressure. The resulting residue was purified by silica gel column chromatography (PE:EtOAc=10:1 to 1:1) to give the titled compound. LCMS: m/z=352.0 [M+H]⁺.

2,3-difluoro-5-(5-fluoropyrimidin-2-yl)-4-methylaniline: To a solution of 2,3-difluoro-5-(5-fluoropyrimidin-2-yl)-4-iodo-aniline (120 mg, 0.34 mmol) in 1,4-dioxane (2 mL) and H₂O (0.2 mL) was added 2,4,6-trimethyl-1,3,5,2,4,6-trioxatriborinane (172 mg, 0.68 mmol, 50% purity), K₂CO₃ (142 mg, 1.03 mmol) and Pd(dppf)Cl₂ (251 mg, 0.34 mmol) at 20° C. under N₂. The mixture was heated at 100° C. and stirred for 3 h. The reaction mixture was filtered through a celite pad and the filtrate was concentrated under reduced pressure. The residue was purified by prep-TLC (SiO₂, PE:EtOAc=5:1) to give the titled compound. LCMS: m/z=240.2 [M+H]⁺.

### Intermediate 51

### 4-(((tert-butyldimethylsilyl)oxy)methyl)-3-(5-fluoropyrimidin-2-yl)aniline

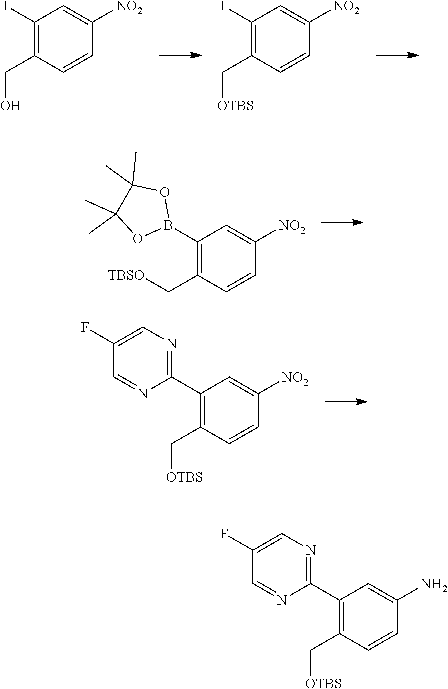

tert-butyl((2-iodo-4-nitrobenzyl)oxy)dimethylsilane: To a solution of (2-iodo-4-nitro-phenyl)methanol (1 g, 3.58 mmol) and TBSCl (648 mg, 4.30 mmol) in DCM (10 mL) was added imidazole (293 mg, 4.30 mmol). The mixture was stirred at 20° C. for 1 h. The reaction mixture was diluted with H₂O (10 mL) and extracted with DCM (3×10 mL). The combined organic layers were washed with brine (10 mL), dried over anhydrous Na₂SO₄, filtered and concentrated under reduced pressure. The resulting residue was purified by silica gel column chromatography (PE:EtOAc=10:1 to 3:1) to give the titled compound.

tert-butyldimethyl((4-nitro-2-(4,4,5,5-tetramethyl-1,3,2-dioxaborolan-2-yl)benzyl)oxy)silane: To a solution of tert-butyl((2-iodo-4-nitrobenzyl)oxy)dimethylsilane (1 g, 2.54 mmol) and 4,4,5,5-tetramethyl-2-(4,4,5,5-tetramethyl-1,3,2-dioxaborolan-2-yl)-1,3,2-dioxaborolane (1.29 g, 5.09 mmol) in 1,4-dioxane (10 mL) was added KOAc (749 mg, 7.63 mmol) and Pd(dppf)Cl₂CH₂Cl₂ (208 mg, 0.25 mmol) at 20° C. under N₂. The mixture was heated to 100° C. and stirred for 3 h. The reaction mixture was filtered and concentrated under reduced pressure. The resulting residue was purified by silica gel column chromatography (PE:EtOAc=100:1 to 10:1) to give the titled compound. LCMS: m/z=394.2 [M+H]⁺.

2-(2-(((tert-butyldimethylsilyl)oxy)methyl)-5-nitrophenyl)-5-fluoropyrimidine: To a mixture of tert-butyldimethyl((4-nitro-2-(4,4,5,5-tetramethyl-1,3,2-dioxaborolan-2-yl)benzyl)oxy)silane (400 mg, 1.02 mmol) and 2-bromo-5-fluoro-pyrimidine (216 mg, 1.22 mmol) in 1,4-dioxane (5 mL) and H₂O (0.5 mL) was added Pd(dppf)Cl₂ (74 mg, 0.10 mmol) and K₂CO₃ (281 mg, 2.03 mmol) at 20° C. under N₂. The mixture was heated to 100° C. and stirred for 3 h. The reaction mixture was diluted with H₂O (10 mL) and extracted with EtOAc (3×10 mL). The combined organic layers were washed with brine (10 mL), dried over anhydrous Na₂SO₄, filtered and concentrated under reduced pressure. The resulting residue was purified by silica gel column chromatography (PE:EtOAc=10:1 to 5:1) to give the titled compound. LCMS: m/z=364.1 [M+H]⁺.

4-(((tert-butyldimethylsilyl)oxy)methyl)-3-(5-fluoropyrimidin-2-yl)aniline: To a solution of 2-(2-(((tert-butyldimethylsilyl)oxy)methyl)-5-nitrophenyl)-5-fluoropyrimidine (1.5 g, 4.13 mmol) in EtOH (15 mL) and H₂O (15 mL) was added NH₄Cl (1.10 g, 20.64 mmol) and Fe (1.15 g, 20.64 mmol) at 20° C. The mixture was then heated at 90° C. and stirred for 1 h. The reaction mixture was diluted with H₂O (20 mL) and extracted with EtOAc (3×10 mL). The combined organic layers were washed with brine (10 mL), dried over anhydrous Na₂SO₄, filtered and concentrated under reduced pressure. The residue was purified by silica gel column chromatography (PE:EtOAc=10:1 to 3:1) to give the titled compound. LCMS: m/z=334.1 [M+H]⁺.

### Intermediate 53

### 2-fluoro-4-methyl-5-(1,3,5-triazin-2-yl)aniline

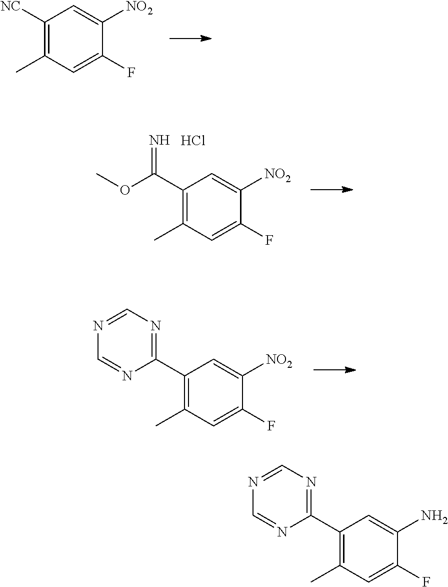

Methyl 4-fluoro-2-methyl-5-nitrobenzimidate hydrochloride: A solution of 4-fluoro-2-methyl-5-nitro-benzonitrile (1 g, 5.55 mmol) in HCl/MeOH (15 mL) was stirred at 20° C. for 16 h. The mixture was concentrated under reduced pressure. The residue was slurried with MTBE (10 mL), filtered and the filter cake was dried under reduced pressure to give the titled compound. LCMS: m/z=213.1 [M+H]⁺.

2-(4-fluoro-2-methyl-5-nitrophenyl)-1,3,5-triazine: To a solution of methyl 4-fluoro-2-methyl-5-nitrobenzimidate hydrochloride (500 mg, 2.36 mmol) in EtOH (10 mL) was added 1,3,5-triazine (210 mg, 2.59 mmol) and AcOH (283 mg, 4.71 mmol) at 20° C. under N₂. The mixture was heated to 80° C. and stirred for 16 h. The mixture was concentrated under reduced pressure and the residue was purified by silica gel column chromatography (PE:EtOAc=5:1) to give the titled compound. LCMS: m/z=235.1 [M+H]⁺.

2-fluoro-4-methyl-5-(1,3,5-triazin-2-yl)aniline: To a solution of 2-(4-fluoro-2-methyl-5-nitrophenyl)-1,3,5-triazine (100 mg, 0.43 mmol) in EtOH (4 mL) and H₂O (1 mL) was added Fe (119 mg, 2.14 mmol) and NH₄Cl (114 mg, 2.14 mmol) at 20° C. The mixture was heated to 80° C. and stirred for 2 h. The reaction mixture was concentrated under reduced pressure and the crude product was slurried with DCM:MeOH (V₁:V₂=10:1). Then the filtrate was concentrated under reduced pressure to give the titled compound. LCMS: m/z=205.2 [M+H]⁺.

### Intermediate 54

### 1-(3-(2-aminoethoxy)pyridin-2-yl)ethanone

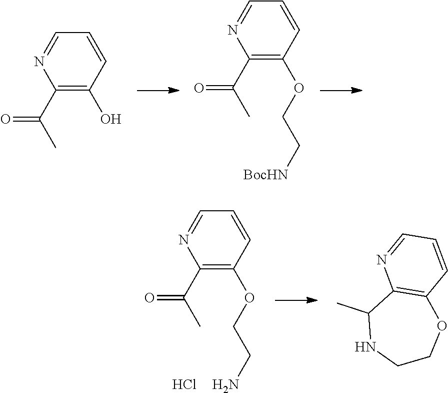

tert-butyl (2-((2-acetylpyridin-3-yl)oxy)ethyl)carbamate: To a solution of 1-(3-hydroxypyridin-2-yl)ethanone (2 g, 14.58 mmol) in DMF (25 mL) was added K₂CO₃ (4.03 g, 29.17 mmol) at 25° C. under N₂. The mixture was stirred at 25° C. for 0.5 h and then tert-butyl (2-bromoethyl)carbamate (3.6 g, 16.04 mmol) was added to the solution at 25° C. The mixture was heated to 70° C. and stirred for 12.5 h. Then the reaction mixture was diluted with H₂O (75 mL) and extracted with EtOAc (3×50 mL). The combined organic layers were washed with brine (75 mL), dried over anhydrous Na₂SO₄, filtered and concentrated under reduced pressure. The resulting residue was purified by silica gel column chromatography (PE:EtOAc=3:1 to 1:2) to give the titled compound. LCMS: m/z=281.2 [M+H]⁺.

1-(3-(2-aminoethoxy)pyridin-2-yl)ethanone hydrochloride: A solution of tert-butyl (2-((2-acetylpyridin-3-yl)oxy)ethyl)carbamate (3.2 g, 11.42 mmol) in HCl/EtOAc (4 M, 90 mL) was stirred at 20° C. for 2 h. The reaction mixture was concentrated under reduced pressure to give the titled compound.

LCMS: m/z=181.1 [M+H]⁺. [0402]5-methyl-2,3,4,5-tetrahydropyrido[2,3-][1,4]oxazepine: To a solution of 1-(3-(2-aminoethoxy)-2-pyridyl)ethanone hydrochloride (1 g, 4.62 mmol) in MeOH (15 mL) was added TEA (934 mg, 9.23 mmol) and AcOH (554 mg, 9.23 mmol) at 25° C. under N₂ and stirred for 2 h. Then the reaction was cooled to 0° C. and NaBH₃CN (580 mg, 9.23 mmol) was added under N₂. The mixture was warmed to 25° C. and stirred for 1 h. The reaction mixture was adjusted to pH=7 with aq. sat. Na₂CO₃ and extracted with EtOAc (3×15 mL). The combined organic layers were washed with brine (20 mL), dried over anhydrous Na₂SO₄, filtered and concentrated under reduced pressure to give the titled compound. The resulting crude material was used directly in the next step. LCMS: m/z=165.2 [M+H]⁺.

### Intermediate 55

### (2S,4S)-1-(tert-butoxycarbonyl)-4-(trifluoromethyl)pyrrolidine-2-carboxylic acid

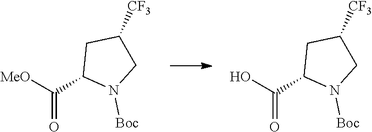 [SMILES: COC(=O)[C@@H]1C[C@H](C(F)(F)F)CN1C(=O)OC(C)(C)C ; CC(C)(C)OC(=O)N1C[C@@H](C(F)(F)F)C[C@H]1C(=O)O]

(2S,4S)-1-(tert-butoxycarbonyl)-4-(trifluoromethyl)pyrrolidine-2-carboxylic acid: To a solution of 1-(tert-butyl) 2-methyl (2S,4S)-4-(trifluoromethyl)pyrrolidine-1,2-dicarboxylate (410 mg, 1.38 mmol) in THF (4 mL) and H₂O (1 mL) was added LiOH (132 mg, 5.52 mmol) at 25° C. under N₂ and the mixture was stirred for 6 h. The reaction mixture was diluted with H₂O (3 mL), adjusted to pH ˜5-6 with 2 N HCl and extracted with EtOAc (3×5 mL). The combined organic layers were washed with brine (5 mL), dried over anhydrous Na₂SO₄, filtered and concentrated under reduced pressure. The resulting residue was purified by silica gel column chromatography (PE:EtOAc=10:1 to 1:1) to give the titled compound.

### Intermediate 56

### trans-1-(methoxymethyl)-3-methyl-6-azabicyclo[3.1.1]heptane

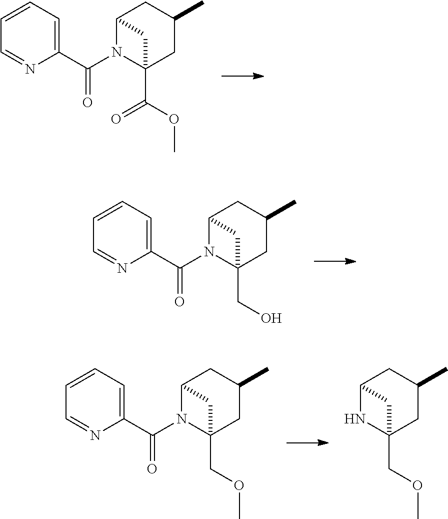

(trans-1-(hydroxymethyl)-3-methyl-6-azabicyclo[3.1.1]heptan-6-yl)(pyridin-2-yl)methanone: To a solution of methyl trans-3-methyl-6-picolinoyl-6-azabicyclo[3.1.1]heptane-1-carboxylate (1 g, 3.65 mmol) and CaCl₂ (1.62 g, 14.58 mmol) in MeOH (30 mL) at 0° C. under N₂ was added NaBH₄ (828 mg, 21.87 mmol). The reaction was then warmed to 20° C. and stirred for 1 h. The reaction was quenched with aq. sat. NH₄Cl (20 mL) and extracted with EtOAc (3×15 mL). The combined organic layers were washed with brine (3×5 mL), dried over anhydrous Na₂SO₄, filtered and concentrated under reduced pressure. The resulting residue was purified by silica gel column chromatography (PE:EtOAc=3:1 to 1:1) to give the titled compound. LCMS: m/z=247.2 [M+H]⁺.

(trans-1-(methoxymethyl)-3-methyl-6-azabicyclo[3.1.1]heptan-6-yl)(pyridin-2-yl)methanone: To a solution of (trans-1-(hydroxymethyl)-3-methyl-6-azabicyclo[3.1.1]heptan-6-yl)(pyridin-2-yl)methanone (600 mg, 2.44 mmol) in DMF (12 mL) at 0° C. was added NaH (146 mg, 3.65 mmol, 60% in mineral oil). The reaction mixture was warmed to 20° C. and stirred for 0.5 h. Then the reaction was cooled to 0° C. and MeI (692 mg, 4.87 mmol) was added. The mixture was warmed up to 20° C. and stirred for 2 h. The reaction was quenched by aq. sat. NH₄Cl (5 mL) and extracted with EtOAc (3×5 mL). The combined organic layers were washed with brine (3×2 mL), dried over anhydrous Na₂SO₄, filtered and concentrated under reduced pressure. The resulting residue was purified by silica gel column chromatography (PE:EtOAc=5:1 to 1:1) to give the titled compound. LCMS: m/z=261.2 [M+H]⁺.

trans-1-(methoxymethyl)-3-methyl-6-azabicyclo[3.1.1]heptane: To a solution of (trans-1-(methoxymethyl)-3-methyl-6-azabicyclo[3.1.1]heptan-6-yl)(pyridin-2-yl)methanone (450 mg, 1.73 mmol) in EtOH (10 mL) was added NaOH (692 mg, 17.29 mmol) at 25° C. under N₂. The mixture was heated to 90° C. and stirred for 16 h. Then the reaction mixture was concentrated under reduced pressure (water pump, below 35° C.) to give the crude product. The crude material was stirred in DCM (20 mL), filtered through a celite pad, and concentrated under reduce pressure (water pump, below 35° C.). If the residue still contains solids, the above procedure was repeated until no solid remained (2-3 times) to give the titled compound. LCMS: m/z=156.2 [M+H]⁺.

### Intermediates 57 and 58

### (cis-1-(1-methoxyethyl)-3-methyl-6-azabicyclo[3.1.1]heptan-6-yl)(pyridin-2-yl)methanone and (cis-1-(1-methoxyethyl)-3-methyl-6-azabicyclo[3.1.1]heptan-6-yl)(pyridin-2-yl)methanone

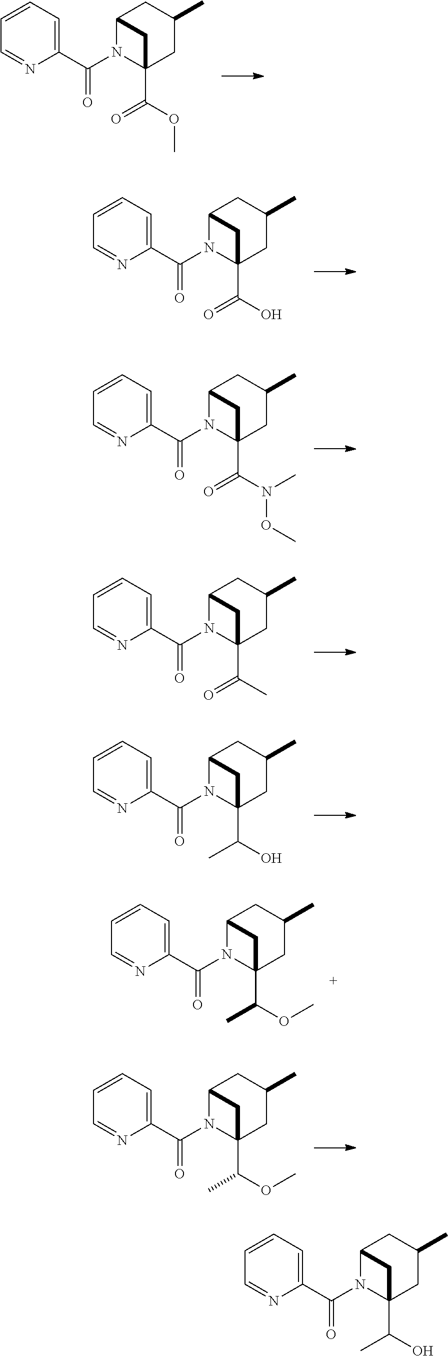

cis-3-methyl-6-picolinoyl-6-azabicyclo[3.1.1]heptane-1-carboxylic acid: To a solution of methyl cis-3-methyl-6-picolinoyl-6-azabicyclo[3.1.1]heptane-1-carboxylate (2 g, 7.29 mmol) in MeOH (20 mL) at 20° C. was added a solution of NaOH (437 mg, 10.94 mmol) in H₂O (4 mL) and the reaction mixture was stirred for 3 h. The reaction mixture was adjusted to pH=3 by addition of 2 M HCl. The resulting mixture was extracted with EtOAc (3×10 mL). The combined organic layers were washed with brine (10 mL), dried over anhydrous Na₂SO₄, filtered and concentrated under reduced pressure to give the titled compound. LCMS: m/z=261.2 [M+H]⁺.

cis-N-methoxy-N,3-dimethyl-6-picolinoyl-6-azabicyclo[3.1.1]heptane-1-carboxamide: To a solution of cis-3-methyl-6-picolinoyl-6-azabicyclo[3.1.1]heptane-1-carboxylic acid (1.7 g, 6.53 mmol) in DMF (20 mL) was added N,O-dimethylhydroxylamine hydrochloride (701 mg, 7.18 mmol), HATU (2.73 g, 7.18 mmol) and DIEA (2.53 g, 19.59 mmol) at 20° C. under N₂ and the mixture was stirred for 3 h. The mixture was diluted with H₂O (50 mL) and extracted with EtOAc (3×20 mL). The organic layers were combined, washed with brine (3×10 mL), dried over anhydrous Na₂SO₄, filtered and concentrated under reduced pressure. The resulting residue was purified by silica gel column chromatography (PE:EtOAc=10:1 to 3:1) to give the titled compound. LCMS: m/z=304.2 [M+H]⁺.

1-(cis-3-methyl-6-picolinoyl-6-azabicyclo[3.1.1]heptan-1-yl)ethan-1-one: To a solution of cis-N-methoxy-N,3-dimethyl-6-(pyridine-2-carbonyl)-6-azabicyclo[3.1.1]heptane-1-carboxamide (1.7 g, 5.60 mmol) in THF (20 mL) at −78° C. under N₂ was added MeLi (2.7 M in THF, 2.70 mL). The reaction mixture was stirred at −78° C. for 2 h. The mixture was quenched by addition of aq. sat. NH₄Cl (20 mL) and extracted with EtOAc (3×10 mL). The combined organic layers were washed with brine (10 mL) dried over anhydrous Na₂SO₄, filtered and concentrated under reduced pressure. The resulting residue was purified by silica gel column chromatography (PE:EtOAc=5:1 to 1:1) to give the titled compound. LCMS: m/z=259.2 [M+H]⁺.

(cis-1-(1-hydroxyethyl)-3-methyl-6-azabicyclo[3.1.1]heptan-6-yl)(pyridin-2-yl)methanone and (cis-1-(1-hydroxyethyl)-3-methyl-6-azabicyclo[3.1.1]heptan-6-yl)(pyridin-2-yl)methanone: To a solution of 1-(cis-3-methyl-6-picolinoyl-6-azabicyclo[3.1.1]heptan-1-yl)ethan-1-one (750 mg, 2.90 mmol) in MeOH (10 mL) at 0° C. was added NaBH₄ (220 mg, 5.81 mmol) and the mixture was stirred for 2 h. The mixture was quenched by addition of aq. sat. NH₄Cl (10 mL) at 0° C. The reaction mixture was concentrated under reduced pressure to remove the organics and the aqueous was extracted with EtOAc (3×10 mL). The combined organic layers were washed with brine (10 mL), dried over anhydrous Na₂SO₄, filtered and concentrated under reduced pressure to give the titled compounds as a mixture of 4 diastereomers. LCMS: m/z=261.2 [M+H]⁺.

(cis-1-(1-methoxyethyl)-3-methyl-6-azabicyclo[3.1.1]heptan-6-yl)(pyridin-2-yl)methanone and (cis-1-(1-methoxyethyl)-3-methyl-6-azabicyclo[3.1.1]heptan-6-yl)(pyridin-2-yl)methanone: To a solution of (cis-1-(1-hydroxyethyl)-3-methyl-6-azabicyclo[3.1.1]heptan-6-yl)(pyridin-2-yl)methanone and (cis-1-(1-hydroxyethyl)-3-methyl-6-azabicyclo[3.1.1]heptan-6-yl)(pyridin-2-yl)methanone (700 mg, 2.69 mmol) in DMF (8 mL) at 0° C. under N₂ was added NaH (161 mg, 4.03 mmol, 60% in mineral oil). The mixture was stirred to 0° C. for 0.5 h and then MeI (763 mg, 5.38 mmol) was added and stirred an additional 2 h. The mixture was quenched by addition of aq. sat. NH₄Cl (30 mL) and extracted with EtOAc (3×10 mL). The combined organic layers were washed with brine (10 mL), dried over anhydrous Na₂SO₄, filtered and concentrated under reduced pressure. The resulting residue was purified by silica gel column chromatography (PE:EtOAc=3:1 to 1:1) to give (cis-1-(1-methoxyethyl)-3-methyl-6-azabicyclo[3.1.1]heptan-6-yl)(pyridine-2-yl)methanone, Intermediate 57, as the first eluting mixture of enantiomers and Intermediate 58 as a mixture of (cis-1-(1-methoxyethyl)-3-methyl-6-azabicyclo[3.1.1]heptan-6-yl)(pyridinedin-2-yl)methanone as the second eluting mixture of enantiomers and unreacted (cis-1-(1-hydroxyethyl)-3-methyl-6-azabicyclo[3.1.1]heptan-6-yl)(pyridinedin-2-yl)methanone LCMS: m/z=275.1 [M+H]⁺.

### Intermediate 59

### cis-1-(1-methoxyethyl)-3-methyl-6-azabicyclo[3.1.1]heptane and 1-(cis-3-methyl-6-azabicyclo[3.1.1]heptan-1-yl)ethan-1-ol

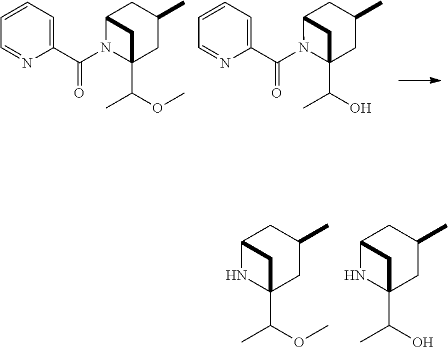

cis-1-(1-methoxyethyl)-3-methyl-6-azabicyclo[3.1.1]heptane and 1-(cis-3-methyl-6-azabicyclo[3.1.1]heptan-1-yl)ethan-1-ol: To Intermediate 58, a mixture of (cis-1-(1-methoxyethyl)-3-methyl-6-azabicyclo[3.1.1]heptan-6-yl)(pyridinedin-2-yl)methanone and (cis-1-(1-hydroxyethyl)-3-methyl-6-azabicyclo[3.1.1]heptan-6-yl)(pyridinedin-2-yl)methanone (400 mg, 1.46 mmol), in THF (10 mL) at 0° C. under N₂ was added MeMgBr (3 M in Et₂O, 2.43 mL). The mixture was warmed to 20° C. and stirred for 7 h. The reaction was quenched by addition of aq. sat. NH₄Cl (1 mL) and concentrated under reduced pressure. The resulting residue was slurried with DCM (20 mL), filtered and the filtrate was concentrated under reduced pressure to give a mixture of the titled compounds LCMS: m/z=170.3 [M+H]⁺ and LCMS: m/z=156.2 [M+H]⁺.

### Intermediate 60

### cis-1-(1-methoxyethyl)-3-methyl-6-azabicyclo[3.1.1]heptane

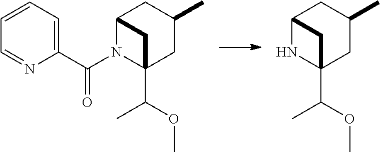

cis-1-(1-methoxyethyl)-3-methyl-6-azabicyclo[3.1.1]heptane: To a mixture of (cis-1-(1-methoxyethyl)-3-methyl-6-azabicyclo[3.1.1]heptan-6-yl)(pyridin-2-yl)methanone, Intermediate 57, (250 mg, 0.91 mmol) in THF (10 mL) was added MeMgBr (3 M in Et₂O, 1.52 mL) at 0° C. under N₂. The mixture was warmed to 20° C. and stirred for 7 h. The reaction was quenched by addition of aq. sat. NH₄Cl (1 mL) and concentrated under reduced pressure. The residue was slurried with DCM (20 mL), filtered and the filtrate was concentrated under reduce pressure to give the titled compound. LCMS: m/z=170.3 [M+H]⁺.

Intermediates 61 and 62

### (cis-1-(1-hydroxyethyl)-3-methyl-6-azabicyclo[3.1.1]heptan-6-yl)(pyridin-2-yl)methanone and (cis-1-(1-hydroxyethyl)-3-methyl-6-azabicyclo[3.1.1]heptan-6-yl)(pyridin-2-yl)methanone

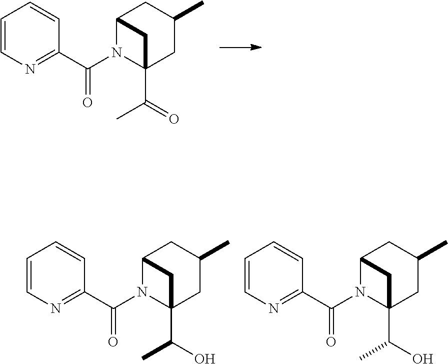

To a solution of 1-(cis-3-methyl-6-picolinoyl-6-azabicyclo[3.1.1]heptan-1-yl)ethan-1-one (5.5 g, 21.29 mmol) in MeOH (100 mL) was added NaBH₄ (1.61 g, 42.58 mmol) in portions at −30° C. The mixture was stirred at 0° C. for 2 h before quenching by the addition of sat. NH₄Cl (100 mL). The mixture was concentrated under reduced pressure to remove MeOH and the remaining aqueous phase was extracted with EtOAc (3×10 mL). The combined organic layers were washed with brine (10 mL), dried over anhydrous Na₂SO₄, filtered and concentrated under reduced pressure. The resulting residue was purified by silica gel column chromatography (PE:EtOAc=3:1 to 1:1) to give (cis-1-(1-hydroxyethyl)-3-methyl-6-azabicyclo[3.1.1]heptan-6-yl)(pyridin-2-yl)methanone Intermediate 61 as the first eluting isomer LCMS: m/z=261.3 [M+H]⁺ and (cis-1-(1-hydroxyethyl)-3-methyl-6-azabicyclo[3.1.1]heptan-6-yl)(pyridin-2-yl)methanone Intermediate 62 as the second eluting isomer. LCMS: m/z=261.3 [M+H]⁺.

### Intermediate 64

### methyl 2-(cis-3-methyl-6-picolinoyl-6-azabicyclo[3.1.1]heptan-1-yl)-2-oxoacetate

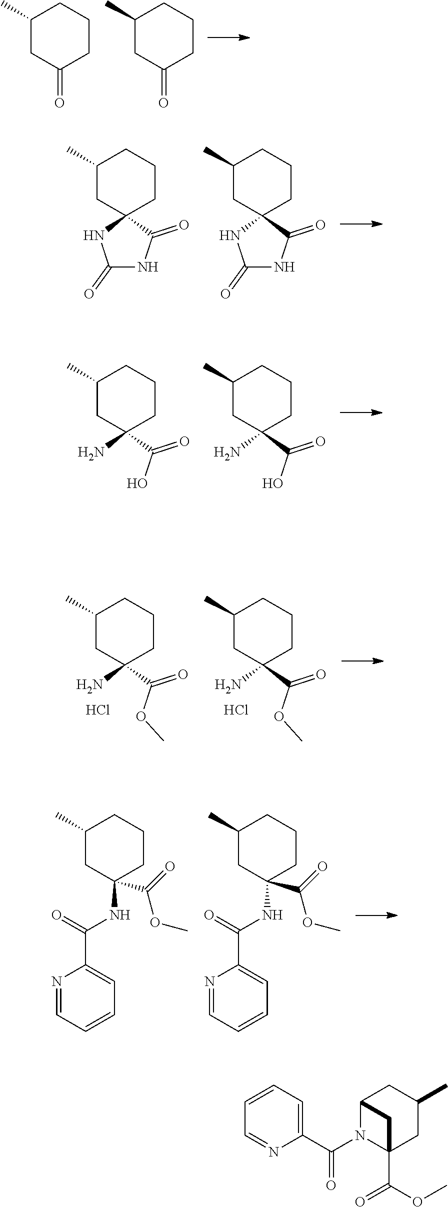

(5R,7R)-7-methyl-1,3-diazaspiro[4.5]decane-2,4-dione and (5S,7S)-7-methyl-1,3-diazaspiro[4.5]decane-2,4-dione: To a mixture of (R)-3-methylcyclohexan-1-one and (S)-3-methylcyclohexan-1-one (50 g, 445.76 mmol, 54.59 mL) in EtOH (250 mL) and H₂O (250 mL) was added (NH₄)₂CO₃ (128.49 g, 1.34 mol) and KCN (43.54 g, 669 mmol) at 20° C. under N₂. The mixture was heated to 65° C. and stirred for 3 h. The reaction mixture was filtered and the filter cake was washed with H₂O and dried under reduced pressure. The crude product was triturated with EtOH at 20° C. for 30 min, filtered, and the solid was dried under reduced pressure to give the titled compounds. LCMS: m/z=183.2 [M+H]⁺. [0416](1R,3R)-1-amino-3-methylcyclohexane-1-carboxylic acid and (1S,3S)-1-amino-3-methylcyclohexane-1-carboxylic acid: To a solution of (5R,7R)-7-methyl-1,3-diazaspiro[4.5]decane-2,4-dione and (5S,7S)-7-methyl-1,3-diazaspiro[4.5]decane-2,4-dione (78.5 g, 430.80 mmol) in H₂O (1000 mL) was added Ba(OH)₂ (738 g, 4.31 mol) at 25° C. The mixture was heated at 140° C. for 12 h in a 5 L autoclave. The reaction cooled to 0° C. and the pH was adjusted to pH=3 with 3 M H₂SO₄. The mixture was filtered through a celite pad and the filtrate was concentrated under reduced pressure to give the titled compounds. LCMS: m/z=158.2 [M+H]⁺.

methyl (1R,3R)-1-amino-3-methylcyclohexane-1-carboxylate hydrochloride and methyl (1S,3S)-1-amino-3-methylcyclohexane-1-carboxylate hydrochloride: To a mixture of (1R,3R)-1-amino-3-methylcyclohexane-1-carboxylic acid and (1S,3S)-1-amino-3-methylcyclohexane-1-carboxylic acid (60 g, 382 mmol) in MeOH (600 mL) was added SOCl₂ (227 g, 1.91 mol, 138.43 mL) at 0° C. under N₂. The mixture was heated at 75° C. for 12 h. The reaction mixture was concentrated under reduced pressure to give the titled compounds. LCMS: m/z=172.2 [M+H]⁺.

methyl (1R,3R)-3-methyl-1-(picolinamido)cyclohexane-1-carboxylate and methyl (1S,3S)-3-methyl-1-(picolinamido)cyclohexane-1-carboxylate: To a solution of methyl (1R,3R)-1-amino-3-methylcyclohexane-1-carboxylate hydrochloride and methyl (1S,3S)-1-amino-3-methylcyclohexane-1-carboxylate hydrochloride (24 g, 140 mmol) and picolinic acid (25.88 g, 210 mmol) in DCM (300 mL) was added DIEA (54.34 g, 420 mmol, 73.24 mL), DMAP (1.71 g, 14 mmol) and EDCI (40.30 g, 210 mmol) at 0° C. under N₂. The mixture was warmed to 25° C. and stirred for 16 h. The reaction mixture was diluted with H₂O (200 mL) and extracted with DCM (3×200 mL). The combined organic layers were washed with brine (200 mL), dried over anhydrous Na₂SO₄, filtered and concentrated under reduced pressure. The resulting residue was purified by silica gel column chromatography (PE:EtOAc=10:1 to 3:1) to give the titled compounds. LCMS: m/z=277.2 [M+H]⁺.

methyl 2-(cis-3-methyl-6-picolinoyl-6-azabicyclo[3.1.1]heptan-1-yl)-2-oxoacetate: To a solution of methyl (1R,3R)-3-methyl-1-(picolinamido)cyclohexane-1-carboxylate and methyl (1S,3S)-3-methyl-1-(picolinamido)cyclohexane-1-carboxylate (19 g, 68.76 mmol) in 1,1,2,2-tetrachloroetane (900 mL) was added Na₃PO₄ (33.82 g, 206 mmol, 33.82 mL), 1,2,3,4,5-pentafluoro-6-iodo-benzene (202.12 g, 688 mmol), AgOAc (34.43 g, 206 mmol, 10.56 mL), BQ (3.72 g, 34 mmol, 7.74 mL) and Pd(OAc)₂ (3.09 g, 13.75 mmol) at 25° C. under N₂. The mixture was heated to 145° C. and stirred for 16 h and then the reaction mixture was filtered through a celite pad. The filtrate was concentrated under reduced pressure and the resulting residue was purified by silica gel column chromatography (PE:EtOAc=10:1 to 3:1) to give the titled compound. LCMS: m/z=275.2 [M+H]⁺.

### Intermediate 67

### 3-chloro-5-(difluoromethoxy)pyridazine

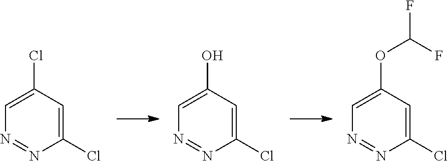 [SMILES: Clc1cnnc(Cl)c1 ; Oc1cnnc(Cl)c1 ; FC(F)Oc1cnnc(Cl)c1]

6-chloropyridazin-4-ol: To a solution of 3,5-dichloropyridazine (5 g, 33.56 mmol) in 1,4-dioxane (20 mL) and H₂O (20 mL) was added NaOH (2 g, 50 mmol) at 20° C. The mixture was heated to 100° C. and stirred for 12 h. The reaction mixture was adjusted to pH=4 by addition of diluted HCl at 0° C. The reaction mixture was filtered through a celite pad and the filtrate was concentrated under reduced pressure. The crude product was slurried with DCM:MeOH=10:1 (20 mL). The mixture was filtered and the filter cake was dried under reduced pressure to give the titled compound. LCMS: m/z=131.3, 133.3 [M+H]⁺.

3-chloro-5-(difluoromethoxy)pyridazine: To a solution of 6-chloropyridazin-4-ol (500 mg, 3.83 mmol) in DMF (12 mL) and H₂O (4 mL) was added sodium; 2-chloro-2,2-difluoro-acetate (1.34 g, 8.81 mmol) and K₂CO₃ (1.32 g, 9.58 mmol) at 25° C. The mixture was heated to 110° C. and stirred for 12 h. The reaction mixture was filtered through a celite pad and the filtrate was diluted with H₂O (10 mL) and extracted with EtOAc (3×5 mL). The combined organic layers were washed with brine (10 mL), dried over anhydrous Na₂SO₄, filtered and concentrated under reduced pressure. The resulting residue was purified by silica gel column chromatography (PE:EtOAc=10:1 to 1:1) to give the titled compound. LCMS: m/z=181.1, 183.2 [M+H]⁺.

### Intermediate 68

### 3-methylsulfanyl-6-(trifluoromethyl)-1,2,4-triazine and 3-methylsulfanyl-5-(trifluoromethyl)-1,2,4-triazine

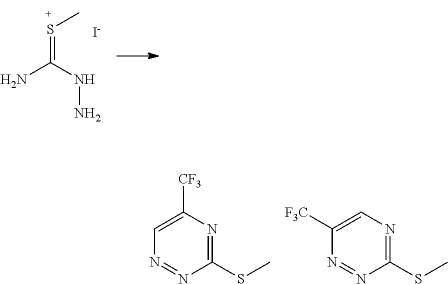

To a stirred solution of NaOAc (5.75 g, 70.12 mmol) in H₂O (50 mL) was added 3,3-dibromo-1,1,1-trifluoro-propan-2-one (20.43 g, 75.73 mmol). The solution was heated to 80° C. and stirred for 30 min, then allowed to cool to 20° C. before addition of solid (Z)-[amino(hydrazine)methylene]-methyl-sulfonium hydrochloride (5 g, 35.06 mmol). The mixture was stirred at 20° C. for 12 h. Then the reaction solution was extracted with EtOAc (3×40 ml) and the combined organic layers were washed with brine, dried over by Na₂SO₄, filtered and concentrated under reduced pressure. The crude product was purified by silica gel column chromatography (PE:EtOAc=100:1 to 10:1) to give the titled compounds. LCMS: m/z=196.1 [M+H]⁺.

### Intermediate 69

### 4-methyl-3-(5-(trifluoromethyl)-1,2,4-triazin-3-yl)aniline and 4-methyl-3-[6-(trifluoromethyl)-1,2,4-triazin-3-yl]aniline

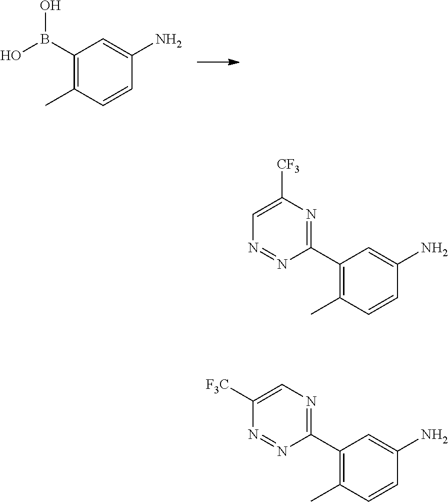 [SMILES: Cc1ccc(N)cc1B(O)O ; Cc1ccc(N)cc1-c1nncc(C(F)(F)F)n1 ; Cc1ccc(N)cc1-c1ncc(C(F)(F)F)nn1]

To a mixture of 3-methylsulfanyl-5-(trifluoromethyl)-1,2,4-triazine and 3-methylsulfanyl-6-(trifluoromethyl)-1,2,4-triazine (2 g, 10.25 mmol) and (5-amino-2-methyl-phenyl)boronic acid (1.86 g, 12.30 mmol) in 1,4-dioxane (20 mL) was added Pd(PPh₃)₄ (378 mg, 1.02 mmol) and thiophene-2-carbonyloxycopper; hydrate (4.71 g, 22.54 mmol) at 25° C. under N₂. The mixture was heated to 100° C. and stirred for 12 h. The reaction mixture was concentrated under reduced pressure and the crude product was purified by prep-HPLC (Waters Xbridge Prep OBD C18 150×40 mm×10 m; mobile phase: A: 10 mM NH₄HCO₃ in water, B: MeCN; B % in A: 25%-55%, 8 min) to give the titled compounds. LCMS: m/z=255.1 [M+H]⁺.

### Intermediate 70

### 3-chloro-5-(fluoromethoxy)pyridazine

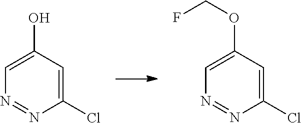 [SMILES: Oc1cnnc(Cl)c1 ; FCOc1cnnc(Cl)c1]

To a solution of 6-chloropyridazin-4-ol (500 mg, 3.83 mmol) in DMF (6 mL) was added fluoro(iodo)methane (1.41 g, 8.81 mmol) and K₂CO₃ (1.32 g, 9.58 mmol) at 25° C. under N₂. The mixture was heated to 110° C. and stirred for 2 h. Then the reaction mixture was filtered through a celite pad, the filtrate was diluted with H₂O (20 mL) and extracted with EtOAc (3×15 mL). The combined organic layers were washed with brine (2×10 mL), dried over anhydrous Na₂SO₄, filtered and concentrated under reduced pressure. The resulting residue was purified by silica gel column chromatography (PE:EtOAc=10:1 to 1:1) to give the titled compound. LCMS: m/z=163.1 [M+H]⁺.

### Intermediate 72

### cis-1-((difluoromethoxy)methyl)-3-methyl-6-azabicyclo[3.1.1]heptane

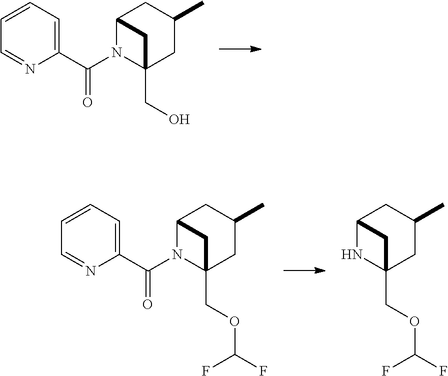

(cis-1-((difluoromethoxy)methyl)-3-methyl-6-azabicyclo[3.1.1]heptan-6-yl)(pyridine-2-yl)methanone: To a mixture of (cis-1-(hydroxymethyl)-3-methyl-6-azabicyclo[3.1.1]heptan-6-yl)(pyridine-2-yl)methanone (450 mg, 1.83 mmol) in DCM (7.5 mL) and H₂O (7.5 mL) at 25° C. under N₂ was added KHF₂ (1.14 g, 14.62 mmol) and the reaction was stirred for 10 min. Then (bromodifluoromethyl)trimethylsilane (1.11 g, 5.48 mmol) was added to the reaction mixture and stirred for 12 h. The reaction mixture was diluted with H₂O (10 mL) and extracted with DCM (3×5 mL). The combined organic layers were dried over anhydrous Na₂SO₄, filtered and concentrated under reduced pressure. The resulting residue was purified by silica gel column chromatography (PE:EtOAc=5:1 to 3:1) to give the titled compound. LCMS: m/z=297.1 [M+H]⁺.

cis-1-((difluoromethoxy)methyl)-3-methyl-6-azabicyclo[3.1.1]heptane: To a mixture of (cis-1-((difluoromethoxy)methyl)-3-methyl-6-azabicyclo[3.1.1]heptan-6-yl)(pyridin-2-yl)methanone (130 mg, 0.44 mol) in THF (5 mL) at 0° C. under N₂ was added MeMgBr (3 M in THF, 0.73 mL). The mixture was warmed to 25° C. and stirred for 1 h. The reaction was quenched by addition of aq. sat. NH₄Cl (1 mL) and concentrated under reduced pressure. Then the reaction was diluted with DCM (10 mL), filtered and the filtrate was concentrated under reduced pressure to give the titled compound. LCMS: m/z=192.2 [M+H]⁺.

### Example 1

### (1S,4S)-Methyl 5-((4-methyl-3-(pyridin-2-yl)phenyl)carbamoyl)-2,5-diazabicyclo[2.2.1]heptane-2-carboxylate

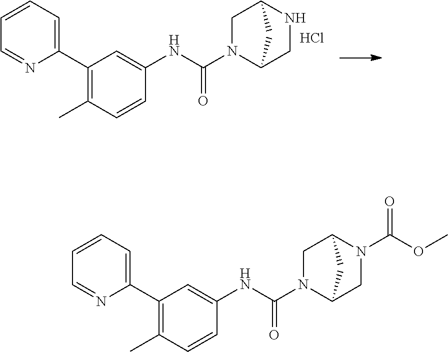 [SMILES: Cc1ccc(NC(=O)N2C[C@@H]3C[C@H]2CN3)cc1-c1ccccn1 ; Cl ; COC(=O)N1C[C@@H]2C[C@H]1CN2C(=O)Nc1ccc(C)c(-c2ccccn2)c1]

To a mixture of (1S,4S)—N-(4-methyl-3-(pyridin-2-yl)phenyl)-2,5-diazabicyclo[2.2.1]heptane-2-carboxamide hydrochloride (100 mg, 0.26 mmol) in DCM (2 mL) was added TEA (80 mg, 0.79 mmol) and methyl carbonochloridate (27 mg, 0.29 mmol) at 0° C. under N₂. The mixture was stirred at 25° C. for 0.5 h. The reaction mixture was diluted with H₂O (5 mL) and extracted with DCM (3×3 mL). The combined organic layers were dried over anhydrous Na₂SO₄, filtered and concentrated under reduced pressure. The resulting residue was purified by prep-TLC (SiO₂, PE:EtOAc=0:1) to give the titled compound. ¹H NMR (400 MHz, CDCl₃): δ 8.67-8.61 (m, 1H), 7.72 (td, J=2.0, 7.6 Hz, 1H), 7.42-7.33 (m, 3H), 7.25-7.21 (m, 1H), 7.17 (d, J=8.8 Hz, 1H), 6.51 (s, 1H), 4.75-4.50 (m, 2H), 3.73-3.64 (m, 3H), 3.56-3.33 (m, 4H), 2.29 (s, 3H), 1.82 (br s, 2H). LCMS: m/z=367.2 [M+H]⁺.

### Example 2

### N-(4-Methyl-3-(pyridin-2-yl)phenyl)-3-(1,1,1-trifluoropropan-2-yl)-3,6-diazabicyclo[3.1.1]heptane-6-carboxamide

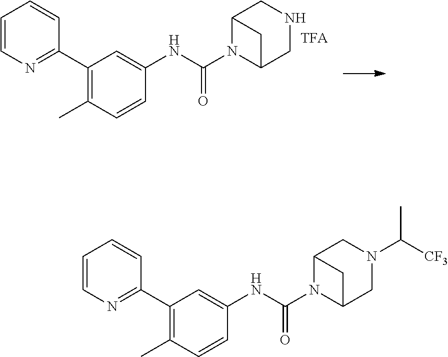 [SMILES: Cc1ccc(NC(=O)N2C3CNCC2C3)cc1-c1ccccn1 ; Cc1ccc(NC(=O)N2C3CC2CN(C(C)C(F)(F)F)C3)cc1-c1ccccn1 ; "TFA"]

To a mixture of N-(4-methyl-3-(pyridin-2-yl)phenyl)-3,6-diazabicyclo[3.1.1]heptane-6-carboxamide trifluoroacetate (300 mg, 0.56 mmol) and DIEA (361 mg, 2.80 mmol) in THF (5 mL) was added crude trifluoropropan-2-yl 1,1,2,2,3,3,4,4,4-nonafluorobutane-1-sulfonate (328 mg, 0.67 mmol) at 0° C. under N₂. The mixture was stirred at 25° C. for 2 h. The reaction mixture was diluted with H₂O (5 mL) and extracted with EtOAc (3×5 mL). The combined organic layers were dried over anhydrous Na₂SO₄, filtered and concentrated under reduced pressure. The resulting residue was purified by prep-HPLC (column: Waters Xbridge BEH C18 100×30 mm×10 m; mobile phase: A: 10 mM NH₄HCO₃ in water, B: MeCN; B % in A: 25%-50%, 10 min) to give the titled compound as a mixture of enantiomers. ¹H NMR (400 MHz, CDCl₃): δ 8.68 (d, J=4.8 Hz, 1H), 7.74 (td, J=1.6, 7.6 Hz, 1H), 7.47 (dd, J=2.4, 8.4 Hz, 1H), 7.42 (d, J=7.6 Hz, 1H), 7.33 (d, J=2.0 Hz, 1H), 7.27-7.23 (m, 1H), 7.21 (d, J=8.4 Hz, 1H), 6.20 (s, 1H), 4.21-4.14 (m, 2H), 3.68-3.57 (m, 1H), 3.55-3.45 (m, 1H), 3.28-3.16 (m, 1H), 3.10 (d, J=10.0 Hz, 1H), 2.86 (d, J=10.4 Hz, 1H), 2.61-2.51 (m, 1H), 2.32 (s, 3H), 1.57 (d, J=8.0 Hz, 1H), 1.22 (d, J=6.8 Hz, 3H). LCMS: m/z=405.2 [M+H]⁺.

### Example 3

### N-(2-Fluoro-4-methyl-5-(pyridin-2-yl)phenyl)-3-(2,2,2-trifluoroethyl)-3,6-diazabicyclo[3.1.1]heptane-6-carboxamide

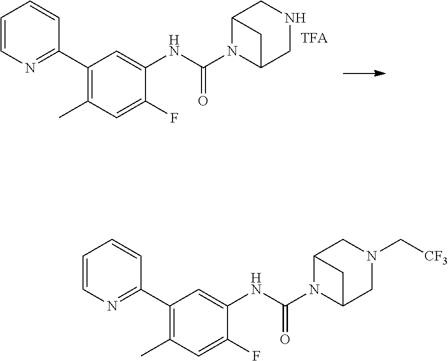 [SMILES: Cc1cc(F)c(NC(=O)N2C3CNCC2C3)cc1-c1ccccn1 ; Cc1cc(F)c(NC(=O)N2C3CC2CN(CC(F)(F)F)C3)cc1-c1ccccn1 ; "TFA"]

To a solution of N-(2-fluoro-4-methyl-5-(pyridin-2-yl)phenyl)-3,6-diazabicyclo[3.1.1]heptane-6-carboxamide trifluoroacetate (100 mg, 0.23 mmol) and DIEA (147 mg, 1.14 mmol) in THF (2 mL) was added 2,2,2-trifluoroethyl trifluoromethanesulfonate (63 mg, 0.30 mmol) at 0° C. The mixture was stirred at 25° C. for 12 h. The mixture was concentrated under reduced pressure. The resulting residue was purified by prep-HPLC (column: Phenomenex C18 75×30 mm×3 m; mobile phase: A: 10 mM NH₄HCO₃ in water; B: MeCN; B % in A: 25%-60%, 8 min) to give the titled compound. ¹H NMR (400 MHz, CDCl₃): δ 8.66 (br d, J=4.8 Hz, 1H), 8.13 (d, J=8.4 Hz, 1H), 7.73 (td, J=1.6, 7.6 Hz, 1H), 7.42 (d, J=8.0 Hz, 1H), 7.25-7.22 (m, 1H), 7.00 (d, J=12.0 Hz, 1H), 6.20 (br s, 1H), 4.24 (d, J=6.0 Hz, 2H), 3.45 (d, J=10.4 Hz, 2H), 3.23-3.14 (m, 4H), 2.62-2.56 (m, 1H), 2.32 (s, 3H), 1.78 (d, J=8.0 Hz, 1H). LCMS: m/z=409.1 [M+H]⁺.

### Example 4

### N-(4-methyl-3-(pyridin-2-yl)phenyl)-3-(2,2,2-trifluoroethyl)-3,6-diazabicyclo[3.1.1]heptane-6-carboxamide

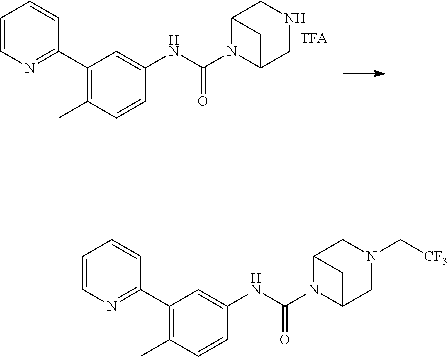 [SMILES: Cc1ccc(NC(=O)N2C3CNCC2C3)cc1-c1ccccn1 ; Cc1ccc(NC(=O)N2C3CC2CN(CC(F)(F)F)C3)cc1-c1ccccn1 ; "TFA"]

To a mixture of N-(4-methyl-3-(pyridin-2-yl)phenyl)-3,6-diazabicyclo[3.1.1]heptane-6-carboxamide trifluoroacetate (300 mg, 0.56 mmol) and 2,2,2-trifluoroethyl trifluoromethanesulfonate (156 mg, 0.67 mmol) in THF (5 mL) was added DIEA (217 mg, 1.68 mmol) at 0° C. under N₂. The mixture was stirred at 25° C. for 2 h. The reaction mixture was diluted with H₂O (10 mL) and extracted with EtOAc (3×5 mL). The combined organic layers were dried over anhydrous Na₂SO₄, filtered and concentrated under reduced pressure. The resulting residue was purified by prep-HPLC (column: Phenomenex C18 80×40 mm×3 m; mobile phase: A: 10 mM NH₄HCO₃ in water, B: MeCN; B % in A: 25%-45%, 8 min) to give the titled compound. ¹H NMR (400 MHz, CDCl₃): δ 8.69 (br d, J=4.8 Hz, 1H), 7.75 (t, J=7.2 Hz, 1H), 7.49-7.40 (m, 2H), 7.35 (d, J=2.0 Hz, 1H), 7.27-7.19 (m, 2H), 6.14 (s, 1H), 4.18 (d, J=6.0 Hz, 2H), 3.45-3.42 (m, 2H), 3.25-3.09 (m, 4H), 2.55 (q, J=6.4 Hz, 1H), 2.32 (s, 3H), 1.76 (d, J=8.4 Hz, 1H). LCMS: m/z=391.2 [M+H]⁺.

### Example 5

### 3-(2,2-difluoroethyl)-N-(4-methyl-3-(pyridin-2-yl)phenyl)-3,6-diazabicyclo[3.1.1]heptane-6-carboxamide

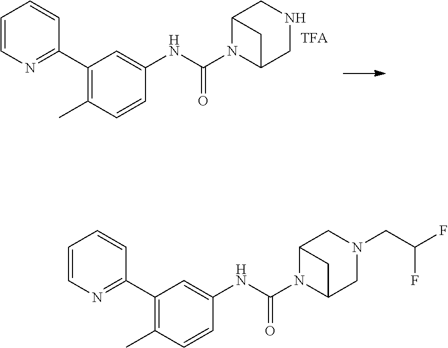 [SMILES: Cc1ccc(NC(=O)N2C3CNCC2C3)cc1-c1ccccn1 ; Cc1ccc(NC(=O)N2C3CC2CN(CC(F)F)C3)cc1-c1ccccn1 ; "TFA"]

To a mixture of N-(4-methyl-3-(pyridin-2-yl)phenyl)-3,6-diazabicyclo[3.1.1]heptane-6-carboxamide trifluoroacetate (200 mg, 0.37 mmol) and DIEA (144.56 mg, 1.12 mmol) in THF (5 mL) was added 2,2-difluoroethyl trifluoromethanesulfonate (95.80 mg, 0.45 mmol) at 0° C. under N₂. The mixture was stirred at 25° C. for 2 h. The reaction mixture was diluted with H₂O (5 mL) and extracted with EtOAc (3×5 mL). The combined organic layers were dried over anhydrous Na₂SO₄, filtered and concentrated under reduced pressure. The resulting residue was purified by prep-HPLC (column: Phenomenex Luna C18 75×30 mm×3 m; mobile phase: A: 10 mM FA in water, B: MeCN; B in A: 5%-25%, 8 min) to give the titled compound. ¹H NMR (400 MHz, CDCl₃): δ 8.68 (br d, J=4.8 Hz, 1H), 7.75 (td, J=1.6, 7.6 Hz, 1H), 7.49-7.41 (m, 2H), 7.36 (d, J=2.4 Hz, 1H), 7.26-7.20 (m, 2H), 6.16 (s, 1H), 5.82 (tt, J=4.4, 56.0 Hz, 1H), 4.18 (d, J=6 Hz, 2H), 3.78-3.01 (m, 4H), 2.94 (td, J=3.6, 14.8 Hz, 2H), 2.57-2.47 (m, 1H), 2.32 (s, 3H), 1.81 (d, J=8.4 Hz, 1H). LCMS: m/z=373.2 [M+H]⁺.

### Example 6

### 3-Cyclobutyl-N-(4-methyl-3-(pyridin-2-yl)phenyl)-3,6-diazabicyclo[3.1.1]heptane-6-carboxamide

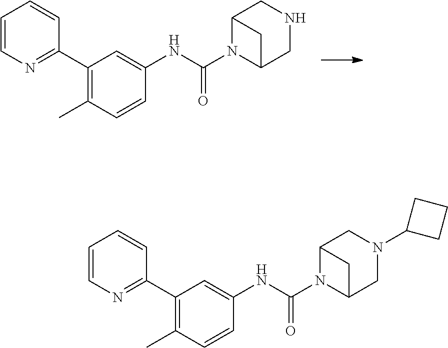 [SMILES: Cc1ccc(NC(=O)N2C3CNCC2C3)cc1-c1ccccn1 ; Cc1ccc(NC(=O)N2C3CC2CN(C2CCC2)C3)cc1-c1ccccn1]

To a mixture of cyclobutanone (57 mg, 0.81 mmol) and N-(4-methyl-3-(pyridin-2-yl)phenyl)-3,6-diazabicyclo[3.1.1]heptane-6-carboxamide (100 mg, 0.32 mmol) in MeOH (2 mL) was added AcOH (58 mg, 0.97 mmol) at 25° C. under N₂. The mixture was stirred at 25° C. for 30 min, then NaBH₃CN (41 mg, 0.65 mmol) was added to the mixture and stirred at 25° C. for 12 h. The mixture was adjusted to pH=7-8 with sat. aq. Na₂CO₃ and extracted with EtOAc (3×5 mL). The combined organic layers were dried over anhydrous Na₂SO₄, filtered and concentrated under reduced pressure. The resulting residue was purified by prep-HPLC (column: Waters Xbridge BEH C18 100×30 mm×10 m; mobile phase: A: 10 mM NH₄HCO₃ in water, B: MeCN; B % in A: 25%-55%, 8 min) to give the titled compound. ¹H NMR (400 MHz, CDCl₃): δ 8.68 (d, J=4.8 Hz, 1H), 7.74 (td, J=1.6, 7.6 Hz, 1H), 7.47 (dd, J=2.4, 8.4 Hz, 1H), 7.43 (d, J=7.6 Hz, 1H), 7.35 (d, J=2.4 Hz, 1H), 7.26-7.23 (m, 1H), 7.21 (d, J=8.0 Hz, 1H), 6.18 (br s, 1H), 4.19 (d, J=5.6 Hz, 2H), 3.15-3.03 (m, 3H), 2.82 (d, J=11.2 Hz, 2H), 2.49-2.42 (m, 1H), 2.32 (s, 3H), 2.00-1.92 (m, 2H), 1.92-1.84 (m, 3H), 1.69-1.60 (m, 2H). LCMS: m/z=363.3 [M+H]⁺.

### Example 7

### 3-Ethyl-N-(4-methyl-3-(pyridin-2-yl)phenyl)-3,6-diazabicyclo[3.1.1]heptane-6-carboxamide

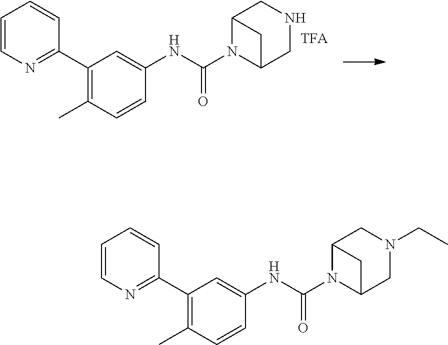 [SMILES: Cc1ccc(NC(=O)N2C3CNCC2C3)cc1-c1ccccn1 ; CCN1CC2CC(C1)N2C(=O)Nc1ccc(C)c(-c2ccccn2)c1 ; "TFA"]

To a mixture of N-[4-methyl-3-(2-pyridyl)phenyl]-3,6-diazabicyclo[3.1.1]heptane-6-carboxamide trifluoroacetate (300 mg, 0.97 mmol) and DIEA (377 mg, 2.92 mmol) in THF (5 mL) was added iodoethane (182 mg, 1.17 mmol) at 0° C. under N₂. The mixture was stirred at 25° C. for 2 h. The reaction mixture was diluted with H₂O (5 mL) and extracted with EtOAc (3×3 mL). The combined organic layers were dried over anhydrous Na₂SO₄, filtered and concentrated under reduced pressure. The resulting residue was purified by prep-HPLC (column: Phenomenex C18 80×40 mm×3 m; mobile phase: A: 10 mM NH₄HCO₃ in water, B: MeCN; B % in A: 30%-50% over 8 min) to give the titled compound. ¹H NMR (400 MHz, CDCl₃): δ 8.68 (d, J=4.8 Hz, 1H), 7.73 (td, J=1.6, 7.6 Hz, 1H), 7.51-7.39 (m, 2H), 7.36 (d, J=2.0 Hz, 1H), 7.26-7.22 (m, 1H), 7.20 (d, J=8.4 Hz, 1H), 6.18 (s, 1H), 4.19 (br d, J=5.6 Hz, 2H), 3.11-2.88 (m, 4H), 2.56 (q, J=7.2 Hz, 2H), 2.44 (q, J=6.4 Hz, 1H), 2.31 (s, 3H), 1.89 (d, J=7.6 Hz, 1H), 1.04 (t, J=7.2 Hz, 3H). LCMS: m/z=337.2 [M+H]⁺.

### Example 8

### N-[4-Methyl-3-(2-pyridyl)phenyl]-11-azatricyclo[6.2.1.02,7]undeca-2,4,6-triene-11-carboxamide

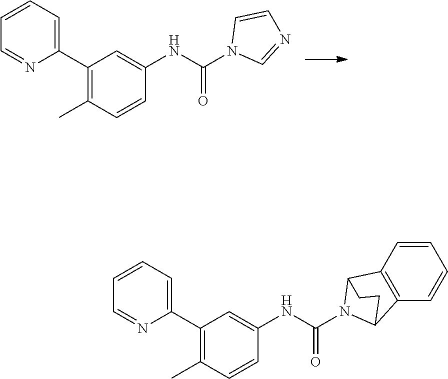 [SMILES: Cc1ccc(NC(=O)n2ccnc2)cc1-c1ccccn1 ; Cc1ccc(NC(=O)N2C3CCC2c2ccccc23)cc1-c1ccccn1]

[4-methyl-3-(2-pyridyl)phenyl]imidazole-1-carboxamide (40.0 mg, 0.14 mmol) was suspended in THF (1.4 mL) and was treated with 11-azatricyclo[6.2.1.0,2,7]undeca-2,4,6-triene hydrochloride (78.33 mg, 0.43 mmol). The reaction mixture was stirred at 65° C. for 0.5 h and subsequently concentrated to dryness. The crude reaction mixture was purified by column chromatography 20-70% EtOAc/hex to give the titled compound. LCMS: m/z=356.2 [M+H]⁺. ¹H-NMR (400 MHz, CDCl₃): δ 8.66 (d, J=4.3 Hz, 1H), 7.72 (td, J=7.7, 1.7 Hz, 1H), 7.43-7.40 (m, 3H), 7.28-7.24 (m, 3H), 7.17 (dt, J=5.8, 3.1 Hz, 3H), 6.76 (s, 1H), 5.21 (t, J=2.0 Hz, 2H), 2.31 (s, 3H), 2.24-2.21 (m, 2H), 1.39-1.35 (m, 2H).

### Example 9

### Methyl 6-[[4-methyl-3-(2-pyridyl)phenyl]carbamoyl]-6-azabicyclo[3.1.1]heptane-1-carboxylate

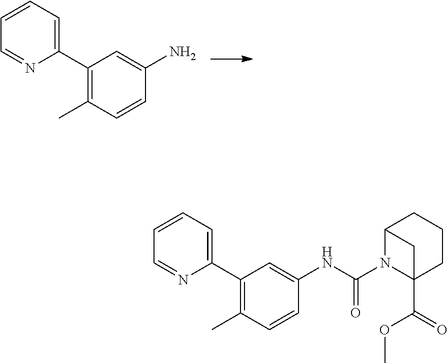 [SMILES: Cc1ccc(N)cc1-c1ccccn1 ; COC(=O)C12CCCC(C1)N2C(=O)Nc1ccc(C)c(-c2ccccn2)c1]

To a solution of 4-methyl-3-(2-pyridyl)aniline (50.0 mg, 0.3 mmol) in THF (2.7 mL) at −20° C. was added CDI (52.8 mg, 0.3 mmol). This was allowed to warm to room temperature over 0.5 h. Then a solution of methyl 6-azabicyclo[3.1.1]heptane-1-carboxylate trifluoroacetate (73.0 mg, 0.3 mmol) and TEA (0.08 mL, 0.5 mmol) in 0.5 mL DCM was added and the reaction mixture was heated at 50° C. for 0.5 h. The reaction mixture was concentrated and purified by reverse-phase HPLC to give the titled product as a mixture of enantiomers. LCMS: m/z=366.2. ¹H-NMR (400 MHz, CDCl3): δ 8.95 (s, 1H), 8.75 (dd, J=5.1, 0.9 Hz, 1H), 7.89 (t, J=7.3 Hz, 1H), 7.57-7.50 (m, 3H), 7.38 (t, J=6.4 Hz, 1H), 7.23 (d, J=8.2 Hz, 1H), 4.18-4.15 (m, 1H), 3.85 (s, 3H), 2.65-2.61 (m, 2H), 2.43-2.37 (m, 1H), 2.34 (s, 3H), 1.99-1.96 (m, 2H), 1.85-1.80 (m, 1H), 1.75 (d, J=8.7 Hz, 1H), 1.67-1.61 (m, 1H).

### Example 10

### trans-3-Methyl-N-(4-methyl-3-(pyridin-2-yl)phenyl)-6-azabicyclo[3.1.1]heptane-6-carboxamide

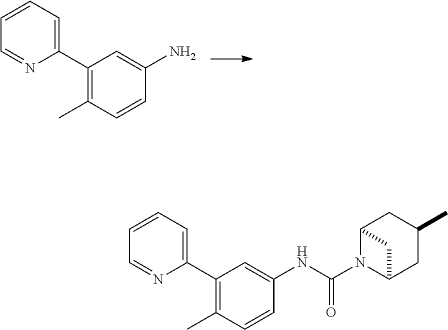

To a solution of CDI (53 mg, 0.325 mmol) in DCM (2 mL) was added dropwise a solution of 4-methyl-3-(2-pyridyl)aniline (50 mg, 0.27 mmol) in DCM (2 mL) over 10 min at −20° C. under N₂. The mixture was stirred at 20° C. for 1 h. Then a mixture of trans-3-methyl-6-azabicyclo[3.1.1]heptane (59 mg, 0.53 mmol) and TEA (65 mg, 0.65 mmol) in DCM (2 mL) was added to the above solution at 25° C. The mixture was stirred at 25° C. for 12 h. The reaction mixture was diluted with H₂O (2 mL) and extracted with DCM (3×3 mL). The combined organic layers were dried over anhydrous Na₂SO₄, filtered and concentrated under reduced pressure. The crude product was purified by prep-HPLC (column: Phenomenex C18 75×30 mm×3 m; mobile phase: A: 10 mM NH₄HCO₃ in water, B: MeCN; B in A: 25%-55%, 8 min) to give the titled product. ¹H NMR (400 MHz, CDCl₃): δ 8.68 (d, J=4.4 Hz, 1H), 7.73 (td, J=1.6, 7.6 Hz, 1H), 7.50 (dd, J=2.4, 8.4 Hz, 1H), 7.43 (d, J=7.8 Hz, 1H), 7.37 (d, J=2.4 Hz, 1H), 7.26-7.22 (m, 1H), 7.21 (d, J=8.4 Hz, 1H), 6.05 (s, 1H), 4.20 (d, J=6.0 Hz, 2H), 2.39-2.35 (m, 1H), 2.32-2.26 (m, 4H), 1.93 (br d, J=8.6 Hz, 4H), 1.67 (d, J=8.6 Hz, 1H), 0.98 (d, J=6.8 Hz, 3H). LCMS: m/z=322.2 [M+H]⁺.

### Example 11

### cis-3-Methyl-N-(4-methyl-3-(pyridin-2-yl)phenyl)-6-azabicyclo[3.1.1]heptane-6-carboxamide

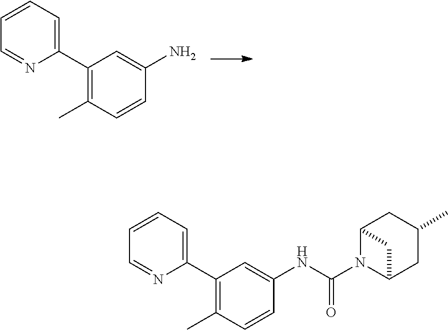

To a mixture of CDI (211 mg, 1.30 mmol) in DCM (6 mL) was added dropwise a solution of 4-methyl-3-(2-pyridyl)aniline (200 mg, 1.09 mmol) in DCM (6 mL) at −20° C. under N₂. The mixture was stirred at −20° C. for 1 h. Then a mixture of cis-3-methyl-6-azabicyclo[3.1.1]heptane (80 mg, 0.72 mmol) and TEA (218 mg, 2.16 mmol) in DCM (3 mL) was added to the above mixture at 25° C. The mixture was stirred at 50 SC for 0.5 h. The reaction mixture was diluted with H₂ (5 mL) and extracted with EtOAc (3×3 mL). The combined organic layers were dried over anhydrous Na₂SO₄, filtered and concentrated under reduced pressure. The resulting residue was purified by prep-HPLC (column: Phenomenex C18 80×40 mm×3 in; mobile phase: A: 10 mM NH₄HCO₃ in water, B: MeCN; B % in A: 25%-55%, 8 min) to give the titled compound. ¹H NMR (400 MHz, CDCl₃): δ 8.69 (d, J=4.4 Hz, H), 7.74 (td, J=1.6, 7.6 Hz, 1H), 7.48 (dd, J=2.4, 8.0 Hz, H), 7.44 (d, J=8.0 Hz, JH), 7.39-7.37 (m, 1H), 7.26-7.19 (i, 2H), 6.09 (s, 1H), 4.24-4.15 (i, 2H), 2.67-2.53 (m, 3H), 2.32 (s, 3H), 2.14-2.00 (m, 1H), 1.39-1.29 (m, 2H), 1.07 (d, J=8.4 Hz, 1H), 1.03 (d, J=6.8 Hz, 3H). LCMS: mz=322.2 [M+H]⁺.

The following compounds as shown in Table 1 were, or can be, made via similar procedures as those described above. Example 12 was prepared from a commercially available amine with unknown stereochemistry. Only one racemate was obtained but unknown if the final compound is the cis- or trans-isomer.

| Ex. | Structure | Name | NMR | LCMS |
|---|---|---|---|---|
| 12 | 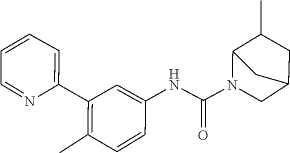 [SMILES: Cc1ccc(NC(=O)N2CC3CC(C)C2C3)cc1-c1ccccn1] single diastereomer | 6-methyl-N-(4- methyl-3- (pyridin-2- yl)phenyl)-2- azabicyclo[2.2.1] heptane-2- carboxamide | ¹H NMR (400 MHz, CD₃OD):δ 8.67 (d, J = 4.8 Hz, 1H), 7.74 (td, J = 1.6 Hz, 7.6 Hz, 1H), 7.50-7.43 (m, 2H), 7.40 (d, J = 2.4 Hz, 1H), 7.26-7.22 (m, 1H), 7.20 (d, J = 8.0 Hz, 1H), 6.10 (s, 1H), 4.21 (s, 1H), 3.45-3.41 (m, 1H), 2.98 (d, J = 8.0 Hz, 1H), 2.59 (s, 1H), 2.32 (s, 3H), | m/z = 322.2 [M + H]⁺ |
| | | | 2.22-2.11 (m, 1H), | |
| | | | 1.99-1.98 (m, 1H), | |
| | | | 1.70-1.67 (m, 1H), | |
| | | | 1.58-1.57 (m, 1H), 1.03 | |
| | | | (d, J = 6.8 Hz, 3H), | |
| | | | 0.88-0.79 (m, 1H). | |
| 13 | 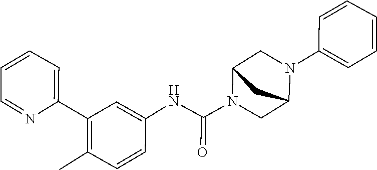 [SMILES: Cc1ccc(NC(=O)N2C[C@@H]3C[C@H]2CN3c2ccccc2)cc1-c1ccccn1] | (1S,4S)-N-(4- methyl-3- (pyridin-2- yl)phenyl)-5- phenyl-2,5- diazabicyclo [2.2.1]heptane-2- carboxamide | ¹H NMR (400 MHz, CD₃OD): δ 8.56 (d, J = 4.8 Hz, 1H), 7.91 (td, J = 1.6, 7.6 Hz, 1H), 7.49 (d, J = 8.0 Hz, 1H), 7.41-7.34 (m, 3H), 7.19-7.15 (m, 3H), 6.68-6.61 (m, 3H), 4.58 (s, 2H), 3.64 (dd, J = 1.6, 8.8 Hz, 1H), 3.55 (s, 2H), 3.20 (d, J = 8.8 | m/z = 385.2 [M + H]⁺ |
| | | | Hz, 1H), 2.20 (s, 3H), | |
| | | | 2.11-1.99 (m, 2H) | |
| 14 | 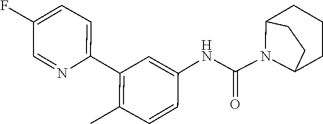 [SMILES: Cc1ccc(NC(=O)N2C3CCCC2CC3)cc1-c1ccc(F)cn1] | N-(3-(5- fluoropyridin-2- yl)-4- methylphenyl)-8- azabicyclo[3.2.1] octane-8- carboxamide | ¹H-NMR (400 MHz, CDCl₃): δ 8.54 (s, 1H), 7.46-7.44 (m, 2H), 7.43-7.42 (m, 1H), 7.41-7.40 (m, 1H), 7.21-7.19 (m, 1H), 6.32 (s, 1H), 4.25 (br s, 2H), 2.30 (s, 3H), 2.10-2.02 | m/z = 340.2 [M + H]⁺ |
| | | | (m, 2H), 1.93-1.80 (m, | |
| | | | 3H), 1.80-1.74 (m, 2H), | |
| | | | 1.65-1.60 (m, 1H), 1.49 | |
| | | | (br d, J = 10.4 Hz, 2H) | |
| 15 | 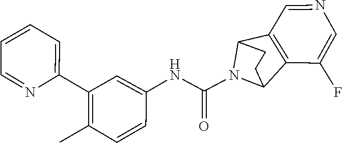 [SMILES: Cc1ccc(NC(=O)N2C3CCC2c2c(F)cncc23)cc1-c1ccccn1] | 4-fluoro-N-(4- methyl-3- (pyridin-2- yl)phenyl)- 5,6,7,8- tetrahydro-5,8- epiminoisoquinoline- 9-carboxamide | ¹H NMR (400 MHz, CDCl₃): δ 8.65 (d, J = 4.8 Hz, 1H), 8.29 (s, 2H), 7.73 (td, J = 1.6, 7.6 Hz, 1H), 7.39 (d, J = 8.0 Hz, 1H), 7.35-7.30 (m, 2H), 7.26-7.21 (m, 2H), 7.17-7.14 (m, 1H), 5.48-5.44 (m, 1H), 5.29 (br s, 1H), 2.28 (s, 3H), | m/z = 375.2 [M + H]⁺ |
| | mixture of enantiomers | | 2.23 (d, J = 9.6 Hz, 2H), | |
| | | | 1.42-1.31 (m, 2H) | |
| 16 | 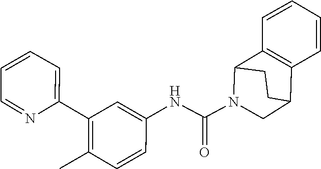 [SMILES: Cc1ccc(NC(=O)N2CC3CCC2c2ccccc23)cc1-c1ccccn1] mixture of enantiomers | N-(4-methyl-3- (pyridin-2- yl)phenyl)- 1,2,3,4- tetrahydro-1,4- (epiminomethano) naphthalene-10- carboxamide | ¹H NMR (400 MHz, CDCl₃): δ 8.66 (d, J = 4.8 Hz, 1H), 7.72 (td, J = 1.6, 7.6 Hz, 1H), 7.44-7.36 (m, 3H), 7.32-7.29 (m, 1H), 7.28-7.27 (m, 2H), 7.27-7.21 (m, 2H), 7.19-7.16 (m, 1H), 6.10 (s, 1H), 5.50 (br s, 1H), 3.60 (dd, J = 2.0, 8.0 Hz, 1H), 3.36-3.30 (m, 1H), 3.24-3.19 (m, 1H), | m/z = 370.2 [M + H]⁺ |
| | | | 2.31 (s, 3H), 2.29-2.23 | |
| | | | (m, 1H), 1.97-1.90 (m, | |
| | | | 1H), 1.61-1.52 (m, 2H) | |
| 17 | 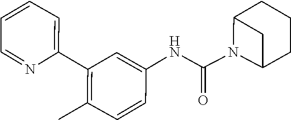 [SMILES: Cc1ccc(NC(=O)N2C3CCCC2C3)cc1-c1ccccn1] | N-(4-methyl-3- (pyridin-2- yl)phenyl)-6- azabicyclo[3.1.1] heptane-6- carboxamide | ¹H NMR (400 MHz, CDCl₃): δ 8.76 (d, J = 4.8 Hz, 1H), 7.81 (td, J = 1.6, 7.6 Hz, 1H), 7.57-7.54 (m, 1H), 7.51-7.49 (m, 1H), 7.46 (d, J = 2.0 Hz, 1H), 7.34-7.29 (m, 2H), 6.1 | m/z = 308.2 [M + H]⁺ |
| | | | (s, 1H), 4.28 (br d, J = | |
| | | | 6.0 Hz, 2H), 2.65-2.57 | |
| | | | (m, 1H), 2.50-2.41 (m, | |
| | | | 2H), 2.39 (s, 3H), 2.09- | |
| | | | 1.95 (m, 1H), 1.86-1.76 | |
| | | | (m, 3H), 1.56 (d, J = 8.8 | |
| | | | Hz, 1H) | |
| 18 | 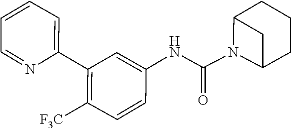 [SMILES: O=C(Nc1ccc(C(F)(F)F)c(-c2ccccn2)c1)N1C2CCCC1C2] | N-(3-(pyridin-2- yl)-4- (trifluoromethyl) phenyl)-6- azabicyclo[3.1.1] heptane-6- carboxamide | ¹H NMR (400 MHz, CDCl₃): δ 8.67 (d, J = 4.4 Hz, 1H), 7.79-7.72 (m, 2H), 7.70-7.65 (m, 1H), 7.49-7.43 (m, 2H), 7.36-7.28 (m, 1H), 6.25 (s, 1H), 4.25 (d, J = 6.0 Hz, 2H), 2.60-2.52 (m, | m/z = 362.2 [M + H]⁺ |
| | | | 1H), 2.42-2.30 (m, 2H), | |
| | | | 2.04-1.90 (m, 1H), | |
| | | | 1.81-1.71 (m, 3H), 1.53 | |
| | | | (d, J = 8.6 Hz, 1H) | |
| 19 | 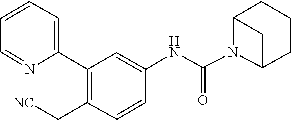 [SMILES: N#CCc1ccc(NC(=O)N2C3CCCC2C3)cc1-c1ccccn1] | N-(4- (cyanomethyl)-3- (pyridin-2- yl)phenyl)-6- azabicyclo[3.1.1] heptane-6- carboxamide | ¹H NMR (400 MHz, CDCl₃): δ 8.68 (d, J = 4.4 Hz, 1H), 7.80 (td, J = 1.6, 7.6 Hz, 1H), 7.73 (d, J = 2.0 Hz, 1H), 7.55 (d, J = 8 Hz, 1H), 7.48- 7.45 (m, 1H), 7.41-7.37 (m, 1H), 7.32-7.28 (m, | m/z = 333.2 [M + H]⁺ |
| | | | 1H), 6.08 (s, 1H), 4.26 | |
| | | | (br d, J = 6.0 Hz, 2H), | |
| | | | 4.00 (s, 2H), 2.62-2.51 | |
| | | | (m, 1H), 2.45-2.31 (m, | |
| | | | 2H), 2.07-1.91 (m, 1H), | |
| | | | 1.83-1.70 (m, 3H), 1.52 | |
| | | | (d, J = 8.8 Hz, 1H) | |
| 20 | 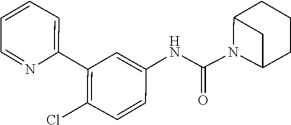 [SMILES: O=C(Nc1ccc(Cl)c(-c2ccccn2)c1)N1C2CCCC1C2] | N-(4-chloro-3- (pyridin-2- yl)phenyl)-6- azabicyclo[3.1.1] heptane-6- carboxamide | ¹H NMR (400 MHz, CDCl₃): δ 8.70 (d, J = 4.8 Hz, 1H), 7.77-7.70 (m, 1H), 7.70-7.67 (m, 2H), 7.50 (d, J = 2.8 Hz, 1H), 7.39 (d, J = 8.8 Hz, 1H), 7.32-7.29 (m, 1H), 6.22 (s, 1H), 4.22 (d, J = | m/z = 328.2 [M + H]⁺ |
| | | | 6.0 Hz, 2H), 2.60- | |
| | | | 2.49 (m, 1H), 2.43-2.29 | |
| | | | (m, 2H), 2.03-1.89 (m, | |
| | | | 1H), 1.73-1.78 (m, 3H), | |
| | | | 1.51 (d, J = 8.4 Hz, 1H) | |
| 21 | 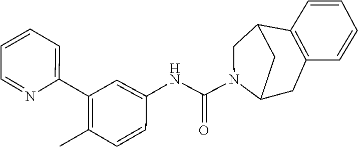 [SMILES: Cc1ccc(NC(=O)N2CC3CC2Cc2ccccc23)cc1-c1ccccn1] | N-(4-methyl-3- (pyridin-2- yl)phenyl)-4,5- dihydro-1H-1,4- methanobenzo[d] azepine-3(2H)- carboxamide | ¹H NMR (400 MHz, CDCl₃): δ 8.67 (d, J = 4.4 Hz, 1H), 7.72 (td, J = 2.0, 8.0 Hz, 1H), 7.47-7.34 (m, 3H), 7.25-7.21 (m, 1H), 7.21-7.16 (m, 2H), 7.16-7.06 (m, 3H), 6.13 (br s, 1H), 4.63 (br s, 1H), 3.68-3.65 (m, 1H), | m/z = 370.2 [M + H]⁺ |
| | mixture of enantiomers | | 3.45-3.42 (m, J = 8.0 | |
| | | | Hz, 1H), 3.37-3.36 (m, | |
| | | | 1H), 3.29-2.98 (m, 2H), | |
| | | | 2.31 (s, 3H), 2.21-2.16 | |
| | | | (m, 1H), 2.01-1.98 (m, | |
| | | | 1H) | |
| 22 | 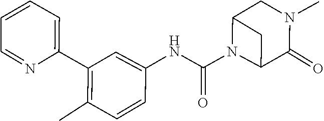 [SMILES: Cc1ccc(NC(=O)N2C3CC2C(=O)N(C)C3)cc1-c1ccccn1] | 3-methyl-N-(4- methyl-3- (pyridin-2- yl)phenyl)-2-oxo- 3,6- diazabicyclo[3.1.1] heptane-6- carboxamide | ¹H NMR (400 MHz, CDCl₃): δ 8.68 (d, J = 4.4 Hz, 1H), 7.75-7.71 (m, 1H), 7.40-7.33 (m, 3H), 7.26-7.21 (m, 1H), 7.19-7.17 (m, 1H), 6.75 (s, 1H), 4.55-4.49 (m, 1H), 4.28-4.26 (m, 1H), 4.13-4.10 (m, 1H), 3.26 | m/z = 337.2 [M + H]⁺ |
| | mixture of enantiomers | | (d, J = 11.6 Hz, 1H), | |
| | | | 2.91 (s, 3H), 2.83-2.78 | |
| | | | (m, 1H), 2.30 (s, 3H), | |
| | | | 1.73 (d, J = 8.8 Hz, 1H) | |
| 23 | 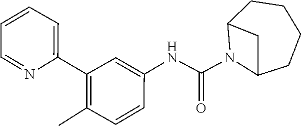 [SMILES: Cc1ccc(NC(=O)N2C3CCCCC2C3)cc1-c1ccccn1] | N-(4-methyl-3- (pyridin-2- yl)phenyl)-7- azabicyclo[4.1.1] octane-7- carboxamide | ¹H NMR (400 MHz, CDCl₃): δ 8.67 (d, J = 4.8 Hz, 1H), 7.75 (td, J = 1.6, 7.6 Hz, 1H), 7.50-7.48 (m, 1H), 7.45 (d, J = 7.6 Hz, 1H), 7.37 (d, J = 2.4 Hz, 1H), 7.26-7.23 (m, 1H), 7.20 | m/z = 322.2 [M + H]⁺ |
| | | | (d, J = 8.3 Hz, 1H), 5.83 | |
| | | | (s, 1H), 4.42 (br d, J = | |
| | | | 7.4 Hz, 2H), 2.71-2.53 | |
| | | | (m, 1H), 2.32 (s, 3H), | |
| | | | 2.25-2.14 (m, 2H), | |
| | | | 1.82-1.70 (m, 4H), | |
| | | | 1.68-1.58 (m, 3H) | |
| 24 | 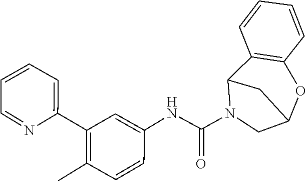 [SMILES: Cc1ccc(NC(=O)N2CC3CC2c2ccccc2O3)cc1-c1ccccn1] | N-(4-methyl-3- (pyridin-2- yl)phenyl)-2,3- dihydro-2,5- methanobenzo[f] [1,4]oxazepine- 4(5H)- carboxamide | ¹H NMR (400 MHz, CDCl₃): δ 8.67 (d, J = 4.8 Hz, 1H), 7.72 (td, J = 2.0, 7.6 Hz, 1H), 7.44-7.36 (m, 2H), 7.33 (d, J = 2.4 Hz, 1H), 7.26-7.13 (m, 4H), 6.86-6.76 (m, 2H), 6.15 (s, 1H), 5.06 (br s, 2H), 3.82-3.68 (m, 2H), 2.33-2.26 (m, 4H), 2.25-2.17 (m, 1H) | m/z = 372.2 [M + H]⁺ |
| | mixture of enantiomers | | | |
| 25 | 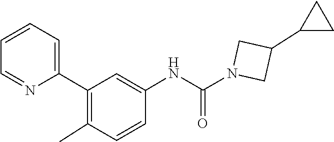 [SMILES: Cc1ccc(NC(=O)N2CC(C3CC3)C2)cc1-c1ccccn1] | 3-cyclopropyl-N- (4-methyl-3- (pyridin-2- yl)phenyl)azetidine- 1-carboxamide | ¹H NMR (400 MHz, CDCl₃): δ 8.67 (br d, J = 4.8 Hz, 1H), 7.73 (td, J = 1.6, 7.6 Hz, 1H), 7.43-7.35 (m, 1H), 7.43 (s, 2H), 7.24-7.23 (m, 1H), 7.20-7.17 (m, 1H), 6.10 (s, 1H), 4.06 (t, J = 8.0 Hz, 2H), 3.72 (t, J = | m/z = 308.2 [M + H]⁺ |
| | | | 8.0 Hz, 2H), 2.31 (s, | |
| | | | 3H), 2.27-2.17 (m, 1H), | |
| | | | 1.05-0.96 (m, 1H), | |
| | | | 0.55-0.48 (m, 2H), | |
| | | | 0.19-0.12 (m, 2H) | |
| 26 | 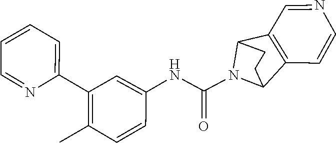 [SMILES: Cc1ccc(NC(=O)N2C3CCC2c2cnccc23)cc1-c1ccccn1] | N-(4-methyl-3- (pyridin-2- yl)phenyl)- 5,6,7,8- tetrahydro-5,8- epiminoisoquinoline- 9-carboxamide | ¹H NMR (400 MHz, CDCl₃): δ 8.71-8.63 (m, 1H), 8.52 (s, 1H), 8.47 (d, J = 4.8 Hz, 1H), 7.74 (td, J = 1.6, 7.6 Hz, 1H), 7.41 (d, J = 8.0 Hz, 1H), 7.38-7.33 (m, 2H), 7.27-7.22 (m, 2H), 7.18 (d, J = 9.2 Hz, 1H), 6.73 (s, 1H), 5.37-5.14 (m, | m/z = 357.2 [M + H]⁺ |
| | mixture of enantiomers | | 2H), 2.30 (s, 3H), 2.29- | |
| | | | 2.26 (m, 2H), 1.44-1.30 | |
| | | | (m, 2H) | |
| 27 | 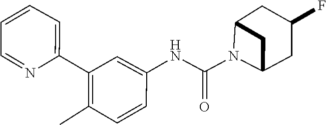 | cis-3-fluoro-N-(4- methyl-3- (pyridin-2- yl)phenyl)-6- azabicyclo[3.1.1] heptane-6- carboxamide | ¹H NMR (400 MHz, CDCl₃): δ 8.67 (d, J = 4.4 Hz, 1H), 7.76 (td, J = 1.6, 7.6 Hz, 1H), 7.47-7.42 (m, 2H), 7.39 (d, J = 2.0 Hz, 1H), 7.28-7.25 (m, 1H), 7.21 (d, J = 8.4 Hz, 1H), 6.20 | m/z = 326.2 [M + H]⁺ |
| | | | (s, 1H), 5.04 (dt, J = | |
| | | | 49.2, 6.0 Hz, 1H), 4.22 | |
| | | | (br d, J = 6.0 Hz, 2H), | |
| | | | 2.88-2.68 (m, 2H), | |
| | | | 2.56-2.51 (m, 1H), 2.32 | |
| | | | (s, 3H), 2.14-2.00 (m, | |
| | | | 2H), 1.95 (dd, J = 2.0, | |
| | | | 8.8 Hz, 1H) | |
| 28 | 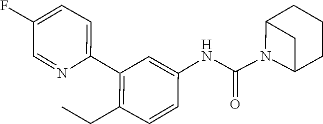 [SMILES: CCc1ccc(NC(=O)N2C3CCCC2C3)cc1-c1ccc(F)cn1] | N-(4-ethyl-3-(5- fluoropyridin-2- yl)phenyl)-6- azabicyclo[3.1.1] heptane-6- carboxamide | ¹H NMR (400 MHz, CHCl₃): δ 8.51 (d, J = 1.2 Hz, 1H), 8.45 (d, J = 4.8 Hz, 1H), 7.43-7.39 (m, 1H), 7.32 (d, J = 2.0 Hz, 1H), 7.28-7.26 (m, 1H), 7.25-7.21 (m, 1H), 6.02 (s, 1H), 4.23 (d, J = | m/z = 340.2 [M + H]⁺ |
| | | | 6.0 Hz, 2H), 2.59- | |
| | | | 2.50 (m, 1H), 2.42 (q, J = | |
| | | | 7.6 Hz, 2H), 2.41- | |
| | | | 2.32 (m, 2H), 2.03-1.89 | |
| | | | (m, 1H), 1.82-1.69 (m, | |
| | | | 3H), 1.51 (d, J = 8.8 Hz, | |
| | | | 1H), 1.07 (t, J = 8.0 Hz, | |
| | | | 3H) | |
| 29 | 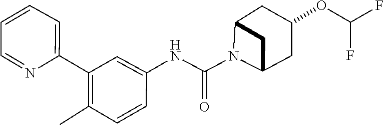 | trans-3- (difluoromethoxy)- N-(4-methyl-3- (pyridin-2- yl)phenyl)-6- azabicyclo[3.1.1] heptane-6- carboxamide | ¹H NMR (400 MHz, CDCl₃): δ 8.67 (d, J = 4.4 Hz, 1H), 7.77 (td, J = 1.6, 7.6 Hz, 1H), 7.52-7.43 (m, 1H), 7.45 (d, J = 8.0 Hz, 1H), 7.41 (d, J = 2.0 Hz, 1H), 7.29-7.25 (m, 1H), 7.21 | m/z = 374.2 [M + H]⁺ |
| | | | (d, J = 8.0 Hz, 1H), 6.23 | |
| | | | (s, 1H), 6.21 (t, J = 74 | |
| | | | Hz, 1H ), 4.93-4.85 (m, | |
| | | | 1H), 4.22 (br d, J = 5.6 | |
| | | | Hz, 2H), 2.48-2.40 (m, | |
| | | | 3H), 2.39-2.28 (m, 5H), | |
| | | | 1.49 (d, J = 8.8 Hz, 1H) | |
| 30 | 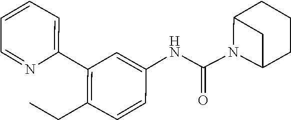 [SMILES: CCc1ccc(NC(=O)N2C3CCCC2C3)cc1-c1ccccn1] | N-(4-ethyl-3- (pyridin-2- yl)phenyl)-6- azabicyclo[3.1.1] heptane-6- carboxamide | ¹H NMR (400 MHz, CDCl₃): δ 8.68 (d, J = 4.0 Hz, 1H), 7.73 (td, J = 1.6, 7.6 Hz, 1H), 7.51 (dd, J = 2.4, 8.4 Hz, 1H), 7.42 (d, J = 7.6 Hz, 1H), 7.34 (d, J = 2.4 Hz, 1H), 7.27-7.23 (m, 2H), | m/z = 322.2 [M + H]⁺ |
| | | | 6.02 (s, 1H), 4.21 (br d, | |
| | | | J = 6.0 Hz, 2H), 2.68 (q, | |
| | | | J = 7.6 Hz, 2H), 2.58- | |
| | | | 2.50 (m, 1H), 2.44-2.34 | |
| | | | (m, 2H), 2.01-1.88 (m, | |
| | | | 1H), 1.80-1.68 (m, 3H), | |
| | | | 1.49 (d, J = 8.4 Hz, 1H), | |
| | | | 1.08 (t, J = 7.2 Hz, 3H) | |
| 31 | 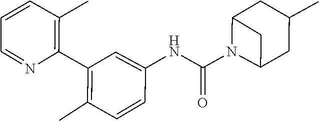 [SMILES: Cc1ccc(NC(=O)N2C3CC(C)CC2C3)cc1-c1ncccc1C] | 3-methyl-N-(4- methyl-3-(3- methylpyridin-2- yl)phenyl)-6- azabicyclo[3.1.1] heptane-6- carboxamide | ¹H NMR (400 MHz, CDCl₃): δ 8.48 (d, J = 4.4 Hz, 1H), 7.59 (d, J = 7.6 Hz, 1H), 7.45-7.40 (m, 1H), 7.22-7.17 (m, 3H), 6.10 (s, 1H), 4.20- 4.14 (m, 2H), 2.64-2.54 (m, 3H), 2.15 (s, 3H), 2.10-1.95 (m, 4H), | m/z = 336.2 [M + H]⁺. |
| | ~9:1 mixture of cis- and trans- isomers; cis signals | | 1.36-1.30 (m, 2H), | |
| | reported in NMR. | | 1.10-0.95 (m, 4H) | |
| 32 | 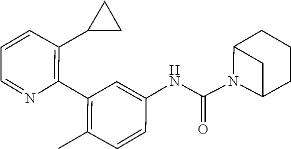 [SMILES: Cc1ccc(NC(=O)N2C3CCCC2C3)cc1-c1ncccc1C1CC1] | N-(3-(3- cyclopropylpyridin- 2-yl)-4- methylphenyl)-6- azabicyclo[3.1.1] heptane-6- carboxamide | ¹H NMR (400 MHz, CDCl₃): δ 8.44 (d, J = 6.0 Hz, 1 H), 7.53-7.49 (m, 1 H), 7.20-7.25 (m, 2 H), 7.16-7.20 (m, 2 H), 6.01 (s, 1 H), 4.20 (d, J = 6.0 Hz, 2 H), 2.55-2.51 (m, 1 H), 2.45-2.31 (m, 2 H), 2.10 | m/z = 348.3 [M + H]⁺ |
| | | | (s, 3 H), 1.89-2.00 (m, 1 | |
| | | | H), 1.76-1.68 (m, 4 H), | |
| | | | 1.49 (d, J = 8.6 Hz, 1 | |
| | | | H), 0.86-0.94 (m, 2 H), | |
| | | | 0.68-0.66 (m, 2 H) | |
| 33 | 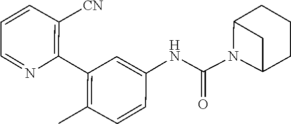 [SMILES: Cc1ccc(NC(=O)N2C3CCCC2C3)cc1-c1ncccc1C#N] | N-(3-(3- cyanopyridin-2- yl)-4- methylphenyl)-6- azabicyclo[3.1.1] heptane-6- carboxamide | ¹H NMR (400 MHz, CDCl₃): δ 8.87 (d, J = 4.8 Hz, 1H), 8.06 (dd, J = 1.2, 7.6 Hz, 1H), 7.49-7.44 (m, 2H), 7.39-7.43 (m, 1H), 7.25 (d, J = 8.4 Hz, 1H), 6.06 (s, 1H), 4.22 (br d, J = | m/z = 333.2 [M + H]⁺ |
| | | | 7.2 Hz, 2H), 2.57-2.50 | |
| | | | (m, 1H), 2.42-2.32 (m, | |
| | | | 2H), 2.21 (s, 3H), 2.02- | |
| | | | 1.89 (m, 1H), 1.81-1.68 | |
| | | | (m, 3H), 1.50 (d, J = 8.8 | |
| | | | Hz, 1H) | |
| 34 | 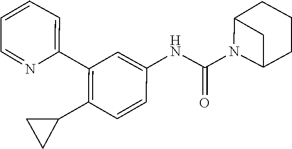 [SMILES: O=C(Nc1ccc(C2CC2)c(-c2ccccn2)c1)N1C2CCCC1C2] | N-(4-cyclopropyl- 3-(pyridin-2- yl)phenyl)-6- azabicyclo[3.1.1] heptane-6- carboxamide | ¹H NMR (400 MHz, CDCl₃): δ 8.70 (d, J = 4.8 Hz, 1H), 7.74 (dt, J = 2.0, 7.8 Hz, 1H), 7.61-7.55 (m, 1H), 7.56 (dd, J = 2.4, 8.4 Hz, 1H), 7.34 (d, J = 2.4 Hz, 1H), 7.26-7.23 (m, 1H), 6.98 (d, J = 8.4 Hz, 1H), 6.04 (s, 1H), 4.21 (br d, | m/z = 334.2 [M + H]⁺ |
| | | | J = 6.0 Hz, 2H), 2.58- | |
| | | | 2.49 (m, 1H), 2.42-2.33 | |
| | | | (m, 2H), 2.02-1.90 (m, | |
| | | | 2H), 1.77-1.69 (m, 3H), | |
| | | | 1.49 (d, J = 8.6 Hz, 1H), | |
| | | | 0.83-0.77 (m, 2H), | |
| | | | 0.65-0.59 (m, 2H) | |
| 35 | 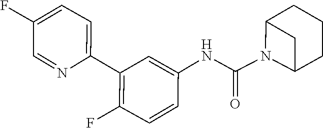 [SMILES: O=C(Nc1ccc(F)c(-c2ccc(F)cn2)c1)N1C2CCCC1C2] | N-(4-fluoro-3-(5- fluoropyridin-2- yl)phenyl)-6- azabicyclo[3.1.1] heptane-6- carboxamide | ¹H NMR (400 MHz, CDCl₃): δ 8.56 (s, 1H), 8.48 (d, J = 4.8 Hz, 1H), 7.57 (dd, J = 2.8, 6.4 Hz, 1H), 7.46-7.41 (m, 1H), 7.39 (t, J = 5.6 Hz, 1H), 7.13 (t, J = 9.2 Hz, 1H), 6.06 (s, 1H), 4.26 | m/z = 330.2 [M + H]⁺ |
| | | | (br d, J = 6.0 Hz, 2H), | |
| | | | 2.63-2.52 (m, 1H), | |
| | | | 2.43-2.31 (m, 2H), | |
| | | | 2.05-1.93 (m, 1H), | |
| | | | 1.81-1.74 (m, 3H), 1.54 | |
| | | | (d, J = 8.8 Hz, 1H) | |
| 36 | 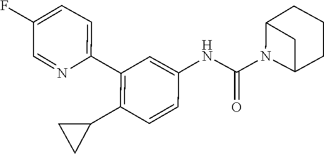 [SMILES: O=C(Nc1ccc(C2CC2)c(-c2ccc(F)cn2)c1)N1C2CCCC1C2] | N-(4-cyclopropyl- 3-(5- fluoropyridin-2- yl)phenyl)-6- azabicyclo[3.1.1] heptane-6- carboxamide | ¹H NMR (400 MHz, CDCl₃): δ 8.54 (d, J = 1.2 Hz, 1H), 8.47 (d, J = 4.8 Hz, 1H), 7.39-7.35 (m, 3H), 6.99 (d, J = 9.2 Hz, 1H), 6.03 (s, 1H), 4.23 (br d, J = 6.0 Hz, 2H), 2.58-2.52 (m, 1H), 2.41-2.33 (m, 2H), 2.01-1.91 (m, 1H), | m/z = 352.2 [M + H]⁺ |
| | | | 1.79-1.73 (m, 3H), | |
| | | | 1.74-1.65 (m, 1H), 1.51 | |
| | | | (d, J = 8.8 Hz, 1H), | |
| | | | 0.79-0.74 (m, 2H), | |
| | | | 0.59-0.55 (m, 2H) | |
| 37 | 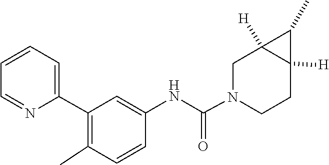 [SMILES: Cc1ccc(NC(=O)N2CC[C@@H]3[C@@H](C)[C@@H]3C2)cc1-c1ccccn1] | trans-7-methyl-N- (4-methyl-3- (pyridin-2- yl)phenyl)-3- azabicyclo[4.1.0] heptane-3- carboxamide | ¹H NMR (400 MHz, CDCl₃): δ 8.67 (d, J = 4.0 Hz, 1H), 7.72 (dt, J = 2.0, 8.0 Hz, 1H), 7.45-7.34 (m, 3H), 7.25-7.21 (m, 1H), 7.19 (d, J = 8.4 Hz, 1H), 6.32 (s, 1H), 3.70 (d, J = 3.2 Hz, 2H), 3.39-3.28 (m, 1H), 3.19-3.07 (m, 1H), 2.31 (s, 3H), 2.08-1.98 | m/z = 322.2 [M + H]⁺ |
| | mixture of enantiomers | | (m, 1H), 1.77-1.68 (m, | |
| | | | 1H), 1.03 (d, J = 6.0 Hz, | |
| | | | 3H), 0.82-0.71 (m, 2H), | |
| | | | 0.66-0.57 (m, 1H) | |
| 38 | 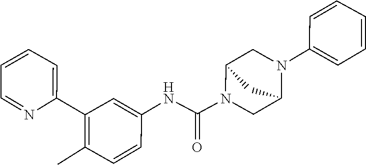 [SMILES: Cc1ccc(NC(=O)N2C[C@H]3C[C@@H]2CN3c2ccccc2)cc1-c1ccccn1] | (1R,4R)-N-(4- methyl-3- (pyridin-2- yl)phenyl)-5- phenyl-2,5- diazabicyclo[2.2.1] heptane-2- carboxamide | ¹H NMR (400 MHz, CDCl₃): δ 8.66 (d, J = 5.2 Hz, 1H), 7.72 (td, J = 1.6, 7.6 Hz, 1H), 7.43-7.32 (m, 3H), 7.26-7.21 (m, 3H), 7.17 (d, J = 8.0 Hz, 1H), 6.73 (t, J = 7.2 Hz, 1H), 6.58 (d, J = 8.0 Hz, 2H), 6.11 (s, 1H), 4.82 (s, 1H), | m/z = 385.2 [M + H]⁺ |
| | | | 4.52 (s, 1H), 3.65-3.62 | |
| | | | (m, 1H), 3.52 (s, 2H), | |
| | | | 3.32-3.29 (m, 1H), 2.30 | |
| | | | (s, 3H), 2.10-1.96 (m, | |
| | | | 2H) | |
| 39 | 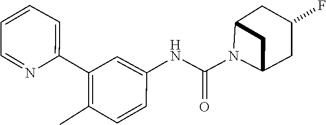 | trans-3-hydroxy- N-(4-methyl-3- (pyridin-2- yl)phenyl)-6- azabicyclo[3.1.1] heptane-6- carboxamide | ¹H NMR (400 MHz, CDCl₃): δ 8.66 (d, J = 4.4 Hz, 1H), 7.72 (td, J = 1.6, 7.6 Hz, 1H), 7.44-7.35 (m, 3H), 7.25-7.22 (m, 1H), 7.17 (d, J = 8.0 Hz, 1H), 6.45 (s, 1H), 4.39-4.32 (m, | m/z = 324.2 [M + H]⁺ |
| | | | 1H), 4.19 (br d, J = 6.0 | |
| | | | Hz, 2H), 2.39-2.32 (m, | |
| | | | 1H), 2.29 (s, 3H), 2.27- | |
| | | | 2.15 (m, 4H), 1.41 (d, J = | |
| | | | 8.4 Hz, 1H) | |
| 40 | 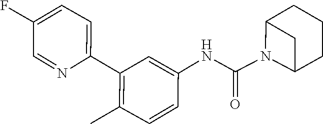 [SMILES: Cc1ccc(NC(=O)N2C3CCCC2C3)cc1-c1ccc(F)cn1] | N-(3-(5- fluoropyridin-2- yl)-4- methylphenyl)-6- azabicyclo[3.1.1] heptane-6- carboxamide | ¹H NMR (400 MHz, CDCl₃): δ 8.53 (d, J = 2.4 Hz, 1H), 7.49-7.38 (m, 4H), 7.20 (d, J = 7.6 Hz, 1H), 6.06 (s, 1H), 4.22 (br d, J = 6.0 Hz, 2H), 2.53 (q, J = 6.4 Hz, 1H), 2.44-2.33 (m, 2H), | m/z = 326.2 [M + H]⁺ |
| | | | 2.30 (s, 3H), 2.02-1.87 | |
| | | | (m, 1H), 1.81-1.70 (m, | |
| | | | 3H), 1.49 (d, J = 8.4 Hz, | |
| | | | 1H) | |
| 41 | 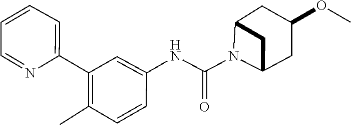 | cis-3-methoxy-N- (4-methyl-3- (pyridin-2- yl)phenyl)-6- azabicyclo[3.1.1] heptane-6- carboxamide | ¹H NMR (400 MHz, CDCl₃): δ 8.69 (d, J = 4.8 Hz, 1H), 7.74 (td, J = 1.6, 7.6 Hz, 1H), 7.49-7.41 (m, 2H), 7.38 (d, J = 2.4 Hz, 1H), 7.26-7.20 (m, 2H), 6.04 (s, 1H), 4.20 (br d, J = | m/z = 338.2 [M + H]⁺ |
| | | | 6.0 Hz, 2H), 3.75-3.67 | |
| | | | (m, 1H), 3.32 (s, 3H), | |
| | | | 2.80-2.75 (m, 2H), | |
| | | | 2.57-2.47 (m, 1H), 2.32 | |
| | | | (s, 3H), 1.81 (d, J = | |
| | | | 15.2 Hz, 2H), 1.73 (d, J = | |
| | | | 8.4 Hz, 1H) | |
| 42 | 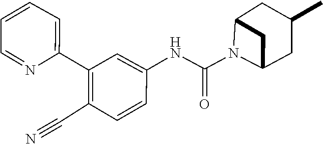 | cis-N-(4-cyano-3- (pyridin-2- yl)phenyl)-3- methyl-6- azabicyclo[3.1.1] heptane-6- carboxamide | ¹H NMR (400 MHz, CDCl₃) δ 8.74 (d, J = 4.8 Hz, 1H), 7.86-7.81 (m, 4H), 7.71-7.68 (m, 1H), 7.38-7.35 (m, 1H), 6.76 (s, 1H), 4.28-4.26 (m, 2H), 2.69-2.61 (m, 1H), 2.60-2.52 (m, 2H), 2.08-1.96 (m, 1H), | m/z = 333.2 [M + H] ⁺ |
| | | | 1.40-1.34 (m, 2H), 1.10 | |
| | | | (d, J = 8.4 Hz, 1H), 1.04 | |
| | | | (d, J = 6.8 Hz, 3H). | |
| 43 | 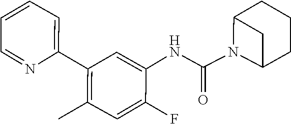 [SMILES: Cc1cc(F)c(NC(=O)N2C3CCCC2C3)cc1-c1ccccn1] | N-(2-fluoro-4- methyl-5- (pyridin-2- yl)phenyl)-6- azabicyclo[3.1.1] heptane-6- carboxamide | ¹H NMR (400 MHz, CDCl₃): δ 8.66 (d, J = 4.8 Hz, 1 H) 8.22 (d, J = 8.4 Hz, 1 H) 7.74 (t, J = 7.6 Hz, 1 H) 7.44 (d, J = 8.0 Hz, 1 H) 7.26-7.22 (m, 1 H), 6.99 (d, J = 12.0 Hz, 1 H) 6.18 (s, 1 | m/z = 326.2 [M + H]⁺ |
| | | | H), 4.28-4.22 (m, 2 H), | |
| | | | 2.64-2.51 (m, 1 H), | |
| | | | 2.46-2.35 (m, 2 H), 2.32 | |
| | | | (s, 3 H), 2.03-1.94 (m, 1 | |
| | | | H), 1.82-1.71 (m, 3 H), | |
| | | | 1.51 (d, J = 8.8 Hz, 1 H) | |
| 44 | 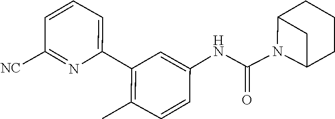 [SMILES: Cc1ccc(NC(=O)N2C3CCCC2C3)cc1-c1cccc(C#N)n1] | N-(3-(6- cyanopyridin-2- yl)-4- methylphenyl)-6- azabicyclo[3.1.1] heptane-6- carboxamide | ¹H NMR (400 MHz, CDCl₃): δ 7.88 (t, J = 8.0 Hz, 1H), 7.71-7.63 (m, 2H), 7.56 (d, J = 2.4 Hz, 1H), 7.41-7.27 (m, 1H), 7.23-7.21 (t, J = 8.0 Hz, 1H), 6.06 (s, 1H), 4.24 (br d, J = 6.0 | m/z = 333.2 [M + H]⁺ |
| | | | Hz, 2H), 2.60-2.51 (m, | |
| | | | 1H), 2.43-2.31 (m, 5H), | |
| | | | 2.03-1.92 (m, 1H), | |
| | | | 1.81-1.71 (m, 3H), 1.51 | |
| | | | (d, J = 8.4 Hz, 1H) | |
| 45 | 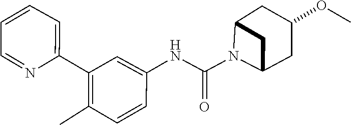 | trans-3-Methoxy- N-(4-methyl-3- (pyridin-2- yl)phenyl)-6- azabicyclo[3.1.1] heptane-6- carboxamide | ¹H NMR (400 MHz, CDCl₃): δ 8.68 (d, J = 4.4 Hz, 1H), 7.73 (td, J = 1.6, 7.6 Hz, 1H), 7.48 (dd, J = 2.4, 8.0 Hz, 1H), 7.43 (d, J = 7.6 Hz, 1H), 7.36 (d, J = 2.4 Hz, 1H), 7.27-7.22 (m, 1H), | m/z = 338.2 [M + H]⁺ |
| | | | 7.20 (d, J = 8.4 Hz, 1H), | |
| | | | 6.06 (s, 1H), 4.22 (d, J = | |
| | | | 6.0 Hz, 2H), 4.00- | |
| | | | 3.92 (m, 1H), 3.29 (s, | |
| | | | 3H), 2.43-2.35 (m, 1H), | |
| | | | 2.32 (s, 3H), 2.30-2.20 | |
| | | | (m, 4H), 1.48 (d, J = 8.8 | |
| | | | Hz, 1H) | |
| 46 | 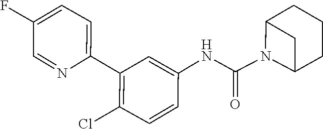 [SMILES: O=C(Nc1ccc(Cl)c(-c2ccc(F)cn2)c1)N1C2CCCC1C2] | N-(4-chloro-3-(5- fluoropyridin-2- yl)phenyl)-6- azabicyclo[3.1.1] heptane-6- carboxamide | ¹H NMR (400 MHz, CDCl₃): δ 8.55 (d, J = 2.8 Hz, 1H), 7.72 (dd, J = 4.8, 8.8 Hz, 1H), 7.73-7.70 (m, 1H), 7.64-7.61 (m, 1H), 7.48 (td, J = 5.2, 8.4 Hz, 1H), 7.39 (d, J = 8.8 Hz, | m/z = 346.1 [M + H]⁺ |
| | | | 1H), 6.09 (s, 1H), 4.24 | |
| | | | (br d, J = 6.4 Hz, 2H), | |
| | | | 2.59-2.52 (m, 1H), | |
| | | | 2.40-2.33 (m, 2H), | |
| | | | 2.02-1.91 (m, 1H), | |
| | | | 1.79-1.72 (m, 3H), 1.52 | |
| | | | (d, J = 8.8 Hz, 1H) | |

### Examples 47, 48, and 49

### cis-2-methyl-N-(4-methyl-3-(pyridin-2-yl)phenyl)-6-azabicyclo[3.1.1]heptane-6-carboxamide (47) and (1R,2S,5R)-2-methyl-N-(4-methyl-3-(pyridin-2-yl)phenyl)-6-azabicyclo[3.1.1]heptane-6-carboxamide and (1S,2R,53)-2-methyl-N-(4-methyl-3-(pyridin-2-yl)phenyl)-6-azabicyclo[3.1.1]heptane-6-carboxamide (48 and 49)

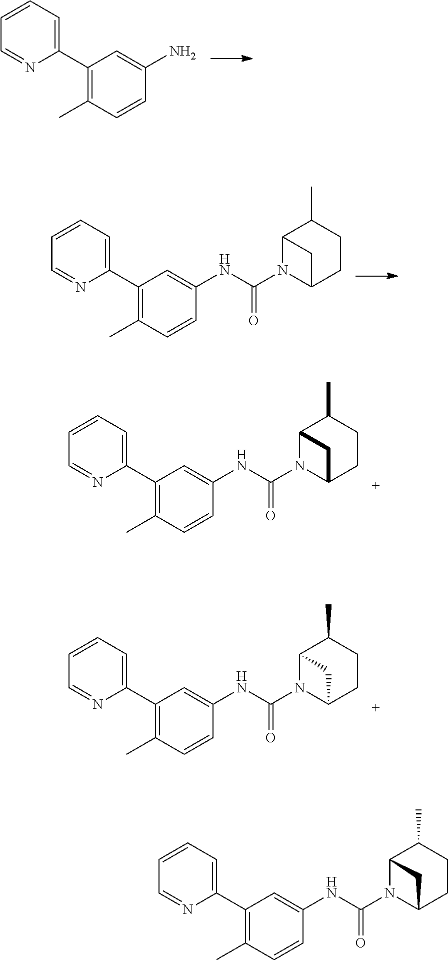

2-methyl-N-(4-methyl-3-(pyridin-2-yl)phenyl)-6-azabicyclo[3.1.1]heptane-6-carboxamide: To a mixture of CDI (52 mg, 0.32 mmol) in DCM (2 mL) was added dropwise a solution of 4-methyl-3-(2-pyridyl)aniline (50 mg, 0.27 mmol) in DCM (2 mL) over 10 min at −20° C. under N₂. The mixture was stirred at −20° C. for 2 h. Then a mixture of 2-methyl-6-azabicyclo[3.1.1]heptane (20 mg, 0.18 mmol) and TEA (54 mg, 0.54 mmol) in CH₂Cl₂ (2 mL) was added at 25° C. The mixture was stirred at 50° C. for 0.5 h and then concentrated under reduced pressure. The resulting residue was purified by prep-HPLC (column: Waters Xbridge BEH C18 100×30 mm×10 m; mobile phase: A: NH₄HCO₃ in water, B: MeCN; B % in A: 30%-60% eluting over 10 min) to give the titled compound. ¹H NMR (400 MHz, CDCl₃): δ 8.68 (d, J=4.4 Hz, 1H), 7.75-7.71 (m, 1H), 7.52-7.40 (m, 2H), 7.36 (d, J=2.4 Hz, 1H), 7.26-7.18 (m, 2H), 6.00 (s, 1H), 4.33-4.23 (m, 1H), 4.13-4.10 (m, 1H), 2.75-2.66 (m, 1H), 2.41-2.26 (m, 4H), 2.13-1.97 (m, 2H), 1.79-1.68 (m, 1H), 1.54-1.45 (m, 1H), 1.28 (d, J=8.4 Hz, 1H), 1.18 (d, J=6.8 Hz, 3H). LCMS: m/z=322.0 [M+H]⁺.

cis-2-methyl-N-(4-methyl-3-(pyridin-2-yl)phenyl)-6-azabicyclo[3.1.1]heptane-6-carboxamide, (1R,2S,5R)-2-methyl-N-(4-methyl-3-(pyridin-2-yl)phenyl)-6-azabicyclo[3.1.1]heptane-6-carboxamide and (1S,2R,5S)-2-methyl-N-(4-methyl-3-(pyridin-2-yl)phenyl)-6-azabicyclo[3.1.1]heptane-6-carboxamide: 2-methyl-N-(4-methyl-3-(pyridin-2-yl)phenyl)-6-azabicyclo[3.1.1]heptane-6-carboxamide was separated by SFC (Instrument: pre-SFC-2; Column: DAICEL CHIRALPAK AD (250 mm×30 mm, 10 μm); Mobile phase: 0.1% NH₃H₂O IPA; Gradient: B %=5% isocratic elution mode; Flow rate: 3.4 mL/min; Wavelength: 220 nm; Column temperature: 35° C.; System back pressure: 1800 psi, 3 min) to give:

cis-2-methyl-N-(4-methyl-3-(pyridin-2-yl)phenyl)-6-azabicyclo[3.1.1]heptane-6-carboxamide (47) as the first eluting peak isolated as a mixture of enantiomers. ¹H NMR (400 MHz, CDCl₃): δ 8.68 (d, J=4.4 Hz, 1H), 7.78-7.70 (m, 1H), 7.49-7.42 (m, 2H), 7.38 (d, J=2.0 Hz, 1H), 7.26-7.19 (m, 2H), 6.02 (s, 1H), 4.21 (br d, J=2.8 Hz, 1H), 3.92 (br d, J=3.2 Hz, 1H), 2.56-2.50 (m, 1H), 2.38-2.34 (m, 1H), 2.32 (s, 3H), 1.91-1.84 (m, 1H), 1.60 (d, J=8.8 Hz, 1H), 1.55-1.44 (m, 1H), 1.21 (d, J=6.4 Hz, 2H), 0.91 (d, J=6.8 Hz, 3H). LCMS: m/z=322.2 [M+H]⁺.

trans-2-methyl-N-(4-methyl-3-(pyridin-2-yl)phenyl)-6-azabicyclo[3.1.1]heptane-6-carboxamide (48) as the second eluting peak. ¹H NMR (400 MHz, CDCl₃): δ 8.67 (d, J=4.4 Hz, 1H), 7.74-7.70 (m, 1H), 7.46-7.41 (m, 2H), 7.37 (d, J=2.4 Hz, 1H), 7.24-7.17 (m, 2H), 6.13 (s, 1H), 4.27-4.24 (m, 1H), 4.13-4.09 (m, 1H), 2.70-2.64 (m, 1H), 2.30 (s, 4H), 2.09-2.01 (m, 2H), 1.75-1.68 (m, 1H), 1.53-1.45 (m, 1H), 1.27-1.25 (m, 1H), 1.16 (d, J=6.8 Hz, 3H). LCMS: m/z=322.2 [M+H]⁺.

trans-2-methyl-N-(4-methyl-3-(pyridin-2-yl)phenyl)-6-azabicyclo[3.1.1]heptane-6-carboxamide (49) as the third eluting peak. ¹H NMR (400 MHz, CDCl₃): δ 8.66 (d, J=4.4 Hz, 1H), 7.73-7.69 (m, 1H), 7.44-7.40 (m, 2H), 7.37 (d, J=2.4 Hz, 1H), 7.24-7.16 (m, 2H), 6.19 (s, 1H), 4.26-4.23 (m, 1H), 4.11-4.08 (m, 1H), 2.68-2.63 (m, 1H), 2.3 (s, 4H), 2.06-2.01 (m, 2H), 1.74-1.67 (m, 1H), 1.52-1.42 (m, 1H), 1.17 (d, J=8.4 Hz, 1H), 1.15 (d, J=6.8 Hz, 3H). LCMS: m/z=322.2 [M+H]⁺.

### Example 50

### trans-N-(3-(5-fluoropyrimidin-2-yl)-4-methylphenyl)-3-methyl-6-azabicyclo[3.1.1]heptane-6-carboxamide

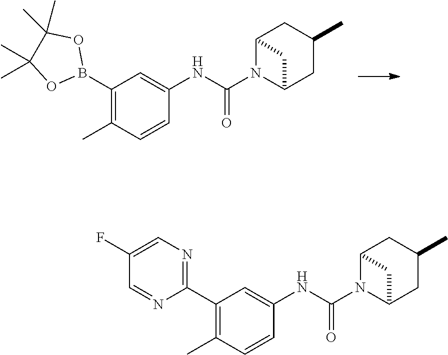

To a solution of trans-3-methyl-N-(4-methyl-3-(4,4,5,5-tetramethyl-1,3,2-dioxaborolan-2-yl)phenyl)-6-azabicyclo[3.1.1]heptane-6-carboxamide (30 mg, 0.81 mmol) in 1,4-dioxane (1 mL) and H₂O (0.2 mL) was added 2-bromo-5-fluoropyrimidine (20 mg, 0.112 mmol), K₂CO₃ (33 mg, 0.2 mmol) and Pd(dppf)Cl₂ (5 mg, 0.08 mmol) at 20° C. under N₂. The mixture was stirred at 100° C. for 12 h. The reaction mixture was concentrated under reduced pressure and the resulting residue was purified by prep-HPLC (Phenomenex C 18 100×30 mm×10 μm; mobile phase A: 10 mM NH₄HCO₃ in water, B: MeCN; B % in A: B %: 20%-50% over 8 min) to give the titled compound. ¹H NMR (400 MHz, CDCl₃): δ 8.69 (s, 2H), 7.72 (d, J=2.4 Hz, 1H), 7.63 (dd, J=2.4, 8.4 Hz, 1H), 7.23 (d, J=8.4 Hz, 1H), 6.00 (s, 1H), 4.21 (d, J=6.0 Hz, 2H), 2.49 (s, 3H), 2.41-2.38 (m, 1H), 2.34-2.25 (m, 1H), 1.95 (br d, J=8.4 Hz, 4H), 1.69 (d, J=8.4 Hz, 1H), 0.99 (d, J=6.4 Hz, 3H). LCMS: m/z=341.2 [M+H]⁺.

### Example 51

### cis-N-(3-(5-fluoropyrimidin-2-yl)-4-methylphenyl)-3-methyl-6-azabicyclo[3.1.1]heptane-6-carboxamide

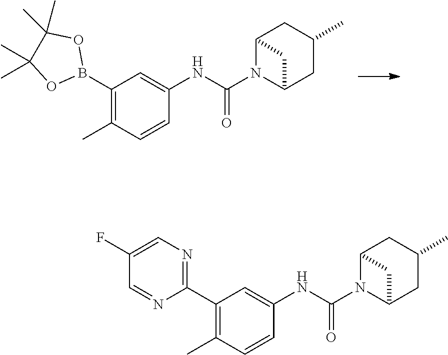

To a mixture of cis-3-methyl-N-(4-methyl-3-(4,4,5,5-tetramethyl-1,3,2-dioxaborolan-2-yl)phenyl)-6-azabicyclo[3.1.1]heptane-6-carboxamide (40 mg, 0.11 mmol) and 2-bromo-5-fluoropyrimidine (23 mg, 0.13 mmol) in 1,4-dioxane (2 mL) and H₂O (0.2 mL) was added K₂CO₃ (45 mg, 0.32 mmol) and Pd(dppf)Cl₂ (8 mg, 0.01 mmol) at 20° C. under N₂. The mixture was stirred at 100° C. for 16 h. The reaction mixture was diluted with H₂O (2 mL) and extracted with EtOAc (3×5 mL). The combined organic layers were concentrated under reduced pressure and the resulting residue was purified by prep-HPLC (Waters Xbridge BEH C18 100×30 mm×10 m; mobile phase: A: 10 mM NH₄HCO₃ in water, B: MeCN; B % in A: 35%-65% over 8 min) to give the titled compound. ¹H NMR (400 MHz, CDCl₃): δ 8.69 (s, 2H), 7.73 (d, J=2.4 Hz, 1H), 7.62 (dd, J=2.4, 8.4 Hz, 1H), 7.23 (d, J=8.4 Hz, 1H), 6.11 (s, 1H), 4.22-4.19 (m, 2H), 2.67-2.54 (m, 3H), 2.49 (s, 3H), 2.15-2.01 (m, 1H), 1.38-1.31 (m, 2H), 1.08 (d, J=8.4 Hz, 1H), 1.04 (d, J=6.4 Hz, 3H). LCMS: m/z=341.2 [M+H]⁺.

### Example 52

### N-(4-methyl-3-(pyrimidin-2-yl)phenyl)-6-azabicyclo[3.1.1]heptane-6-carboxamide

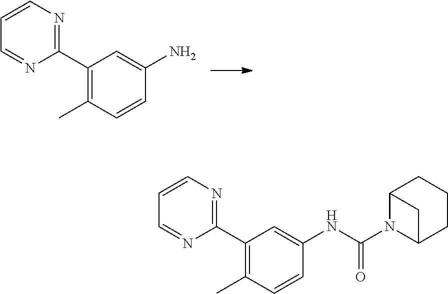 [SMILES: Cc1ccc(N)cc1-c1ncccn1 ; Cc1ccc(NC(=O)N2C3CCCC2C3)cc1-c1ncccn1]

To a solution of CDI (52 mg, 0.32 mmol) in DCM (2 mL) was added dropwise a solution of 4-methyl-3-pyrimidin-2-yl-aniline (50 mg, 0.27 mmol) in DCM (0.5 mL) over 5 min under N₂ at −20° C. The mixture was stirred at 20° C. for 12 h before adding a mixture of 6-azabicyclo[3.1.1]heptane (35 mg, 0.36 mmol) and TEA (54 mg, 0.54 mmol) in THF (2 mL). The mixture was stirred at 25° C. for an additional 12 h and then concentrated under reduced pressure. The resulting residue was purified by prep-HPLC (Phenomenex C 18 100×30 mm×3 m; mobile phase A: 10 mM NH₄HCO₃ in water, B: MeCN; B % in A: 20%-50% over 10 min) to give the titled compound. ¹H NMR (400 MHz, CDCl₃) δ 8.85 (d, J=5.2 Hz, 2H), 7.71-7.65 (m, 2H), 7.26-7.20 (m, 2H), 6.12 (s, 1H), 4.22 (d, J=6.4 Hz, 2H), 2.58-2.53 (m, 1H), 2.52 (s, 3H), 2.45-2.34 (m, 2H), 2.02-1.89 (m, 1H), 1.81-1.72 (m, 3H), 1.50 (d, J=8.4 Hz, 1H). LCMS=309.2 [M+H]⁺.

### Example 53

### N-(4-methyl-3-(pyrazin-2-yl)phenyl)-6-azabicyclo[3.1.1]heptane-6-carboxamide

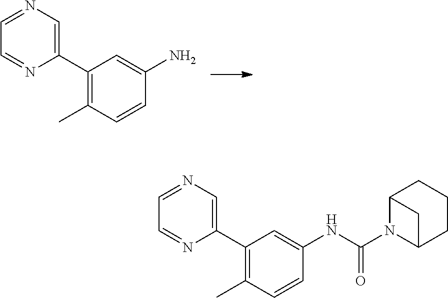 [SMILES: Cc1ccc(N)cc1-c1cnccn1 ; Cc1ccc(NC(=O)N2C3CCCC2C3)cc1-c1cnccn1]

To a solution of CDI (52 mg, 0.32 mmol) in DCM (2 mL) was added dropwise a solution of 4-methyl-3-pyrazin-2-yl-aniline (50 mg, 0.27 mmol) in DCM (0.5 mL) over 5 min at −20° C. under N₂. The mixture was stirred at 20° C. for 12 h before adding a mixture of 6-azabicyclo[3.1.1]heptane (42 mg, 0.43 mmol) and TEA (65 mg, 0.64 mmol) in DCM (2 mL). The mixture was stirred at 25° C. for 4 h and concentrated under reduced pressure. The resulting residue was purified by prep-HPLC (Phenomenex C 18 100×30 mm×3 m; mobile phase A: 10 mM NH₄HCO₃ in water, B: MeCN; B % in A: 15%-45%, over 10 min) to give the titled compound. ¹H NMR (400 MHz, CDCl₃): δ 8.75 (d, J=1.2 Hz, 1H), 8.67-8.64 (m, 1H), 8.53 (d, J=2.4 Hz, 1H), 7.52-7.46 (m, 2H), 7.25 (d, J=8.4 Hz, 1H), 6.12 (s, 1H), 4.24 (d, J=6.0 Hz, 2H), 2.58-2.56 (m, 1H), 2.46-2.37 (m, 2H), 2.35 (s, 3H), 2.02-1.92 (m, 1H), 1.82-1.72 (m, 3H), 1.52 (d, J=8.4 Hz, 1H). LCMS=309.2 [M+H]⁺.

### Example 54

### N-(4-methyl-3-(pyridazin-3-yl)phenyl)-6-azabicyclo[3.1.1]heptane-6-carboxamide

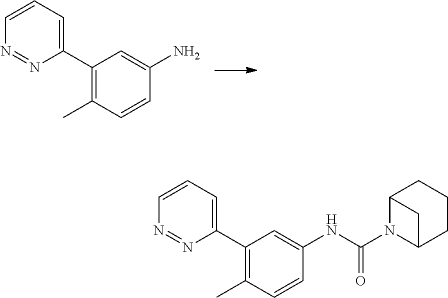 [SMILES: Cc1ccc(N)cc1-c1cccnn1 ; Cc1ccc(NC(=O)N2C3CCCC2C3)cc1-c1cccnn1]

To a solution of CDI (52 mg, 0.32 mmol) in DCM (2 mL) was added dropwise a solution of 4-methyl-3-pyridazin-3-yl-aniline (50 mg, 0.27 mmol) in DCM (2 mL) over 5 min at −20° C. under N₂. The mixture was stirred at 25° C. for 12 h before adding a mixture of 6-azabicyclo[3.1.1]heptane (21 mg, 0.21 mmol) and TEA (36 mg, 0.36 mmol) in THF (2 mL) at 20° C. The mixture was stirred at 40° C. for 3 h. The reaction mixture was concentrated under reduced pressure and the resulting residue was purified by prep-HPLC (Waters Xbridge Prep OBD C18 150×40 mm×10 m; mobile phase: A: 10 mM NH₄HCO₃ in water, B: MeCN; B % in A: 15%-45%, over 8 min) to give the titled compound. ¹H NMR (400 MHz, CDCl₃): δ 9.18 (dd, J=2.0, 5.2 Hz, 1H), 7.62-7.64 (m, 1H), 7.56-7.51 (m, 2H), 7.47 (dd, J=2.4, 8.4 Hz, 1H), 7.25 (d, J=8.4 Hz, 1H), 6.14 (s, 1H), 4.24 (br d, J=6.0 Hz, 2H), 2.59-2.50 (m, 1H), 2.43-2.36 (m, 2H), 2.34 (s, 3H), 2.01-1.91 (m, 1H), 1.79-1.71 (m, 3H), 1.50 (d, J=8.4 Hz, 1H). LCMS: m/z=309.2 [M+H]⁺.

### Example 55

### N-(3-(5-fluoropyrimidin-2-yl)-4-methylphenyl)-6-azabicyclo[3.1.1]heptane-6-carboxamide

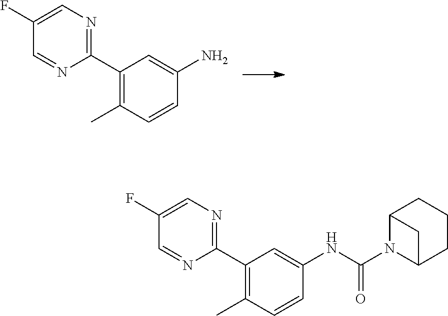 [SMILES: Cc1ccc(N)cc1-c1ncc(F)cn1 ; Cc1ccc(NC(=O)N2C3CCCC2C3)cc1-c1ncc(F)cn1]

To a mixture of triphosgene (37 mg, 0.12 mmol) in THF (1.5 mL) was added TEA (75 mg, 0.74 mmol) and 3-(5-fluoropyrimidin-2-yl)-4-methyl-aniline (50 mg, 0.25 mmol) at 0° C. under N₂. The reaction mixture was stirred at 25° C. for 1 h before adding a mixture of 6-azabicyclo[3.1.1]heptane (26 mg, 0.26 mmol) and TEA (66 mg, 0.65 mmol) in THF (1.5 mL). The mixture was stirred at 25° C. for an additional 1 h. The reaction mixture was diluted with H₂O (2 mL) and extracted with EtOAc (3×2 mL). The combined organic layers were dried over anhydrous Na₂SO₄, filtered and concentrated under reduced pressure. The resulting residue was purified by prep-HPLC (Waters Xbridge BEH C18 100×30 mm×m; mobile phase: A: 10 mM NH₄HCO₃ in water, B: MeCN; B % in A: 20%-50% over 8 min) and further purified by prep-TLC (SiO₂, EtOAc) to give the titled compound. ¹H NMR (400 MHz, CDCl₃): δ 8.69 (s, 2H), 7.74 (d, J=2.4 Hz, 1H), 7.61 (dd, J=2.4, 8.4 Hz, 1H), 7.23 (d, J=8.4 Hz, 1H), 6.09 (s, 1H), 4.23 (d, J=6.4 Hz, 2H), 2.58-2.49 (m, 1H), 2.49 (s, 3H), 2.43-2.36 (m, 2H), 2.01-1.92 (m, 1H), 1.79-1.72 (m, 3H), 1.51 (d, J=8.4 Hz, 1H). LCMS: m/z=327.2 [M+H]⁺.

The following compounds were, or can be, made via similar procedures as those described above.

| Ex. | Structure | Name | NMR | LCMS |
|---|---|---|---|---|
| 56 | 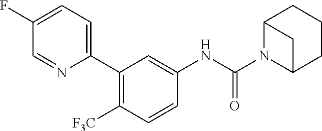 [SMILES: O=C(Nc1ccc(C(F)(F)F)c(-c2ccc(F)cn2)c1)N1C2CCCC1C2] | N-(3-(5- fluoropyridin-2-yl)- 4-(trifluoromethyl) phenyl)-6- azabicyclo[3.1.1] heptane-6-carboxamide | ¹H NMR (400 MHz, CDCl₃): δ 8.53 (s, 1H), 8.45 (d, J = 4.8 Hz, 1H), 7.72-7.66 (m, 1H), 7.65- 7.58 (m, 1H), 7.47 (s, 1H), 7.24 (t, J = 5.6 Hz, 1H), 6.29 (s, 1H), 4.27 (br d, J = 6.4 Hz, 2H), | m/z = 380.2 [M + H]⁺ |
| | | | 2.61-2.50 (m, 1H), 2.41- | |
| | | | 2.28 (m, 2H), 2.06-1.92 | |
| | | | (m, 1H), 1.85-1.69 (m, | |
| | | | 3H), 1.54 (d, J = 8.8 Hz, | |
| | | | 1H) | |
| 57 | 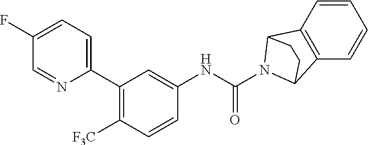 [SMILES: O=C(Nc1ccc(C(F)(F)F)c(-c2ccc(F)cn2)c1)N1C2CCC1c1ccccc12] | N-(3-(5- fluoropyridin-2-yl)- 4-(trifluoromethyl) phenyl)-1,2,3,4- tetrahydro-1,4- epiminonaphthalene- 9-carboxamide | ¹H NMR (400 MHz, CDCl₃): δ 8.51 (s, 1H), 8.43 (d, J = 4.8 Hz, 1H), 7.67-7.64 (m, 1H), 7.52- 7.50 (m, 1H), 7.41 (d, J = 2.0 Hz, 1H), 7.27-7.24 (m, 2H), 7.22-7.15 (m, 3H), 6.96 (s, 1H), 5.24- 5.19 (m, 2H), 2.23-2.19 | m/z = 428.2 [M + H]⁺ |
| | | | (m, 2H), 1.41-1.38 (m, | |
| | | | 2H) | |
| 58 | 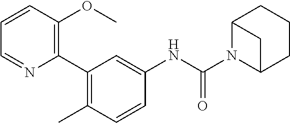 [SMILES: COc1cccnc1-c1cc(NC(=O)N2C3CCCC2C3)ccc1C] | N-(3-(3- methoxypyridin-2- yl)-4-methylphenyl)- 6-azabicyclo[3.1.1] heptane-6-carboxamide | ¹H NMR (400 MHz, CDCl₃): δ 8.28 (t, J = 3.2 Hz, 1H), 7.54 (dd, J = 2.0, 8.4 Hz, 1H), 7.26 (s, 2H), 7.23-7.16 (m, 2H), 5.99 (s, 1H), 4.19 (d, J = 6.0 Hz, 2H), 3.80 (s, 3H), 2.58-2.48 (m, 1H), 2.44- | m/z = 338.0 [M + H]⁺ |
| | | | 2.32 (m, 2H), 2.11 (s, | |
| | | | 3H), 2.00-1.87 (m, 1H), | |
| | | | 1.78-1.68 (m, 3H), 1.47 | |
| | | | (d, J = 8.4 Hz, 1H) | |
| 59 | 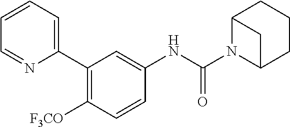 [SMILES: O=C(Nc1ccc(OC(F)(F)F)c(-c2ccccn2)c1)N1C2CCCC1C2] | N-(3-(pyridin-2-yl)- 4-(trifluoromethoxy) phenyl)-6- azabicyclo[3.1.1] heptane-6-carboxamide | ¹H NMR (400 MHz, CDCl₃): δ 8.75 (br d, J = 5.2 Hz, 1H), 7.93-7.81 (m, 2H), 7.82-7.75 (m, 1H), 7.69 (d, J = 2.0 Hz, 1H), 7.38 (t, J = 5.6 Hz, 1H), 7.33-7.29 (m, 1H), 6.33 (br s, 1H), 4.27 (d, J = | m/z = 378.2 [M + H]⁺ |
| | | | 6.0 Hz, 2H), 2.64-2.51 | |
| | | | (m, 1H), 2.44-2.33 (m, | |
| | | | 2H), 2.05-1.85 (m, 1H), | |
| | | | 1.80-1.68 (m, 3H), 1.53 | |
| | | | (d, J = 8.8 Hz, 1H) | |
| 60 | 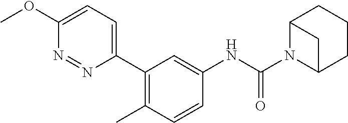 [SMILES: COc1ccc(-c2cc(NC(=O)N3C4CCCC3C4)ccc2C)nn1] | N-(3-(6- methoxypyridazin-3- yl)-4-methyl- phenyl)-6- azabicyclo[3.1.1] heptane-6-carboxamide | | m/z = 339.2 [M + H]+ |
| 61 | 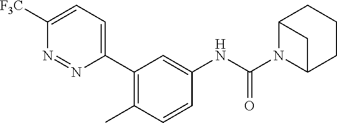 [SMILES: Cc1ccc(NC(=O)N2C3CCCC2C3)cc1-c1ccc(C(F)(F)F)nn1] | N-(4-methyl-3-(6- (trifluoromethyl) pyridazine-3-yl) phenyl)-6- azabicyclo[3.1.1] heptane-6-carboxamide | | m/z = 377.1 [M + H]⁺ |
| 62 | 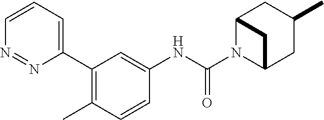 | cis-3-methyl-N-(4- methyl-3-pyridazin- 3-yl-phenyl)-6- azabicyclo[3.1.1] heptane-6-carboxamide | | m/z = 323.2 [M + H]⁺ |
| 63 | 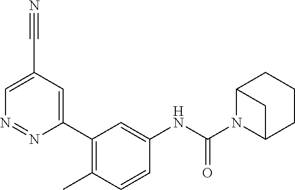 [SMILES: Cc1ccc(NC(=O)N2C3CCCC2C3)cc1-c1cc(C#N)cnn1] | N-(3-(5- cyanopyridazin-3- yl)-4-methylphenyl)- 6-azabicyclo[3.1.1] heptane-6-carboxamide | | m/z = 334.2 [M + H]⁺ |
| 64 | 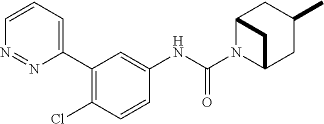 | cis-N-(4-chloro-3- pyridazin-3- ylphenyl)-3-methyl- 6-azabicyclo[3.1.1] heptane-6-carboxamide | | m/z = 343.1 [M + H]⁺ |
| 65 | 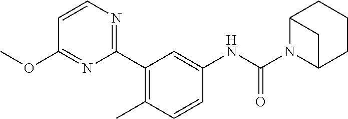 [SMILES: COc1ccnc(-c2cc(NC(=O)N3C4CCCC3C4)ccc2C)n1] | N-(3-(4- methoxypyrmidin-2- yl)-4-methyl- phenyl)-6-azabicyclo [3.1.1]heptane- 6-carboxamide | | m/z = 339.2 [M + H]⁺ |
| 66 | 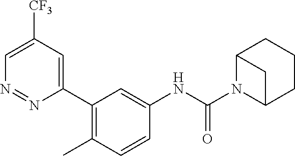 [SMILES: Cc1ccc(NC(=O)N2C3CCCC2C3)cc1-c1cc(C(F)(F)F)cnn1] | N-(4-methyl-3-(5- (trifluoromethyl) pyridazine-3-yl) phenyl)-6- azabicyclo[3.1.1] heptane-6-carboxamide | | m/z = 377.2 [M + H]⁺ |
| 67 | 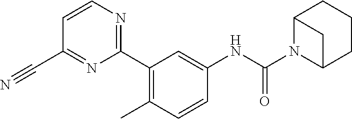 [SMILES: Cc1ccc(NC(=O)N2C3CCCC2C3)cc1-c1nccc(C#N)n1] | N-(3-(4- cyanopyrimidin-2- yl)-4-methyl- phenyl)-6- azabicyclo[3.1.1] heptane-6-carboxamide | | m/z = 334.2 [M + H]⁺ |
| 68 | 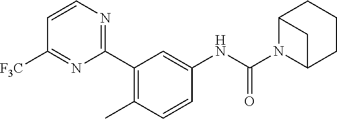 [SMILES: Cc1ccc(NC(=O)N2C3CCCC2C3)cc1-c1nccc(C(F)(F)F)n1] | N-(4-methyl-3-(4- (trifluoromethyl) pyrimidin-2-yl) phenyl)-6- azabicyclo[3.1.1] heptane-6-carboxamide | | m/z = 377.1 [M + H]⁺ |

### Example 69

### N-(3-fluoro-4-methyl-5-(pyridin-2-yl)phenyl)-6-azabicyclo[3.1.1]heptane-6-carboxamide

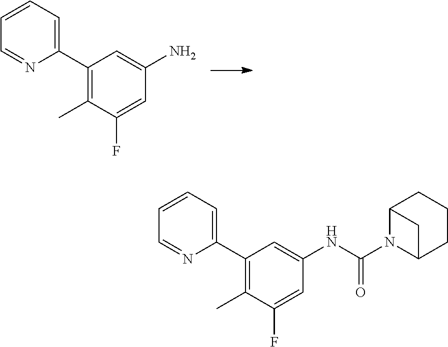 [SMILES: Cc1c(F)cc(N)cc1-c1ccccn1 ; Cc1c(F)cc(NC(=O)N2C3CCCC2C3)cc1-c1ccccn1]

To a mixture of triphosgene (37 mg, 0.12 mmol) in THF (1.5 mL) was added TEA (75 mg, 0.74 mmol) and 3-fluoro-4-methyl-5-(2-pyridyl)aniline (50 mg, 0.25 mmol) at 0° C. under N₂. The mixture was stirred at 25° C. for 1 h before adding a mixture of 6-azabicyclo[3.1.1]heptane (32 mg, 0.33 mmol) and TEA (44 mg, 0.44 mmol) in THF (1 mL). The solution was stirred for 1 h, concentrated under reduced pressure and purified by prep-HPLC (Phenomenex C18 100×30 mm×10 m; mobile phase: A: 10 mM NH₄HCO₃ in water, B: MeCN; B % in A: 25%-55% over 8 min) to give the titled compound. ¹H NMR (400 MHz, CDCl₃): δ 8.69 (d, J=4.0 Hz, 1H), 7.78-7.71 (td, J=2.4, 7.6 Hz, 1H), 7.51 (dd, J=2.0, 11.6 Hz, 1H), 7.40 (d, J=8.0 Hz, 1H), 7.27-7.25 (m, 1H), 7.09 (s, 1H), 6.15 (s, 1H), 4.25-4.21 (m, 2H), 2.57-2.49 (m, 1H), 2.41-2.30 (m, 2H), 2.20 (s, 3H), 2.00-1.89 (m, 1H), 1.76-1.70 (m, 3H), 1.50 (d, J=8.8 Hz, 1H). LCMS: m/z=326.2 [M+H]⁺.

### Example 70

### cis-methyl-6-((4-methyl-3-(pyridin-2-yl)phenyl)carbamoyl)-6-azabicyclo[3.1.1]heptane-2-carboxylate

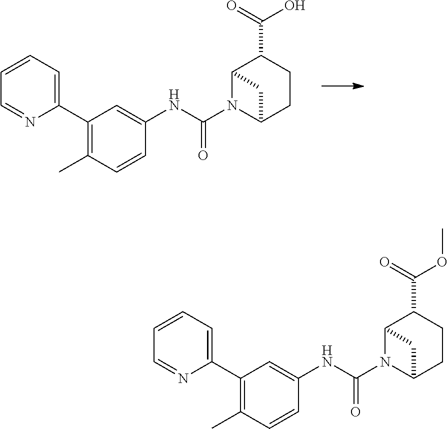

To a mixture of cis-6-((4-methyl-3-(pyridin-2-yl)phenyl)carbamoyl)-6-azabicyclo[3.1.1]heptane-2-carboxylic acid (20 mg, 0.057 mmol) in THF (1 mL) and MeOH (0.25 mL) was added dropwise TMSCHN₂ (0.14 mmol, 0.07 mL, 2 M in hexane) at 0° C. under N₂. The mixture was stirred at 25° C. for 12 h. The reaction mixture was concentrated under reduced pressure. The resulting residue was purified by prep-TLC (SiO₂, EtOAc) to give the titled compound as a mixture of enantiomers. ¹H NMR (400 MHz, CDCl₃): δ 8.67 (d, J=4.0 Hz, 1H), 7.73 (td, J=2.0, 8.0 Hz, 1H), 7.52 (d, J=2.4 Hz, 1H), 7.48 (s, 1H), 7.45-7.39 (m, 2H), 7.25-7.20 (m, 1H), 7.18 (d, J=8.4 Hz, 1H), 4.63-4.61 (m, 1H), 4.41-4.35 (m, 1H), 3.73 (s, 3H), 2.93 (t, J=8.4 Hz, 1H), 2.85-2.75 (m, 1H), 2.42-2.35 (m, 1H), 2.30 (s, 3H), 2.29-2.23 (m, 1H), 1.93-2.03 (m, 1H), 1.83-1.77 (m, 1H), 1.33 (d, J=8.8 Hz, 1H). LCMS: m/z=366.2 [M+H]⁺.

### Example 71

### N-(3,4-dimethyl-5-(pyridin-2-yl)phenyl)-6-azabicyclo[3.1.1]heptane-6-carboxamide

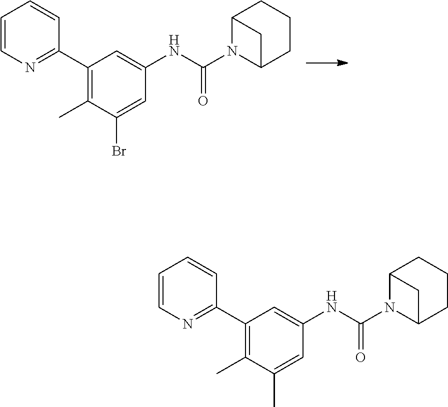 [SMILES: Cc1c(Br)cc(NC(=O)N2C3CCCC2C3)cc1-c1ccccn1 ; Cc1cc(NC(=O)N2C3CCCC2C3)cc(-c2ccccn2)c1C]

To a solution of N-(3-bromo-4-methyl-5-(pyridin-2-yl)phenyl)-6-azabicyclo[3.1.1]heptane-6-carboxamide (50 mg, 0.13 mmol) in 1,4-dioxane (2 mL) and H₂O (0.2 mL) was added 2,4,6-trimethyl-1,3,5,2,4,6-trioxatriborinane (0.18 mL, 50% purity in THF), K₂CO₃ (36 mg, 0.26 mmol) and Pd(dppf)Cl₂ (10 mg, 0.13 mmol) at 20° C. under N₂. The mixture was stirred at 100° C. for 12 h. The reaction mixture was filtered and concentrated under reduced pressure. The resulting residue was purified by prep-HPLC (column: Waters Xbridge Prep OBD C18 150×40 mm×10 m; mobile phase: A: 10 mM NH₄HCO₃ in water; B: MeCN; B % in A: 25%-55%, over 8 min) to give the titled compound. ¹H-NMR (400 MHz, CDCl₃): δ 8.69 (br d, J=4.8 Hz, 1H), 7.75 (t, J=7.6 Hz, 1H), 7.47-7.39 (m, 2H), 7.26-7.23 (m, 1H), 7.19 (d, J=2.0 Hz, 1H), 5.99 (s, 1H), 4.21 (br d, J=6.0 Hz, 2H), 2.59-2.47 (m, 1H), 2.42-2.35 (m, 2H), 2.33 (s, 3H), 2.17 (s, 3H), 1.99-1.88 (m, 1H), 1.80-1.69 (m, 3H), 1.49 (d, J=8.8 Hz, 1H). LCMS: m/z=322.2 [M+H]⁺.

### Example 72

### N-(4-methyl-3-(pyridin-2-yl)phenyl)-3-(pyrimidin-2-yl)-3,6-diazabicyclo[3.1.1]heptane-6-carboxamide

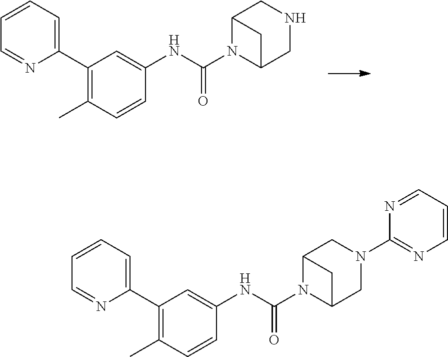 [SMILES: Cc1ccc(NC(=O)N2C3CNCC2C3)cc1-c1ccccn1 ; Cc1ccc(NC(=O)N2C3CC2CN(c2ncccn2)C3)cc1-c1ccccn1]

To a mixture of N-[4-methyl-3-(2-pyridyl)phenyl]-3,6-diazabicyclo[3.1.1]heptane-6-carboxamide (100 mg, 0.32 mmol) and 2-chloropyrimidine (74 mg, 0.66 mmol) in DMF (4 mL) was added K₂CO₃ (90 mg, 0.65 mmol) at 20° C. under N₂. The mixture was stirred at 90° C. for 12 h. The reaction mixture was concentrated under reduced pressure. The resulting residue was purified by prep-HPLC (Phenomenex C18 75×30 mm×3 m; mobile phase: A: 10 mM NH₄HCO₃ in water, B: MeCN; B % in A: 20%-50% over 8 min) to give the titled compound. ¹H NMR (400 MHz, CDCl₃): δ 8.67 (d, J=4.0 Hz, 1H), 8.34 (d, J=4.8 Hz, 2H), 7.75-7.71 (m, 1H), 7.41-7.36 (m, 3H), 7.24-7.18 (m, 2H), 6.57 (t, J=4.8 Hz, 1H), 6.10 (s, 1H), 4.41-4.39 (m, 2H), 4.25-3.71 (m, 4H), 2.78-2.73 (m, 1H), 2.30 (s, 3H), 1.62 (d, J=8.4 Hz, 1H). LCMS: m/z=387.2 [M+H]⁺.

### Example 73

### N-(4-methyl-3-(pyridin-2-yl)phenyl)bicyclo[2.2.1]heptane-7-carboxamide

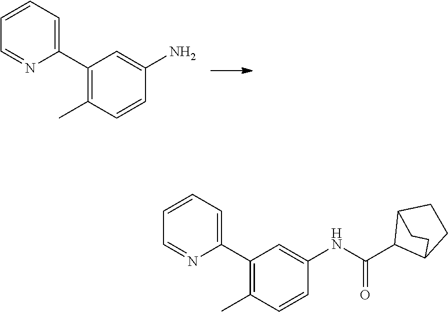 [SMILES: Cc1ccc(N)cc1-c1ccccn1 ; Cc1ccc(NC(=O)C2C3CCC2CC3)cc1-c1ccccn1]

To a solution of 4-methyl-3-(2-pyridyl)aniline (78.86 mg, 0.43 mmol), norbornane-7-carboxylic acid (50.0 mg, 0.36 mmol), and 1-[bis(dimethylamino)methylene]-1H-1,2,3-triazolo[4,5-b]pyridinium 3-oxide hexafluorophosphate (50 mg, 0.36 mmol) in DMF (1.5 mL) was added N,N′-diisopropylethylamine (0.25 mL, 1.43 mmol). The reaction mixture was stirred at room temperature for 1 h. The reaction was diluted with water, extracted with a 1:1 mixture of ethyl acetate:ether twice. The combined organics were washed with water and brine three times each. The organic layer was dried over Na₂SO₄ and concentrated. The crude residue was purified by prep HPLC to give the titled compound. ¹H-NMR (400 MHz; CDCl₃): δ 8.73 (ddd, J=5.0, 1.7, 0.8 Hz, 1H), 7.82 (td, J=7.7, 1.7 Hz, 1H), 7.58-7.55 (m, 2H), 7.50 (d, J=7.8 Hz, 1H), 7.33 (ddd, J=7.6, 5.1, 1.0 Hz, 1H), 7.28-7.24 (m, 1H), 2.55 (dd, J=1.1, 0.3 Hz, 1H), 2.52 (dd, J=1.3, 0.5 Hz, 2H), 2.35 (s, 3H), 1.90-1.87 (m, 2H), 1.71-1.68 (m, 2H), 1.34-1.31 (m, 4H). LCMS: m/z=307.2 [M+H]⁺.

The following compounds were, or can be, made via similar procedures as those described above.

| Ex. | Structure | Name | NMR | LCMS |
|---|---|---|---|---|
| 74 | 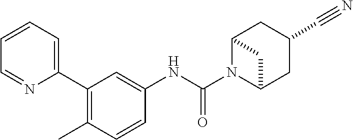 | cis-3-cyano-N-(4- methyl-3-(pyridin- 2-yl)phenyl)-6- azabicyclo[3.1.1] heptane-6- carboxamide | ¹H NMR (400 MHz, CDCl₃): δ 8.66 (d, J = 4.4 Hz, 1H), 7.78-7.71 (m, 1H), 7.46-7.34 (m, 3H), 7.28-7.26 (m, 1H), 7.21 (d, J = 8.0 Hz, 1H), 6.39 (br s, 1H), 4.28-4.19 (m, 2H), 3.10-3.02 (m, 1H), 2.82 | m/z = 333.2 [M + H]⁺ |
| | | | (t, J = 12.0 Hz, 2H), | |
| | | | 2.75-2.63 (m, 1H), 2.32 | |
| | | | (s, 3H), 2.06-2.01 (m, | |
| | | | 2H), 1.60 (d, J = 9.2 | |
| | | | Hz, 1H) | |
| 75 | 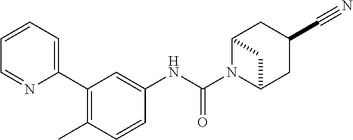 | trans-3-cyano-N- (4-methyl-3- (pyridin-2- yl)phenyl)-6- azabicyclo[3.1.1] heptane-6- carboxamide | ¹H NMR (400 MHz, CD₃CN): δ 8.65 (d, J = 4.8 Hz, 1H), 7.81 (td, J = 1.6, 7.6 Hz, 1H), 7.50 (d, J = 2.4 Hz, 1H), 7.45 (dd, J = 0.8, 7.6 Hz, 2H), 7.32-7.30 (m, 1H), 7.20 (d, J = 8.4 Hz, 1H), 7.13 (br s, | m/z = 333.2 [M + H]⁺ |
| | | | 1H), 4.22 (br d, J = 5.6 | |
| | | | Hz, 2H), 3.47-3.38 (m, | |
| | | | 1H), 2.52-2.42 (m, 3H), | |
| | | | 2.27 (s, 3H), 2.23-2.17 | |
| | | | (m, 2H), 1.53 (d, J = 9.6 | |
| | | | Hz, 1H) | |
| 76 | 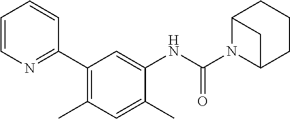 [SMILES: Cc1cc(C)c(-c2ccccn2)cc1NC(=O)N1C2CCCC1C2] | N-(2,4-dimethyl-5- (pyridin-2- yl)phenyl)-6- azabicyclo[3.1.1] heptane-6- carboxamide | ¹H NMR (400 MHz, CDCl₃): δ 8.66 (d, J = 4.8 Hz, 1H), 7.76 (s, 1H), 7.71 (td, J = 2.4, 7.6 Hz, 1H), 7.43 (d, J = 7.6 Hz, 1H), 7.23- 7.20 (m, 1H), 7.08 (s, 1H), 5.85 (s, 1H), 4.18 | m/z = 322.2 [M + H]⁺ |
| | | | (d, J = 6.4 Hz, 2H), | |
| | | | 2.55-2.53 (m, 1H), | |
| | | | 2.43-2.38 (m, 2H), 2.32 | |
| | | | (s, 3H), 2.28 (s, 3H), | |
| | | | 1.99-1.91 (m, 1H), | |
| | | | 1.80-1.70 (m, 3H), 1.48 | |
| | | | (d, J = 8.4 Hz, 1H) | |
| 77 | 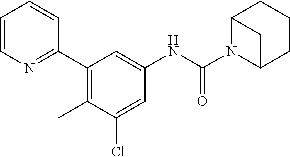 [SMILES: Cc1c(Cl)cc(NC(=O)N2C3CCCC2C3)cc1-c1ccccn1] | N-(3-chloro-4- methyl-5-(pyridin- 2-yl)phenyl)-6- azabicyclo[3.1.1] heptane-6- carboxamide | ¹H NMR (400 MHz, CDCl₃): δ 8.68 (br d, J = 4.8 Hz, 1H), 7.78- 7.72 (m, 1H), 7.70 (d, J = 2.0 Hz, 1H), 7.39 (d, J = 7.6 Hz, 1H), 7.28- 7.25 (m, 2H), 6.04 (s, 1H), 4.23-4.21 (m, 2H), 2.59-2.49 (m, 1H), 2.41-2.30 (m, 2H), 2.29 | m/z = 342.2 [M + H]⁺ |
| | | | (s, 3H), 1.96-1.94 (m, | |
| | | | 1H), 1.77-1.71 (m, 3H), | |
| | | | 1.50 (d, J = 8.4 Hz, 1H) | |
| 78 | 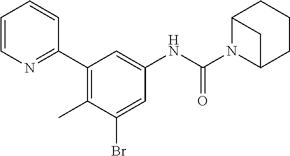 [SMILES: Cc1c(Br)cc(NC(=O)N2C3CCCC2C3)cc1-c1ccccn1] | N-(3-bromo-4- methyl-5-(pyridin- 2-yl)phenyl)-6- azabicyclo[3.1.1] heptane-6- carboxamide | ¹H NMR (400 MHz, CDCl₃): δ 8.68 (d, J = 4.8 Hz, 1H), 7.86 (d, J = 2.4 Hz, 1H), 7.75 (td, J = 1.8, 7.6 Hz, 1H), 7.38 (d, J = 8.0 Hz, 1H), 7.35 (d, J = 2.4 Hz, 1H), 7.27-7.25 (m, 1H), 6.02 (s, 1H), 4.23- 4.21 (m, 2H), 2.57-2.53 | m/z = 386.1, 388.1 [M + H]⁺ |
| | | | (m, 1H), 2.37-2.32 (m, | |
| | | | 2H), 2.32 (s, 3H), 1.96- | |
| | | | 1.94 (m, 1H), 1.77-1.71 | |
| | | | (m, 3H), 1.50 (d, J = 8.8 | |
| | | | Hz, 1H) | |
| 79 | 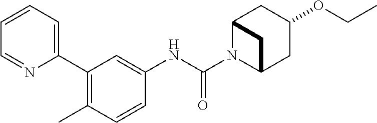 | trans-3-ethoxy-N- (4-methyl-3- (pyridin-2- yl)phenyl)-6- azabicyclo[3.1.1] heptane-6- carboxamide | ¹H NMR (400 MHz, CDCl₃): δ 8.68 (d, J = 4.8 Hz, 1H), 7.77 (td, J = 1.6, 7.6 Hz, 1H), 7.51 (dd, J = 2.4, 8.4 Hz, 1H), 7.46 (d, J = 7.6 Hz, 1H), 7.39 (d, J = 2.4 Hz, 1H), 7.27-7.25 | m/z = 352.2 [M + H]⁺ |
| | | | (m, 1H), 7.21 (d, J = 8.4 | |
| | | | Hz, 1H), 6.15 (s, 1H), | |
| | | | 4.23 (d, J = 6.0 Hz, | |
| | | | 2H), 4.12-4.02 (m, 1H), | |
| | | | 3.46 (q, J = 7.2 Hz, | |
| | | | 2H), 2.42-2.34 (m, 1H), | |
| | | | 2.32 (s, 3H), 2.30-2.21 | |
| | | | (m, 4H), 1.50 (d, J = 8.8 | |
| | | | Hz, 1H), 1.16 (t, J = 7.2 | |
| | | | Hz, 3H) | |
| 80 | 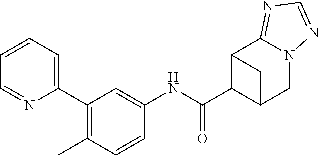 [SMILES: Cc1ccc(NC(=O)C2C3CC2c2ncnn2C3)cc1-c1ccccn1] | N-(4-methyl-3- pyridin-2- ylphenyl)-3,5,6,9- tetrazatricyclo[6.1. 1.02,6]deca-2,4- diene-9- carboxamide | | m/z = 347.1 [M + H]⁺ |
| 81 | 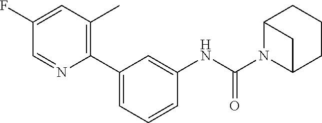 [SMILES: Cc1cc(F)cnc1-c1cccc(NC(=O)N2C3CCCC2C3)c1] | N-[3-(5-fluoro-3- methylpyridin-2- yl)phenyl]-6- azabicyclo[3.1.1] heptane-6- carboxamide | | m/z = 326.1 [M + H]⁺ |
| 82 | 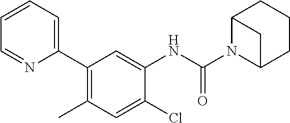 [SMILES: Cc1cc(Cl)c(NC(=O)N2C3CCCC2C3)cc1-c1ccccn1] | N-(2-chloro-4- methyl-5-pyridin-2- ylphenyl)-6- azabicyclo[3.1.1] heptane-6- carboxamide | | m/z = 342.1 [M + H]⁺ |
| 83 | 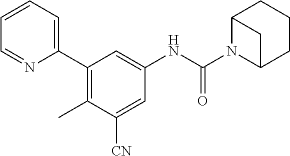 [SMILES: Cc1c(C#N)cc(NC(=O)N2C3CCCC2C3)cc1-c1ccccn1] | N-(3-cyano-4- methyl-5-pyridin-2- ylphenyl)-6- azabicyclo[3.1.1] heptane-6- carboxamide | | m/z = 333.2 [M + H]⁺ |

### Example 84

### cis-3-ethoxy-N-(4-methyl-3-(pyridin-2-yl)phenyl)-6-azabicyclo[3.1.1]heptane-6-carboxamide

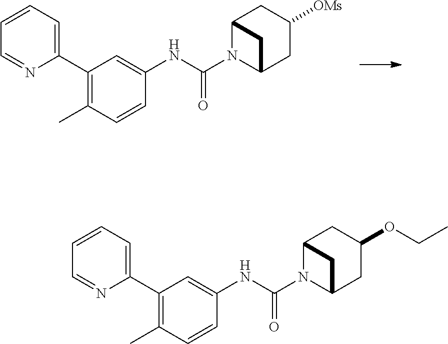

A mixture of trans-6-((4-methyl-3-(pyridin-2-yl)phenyl)carbamoyl)-6-azabicyclo[3.1.1]heptan-3-yl methanesulfonate (50 mg, 0.12 mmol) and NaOEt (34 mg, 0.50 mmol) in EtOH (1 mL) was stirred at 70° C. for 12 h under N₂. The reaction mixture was concentrated under reduced pressure, diluted with H₂O (2 mL) and extracted with EtOAc (3×3 mL). The combined organic layers were washed with brine (3 mL), dried over anhydrous Na₂SO₄, filtered and concentrated under reduced pressure. The resulting residue was purified by prep-TLC (SiO₂, PE:EtOAc=1:1) and further purified by prep-HPLC (Waters Xbridge BEH C18 100×30 mm×10 in; mobile phase: A: 10 mM NH₄HCO₃ in water; B: MeCN; B % in A: 30%-50%, over 8 min) to provide the titled compound. ¹H NMR (400 MHz, CDCl₃): δ 8.69 (d, J=4.8 Hz, 1H), 7.74 (td, J=2.0, 7.6 Hz, 1H), 7.47 (dd, J=2.4, 8.4 Hz, 1H), 7.43 (d, J=8.0 Hz, 1H), 7.38 (d, J=2.4 Hz, 1H), 7.26-7.23 (m, 1H), 7.21 (d, J=8.4 Hz, 1H), 6.04 (s, 1H), 4.20 (br d, J=6.4 Hz, 2H), 3.84-3.79 (m, 1H), 3.47 (q, J=6.8 Hz, 2H), 2.78 (br dd, J=7.6, 14.4 Hz, 2H), 2.57-2.49 (m, 1H), 2.32 (s, 3H), 1.81 (dd, J=2.8, 14.4 Hz, 2H), 1.76 (d, J=8.4 Hz, 1H), 1.19 (t, J=6.8 Hz, 3H). LCMS: m/z=352.2 [M+H]⁺.

The following compounds were, or can be, made via similar procedures as those described above. Example 98 was prepared using commercially available amine (CAS: 1311315-27-1).

| Ex. | Structure | Name | LCMS |
|---|---|---|---|
| 85 | 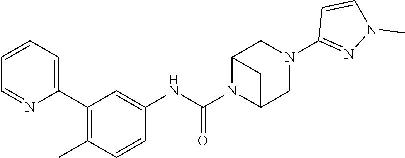 [SMILES: Cc1ccc(NC(=O)N2C3CC2CN(c2ccn(C)n2)C3)cc1-c1ccccn1] | 3-(1-methylpyrazol-3-yl)-N-(4- methyl-3-pyridin-2-ylphenyl)-3,6- diazabicyclo[3.1.1]heptane-6- carboxamide | m/z = 389.2 [M + H]⁺ |
| 86 | 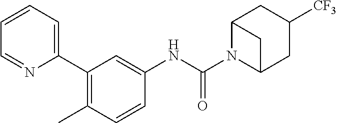 [SMILES: Cc1ccc(NC(=O)N2C3CC2CC(C(F)(F)F)C3)cc1-c1ccccn1] | N-(4-methyl-3-pyridin-2-ylphenyl)- 3-(trifluoromethyl)-6- azabicyclo[3.1.1]heptane-6- carboxamide | m/z = 376.1 [M + H]⁺ |
| | single unknown diastereomer | | |
| 87 | 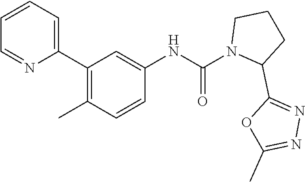 [SMILES: Cc1nnc(C2CCCN2C(=O)Nc2ccc(C)c(-c3ccccn3)c2)o1] | 2-(5-methyl-1,3,4-oxadiazol-2-yl)- N-(4-methyl-3-pyridin-2- ylphenyl)pyrrolidine-1- carboxamide | m/z = 364.2 [M + H]⁺ |
| | mixture of enantiomers | | |
| 88 | 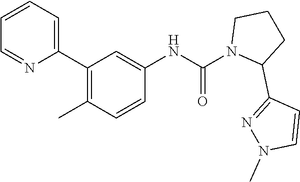 [SMILES: Cc1ccc(NC(=O)N2CCCC2c2ccn(C)n2)cc1-c1ccccn1] | 2-(1-methylpyrazol-3-yl)-N-(4- methyl-3-pyridin-2- ylphenyl)pyrrolidine-1- carboxamide | m/z = 362.2 [M + H]⁺ |
| | mixture of enantiomers | | |
| 89 | 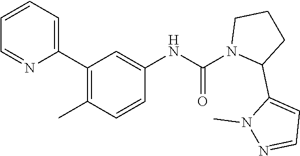 [SMILES: Cc1ccc(NC(=O)N2CCCC2c2ccnn2C)cc1-c1ccccn1] | 2-(2-methylpyrazol-3-yl)-N-(4- methyl-3-pyridin-2- ylphenyl)pyrrolidine-1- carboxamide | m/z = 362.2 [M + H]⁺ |
| | mixture of enantiomers | | |
| 90 | 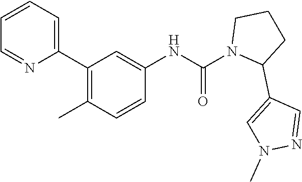 [SMILES: Cc1ccc(NC(=O)N2CCCC2c2cnn(C)c2)cc1-c1ccccn1] | 2-(1-methylpyrazol-4-yl)-N-(4- methyl-3-pyridin-2- ylphenyl)pyrrolidine-1- carboxamide | m/z = 362.2 [M + H]⁺ |
| | mixture of enantiomers | | |
| 91 | 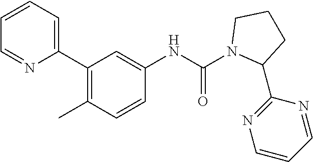 [SMILES: Cc1ccc(NC(=O)N2CCCC2c2ncccn2)cc1-c1ccccn1] | N-(4-methyl-3-pyridin-2-ylphenyl)- 2-pyrimidin-2-ylpyrrolidine-1- carboxamide | m/z = 360.2 [M + H]⁺ |
| | mixture of enantiomers | | |
| 92 | 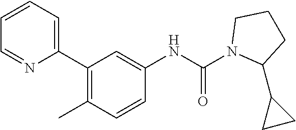 [SMILES: Cc1ccc(NC(=O)N2CCCC2C2CC2)cc1-c1ccccn1] | 2-cyclopropyl-N-(4-methyl-3- pyridin-2-ylphenyl)pyrrolidine-1- carboxamide | m/z = 322.2 [M + H]⁺ |
| | mixture of enantiomers | | |
| 93 | 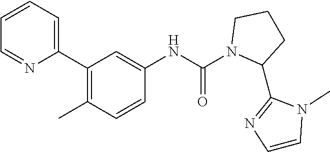 [SMILES: Cc1ccc(NC(=O)N2CCCC2c2nccn2C)cc1-c1ccccn1] | 2-(1-methylimidazol-2-yl)-N-(4- methyl-3-pyridin-2- ylphenyl)pyrrolidine-1- carboxamide | m/z = 362.2 [M + H]⁺ |
| | mixture of enantiomers | | |
| 94 | 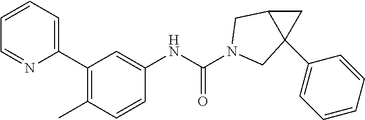 [SMILES: Cc1ccc(NC(=O)N2CC3CC3(c3ccccc3)C2)cc1-c1ccccn1] | N-(4-methyl-3-pyridin-2-ylphenyl)- 1-phenyl-3- azabicyclo[3.1.0]hexane-3- carboxamide | m/z = 370.2 [M + H]⁺ |
| | mixture of enantiomers | | |
| 95 | 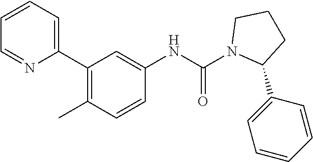 [SMILES: Cc1ccc(NC(=O)N2CCC[C@@H]2c2ccccc2)cc1-c1ccccn1] | (2R)-N-(4-methyl-3-pyridin-2- ylphenyl)-2-phenylpyrrolidine-1- carboxamide | m/z = 358.2 [M + H]⁺ |
| 96 | 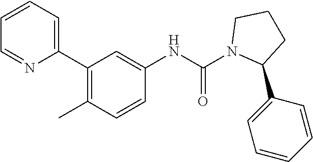 [SMILES: Cc1ccc(NC(=O)N2CCC[C@H]2c2ccccc2)cc1-c1ccccn1] | (2S)-N-(4-methyl-3-pyridin-2- ylphenyl)-2-phenylpyrrolidine-1- carboxamide | m/z = 358.2 [M + H]⁺ |
| 97 | 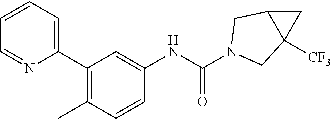 [SMILES: Cc1ccc(NC(=O)N2CC3CC3(C(F)(F)F)C2)cc1-c1ccccn1] | N-(4-methyl-3-pyridin-2-ylphenyl)- 1-(trifluoromethyl)-3- azabicyclo[3.1.0]hexane-3- carboxamide | m/z = 362.1 [M + H]⁺ |
| | mixture of enantiomers | | |
| 98 | 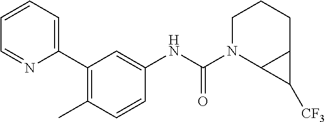 [SMILES: Cc1ccc(NC(=O)N2CCCC3C2C3C(F)(F)F)cc1-c1ccccn1] | N-(4-methyl-3-pyridin-2-ylphenyl)- 7-(trifluoromethyl)-2- azabicyclo[4.1.0]heptane-2- carboxamide | m/z = 376.1 [M + H]⁺ |
| | mixture of diastereomers | | |
| 99 | 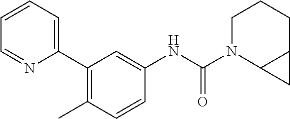 [SMILES: Cc1ccc(NC(=O)N2CCCC3CC32)cc1-c1ccccn1] | N-(4-methyl-3-pyridin-2-ylphenyl)- 2-azabicyclo[4.1.0]heptane-2- carboxamide | m/z = 308.1 [M + H]⁺ |
| | mixture of enantiomers | | |
| 100 | 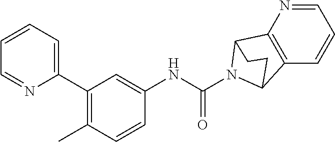 [SMILES: Cc1ccc(NC(=O)N2C3CCC2c2ncccc23)cc1-c1ccccn1] | N-(4-methyl-3-pyridin-2-ylphenyl)- 3,11- diazatricyclo[6.2.1.02,7]undeca- 2,4,6-triene-11-carboxamide | m/z = 357.2 [M + H]⁺ |
| | mixture of enantiomers | | |
| 101 | 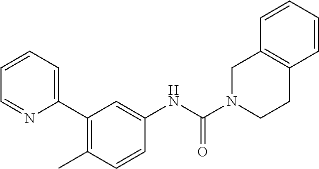 [SMILES: Cc1ccc(NC(=O)N2CCc3ccccc3C2)cc1-c1ccccn1] | N-(4-methyl-3-pyridin-2-ylphenyl)- 3,4-dihydro-1H-isoquinoline-2- carboxamide | m/z = 344.1 [M + H]⁺ |
| 102 | 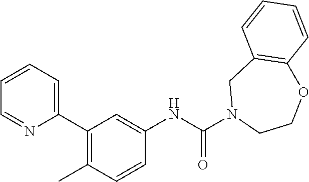 [SMILES: Cc1ccc(NC(=O)N2CCOc3ccccc3C2)cc1-c1ccccn1] | N-(4-methyl-3-pyridin-2-ylphenyl)- 3,5-dihydro-2H-1,4- benzoxazepine-4-carboxamide | m/z = 360.1 [M + H]⁺ |
| 103 | 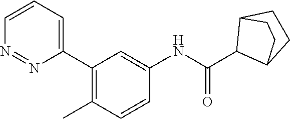 [SMILES: Cc1ccc(NC(=O)C2C3CCC2CC3)cc1-c1cccnn1] | N-(4-methyl-3-pyridazin-3- ylphenyl)bicyclo[2.2.1]heptane-7- carboxamide | m/z = 308.2 [M + H]⁺ |

### Examples 104 and 105

### (1R,4S,7R)—N-(4-methyl-3-(pyridin-2-yl)phenyl)-2-(2,2,2-trifluoroethyl)-2-azabicyclo[2.2.1]heptane-7-carboxamide and (1S,4R,7S)—N-(4-methyl-3-(pyridin-2-yl)phenyl)-2-(2,2,2-trifluoroethyl)-2-azabicyclo[2.2.1]heptane-7-carboxamide

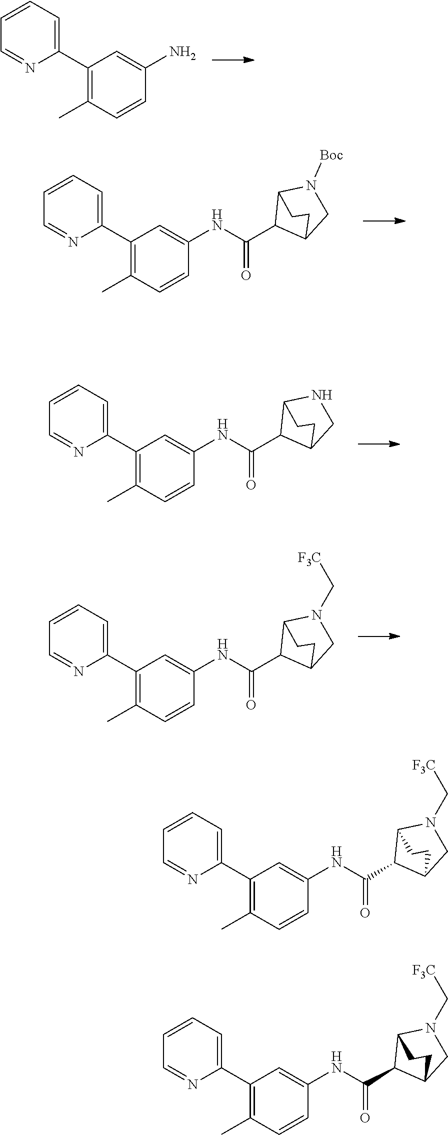 [SMILES: Cc1ccc(N)cc1-c1ccccn1 ; Cc1ccc(NC(=O)C2C3CCC2N(C(=O)OC(C)(C)C)C3)cc1-c1ccccn1 ; Cc1ccc(NC(=O)C2C3CCC2NC3)cc1-c1ccccn1 ; Cc1ccc(NC(=O)C2C3CCC2N(CC(F)(F)F)C3)cc1-c1ccccn1 ; Cc1ccc(NC(=O)[C@H]2[C@H]3CC[C@@H]2N(CC(F)(F)F)C3)cc1-c1ccccn1 ; Cc1ccc(NC(=O)[C@@H]2[C@@H]3CC[C@H]2N(CC(F)(F)F)C3)cc1-c1ccccn1]

tert-butyl 7-((4-methyl-3-(pyridin-2-yl)phenyl)carbamoyl)-2-azabicyclo[2.2.1]heptane-2-carboxylate: To a solution of 2-tert-butoxycarbonyl-2-azabicyclo[2.2.1]heptane-7-carboxylic acid (200 mg, 0.82 mmol) and 4-methyl-3-(2-pyridyl)aniline (183 mg, 0.99 mmol) in pyridine (4 mL) was added EDCI (317 mg, 1.66 mmol) at 0° C. under N₂. The mixture was stirred at 20° C. for 2 h. The reaction mixture was concentrated under reduced pressure and the resulting residue was purified by silica gel column chromatography (PE:EtOAc=10:1 to 1:1) to give the titled compound. LCMS: m/z=408.3 [M+H]⁺.

N-(4-methyl-3-(pyridin-2-yl)phenyl)-2-azabicyclo[2.2.1]heptane-7-carboxamide: A solution of tert-butyl 7-((4-methyl-3-(pyridin-2-yl)phenyl)carbamoyl)-2-azabicyclo[2.2.1]heptane-2-carboxylate (270 mg, 0.66 mmol) in HCl/EtOAc (4 mL) was stirred at 25° C. for 2 h. The reaction mixture was concentrated under reduced pressure to provide the titled compound. LCMS: m/z=308.2 [M+H]⁺.

trans-N-(4-methyl-3-(pyridin-2-yl)phenyl)-2-(2,2,2-trifluoroethyl)-2-azabicyclo[2.2.1]heptane-7-carboxamide: To a solution of N-[4-methyl-3-(2-pyridyl)phenyl]-2-azabicyclo[2.2.1]heptane-7-carboxamide (100 mg, 0.32 mmol) in THF (3 mL) was added DIEA (126 mg, 0.97 mmol) and 2,2,2-trifluoroethyl trifluoromethanesulfonate (113 mg, 0.48 mmol) at 0° C. under N₂. The mixture was stirred at 20° C. for 15 h. The reaction mixture was diluted with H₂O (5 mL) and extracted with EtOAc (3×5 mL). The combined organic layers were washed with brine (5 mL), dried over anhydrous Na₂SO₄, filtered and concentrated under reduced pressure to give the titled compound as a mixture of enantiomers. LCMS: m/z=390.0 [M+H]⁺.

The mixture of enantiomers was separated by SFC (Column: DAICEL CHIRALCEL OD (250 mm×30 mm, 10 m); Mobile phase: A: CO₂, B: 0.1% NH₃H₂O in MeOH; B % in A: 10%-35%, 12 min; Flow rate: 35 g/min; Wavelength: 220 nm; Column temperature: 35° C.; System back pressure: 120 bar) to provide trans-N-(4-methyl-3-(pyridin-2-yl)phenyl)-2-(2,2,2-trifluoroethyl)-2-azabicyclo[2.2.1]heptane-7-carboxamide (Example 105) as the first eluting peak isolated as a single unknown enantiomer. LCMS: m/z=389.9 [M+H]⁺. Further elution provided trans-N-(4-methyl-3-(pyridin-2-yl)phenyl)-2-(2,2,2-trifluoroethyl)-2-azabicyclo[2.2.1]heptane-7-carboxamide (Example 104) as the second eluting peak isolated as a single unknown enantiomer. LCMS: m/z=389.9 [M+H]⁺.

The following compound was, or can be, made via similar procedures as those described above.

| Ex. | Structure | Name | LCMS |
|---|---|---|---|
| 106 | 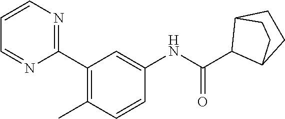 [SMILES: Cc1ccc(NC(=O)C2C3CCC2CC3)cc1-c1ncccn1] | N-(4-methyl-3-pyrimidin-2- ylphenyl)bicyclo[2.2.1]heptane-7- carboxamide | m/z = 308.2 [M + H]⁺ |

### Examples 107, 108, 109, and 110

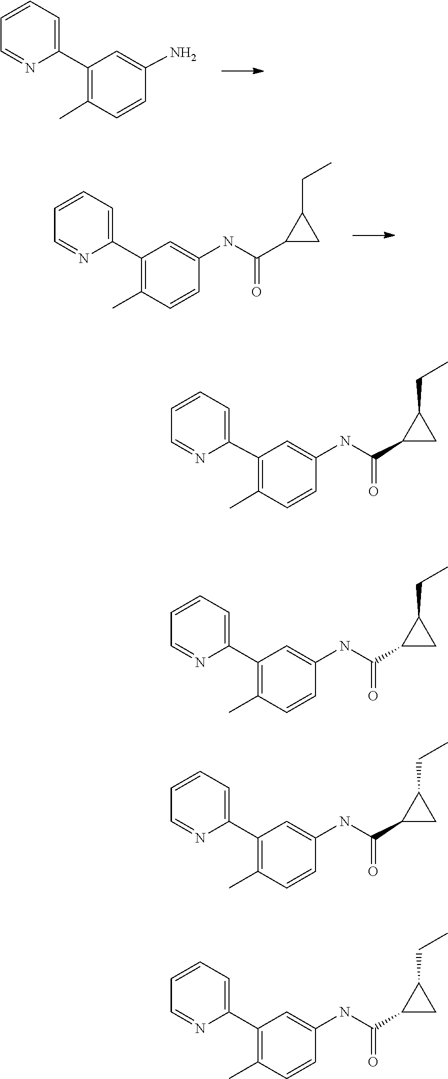

2-ethyl-N-(4-methyl-3-(pyridin-2-yl)phenyl)cyclopropanecarboxamide: To a solution of 4-methyl-3-(2-pyridyl)aniline (194 mg, 1.05 mmol) in pyridine (5 mL) was added 2-ethylcyclopropanecarboxylic acid (100 mg, 0.88 mmol) and EDCI (336 mg, 1.75 mmol) at 20° C. under N₂. The mixture was stirred at 20° C. for 2 h. The reaction mixture was diluted with H₂O (10 mL) and extracted with EtOAc (3×5 mL). The combined organic layers were washed with brine (10 mL), dried over anhydrous Na₂SO₄, filtered and concentrated under reduced pressure. The resulting residue was purified by prep-TLC (SiO₂, EtOAc) to provide the titled compound as a mixture of diastereomers. LCMS: m/z=281.1 [M+H]⁺.

(1R,2S)-2-ethyl-N-(4-methyl-3-(pyridin-2-yl)phenyl)cyclopropanecarboxamide and (1R,2R)-2-ethyl-N-(4-methyl-3-(pyridin-2-yl)phenyl)cyclopropanecarboxamide and (1S,2S)-2-ethyl-N-(4-methyl-3-(pyridin-2-yl)phenyl)cyclopropanecarboxamide and (1S,2R)-2-ethyl-N-(4-methyl-3-(pyridin-2-yl)phenyl)cyclopropanecarboxamide: The diastereomeric mixture was separated by SFC (Instrument: pre-SFC-9; Column: DAICEL CHIRALPAK AD (250 mm×30 mm, 10 m); Mobile phase: A: CO₂, B: 0.1% NH₃H₂O in IPA; B % in A: 15%-45%, 14 min; Flow rate: 70 mL/min; Wavelength: 220 nm; Column temperature: 35° C.; System back pressure: 100 bar) to provide cis-2-ethyl-N-(4-methyl-3-pyridin-2-ylphenyl)cyclopropane-1-carboxamide (Example 110) as the first eluting peak isolated as a single unknown enantiomer. LCMS: m/z=281.2 [M+H]⁺. Further elution provided trans-2-ethyl-N-(4-methyl-3-pyridin-2-ylphenyl)cyclopropane-1-carboxamide (Example 108) (Example 107) as the second eluting peak isolated as a mixture of enantiomers. Further elution provided cis-2-ethyl-N-(4-methyl-3-pyridin-2-ylphenyl)cyclopropane-1-carboxamide (Example 109) as the third eluting peak isolated as a single unknown enantiomer. LCMS: m/z=281.1 [M+H]⁺. Purification of the mixture of trans enantiomers by SFC (Column: DAICEL CHIRALCEL OD (250 mm×30 mm×10 n); Mobile phase: A: CO₂, B: 0.1% NH₃H₂O in IPA; B3% in A: 33%-33%, 15 min; Flow rate: 70 mL/min; Wavelength: 220 nm; Column temperature: 35° C.; System back pressure: 100 bar) provided trans-2-ethyl-N-(4-methyl-3-pyridin-2-ylphenyl)cyclopropane-1-carboxamide (Example 108) as the first eluting peak isolated as a single unknown enantiomer. LCMS: m/z=281.1 [M+H]. Further elution provided trans-2-ethyl-N-(4-methyl-3-pyridin-2-ylphenyl)cyclopropane-1-carboxamide (Example 107) as the second eluting peak isolated as a single unknown enantiomer. LCMS: m/z=281.1 [M+H]⁺.

The following compounds were, or can be, made via similar procedures as those described above.

| Ex. | Structure | Name | LCMS |
|---|---|---|---|
| 111 | 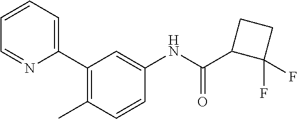 [SMILES: Cc1ccc(NC(=O)C2CCC2(F)F)cc1-c1ccccn1] | 2,2-difluoro-N-(4-methyl-3- pyridin-2-ylphenyl)cyclobutane- 1-carboxamide | m/z = 303.1 [M + H]⁺ |
| | mixture of enantiomers | | |
| 112 | 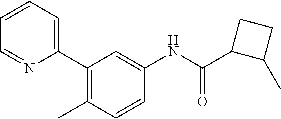 [SMILES: Cc1ccc(NC(=O)C2CCC2C)cc1-c1ccccn1] | 2-methyl-N-(4-methyl-3-pyridin- 2-ylphenyl)cyclobutane-1- carboxamide | m/z = 281.2 [M + H]⁺ |
| | mixture of diastereomers | | |
| 113 | 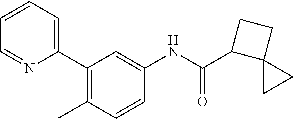 [SMILES: Cc1ccc(NC(=O)C2CCC23CC3)cc1-c1ccccn1] | N-(4-methyl-3-pyridin-2- ylphenyl)spiro[2.3]hexane-6- carboxamide | m/z = 293.2 [M + H]⁺ |
| | mixture of enantiomers | | |
| 114 | 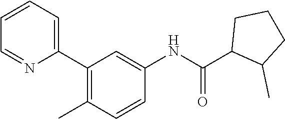 [SMILES: Cc1ccc(NC(=O)C2CCCC2C)cc1-c1ccccn1] | 2-methyl-N-(4-methyl-3-pyridin- 2-ylphenyl)cyclopentane-1- carboxamide | m/z = 295.2 [M + H]⁺ |
| | mixture of enantiomers | | |
| 115 | 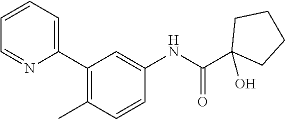 [SMILES: Cc1ccc(NC(=O)C2(O)CCCC2)cc1-c1ccccn1] | 1-hydroxy-N-(4-methyl-3- pyridin-2-ylphenyl)cyclopentane- 1-carboxamide | m/z = 297.1 [M + H]⁺ |

### Examples 116 and 117

### trans-N-(4-methyl-3-pyridin-2-ylphenyl)bicyclo[2.1.0]pentane-2-carboxamide and cis-N-(4-methyl-3-pyridin-2-ylphenyl)bicyclo[2.1.0]pentane-2-carboxamide

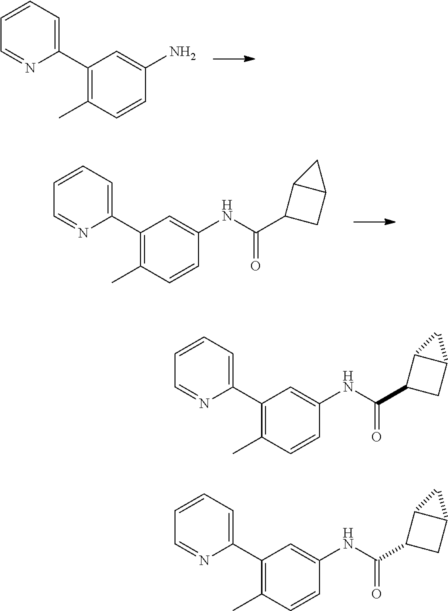

N-(4-methyl-3-(pyridin-2-yl)phenyl)bicyclo[2.1.0]pentane-2-carboxamide: To a solution of 4-methyl-3-(pyridin-2-yl)aniline (50 mg, 0.27 mmol) in pyridine (2 mL) was added bicyclo[2.1.0]pentane-2-carboxylic acid (37 mg, 0.33 mmol) and EDCI (104 mg, 0.54 mmol) at 0° C. under N₂. The mixture was stirred at 20° C. for 12 h. The reaction mixture was concentrated under reduced pressure and the resulting residue was purified prep-HPLC (Waters Xbridge Prep OBD (C18 150 mm×40 mm×10 m); Mobile phase: A: 10 mM NH₄HCO₃ in water; B: MeCN; B % in A: 30%-50%, 8 min) to provide cis-N-(4-methyl-3-pyridin-2-ylphenyl)bicyclo[2.1.0]pentane-2-carboxamide (Example 117) as the first eluting peak isolated as a mixture of enantiomers. LCMS: m/z=279.2 [M+H]⁺. Further elution provided trans-N-(4-methyl-3-pyridin-2-ylphenyl)bicyclo[2.1.0]pentane-2-carboxamide (Example 116) as the second eluting peak isolated as a mixture of enantiomers. LCMS: m/z=279.2 [M+H]⁺.

The following compounds were, or can be, made via similar procedures as those described above.

| Ex. | Structure | Name | LCMS |
|---|---|---|---|
| 118 | 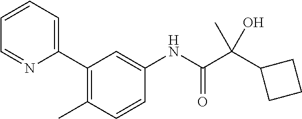 [SMILES: Cc1ccc(NC(=O)C(C)(O)C2CCC2)cc1-c1ccccn1] | 2-cyclobutyl-2-hydroxy-N-(4-methyl- 3-pyridin-2-ylphenyl)propanamide | m/z = 311.2 [M + H]⁺ |
| | mixture of enantiomers | | |
| 119 | 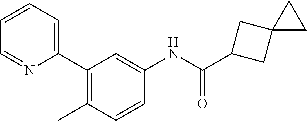 [SMILES: Cc1ccc(NC(=O)C2CC3(CC3)C2)cc1-c1ccccn1] | N-(4-methyl-3-pyridin-2- ylphenyl)spiro[2.3]hexane-5- carboxamide | m/z = 293.1 [M + H]⁺ |
| 120 | 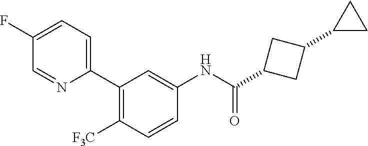 [SMILES: O=C(Nc1ccc(C(F)(F)F)c(-c2ccc(F)cn2)c1)[C@H]1C[C@@H](C2CC2)C1] | cis-3-cyclopropyl-N-[3-(5- fluoropyridin-2-yl)-4- (trifluoromethyl)phenyl]cyclobutane- 1-carboxamide | m/z = 379.1 [M + H]⁺ |

### Examples 121 and 122

### trans-3-cyclopropyl-N-(4-methyl-3-pyridin-2-ylphenyl)cyclobutane-1-carboxamide (Example 121) and cis-3-cyclopropyl-N-(4-methyl-3-pyridin-2-ylphenyl)cyclobutane-1-carboxamide (Example

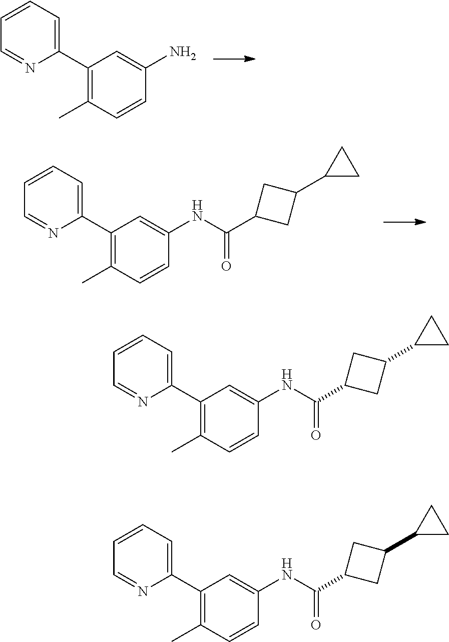

3-cyclopropyl-N-(4-methyl-3-(pyridin-2-yl)phenyl)cyclobutanecarboxamide: To a mixture of 4-methyl-3-(2-pyridyl)aniline (100 mg, 0.54 mmol) and 3-cyclopropylcyclobutanecarboxylic acid (76 mg, 0.54 mmol) in pyridine (5 mL) was added EDCI (208 mg, 1.09 mmol) at 0° C. under N₂. The mixture was stirred at 25° C. for 30 min. The reaction mixture was diluted with H₂O (5 mL) and extracted with DCM (3×5 mL). The combined organic layers were dried over anhydrous Na₂SO₄, filtered and concentrated under reduced pressure. The resulting residue was purified by prep-HPLC (Waters Xbridge BEH C18 100×30 mm×10 m; mobile phase: A: 10 mM NH₄HCO₃ in water, B: MeCN; B in A: 40%-70%, 10 min) to provide the titled compound as a mixture of diastereomers. LCMS: m/z=307.2 [M+H]⁺.

The mixture of diastereomers was separated by SFC (Column: DAICEL CHIRALPAK AD (250 mm×30 mm×10 m); Mobile phase: A: CO₂, B: 0.1% NH₃H₂O in EtOH; B % in A: 20%-20%, 10 min; Flow rate: 70 g/min; Wavelength: 220 nm; Column temperature: 40° C.; System back pressure: 100 bar) to provide cis-3-cyclopropyl-N-(4-methyl-3-pyridin-2-ylphenyl)cyclobutane-1-carboxamide (Example 122) as the first eluting peak. LCMS m/z=307.2 [M+H]⁺. Further elution provided trans-3-cyclopropyl-N-(4-methyl-3-pyridin-2-ylphenyl)cyclobutane-1-carboxamide (Example 121) as the second eluting peak. LCMS m/z=307.2 [M+H]⁺.

The following compounds were, or can be, made via similar procedures as those described above. Example 124 was prepared using commercially available carboxylic acid (CAS: 1258652-18-4). Example 128 was prepared using commercially available carboxylic acid (CAS: 78376-99-5).

| Ex. | Structure | Name | LCMS |
|---|---|---|---|
| 123 | 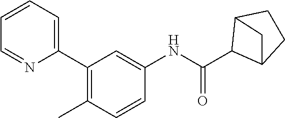 [SMILES: Cc1ccc(NC(=O)C2C3CCC2C3)cc1-c1ccccn1] | N-(4-methyl-3-pyridin-2- ylphenyl)bicyclo[2.1.1]hexane-5- carboxamide | m/z = 293.1 [M + H]⁺ |
| 124 | 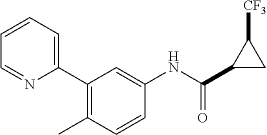 [SMILES: Cc1ccc(NC(=O)[C@@H]2C[C@@H]2C(F)(F)F)cc1-c1ccccn1] | cis-N-(4-methyl-3-pyridin-2- ylphenyl)-2- (trifluoromethyl)cyclopropane-1- carboxamide | m/z = 321.1 [M + H]⁺ |
| | mixture of enantiomers | | |
| 125 | 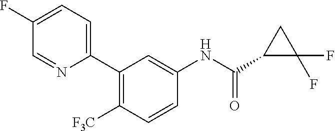 [SMILES: O=C(Nc1ccc(C(F)(F)F)c(-c2ccc(F)cn2)c1)[C@@H]1CC1(F)F] | (1S)-2,2-difluoro-N-[3-(5- fluoropyridin-2-yl)-4- (trifluoromethyl)phenyl]cyclopro- pane-1-carboxamide | m/z = 361.1 [M + H]⁺ |
| 126 | 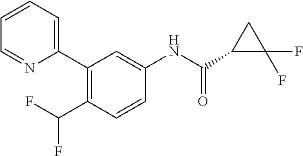 [SMILES: O=C(Nc1ccc(C(F)F)c(-c2ccccn2)c1)[C@@H]1CC1(F)F] | (1S)-N-[4-(difluoromethyl)-3- pyridin-2-ylphenyl]-2,2- difluorocyclopropane-1- carboxamide | m/z = 325.1 [M + H]⁺ |
| 127 | 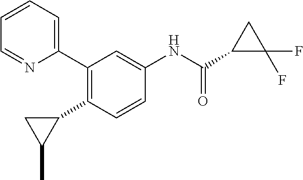 [SMILES: C[C@H]1C[C@@H]1c1ccc(NC(=O)[C@@H]2CC2(F)F)cc1-c1ccccn1] | (1S)-2,2-difluoro-N-[4-[trans-2- methylcyclopropyl]-3-pyridin-2- ylphenyl]cyclopropane-1- carboxamide | m/z = 329.1 [M + H]⁺ |
| | mixture of diastereomers | | |
| 128 | 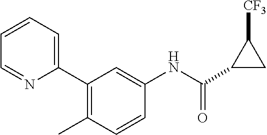 [SMILES: Cc1ccc(NC(=O)[C@H]2C[C@@H]2C(F)(F)F)cc1-c1ccccn1] | trans-N-(4-methyl-3-pyridin-2- ylphenyl)-2- (trifluoromethyl)cyclopropane-1- carboxamide | m/z = 321.1 [M + H]⁺ |
| | mixture of enantiomers | | |
| 129 | 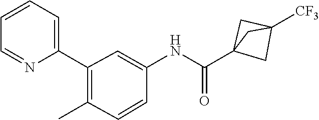 [SMILES: Cc1ccc(NC(=O)C23CC(C(F)(F)F)(C2)C3)cc1-c1ccccn1] | N-(4-methyl-3-pyridin-2-ylphenyl)- 3-(trifluoromethyl)bicyclo[1.1.1] pentane-1-carboxamide | m/z = 347.1 [M + H]⁺ |
| 130 | 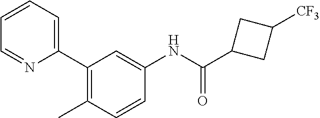 [SMILES: Cc1ccc(NC(=O)C2CC(C(F)(F)F)C2)cc1-c1ccccn1] | N-(4-methyl-3-pyridin-2-ylphenyl)- 3-(trifluoromethyl)cyclobutane-1- carboxamide | m/z = 335.1 [M + H]⁺ |
| | mixture of diastereomers | | |
| 131 | 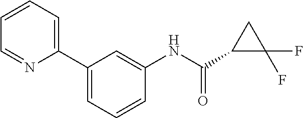 [SMILES: O=C(Nc1cccc(-c2ccccn2)c1)[C@@H]1CC1(F)F] | (1S)-2,2-difluoro-N-(3-pyridin-2- ylphenyl)cyclopropane-1- carboxamide | m/z = 275.1 [M + H]⁺ |
| 132 | 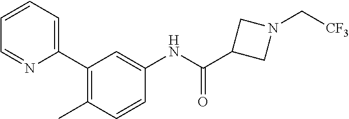 [SMILES: Cc1ccc(NC(=O)C2CN(CC(F)(F)F)C2)cc1-c1ccccn1] | N-(4-methyl-3-pyridin-2-ylphenyl)- 1-(2,2,2-trifluoroethyl)azetidine-3- carboxamide | m/z = 350.1 [M + H]⁺ |
| 133 | 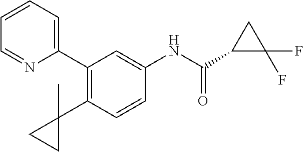 [SMILES: CC1(c2ccc(NC(=O)[C@@H]3CC3(F)F)cc2-c2ccccn2)CC1] | (1S)-2,2-difluoro-N-[4-(1- methylcyclopropyl)-3-pyridin-2- ylphenyl]cyclopropane-1- carboxamide | m/z = 329.1 [M + H]⁺ |
| 134 | 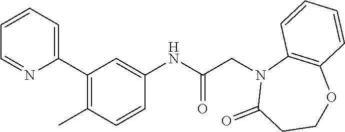 [SMILES: Cc1ccc(NC(=O)CN2C(=O)CCOc3ccccc32)cc1-c1ccccn1] | N-(4-methyl-3-pyridin-2-ylphenyl)- 2-(4-oxo-2,3-dihydro-1,5- benzoxazepin-5-yl)acetamide | m/z = 388.1 [M + H]⁺ |
| 135 | 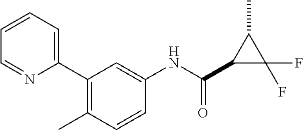 | trans-2,2-difluoro-3-methyl-N-(4- methyl-3-pyridin-2- ylphenyl)cyclopropane-1- carboxamide | m/z = 303.1 [M + H]⁺ |
| | mixture of enantiomers | | |
| 136 | 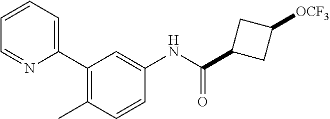 [SMILES: Cc1ccc(NC(=O)[C@H]2C[C@@H](OC(F)(F)F)C2)cc1-c1ccccn1] | cis-N-(4-methyl-3-pyridin-2- ylphenyl)-3- (trifluoromethoxy)cyclobutane-1- carboxamide | m/z = 351.1 [M + H]⁺ |

### Examples 137 and 138 and 139 and 140

### (1R,2S,4S)—N-(4-methyl-3-(pyridin-2-yl)phenyl)bicyclo[2.2.1]heptane-2-carboxamide and (1S,2S,4R)—N-(4-methyl-3-(pyridin-2-yl)phenyl)bicyclo[2.2.1]heptane-2-carboxamide and (1R,2R,4S)—N-(4-methyl-3-(pyridin-2-yl)phenyl)bicyclo[2.2.1]heptane-2-carboxamide and (1S,2R,4R)—N-(4-methyl-3-(pyridin-2-yl)phenyl)bicyclo[2.2.1]heptane-2-carboxamide

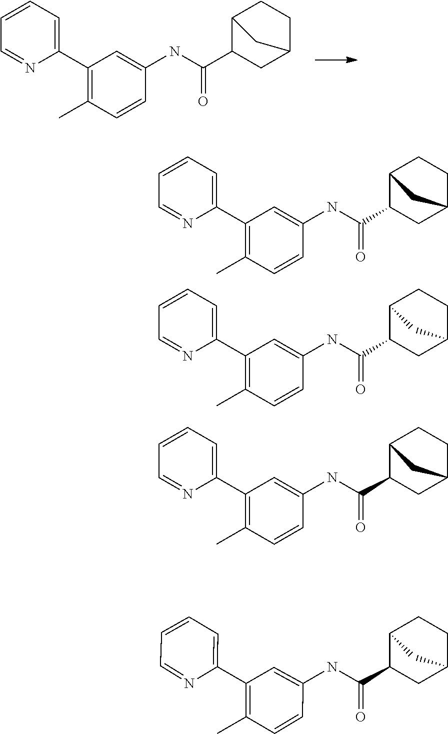

The diastereomeric mixture from Example 147 was separated by SFC (Column: DAICEL CHIRALCEL OD (250 mm×30 mm×10 μm); Mobile phase: A: CO₂, B: 0.1% NH₃H₂O in IPA; B % in A: 40%-40%, 9 min; Flow rate: 67 mL/min; Wavelength: 220 nm; Column temperature: 35° C.; System back pressure: 100 bar) to provide trans-(—N-(4-methyl-3-pyridin-2-ylphenyl)bicyclo[2.2.1]heptane-2-carboxamide (Example 140) as the first eluting peak isolated as a single unknown enantiomer. LCMS: m/z=307.2 [M+H]⁺. Further elution provided cis-N-(4-methyl-3-pyridin-2-ylphenyl)bicyclo[2.2.1]heptane-2-carboxamide (Example 138) as the second eluting peak isolated as a single unknown enantiomer. LCMS: m/z=307.2 [M+H]⁺. Further elution provided cis-N-(4-methyl-3-pyridin-2-ylphenyl)bicyclo[2.2.1]heptane-2-carboxamide (Example 139) as the third eluting peak isolated as a single unknown enantiomer. LCMS: m/z=307.2 [M+H]⁺. Further elution provided trans-N-(4-methyl-3-pyridin-2-ylphenyl)bicyclo[2.2.1]heptane-2-carboxamide (Example 137) as the fourth eluting peak isolated as a single unknown enantiomer.

The following compounds were, or can be, made via similar procedures as those described above.

| Ex. | Structure | Name | LCMS |
|---|---|---|---|
| 141 | 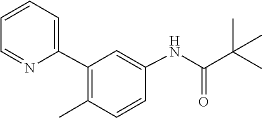 [SMILES: Cc1ccc(NC(=O)C(C)(C)C)cc1-c1ccccn1] | 2,2-dimethyl-N-(4-methyl-3-pyridin- 2-ylphenyl)propanamide | m/z = 269.2 [M + H]⁺ |
| 142 | 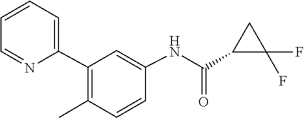 [SMILES: Cc1ccc(NC(=O)[C@@H]2CC2(F)F)cc1-c1ccccn1] | (1S)-2,2-difluoro-N-(4-methyl-3- pyridin-2-ylphenyl)cyclopropane-1- carboxamide | m/z = 289.1 [M + H]⁺ |
| 143 | 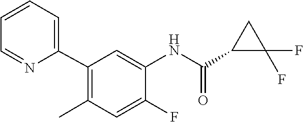 [SMILES: Cc1cc(F)c(NC(=O)[C@@H]2CC2(F)F)cc1-c1ccccn1] | 2,2-difluoro-N-(2-fluoro-4-methyl-5- pyridin-2-ylphenyl)cyclopropane-1- carboxamide | m/z = 307.1 [M + H]⁺ |
| 144 | 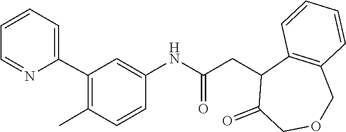 [SMILES: Cc1ccc(NC(=O)CC2C(=O)COCc3ccccc32)cc1-c1ccccn1] | N-(4-methyl-3-pyridin-2-ylphenyl)-2- (2-oxo-5H-4,1-benzoxazepin-1- yl)acetamide | m/z = 388.1 [M + H]⁺ |
| 145 | 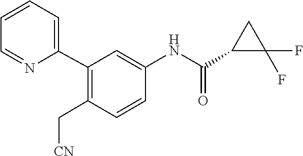 [SMILES: N#CCc1ccc(NC(=O)[C@@H]2CC2(F)F)cc1-c1ccccn1] | (1S)-N-[4-(cyanomethyl)-3-pyridin- 2-ylphenyl]-2,2- difluorocyclopropane-1-carboxamide | m/z = 314.1 [M + H]⁺ |
| 146 | 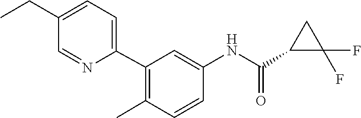 [SMILES: CCc1ccc(-c2cc(NC(=O)[C@@H]3CC3(F)F)ccc2C)nc1] | (1S)-N-[3-(5-ethylpyridin-2-yl)-4- methylphenyl]-2,2- difluorocyclopropane-1-carboxamide | m/z = 317.1 [M + H]⁺ |
| 147 | 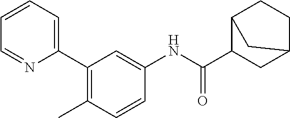 [SMILES: Cc1ccc(NC(=O)C2CC3CCC2C3)cc1-c1ccccn1] | N-(4-methyl-3-pyridin-2- ylphenyl)bicyclo[2.2.1]heptane-2- carboxamide | m/z = 307.1 [M + H]⁺ |
| | mixture of diastereomers | | |
| 148 | 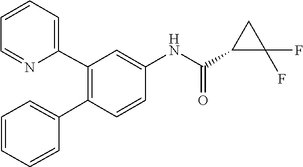 [SMILES: O=C(Nc1ccc(-c2ccccc2)c(-c2ccccn2)c1)[C@@H]1CC1(F)F] | (1S)-2,2-difluoro-N-(4-phenyl-3- pyridin-2-ylphenyl)cyclopropane-1- carboxamide | m/z = 351.1 [M + H]⁺ |
| 149 | 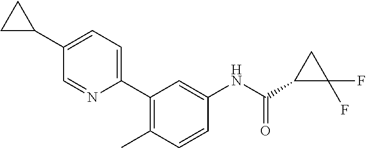 [SMILES: Cc1ccc(NC(=O)[C@@H]2CC2(F)F)cc1-c1ccc(C2CC2)cn1] | (1S)-N-[3-(5-cyclopropylpyridin-2- yl)-4-methylphenyl]-2,2- difluorocyclopropane-1-carboxamide | m/z = 329.1 [M + H]⁺ |
| 150 | 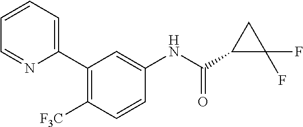 [SMILES: O=C(Nc1ccc(C(F)(F)F)c(-c2ccccn2)c1)[C@@H]1CC1(F)F] | (1S)-2,2-difluoro-N-[3-pyridin-2-yl- 4-(trifluoromethyl)phenyl] cyclopropane-1-carboxamide | m/z = 343.0 [M + H]⁺ |
| 151 | 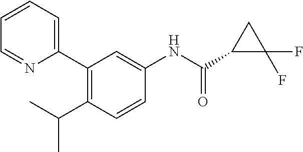 [SMILES: CC(C)c1ccc(NC(=O)[C@@H]2CC2(F)F)cc1-c1ccccn1] | (1S)-2,2-difluoro-N-(4-propan-2-yl- 3-pyridin-2-ylphenyl)cyclopropane- 1-carboxamide | m/z = 317.1 [M + H]⁺ |
| 152 | 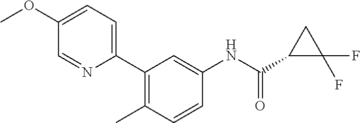 [SMILES: COc1ccc(-c2cc(NC(=O)[C@@H]3CC3(F)F)ccc2C)nc1] | (1S)-2,2-difluoro-N-[3-(5- methoxypyridin-2-yl)-4- methylphenyl]cyclopropane-1- carboxamide | m/z = 319.1 [M + H]⁺ |
| 153 | 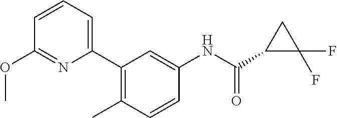 [SMILES: COc1cccc(-c2cc(NC(=O)[C@@H]3CC3(F)F)ccc2C)n1] | (1S)-2,2-difluoro-N-[3-(6- methoxypyridin-2-yl)-4- methylphenyl]cyclopropane-1- carboxamide | m/z = 319.1 [M + H]⁺ |
| 154 | 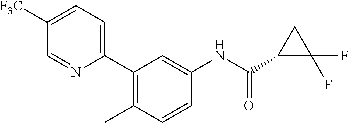 [SMILES: Cc1ccc(NC(=O)[C@@H]2CC2(F)F)cc1-c1ccc(C(F)(F)F)cn1] | (1S)-2,2-difluoro-N-[4-methyl-3-[5- (trifluoromethyl)pyridin-2- yl]phenyl]cyclopropane-1- carboxamide | m/z = 357.1 [M + H]⁺ |
| 155 | 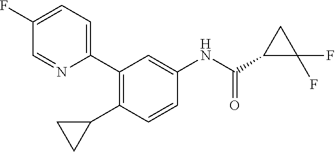 [SMILES: O=C(Nc1ccc(C2CC2)c(-c2ccc(F)cn2)c1)[C@@H]1CC1(F)F] | (1S)-N-[4-cyclopropyl-3-(5- fluoropyridin-2-yl)phenyl]-2,2- difluorocyclopropane-1-carboxamide | m/z = 333.1 [M + H]⁺ |
| 156 | 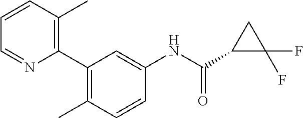 [SMILES: Cc1ccc(NC(=O)[C@@H]2CC2(F)F)cc1-c1ncccc1C] | (1S)-2,2-difluoro-N-[4-methyl-3-(3- methylpyridin-2- yl)phenyl]cyclopropane-1- carboxamide | m/z = 303.1 [M + H]⁺ |
| 157 | 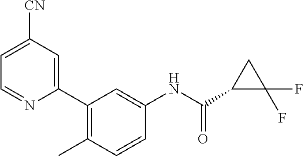 [SMILES: Cc1ccc(NC(=O)[C@@H]2CC2(F)F)cc1-c1cc(C#N)ccn1] | (1S)-N-[3-(4-cyanopyridin-2-yl)-4- methylphenyl]-2,2- difluorocyclopropane-1-carboxamide | m/z = 314.1 [M + H]⁺ |
| 158 | 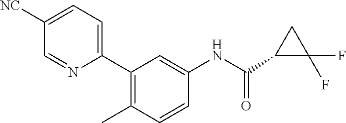 [SMILES: Cc1ccc(NC(=O)[C@@H]2CC2(F)F)cc1-c1ccc(C#N)cn1] | (1S)-N-[3-(5-cyanopyridin-2-yl)-4- methylphenyl]-2,2- difluorocyclopropane-1-carboxamide | m/z = 314.1 [M + H]⁺ |
| 159 | 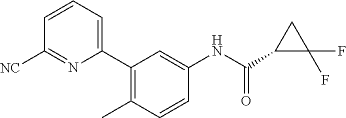 [SMILES: Cc1ccc(NC(=O)[C@@H]2CC2(F)F)cc1-c1cccc(C#N)n1] | (1S)-N-[3-(6-cyanopyridin-2-yl)-4- methylphenyl]-2,2- difluorocyclopropane-1-carboxamide | m/z = 314.1 [M + H]⁺ |
| 160 | 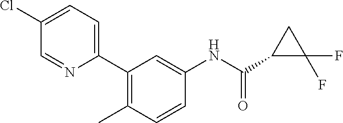 [SMILES: Cc1ccc(NC(=O)[C@@H]2CC2(F)F)cc1-c1ccc(Cl)cn1] | (1S)-N-[3-(5-chloropyridin-2-yl)-4- methylphenyl]-2,2- difluorocyclopropane-1-carboxamide | m/z = 323.0 [M + H]⁺ |
| 161 | 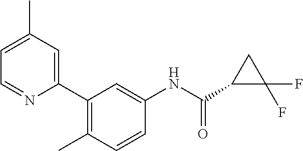 [SMILES: Cc1ccnc(-c2cc(NC(=O)[C@@H]3CC3(F)F)ccc2C)c1] | (1S)-2,2-difluoro-N-[4-methyl-3-(4- methylpyridin-2- yl)phenyl]cyclopropane-1- carboxamide | m/z = 303.1 [M + H]⁺ |
| 162 | 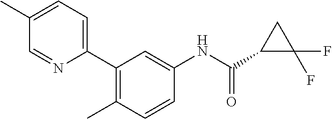 [SMILES: Cc1ccc(-c2cc(NC(=O)[C@@H]3CC3(F)F)ccc2C)nc1] | (1S)-2,2-difluoro-N-[4-methyl-3-(5- methylpyridin-2- yl)phenyl]cyclopropane-1- carboxamide | m/z = 303.1 [M + H]⁺ |
| 163 | 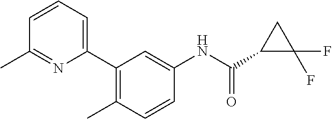 [SMILES: Cc1cccc(-c2cc(NC(=O)[C@@H]3CC3(F)F)ccc2C)n1] | (1S)-2,2-difluoro-N-[4-methyl-3-(6- methylpyridin-2- yl)phenyl]cyclopropane-1- carboxamide | m/z = 303.1 [M + H]⁺ |
| 164 | 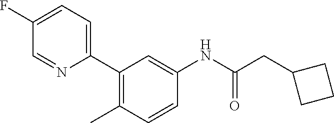 [SMILES: Cc1ccc(NC(=O)CC2CCC2)cc1-c1ccc(F)cn1] | 2-cyclobutyl-N-[3-(5-fluoropyridin- 2-yl)-4-methylphenyl]acetamide | m/z = 299.1 [M + H]⁺ |
| 165 | 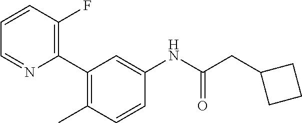 [SMILES: Cc1ccc(NC(=O)CC2CCC2)cc1-c1ncccc1F] | 2-cyclobutyl-N-[3-(3-fluoropyridin- 2-yl)-4-methylphenyl]acetamide | m/z = 299.1 [M + H]⁺ |
| 166 | 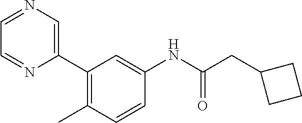 [SMILES: Cc1ccc(NC(=O)CC2CCC2)cc1-c1cnccn1] | 2-cyclobutyl-N-(4-methyl-3-pyrazin- 2-ylphenyl)acetamide | m/z = 282.1 [M + H]⁺ |
| 167 | 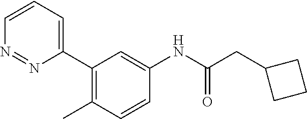 [SMILES: Cc1ccc(NC(=O)CC2CCC2)cc1-c1cccnn1] | 2-cyclobutyl-N-(4-methyl-3- pyridazin-3-ylphenyl)acetamide | m/z = 282.1 [M + H]⁺ |
| 168 | 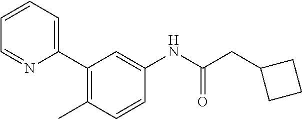 [SMILES: Cc1ccc(NC(=O)CC2CCC2)cc1-c1ccccn1] | 2-cyclobutyl-N-(4-methyl-3-pyridin- 2-ylphenyl)acetamide | m/z = 281.1 [M + H]⁺ |
| 169 | 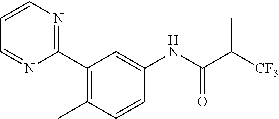 [SMILES: Cc1ccc(NC(=O)C(C)C(F)(F)F)cc1-c1ncccn1] | 3,3,3-trifluoro-2-methyl-N-(4- methyl-3-pyrimidin-2- ylphenyl)propanamide | m/z = 310.1 [M + H]⁺ |
| | mixture of enantiomers | | |

### Example 170

### (1R,3R,5R)—N-(3-(5-fluoropyrimidin-2-yl)-4-methylphenyl)-2-azabicyclo[3.1.0]hexane-3-carboxamide

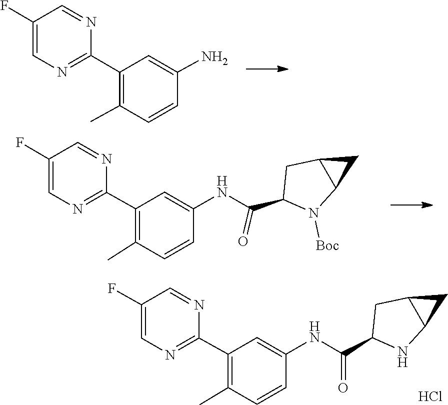

tert-butyl (1R,3R,5R)-3-((3-(5-fluoropyrimidin-2-yl)-4-methylphenyl)carbamoyl)-2-azabicyclo[3.1.0]hexane-2-carboxylate: To a mixture of 3-(5-fluoropyrimidin-2-yl)-4-methyl-aniline (670 mg, 3.30 mmol) and (1R,3R,5R)-2-tert-butoxycarbonyl-2-azabicyclo[3.1.0]hexane-3-carboxylic acid (500 mg, 2.20 mmol) in DMF (5 mL) at 0° C. under N₂ was added HATU (1.00 g, 2.64 mmol) and DIEA (1.14 g, 8.80 mmol). The mixture was warmed to 20° C. and stirred for 2 h. The reaction mixture was diluted with H₂O (10 mL) and extracted with EtOAc (3×10 mL). The combined organic layers were washed with brine (2×10 mL), dried over anhydrous Na₂SO₄, filtered and concentrated under reduced pressure. The resulting residue was purified by silica gel column chromatography (PE:EtOAc=3:1 to 5:1) to give the titled product. LCMS: m/z=313.2 [M-Boc+H]⁺.

(1R,3R,5R)—N-(3-(5-fluoropyrimidin-2-yl)-4-methylphenyl)-2-azabicyclo[3.1.0]hexane-3-carboxamide: A solution of tert-butyl (1R,3R,5R)-3-[[3-(5-fluoropyrimidin-2-yl)-4-methyl-phenyl]carbamoyl]-2-azabicyclo[3.1.0]hexane-2-carboxylate (300 mg, 0.73 mmol) in HCl/EtOAc (20 mL, 4 M) was stirred at 20° C. for 2 h and then the reaction mixture was concentrated under reduced pressure to give the titled compound. LCMS: m/z=313.1 [M+H]⁺.

### Example 171

### (1R,3R,5R)—N-(3-(5-fluoropyrimidin-2-yl)-4-methylphenyl)-2-((5-methyl-1,3,4-oxadiazol-2-yl)methyl)-2-azabicyclo[3.1.0]hexane-3-carboxamide

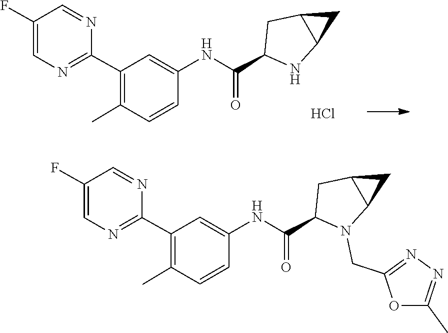 [SMILES: Cc1ccc(NC(=O)[C@H]2C[C@H]3C[C@H]3N2)cc1-c1ncc(F)cn1 ; Cl ; Cc1nnc(CN2[C@@H](C(=O)Nc3ccc(C)c(-c4ncc(F)cn4)c3)C[C@H]3C[C@H]32)o1]

To a solution of (1R,3R,5R)—N-[3-(5-fluoropyrimidin-2-yl)-4-methyl-phenyl]-2-azabicyclo[3.1.0]hexane-3-carboxamide hydrochloride (150 mg, 0.43 mmol) in DCM (5 mL) at 25° C. under N₂ was added 2-(chloromethyl)-5-methyl-1,3,4-oxadiazole (85 mg, 0.645 mmol), DIEA (222 mg, 1.72 mmol) and NaI (32 mg, 0.21 mmol). The reaction mixture was stirred at 25° C. for 12 h and then the reaction mixture was concentrated under reduced pressure. The resulting residue was purified by prep-HPLC (Waters Xbridge Prep OBD C18 150×40 mm×10 μm; mobile phase: A: 10 mM NH₄HCO₃ in water, B: MeCN; B % in A: 30%-60% over 8 min) to give the titled compound. LCMS: m/z=409.2 [M+H]⁺.

### Example 172

### (1R,3R,5R)—N-(3-(5-fluoropyrimidin-2-yl)-4-methylphenyl)-2-(3-(methylthio)-1,2,4-triazin-6-yl)-2-azabicyclo[3.1.0]hexane-3-carboxamide

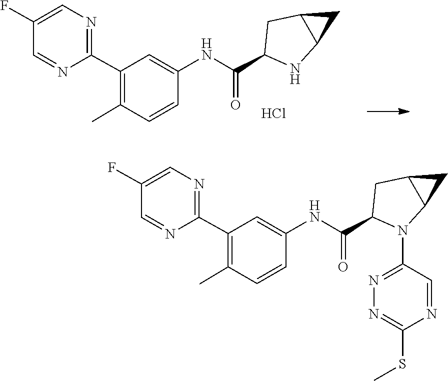 [SMILES: Cc1ccc(NC(=O)[C@H]2C[C@H]3C[C@H]3N2)cc1-c1ncc(F)cn1 ; Cl ; CSc1ncc(N2[C@@H](C(=O)Nc3ccc(C)c(-c4ncc(F)cn4)c3)C[C@H]3C[C@H]32)nn1]

(1R,3R,5R)—N-(3-(5-fluoropyrimidin-2-yl)-4-methylphenyl)-2-(3-(methylthio)-1,2,4-triazin-6-yl)-2-azabicyclo[3.1.0]hexane-3-carboxamide: To a solution of (1R,3R,5R)—N-(3-(5-fluoropyrimidin-2-yl)-4-methylphenyl)-2-azabicyclo[3.1.0]hexane-3-carboxamide hydrochloride (300 mg, 0.86 mmol) in EtOH (7 mL) at 20° C. was added DIEA (333 mg) and 6-chloro-3-(methylthio)-1,2,4-triazine (167 mg, 1.03 mmol). The mixture was heated to 80° C. and stirred for 6 h. The reaction mixture was filtered and concentrated under reduced pressure. The resulting residue was purified by silica gel column chromatography (EtOAc) to give the crude product which was further purified by prep-HPLC (Waters Xbridge BEH C18 100×30 mm×10 m; mobile phase: A: 10 mM NH₄HCO₃ in water, B: MeCN; B % in A: 35%-65%, 8 min) to give the titled compound. LCMS: m/z=438.1 [M+H]⁺.

### Example 173

### (1R,3R,5R)—N-(3-(5-fluoropyrimidin-2-yl)-4-methylphenyl)-2-(1,2,4-triazin-6-yl)-2-azabicyclo[3.1.0]hexane-3-carboxamide

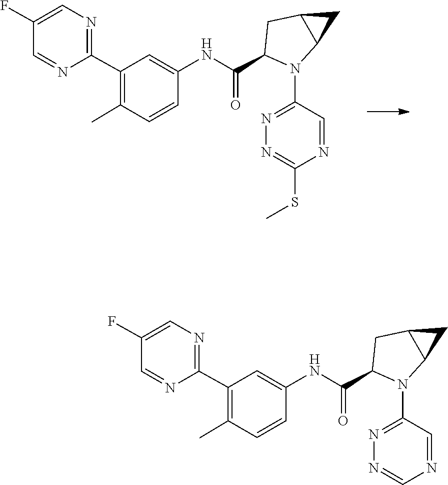 [SMILES: CSc1ncc(N2[C@@H](C(=O)Nc3ccc(C)c(-c4ncc(F)cn4)c3)C[C@H]3C[C@H]32)nn1 ; Cc1ccc(NC(=O)[C@H]2C[C@H]3C[C@H]3N2c2cncnn2)cc1-c1ncc(F)cn1]

(1R,3R,5R)—N-(3-(5-fluoropyrimidin-2-yl)-4-methylphenyl)-2-(1,2,4-triazin-6-yl)-2-azabicyclo[3.1.0]hexane-3-carboxamide: To a solution of (1R,3R,5R)—N-(3-(5-fluoropyrimidin-2-yl)-4-methylphenyl)-2-(3-(methylthio)-1,2,4-triazin-6-yl)-2-azabicyclo[3.1.0]hexane-3-carboxamide (80 mg, 0.18 mmol) in THF (2 mL) at 20° C. was added Et₃SiH (43 mg, 0.37 mmol) and PdCl₂ (3 mg, 0.02 mmol). The reaction mixture was stirred at 20° C. for 3 h and then the reaction was filtered and concentrated under reduced pressure. The resulting residue was purified by prep-HPLC (Waters Xbridge BEH C18 100×30 mm×10 m; mobile phase: A: 10 mM NH₄HCO₃ in water, B: MeCN; B % in A: 15%-45%, 8 min) to give the titled compound. LCMS: m/z=392.2 [M+H]⁺.

### Example 174

### (2S,4S)—N-(4-methyl-3-(pyridazin-3-yl)phenyl)-1-(pyrimidin-2-yl)-4-(trifluoromethyl)pyrrolidine-2-carboxamide

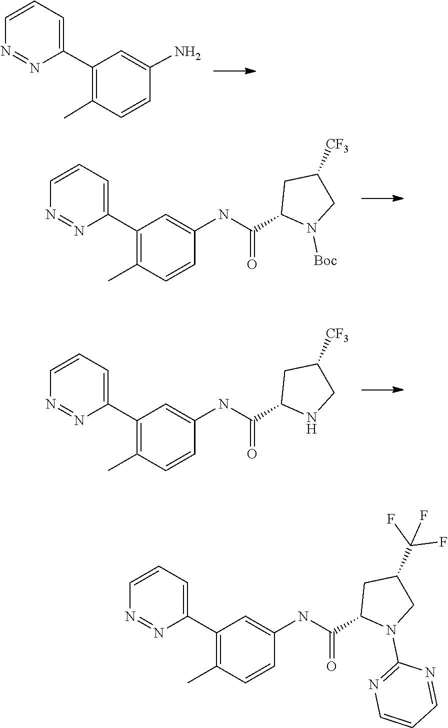

tert-butyl (2S,4S)-2-((4-methyl-3-(pyridazin-3-yl)phenyl)carbamoyl)-4-(trifluoromethyl)pyrrolidine-1-carboxylate: To a solution of 4-methyl-3-pyridazin-3-yl-aniline (181 mg, 0.97 mmol) and (2S,4S)-1-tert-butoxycarbonyl-4-(trifluoromethyl)pyrrolidine-2-carboxylic acid (230 mg, 0.81 mmol) in DCM (4 mL) was added TEA (411 mg, 4.06 mmol) and HATU (463 mg, 1.22 mmol) at 20° C. under N₂. The reaction was stirred at 20° C. for 3 h before it was diluted with H₂O (5 mL) and extracted with EtOAc (3×5 mL). The combined organic layers were washed with brine (5 mL), dried over anhydrous Na₂SO₄, filtered and concentrated under reduced pressure. The resulting residue was purified by prep-TLC (SiO₂, EtOAc) to give the titled compound. LCMS: m/z=451.2 [M+H]⁺.

(2S,4S)—N-(4-methyl-3-(pyridazin-3-yl)phenyl)-4-(trifluoromethyl)pyrrolidine-2-carboxamide hydrochloride: To tert-butyl (2S,4S)-2-((4-methyl-3-(pyridazin-3-yl)phenyl)carbamoyl)-4-(trifluoromethyl)pyrrolidine-1-carboxylate (300 mg, 0.67 mmol) was added HCl/EtOAc (10 mL, 4M) at 25° C. and the reaction mixture was stirred for 1 h. The reaction mixture was concentrated under reduced pressure to give the titled compound. LCMS: m/z=351.2 [M+H]⁺. [0481](2S,4S)—N-(4-methyl-3-(pyridazin-3-yl)phenyl)-1-(pyrimidin-2-yl)-4-(trifluoromethyl)pyrrolidine-2-carboxamide: To a solution of (2S,4S)—N-(4-methyl-3-(pyridazin-3-yl)phenyl)-4-(trifluoromethyl)pyrrolidine-2-carboxamide hydrochloride (200 mg, 0.52 mmol) and 2-chloropyrimidine (71 mg, 0.62 mmol) in DMF (2 mL) was added K₂CO₃ (214 mg, 1.55 mmol) at 20° C. under N₂. The reaction mixture was heated to 80° C. and stirred for 16 h. The reaction mixture was diluted with H₂O (5 mL) and extracted with EtOAc (3×3 mL). The combined organic layers were washed with brine (5 mL), dried over anhydrous Na₂SO₄, filtered and concentrated under reduced pressure. The resulting residue was purified by prep-HPLC (Waters Phenomenex C18 75×30 mm×3 m; mobile phase: A: 10 mM NH₄HCO₃ in water; B: MeCN; B % in A: 15%-45%, 8 min) to give the titled compound. LCMS: m/z=429.2 [M+H]⁺.

### Example 175

### (2R,4R)—N-(3-(5-fluoropyrimidin-2-yl)-4-methylphenyl)-1-(1-methyl-1H-1,2,4-triazol-3-yl)-4-(trifluoromethyl)pyrrolidine-2-carboxamide

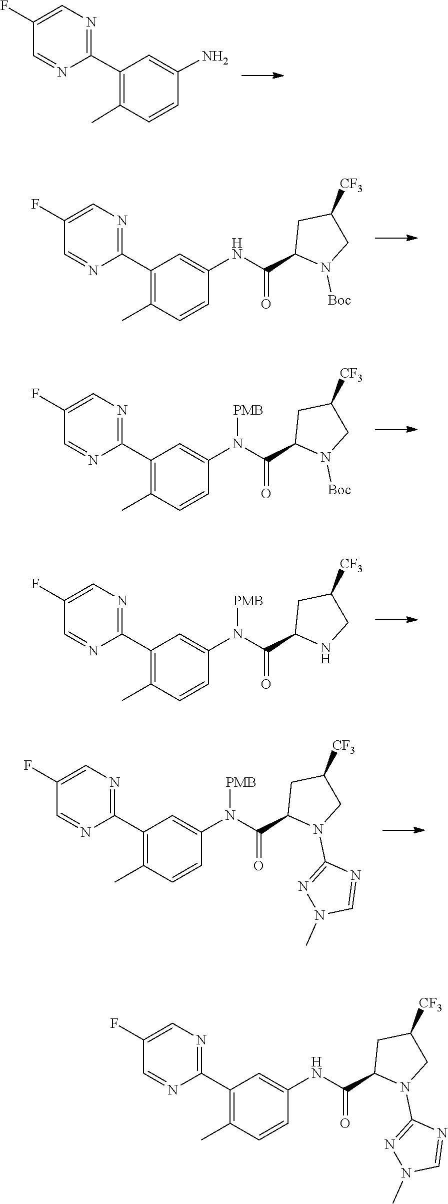

tert-butyl (2R,4R)-2-((3-(5-fluoropyrimidin-2-yl)-4-methylphenyl)carbamoyl)-4-(trifluoromethyl)pyrrolidine-1-carboxylate: To a solution of (2R,4R)-1-(tert-butoxycarbonyl)-4-(trifluoromethyl)pyrrolidine-2-carboxylic acid (350 mg, 1.24 mmol) in DCM (5 mL) was added 3-(5-fluoropyrimidin-2-yl)-4-methyl-aniline (209 mg, 1.03 mmol), HATU (587 mg, 1.54 mmol) and TEA (313 mg, 3.09 mmol) at 20° C. under N₂. The reaction mixture was stirred at 20° C. for 2 h and then diluted with H₂O (5 mL) and extracted with DCM (3×8 mL). The combined organic layers were washed with brine (5 mL), dried over anhydrous Na₂SO₄, filtered and concentrated under reduced pressure. The resulting residue was purified by silica gel column chromatography (PE:EtOAc=5:1 to 1:1) to give the titled compound. LCMS: m/z=369.1 [H]⁺.

tert-butyl (2R,4R)-2-((3-(5-fluoropyrimidin-2-yl)-4-methylphenyl)(4-methoxybenzyl)carbamoyl)-4-(trifluoromethyl)pyrrolidine-1-carboxylate: To a solution of tert-butyl (2R,4R)-2-((3-(5-fluoropyrimidin-2-yl)-4-methylphenyl)carbamoyl)-4-(trifluoromethyl)pyrrolidine-1-carboxylate (300 mg, 0.64 mmol) in DMF (4 mL) at 0° C. under N₂ was added NaH (33 mg, 0.83 mmol, 60% in mineral oil). The mixture was stirred at 0° C. for 0.5 h and then 1-(chloromethyl)-4-methoxy-benzene (110 mg, 0.70 mmol) was added to the solution and stirred at 0° C. for 1 h. The reaction was quenched by addition of aq. sat. NH₄Cl solution (10 mL) and extracted with EtOAc (3×8 mL). The combined organic layers were washed with brine (10 mL), dried over anhydrous Na₂SO₄, filtered and concentrated under reduced pressure. The resulting residue was purified by silica gel column chromatography (PE:EtOAc=10:1 to 1:1) to give the titled compound. LCMS: m/z=589.2 [M+H]⁺.

(2R,4R)—N-(3-(5-fluoropyrimidin-2-yl)-4-methylphenyl)-N-(4-methoxybenzyl)-4-(trifluoromethyl)pyrrolidine-2-carboxamide hydrochloride: A solution of tert-butyl (2R,4R)-2-((3-(5-fluoropyrimidin-2-yl)-4-methylphenyl)(4-methoxybenzyl)carbamoyl)-4-(trifluoromethyl)pyrrolidine-1-carboxylate (230 mg, 0.39 mmol) in HCl/EtOAc (5 mL, 4 M) was stirred at 20° C. for 2 h. The reaction mixture was concentrated under reduced pressure to give the titled compound. LCMS: m/z=489.2 [M+H]⁺.

(2R,4R)—N-(3-(5-fluoropyrimidin-2-yl)-4-methylphenyl)-N-(4-methoxybenzyl)-1-(1-methyl-1H-1,2,4-triazol-3-yl)-4-(trifluoromethyl)pyrrolidine-2-carboxamide: To a solution of (2R,4R)—N-(3-(5-fluoropyrimidin-2-yl)-4-methylphenyl)-N-(4-methoxybenzyl)-4-(trifluoromethyl)pyrrolidine-2-carboxamide hydrochloride (30 mg, 0.06 mmol) in 1,4-dioxane (1 mL) was added 3-bromo-1-methyl-1,2,4-triazole (19 mg, 0.11 mmol), Pd₂(dba)₃·CHCl₃ (5 mg, 0.01 mmol), Xantphos (7 mg, 0.01 mmol) and Cs₂CO₃ (56 mg, 0.17 mmol) at 20° C. under N₂. The mixture was heated to 130° C. and stirred for 12 h. The reaction mixture was diluted with H₂O (5 mL) and extracted with EtOAc (3×3 mL). The combined organic layers were washed with brine (5 mL), dried over anhydrous Na₂SO₄, filtered and concentrated under reduced pressure. The resulting residue was purified by prep-TLC (SiO₂, PE:EtOAc=1:1) to give the titled compound. LCMS: m/z=570.3 [M+H]⁺.

(2R,4R)—N-(3-(5-fluoropyrimidin-2-yl)-4-methylphenyl)-1-(1-methyl-1H-1,2,4-triazol-3-yl)-4-(trifluoromethyl)pyrrolidine-2-carboxamide: To a solution of (2R,4R)—N-(3-(5-fluoropyrimidin-2-yl)-4-methylphenyl)-N-(4-methoxybenzyl)-1-(1-methyl-1H-1,2,4-triazol-3-yl)-4-(trifluoromethyl)pyrrolidine-2-carboxamide (10 mg, 0.018 mmol) in DCM (0.5 mL) was added TFA (462 mg, 4.05 mmol) and TfOH (25 mg, 0.17 mmol) at 20° C. and the reaction was stirred for 2 h. The reaction mixture was diluted with H₂O (5 mL) and extracted with DCM (3×3 mL). The combined organic layers were washed with brine (5 mL), dried over anhydrous Na₂SO₄, filtered and concentrated under reduced pressure. The resulting residue was purified by prep-TLC (SiO₂, EtOAc) to give the titled compound. LCMS: m/z=450.2 [M+H]⁺.

### Examples 176 and 177

### trans-6,6-difluoro-N-(4-methyl-3-(pyridazin-3-yl)phenyl)bicyclo[3.1.0]hexane-3-carboxamide and cis-6,6-difluoro-N-(4-methyl-3-(pyridazin-3-yl)phenyl)bicyclo[3.1.0]hexane-3-carboxamide

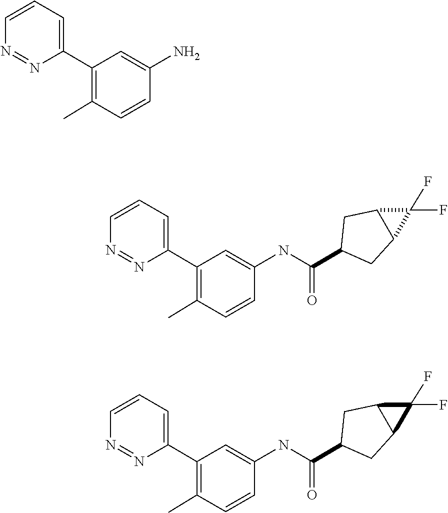

To a mixture of 6,6-difluorobicyclo[3.1.0]hexane-3-carboxylic acid (73 mg, 0.45 mmol) (mixture of cis and trans) in DCM (3 mL) was added 4-methyl-3-(pyridazin-3-yl)aniline (100 mg, 0.54 mmol), TEA (228 mg, 2.25 mmol) and HATU (205 mg, 0.54 mmol) at 25° C. under N₂ and the mixture was stirred at 25° C. for 16 h. Then the reaction mixture was concentrated under reduced pressure and the resulting residue was purified by prep-HPLC (Phenomenex Luna 80×30 mm×3 m; mobile phase: A: 10 mM NH₄HCO₃ in water, B: MeCN; B % in A: 10%-30% over 8 min) and prep-TLC (SiO₂, EtOAc) to give trans-6,6-difluoro-N-(4-methyl-3-(pyridazin-3-yl)phenyl)bicyclo[3.1.0]hexane-3-carboxamide (higher Rf on TLC) Example 176 and cis-6,6-difluoro-N-(4-methyl-3-(pyridazin-3-yl)phenyl)bicyclo[3.1.0]hexane-3-carboxamide (lower Rf on TLC) Example 177. LCMS: 330.1 [M+H]⁺.

### Example 178

### Phenethyl(4-methyl-3-(pyrimidin-2-yl)phenyl)carbamate

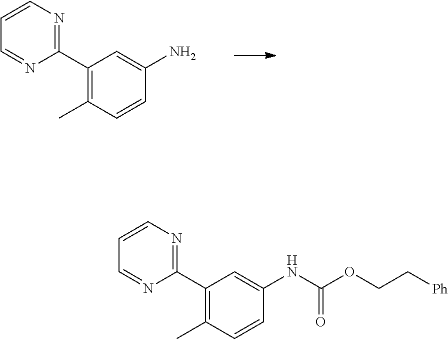 [SMILES: Cc1ccc(N)cc1-c1ncccn1 ; Cc1ccc(NC(=O)OCCc2ccccc2)cc1-c1ncccn1]

To a mixture of CDI (157.58 mg, 0.97 mmol) in DCM (4 mL) was added dropwise a solution of 4-methyl-3-(pyrimidin-2-yl)aniline (150 mg, 0.81 mmol) in DCM (4 mL) at −40° C. under N₂. The mixture was stirred at 25° C. for 12 h. Then 2-phenylethan-1-ol (437 mg, 3.58 mmol) in DCM (5 mL) and TEA (107 mg, 1.07 mmol) were added to the reaction solution at 25° C. under N₂. The mixture was stirred at 25° C. for 4 h. The reaction mixture was concentrated under reduced pressure. The resulting residue was purified by prep-HPLC (Waters Xbridge BEH, C18 100×30 mm×10 μm; mobile phase: A: 10 mM NH₄HCO₃ in water, B: MeCN; B in A: 35%-70% over 8 min) to give the titled compound. LCMS: 334.2 [M+H]⁺.

### Example 179

### cis-N-(4-(fluoromethyl)-3-(5-fluoropyrimidin-2-yl)phenyl)-3-methyl-6-azabicyclo[3.1.1]heptane-6-carboxamide

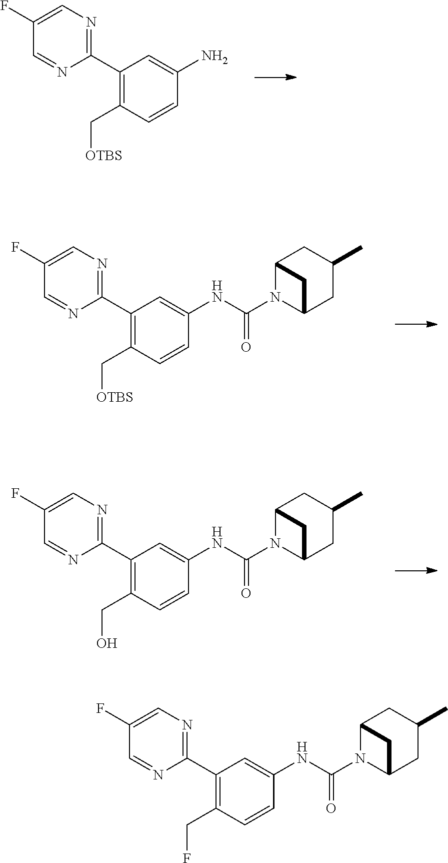

cis-N-(4-(((tert-butyldimethylsilyl)oxy)methyl)-3-(5-fluoropyrimidin-2-yl)phenyl)-3-methyl-6-azabicyclo[3.1.1]heptane-6-carboxamide: To a mixture of triphosgene (134 mg, 0.45 mmol) in THF (5 mL) at 0° C. under N₂ was added TEA (182 mg, 1.80 mmol) and 4-(((tert-butyldimethylsilyl)oxy)methyl)-3-(5-fluoropyrimidin-2-yl)aniline (300 mg, 0.90 mmol). The mixture was warmed to 20° C. and stirred for 1 h. Then cis-3-methyl-6-azabicyclo[3.1.1]heptane (120 mg, 1.08 mmol) and TEA (182 mg, 1.80 mmol) were added and the reaction solution was stirred at 20° C. for 2 h. The reaction mixture was diluted with H₂O (5 mL) and extracted with EtOAc (3×5 mL). The combined organic layers were washed with brine (5 mL), dried over anhydrous Na₂SO₄, filtered and concentrated under reduced pressure. The resulting residue was purified by silica gel column chromatography (PE:EtOAc=10:1 to 1:1) to give the titled compound. LCMS: m/z=493.2 [M+Na]⁺.

cis-N-(3-(5-fluoropyrimidin-2-yl)-4-(hydroxymethyl)phenyl)-3-methyl-6-azabicyclo[3.1.1]heptane-6-carboxamide: To a solution of cis-N-(4-(((tert-butyldimethylsilyl)oxy)methyl)-3-(5-fluoropyrimidin-2-yl)phenyl)-3-methyl-6-azabicyclo[3.1.1]heptane-6-carboxamide (300 mg, 0.68 mmol) in THF (5 mL) at 0° C. was added TBAF (1.27 mL, 1 M in THF). The mixture was warmed to 20° C. and stirred for 3 h. The reaction mixture was diluted with H₂O (10 mL) and extracted with EtOAc (3×5 mL). The combined organic layers were washed with brine (5 mL), dried over anhydrous Na₂SO₄, filtered and concentrated under reduced pressure. The resulting residue was purified by silica gel column chromatography (PE:EtOAc=10:1 to 0:1) to give the titled compound.

cis-N-(4-(fluoromethyl)-3-(5-fluoropyrimidin-2-yl)phenyl)-3-methyl-6-azabicyclo[3.1.1]heptane-6-carboxamide: To a mixture of cis-N-(3-(5-fluoropyrimidin-2-yl)-4-(hydroxymethyl)phenyl)-3-methyl-6-azabicyclo[3.1.1]heptane-6-carboxamide (150 mg, 0.42 mmol) in DCM (3 mL) was added N,N-diethylethanamine trihydrofluoride (136 mg, 0.84 mmol) and (difluoro-)⁴-sulfanylidene)-diethyl-ammonium tetrafluoroborate (193 mg, 0.84 mmol) at −78° C. under N₂. The reaction was warmed to 20° C. for 2 h. The reaction mixture was diluted with H₂O (10 mL) and extracted with EtOAc (3×5 mL). The combined organic layers were washed with brine (3×5 mL), dried over anhydrous Na₂SO₄, filtered and concentrated under reduced pressure. The resulting residue was purified by prep-HPLC (Waters Xbridge Prep OBD C18 150×40 mm×10 m; mobile phase: A: NH₄HCO₃ in water; B: MeCN, B % in A: 25%-55%, 8 min) to give the titled compound.

LCMS: m/z=359.2 [M+H]⁺.

### Example 180

### N-(4-methyl-3-(6-methyl-1,2,4-triazin-3-yl)phenyl)-6-azabicyclo[3.1.1]heptane-6-carboxamide

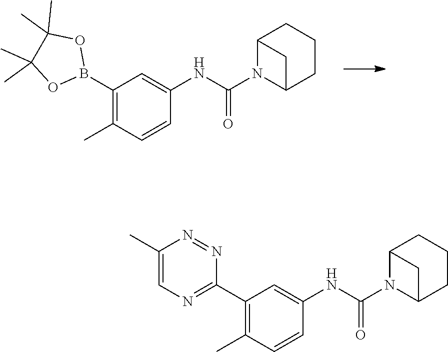 [SMILES: Cc1ccc(NC(=O)N2C3CCCC2C3)cc1B1OC(C)(C)C(C)(C)O1 ; Cc1cnc(-c2cc(NC(=O)N3C4CCCC3C4)ccc2C)nn1]

To a solution of 3-chloro-6-methyl-1,2,4-triazine (40 mg, 0.31 mmol) and N-(4-methyl-3-(4,4,5,5-tetramethyl-1,3,2-dioxaborolan-2-yl)phenyl)-6-azabicyclo[3.1.1]heptane-6-carboxamide (132 mg, 0.37 mmol) in 1,4-dioxane (2 mL) and H₂O (0.2 mL) was added K₂CO₃ (107 mg, 0.77 mmol) and Pd(dppf)Cl₂ (23 mg, 0.03 mmol) at 20° C. under N₂. The mixture was heated to 110° C. and stirred for 12 h. The reaction mixture was filtered through a celite pad and the filtrate was concentrated under reduced pressure. The resulting residue was purified by prep-HPLC (Waters Xbridge Prep OBD C18 150×40 mm×10 m; mobile phase: A: 10 mM NH₄HCO₃ in water, B: MeCN; B % in A: 25%-55%, 8 min) to give the titled compound. LCMS: m/z=324.0 [M+H]⁺.

### Example 181

### N-(2-fluoro-4-methyl-5-(1,3,5-triazin-2-yl)phenyl)-6-azabicyclo[3.1.1]heptane-6-carboxamide

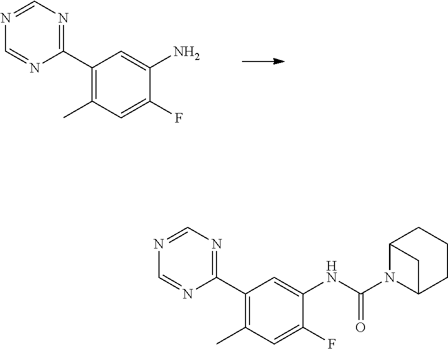 [SMILES: Cc1cc(F)c(N)cc1-c1ncncn1 ; Cc1cc(F)c(NC(=O)N2C3CCCC2C3)cc1-c1ncncn1]

To a mixture of triphosgene (51 mg, 0.17 mmol) in THF (2 mL) at 0° C. was added a solution of 2-fluoro-4-methyl-5-(1,3,5-triazin-2-yl)aniline (70 mg, 0.34 mmol) in THF (2 mL) and the solution was warmed to 20° C. and stirred for 1 h. Then 6-azabicyclo[3.1.1]heptane (67 mg, 0.69 mmol) and TEA (104 mg, 1.03 mmol) were added and the reaction mixture was stirred an additional 1 h. The reaction mixture was diluted with H₂O (3 mL) and extracted with EtOAc (3×5 mL). The combined organic layers were washed with brine (5 mL), dried over anhydrous Na₂SO₄, filtered and concentrated under reduced pressure. The resulting residue was purified by prep-HPLC (Waters Xbridge BEH C18 100×30 mm×m; mobile phase: A: 10 mM NH₄HCO₃ in water, B: MeCN; B % in A: 25%-55%, 8 min) and further purified by prep-TLC (SiO₂, PE:EtOAc=1:2) to give the titled compound. LCMS: m/z=328.2 [M+H]⁺.

### Example 182

### N-(2-fluoro-4-methyl-5-(pyridazin-3-yl)phenyl)-5-methyl-2,3-dihydropyrido[2,3-f][1,4]oxazepine-4(5H)-carboxamide

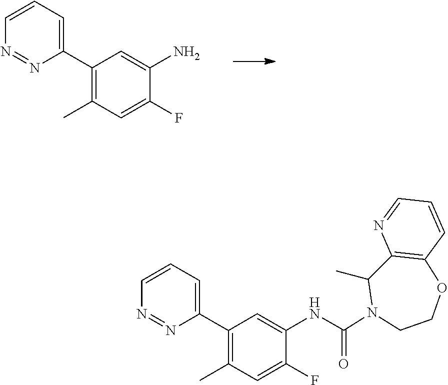 [SMILES: Cc1cc(F)c(N)cc1-c1cccnn1 ; Cc1cc(F)c(NC(=O)N2CCOc3cccnc3C2C)cc1-c1cccnn1]

To a solution of triphosgene (37 mg, 0.12 mmol) in THF (2 mL) was added TEA (75 mg, 0.74 mmol) and 2-fluoro-4-methyl-5-(pyridazin-3-yl)aniline (50 mg, 0.25 mmol) at 0° C. under N₂. The reaction mixture was stirred at 0° C. for 1 h. Then 5-methyl-2,3,4,5-tetrahydropyrido[2,3-f][1,4]oxazepine (54 mg, 0.33 mmol) was added and the reaction mixture was warmed to 25° C. and stirred for 12 h. Water was added and the mixture was concentrated in vacuo. The resulting residue was purified by prep-HPLC (Waters Xbridge BEH C18 100×30 mm×10 μm; mobile phase: A: 10 mM NH₄HCO₃ in water, B: MeCN; B % in A: 25%-55% over 8 min) to give the titled compound. LCMS: m/z=394.2 [M+H].

### Example 183

### cis-N-(2-fluoro-5-(5-fluoropyrimidin-2-yl)-4-methylphenyl)-1-(2-hydroxypropan-2-yl)-3-methyl-6-azabicyclo[3.1.1]heptane-6-carboxamide

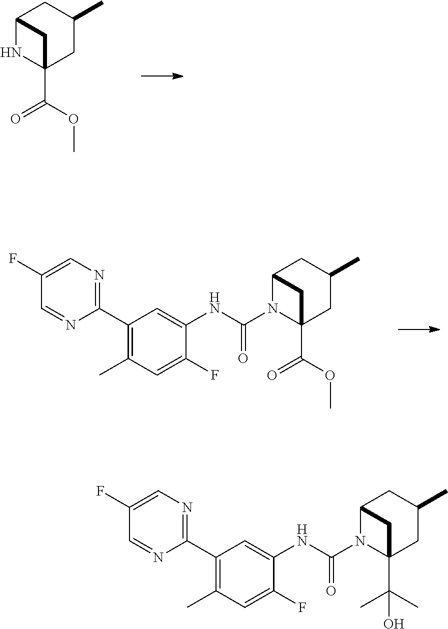

methyl cis-6-((2-fluoro-5-(5-fluoropyrimidin-2-yl)-4-methylphenyl)carbamoyl)-3-methyl-6-azabicyclo[3.1.1]heptane-1-carboxylate: To a solution of triphosgene (33 mg, 0.1 mmol) in THF (2 mL) at 0° C. under N₂ was added 2-fluoro-5-(5-fluoropyrimidin-2-yl)-4-methylaniline (50 mg, 0.2 mmol) and TEA (46 mg, 0.5 mmol). The mixture was warmed to 25° C. and stirred for 1 h. Then methyl cis-3-methyl-6-azabicyclo[3.1.1]heptane-1-carboxylate (489 mg, 1.42 mmol, 49% purity) and TEA (204 mg, 2.02 mmol) were added to the above mixture and stirred for 1 h. The reaction mixture was diluted with H₂O (3 mL) and extracted with EtOAc (3×5 mL). The combined organic layers were washed with brine (5 mL), dried over anhydrous Na₂SO₄, filtered and concentrated under reduced pressure. The resulting residue was purified by prep-TLC (SiO₂, PE:EtOAc=3:1) to give the titled compound. LCMS: m/z=417.1 [M+H]⁺.

cis-N-(2-fluoro-5-(5-fluoropyrimidin-2-yl)-4-methylphenyl)-1-(2-hydroxypropan-2-yl)-3-methyl-6-azabicyclo[3.1.1]heptane-6-carboxamide: To a solution of methyl cis-6-((2-fluoro-5-(5-fluoropyrimidin-2-yl)-4-methylphenyl)carbamoyl)-3-methyl-6-azabicyclo[3.1.1]heptane-1-carboxylate (50 mg, 0.12 mmol) in THF (2 mL) at 0° C. under N₂ was added MeMgBr (0.2 mL, 3M in THF, 0.6 mmol). The mixture was warmed to 25° C. and stirred for 1 h. The reaction was quenched by addition of aq. sat. NH₄Cl (2 mL) and the mixture was extracted with EtOAc (3×3 mL). The combined organic layers were washed with brine (3 mL), dried over anhydrous Na₂SO₄, filtered and concentrated under reduced pressure. The resulting residue was purified by prep-TLC (SiO₂, PE:EtOAc=2:1) to give the titled compound. LCMS: m/z=417.2 [M+H]⁺.

### Example 184

### cis-3-methyl-N-(4-methyl-3-(5-(trifluoromethyl)-1,2,4-triazin-3-yl)phenyl)-6-azabicyclo[3.1.1]heptane-6-carboxamide

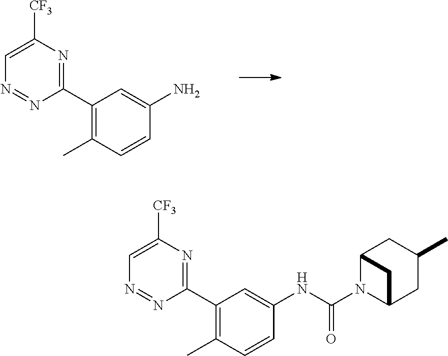

To a solution of triphosgene (26 mg, 0.089 mmol) in THF (1 mL) at 0° C. under N₂ was added TEA (60 mg, 0.6 mmol) and 4-methyl-3-[5-(trifluoromethyl)-1,2,4-triazin-3-yl]aniline (50 mg, 0.20 mmol) and the reaction was stirred for 1 h. Then cis-3-methyl-6-azabicyclo[3.1.1]heptane (55 mg, 0.49 mmol) and TEA (60 mg, 0.59 mmol) were added to the solution and the reaction mixture was warmed to 25° C. and stirred for 2 h. Water was added and the mixture was concentrated in vacuo. The crude product was purified by prep-TLC and prep-HPLC (Waters Xbridge BEH C18 100×30 mm×10 m; mobile phase: mobile phase: A: 10 mM NH₄HCO₃ in water, B: MeCN; B % in A: 35%-65%, 8 min) to give the titled compound. LCMS: m/z=392.2 [M+H]⁺.

### Example 185

### cis-N-(3-(5-(fluoromethoxy)pyridazin-3-yl)-4-methylphenyl)-3-methyl-6-azabicyclo[3.1.1]heptane-6-carboxamide

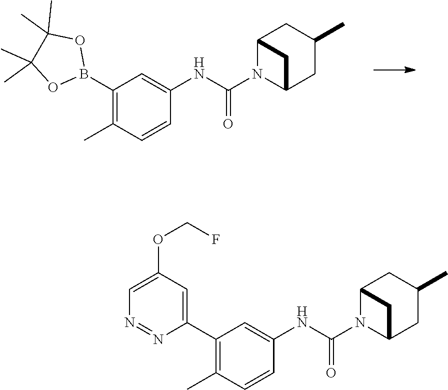

To a solution of cis-3-methyl-N-[4-methyl-3-(4,4,5,5-tetramethyl-1,3,2-dioxaborolan-2-yl)phenyl]-6-azabicyclo[3.1.1]heptane-6-carboxamide (50 mg, 0.14 mmol) and 3-chloro-5-(fluoromethoxy)pyridazine (32 mg, 0.20 mmol) in 1,4-dioxane (2 mL) and H₂O (0.2 mL) was added K₂CO₃ (46 mg, 0.34 mmol) and Pd(dppf)Cl₂ (9 mg, 0.013 mmol) at 20° C. under N₂. The mixture was heated to 100° C. and stirred for 12 h. The reaction mixture was filtered through a celite pad and the filtrate was concentrated under reduced pressure. The resulting residue was purified by prep-HPLC (Phenomenex C 18 150×40 mm×10 m; mobile phase A: 10 mM NH₄HCO₃ in water, B: MeCN; B % in A: B %:25%-55%, 8 min) to give the titled compound. LCMS: m/z=371.1 [M+H]⁺.

### Examples 186 and 187

### (1S,3R,5R)-1-(1-ethoxyethyl)-N-(3-(5-fluoropyrimidin-2-yl)-4-methylphenyl)-3-methyl-6-azabicyclo[3.1.1]heptane-6-carboxamide and (1R,3S,5S)-1-(1-ethoxyethyl)-N-(3-(5-fluoropyrimidin-2-yl)-4-methylphenyl)-3-methyl-6-azabicyclo[3.1.1]heptane-6-carboxamide

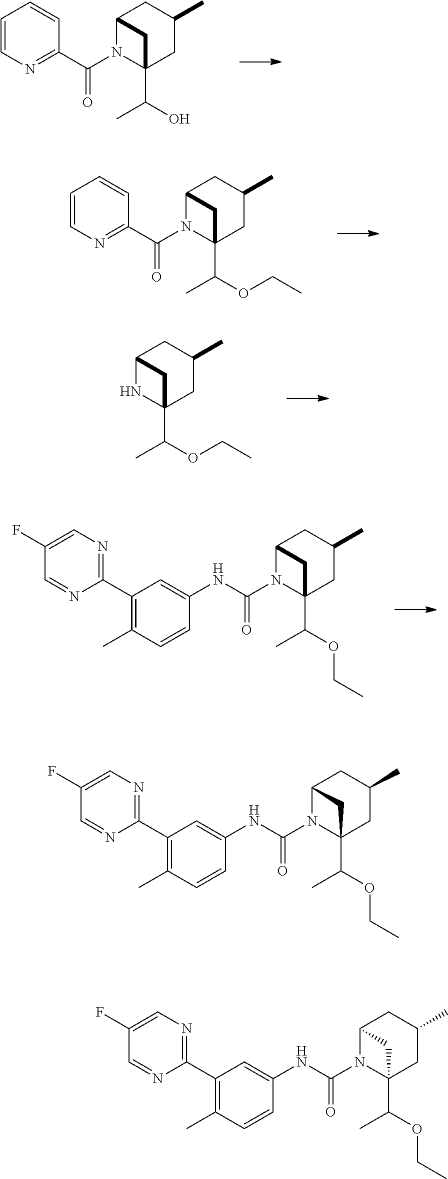

(cis-1-(1-ethoxyethyl)-3-methyl-6-azabicyclo[3.1.1]heptan-6-yl)(pyridin-2-yl)methanone: To a solution of Intermediate 61, (cis-1-(1-hydroxyethyl)-3-methyl-6-azabicyclo[3.1.1]heptan-6-yl)(pyridin-2-yl)methanone (400 mg, 1.54 mmol), in toluene (30 mL) was added Ag₂O (2.49 g, 10.76 mmol) and EtI (1.68 g, 10.76 mmol) at 20° C. under N₂ in a sealed tube. The mixture was stirred at 100° C. for 16 h. The mixture was filtered and the filtrate was concentrated under reduced pressure. The resulting residue was purified by silica gel column chromatography (PE:MTBE=5:1 to 1:1) to give the titled compound.

cis-1-(1-ethoxyethyl)-3-methyl-6-azabicyclo[3.1.1]heptane: To a mixture of (cis-1-(1-ethoxyethyl)-3-methyl-6-azabicyclo[3.1.1]heptan-6-yl)(pyridin-2-yl)methanone (120 mg, 0.42 mmol) in THF (10 mL) was added MeMgBr (0.70 mL, 3 M in Et₂O) at 0° C. under N₂. The mixture was stirred at 20° C. for 7 h. The reaction mixture was quenched by addition of sat. NH₄Cl (1 mL). The mixture was concentrated under reduced pressure and the residue was slurried with DCM (20 mL), filtered and the filtrate was concentrated under reduce pressure to give the titled compound.

cis-1-(1-ethoxyethyl)-N-(3-(5-fluoropyrimidin-2-yl)-4-methylphenyl)-3-methyl-6-azabicyclo[3.1.1]heptane-6-carboxamide: To a mixture of triphosgene (37 mg, 0.12 mmol) in THF (10 mL) was added a mixture of TEA (373 mg, 3.69 mmol) and 3-(5-fluoropyrimidin-2-yl)-4-methylaniline (250 mg, 1.23 mmol) in THF (10 mL) at 0° C. under N₂. The mixture was stirred at 0° C. for 1 h. Then a solution of TEA (132 mg, 0.74 mmol) and cis-1-(1-ethoxyethyl)-3-methyl-6-azabicyclo[3.1.1]heptane (120 mg, 0.65 mmol) in THF (6 mL) was added to the reaction solution. The mixture was stirred at 20° C. for 2 h. The reaction mixture was diluted with H₂O (5 mL) and extracted with EtOAc (3×5 mL). The combined organic layers were washed with brine (5 mL), dried over anhydrous Na₂SO₄, filtered and concentrated under reduced pressure. The resulting residue was purified by prep-HPLC (column: Waters Xbridge BEH, C18 100×30 mm×10 μm; mobile phase: A: 10 mM NH₄HCO₃ in water, B: MeCN; B in A: 48%-78% over 8 min) to give the titled compound as a mixture of enantiomers. LCMS: m/z=413.2 [M+H]⁺.

(1S,3R,5R)-1-(1-ethoxyethyl)-N-(3-(5-fluoropyrimidin-2-yl)-4-methylphenyl)-3-methyl-6-azabicyclo[3.1.1]heptane-6-carboxamide and (1R,3S,5S)-1-(1-ethoxyethyl)-N-(3-(5-fluoropyrimidin-2-yl)-4-methylphenyl)-3-methyl-6-azabicyclo[3.1.1]heptane-6-carboxamide: The mixture of enantiomers was separated by SFC (column: ChiralPak IH, 250 mm×25 mm, 10 m; Mobile phase: A: CO₂, B: 0.1% NH₃H₂O in EtOH; B % in A: 20%-20%, 14 min; Flow rate: 70 g/min; Wavelength: 150 nm; Column temperature: 35° C.; System back pressure: 120 bar) to give cis-(1-(1-ethoxyethyl)-N-(3-(5-fluoropyrimidin-2-yl)-4-methylphenyl)-3-methyl-6-azabicyclo[3.1.1]heptane-6-carboxamide (peak 1 in SFC) Example 186. LCMS: m/z=413.2 [M+H]⁺ and cis-1-(1-ethoxyethyl)-N-(3-(5-fluoropyrimidin-2-yl)-4-methylphenyl)-3-methyl-6-azabicyclo[3.1.1]heptane-6-carboxamide (peak 2 in SFC) Example 187. LCMS: m/z=413.2 [M+H]⁺.

### Examples 188 and 189

### (1S,3R,5R)-1-(1-ethoxyethyl)-N-(3-(5-fluoropyrimidin-2-yl)-4-methylphenyl)-3-methyl-6-azabicyclo[3.1.1]heptane-6-carboxamide and (1R,3S,5S)-1-(1-ethoxyethyl)-N-(3-(5-fluoropyrimidin-2-yl)-4-methylphenyl)-3-methyl-6-azabicyclo[3.1.1]heptane-6-carboxamide

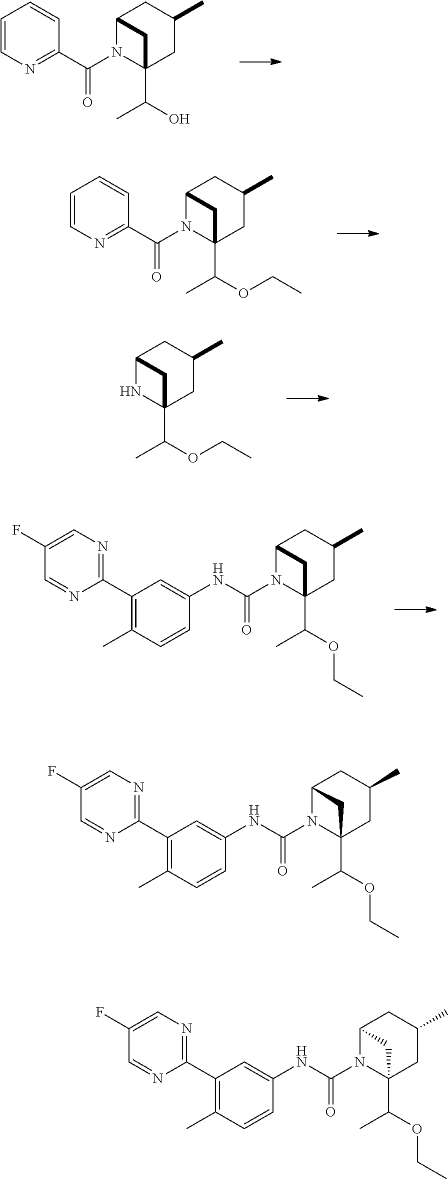

(cis-1-(1-ethoxyethyl)-3-methyl-6-azabicyclo[3.1.1]heptan-6-yl)(255yridine-2-yl)methanone: To a solution of Intermediate 62, (cis-1-(1-hydroxyethyl)-3-methyl-6-azabicyclo[3.1.1]heptan-6-yl)(255yridine-2-yl)methanone (280 mg, 1.08 mmol), in DMF (5 mL) was added NaH (65 mg, 1.61 mmol, 60% in mineral oil) at 0° C. under N₂. The mixture was stirred at 0° C. for 0.5 h before adding EtI (336 mg, 2.15 mmol) at 0° C. The mixture was stirred at 20° C. for 12 h. The mixture was quenched by addition of sat. NH₄Cl (30 mL) and extracted with EtOAc (3×10 mL). The combined organic layers were washed with brine (10 mL), dried over anhydrous Na₂SO₄, filtered and concentrated under reduced pressure. The resulting residue was purified by silica gel column chromatography (PE:EtOAc=5:1 to 1:1) to give the titled compound. LCMS: m/z=289.2 [M+H]⁺.

Cis-1-(1-ethoxyethyl)-3-methyl-6-azabicyclo[3.1.1]heptane: To a mixture of (cis-1-(1-ethoxyethyl)-3-methyl-6-azabicyclo[3.1.1]heptan-6-yl)(255yridine-2-yl)methanone (150 mg, 0.52 mmol) in THF (5 mL) was added MeMgBr (0.87 mL, 3 M in Et₂O) at 0° C. under N₂. The mixture was stirred at 20° C. for 14 h. The reaction mixture was quenched by addition of sat. NH₄Cl (1 mL) and concentrated under reduced pressure. The resulting residue was slurried with DCM (10 mL), filtered and the filtrate was concentrated under reduced pressure to give the titled compound.

Cis-1-(1-ethoxyethyl)-N-(3-(5-fluoropyrimidin-2-yl)-4-methylphenyl)-3-methyl-6-azabicyclo[3.1.1]heptane-6-carboxamide: To a mixture of triphosgene (37 mg, 0.12 mmol) in THF (3 mL) was added a mixture of TEA (75 mg, 0.74 mmol) and 3-(5-fluoropyrimidin-2-yl)-4-methylaniline (50 mg, 0.25 mmol) in THF (2 mL) at 0° C. under N₂. The mixture was stirred at 0° C. for 1 h. Then a solution of TEA (75 mg, 0.74 mmol) and cis-1-(1-ethoxyethyl)-3-methyl-6-azabicyclo[3.1.1]heptane (90 mg, 0.49 mmol) in THF (2 mL) was added at 0° C. The mixture was stirred at 20° C. for 2 h. The reaction mixture was diluted with H₂O (5 mL) and extracted with EtOAc (3×5 mL). The combined organic layers were washed with brine (5 mL), dried over anhydrous Na₂SO₄, filtered and concentrated under reduced pressure. The resulting residue was purified by prep-TLC (SiO₂, PE:EtOAc=1:2) to give the titled compound as a mixture of enantiomers. LCMS: m/z=413.2 [M+H]⁺.

(1S,3R,5R)-1-(1-ethoxyethyl)-N-(3-(5-fluoropyrimidin-2-yl)-4-methylphenyl)-3-methyl-6-azabicyclo[3.1.1]heptane-6-carboxamide and (1R,3S,5S)-1-(1-ethoxyethyl)-N-(3-(5-fluoropyrimidin-2-yl)-4-methylphenyl)-3-methyl-6-azabicyclo[3.1.1]heptane-6-carboxamide: The mixture of enantiomers was separated by SFC (ChiralPak IH, 250 mm×25 mm, 10 m; Mobile phase: A: CO₂, B: 0.1% NH₃H₂O in EtOH; B % in A: 30%-30%, 12 min; Flow rate: 70 g/min; Wavelength: 150 nm; Column temperature: 35° C.; System back pressure: 120 bar) to give cis-1-(1-ethoxyethyl)-N-(3-(5-fluoropyrimidin-2-yl)-4-methylphenyl)-3-methyl-6-azabicyclo[3.1.1]heptane-6-carboxamide (peak 1 in SFC) Example 188. LCMS: m/z=413.2 [M+H]⁺ and cis-1-(1-ethoxyethyl)-N-(3-(5-fluoropyrimidin-2-yl)-4-methylphenyl)-3-methyl-6-azabicyclo[3.1.1]heptane-6-carboxamide (peak 2 in SFC) Example 189. LCMS: m/z=413.2 [M+H]⁺.

### Example 190

### cis-N-(3-(5-fluoropyrimidin-2-yl)-4-methylphenyl)-1-(1-methoxyethyl)-3-methyl-6-azabicyclo[3.1.1]heptane-6-carboxamide

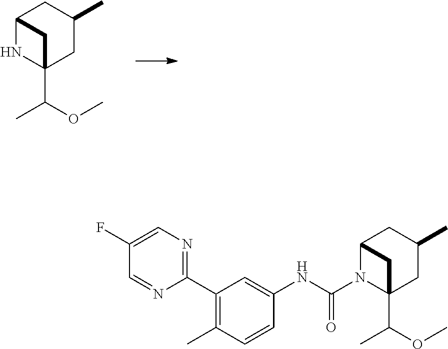

To a mixture of triphosgene (37 mg, 0.12 mmol) in THF (3 mL) was added a mixture of TEA (75 mg, 0.74 mmol) and 3-(5-fluoropyrimidin-2-yl)-4-methyl-aniline (50 mg, 0.24 mmol) in THF (3 mL) at 0° C. under N₂. The mixture was stirred at 0° C. for 1 h before a solution of TEA (75 mg, 0.74 mmol) and Intermediate 60, cis-1-(−1-methoxyethyl)-3-methyl-6-azabicyclo[3.1.1]heptane (125 mg, 0.74 mmol), in THF (3 mL) was added at 0° C. The mixture was stirred at 20° C. for 2 h. The reaction mixture was diluted with H₂O (5 mL) and extracted with EtOAc (3×5 mL). The combined organic layers were washed with brine (5 mL), dried over anhydrous Na₂SO₄, filtered and concentrated under reduced pressure. The resulting residue was purified by prep-HPLC (Waters Xbridge Prep OBD C18 100×30 mm×10 m; mobile phase: A: 10 mM NH₄HCO₃ in water, B: MeCN; B % in A: 50%-70%, 8 min) to provide the titled compound as a mixture of enantiomers. LCMS: m/z=399.2 [M+H]⁺.

### Examples 191, 192, 193, and 194

### (1R,3S,5S)—N-(3-(5-fluoropyrimidin-2-yl)-4-methylphenyl)-1-(1-methoxyethyl)-3-methyl-6-azabicyclo[3.1.1]heptane-6-carboxamide and (1S,3R,5R)—N-(3-(5-fluoropyrimidin-2-yl)-4-methylphenyl)-1-(1-methoxyethyl)-3-methyl-6-azabicyclo[3.1.1]heptane-6-carboxamide and (1S,3R,5R)—N-(3-(5-fluoropyrimidin-2-yl)-4-methylphenyl)-1-(1-hydroxyethyl)-3-methyl-6-azabicyclo[3.1.1]heptane-6-carboxamide and (1R,3S,5S)—N-(3-(5-fluoropyrimidin-2-yl)-4-methylphenyl)-1-(1-hydroxyethyl)-3-methyl-6-azabicyclo[3.1.1]heptane-6-carboxamide

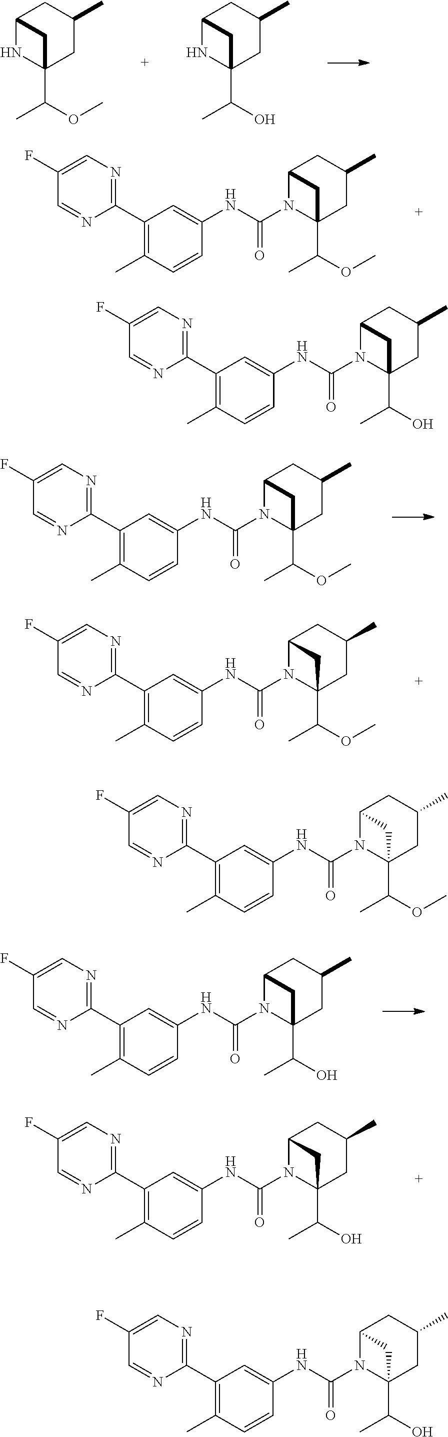

To a mixture of triphosgene (73 mg, 0.25 mmol) in THF (3 mL) was added a mixture of TEA (149 mg, 1.48 mmol) and 3-(5-fluoropyrimidin-2-yl)-4-methyl-aniline (100 mg, 0.49 mmol) in THF (3 mL) at 0° C. under N₂. The mixture was stirred at 0° C. for 1 h before a solution of TEA (100 mg, 0.98 mmol) and Intermediate 59, cis-1-(1-methoxyethyl)-3-methyl-6-azabicyclo[3.1.1]heptane and 1-(cis-3-methyl-6-azabicyclo[3.1.1]heptan-1-yl)ethan-1-ol (250 mg, 1.48 mmol), in THF (3 mL) was added at 0° C. The mixture was stirred at 20° C. for 2 h. The reaction mixture was diluted with H₂O (7 mL) and extracted with EtOAc (3×5 mL). The combined organic layers were washed with brine (5 mL), dried over anhydrous Na₂SO₄, filtered and concentrated under reduced pressure. The resulting residue was purified by prep-HPLC (Waters Xbridge Prep OBD C18 150×40 mm×10 m; mobile phase: A: 10 mM NH₄HCO₃ in water, B: MeCN; B % in A: 40%-70%, 8 min) to give two peaks. The first eluting peak was further purified by prep-TLC (SiO₂, PE:EtOAc=1:1) to provide cis-N-(3-(5-fluoropyrimidin-2-yl)-4-methylphenyl)-1-(1-hydroxyethyl)-3-methyl-6-azabicyclo[3.1.1]heptane-6-carboxamide as a mixture of enantiomers. LCMS: m/z=383.2 [M−H]⁻. The second eluting peak was further purified by prep-TLC (SiO₂, PE:EtOAc=1:1) to give cis-N-(3-(5-fluoropyrimidin-2-yl)-4-methylphenyl)-1-(1-methoxyethyl)-3-methyl-6-azabicyclo[3.1.1]heptane-6-carboxamide as a mixture of enantiomers. LCMS: m/z=399.2 [M+H]⁺.

The mixture of enantiomers of cis-N-(3-(5-fluoropyrimidin-2-yl)-4-methylphenyl)-1-(1-methoxyethyl)-3-methyl-6-azabicyclo[3.1.1]heptane-6-carboxamide was separated by SFC (DAICEL CHIRALCEL OZ, 250 mm×30 mm, 10 mm; Mobile phase: A: CO₂, B: 0.1% NH₃H₂O in MeOH; B % in A: 40%-40%, Flow rate: 70 g/min; Wavelength: 220 nm; Column temperature: 40° C.; System back pressure: 100 bar) to provide two impure peaks. The first eluting peak was further purified by prep-HPLC (Waters Xbridge Prep OBD C18 150×40 mm×10 m; mobile phase: A: 10 mM NH₄HCO₃ in water, B: MeCN; B % in A: 40%-70%, 8 min) to give cis-N-(3-(5-fluoropyrimidin-2-yl)-4-methylphenyl)-1-(1-methoxyethyl)-3-methyl-6-azabicyclo[3.1.1]heptane-6-carboxamide (peak 1 in SFC) Example 191. LCMS: m/z=399.2 [M+H]⁺. The second eluting peak was further purified by prep-HPLC (Waters Xbridge Prep OBD C18 150×40 mm×10 m; mobile phase: A: 10 mM NH₄HCO₃ in water, B: MeCN; B % in A: 50%-80%, 8 min) to give cis-N-(3-(5-fluoropyrimidin-2-yl)-4-methylphenyl)-1-(−1-methoxyethyl)-3-methyl-6-azabicyclo[3.1.1]heptane-6-carboxamide (peak 2 in SFC) Example 192. LCMS: m/z=399.2 [M+H]⁺.

The mixture of enantiomers of cis-N-(3-(5-fluoropyrimidin-2-yl)-4-methylphenyl)-1-(1-hydroxyethyl)-3-methyl-6-azabicyclo[3.1.1]heptane-6-carboxamide was separated by SFC (ChiralPak IH, 250 mm×30 mm, 10 m; Mobile phase: A: CO₂, B: 0.1% NH₃H₂O in EtOH; B % in A: 45%-45%, 10 min; Flow rate: 75 g/min; Wavelength: 220 nm; Column temperature: 35° C.; System back pressure: 120 bar) to give cis-N-(3-(5-fluoropyrimidin-2-yl)-4-methylphenyl)-1-(1-hydroxyethyl)-3-methyl-6-azabicyclo[3.1.1]heptane-6-carboxamide (peak 1 in SFC) Example 193. LCMS: m/z=383.2 [M−H]⁻ and cis-N-(3-(5-fluoropyrimidin-2-yl)-4-methylphenyl)-1-(1-hydroxyethyl)-3-methyl-6-azabicyclo[3.1.1]heptane-6-carboxamide (peak 2 in SFC) Example 194. LCMS: m/z=383.2 [M−H]⁻.

### Examples 195 and 196

### (1S,2S,4R)—N-[3-(5-fluoropyrimidin-2-yl)-4-methylphenyl]-2-methyl-7-azabicyclo[2.2.1]heptane-7-carboxamide and (1R,2R,4S)—N-[3-(5-fluoropyrimidin-2-yl)-4-methylphenyl]-2-methyl-7-azabicyclo[2.2.1]heptane-7-carboxamide

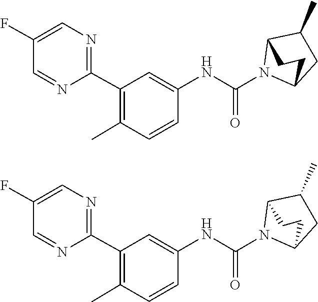 [SMILES: Cc1ccc(NC(=O)N2[C@@H]3CC[C@H]2[C@@H](C)C3)cc1-c1ncc(F)cn1 ; Cc1ccc(NC(=O)N2[C@H]3CC[C@@H]2[C@H](C)C3)cc1-c1ncc(F)cn1]

The mixture of enantiomers was separated by SFC (ChiralPak IH, 250 mm×30 mm, 10 μm; mobile phase: A: CO₂, B: 0.1% NH₃H₂O in EtOH; B % in A: 50%-50%, 6 min; Flow rate: 70 g/min; Wavelength: 220 nm; Column temperature: 35° C.; System back pressure: 120 bar) to give cis-N-(3-(5-fluoropyrimidin-2-yl)-4-methylphenyl)-2-methyl-7-azabicyclo[2.2.1]heptane-7-carboxamide (peak 1 in SFC) Example 195. LCMS: m/z=341.2 [M+H]⁺ and cis-N-(3-(5-fluoropyrimidin-2-yl)-4-methylphenyl)-2-methyl-7-azabicyclo[2.2.1]heptane-7-carboxamide (peak 2 in SFC) Example 196. LCMS: m/z=341.1 [M+H]⁺.

### Examples 197 and 198

### (1R,2S,4S)—N-[3-(5-fluoropyrimidin-2-yl)-4-methylphenyl]-2-methyl-7-azabicyclo[2.2.1]heptane-7-carboxamide and (1S,2R,4R)—N-[3-(5-fluoropyrimidin-2-yl)-4-methylphenyl]-2-methyl-7-azabicyclo[2.2.1]heptane-7-carboxamide

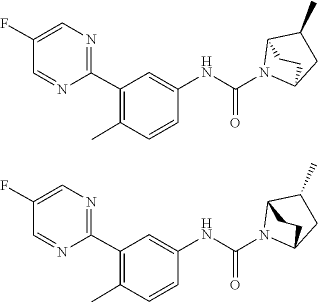 [SMILES: Cc1ccc(NC(=O)N2[C@H]3CC[C@@H]2[C@@H](C)C3)cc1-c1ncc(F)cn1 ; Cc1ccc(NC(=O)N2[C@@H]3CC[C@H]2[C@H](C)C3)cc1-c1ncc(F)cn1]

The mixture of enantiomers was separated by SFC (REGIS (s,s) WHELK-01 250×25 mm×10 m; mobile phase: A: CO₂, B: 0.1% NH₃H₂O in MeOH; B % in A: 45%, Flow rate: 70 g/min; Wavelength: 220 nm; Column temperature: 35° C.; System back pressure: 120 bar) to give trans-N-[3-(5-fluoropyrimidin-2-yl)-4-methylphenyl]-2-methyl-7-azabicyclo[2.2.1]heptane-7-carboxamide (peak 1 in SFC) Example 197. LCMS: m/z=341.1 [M+H]⁺ and trans-N-[3-(5-fluoropyrimidin-2-yl)-4-methylphenyl]-2-methyl-7-azabicyclo[2.2.1]heptane-7-carboxamide (peak 2 in SFC) Example 198. LCMS: m/z=341.1 [M+H]⁺.

### Examples 199 and 200

### (1S,3S,5R)—N-(2-fluoro-4-methyl-5-pyrimidin-2-ylphenyl)-1-(methoxymethyl)-3-methyl-6-azabicyclo[3.1.1]heptane-6-carboxamide and (1R,3R,5S)—N-(2-fluoro-4-methyl-5-(pyrimidin-2-yl)phenyl)-1-(methoxymethyl)-3-methyl-6-azabicyclo[3.1.1]heptane-6-carboxamide

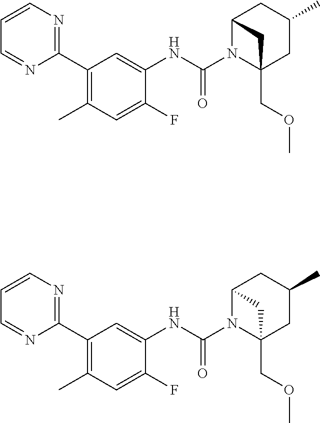 [SMILES: COC[C@]12C[C@@H](C)C[C@H](C1)N2C(=O)Nc1cc(-c2ncccn2)c(C)cc1F ; COC[C@@]12C[C@H](C)C[C@@H](C1)N2C(=O)Nc1cc(-c2ncccn2)c(C)cc1F]

The mixture of enantiomers was separated by SFC (ChiralPak IH, 250 mm×30 mm×10 μm; Mobile phase: A: CO₂, B: 0.1% NH₃H₂O in EtOH; B % in A: 50%-50%, 15 min; Flow rate: 70 g/min; Wavelength: 220 nm; Column temperature: 35° C.; System back pressure: 120 bar) to give trans-N-(2-fluoro-4-methyl-5-pyrimidin-2-ylphenyl)-1-(methoxymethyl)-3-methyl-6-azabicyclo[3.1.1]heptane-6-carboxamide (peak 1 in SFC) Example 199. LCMS: m/z=385.2 [M+H]⁺ and trans-N-(2-fluoro-4-methyl-5-pyrimidin-2-ylphenyl)-1-(methoxymethyl)-3-methyl-6-azabicyclo[3.1.1]heptane-6-carboxamide (peak 2 in SFC) Example 200. LCMS: m/z=385.2 [M+H]⁺.

### Examples 201 and 202

### (1S,8R)—N-(2-fluoro-4-methyl-5-pyridazin-3-ylphenyl)-9-azatricyclo[6.2.1.02,7]undeca-2(7),3,5-triene-9-carboxamide and (1R,8S)—N-(2-fluoro-4-methyl-5-pyridazin-3-ylphenyl)-9-azatricyclo[6.2.1.02,7]undeca-2(7),3,5-triene-9-carboxamide

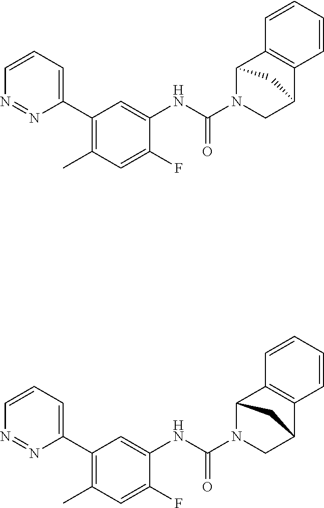 [SMILES: Cc1cc(F)c(NC(=O)N2C[C@H]3C[C@@H]2c2ccccc23)cc1-c1cccnn1 ; Cc1cc(F)c(NC(=O)N2C[C@@H]3C[C@H]2c2ccccc23)cc1-c1cccnn1]

The mixture of enantiomers was separated by SFC (DAICEL CHIRALPAK AD, 250 mm×30 mm, 10 m; Mobile phase: A: CO₂, B: 0.1% NH₃H₂O in EtOH; B % in A: 45%-45%, 15 min; Flow rate: 70 g/min; Wavelength: 220 nm; Column temperature: 35° C.; System back pressure: 120 bar) to give N-(2-fluoro-4-methyl-5-pyridazin-3-ylphenyl)-9-azatricyclo[6.2.1.02,7]undeca-2(7),3,5-triene-9-carboxamide (peak 1 in SFC) Example 240. LCMS: m/z=375.2 [M+H]⁺ and N-(2-fluoro-4-methyl-5-pyridazin-3-ylphenyl)-9-azatricyclo[6.2.1.02,7]undeca-2(7),3,5-triene-9-carboxamide (peak 2 in SFC) Example 241. LCMS: m/z=375.2 [M+H]⁺.

### Examples 203 and 204

### (1S,4S)—N-[3-(5-fluoropyrimidin-2-yl)-4-methylphenyl]-1-(2-methylpyrazol-3-yl)bicyclo[2.1.1]hexane-5-carboxamide and (1R,4R)—N-[3-(5-fluoropyrimidin-2-yl)-4-methylphenyl]-1-(2-methylpyrazol-3-yl)bicyclo[2.1.1]hexane-5-carboxamide

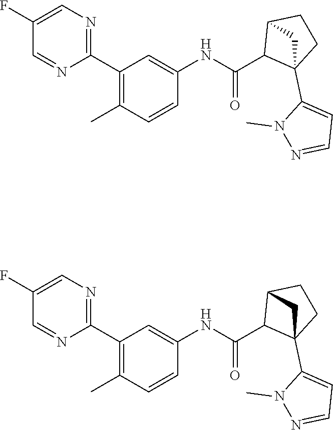 [SMILES: Cc1ccc(NC(=O)C2[C@H]3CC[C@]2(c2ccnn2C)C3)cc1-c1ncc(F)cn1 ; Cc1ccc(NC(=O)C2[C@@H]3CC[C@@]2(c2ccnn2C)C3)cc1-c1ncc(F)cn1]

The mixture of enantiomers was separated by SFC (ChiralPak IH, 250 mm×30 mm, 10 μm; mobile phase: A: CO₂, B: 0.1% IPA in EtOH; B % in A: 40%-40%, 16 min; Flow rate: 70 g/min, Wavelength: 220 nm; Column temperature: 35° C.; System back pressure: 140 bar) to give N-[3-(5-fluoropyrimidin-2-yl)-4-methylphenyl]-1-(2-methylpyrazol-3-yl)bicyclo[2.1.1]hexane-5-carboxamide (Peak 1 in SFC) Example 203. LCMS: m/z=392.1 [M+H]⁺ and N-[3-(5-fluoropyrimidin-2-yl)-4-methylphenyl]-1-(2-methylpyrazol-3-yl)bicyclo[2.1.1]hexane-5-carboxamide (Peak 2 in SFC) Example 204. LCMS: m/z=392.1 [M+H]⁺.

### Examples 205 and 206

### (2S,5S)-5-methyl-N-(4-methyl-3-pyrimidin-2-ylphenyl)-1-pyrimidin-2-ylpiperidine-2-carboxamide and (2R,5R)-5-methyl-N-(4-methyl-3-pyrimidin-2-ylphenyl)-1-pyrimidin-2-ylpiperidine-2-carboxamide

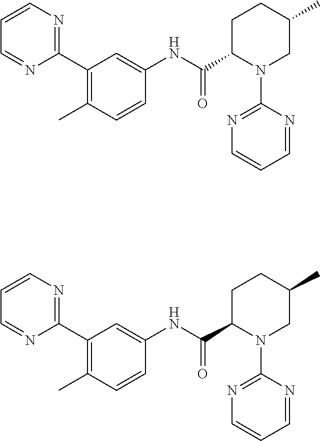 [SMILES: Cc1ccc(NC(=O)[C@@H]2CC[C@H](C)CN2c2ncccn2)cc1-c1ncccn1 ; Cc1ccc(NC(=O)[C@H]2CC[C@@H](C)CN2c2ncccn2)cc1-c1ncccn1]

The mixture of enantiomers was separated by SFC (DAICEL CHIRALPAK AD, 250 mm×30 mm, 10 m; Mobile phase: A: CO₂, B: 0.1% NH₃H₂O in IPA; B % in A: 5%-50%, 11 min; Flow rate: 70 g/min; Wavelength: 220 nm; Column temperature: 35° C.; System back pressure: 100 bar) to give cis-5-methyl-N-(4-methyl-3-(pyrimidin-2-yl)phenyl)-1-(pyrimidin-2-yl)piperidine-2-carboxamide (peak 1 in SFC) Example 205. LCMS: m/z=389.2 [M+H]⁺ and cis-5-methyl-N-(4-methyl-3-(pyrimidin-2-yl)phenyl)-1-(pyrimidin-2-yl)piperidine-2-carboxamide (peak 2 in SFC) Example 206. LCMS: m/z=389.2 [M+H]⁺.

### Examples 207 and 208

### (R)-6,6-dimethyl-N-(4-methyl-3-(pyrimidin-2-yl)phenyl)-4-(pyrimidin-2-yl)morpholine-3-carboxamide and (S)-6,6-dimethyl-N-(4-methyl-3-(pyrimidin-2-yl)phenyl)-4-(pyrimidin-2-yl)morpholine-3-carboxamide

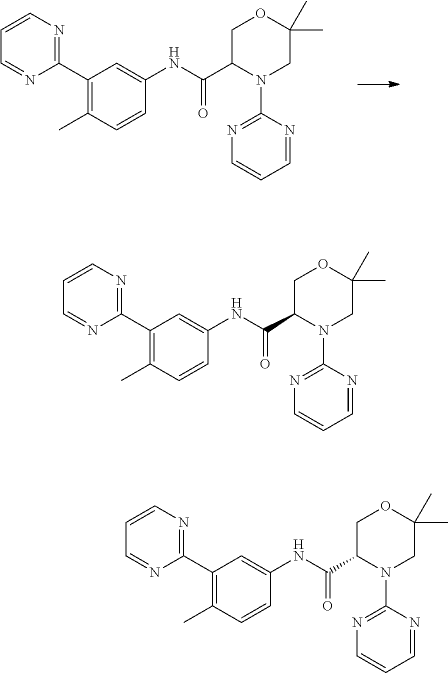 [SMILES: Cc1ccc(NC(=O)C2COC(C)(C)CN2c2ncccn2)cc1-c1ncccn1 ; Cc1ccc(NC(=O)[C@H]2COC(C)(C)CN2c2ncccn2)cc1-c1ncccn1 ; Cc1ccc(NC(=O)[C@@H]2COC(C)(C)CN2c2ncccn2)cc1-c1ncccn1]

The mixture of enantiomers was separated by SFC (DAICEL CHIRALPAK IH, 250 mm×30 mm, 10 in; Mobile phase: A: CO₂, B: 0.1% NH₃H₂O in EtOH; B3% in A: 35%, 11 min; Flow rate: 65 g/min; Wavelength: 220 nm; Column temperature: 40° C.; System back pressure: 100 bar) to give 6,6-dimethyl-N-(4-methyl-3-(pyrimidin-2-yl)phenyl)-4-(pyrimidin-2-yl)morpholine-3-carboxamide (peak 1 in SFC) Example 207. LCMS: m/z=405.2 [M+H]⁺ and 6,6-dimethyl-N-(4-methyl-3-(pyrimidin-2-yl)phenyl)-4-(pyrimidin-2-yl)morpholine-3-carboxamide (peak 2 in SFC) Example 208. LCMS: m/z=405.2 [M+H]⁺.

### Examples 209 and 210

### (1S,3R,5R)—N-(3-(1,2,4-triazin-3-yl)-4-(trifluoromethyl)phenyl)-1-(1-methoxyethyl)-3-methyl-6-azabicyclo[3.1.1]heptane-6-carboxamide and (1R,3S,5S)—N-(3-(1,2,4-triazin-3-yl)-4-(trifluoromethyl)phenyl)-1-(1-methoxyethyl)-3-methyl-6-azabicyclo[3.1.1]heptane-6-carboxamide

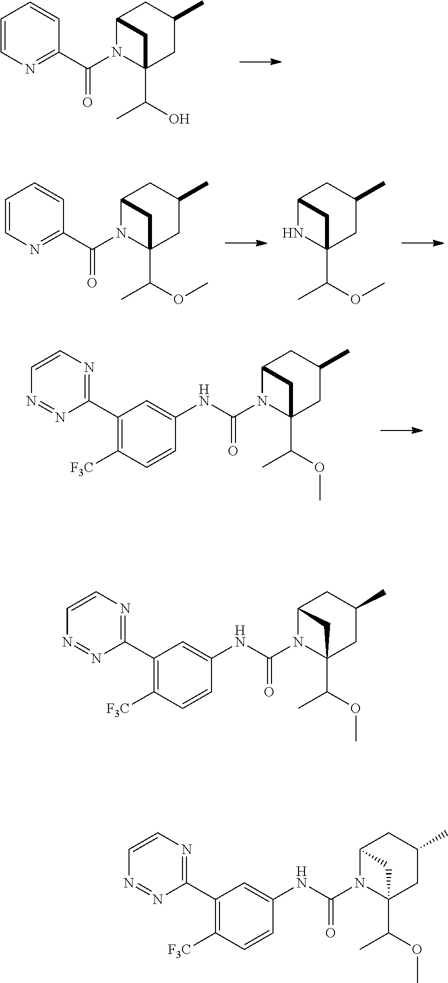

(cis-1-(1-methoxyethyl)-3-methyl-6-azabicyclo[3.1.1]heptan-6-yl)(pyridin-2-yl)methanone: To a solution of Intermediate 61, (cis-1-(1-hydroxyethyl)-3-methyl-6-azabicyclo[3.1.1]heptan-6-yl)(262yridine-2-yl)methanone (500 mg, 1.92 mmol), in DMF (2 mL) was added NaH (115 mg, 2.88 mmol, 60% purity) at 0° C. under N₂. The mixture was stirred at 0° C. for 0.5 h before MeI (409 mg, 2.88 mmol) was added to the mixture. The mixture was stirred at 20° C. for 12 h. The mixture was quenched by addition of sat. aq. NH₄Cl (30 mL) and extracted with EtOAc (3×10 mL). The combined organic layers were washed with brine (10 mL), dried over anhydrous Na₂SO₄, filtered and concentrated under reduced pressure. The resulting residue was purified by silica gel column chromatography (PE:EtOAc=3:1 to 1:1) to give the titled compound. LCMS: m/z=275.1 [M+H]⁺.

cis-1-(1-methoxyethyl)-3-methyl-6-azabicyclo[3.1.1]heptane: To a mixture of (cis-1-(1-methoxyethyl)-3-methyl-6-azabicyclo[3.1.1]heptan-6-yl)(pyridin-2-yl)methanone (200 mg, 0.73 mmol) in THF (5 mL) was added MeMgBr (1.21 mL, 3 M in Et₂O) at 0° C. under N₂. The mixture was stirred at 20° C. for 7 h. The reaction mixture was quenched by addition of sat. aq. NH₄Cl (1 mL). The mixture was concentrated under reduced pressure and the residue was slurried with DCM (20 mL), filtered and the filtrate was concentrated under reduce to give the titled compound. LCMS: m/z=170.3 [M+H]⁺.

cis-N-(3-(1,2,4-triazin-3-yl)-4-(trifluoromethyl)phenyl)-1-(1-methoxyethyl)-3-methyl-6-azabicyclo[3.1.1]heptane-6-carboxamide: To a mixture of triphosgene (62 mg, 0.21 mmol) in THF (5 mL) was added a mixture of TEA (126 mg, 1.25 mmol) and 3-(1,2,4-triazin-3-yl)-4-(trifluoromethyl) aniline (100 mg, 0.42 mmol) in THF (3 mL) at 0° C. under N₂. The mixture was stirred at 0° C. for 1 h. Then a solution of TEA (126 mg, 1.25 mmol) and cis-1-(1-methoxyethyl)-3-methyl-6-azabicyclo[3.1.1]heptane (106 mg, 0.62 mmol) in THF (3 mL) was added. The mixture was stirred at 20° C. for 2 h. The reaction mixture was diluted with H₂O (5 mL) and extracted with EtOAc (3×5 mL). The combined organic layers were washed with brine (5 mL), dried over anhydrous Na₂SO₄, filtered and concentrated under reduced pressure. The resulting residue was purified by prep-HPLC (column: Waters Xbridge BEH C18 100×30 mm, 5 m; mobile phase: A: 10 mM NH₄HCO₃ in water, B: MeCN; B in A: 40%-70% B, 8.0 min) to give the titled compound. LCMS: m/z=436.1 [M+H]⁺. [0520](1S,3R,5R)—N-(3-(1,2,4-triazin-3-yl)-4-(trifluoromethyl)phenyl)-1-(1-methoxyethyl)-3-methyl-6-azabicyclo[3.1.1]heptane-6-carboxamide and (1R,3S,5S)—N-(3-(1,2,4-triazin-3-yl)-4-(trifluoromethyl)phenyl)-1-(1-methoxyethyl)-3-methyl-6-azabicyclo[3.1.1]heptane-6-carboxamide: The mixture of enantiomers was separated by SFC separation (DAICEL CHIRALPAK AD, 250 mm×30 mm, 10 m; Mobile phase: A: CO₂, B: 0.1% NH₃H₂O in i-PrOH; B % in A: 45%-45%, 10 min; Flow rate: 70 g/min; Wavelength: 150 nm; Column temperature: 35° C.; System back pressure: 120 bar) to give cis-N-(3-(1,2,4-triazin-3-yl)-4-(trifluoromethyl)phenyl)-1-(1-methoxyethyl)-3-methyl-6-azabicyclo[3.1.1]heptane-6-carboxamide (peak 1 in SFC) Example 209. LCMS: m/z=436.1 [M+H]⁺ and cis-N-(3-(1,2,4-triazin-3-yl)-4-(trifluoromethyl)phenyl)-1-(1-methoxyethyl)-3-methyl-6-azabicyclo[3.1.1]heptane-6-carboxamide (peak 2 in SFC) Example 210. LCMS: m/z=436.1 [M+H]⁺.

### Examples 211 and 212

### (1S,3R,5R)—N-(3-(1,2,4-triazin-3-yl)-4-(trifluoromethyl)phenyl)-1-(1-methoxyethyl)-3-methyl-6-azabicyclo[3.1.1]heptane-6-carboxamide and (1R,3S,5S)—N-(3-(1,2,4-triazin-3-yl)-4-(trifluoromethyl)phenyl)-1-(1-methoxyethyl)-3-methyl-6-azabicyclo[3.1.1]heptane-6-carboxamide

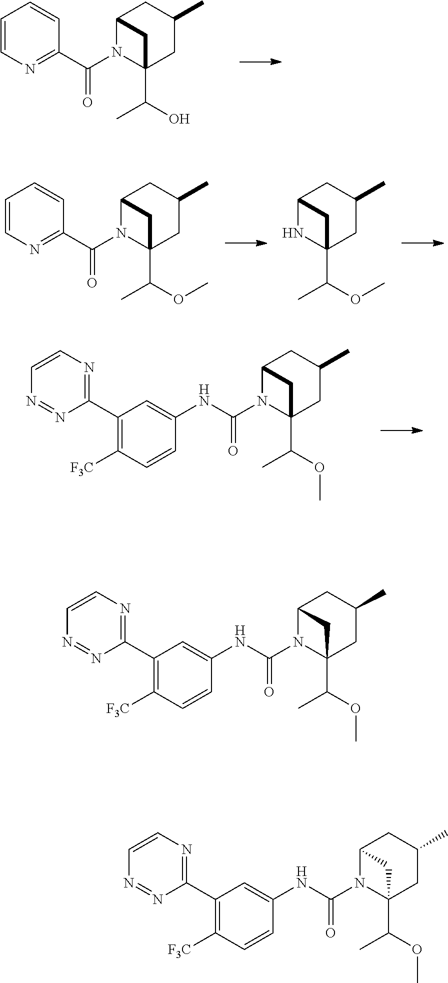

(cis-1-(1-methoxyethyl)-3-methyl-6-azabicyclo[3.1.1]heptan-6-yl)(pyridin-2-yl)methanone: To a solution of (Intermediate 62, cis-1-(1-hydroxyethyl)-3-methyl-6-azabicyclo[3.1.1]heptan-6-yl)(263yridine-2-yl)methanone (500 mg, 1.92 mmol), in DMF (6 mL) was added NaH (115 mg, 2.88 mmol, 60% in mineral oil) at 0° C. under N₂. The mixture was stirred at 0° C. for 0.5 h before MeI (409 mg, 2.88 mmol) was added to the reaction solution. The mixture was stirred at 20° C. for 12 h. The mixture was quenched by addition of sat. NH₄Cl (30 mL) and extracted with EtOAc (3×10 mL). The combined organic layers were washed with brine (10 mL), dried over anhydrous Na₂SO₄, filtered and concentrated under reduced pressure. The resulting residue was purified by silica gel column chromatography (PE:EtOAc=3:1 to 1:1) to give the titled compound. LCMS: m/z=275.1 [M+H]⁺.

cis-1-(1-methoxyethyl)-3-methyl-6-azabicyclo[3.1.1]heptane: To a mixture of (cis-1-(1-methoxyethyl)-3-methyl-6-azabicyclo[3.1.1]heptan-6-yl)(pyridin-2-yl)methanone (200 mg, 0.73 mmol) in THF (5 mL) was added MeMgBr (1.21 mL, 3 M in Et₂O) at 0° C. under N₂. The mixture was stirred at 20° C. for 14 h. The reaction mixture was quenched by addition of sat. NH₄Cl (1 mL) at 0° C. The mixture was concentrated under reduced pressure and the residue was slurried with DCM (20 mL), filtered and the filtrate was concentrated under reduce to give titled compound. LCMS: m/z=170.3 [M+H]⁺.

cis-N-(3-(1,2,4-triazin-3-yl)-4-(trifluoromethyl)phenyl)-1-(1-methoxyethyl)-3-methyl-6-azabicyclo[3.1.1]heptane-6-carboxamide: To a mixture of triphosgene (62 mg, 0.21 mmol) in THF (5 mL) was added a mixture of TEA (126 mg, 1.25 mmol) and 3-(1,2,4-triazin-3-yl)-4-(trifluoromethyl)aniline (100 mg, 0.44 mmol) in THF (3 mL) at 0° C. under N₂. The mixture was stirred at 0° C. for 1 h. Then a solution of TEA (126 mg, 1.25 mmol) and cis-1-(1-methoxyethyl)-3-methyl-6-azabicyclo[3.1.1]heptane (211 mg, 1.25 mmol) in THF (3 mL) was added at 0° C. The mixture was stirred at 20° C. for 2 h. The reaction mixture was diluted with H₂O (5 mL) and extracted with EtOAc (3×5 mL). The combined organic layers were washed with brine (5 mL), dried over anhydrous Na₂SO₄, filtered and concentrated under reduced pressure. The resulting residue was purified by prep-HPLC (column: Phenomenex Gemini-NX 80×40 mm×3 m; A: 10 mM NH₄HCO₃ in water, B: MeCN; B in A: 25%-55% B over 8.0 min) to give the titled compound. LCMS: m/z=436.3 [M+H]⁺.

(1S,3R,5R)—N-(3-(1,2,4-triazin-3-yl)-4-(trifluoromethyl)phenyl)-1-(1-methoxyethyl)-3-methyl-6-azabicyclo[3.1.1]heptane-6-carboxamide and (1R,3S,5S)—N-(3-(1,2,4-triazin-3-yl)-4-(trifluoromethyl)phenyl)-1-(1-methoxyethyl)-3-methyl-6-azabicyclo[3.1.1]heptane-6-carboxamide: The mixture of enantiomers was separated by SFC (DAICEL CHIRALPAK AD, 250 mm×30 mm, 10 m; Mobile phase: A: CO₂, B: 0.1% NH₃H₂O in i-PrOH; B % in A: 45%-45%, 10 min; Flow rate: 70 g/min; Wavelength: 150 nm; Column temperature: 35° C.; System back pressure: 120 bar) to give cis-N-(3-(1,2,4-triazin-3-yl)-4-(trifluoromethyl)phenyl)-1-(1-methoxyethyl)-3-methyl-6-azabicyclo[3.1.1]heptane-6-carboxamide (peak 1 in SFC) Example 211. LCMS: m/z=436.1 [M+H]⁺ and cis-N-(3-(1,2,4-triazin-3-yl)-4-(trifluoromethyl)phenyl)-1-(1-methoxyethyl)-3-methyl-6-azabicyclo[3.1.1]heptane-6-carboxamide (peak 2 in SFC) Example 212. LCMS: m/z=436.1 [M+H]⁺.

The following compounds were, or can be, made via similar procedures as those described above.

| Ex. | Structure | Name | NMR | LCMS |
|---|---|---|---|---|
| 213 | 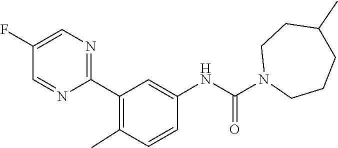 [SMILES: Cc1ccc(NC(=O)N2CCCC(C)CC2)cc1-c1ncc(F)cn1] | N-(3-(5- fluoropyrimidin-2-yl)-4- methylphenyl)-4- methylazepane-1- carboxamide | | m/z = 343.2 [M + H]⁺ |
| 214 | 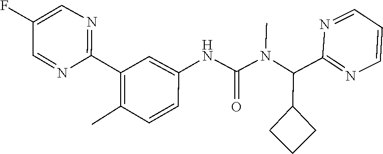 [SMILES: Cc1ccc(NC(=O)N(C)C(c2ncccn2)C2CCC2)cc1-c1ncc(F)cn1] | 1- (cyclobutyl(pyrimidin- 2-yl)methyl)-3-(3-(5- fluoropyrimidin-2-yl)-4- methylphenyl)-1- methylurea | | m/z = 407.1 [M + H]⁺ |
| 215 | 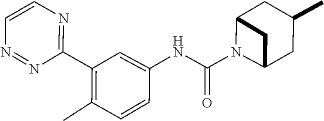 | cis-3-methyl-N-(4- methyl-3-(1,2,4-triazin- 3-yl)phenyl)-6- azabicyclo[3.1.1]heptane- 6-carboxamide | | m/z = 324.1 [M + H]⁺ |
| 216 | 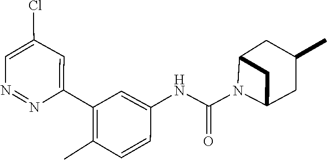 | cis-N-(3-(5- chloropyridazin-3-yl)-4- methylphenyl)-3- methyl-6- azabicyclo[3.1.1]heptane- 6-carboxamide | | m/z = 357.1 [M + H]⁺ |
| 217 | 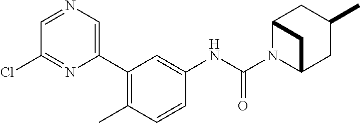 | cis-N-(3-(6- chloropyrazin-2-yl)-4- methylphenyl)-3- methyl-6- azabicyclo[3.1.1]heptane- 6-carboxamide | | m/z = 357.1 [M + H]⁺ |
| 218 | 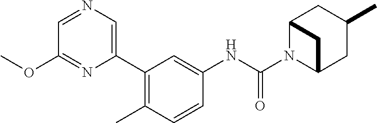 | cis-N-(3-(6- methoxypyrazin-2-yl)-4- methylphenyl)-3- methyl-6- azabicyclo[3.1.1]heptane- 6-carboxamide | | m/z = 353.2 [M + H]⁺ |
| 219 | 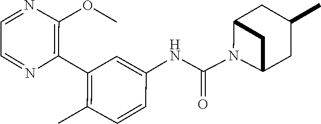 [SMILES: COc1nccnc1-c1cc(NC(=O)N2[C@@H]3C[C@@H](C)C[C@H]2C3)ccc1C] | cis-N-[3-(3- methoxypyrazin-2-yl)-4- methylphenyl]-3- methyl-6- azabicyclo[3.1.1]heptane- 6-carboxamide | | m/z = 353.2 [M + H]⁺ |
| 220 | 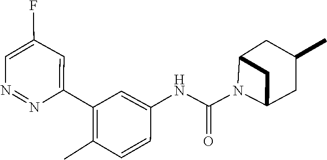 | cis-N-[3-(5- fluoropyridazin-3-yl)-4- methylphenyl]-3- methyl-6- azabicyclo[3.1.1]heptane- 6-carboxamide | | m/z = 341.1 [M + H]⁺ |
| 221 | 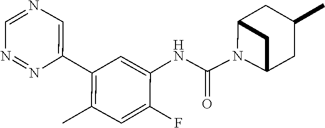 [SMILES: Cc1cc(F)c(NC(=O)N2[C@@H]3C[C@@H](C)C[C@H]2C3)cc1-c1cncnn1] | cis-N-[2-fluoro-4- methyl-5-(1,2,4-triazin- 6-yl)phenyl]-3-methyl- 6- azabicyclo[3.1.1]heptane- 6-carboxamide | | m/z = 342.1 [M + H]⁺ |
| 222 | 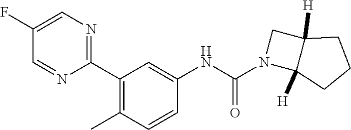 [SMILES: Cc1ccc(NC(=O)N2C[C@@H]3CCC[C@@H]32)cc1-c1ncc(F)cn1] | N-[3-(5- fluoropyrimidin-2-yl)-4- methylphenyl]-6- azabicyclo[3.2.0]heptane- 6-carboxamide | | m/z = 327.1 [M + H]⁺ |
| 223 | 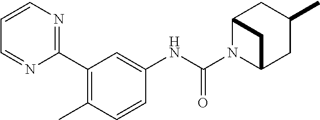 | cis-3-methyl-N-(4- methyl-3-pyrimidin-2- ylphenyl)-6- azabicyclo[3.1.1]heptane- 6-carboxamide | 1H NMR (400 MHz, CDCl3): δ 8.85 (d, J = 4.8 Hz, 2H), 7.70-7.65 (m, 2H), 7.26-7.21 (m, 2H), 6.17 (br s, 1H), 4.21 (t, J = 5.2 Hz, 2H), 2.68- | m/z = 323.2 [M + H]⁺ |
| | | | 2.54 (m, 3H), 2.51 | |
| | | | (s, 3H), 2.15-1.99 | |
| | | | (m, 1H), 1.37-1.31 | |
| | | | (m, 2H), 1.07 (d, | |
| | | | J = 8.4 Hz, 1H), | |
| | | | 1.03 (d, J = 6.8 | |
| | | | Hz, 3H) | |
| 224 | 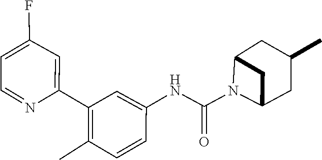 | cis-N-[3-(4- fluoropyridin-2-yl)-4- methylphenyl]-3- methyl-6- azabicyclo[3.1.1]heptane- 6-carboxamide | | m/z = 340.2 [M + H]⁺ |
| 225 | 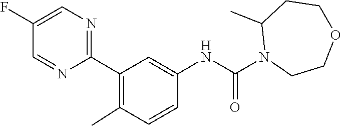 [SMILES: Cc1ccc(NC(=O)N2CCOCCC2C)cc1-c1ncc(F)cn1] | N-[3-(5- fluoropyrimidin-2-yl)-4- methylphenyl]-5- methyl-1,4-oxazepane- 4-carboxamide | | m/z = 345.1 [M + H]⁺ |
| 226 | 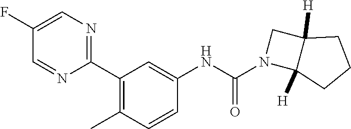 | N-[3-(5- fluoropyrimidin-2-yl)-4- methylphenyl]-7- azabicyclo[4.2.0]octane- 7-carboxamide | | m/z = 341.1 [M + H]⁺ |
| 227 | 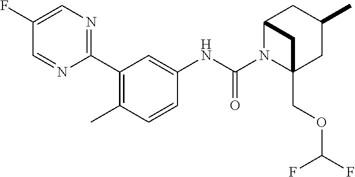 | cis-1- (difluoromethoxymethyl)- N-[3-(5- fluoropyrimidin-2-yl)-4- methylphenyl]-3- methyl-6- azabicyclo[3.1.1]heptane- 6-carboxamide | | m/z = 421.2 [M + H]⁺ |
| 228 | 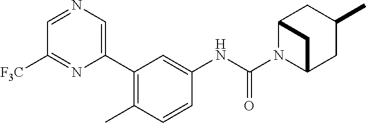 | cis-3-methyl-N-[4- methyl-3-[6- (trifluoromethyl)pyrazin- 2-yl]phenyl]-6- azabicyclo[3.1.1]heptane- 6-carboxamide | | m/z = 391.1 [M + H]⁺ |
| 229 | 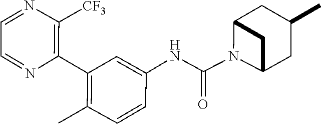 [SMILES: Cc1ccc(NC(=O)N2[C@@H]3C[C@@H](C)C[C@H]2C3)cc1-c1nccnc1C(F)(F)F] | cis-3-methyl-N-[4- methyl-3-[3- (trifluoromethyl)pyrazin- 2-yl]phenyl]-6- azabicyclo[3.1.1]heptane- 6-carboxamide | | m/z = 391.2 [M + H]⁺ |
| 230 | 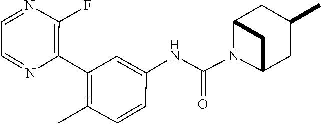 [SMILES: Cc1ccc(NC(=O)N2[C@@H]3C[C@@H](C)C[C@H]2C3)cc1-c1nccnc1F] | cis-N-[3-(3- fluoropyrazin-2-yl)-4- methylphenyl]-3- methyl-6- azabicyclo[3.1.1]heptane- 6-carboxamide | | m/z = 341.2 [M + H]⁺ |
| 231 | 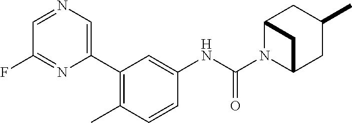 | cis-N-[3-(6- fluoropyrazin-2-yl)-4- methylphenyl]-3- methyl-6- azabicyclo[3.1.1]heptane- 6-carboxamide | | m/z = 341.1 [M + H]⁺ |
| 232 | 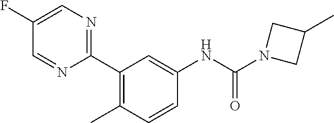 [SMILES: Cc1ccc(NC(=O)N2CC(C)C2)cc1-c1ncc(F)cn1] | N-[3-(5- fluoropyrimidin-2-yl)-4- methylphenyl]-3- methylazetidine-1- carboxamide | | m/z = 301.2 [M + H]⁺ |
| 233 | 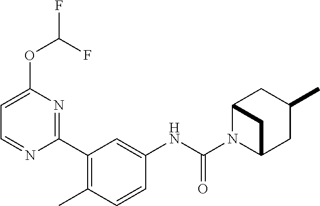 | cis-N-[3-[4- (difluoromethoxy) pyrimidin-2-yl]-4- methylphenyl]-3- methyl-6- azabicyclo[3.1.1]heptane- 6-carboxamide | | m/z = 389.2 [M + H]⁺ |
| 234 | 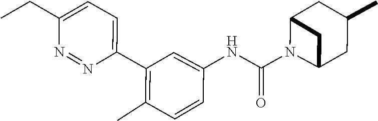 | cis-N-[3-(6- ethylpyridazin-3-yl)-4- methylphenyl]-3- methyl-6- azabicyclo[3.1.1]heptane- 6-carboxamide | | m/z = 351.2 [M + H]⁺ |
| 235 | 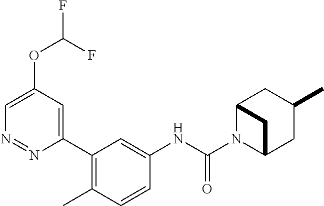 | cis-N-[3-[5- (difluoromethoxy) pyridazin-3-yl]-4- methylphenyl]-3- methyl-6- azabicyclo[3.1.1]heptane- 6-carboxamide | | m/z = 389.1 [M + H]⁺ |
| 236 | 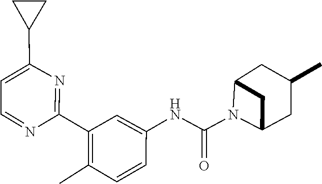 | cis-N-[3-(4- cyclopropylpyrimidin-2- yl)-4-methylphenyl]-3- methyl-6- azabicyclo[3.1.1]heptane- 6-carboxamide | | m/z = 363.2 [M + H]⁺ |
| 237 | 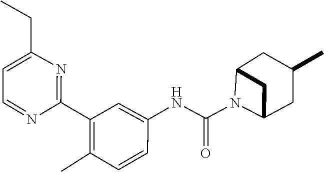 | cis-N-[3-(4- ethylpyrimidin-2-yl)-4- methylphenyl]-3- methyl-6- azabicyclo[3.1.1]heptane- 6-carboxamide | | m/z = 351.2 [M + H]⁺ |
| 238 | 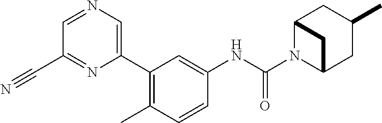 | cis-N-[3-(6- cyanopyrazin-2-yl)-4- methylphenyl]-3- methyl-6- azabicyclo[3.1.1]heptane- 6-carboxamide | | m/z = 348.2 [M + H]⁺ |
| 239 | 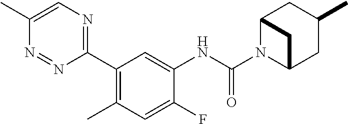 | cis-N-[2-fluoro-4- methyl-5-(6-methyl- 1,2,4-triazin-3- yl)phenyl]-3-methyl-6- azabicyclo[3.1.1]heptane- 6-carboxamide | | m/z = 356.2 [M + H]⁺ |
| 240 | 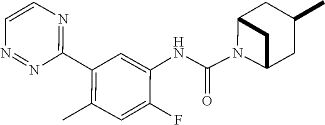 [SMILES: Cc1cc(F)c(NC(=O)N2[C@@H]3C[C@@H](C)C[C@H]2C3)cc1-c1nccnn1] | cis-N-[2-fluoro-4- methyl-5-(1,2,4-triazin- 3-yl)phenyl]-3-methyl- 6- azabicyclo[3.1.1]heptane- 6-carboxamide | | m/z = 342.1 [M + H]⁺ |
| 241 | 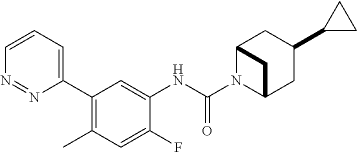 | cis-3-cyclopropyl-N-(2- fluoro-4-methyl-5- pyridazin-3-ylphenyl)-6- azabicyclo[3.1.1]heptane- 6-carboxamide | | m/z = 367.2 [M + H]⁺ |
| 242 | 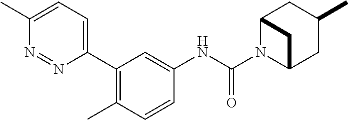 | cis-3-methyl-N-[4- methyl-3-(6- methylpyridazin-3- yl)phenyl]-6- azabicyclo[3.1.1]heptane- 6-carboxamide | | m/z = 337.1 [M + H]⁺ |
| 243 | 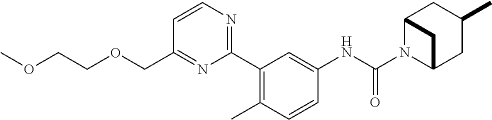 | cis-N-[3-[4-(2- methoxyethoxymethyl) pyrimidin-2-yl]-4- methylphenyl]-3- methyl-6- azabicyclo[3.1.1]heptane- 6-carboxamide | | m/z = 411.2 [M + H]⁺ |
| 244 | 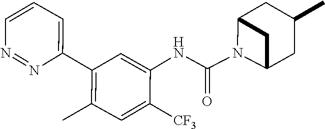 [SMILES: Cc1cc(C(F)(F)F)c(NC(=O)N2[C@@H]3C[C@@H](C)C[C@H]2C3)cc1-c1cccnn1] | cis-3-methyl-N-[4- methyl-5-pyridazin-3- yl-2- (trifluoromethyl)phenyl]- 6- azabicyclo[3.1.1]heptane- 6-carboxamide | | m/z = 391.2 [M + H]⁺ |
| 245 | 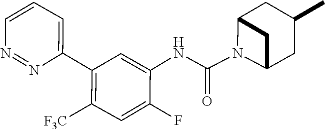 [SMILES: C[C@@H]1C[C@@H]2C[C@H](C1)N2C(=O)Nc1cc(-c2cccnn2)c(C(F)(F)F)cc1F] | cis-N-[2-fluoro-5- pyridazin-3-yl-4- (trifluoromethyl)phenyl]- 3-methyl-6- azabicyclo[3.1.1]heptane- 6-carboxamide | | m/z = 395.2 [M + H]⁺ |
| 246 | 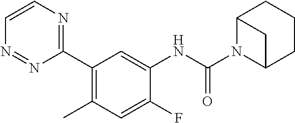 [SMILES: Cc1cc(F)c(NC(=O)N2C3CCCC2C3)cc1-c1nccnn1] | N-[2-fluoro-4-methyl-5- (1,2,4-triazin-3- yl)phenyl]-6- azabicyclo[3.1.1]heptane- 6-carboxamide | | m/z = 328.0 [M + H]⁺ |
| 247 | 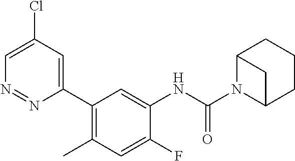 [SMILES: Cc1cc(F)c(NC(=O)N2C3CCCC2C3)cc1-c1cc(Cl)cnn1] | N-[5-(5- chloropyridazin-3-yl)-2- fluoro-4-methylphenyl]- 6- azabicyclo[3.1.1]heptane- 6-carboxamide | | m/z = 361.1 [M + H]⁺ |
| 248 | 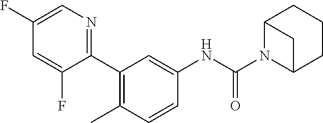 [SMILES: Cc1ccc(NC(=O)N2C3CCCC2C3)cc1-c1ncc(F)cc1F] | N-[3-(3,5- difluoropyridin-2-yl)-4- methylphenyl]-6- azabicyclo[3.1.1]heptane- 6-carboxamide | | m/z = 344.2 [M + H]⁺ |
| 249 | 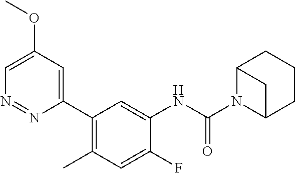 [SMILES: COc1cnnc(-c2cc(NC(=O)N3C4CCCC3C4)c(F)cc2C)c1] | N-[2-fluoro-5-(5- methoxypyridazin-3-yl)- 4-methylphenyl]-6- azabicyclo[3.1.1]heptane- 6-carboxamide | | m/z = 357.2 [M + H]⁺ |
| 250 | 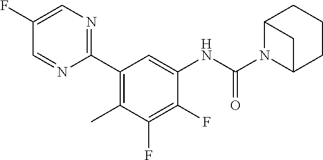 [SMILES: Cc1c(-c2ncc(F)cn2)cc(NC(=O)N2C3CCCC2C3)c(F)c1F] | N-[2,3-difluoro-5-(5- fluoropyrimidin-2-yl)-4- methylphenyl]-6- azabicyclo[3.1.1]heptane- 6-carboxamide | | m/z = 363.0 [M + H]⁺ |
| 251 | 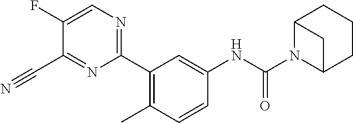 [SMILES: Cc1ccc(NC(=O)N2C3CCCC2C3)cc1-c1ncc(F)c(C#N)n1] | N-[3-(4-cyano-5- fluoropyrimidin-2-yl)-4- methylphenyl]-6- azabicyclo[3.1.1]heptane- 6-carboxamide | | m/z = 352.2 [M + H]⁺ |
| 252 | 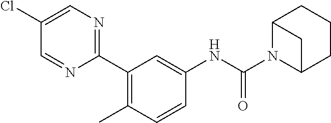 [SMILES: Cc1ccc(NC(=O)N2C3CCCC2C3)cc1-c1ncc(Cl)cn1] | N-[3-(5- chloropyrimidin-2-yl)-4- methylphenyl]-6- azabicyclo[3.1.1]heptane- 6-carboxamide | | m/z = 343.1 [M + H]⁺ |
| 253 | 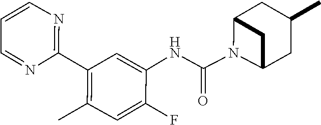 [SMILES: Cc1cc(F)c(NC(=O)N2[C@@H]3C[C@@H](C)C[C@H]2C3)cc1-c1ncccn1] | cis-N-(2-fluoro-4- methyl-5-pyrimidin-2- ylphenyl)-3-methyl-6- azabicyclo[3.1.1]heptane- 6-carboxamide | | m/z = 341.2 [M + H]⁺ |
| 254 | 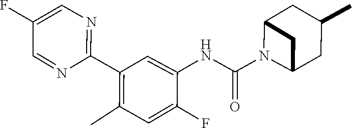 | cis-N-[2-fluoro-5-(5- fluoropyrimidin-2-yl)-4- methylphenyl]-3- methyl-6- azabicyclo[3.1.1]heptane- 6-carboxamide | | m/z = 359.2 [M + H]⁺ |
| 255 | 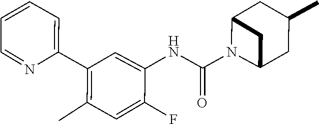 | cis-N-(2-fluoro-4- methyl-5-pyridin-2- ylphenyl)-3-methyl-6- azabicyclo[3.1.1]heptane- 6-carboxamide | | m/z = 340.2 [M + H]⁺ |
| 256 | 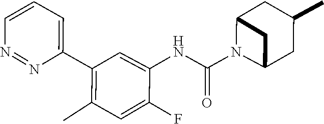 | cis-N-(2-fluoro-4- methyl-5-pyridazin-3- ylphenyl)-3-methyl-6- azabicyclo[3.1.1]heptane- 6-carboxamide | | m/z = 341.2 [M + H]⁺ |
| 257 | 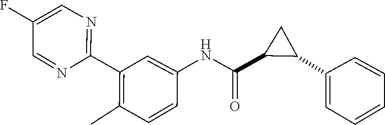 [SMILES: Cc1ccc(NC(=O)[C@H]2C[C@@H]2c2ccccc2)cc1-c1ncc(F)cn1] | trans-N-(3-(5- fluoropyrimidin-2-yl)-4- methylphenyl)-2- phenylcyclopropane-1- carboxamide | | m/z = 348.0 [M + H]⁺ |
| 258 | 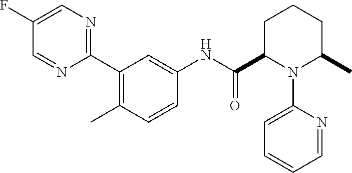 | cis-N-(3-(5- fluoropyrimidin-2-yl)-4- methylphenyl)-6- methyl-1-(pyridin-2- yl)piperidine-2- carboxamide | | m/z = 406.1 [M + H]⁺ |
| 259 | 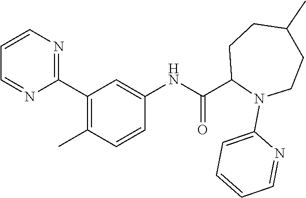 [SMILES: Cc1ccc(NC(=O)C2CCC(C)CCN2c2ccccn2)cc1-c1ncccn1] | 5-methyl-N-(4-methyl- 3-(pyrimidin-2- yl)phenyl)-1-(pyridin-2- yl)azepane-2- carboxamide | | m/z = 402.2 [M + H]⁺ |
| 260 | 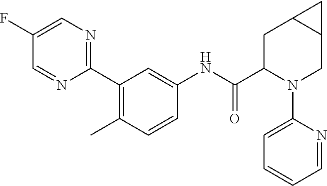 [SMILES: Cc1ccc(NC(=O)C2CC3CC3CN2c2ccccn2)cc1-c1ncc(F)cn1] | N-(3-(5- fluoropyrimidin-2-yl)-4- methylphenyl)-3- (pyridin-2-yl)-3- azabicyclo[4.1.0]heptane- 4-carboxamide | | m/z = 404.2 [M + H]⁺ |
| 261 | 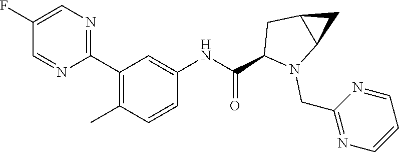 [SMILES: Cc1ccc(NC(=O)[C@H]2C[C@H]3C[C@H]3N2Cc2ncccn2)cc1-c1ncc(F)cn1] | (1R,3R,5R)-N-(3-(5- fluoropyrimidin-2-yl)-4- methylphenyl)-2-((5- methyl-1,3,4-oxadiazol- 2-yl)methyl)-2- azabicyclo[3.1.0]hexane- 3-carboxamide | | m/z = 405.2 [M + H]⁺ |
| 262 | 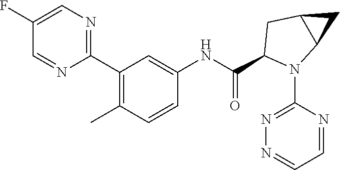 [SMILES: Cc1ccc(NC(=O)[C@H]2C[C@H]3C[C@H]3N2c2nccnn2)cc1-c1ncc(F)cn1] | (1R,3R,5R)-N-(3-(5- fluoropyrimidin-2-yl)-4- methylphenyl)-2-(1,2,4- triazin-3-yl)-2- azabicyclo[3.1.0]hexane- 3-carboxamide | | m/z = 392.2 [M + H]⁺ |
| 263 | 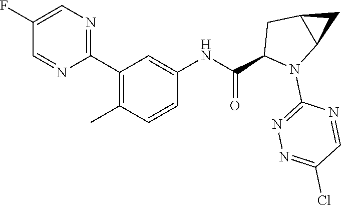 [SMILES: Cc1ccc(NC(=O)[C@H]2C[C@H]3C[C@H]3N2c2ncc(Cl)nn2)cc1-c1ncc(F)cn1] | (1R,3R,5R)-2-(6-chloro- 1,2,4-triazin-3-yl)-N-[3- (5-fluoropyrimidin-2- yl)-4-methylphenyl]-2- azabicyclo[3.1.0]hexane- 3-carboxamide | | m/z = 426.1 [M + H]⁺ |
| 264 | 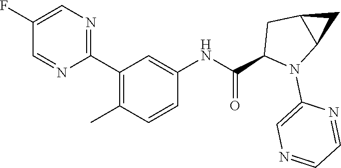 [SMILES: Cc1ccc(NC(=O)[C@H]2C[C@H]3C[C@H]3N2c2cnccn2)cc1-c1ncc(F)cn1] | (1R,3R,5R)-N-[3-(5- fluoropyrimidin-2-yl)-4- methylphenyl]-2- pyrazin-2-yl-2- azabicyclo[3.1.0]hexane- 3-carboxamide | ¹H NMR (400 MHz, CDCl₃): δ 8.79 (s, 1H), 8.68 (s, 2H), 8.31 (s, 1H), 8.03 (s, 1H), 7.89 (s, 1H), 7.82 (d, J = 2.0 Hz, 1H), 7.53 (dd, J = 2.4, 8.4 Hz, 1H), 7.21 (d, J = 8.0 Hz, 1H), 5.11-5.07 | m/z = 391.1 [M + H]⁺ |
| | | | (m, 1H), 3.65-3.60 | |
| | | | (m, 1H), 2.66-2.62 | |
| | | | (m, 1H), 2.54-2.51 | |
| | | | (m, 1H), 2.47 (s, | |
| | | | 3H), 1.72-1.69 (m, | |
| | | | 1H), 1.05-1.02 (m, | |
| | | | 1H), 1.01-0.95 (m, | |
| | | | 1H) | |
| 265 | 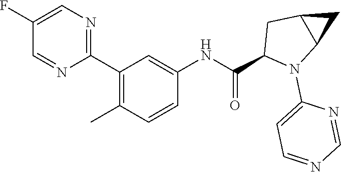 [SMILES: Cc1ccc(NC(=O)[C@H]2C[C@H]3C[C@H]3N2c2ccncn2)cc1-c1ncc(F)cn1] | (1R,3R,5R)-N-[3-(5- fluoropyrimidin-2-yl)-4- methylphenyl]-2- pyrimidin-4-yl-2- azabicyclo[3.1.0]hexane- 3-carboxamide | | m/z = 391.2 [M+H] + |
| 266 | 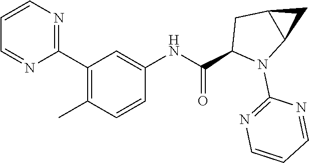 [SMILES: Cc1ccc(NC(=O)[C@H]2C[C@H]3C[C@H]3N2c2ncccn2)cc1-c1ncccn1] | (1R,3R,5R)-N-(4- methyl-3-pyrimidin-2- ylphenyl)-2-pyrimidin- 2-yl-2- azabicyclo[3.1.0]hexane- 3-carboxamide | | m/z = 373.2 [M + H]⁺ |
| 267 | 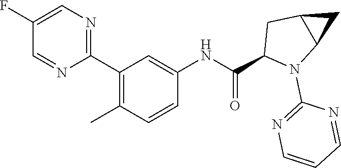 [SMILES: Cc1ccc(NC(=O)[C@H]2C[C@H]3C[C@H]3N2c2ncccn2)cc1-c1ncc(F)cn1] | (1R,3R,5R)-N-[3-(5- fluoropyrimidin-2-yl)-4- methylphenyl]-2- pyrimidin-2-yl-2- azabicyclo[3.1.0]hexane- 3-carboxamide | | m/z = 391.1 [M + H]⁺ |
| 268 | 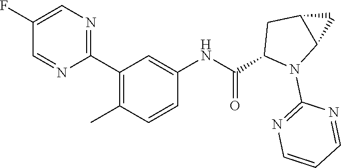 [SMILES: Cc1ccc(NC(=O)[C@@H]2C[C@@H]3C[C@@H]3N2c2ncccn2)cc1-c1ncc(F)cn1] | (1S,3S,5S)-N-[3-(5- fluoropyrimidin-2-yl)-4- methylphenyl]-2- pyrimidin-2-yl-2- azabicyclo[3.1.0]hexane- 3-carboxamide | | m/z = 391.2 [M + H]⁺ |
| 269 | 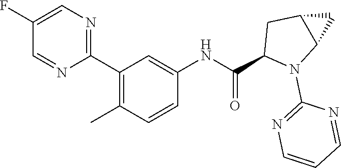 [SMILES: Cc1ccc(NC(=O)[C@H]2C[C@@H]3C[C@@H]3N2c2ncccn2)cc1-c1ncc(F)cn1] | (1S,3R,5S)-N-[3-(5- fluoropyrimidin-2-yl)-4- methylphenyl]-2- pyrimidin-2-yl-2- azabicyclo[3.1.0]hexane- 3-carboxamide | | m/z = 391.2 [M + H]⁺ |
| 270 | 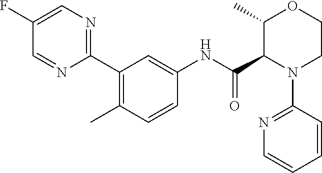 [SMILES: Cc1ccc(NC(=O)[C@H]2[C@H](C)OCCN2c2ccccn2)cc1-c1ncc(F)cn1] | (2S,3R)-N-(3-(5- fluoropyrimidin-2-yl)-4- methylphenyl)-2- methyl-4-(pyridin-2- yl)morpholine-3- carboxamide | | m/z = 408.2 [M + H]⁺ |
| 271 | 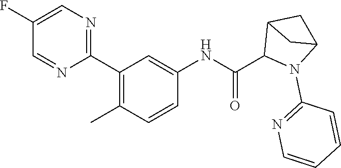 [SMILES: Cc1ccc(NC(=O)C2C3CC(C3)N2c2ccccn2)cc1-c1ncc(F)cn1] | N-(3-(5- fluoropyrimidin-2-yl)-4- methylphenyl)-2- (pyridin-2-yl)-2- azabicyclo[2.1.1]hexane- 3-carboxamide | | m/z = 390.2 [M + H]⁺ |
| 272 | 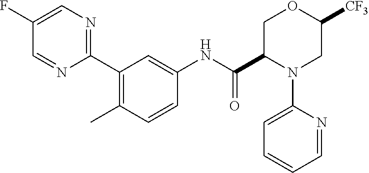 | cis-N-(3-(5- fluoropyrimidin-2-yl)-4- methylphenyl)-4- (pyridin-2-yl)-6- (trifluoromethyl) morpholine-3- carboxamide | | m/z = 462.1 [M + H]⁺ |
| 273 | 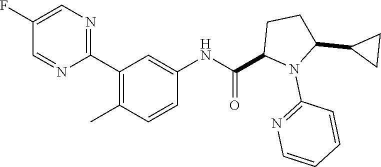 [SMILES: Cc1ccc(NC(=O)[C@H]2CC[C@@H](C3CC3)N2c2ccccn2)cc1-c1ncc(F)cn1] | cis-5-cyclopropyl-N-(3- (5-fluoropyrimidin-2- yl)-4-methylphenyl)-1- (pyridin-2- yl)pyrrolidine-2- carboxamide | | m/z = 418.2 [M + H]⁺ |
| 274 | 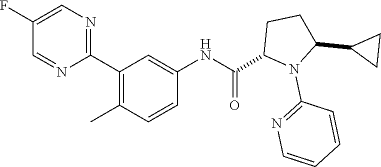 [SMILES: Cc1ccc(NC(=O)[C@@H]2CC[C@@H](C3CC3)N2c2ccccn2)cc1-c1ncc(F)cn1] | trans-5-cyclopropyl-N- (3-(5-fluoropyrimidin-2- yl)-4-methylphenyl)-1- (pyridin-2- yl)pyrrolidine-2- carboxamide | | m/z = 418.2 [M + H]⁺ |
| 275 | 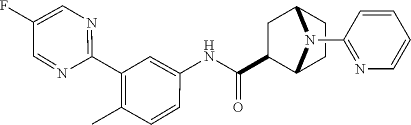 | cis-N-(3-(5- fluoropyrimidin-2-yl)-4- methylphenyl)-7- (pyridin-2-yl)-7- azabicyclo[2.2.1]heptane- 2-carboxamide | | m/z = 404.2 [M + H]⁺ |
| 276 | 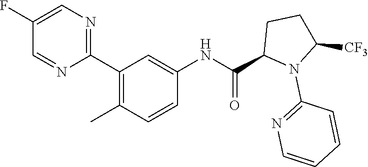 [SMILES: Cc1ccc(NC(=O)[C@H]2CC[C@@H](C(F)(F)F)N2c2ccccn2)cc1-c1ncc(F)cn1] | (2R,5S)-N-[3-(5- fluoropyrimidin-2-yl)-4- methylphenyl]-1- pyridin-2-yl-5- (trifluoromethyl) pyrrolidine-2-carboxamide | | m/z 446.2 [M + H]⁺ |
| 277 | 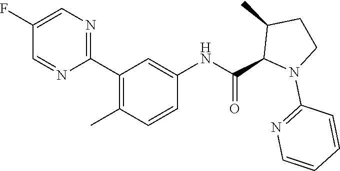 [SMILES: Cc1ccc(NC(=O)[C@H]2[C@@H](C)CCN2c2ccccn2)cc1-c1ncc(F)cn1] | (2R,3S)-N-[3-(5- fluoropyrimidin-2-yl)-4- methylphenyl]-3- methyl-1-pyridin-2- ylpyrrolidine-2- carboxamide | | m/z = 392.2 [M + H]⁺ |
| 278 | 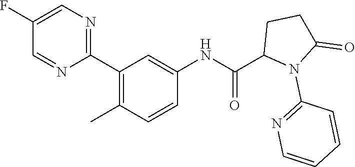 [SMILES: Cc1ccc(NC(=O)C2CCC(=O)N2c2ccccn2)cc1-c1ncc(F)cn1] | N-[3-(5- fluoropyrimidin-2-yl)-4- methylphenyl]-5-oxo-1- pyridin-2-ylpyrrolidine- 2-carboxamide | | m/z = 392.2 [M + H]⁺ |
| 279 | 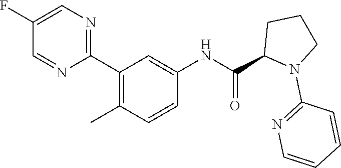 [SMILES: Cc1ccc(NC(=O)[C@H]2CCCN2c2ccccn2)cc1-c1ncc(F)cn1] | (2R)-N-[3-(5- fluoropyrimidin-2-yl)-4- methylphenyl]-1- pyridin-2-ylpyrrolidine- 2-carboxamide | | m/z = 378.2 [M + H]⁺ |
| 280 | 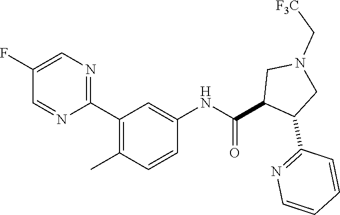 | trans-N-[3-(5- fluoropyrimidin-2-yl)-4- methylphenyl]-4- pyridin-2-yl-1-(2,2,2- trifluoroethyl)pyrrolidine- 3-carboxamide | | m/z = 460.2 [M + H]⁺ |
| 281 | 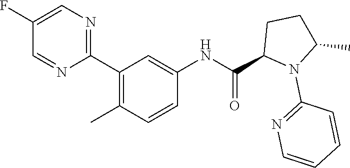 [SMILES: Cc1ccc(NC(=O)[C@H]2CC[C@H](C)N2c2ccccn2)cc1-c1ncc(F)cn1] | (2R,5S)-N-[3-(5- fluoropyrimidin-2-yl)-4- methylphenyl]-5- methyl-1-pyridin-2- ylpyrrolidine-2- carboxamide | | m/z = 392.2 [M + H]⁺ |
| 282 | 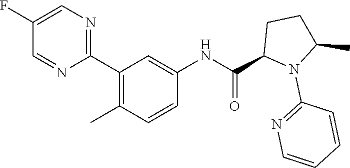 [SMILES: Cc1ccc(NC(=O)[C@H]2CC[C@@H](C)N2c2ccccn2)cc1-c1ncc(F)cn1] | (2R,5R)-N-[3-(5- fluoropyrimidin-2-yl)-4- methylphenyl]-5- methyl-1-pyridin-2- ylpyrrolidine-2- carboxamide | | m/z = 392.2 [M + H]⁺ |
| 283 | 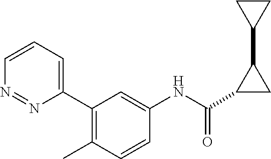 [SMILES: Cc1ccc(NC(=O)[C@H]2C[C@@H]2C2CC2)cc1-c1cccnn1] | trans-2-cyclopropyl-N- (4-methyl-3-pyridazin-3- ylphenyl)cyclopropane- 1-carboxamide | | m/z = 294.2 [M + H]⁺ |
| 284 | 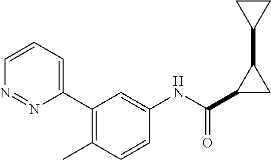 [SMILES: Cc1ccc(NC(=O)[C@@H]2C[C@@H]2C2CC2)cc1-c1cccnn1] | cis-2-cyclopropyl-N-(4- methyl-3-pyridazin-3- ylphenyl)cyclopropane- 1-carboxamide | | m/z = 294.1 [M + H]⁺ |
| 285 | 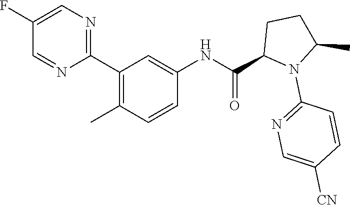 [SMILES: Cc1ccc(NC(=O)[C@H]2CC[C@@H](C)N2c2ccc(C#N)cn2)cc1-c1ncc(F)cn1] | (2R,5R)-1-(5- cyanopyridin-2-yl)-N- [3-(5-fluoropyrimidin-2- yl)-4-methylphenyl]-5- methylpyrrolidine-2- carboxamide | | m/z = 417.2 [M + H]⁺ |
| 286 | 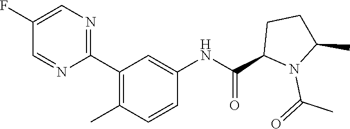 [SMILES: CC(=O)N1[C@H](C)CC[C@@H]1C(=O)Nc1ccc(C)c(-c2ncc(F)cn2)c1] | (2R,5R)-1-acetyl-N-[3- (5-fluoropyrimidin-2- yl)-4-methylphenyl]-5- methylpyrrolidine-2- carboxamide | | m/z = 357.2 [M + H]⁺ |
| 287 | 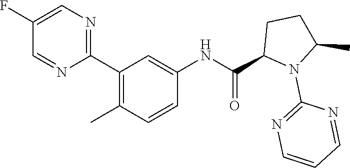 [SMILES: Cc1ccc(NC(=O)[C@H]2CC[C@@H](C)N2c2ncccn2)cc1-c1ncc(F)cn1] | (2R,5R)-N-[3-(5- fluoropyrimidin-2-yl)-4- methylphenyl]-5- methyl-1-pyrimidin-2- ylpyrrolidine-2- carboxamide | | m/z = 393.2 [M + H]⁺ |
| 288 | 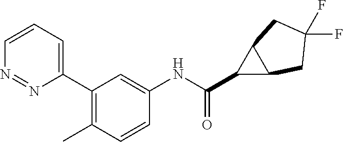 | cis-3,3-difluoro-N-(4- methyl-3-pyridazin-3- ylphenyl)bicyclo[3.1.0] hexane-6-carboxamide | | m/z = 330.2 [M + H]⁺ |
| 289 | 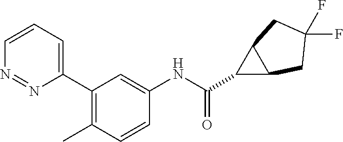 | trans-3,3-difluoro-N-(4- methyl-3-pyridazin-3- ylphenyl)bicyclo[3.1.0] hexane-6-carboxamide | | m/z = 330.0 [M + H]⁺ |
| 290 | 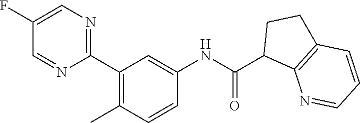 [SMILES: Cc1ccc(NC(=O)C2CCc3cccnc32)cc1-c1ncc(F)cn1] | N-[3-(5- fluoropyrimidin-2-yl)-4- methylphenyl]-6,7- dihydro-5H- cyclopenta[b]pyridine- 7-carboxamide | | m/z = 349.2 [M + H]⁺ |
| 291 | 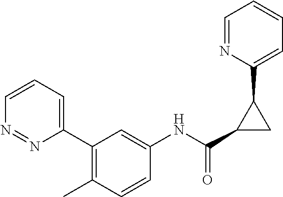 [SMILES: Cc1ccc(NC(=O)[C@@H]2C[C@@H]2c2ccccn2)cc1-c1cccnn1] | (1R,2S)-N-(4-methyl-3- pyridazin-3-ylphenyl)-2- pyridin-2- ylcyclopropane-1- carboxamide | | m/z = 331.2 [M + H]⁺ |
| 292 | 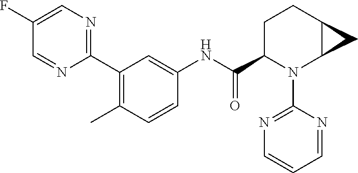 [SMILES: Cc1ccc(NC(=O)[C@H]2CC[C@H]3C[C@H]3N2c2ncccn2)cc1-c1ncc(F)cn1] | (1R,3R,6S)-N-[3-(5- fluoropyrimidin-2-yl)-4- methylphenyl]-2- pyrimidin-2-yl-2- azabicyclo[4.1.0]heptane- 3-carboxamide | | m/z = 405.1 [M + H]⁺ |
| 293 | 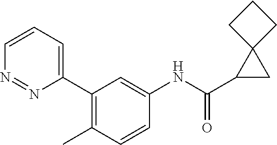 [SMILES: Cc1ccc(NC(=O)C2CC23CCC3)cc1-c1cccnn1] | N-(4-methyl-3- pyridazin-3- ylphenyl)spiro[2.3] hexane-2-carboxamide | | m/z = 294.2 [M + H]⁺ |
| 294 | 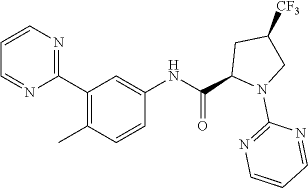 [SMILES: Cc1ccc(NC(=O)[C@H]2C[C@@H](C(F)(F)F)CN2c2ncccn2)cc1-c1ncccn1] | (2R,4R)-N-(4-methyl-3- pyridazin-3-ylphenyl)-1- pyrimidin-2-yl-4- (trifluoromethyl) pyrrolidine-2-carboxamide | | m/z = 429.2 [M + H]⁺ |
| 295 | 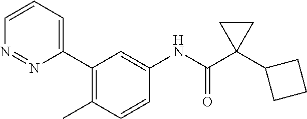 [SMILES: Cc1ccc(NC(=O)C2(C3CCC3)CC2)cc1-c1cccnn1] | 1-cyclobutyl-N-(4- methyl-3-pyridazin-3- ylphenyl)cyclopropane- 1-carboxamide | | m/z = 308.0 [M + H]⁺ |
| 296 | 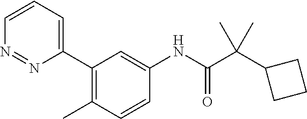 [SMILES: Cc1ccc(NC(=O)C(C)(C)C2CCC2)cc1-c1cccnn1] | 2-cyclobutyl-2-methyl- N-(4-methyl-3- pyridazin-3- ylphenyl)propanamide | | m/z = 310.0 [M + H]⁺ |
| 297 | 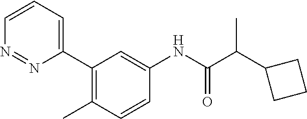 [SMILES: Cc1ccc(NC(=O)C(C)C2CCC2)cc1-c1cccnn1] | 2-cyclobutyl-N-(4- methyl-3-pyridazin-3- ylphenyl)propanamide | | m/z = 296.2 [M + H]⁺ |
| 298 | 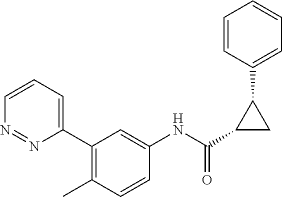 [SMILES: Cc1ccc(NC(=O)[C@H]2C[C@H]2c2ccccc2)cc1-c1cccnn1] | (1S,2R)-N-(4-methyl-3- pyridazin-3-ylphenyl)-2- phenylcyclopropane-1- carboxamide | | m/z = 330.2 [M + H]⁺ |
| 299 | 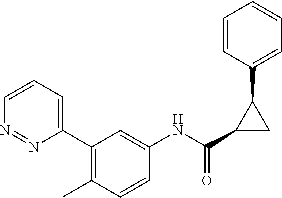 [SMILES: Cc1ccc(NC(=O)[C@@H]2C[C@@H]2c2ccccc2)cc1-c1cccnn1] | (1R,2S)-N-(4-methyl-3- pyridazin-3-ylphenyl)-2- phenylcyclopropane-1- carboxamide | | m/z = 330.2 [M + H]⁺ |
| 300 | 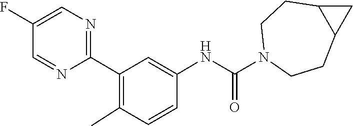 [SMILES: Cc1ccc(NC(=O)N2CCC3CC3CC2)cc1-c1ncc(F)cn1] | N-(3-(5- fluoropyrimidin-2-yl)-4- methylphenyl)-4- azabicyclo[5.1.0]octane- 4-carboxamide | | m/z = 341.1 [M + H]⁺ |
| 301 | 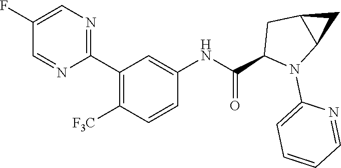 [SMILES: O=C(Nc1ccc(C(F)(F)F)c(-c2ncc(F)cn2)c1)[C@H]1C[C@H]2C[C@H]2N1c1ccccn1] | (1R,3R,5R)-N-(3-(5- fluoropyrimidin-2-yl)-4- (trifluoromethyl)phenyl)- 2-(pyridin-2-yl)-2- azabicyclo[3.1.0]hexane- 3-carboxamide | | m/z = 444.0 [M + H]⁺ |
| 302 | 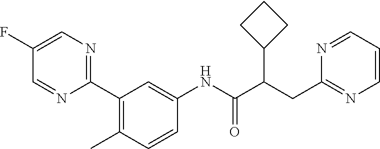 [SMILES: Cc1ccc(NC(=O)C(Cc2ncccn2)C2CCC2)cc1-c1ncc(F)cn1] | 2-cyclobutyl-N-(3-(5- fluoropyrimidin-2-yl)-4- methylphenyl)-3- (pyrimidin-2- yl)propanamide | | m/z = 392.2 [M + H]⁺ |
| 303 | 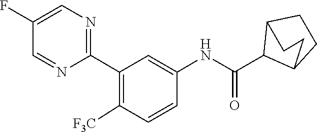 [SMILES: O=C(Nc1ccc(C(F)(F)F)c(-c2ncc(F)cn2)c1)C1C2CCC1CC2] | N-(3-(5- fluoropyrimidin-2-yl)-4- (trifluoromethyl)phenyl) bicyclo[2.2.1]heptane-e-7- carboxamide | | m/z = 380.0 [M + H]⁺ |
| 304 | 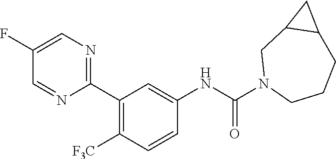 [SMILES: O=C(Nc1ccc(C(F)(F)F)c(-c2ncc(F)cn2)c1)N1CCCC2CC2C1] | N-(3-(5- fluoropyrimidin-2-yl)-4- (trifluoromethyl)phenyl)-3- azabicyclo[5.1.0]octane- 3-carboxamide | | m/z = 395.0 [M + H]⁺ |
| 305 | 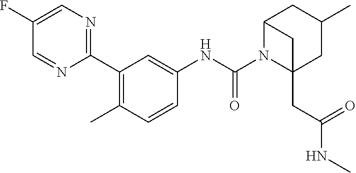 [SMILES: CNC(=O)CC12CC(C)CC(C1)N2C(=O)Nc1ccc(C)c(-c2ncc(F)cn2)c1] | N-(3-(5- fluoropyrimidin-2-yl)-4- methylphenyl)-3- methyl-1-(2- (methylamino)-2- oxoethyl)-6- azabicyclo[3.1.1]heptane- 6-carboxamide | | m/z = 412.1 [M + H]⁺ |
| 306 | 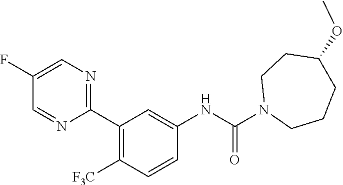 [SMILES: CO[C@@H]1CCCN(C(=O)Nc2ccc(C(F)(F)F)c(-c3ncc(F)cn3)c2)CC1] | (R)-N-(3-(5- fluoropyrimidin-2-yl)-4- (trifluoromethyl)phenyl)- 4-methoxyazepane-1- carboxamide | | m/z = 413.2 [M + H]⁺ |
| 307 | 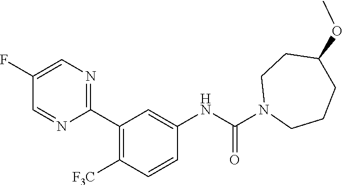 [SMILES: CO[C@H]1CCCN(C(=O)Nc2ccc(C(F)(F)F)c(-c3ncc(F)cn3)c2)CC1] | (S)-N-(3-(5- fluoropyrimidin-2-yl)-4- (trifluoromethyl)phenyl)- 4-methoxyazepane-1- carboxamide | | m/z = 413.2 [M + H]⁺ |
| 308 | 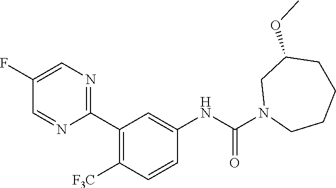 [SMILES: CO[C@@H]1CCCCN(C(=O)Nc2ccc(C(F)(F)F)c(-c3ncc(F)cn3)c2)C1] | (R)-N-(3-(5- fluoropyrimidin-2-yl)-4- (trifluoromethyl)phenyl)- 3-methoxyazepane-1- carboxamide | | m/z = 413.2 [M + H]⁺ |
| 309 | 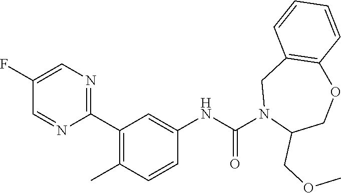 [SMILES: COCC1COc2ccccc2CN1C(=O)Nc1ccc(C)c(-c2ncc(F)cn2)c1] | N-(3-(5- fluoropyrimidin-2-yl)-4- methylphenyl)-3- (methoxymethyl)-2,3- dihydrobenzo[f][1,4] oxazepine-4(5H)- carboxamide | | m/z = 423.0 [M + H]⁺ |
| 310 | 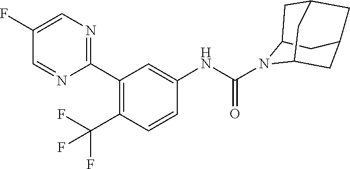 [SMILES: O=C(Nc1ccc(C(F)(F)F)c(-c2ncc(F)cn2)c1)N1C2CC3CC(C2)CC1C3] | N-(3-(5- fluoropyrimidin-2-yl)-4- (trifluoromethyl)phenyl)- 2-azaadamantane-2- carboxamide | | m/z = 421.1 [M + H]⁺ |
| 311 | 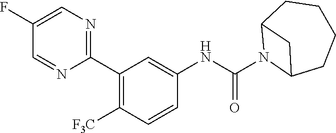 [SMILES: O=C(Nc1ccc(C(F)(F)F)c(-c2ncc(F)cn2)c1)N1C2CCCCC1C2] | N-(3-(5- fluoropyrimidin-2-yl)-4- (trifluoromethyl)phenyl)- 7- azabicyclo[4.1.1 ]octane- 7-carboxamide | | m/z = 395.1 [M + H]⁺ |
| 312 | 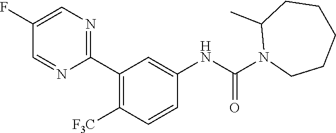 [SMILES: CC1CCCCCN1C(=O)Nc1ccc(C(F)(F)F)c(-c2ncc(F)cn2)c1] | N-(3-(5- fluoropyrimidin-2-yl)-4- (trifluoromethyl)phenyl)- 2-methylazepane-1- carboxamide | | m/z = 397.1 [M + H]⁺ |
| 313 | 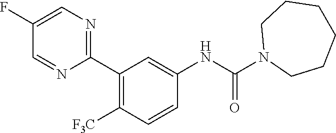 [SMILES: O=C(Nc1ccc(C(F)(F)F)c(-c2ncc(F)cn2)c1)N1CCCCCC1] | N-(3-(5- fluoropyrimidin-2-yl)-4- (trifluoromethyl)phenyl) azepane-1-carboxamide | | m/z = 383.1 [M + H]⁺ |
| 314 | 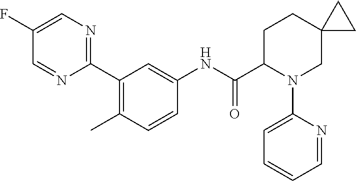 [SMILES: Cc1ccc(NC(=O)C2CCC3(CC3)CN2c2ccccn2)cc1-c1ncc(F)cn1] | | | m/z = 418.1 [M + H]⁺ |
| | mixture of enantiomers | | | |
| 315 | 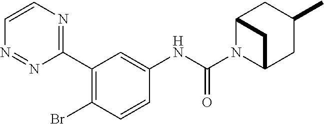 | | | m/z = 388.2, 390.2 [M + H]⁺ |
| 316 | 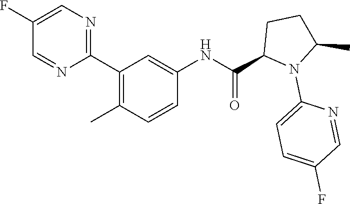 [SMILES: Cc1ccc(NC(=O)[C@H]2CC[C@@H](C)N2c2ccc(F)cn2)cc1-c1ncc(F)cn1] | | | m/z = 410.2 [M + H]⁺ |
| 317 | 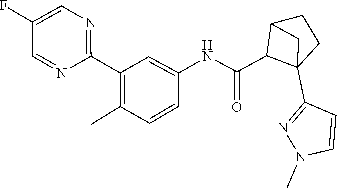 [SMILES: Cc1ccc(NC(=O)C2C3CCC2(c2ccn(C)n2)C3)cc1-c1ncc(F)cn1] | | | m/z = 392.2 [M + H]⁺ |
| | mixture of diastereomers | | | |
| 318 | 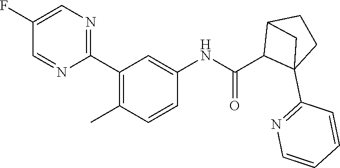 [SMILES: Cc1ccc(NC(=O)C2C3CCC2(c2ccccn2)C3)cc1-c1ncc(F)cn1] | | | m/z = 389.2 [M + H]⁺ |
| | mixture of diastereomers | | | |
| 319 | 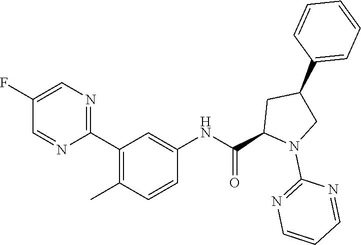 [SMILES: Cc1ccc(NC(=O)[C@H]2C[C@@H](c3ccccc3)CN2c2ncccn2)cc1-c1ncc(F)cn1] | | | m/z = 455.2 [M + H]⁺ |
| 320 | 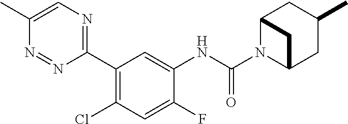 | | | m/z = 376.1 [M + H]⁺ |
| 321 | 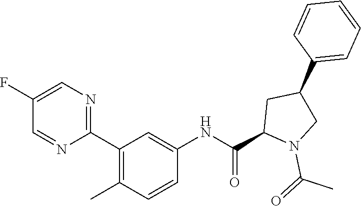 [SMILES: CC(=O)N1C[C@H](c2ccccc2)C[C@@H]1C(=O)Nc1ccc(C)c(-c2ncc(F)cn2)c1] | | | m/z = 419.2 [M + H]⁺ |
| 322 | 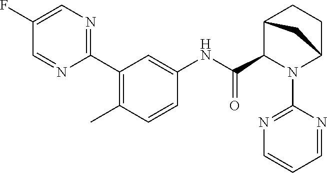 [SMILES: Cc1ccc(NC(=O)[C@H]2[C@@H]3CC[C@@H](C3)N2c2ncccn2)cc1-c1ncc(F)cn1] | | | m/z = 405.2 [M + H]⁺ |
| 323 | 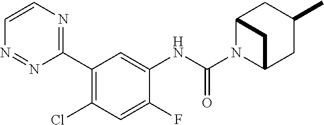 [SMILES: C[C@@H]1C[C@@H]2C[C@H](C1)N2C(=O)Nc1cc(-c2nccnn2)c(Cl)cc1F] | | | m/z = 362.3 [M + H]⁺ |
| 324 | 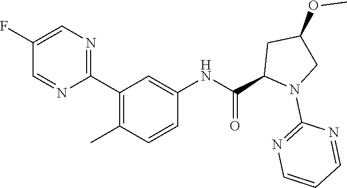 [SMILES: CO[C@@H]1C[C@H](C(=O)Nc2ccc(C)c(-c3ncc(F)cn3)c2)N(c2ncccn2)C1] | | | m/z = 409.2 [M + H]⁺ |
| 325 | 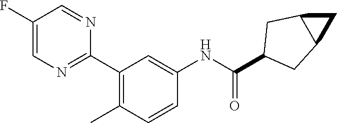 [SMILES: Cc1ccc(NC(=O)[C@@H]2C[C@@H]3C[C@@H]3C2)cc1-c1ncc(F)cn1] | | | m/z = 312.1 [M + H]⁺ |
| 326 | 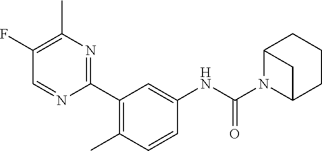 [SMILES: Cc1ccc(NC(=O)N2C3CCCC2C3)cc1-c1ncc(F)c(C)n1] | | | m/z = 341.1 [M + H]⁺ |
| 327 | 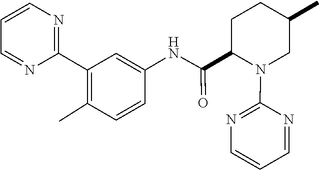 [SMILES: Cc1ccc(NC(=O)[C@H]2CC[C@@H](C)CN2c2ncccn2)cc1-c1ncccn1] | | | m/z = 389.2 [M + H]⁺ |
| | mixture of enantiomers | | | |
| 328 | 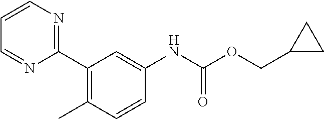 [SMILES: Cc1ccc(NC(=O)OCC2CC2)cc1-c1ncccn1] | | | m/z = 284.0 [M + H]⁺ |

### Biological Example 1

### Biochemical Assay of the Compounds

Preparation of full-length SARM1 (FL-SARM1) lysate: HEK293T cells (ATCC: CRL-3216) were grown on 150 mm TC-treated dishes to 80-90% confluency in complete DMEM (DMEM (Thermo Fisher: 11965175) supplemented with 10% HI—FBS (VWR: 10802-772), 1× Pen/Strep (Thermo Fisher: 15140122), 1×NEAA (Thermo Fisher: 1140050), 1× glutamax (Thermo Fisher: 35050061), and 1 mM sodium pyruvate (Thermo Fisher: 11360070)) at 37° C. and 5% CO₂. One hour prior to transfection, the media was replaced with fresh, 37° C. complete DMEM (20 mL per one 150 mm dish) supplemented with additional 10 mM glucose (Alfa Aesar AAJ60067EQE). Per one 150 mm dish, 30 μg FL-SARM1 (SEQ. ID. 1; cloned in-house) plasmid was dissolved in 1 mL DMEM at ambient temperature and were mixed by inverting the tube 8-10 times. 90 μL of GenJet™ in vitro DNA transfection reagent (Ver2) was dissolved in 1 mL DMEM at ambient temperature and were mixed by inverting the tube 8-10 times. The plasmid and transfection agent solutions were combined, mixed by 8-10 inversions and incubated for 10 minutes at ambient temperature. 2 mL of this transfection mixture was added to each dish containing HEK293T cells as prepared above followed by a gentle mixing of 4-5 horizontal rotations. The dishes were incubated at 37° C. and 5% CO₂ for 24 h. The dishes were removed from the incubator, the medium was aspirated and the cells were scraped off using cell scrapers in ice-cold 1×PBS (5 mL/dish, Thermo Fisher Scientific 10010023). The collected cells were centrifuged at 300 g for 5 minutes at 4° C. The supernatant was aspirated and the pellet was frozen at −80° C. until needed. The cell pellet from 30 dishes was dissolved in 30 mL 1×PBS supplemented with 4 tablets of Complete, Mini EDTA-free protease inhibitor cocktail at 4° C. This mixture was sonicated on ice for 10 minutes at 50% amplitude with a 1 second on/1 second off interval using a Model 120 sonicator (Thermo Fisher Scientific, FB120110). The lysate was centrifuged at 16000 g for 10 minutes at 4° C. Batches with supernatant possessing NMN-dependent SARM1 activity were selected, pooled, and stored at −80° C. until used in the FL-SARM1 cellular lysate assay described below.

To a white 384-well Proxiplate (PerkinElmer, PE-6008280) was added 50 nL/well of a DMSO solution containing test compounds followed by 7.5 μL/well of a 0.067 mg/mL solution of SARMi cellular lysate in reaction buffer (DPBS containing CHAPSO (0.1%) and fatty acid free BSA (0.032%)). The plate was centrifuged for 1 minute at 1000 RPM and then placed in an incubator at 23° C. for 15 minutes. To the wells were added 2.5 μL/well of a solution containing 40 M NAD⁺ and 4 mM nicotinamide mononucleotide (NMN) in reaction buffer. The plate was centrifuged for 1 min at 1000 RPM, the plate was sealed and placed in an incubator at 23° C. for 3.5 hours before adding 3.5 μL/well of NAD/NADH-Glo™ solution (preparation as described by Promega using the extended detection protocol). The plate was centrifuged for 1 minute at 1000 RPM and then incubated at 23° C. for 20 minutes. 1 μL/well of a 3.625 mM solution of menadione in DMSO was added and the plate was centrifuged for 1 minute at 1000 RPM. Relative light units (RLU) were recorded using an Envision plate reader at a height of 6.5 mm. Percent inhibition was calculated as follows:

$$\% inhibition = ( sample - low control ) / ( high control - low control ) \times 100.$$

IC₅₀ values were calculated from an 11 point curve using ½ log dilutions using a four-parameter logistic regression curve fit. Activity of the tested compounds is provided in Table 3 below as follows: ++=0.0001 μM<IC₅₀<1 μM; ++=IC₅₀ 1-10 μM; +=IC₅₀>10 μM.

TABLE 3

| | | Activity |
|---|---|---|
| Ex. | Activity | (μM) |
| 1 | + | 23 |
| 2 | +++ | 0.7 |
| 3 | ++ | 1.2 |
| 4 | +++ | 0.4 |
| 5 | +++ | 0.8 |
| 6 | ++ | 4.2 |
| 7 | ++ | 4.0 |
| 8 | +++ | 0.06 |
| 9 | +++ | 0.1 |
| 10 | +++ | 0.01 |
| 11 | +++ | 0.01 |
| 12 | ++ | 2.0 |
| 13 | +++ | 0.7 |
| 14 | +++ | 0.1 |
| 15 | +++ | 0.4 |
| 16 | ++ | 1.1 |
| 17 | +++ | 0.1 |
| 18 | +++ | 0.3 |
| 19 | +++ | 0.2 |
| 20 | +++ | 0.4 |
| 21 | ++ | 2.5 |
| 22 | ++ | 2.0 |
| 23 | +++ | 0.04 |
| 24 | ++ | 1.4 |
| 25 | ++ | 6.7 |
| 26 | ++ | 1.3 |
| 27 | +++ | 0.1 |
| 28 | +++ | 0.6 |
| 29 | +++ | 0.06 |
| 30 | +++ | 0.2 |
| 31 | +++ | 0.8 |
| 32 | ++ | 6.1 |
| 33 | ++ | 1.2 |
| 34 | ++ | 1.1 |
| 35 | ++ | 3.5 |
| 36 | ++ | 1.1 |
| 37 | +++ | 0.1 |
| 38 | ++ | 7.7 |
| 39 | ++ | 3.8 |
| 40 | +++ | 0.08 |
| 41 | +++ | 0.5 |
| 42 | +++ | 0.05 |
| 43 | +++ | 0.2 |
| 44 | +++ | 0.1 |
| 45 | +++ | 0.5 |
| 46 | +++ | 0.2 |
| 47 | +++ | 0.06 |
| 48 | +++ | 0.1 |
| 49 | +++ | 0.2 |
| 50 | +++ | 0.02 |
| 51 | +++ | 0.01 |
| 52 | +++ | 0.5 |
| 53 | +++ | 0.8 |
| 54 | +++ | 0.5 |
| 55 | +++ | 0.2 |
| 56 | ++ | 2.2 |
| 57 | ++ | 3.7 |
| 58 | + | 12 |
| 59 | +++ | 0.6 |
| 60 | ++ | 1.1 |
| 61 | + | 11 |
| 62 | +++ | 0.02 |
| 63 | +++ | 0.7 |
| 64 | +++ | 0.1 |
| 65 | ++ | 2.3 |
| 66 | +++ | 0.5 |
| 67 | +++ | 0.6 |
| 68 | ++ | 4.7 |
| 69 | +++ | 0.08 |
| 70 | ++ | 1.9 |
| 71 | + | 10.9 |
| 72 | ++ | 1.2 |
| 73 | +++ | 0.09 |
| 74 | +++ | 0.3 |
| 75 | +++ | 0.4 |
| 76 | ++ | 6.8 |
| 77 | +++ | 0.8 |
| 78 | +++ | 0.7 |
| 79 | ++ | 2.8 |
| 80 | + | 13 |
| 81 | ++ | 3.4 |
| 82 | ++ | 1.5 |
| 83 | + | 35 |
| 84 | ++ | 1.6 |
| 85 | + | 23 |
| 86 | +++ | 0.01 |
| 87 | + | 17 |
| 88 | + | 11 |
| 89 | + | 12 |
| 90 | + | 14 |
| 91 | + | 16 |
| 92 | ++ | 2.3 |
| 93 | + | 21 |
| 94 | + | 15 |
| 95 | ++ | 1.4 |
| 96 | +++ | 0.7 |
| 97 | + | 11 |
| 98 | + | 20 |
| 99 | + | 18 |
| 100 | +++ | 0.6 |
| 101 | ++ | 10 |
| 102 | +++ | 0.9 |
| 103 | +++ | 0.5 |
| 104 | ++ | 2.6 |
| 105 | + | 17 |
| 106 | ++ | 1.0 |
| 107 | + | 18 |
| 108 | ++ | 1.1 |
| 109 | +++ | 0.3 |
| 110 | ++ | 6.0 |
| 111 | ++ | 2.8 |
| 112 | ++ | 1.7 |
| 113 | ++ | 2.5 |
| 114 | +++ | 0.7 |
| 115 | + | 36 |
| 116 | ++ | 7.4 |
| 117 | ++ | 4.6 |
| 118 | ++ | 6.1 |
| 119 | ++ | 1.9 |
| 120 | + | 15 |
| 121 | +++ | 0.9 |
| 122 | +++ | 0.3 |
| 123 | +++ | 0.3 |
| 124 | ++ | 3.1 |
| 125 | ++ | 3.5 |
| 126 | ++ | 2.5 |
| 127 | ++ | 5.6 |
| 128 | ++ | 4.3 |
| 129 | ++ | 9.1 |
| 130 | ++ | 2.6 |
| 131 | + | 18 |
| 132 | + | 43 |
| 133 | + | 24 |
| 134 | ++ | 1.9 |
| 135 | ++ | 3.3 |
| 136 | ++ | 6.4 |
| 137 | +++ | 0.1 |
| 138 | +++ | 0.7 |
| 139 | +++ | 0.2 |
| 140 | +++ | 0.9 |
| 141 | + | 19 |
| 142 | ++ | 2.6 |
| 143 | ++ | 1.8 |
| 144 | ++ | 9.7 |
| 145 | ++ | 5.0 |
| 146 | ++ | 7.3 |
| 147 | +++ | 0.2 |
| 148 | + | 38 |
| 149 | + | 17 |
| 150 | ++ | 3.7 |
| 151 | ++ | 4.2 |
| 152 | ++ | 3.6 |
| 153 | + | 18 |
| 154 | ++ | 9.9 |
| 155 | ++ | 3.5 |
| 156 | ++ | 2.5 |
| 157 | + | 38 |
| 158 | + | 37 |
| 159 | ++ | 9.8 |
| 160 | ++ | 2.5 |
| 161 | ++ | 3.5 |
| 162 | ++ | 2.5 |
| 163 | + | 26 |
| 164 | ++ | 1.4 |
| 165 | ++ | 5.3 |
| 166 | + | 19 |
| 167 | + | 11 |
| 168 | ++ | 1.3 |
| 169 | + | 21 |
| 170 | ++ | 2.7 |
| 171 | + | 18 |
| 172 | ++ | 6.2 |
| 173 | ++ | 4.2 |
| 174 | + | 25 |
| 175 | ++ | 2.4 |
| 176 | + | 16 |
| 177 | + | 23 |
| 178 | ++ | 4.3 |
| 179 | +++ | 0.03 |
| 180 | +++ | 0.9 |
| 181 | ++ | 7.0 |
| 182 | ++ | 6.0 |
| 183 | +++ | 0.06 |
| 184 | +++ | 0.07 |
| 185 | +++ | 0.05 |
| 186 | +++ | 0.2 |
| 187 | +++ | 0.2 |
| 188 | +++ | 0.003 |
| 189 | ++ | 1.9 |
| 190 | +++ | 0.2 |
| 191 | +++ | 0.0009 |
| 192 | ++ | 1.0 |
| 193 | +++ | 0.1 |
| 194 | +++ | 0.01 |
| 195 | +++ | 0.3 |
| 196 | +++ | 0.8 |
| 197 | +++ | 0.4 |
| 198 | +++ | 0.3 |
| 199 | ++ | 4.3 |
| 200 | +++ | 0.1 |
| 201 | ++ | 2.0 |
| 202 | ++ | 6.7 |
| 203 | + | >50 |
| 204 | +++ | 0.5 |
| 205 | +++ | 0.3 |
| 206 | +++ | 0.7 |
| 207 | + | >50 |
| 208 | ++ | 3.3 |
| 209 | +++ | 0.5 |
| 210 | + | >50 |
| 211 | +++ | 0.004 |
| 212 | ++ | 9.6 |
| 213 | +++ | 0.2 |
| 214 | ++ | 3.9 |
| 215 | +++ | 0.03 |
| 216 | +++ | 0.01 |
| 217 | +++ | 0.04 |
| 218 | +++ | 0.03 |
| 219 | ++ | 1.0 |
| 220 | +++ | 0.1 |
| 221 | +++ | 0.6 |
| 222 | ++ | 3.4 |
| 223 | +++ | 0.04 |
| 224 | +++ | 0.04 |
| 225 | + | 32 |
| 226 | ++ | 1.5 |
| 227 | +++ | 0.02 |
| 228 | +++ | 0.09 |
| 229 | +++ | 0.2 |
| 230 | +++ | 0.04 |
| 231 | +++ | 0.08 |
| 232 | + | 20 |
| 233 | +++ | 0.1 |
| 234 | +++ | 0.4 |
| 235 | +++ | 0.06 |
| 236 | +++ | 0.7 |
| 237 | ++ | 1.1 |
| 238 | +++ | 0.04 |
| 239 | +++ | 0.2 |
| 240 | +++ | 0.09 |
| 241 | +++ | 0.09 |
| 242 | +++ | 0.07 |
| 243 | ++ | 1.1 |
| 244 | + | 16 |
| 245 | +++ | 0.2 |
| 246 | ++ | 2.0 |
| 247 | +++ | 0.4 |
| 248 | +++ | 0.2 |
| 249 | +++ | 0.9 |
| 250 | ++ | 1.5 |
| 251 | ++ | 1.0 |
| 252 | +++ | 0.5 |
| 253 | +++ | 0.08 |
| 254 | +++ | 0.02 |
| 255 | +++ | 0.02 |
| 256 | +++ | 0.1 |
| 257 | + | 30 |
| 258 | ++ | 2.4 |
| 259 | ++ | 5.8 |
| 260 | ++ | 1.3 |
| 261 | + | 34 |
| 262 | ++ | 2.5 |
| 263 | ++ | 1.8 |
| 264 | ++ | 1.1 |
| 265 | ++ | 1.9 |
| 266 | ++ | 4.2 |
| 267 | ++ | 3.1 |
| 268 | + | 33 |
| 269 | + | 47 |
| 270 | ++ | 9.5 |
| 271 | + | 17 |
| 272 | ++ | 1.1 |
| 273 | ++ | 1.9 |
| 274 | ++ | 1.2 |
| 275 | ++ | 4.9 |
| 276 | ++ | 6.3 |
| 277 | ++ | 1.4 |
| 278 | + | 17 |
| 279 | ++ | 4.2 |
| 280 | + | 28 |
| 281 | ++ | 1.3 |
| 282 | ++ | 1.2 |
| 283 | + | 11 |
| 284 | ++ | 1.8 |
| 285 | ++ | 2.2 |
| 286 | ++ | 1.7 |
| 287 | ++ | 2.1 |
| 288 | + | 15 |
| 289 | + | 26 |
| 290 | + | 17 |
| 291 | + | 16 |
| 292 | ++ | 2.0 |
| 293 | ++ | 2.4 |
| 294 | ++ | 9.4 |
| 295 | + | 13 |
| 296 | ++ | 4.3 |
| 297 | ++ | 6.5 |
| 298 | +++ | 0.6 |
| 299 | + | 14 |
| 300 | ++ | 1.2 |
| 301 | ++ | 2.1 |
| 302 | ++ | 3.0 |
| 303 | +++ | 0.5 |
| 304 | ++ | 2.4 |
| 305 | +++ | 0.01 |
| 306 | ++ | 9.6 |
| 307 | + | 13 |
| 308 | + | 32 |
| 309 | ++ | 1.1 |
| 310 | +++ | 0.7 |
| 311 | +++ | 0.1 |
| 312 | ++ | 1.4 |
| 313 | +++ | 0.8 |
| 314 | ++ | 1.2 |
| 315 | +++ | 0.08 |
| 316 | ++ | 1.7 |
| 317 | ++ | 1.0 |
| 318 | ++ | 1.5 |
| 319 | ++ | 6.7 |
| 320 | +++ | 0.6 |
| 321 | + | 24 |
| 322 | + | 13 |
| 323 | +++ | 0.2 |
| 324 | + | 17 |
| 325 | ++ | 1.2 |
| 326 | ++ | 1.4 |
| 327 | +++ | 0.5 |
| 328 | + | 31 |

FL-SARM1 plasmid sequence (SEQ. ID. 1):
tggaagggctaattcactcccaaagaagacaagatatccttgatctgtggatctaccacacacaaggcta
cttccctgattagcagaactacacaccagggccaggggtcagatatccactgacctttggatggtgctac
aagctagtaccagttgagccagataaggtagaagaggccaataaaggagagaacaccagcttgttacacc
ctgtgagcctgcatgggatggatgacccggagagagaagtgttagagtggaggtttgacagccgcctagc
atttcatcacgtggcccgagagctgcatccggagtacttcaagaactgctgatatcgagcttgctacaag
ggactttccgctggggactttccagggaggcgtggcctgggcgggactggggagtggcgagccctcagat
cctgcatataagcagctgctttttgcctgtactgggtctctctggttagaccagatctgagcctgggagc
tctctggctaactagggaacccactgcttaagcctcaataaagcttgccttgagtgcttcaagtagtgtg
tgcccgtctgttgtgtgactctggtaactagagatccctcagacccttttagtcagtgtggaaaatctct
agcagtggcgcccgaacagggacttgaaagcgaaagggaaaccagaggagctctctcgacgcaggactcg
gcttgctgaagcgcgcacggcaagaggcgaggggcggcgactggtgagtacgccaaaaattttgactagc
ggaggctagaaggagagagatgggtgcgagagcgtcagtattaagcgggggagaattagatcgcgatggg
aaaaaattcggttaaggccagggggaaagaaaaaatataaattaaaacatatagtatgggcaagcaggga
gctagaacgattcgcagttaatcctggcctgttagaaacatcagaaggctgtagacaaatactgggacag
ctacaaccatcccttcagacaggatcagaagaacttagatcattatataatacagtagcaaccctctatt
gtgtgcatcaaaggatagagataaaagacaccaaggaagctttagacaagatagaggaagagcaaaacaa
aagtaagaccaccgcacagcaagcggccggccgctgatcttcagacctggaggaggagatatgagggaca
attggagaagtgaattatataaatataaagtagtaaaaattgaaccattaggagtagcacccaccaaggc
aaagagaagagtggtgcagagagaaaaaagagcagtgggaataggagctttgttccttgggttcttggga
gcagcaggaagcactatgggcgcagcgtcaatgacgctgacggtacaggccagacaattattgtctggta
tagtgcagcagcagaacaatttgctgagggctattgaggcgcaacagcatctgttgcaactcacagtctg
gggcatcaagcagctccaggcaagaatcctggctgtggaaagatacctaaaggatcaacagctcctgggg
atttggggttgctctggaaaactcatttgcaccactgctgtgccttggaatgctagttggagtaataaat
ctctggaacagatttggaatcacacgacctggatggagtgggacagagaaattaacaattacacaagctt
aatacactccttaattgaagaatcgcaaaaccagcaagaaaagaatgaacaagaattattggaattagat
aaatgggcaagtttgtggaattggtttaacataacaaattggctgtggtatataaaattattcataatga
tagtaggaggcttggtaggtttaagaatagtttttgctgtactttctatagtgaatagagttaggcaggg
atattcaccattatcgtttcagacccacctcccaaccccgaggggacccgacaggcccgaaggaatagaa
gaagaaggtggagagagagacagagacagatccattcgattagtgaacggatctcgacggtatcgccttt
aaaagaaaaggggggattggggggtacagtgcaggggaaagaatagtagacataatagcaacagacatac
aaactaaagaattacaaaaacaaattacaaaaattcaaaattttcgggtttattacagggacagcagaga
tccagtttatcgatgagtaattcatacaaaaggactcgcccctgccttggggaatcccagggaccgtcgt
taaactcccactaacgtagaacccagagatcgctgcgttcccgccccctcacccgcccgctctcgtcatc
actgaggtggagaagagcatgcgtgaggctccggtgcccgtcagtgggcagagcgcacatcgcccacagt
ccccgagaagttggggggaggggtcggcaattgaaccggtgcctagagaaggtggcgcggggtaaactgg
gaaagtgatgtcgtgtactggctccgcctttttcccgagggtgggggagaaccgtatataagtgcagtag
tcgccgtgaacgttctttttcgcaacgggtttgccgccagaacacaggtaagtgccgtgtgtggttcccg
cgggcctggcctctttacgggttatggcccttgcgtgccttgaattacttccacgcccctggctgcagta
cgtgattcttgatcccgagcttcgggttggaagtgggtgggagagttcgaggccttgcgcttaaggagcc
ccttcgcctcgtgcttgagttgaggcctggcttgggcgctggggccgccgcgtgcgaatctggtggcacc
ttcgcgcctgtctcgctgctttcgataagtctctagccatttaaaatttttgatgacctgctgcgacgct
ttttttctggcaagatagtcttgtaaatgcgggccaagatctgcacactggtatttcggtttttggggcc
gcgggcggcgacggggcccgtgcgtcccagcgcacatgttcggcgaggcggggcctgcgagcgcggccac
cgagaatcggacgggggtagtctcaagctggccggcctgctctggtgcctggcctcgcgccgccgtgtat
cgccccgccctgggcggcaaggctggcccggtcggcaccagttgcgtgagcggaaagatggccgcttccc
ggccctgctgcagggagctcaaaatggaggacgcggcgctcgggagagcgggcgggtgagtcacccacac
aaaggaaaagggcctttccgtcctcagccgtcgcttcatgtgactccacggagtaccgggcgccgtccag
gcacctcgattagttctcgagcttttggagtacgtcgtctttaggttggggggaggggttttatgcgatg
gagtttccccacactgagtgggtggagactgaagttaggccagcttggcacttgatgtaattctccttgg
aatttgccctttttgagtttggatcttggttcattctcaagcctcagacagtggttcaaagtttttttct
tccatttcaggtgtcgtgaggatctatttccggtgaattcgccaccATGGTCCTGACGCTGCTTCTCTCC
GCCTACAAGCTGTGTCGCTTCTTCGCCATGTCGGGCCCACGGCCGGGCGCCGAGCGGGATTACAAGGACG
ACGATGACAAGCTGGCGGTGCCTGGGCCAGATGGGGGCGGTGGCACGGGCCCATGGTGGGCTGCGGGTGG
CCGCGGGCCCCGCGAAGTGTCGCCGGGGGCAGGCACCGAGGTGCAGGACGCCCTGGAGCGCGCGCTGCCG
GAGCTGCAGCAGGCCTTGTCCGCGCTGAAGCAGGCGGGCGGCGCGCGGGCCGTGGGCGCCGGCCTGGCCG
AGGTCTTCCAACTGGTGGAGGAGGCCTGGCTGCTGCCGGCCGTGGGCCGCGAGGTAGCCCAGGGTCTGTG
CGACGCCATCCGCCTCGATGGCGGCCTCGACCTGCTGTTGCGGCTGCTGCAGGCGCCGGAGTTGGAGACG
CGTGTGCAGGCCGCGCGCCTGCTGGAGCAGATCCTGGTGGCTGAGAACCGAGACCGCGTGGCGCGCATTG
GGCTGGGCGTGATCCTGAACCTGGCGAAGGAACGCGAACCCGTAGAGCTGGCGCGGAGCGTGGCAGGCAT
CTTGGAGCACATGTTCAAGCATTCGGAGGAGACATGCCAGAGGCTGGTGGCGGCCGGCGGCCTGGACGCG
GTGCTGTATTGGTGCCGCCGCACGGACCCCGCGCTGCTGCGCCACTGCGCGCTGGCGCTGGGCAACTGCG
CGCTGCACGGGGGCCAGGCGGTGCAGCGACGCATGGTAGAGAAGCGCGCAGCCGAGTGGCTCTTCCCGCT
CGCCTTCTCCAAGGAGGACGAGCTGCTTCGGCTGCACGCCTGCCTCGCAGTAGCGGTGTTGGCGACTAAC
AAGGAGGTGGAGCGCGAGGTGGAGCGCTCGGGCACGCTGGCGCTCGTGGAGCCGCTTGTGGCCTCGCTGG
ACCCTGGCCGCTTCGCCCGCTGTCTGGTGGACGCCAGCGACACAAGCCAGGGCCGCGGGCCCGACGACCT
GCAGCGCCTCGTGCCGTTGCTCGACTCTAACCGCTTGGAGGCGCAGTGCATCGGGGCTTTCTACCTCTGC
GCCGAGGCTGCCATCAAGAGCCTGCAAGGCAAGACCAAGGTGTTCAGCGACATCGGCGCCATCCAGAGCC
TGAAACGCCTGGTTTCCTACTCTACCAATGGCACTAAGTCGGCGCTGGCCAAGCGCGCGCTGCGCCTGCT
GGGCGAGGAGGTGCCACGGCCCATCCTGCCCTCCGTGCCCAGCTGGAAGGAGGCCGAGGTTCAGACGTGG
CTGCAGCAGATCGGTTTCTCCAAGTACTGCGAGAGCTTCCGGGAGCAGCAGGTGGATGGCGACCTGCTTC
TGCGGCTCACGGAGGAGGAACTCCAGACCGACCTGGGCATGAAATCGGGCATCACCCGCAAGAGGTTCTT
TAGGGAGCTCACGGAGCTCAAGACCTTCGCCAACTATTCTACGTGCGACCGCAGCAACCTGGCGGACTGG
CTGGGCAGCCTGGACCCGCGCTTCCGCCAGTACACCTACGGCCTGGTCAGCTGCGGCCTGGACCGCTCCC
TGCTGCACCGCGTGTCTGAGCAGCAGCTGCTGGAAGACTGCGGCATCCACCTGGGCGTGCACCGCGCCCG
CATCCTCACGGCGGCCAGAGAAATGCTACACTCCCCGCTGCCCTGTACTGGTGGCAAACCCAGTGGGGAC
ACTCCAGATGTCTTCATCAGCTACCGCCGGAACTCAGGTTCCCAGCTGGCCAGTCTCCTGAAGGTGCACC
TGCAGCTGCATGGCTTCAGTGTCTTCATTGATGTGGAGAAGCTGGAAGCAGGCAAGTTCGAGGACAAACT
CATCCAGAGTGTCATGGGTGCCCGCAACTTTGTGTTGGTGCTATCACCTGGAGCACTGGACAAGTGCATG
CAAGACCATGACTGCAAGGATTGGGTGCATAAGGAGATTGTGACTGCTTTAAGCTGCGGCAAGAACATTG
TGCCCATCATTGATGGCTTCGAGTGGCCTGAGCCCCAGGTCCTGCCTGAGGACATGCAGGCTGTGCTTAC
TTTCAACGGTATCAAGTGGTCCCACGAATACCAGGAGGCCACCATTGAGAAGATCATCCGCTTCCTGCAG
GGCCGCTCCTCCCGGGACTCATCTGCAGGCTCTGACACCAGTTTGGAGGGTGCTGCACCCATGGGTCCAA
CCtacccatacgatgttccagattacgctTAAggatcccgcccctctccctcccccccccctaacgttac
tggccgaagccgcttggaataaggccggtgtgcgtttgtctatatgttattttccaccatattgccgtct
tttggcaatgtgagggcccggaaacctggccctgtcttcttgacgagcattcctaggggtctttcccctc
tcgccaaaggaatgcaaggtctgttgaatgtcgtgaaggaagcagttcctctggaagcttcttgaagaca
aacaacgtctgtagcgaccctttgcaggcagcggaaccccccacctggcgacaggtgcctctgcggccaa
aagccacgtgtataagatacacctgcaaaggcggcacaaccccagtgccacgttgtgagttggatagttg
tggaaagagtcaaatggctctcctcaagcgtattcaacaaggggctgaaggatgcccagaaggtacccca
ttgtatgggatctgatctggggcctcggtgcacatgctttacatgtgtttagtcgaggttaaaaaaacgt
ctaggccccccgaaccacggggacgtggttttcctttgaaaaacacgatgataagcttgccacaacccac
aaggagacgaccttccatgaccgagtacaagcccacggtgcgcctcgccacccgcgacgacgtcccccgg
gccgtacgcaccctcgccgccgcgttcgccgactaccccgccacgcgccacaccgtcgacccggaccgcc
acatcgagcgggtcaccgagctgcaagaactcttcctcacgcgcgtcgggctcgacatcggcaaggtgtg
ggtcgcggacgacggcgccgcggtggcggtctggaccacgccggagagcgtcgaagcgggggcggtgttc
gccgagatcggcccgcgcatggccgagttgagcggttcccggctggccgcgcagcaacagatggaaggcc
tcctggcgccgcaccggcccaaggagcccgcgtggttcctggccaccgtcggcgtctcgcccgaccacca
gggcaagggtctgggcagcgccgtcgtgctccccggagtggaggcggccgagcgcgccggggtgcccgcc
ttcctggagacctccgcgccccgcaacctccccttctacgagcggctcggcttcaccgtcaccgccgacg
tcgaggtgcccgaaggaccgcgcacctggtgcatgacccgcaagcccggtgcctagacgcgtctggaaca
atcaacctctggattacaaaatttgtgaaagattgactggtattcttaactatgttgctccttttacgct
atgtggatacgctgctttaatgcctttgtatcatgctattgcttcccgtatggctttcattttctcctcc
ttgtataaatcctggttgctgtctctttatgaggagttgtggcccgttgtcaggcaacgtggcgtggtgt
gcactgtgtttgctgacgcaacccccactggttggggcattgccaccacctgtcagctcctttccgggac
tttcgctttccccctccctattgccacggcggaactcatcgccgcctgccttgcccgctgctggacaggg
gctcggctgttgggcactgacaattccgtggtgttgtcggggaagctgacgtcctttccatggctgctcg
cctgtgttgccacctggattctgcgcgggacgtccttctgctacgtcccttcggccctcaatccagcgga
ccttccttcccgcggcctgctgccggctctgcggcctcttccgcgtcttcgccttcgccctcagacgagt
cggatctccctttgggccgcctccccgcctggaattaattctgcagtcgagacctagaaaaacatggagc
aatcacaagtagcaatacagcagctaccaatgctgattgtgcctggctagaagcacaagaggaggaggag
gtgggttttccagtcacacctcaggtacctttaagaccaatgacttacaaggcagctgtagatcttagcc
actttttaaaagaaaagaggggactggaagggctaattcactcccaacgaagacaagatatccttgatct
gtggatctaccacacacaaggctacttccctgattagcagaactacacaccagggccaggggtcagatat
ccactgacctttggatggtgctacaagctagtaccagttgagccagataaggtagaagaggccaataaag
gagagaacaccagcttgttacaccctgtgagcctgcatgggatggatgacccggagagagaagtgttaga
gtggaggtttgacagccgcctagcatttcatcacgtggcccgagagctgcatccggagtacttcaagaac
tgctgatatcgagcttgctacaagggactttccgctggggactttccagggaggcgtggcctgggcggga
ctggggagtggcgagccctcagatcctgcatataagcagctgctttttgcctgtactgggtctctctggt
tagaccagatctgagcctgggagctctctggctaactagggaacccactgcttaagcctcaataaagctt
gccttgagtgcttcaagtagtgtgtgcccgtctgttgtgtgactctggtaactagagatccctcagaccc
ttttagtcagtgtggaaaatctctagcagtagtagttcatgtcatcttattattcagtatttataacttg
caaagaaatgaatatcagagagtgagaggccttgacattgctagcgtttaccgtcgacctctagctagag
cttggcgtaatcatggtcatagctgtttcctgtgtgaaattgttatccgctcacaattccacacaacata
cgagccggaagcataaagtgtaaagcctggggtgcctaatgagtgagctaactcacattaattgcgttgc
gctcactgcccgctttccagtcgggaaacctgtcgtgccagctgcattaatgaatcggccaacgcgcggg
gagaggcggtttgcgtattgggcgctcttccgcttcctcgctcactgactcgctgcgctcggtcgttcgg
ctgcggcgagcggtatcagctcactcaaaggcggtaatacggttatccacagaatcaggggataacgcag
gaaagaacatgtgagcaaaaggccagcaaaaggccaggaaccgtaaaaaggccgcgttgctggcgttttt
ccataggctccgcccccctgacgagcatcacaaaaatcgacgctcaagtcagaggtggcgaaacccgaca
ggactataaagataccaggcgtttccccctggaagctccctcgtgcgctctcctgttccgaccctgccgc
ttaccggatacctgtccgcctttctcccttcgggaagcgtggcgctttctcatagctcacgctgtaggta
tctcagttcggtgtaggtcgttcgctccaagctgggctgtgtgcacgaaccccccgttcagcccgaccgc
tgcgccttatccggtaactatcgtcttgagtccaacccggtaagacacgacttatcgccactggcagcag
ccactggtaacaggattagcagagcgaggtatgtaggcggtgctacagagttcttgaagtggtggcctaa
ctacggctacactagaagaacagtatttggtatctgcgctctgctgaagccagttaccttcggaaaaaga
gttggtagctcttgatccggcaaacaaaccaccgctggtagcggtggtttttttgtttgcaagcagcaga
ttacgcgcagaaaaaaaggatctcaagaagatcctttgatcttttctacggggtctgacgctcagtggaa
cgaaaactcacgttaagggattttggtcatgagattatcaaaaaggatcttcacctagatccttttaaat
taaaaatgaagttttaaatcaatctaaagtatatatgagtaaacttggtctgacagttaccaatgcttaa
tcagtgaggcacctatctcagcgatctgtctatttcgttcatccatagttgcctgactccccgtcgtgta
gataactacgatacgggagggcttaccatctggccccagtgctgcaatgataccgcgagacccacgctca
ccggctccagatttatcagcaataaaccagccagccggaagggccgagcgcagaagtggtcctgcaactt
tatccgcctccatccagtctattaattgttgccgggaagctagagtaagtagttcgccagttaatagttt
gcgcaacgttgttgccattgctacaggcatcgtggtgtcacgctcgtcgtttggtatggcttcattcagc
tccggttcccaacgatcaaggcgagttacatgatcccccatgttgtgcaaaaaagcggttagctccttcg
gtcctccgatcgttgtcagaagtaagttggccgcagtgttatcactcatggttatggcagcactgcataa
ttctcttactgtcatgccatccgtaagatgcttttctgtgactggtgagtactcaaccaagtcattctga
gaatagtgtatgcggcgaccgagttgctcttgcccggcgtcaatacgggataataccgcgccacatagca
gaactttaaaagtgctcatcattggaaaacgttcttcggggcgaaaactctcaaggatcttaccgctgtt
gagatccagttcgatgtaacccactcgtgcacccaactgatcttcagcatcttttactttcaccagcgtt
tctgggtgagcaaaaacaggaaggcaaaatgccgcaaaaaagggaataagggcgacacggaaatgttgaa
tactcatactcttcctttttcaatattattgaagcatttatcagggttattgtctcatgagcggatacat
atttgaatgtatttagaaaaataaacaaataggggttccgcgcacatttccccgaaaagtgccacctgac
gtcgacggatcgggagatcaacttgtttattgcagcttataatggttacaaataaagcaatagcatcaca
aatttcacaaataaagcatttttttcactgcattctagttgtggtttgtccaaactcatcaatgtatott
atcatgtctggatcaactggataactcaagctaaccaaaatcatcccaaacttcccaccccataccctat
taccactgccaattacctgtggtttcatttactctaaacctgtgattcctctgaattattttcattttaa
agaaattgtatttgttaaatatgtactacaaacttagtagt

In certain embodiments, provided is a method for determining modulation of Sterile alpha and Toll/interleukin-1 receptor motif-containing 1 (SARM1) by a candidate compound, said method comprising:
providing a solution comprising a SARM1 protein and the candidate compound;
adding to the solution nicotinamide mononucleotide (NMN) and nicotinamide adenine dinucleotide (NAD⁺); and
measuring the amount of NAD⁺ remaining in solution to determine modulation of SARM1 by the candidate compound.

In certain embodiments, the measuring comprises a fluorescent detection step.

In certain embodiments, the SARM1 protein is a protein comprising at least about 60%, or at least about 70%, or at least about 80%, or at least about 90%, or at least about 95%, or at least about 97%, or at least about 99% sequence homology to native SARM1 protein.

In certain embodiments, the SARM1 protein comprises a fluorescent tag.

In certain embodiments, the SARM1 protein is provided using SEQ. ID. 1, or a derivative thereof. In certain embodiments, the derivative comprises at least about 60%, or at least about 70%, or at least about 80%, or at least about 90%, or at least about 95%, or at least about 97%, or at least about 99% sequence homology to SEQ. ID. 1. In certain embodiments, the SARM1 protein is provided using SEQ. ID. 1.

In certain embodiments, the candidate compound is an inhibitor of SARM1.

Unless otherwise defined, all technical and scientific terms used herein have the same meaning as commonly understood by one of ordinary skill in the art to which this disclosure belongs.

The disclosure illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claims.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A compound of Formula I: 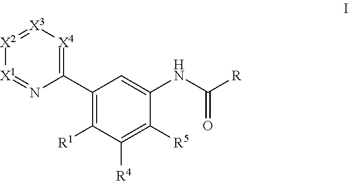
or a pharmaceutically acceptable salt, isotopically enriched analog, stereoisomer, tautomer, mixture of stereoisomers thereof, wherein:
each of X¹, X², X³, and X⁴ are independently N or CR⁶; provided no more than two of X¹, X², X³, and X⁴ are N;
R is tert-butyl, C₁₋₆ alkyl substituted with one to five R⁸, —NR²R³, —O—R⁷, C₃₋₁₀cycloalkyl, or heterocyclyl; and the C₃₋₁₀ cycloalkyl or heterocyclyl is independently optionally substituted with one to five Z¹;
R¹ is halo, cyano, —NO₂, C₁₋₆ alkyl, C₂₋₆alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C_{1_6} alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R² is C₁₋₆ alkyl, C₂₋₆alkenyl, C₂₋₆alkynyl, C₃₋₁₀ cycloalkyl, heterocyclyl, aryl, or heteroaryl; wherein the C₁₋₆ alkyl, C₂₋₆alkenyl, C₂₋₆ alkynyl, C₃₋₁₀ cycloalkyl, heterocyclyl, aryl, or heteroaryl is independently optionally substituted with one to five Z¹;
R³ is C₁₋₆ alkyl, C₂₋₆alkenyl, C₂₋₆alkynyl, C₃₋₁₀ cycloalkyl, heterocyclyl, aryl, or heteroaryl; wherein the C₁₋₆ alkyl, C₂₋₆alkenyl, C₂₋₆ alkynyl, C₃₋₁₀ cycloalkyl, heterocyclyl, aryl, or heteroaryl is independently optionally substituted with one to five Z¹;
or R² and R³ together form a heterocyclyl, which may further be independently optionally substituted with one to five Z¹;
R⁴ is hydrogen, halo, cyano, C₁₋₆ alkyl, C₂₋₆alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R⁵ is hydrogen, halo, cyano, C₁₋₆ alkyl, C₂₋₆alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁶ is independently hydrogen, halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R⁷ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁸ is independently halo, cyano, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₃₋₁₀ cycloalkyl, heterocyclyl, aryl, or heteroaryl is independently optionally substituted with one to five Z¹;
each R¹¹ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z¹ is independently halo, cyano, —NO₂, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, —N(R¹²)₂, —OR¹², —SR¹², —C(O)R¹², —C(O)OR¹², —S(O)R¹², —S(O)₂R¹², —C(O)N(R¹²)₂, —NR¹²C(O)R¹², —NR¹²S(O)R¹², —NR¹²S(O)₂R¹², —S(O)N(R¹²)₂, —S(O)₂N(R¹²)₂, —NR¹²C(O)N(R¹²)₂, —NR¹²S(O)N(R¹²)₂, —NR¹²S(O)₂N(R¹²)₂, —OC(O)N(R¹²)₂, or —NR¹²C(O)OR¹²; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with one to five Z^{1a};
each R¹² is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1a} is independently halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —SR¹³, —C(O)R¹³, —C(O)OR¹³, —S(O)R¹³, —S(O)₂R¹³, —C(O)N(R¹³)₂, —NR¹³C(O)R¹³, —NR¹³S(O)R¹³, —NR¹³S(O)₂R¹³, —S(O)N(R¹³)₂, —S(O)₂N(R¹³)₂, —NR¹³C(O)N(R¹³)₂, —NR¹³S(O)N(R¹³)₂, —NR¹³S(O)₂N(R¹³)₂, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1b} is independently halo, cyano, —OH, —SH, —NH₂, —NO₂, C₁₋₆ alkyl, C₁₋₆ haloalkyl, C₂₋₆ alkenyl, C₂₋₆ alkynyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five halo, cyano, —OH, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl;
provided that:
i) the compound is not N-[3-[6-ethoxy-5-(tetrahydro-2H-pyran-4-yl)-3-pyridazinyl]-4-methylphenyl]-2,3-dihydro-1H-isoindole-5-carboxamide, (3R)-1-[2-[4-(4-acetylphenyl)-1-piperazinyl]-2-oxoethyl]-N-[4-hydroxy-3-(2-pyridinyl)phenyl]-3-pyrrolidinecarboxamide, 2,3-dihydro-5-methoxy-N-[4-methyl-3-(2-pyridinyl)phenyl]-6-(trifluoromethyl)-1H-indole-1-carboxamide, N-[3-[3-chloro-5-(trifluoromethyl)-2-pyridinyl]-4-methoxyphenyl]hexahydro-1,3-dimethyl-4,6-dioxo-2-thioxo-5-pyrimidinecarboxamide, (3R,5aS,6R,8aS,9S,11R,11aR)—N-[4-chloro-3-(2-pyridinyl)phenyl]octahydro-3,6,9-trimethyl-3,11-epoxy-3H,11H-furo[3,4-j]-1,2-benzodioxepin-9-carboxamide, 1-(1,3-benzodioxol-5-yl)-N-[4-methyl-3-(2-pyridinyl)phenyl]-cyclopropanecarboxamide, N-[4-[(4-propyl-1-piperazinyl)carbonyl]-3-(2-pyridinyl)phenyl]-cyclopropanecarboxamide, dibutyl 4,4′-(((2-(pyridin-2-yl)-1,4-phenylene)bis(azanediyl))bis(carbonyl))bis(cyclohexane-1-carboxylate), N-[3-(2-amino-4-pyrimidinyl)-4-fluorophenyl]-1-pyrrolidinecarboxamide, N-[3-(3-amino-1,2,4-triazin-5-yl)-4-fluorophenyl]-1-pyrrolidinecarboxamide, 2-[4-methyl-6-(4-morpholinyl)-1,3,5-triazin-2-yl]-4-[(4-morpholinylcarbonyl)amino]benzenesulfonic acid, N-[4-fluoro-3-[2-[3-(hydroxymethyl)phenyl]-6-(4-morpholinyl)-4-pyrimidinyl]phenyl]-1-piperazinecarboxamide, 2,3-dihydro-5-methoxy-N-[4-methyl-3-(2-pyridinyl)phenyl]-6-(trifluoromethyl)-1H-indole-1-carboxamide, rel-N-[3,4-difluoro-5-[6-[[(3aR,6aS)-octahydro-2-[(tetrahydro-2H-pyran-4-yl)methyl]cyclopenta[c]pyrrol-5-yl]amino]-3-pyridazinyl]phenyl]-1-hydroxycyclobutanecarboxamide, rel-N-[3,4-difluoro-5-[6-[[(3aR,6aS)-octahydro-2-[(tetrahydro-2H-pyran-4-yl)methyl]cyclopenta[c]pyrrol-5-yl]amino]-3-pyridazinyl]phenyl]-3,3-difluorocyclobutanecarboxamide, rel-N-[3,4-difluoro-5-[6-[[(3aR,6aS)-octahydro-2-[(tetrahydro-2H-pyran-4-yl)methyl]cyclopenta[c]pyrrol-5-yl]amino]-3-pyridazinyl]phenyl]-1-fluorocyclopropanecarboxamide, or rel-N-[3,4-difluoro-5-[6-[[(3aR,6aS)-octahydro-2-[(tetrahydro-2H-pyran-4-yl)methyl]cyclopenta[c]pyrrol-5-yl]amino]-3-pyridazinyl]phenyl]-1-(trifluoromethyl)cyclopropanecarboxamide;
ii) when X² is N, R¹ is methyl, R⁴ is hydrogen or fluoro, and R⁵ is hydrogen, then neither of X¹ and X³ are C-morpholino;
iii) when R is —O—R⁷, then R¹ is methyl;
iv) when R is substituted C₁₋₆ alkyl, then R¹ is substituted or unsubstituted C₁₋₆ alkyl; and
v) when R is substituted or unsubstituted heterocyclyl, the substituted or unsubstituted heterocyclyl is other than a substituted or unsubstituted pyridin-2(1H)-onyl.

2. The compound of claim 1, wherein R is tert-butyl, C₁₋₆ alkyl, C₃₋₁₀ cycloalkyl, or heterocyclyl; wherein the C₁₋₆ alkyl is substituted with one to five R⁸, and the C₃₋₁₀ cycloalkyl or heterocyclyl is independently optionally substituted with one to five Z¹.

3. The compound of claim 1, represented by Formula II:

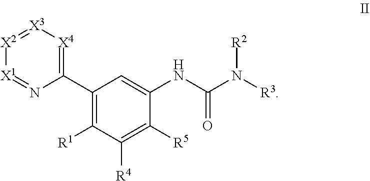

4. The compound of claim 1, represented by Formula III:

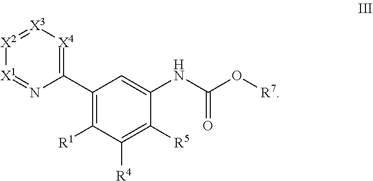

5. The compound of any preceding claim, wherein X¹ is N.

6. The compound of any preceding claim, wherein X², X³, and X⁴ are CR⁶.

7. The compound of any one of claims 1-4, wherein X² is N.

8. The compound of claim 7, wherein X¹, X³, and X⁴ are CR⁶.

9. The compound of any one of claims 1-4, wherein X³ is N.

10. The compound of claim 9, wherein X¹, X², and X⁴ are CR⁶.

11. The compound of claim 9, wherein X¹ is N and X² and X⁴ are CR⁶.

12. The compound of any one of claims 1-4, wherein X⁴ is N.

13. The compound of claim 11, wherein X¹, X², and X³ are CR⁶.

14. The compound of claim 12, wherein X¹ is N and X² and X³ are CR⁶.

15. The compound of claim 12, wherein X² is N and X¹ and X³ are CR⁶.

16. The compound of claim 12, wherein X³ is N and X¹ and X² are CR⁶.

17. A compound of Formula IV: 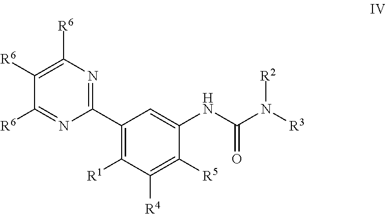
or a pharmaceutically acceptable salt, isotopically enriched analog, stereoisomer, tautomer, mixture of stereoisomers thereof, wherein:
R¹ is hydrogen, halo, cyano, —NO₂, C₁₋₆ alkyl, C₂₋₆alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆alkynyl, C₃₋₁₀ cycloalkyl, heterocyclyl, aryl, or heteroaryl, is independently optionally substituted with one to five Z¹;
R² is C₁₋₆ alkyl, C₂₋₆alkenyl, C₂₋₆alkynyl, C₃₋₁₀ cycloalkyl, heterocyclyl, aryl, or heteroaryl; wherein the C₁₋₆ alkyl, C₂₋₆alkenyl, C₂₋₆ alkynyl, C₃₋₁₀ cycloalkyl, heterocyclyl, aryl, or heteroaryl is independently optionally substituted with one to five Z¹;
R³ is C₁₋₆ alkyl, C₂₋₆alkenyl, C₂₋₆alkynyl, C₃₋₁₀ cycloalkyl, heterocyclyl, aryl, or heteroaryl; wherein the C₁₋₆ alkyl, C₂₋₆alkenyl, C₂₋₆ alkynyl, C₃₋₁₀ cycloalkyl, heterocyclyl, aryl, or heteroaryl is independently optionally substituted with one to five Z¹;
or R² and R³ together form a heterocyclyl, which may further be independently optionally substituted with one to five Z¹;
R⁴ is hydrogen, halo, cyano, C₁₋₆ alkyl, C₂₋₆alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R⁵ is hydrogen, halo, cyano, C₁₋₆ alkyl, C₂₋₆alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁶ is independently hydrogen, halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
each R¹¹ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z¹ is independently halo, cyano, —NO₂, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, —N(R¹²)₂, —OR¹², —SR¹², —C(O)R¹², —C(O)OR¹², —S(O)R¹², —S(O)₂R¹², —C(O)N(R¹²)₂, —NR¹²C(O)R¹², —NR¹²S(O)R¹², —NR¹²S(O)₂R¹², —S(O)N(R¹²)₂, —S(O)₂N(R¹²)₂, —NR¹²C(O)N(R¹²)₂, —NR¹²S(O)N(R¹²)₂, —NR¹²S(O)₂N(R¹²)₂, —OC(O)N(R¹²)₂, or —NR¹²C(O)OR¹²; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with one to five Z^{1a};
each R¹² is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1a} is independently halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —SR¹³, —C(O)R¹³, —C(O)OR¹³, —S(O)R¹³, —S(O)₂R¹³, —C(O)N(R¹³)₂, —NR¹³C(O)R¹³, —NR¹³S(O)R¹³, —NR¹³S(O)₂R¹³, —S(O)N(R¹³)₂, —S(O)₂N(R¹³)₂, —NR¹³C(O)N(R¹³)₂, —NR¹³S(O)N(R¹³)₂, —NR¹³S(O)₂N(R¹³)₂, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1b} is independently halo, cyano, —OH, —SH, —NH₂, —NO₂, C₁₋₆ alkyl, C₁₋₆ haloalkyl, C₂₋₆ alkenyl, C₂₋₆ alkynyl, C₃₋₁₀ cycloalkyl, heterocyclyl, aryl, heteroaryl, -L-C₁₋₆ alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁₋₆ haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five halo, cyano, —OH, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl;
provided that:
a) the moiety 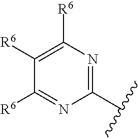
is not 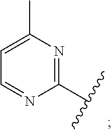 [SMILES: *c1nccc(C)n1]
and
b) the compound is not N-[3-[4-[3-(hydroxymethyl)phenyl]-6-(4-morpholinyl)-2-pyrimidinyl]phenyl]-1-piperazinecarboxamide, N-[3-[4-(1H-indazol-5-ylamino)-2-pyrimidinyl]phenyl]-4-morpholinecarboxamide, N-[3-ethyl-5-(2-pyrimidinyl)phenyl]-7,8-dihydro-4-(2-methylphenyl)-2-(3-pyridinyl)pyrido[4,3-d]pyrimidine-6(5H)-carboxamide, or 1,1-dimethylethyl 5-[[(1,1-dimethylethoxy)carbonyl][2-[3-[(4-morpholinylcarbonyl)amino]phenyl]-4-pyrimidinyl]amino]-1H-indazole-1-carboxylate.

18. The compound of any preceding claim, wherein R⁴ is hydrogen, fluoro, chloro, bromo, cyano, or methyl.

19. The compound of any preceding claim, wherein R⁵ is hydrogen, fluoro, or chloro.

20. The compound of any preceding claim, wherein R⁴ and R⁵ are hydrogen.

21. The compound of claim 1, represented by Formula IA:

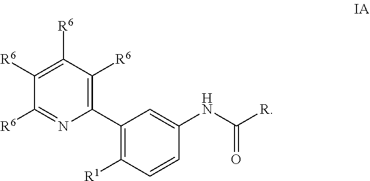

22. The compound of claim 1, represented by Formula IIA:

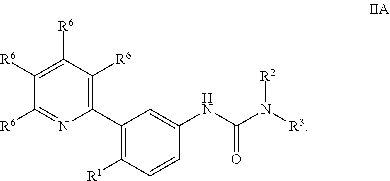

23. The compound of claim 1, represented by Formula IIIA:

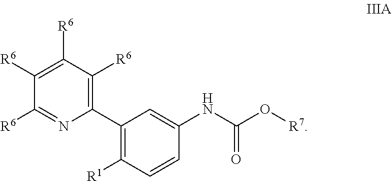

24. The compound of any preceding claim, wherein each R⁶ is independently hydrogen, halo, cyano, C₁₋₆ alkyl, C₁₋₆haloalkyl, C₃₋₁₀ cycloalkyl, —O—C₁₋₆ alkyl, or —O—C₁₋₆ haloalkyl.

25. The compound of any one of claims 1-23, wherein each R⁶ is independently hydrogen, fluoro, chloro, cyano, methyl, ethyl, trifluoromethyl, cyclopropyl, methoxy, 2-methoxyethoxymethyl, fluoromethoxy, or difluoromethoxy.

26. The compound of any preceding claim, wherein R¹ is halo, cyano, C₁₋₆ alkyl, C₃₋₁₀ cycloalkyl, aryl, or —OR¹¹; wherein the C₁₋₆ alkyl, C₃₋₁₀ cycloalkyl, or aryl is optionally substituted with one to five Z¹.

27. The compound of any preceding claim, wherein R¹ is fluoro, chloro, bromo, cyano, methyl, ethyl, isopropyl, —CH₂F, —CHF₂, —CF₃, —CH₂CN, —OCF₃, cyclopropyl optionally substituted with methyl, or phenyl.

28. The compound of any one of claims 1, 3, 17, or 22, wherein R² and R³ together form a heterocyclyl, which may further be independently optionally substituted with one to five Z¹.

29. The compound of claim 28, wherein R² and R³ together form a heterocyclyl, which may further be independently optionally substituted with halo, —OH, —C(O)O—C₁₋₁₆ alkyl, —O—C₁₋₁₆ alkyl, —O—C₁₋₆ haloalkyl, C₁₋₆ alkyl, C₁₋₆ haloalkyl, C₃₋₁₀ cycloalkyl, aryl, or heteroaryl; wherein the C₁₋₆ alkyl or heteroaryl is optionally substituted with one to five Z¹.

30. The compound of any one of claims 1, 3, 22, or 28-29, wherein R or the moiety 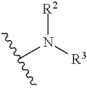
is:

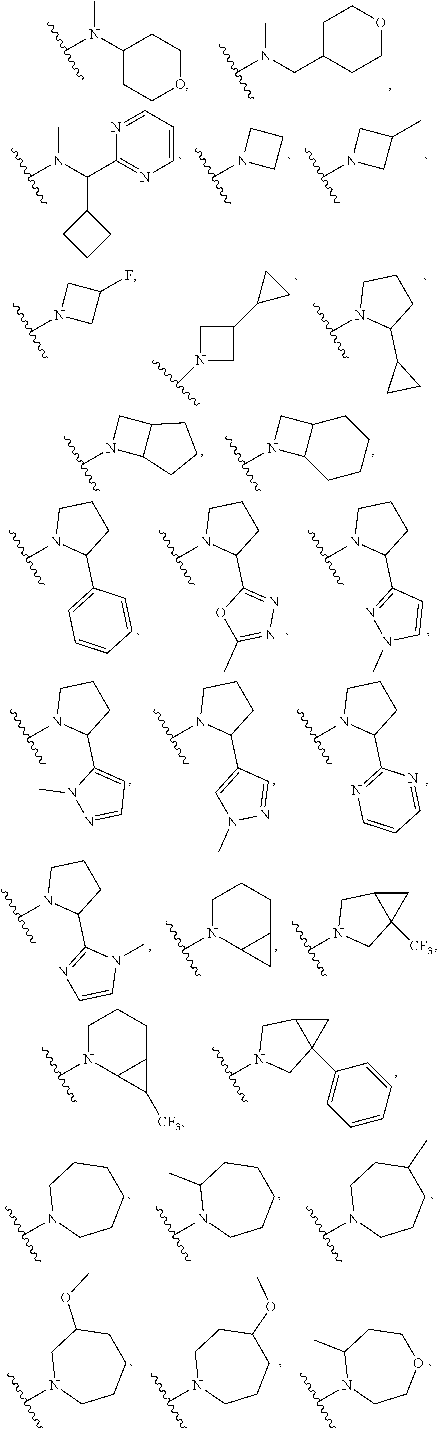

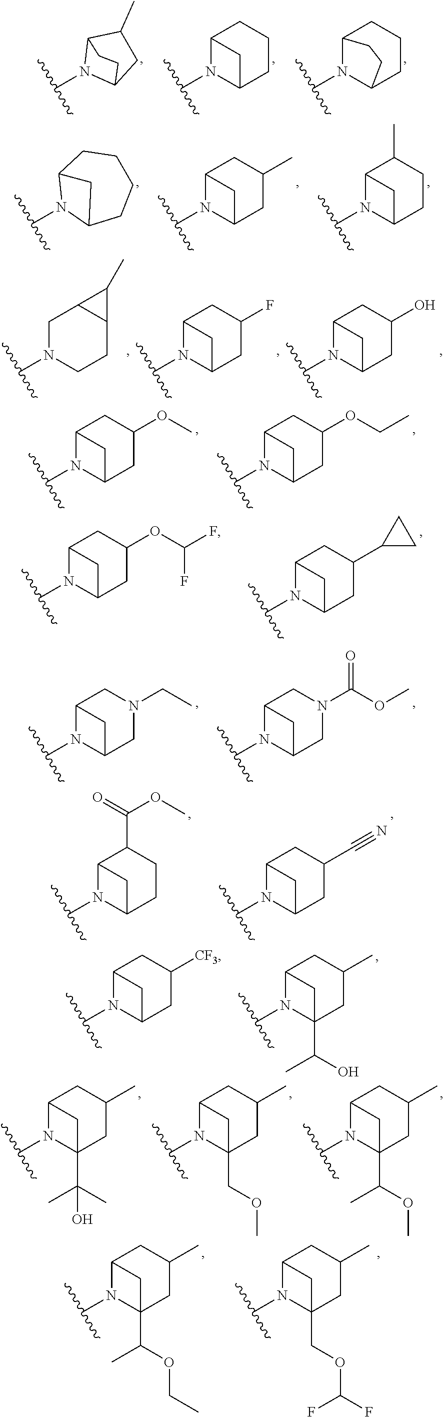

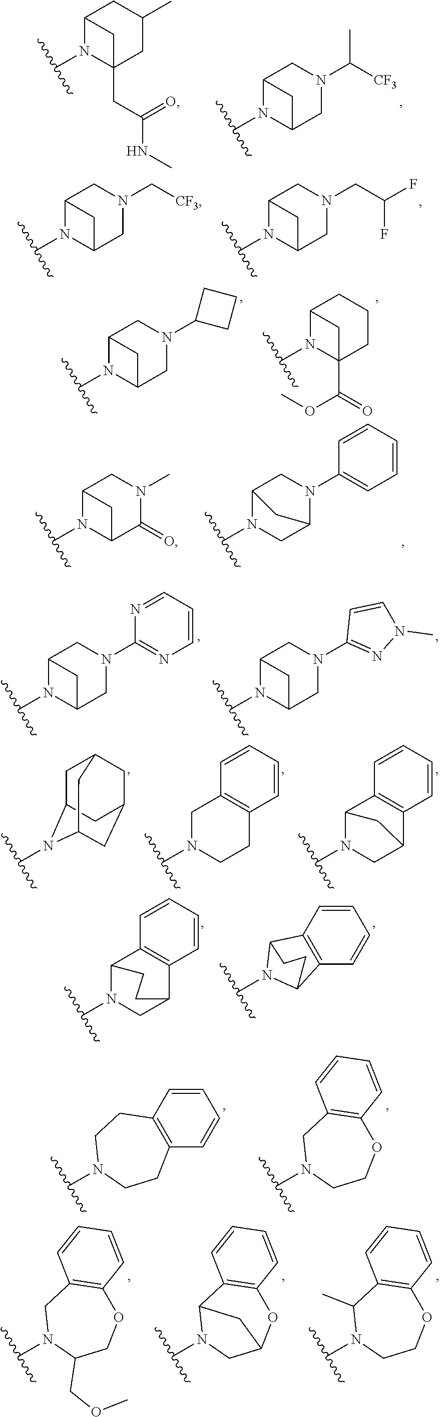

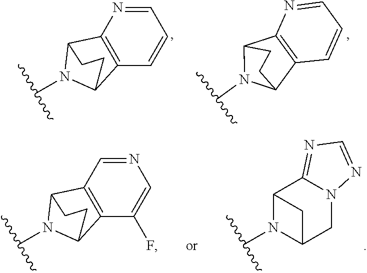 [SMILES: *N1C2CCC1c1ncccc12 ; *N1C2CCC1c1ncccc12 ; *N1C2CCC1c1c(F)cncc12 ; *N1C2CC1c1ncnn1C2]

31. A compound selected from Table 1, or a pharmaceutically acceptable salt thereof.

32. A compound selected from Table 2, or a pharmaceutically acceptable salt thereof.

33. A pharmaceutical composition comprising a compound of any preceding claim, or a pharmaceutically acceptable salt, isotopically enriched analog, stereoisomer, tautomer, or mixture of stereoisomers thereof, and a pharmaceutically acceptable carrier.

34. A method for inhibiting SARM1 activity, the method comprising contacting a cell with an effective amount of a compound of any one of claims 1-32, or a pharmaceutically acceptable salt, isotopically enriched analog, stereoisomer, tautomer, or mixture of stereoisomers thereof, the pharmaceutical composition of claim 33; or a compound of Formula I: 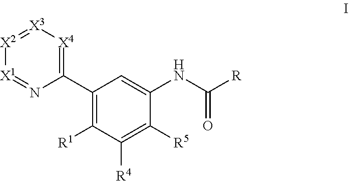
or a pharmaceutically acceptable salt, isotopically enriched analog, stereoisomer, tautomer, mixture of stereoisomers thereof, wherein:
each of X¹, X², X³, and X⁴ are independently N or CR⁶; provided no more than two of X¹, X², X³, and X⁴ are N;
R is C₁₋₆ alkyl, —NR²R³, —O—R⁷, C₃₋₁₀ cycloalkyl, heterocyclyl, aryl, or heteroaryl; wherein the C₁₋₆ alkyl, C₃₋₁₀ cycloalkyl, heterocyclyl, aryl, or heteroaryl is independently optionally substituted with one to five Z¹;
R¹ is hydrogen, halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R² is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R³ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
or R² and R³ together form a heterocyclyl, which may further be independently optionally substituted with one to five Z¹;
R⁴ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
R⁵ is hydrogen, halo, cyano, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁶ is independently hydrogen, halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein each C₁₋₆ alkyl, C₂₋₆ alkenyl, C₂₋₆ alkynyl, C₃₋₁₀ cycloalkyl, heterocyclyl, aryl, or heteroaryl, is independently optionally substituted with one to five Z¹;
R⁷ is C₁₋₆ alkyl, C₂₋₆ alkenyl, C₂₋₆ alkynyl, C₃₋₁₀ cycloalkyl, heterocyclyl, aryl, or heteroaryl; wherein the C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z¹;
each R⁸ is independently halo, cyano, C₃₋₁₀ cycloalkyl, heterocyclyl, aryl, heteroaryl, —N(R¹¹)₂, —OR¹¹, —SR¹¹, —C(O)R¹¹, —C(O)OR¹¹, —S(O)R¹¹, —S(O)₂R¹¹, —C(O)N(R¹¹)₂, —NR¹¹C(O)R¹¹, —NR¹¹S(O)R¹¹, —NR¹¹S(O)₂R¹¹, —S(O)N(R¹¹)₂, —S(O)₂N(R¹¹)₂, —NR¹¹C(O)N(R¹¹)₂, —NR¹¹S(O)N(R¹¹)₂, —NR¹¹S(O)₂N(R¹¹)₂, —OC(O)N(R¹¹)₂, or —NR¹¹C(O)OR¹¹; wherein the C₃₋₁₀ cycloalkyl, heterocyclyl, aryl, or heteroaryl is independently optionally substituted with one to five Z¹;
each R¹¹ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1a};
each Z¹ is independently halo, cyano, —NO₂, C₁₋₆ alkyl, C₂₋₆ alkenyl, C₂₋₆ alkynyl, C₁₋₆ haloalkyl, C₃₋₁₀ cycloalkyl, heterocyclyl, aryl, heteroaryl, —N(R¹²)₂, —OR¹², —SR¹², —C(O)R¹², —C(O)OR¹², —S(O)R¹², —S(O)₂R¹², —C(O)N(R¹²)₂, —NR¹²C(O)R¹², —NR¹²S(O)R¹², —NR¹²S(O)₂R¹², —S(O)N(R¹²)₂, —S(O)₂N(R¹²)₂, —NR¹²C(O)N(R¹²)₂, —NR¹²S(O)N(R¹²)₂, —NR¹²S(O)₂N(R¹²)₂, —OC(O)N(R¹²)₂, or —NR¹²C(O)OR¹²; wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, or heteroaryl is independently optionally substituted with one to five Z^{1a};
each R¹² is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1a} is independently halo, cyano, —NO₂, C₁₋₆ alkyl, C₂₋₆ alkenyl, C₂₋₆ alkynyl, C₃₋₁₀ cycloalkyl, heterocyclyl, aryl, heteroaryl, —N(R¹³)₂, —OR¹³, —SR¹³, —C(O)R¹³, —C(O)OR¹³, —S(O)R¹³, —S(O)₂R¹³, —C(O)N(R¹³)₂, —NR¹³C(O)R¹³, —NR¹³S(O)R¹³, —NR¹³S(O)₂R¹³, —S(O)N(R¹³)₂, —S(O)₂N(R¹³)₂, —NR¹³C(O)N(R¹³)₂, —NR¹³S(O)N(R¹³)₂, —NR¹³S(O)₂N(R¹³)₂, —OC(O)N(R¹³)₂, or —NR¹³C(O)OR¹³; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each R¹³ is independently hydrogen, C₁₋₆ alkyl, C₂₋₆ alkenyl, C₂₋₆ alkynyl, C₃₋₁₀ cycloalkyl, heterocyclyl, aryl, or heteroaryl; wherein each C₁₋₆ alkyl, C₂₋₆ alkenyl, C₂₋₆ alkynyl, C₃₋₁₀ cycloalkyl, heterocyclyl, aryl, or heteroaryl is independently optionally substituted with one to five Z^{1b};
each Z^{1b} is independently halo, cyano, —OH, —SH, —NH₂, —NO₂, C₁₋₆ alkyl, C₁₋₆ haloalkyl, C₂₋₆ alkenyl, C₂₋₆ alkynyl, C₃₋₁₀ cycloalkyl, heterocyclyl, aryl, heteroaryl, -L-C₁_alkyl, -L-C₂₋₆ alkenyl, -L-C₂₋₆ alkynyl, -L-C₁₋₆ haloalkyl, -L-C₃₋₁₀ cycloalkyl, -L-heterocyclyl, -L-aryl, or -L-heteroaryl; and
each L is independently —O—, —NH—, —S—, —S(O)—, —S(O)₂—, —N(C₁₋₆ alkyl)-, —N(C₂₋₆ alkenyl)-, —N(C₂₋₆ alkynyl)-, —N(C₁₋₆ haloalkyl)-, —N(C₃₋₁₀ cycloalkyl)-, —N(heterocyclyl)-, —N(aryl)-, —N(heteroaryl)-, —C(O)—, —C(O)O—, —C(O)NH—, —C(O)N(C₁₋₆ alkyl)-, —C(O)N(C₂₋₆ alkenyl)-, —C(O)N(C₂₋₆ alkynyl)-, —C(O)N(C₁_haloalkyl)-, —C(O)N(C₃₋₁₀ cycloalkyl)-, —C(O)N(heterocyclyl)-, —C(O)N(aryl)-, —C(O)N(heteroaryl)-, —NHC(O)—, —NHC(O)O—, —NHC(O)NH—, —NHS(O)—, or —S(O)₂NH—;
wherein each C₁₋₆ alkyl, C₂₋₆ alkenyl, C₂₋₆ alkynyl, C₁₋₆ haloalkyl, C₃₋₁₀ cycloalkyl, heterocyclyl, aryl, and heteroaryl of Z^{1b} and L is further independently optionally substituted with one to five halo, cyano, —OH, —SH, —NH₂, —NO₂, —SF₅, C₁₋₆ alkyl, C₂₋₆ alkenyl, C₂₋₆ alkynyl, C₁₋₆ haloalkyl, C₁₋₆ alkoxy, C₁₋₆ haloalkoxy, C₃₋₁₀ cycloalkyl, heterocyclyl, aryl, or heteroaryl.

35. The method of claim 34, wherein the contacting is in vivo.

36. A method for treating a disease or condition mediated, at least in part, by SARM1, the method comprising administering to a subject in need thereof, an effective amount of a compound of any one of claims 1-32, or a pharmaceutically acceptable salt, isotopically enriched analog, stereoisomer, tautomer, or mixture of stereoisomers thereof, or the pharmaceutical composition of claim 33.

37. A method for inhibiting axon degeneration, the method comprising administering to a subject in need thereof, an effective amount of a compound of any one of claims 1-32, or a pharmaceutically acceptable salt, isotopically enriched analog, stereoisomer, tautomer, or mixture of stereoisomers thereof, or the pharmaceutical composition of claim 33.

38. A method for treating a neurodegenerative or neurological disease or disorder, the method comprising administering an effective amount of a compound of any one of claims 1-32, or a pharmaceutically acceptable salt, isotopically enriched analog, stereoisomer, tautomer, or mixture of stereoisomers thereof, or the pharmaceutical composition of claim 33, to a subject in need thereof.

39. The method of claim 38, wherein the neurodegenerative or neurological disease or disorder is associated with axonal degeneration, axonal damage, axonopathy, a demyelinating disease, a central pontine myelinolysis, a nerve injury disease or disorder, a metabolic disease, a mitochondrial disease, metabolic axonal degeneration, axonal damage resulting from traumatic axonal injury (TAI), a leukoencephalopathy or a leukodystrophy.

40. The method of claim 36, wherein the disease or condition is a spinal cord injury, stroke, multiple sclerosis, progressive multifocal leukoencephalopathy, congenital hypomyelination, encephalomyelitis, acute disseminated encephalomyelitis, central pontine myelolysis, osmotic hyponatremia, hypoxic demyelination, ischemic demyelination, adrenoleukodystrophy, Alexander's disease, Niemann-Pick disease, Pelizaeus Merzbacher disease, periventricular leukomalacia, globoid cell leukodystrophy (Krabbe's disease), Wallerian degeneration, optic neuritis, transverse myelitis, amyotrophic lateral sclerosis (ALS, Lou Gehrig's disease), Huntington's disease, Alzheimer's disease, Parkinson's disease, Tay-Sacks disease, Gaucher's disease, Hurler Syndrome, traumatic brain injury (TBI), traumatic axonal injury (TAI), post radiation injury, neurologic complications of chemotherapy (chemotherapy induced peripheral neuropathy, CIPN), neuropathy, acute ischemic optic neuropathy, vitamin B₁₂ deficiency, isolated vitamin E deficiency syndrome, Bassen-Kornzweig syndrome, retinal degeneration, retinitis pigmentosa, glaucoma, retinitis pigmentosa, traumatic optic injury, Leber's hereditary optic atrophy (neuropathy), Leber congenital amaurosis, neuromyelitis optica, metachromatic leukodystrophy, acute hemorrhagic leukoencephalitis, trigeminal neuralgia, Bell's palsy, cerebral ischemia, multiple system atrophy, traumatic glaucoma, tropical spastic paraparesis human T-lymphotropic virus 1 (HTLV-1) associated myelopathy, west Nile virus encephalopathy, La Crosse virus encephalitis, Bunyavirus encephalitis, pediatric viral encephalitis, essential tremor, Charcot-Marie-Tooth disease, motoneuron disease, spinal muscular atrophy (SMA), hereditary sensory and autonomic neuropathy (HSAN), adrenomyeloneuropathy, progressive supra nuclear palsy (PSP), Friedrich's ataxia, hereditary ataxias, noise induced hearing loss, congenital hearing loss, Lewy Body Dementia, frontotemporal dementia, amyloidosis, diabetic neuropathy, HIV neuropathy, enteric neuropathies and axonopathies, Guillain-Barre syndrome, or severe acute motor axonal neuropathy (AMAN).

41. Use of a compound of any one of claims 1-32, or a pharmaceutically acceptable salt, isotopically enriched analog, stereoisomer, tautomer, or mixture of stereoisomers thereof, or the pharmaceutical composition of claim 33, for treating a disease or condition mediated, at least in part, by SARM1.

42. The use of claim 41, wherein the disease or condition is a spinal cord injury, stroke, multiple sclerosis, progressive multifocal leukoencephalopathy, congenital hypomyelination, encephalomyelitis, acute disseminated encephalomyelitis, central pontine myelolysis, osmotic hyponatremia, hypoxic demyelination, ischemic demyelination, adrenoleukodystrophy, Alexander's disease, Niemann-Pick disease, Pelizaeus Merzbacher disease, periventricular leukomalacia, globoid cell leukodystrophy (Krabbe's disease), Wallerian degeneration, optic neuritis, transverse myelitis, amyotrophic lateral sclerosis (ALS, Lou Gehrig's disease), Huntington's disease, Alzheimer's disease, Parkinson's disease, Tay-Sacks disease, Gaucher's disease, Hurler Syndrome, traumatic brain injury (TBI), traumatic axonal injury (TAI), post radiation injury, neurologic complications of chemotherapy (chemotherapy induced peripheral neuropathy, CIPN), neuropathy, acute ischemic optic neuropathy, vitamin B₁₂ deficiency, isolated vitamin E deficiency syndrome, Bassen-Kornzweig syndrome, retinal degeneration, retinitis pigmentosa, glaucoma, traumatic optic injury, Leber's hereditary optic atrophy (neuropathy), Leber congenital amaurosis, neuromyelitis optica, metachromatic leukodystrophy, acute hemorrhagic leukoencephalitis, trigeminal neuralgia, Bell's palsy, cerebral ischemia, multiple system atrophy, traumatic glaucoma, tropical spastic paraparesis human T-lymphotropic virus 1 (HTLV-1) associated myelopathy, west Nile virus encephalopathy, La Crosse virus encephalitis, Bunyavirus encephalitis, pediatric viral encephalitis, essential tremor, Charcot-Marie-Tooth disease, motoneuron disease, spinal muscular atrophy (SMA), hereditary sensory and autonomic neuropathy (HSAN), adrenomyeloneuropathy, progressive supra nuclear palsy (PSP), Friedrich's ataxia, hereditary ataxias, noise induced hearing loss, congenital hearing loss, Lewy Body Dementia, frontotemporal dementia, amyloidosis, diabetic neuropathy, HIV neuropathy, enteric neuropathies and axonopathies, Guillain-Barre syndrome, or severe acute motor axonal neuropathy (AMAN).

43. A compound of any one of claims 1-32, or a pharmaceutically acceptable salt, isotopically enriched analog, stereoisomer, tautomer, or mixture of stereoisomers thereof, or the pharmaceutical composition of claim 33, for use in therapy.

44. A compound of any one of claims 1-32, or a pharmaceutically acceptable salt, isotopically enriched analog, stereoisomer, tautomer, or mixture of stereoisomers thereof, or the pharmaceutical composition of claim 33, for use in treating a spinal cord injury, stroke, multiple sclerosis, progressive multifocal leukoencephalopathy, congenital hypomyelination, encephalomyelitis, acute disseminated encephalomyelitis, central pontine myelolysis, osmotic hyponatremia, hypoxic demyelination, ischemic demyelination, adrenoleukodystrophy, Alexander's disease, Niemann-Pick disease, Pelizaeus Merzbacher disease, periventricular leukomalacia, globoid cell leukodystrophy (Krabbe's disease), Wallerian degeneration, optic neuritis, transverse myelitis, amyotrophic lateral sclerosis (ALS, Lou Gehrig's disease), Huntington's disease, Alzheimer's disease, Parkinson's disease, Tay-Sacks disease, Gaucher's disease, Hurler Syndrome, traumatic brain injury (TBI), traumatic axonal injury (TAI), post radiation injury, neurologic complications of chemotherapy (chemotherapy induced peripheral neuropathy, CIPN), neuropathy, acute ischemic optic neuropathy, vitamin B₁₂ deficiency, isolated vitamin E deficiency syndrome, Bassen-Kornzweig syndrome, retinal degeneration, retinitis pigmentosa, glaucoma, traumatic optic injury, Leber's hereditary optic atrophy (neuropathy), Leber congenital amaurosis, neuromyelitis optica, metachromatic leukodystrophy, acute hemorrhagic leukoencephalitis, trigeminal neuralgia, Bell's palsy, cerebral ischemia, multiple system atrophy, traumatic glaucoma, tropical spastic paraparesis human T-lymphotropic virus 1 (HTLV-1) associated myelopathy, west Nile virus encephalopathy, La Crosse virus encephalitis, Bunyavirus encephalitis, pediatric viral encephalitis, essential tremor, Charcot-Marie-Tooth disease, motoneuron disease, spinal muscular atrophy (SMA), hereditary sensory and autonomic neuropathy (HSAN), adrenomyeloneuropathy, progressive supra nuclear palsy (PSP), Friedrich's ataxia, hereditary ataxias, noise induced hearing loss, congenital hearing loss, Lewy Body Dementia, frontotemporal dementia, amyloidosis, diabetic neuropathy, HIV neuropathy, enteric neuropathies and axonopathies, Guillain-Barre syndrome, or severe acute motor axonal neuropathy (AMAN).

45. The use of a compound of any one of claims 1-32, or a pharmaceutically acceptable salt, isotopically enriched analog, stereoisomer, tautomer, or mixture of stereoisomers thereof, or the pharmaceutical composition of claim 33, for the manufacture of a medicament for treating a spinal cord injury, stroke, multiple sclerosis, progressive multifocal leukoencephalopathy, congenital hypomyelination, encephalomyelitis, acute disseminated encephalomyelitis, central pontine myelolysis, osmotic hyponatremia, hypoxic demyelination, ischemic demyelination, adrenoleukodystrophy, Alexander's disease, Niemann-Pick disease, Pelizaeus Merzbacher disease, periventricular leukomalacia, globoid cell leukodystrophy (Krabbe's disease), Wallerian degeneration, optic neuritis, transverse myelitis, amyotrophic lateral sclerosis (ALS, Lou Gehrig's disease), Huntington's disease, Alzheimer's disease, Parkinson's disease, Tay-Sacks disease, Gaucher's disease, Hurler Syndrome, traumatic brain injury (TBI), traumatic axonal injury (TAI), post radiation injury, neurologic complications of chemotherapy (chemotherapy induced peripheral neuropathy, CIPN), neuropathy, acute ischemic optic neuropathy, vitamin B₁₂ deficiency, isolated vitamin E deficiency syndrome, Bassen-Kornzweig syndrome, retinal degeneration, retinitis pigmentosa, glaucoma, traumatic optic injury, Leber's hereditary optic atrophy (neuropathy), Leber congenital amaurosis, neuromyelitis optica, metachromatic leukodystrophy, acute hemorrhagic leukoencephalitis, trigeminal neuralgia, Bell's palsy, cerebral ischemia, multiple system atrophy, traumatic glaucoma, tropical spastic paraparesis human T-lymphotropic virus 1 (HTLV-1) associated myelopathy, west Nile virus encephalopathy, La Crosse virus encephalitis, Bunyavirus encephalitis, pediatric viral encephalitis, essential tremor, Charcot-Marie-Tooth disease, motoneuron disease, spinal muscular atrophy (SMA), hereditary sensory and autonomic neuropathy (HSAN), adrenomyeloneuropathy, progressive supra nuclear palsy (PSP), Friedrich's ataxia, hereditary ataxias, noise induced hearing loss, congenital hearing loss, Lewy Body Dementia, frontotemporal dementia, amyloidosis, diabetic neuropathy, HIV neuropathy, enteric neuropathies and axonopathies, Guillain-Barre syndrome, or severe acute motor axonal neuropathy (AMAN).


SEQUENCE LISTING (1 sequence)

SEQ ID NO: 1 - DNA, 11031 bp, synthetic construct
  source 1..11031: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgcctttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggatctatttccggtgaattcgccaccatggtcctgacgctgcttctctccgcctacaagctgtgtcgcttcttcgccatgtcgggcccacggccgggcgccgagcgggattacaaggacgacgatgacaagctggcggtgcctgggccagatgggggcggtggcacgggcccatggtgggctgcgggtggccgcgggccccgcgaagtgtcgccgggggcaggcaccgaggtgcaggacgccctggagcgcgcgctgccggagctgcagcaggccttgtccgcgctgaagcaggcgggcggcgcgcgggccgtgggcgccggcctggccgaggtcttccaactggtggaggaggcctggctgctgccggccgtgggccgcgaggtagcccagggtctgtgcgacgccatccgcctcgatggcggcctcgacctgctgttgcggctgctgcaggcgccggagttggagacgcgtgtgcaggccgcgcgcctgctggagcagatcctggtggctgagaaccgagaccgcgtggcgcgcattgggctgggcgtgatcctgaacctggcgaaggaacgcgaacccgtagagctggcgcggagcgtggcaggcatcttggagcacatgttcaagcattcggaggagacatgccagaggctggtggcggccggcggcctggacgcggtgctgtattggtgccgccgcacggaccccgcgctgctgcgccactgcgcgctggcgctgggcaactgcgcgctgcacgggggccaggcggtgcagcgacgcatggtagagaagcgcgcagccgagtggctcttcccgctcgccttctccaaggaggacgagctgcttcggctgcacgcctgcctcgcagtagcggtgttggcgactaacaaggaggtggagcgcgaggtggagcgctcgggcacgctggcgctcgtggagccgcttgtggcctcgctggaccctggccgcttcgcccgctgtctggtggacgccagcgacacaagccagggccgcgggcccgacgacctgcagcgcctcgtgccgttgctcgactctaaccgcttggaggcgcagtgcatcggggctttctacctctgcgccgaggctgccatcaagagcctgcaaggcaagaccaaggtgttcagcgacatcggcgccatccagagcctgaaacgcctggtttcctactctaccaatggcactaagtcggcgctggccaagcgcgcgctgcgcctgctgggcgaggaggtgccacggcccatcctgccctccgtgcccagctggaaggaggccgaggttcagacgtggctgcagcagatcggtttctccaagtactgcgagagcttccgggagcagcaggtggatggcgacctgcttctgcggctcacggaggaggaactccagaccgacctgggcatgaaatcgggcatcacccgcaagaggttctttagggagctcacggagctcaagaccttcgccaactattctacgtgcgaccgcagcaacctggcggactggctgggcagcctggacccgcgcttccgccagtacacctacggcctggtcagctgcggcctggaccgctccctgctgcaccgcgtgtctgagcagcagctgctggaagactgcggcatccacctgggcgtgcaccgcgcccgcatcctcacggcggccagagaaatgctacactccccgctgccctgtactggtggcaaacccagtggggacactccagatgtcttcatcagctaccgccggaactcaggttcccagctggccagtctcctgaaggtgcacctgcagctgcatggcttcagtgtcttcattgatgtggagaagctggaagcaggcaagttcgaggacaaactcatccagagtgtcatgggtgcccgcaactttgtgttggtgctatcacctggagcactggacaagtgcatgcaagaccatgactgcaaggattgggtgcataaggagattgtgactgctttaagctgcggcaagaacattgtgcccatcattgatggcttcgagtggcctgagccccaggtcctgcctgaggacatgcaggctgtgcttactttcaacggtatcaagtggtcccacgaataccaggaggccaccattgagaagatcatccgcttcctgcagggccgctcctcccgggactcatctgcaggctctgacaccagtttggagggtgctgcacccatgggtccaacctacccatacgatgttccagattacgcttaaggatcccgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctagacgcgtctggaacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattaattctgcagtcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaagaggggactggaagggctaattcactcccaacgaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggccttgacattgctagcgtttaccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatcaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt